SEQUENCE LISTING (14 sequences)

SEQ ID NO: 1 - DNA, 44 bp, Artificial Sequence
  Synthetic
cgatgaattcgccaccgctagcatggtgagcaagggcgaggagc

SEQ ID NO: 2 - DNA, 43 bp, Artificial Sequence
  Synthetic
ctcattactaaccggctagcttacttgtacagctcgtccatgc

SEQ ID NO: 3 - DNA, 55 bp, Artificial Sequence
  Synthetic
ccagcacagtggcggccgctcgagcatggtgagcaagggcgaggagctgttcacc

SEQ ID NO: 4 - DNA, 52 bp, Artificial Sequence
  Synthetic
ccgcgggccctctagactcgagttacttgtacagctcgtccatgccgagagt

SEQ ID NO: 5 - DNA, 39 bp, Artificial Sequence
  Synthetic
gtcatggatgaagaatccttgaacccagctttcttgtac

SEQ ID NO: 6 - DNA, 39 bp, Artificial Sequence
  Synthetic
gtacaagaaagctgggttcaaggattcttcatccatgac

SEQ ID NO: 7 - DNA, 1551 bp, Artificial Sequence
  Synthetic vector
gatatccagcacagtggcggccgctcgagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagcgacgaattttagtctactgaaacaagcgggagacgtggaggaaaaccctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagctcgagtctagagggcccgcggttaac

SEQ ID NO: 8 - DNA, 1556 bp, Artificial Sequence
  Synthetic vector
agtactctagaggatccactagtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggagcgacgaattttagtctactgaaacaagcgggagacgtggaggaaaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggcactagtccagtgtggtggaattctgcagatatc

SEQ ID NO: 9 - DNA, 33 bp, Artificial Sequence
  Synthetic
caagaaagctgggttcaacaggtgcagctcgtg

SEQ ID NO: 10 - DNA, 33 bp, Artificial Sequence
  Synthetic
cacgagctgcacctgttgaacccagctttcttg

SEQ ID NO: 11 - DNA, 37 bp, Artificial Sequence
  Synthetic
gtacaagaaagctgggttcaagacgggaaccattctg

SEQ ID NO: 12 - DNA, 37 bp, Artificial Sequence
  Synthetic
cagaatggttcccgtcttgaacccagctttcttgtac

SEQ ID NO: 13 - DNA, 37 bp, Artificial Sequence
  Synthetic
gaaagctgggttcaataaattttctcctggctggtac

SEQ ID NO: 14 - DNA, 37 bp, Artificial Sequence
  Synthetic
gtaccagccaggagaaaatttattgaacccagctttc

(19) United States
(12) Patent Application Publication
(10) Pub. No.: US 2022/0387602 A1
(43) Pub. Date: Dec. 8, 2022

(54) BIFUNCTIONAL DEGRADERS AND THEIR METHODS OF USE

(71) Applicant: NOVARTIS AG, Basel (CH)

(72) Inventors: Luca ARISTA, Riehen (CH); Valerie BROENNIMANN, Basel (CH); Pier Luca D'ALESSANDRO, Hegenheim (FR); Lionel DOUMAMPOUOM-METOUL, Rosenau (FR); Marie-Line GOUDE, BARTENHEIM (FR); Christina HEBACH, Muenchenstein (CH); Gregory John HOLLINGWORTH, Basel (CH); Ingrid Karen Jennifer JEULIN, VILLAGE-NEUF (FR); Louise Clare KIRMAN, Swampscott, MA (US); Julien LORBER, Colmar (FR); Fupeng MA, Melrose, MA (US); Anna VULPETTI, Basel (CH); Ken YAMADA, Chestnut Hill, MA (US); Thomas ZOLLER, Andolsheim (FR)

(21) Appl. No.: 17/642,285

(22) PCT Filed: Sep. 14, 2020
(86) PCT No.: PCT/IB2020/058535

§ 371 Date: Mar. 11, 2022

Related U.S. Application Data
(60) Provisional application No. 62/905,849, filed on Sep. 25, 2019.
(60) Provisional application No. 62/901,161, filed on Sep. 16, 2019.

(51) Int. Cl. A61K 47/55; A61K 47/54

(52) U.S. Cl. CPC A61K 47/55; A61K 47/545

(57) ABSTRACT

Described herein are bifunctional degrader compounds, their various targets, their preparation, pharmaceutical compositions comprising them, and their use in the treatment of conditions, diseases, and disorders mediated by various target proteins.

### CLAIM OF PRIORITY

This application claims the benefit of priority to U.S. Provisional Application No. 62/901,161 filed Sep. 16, 2019 and U.S. Provisional Application No. 62/905,849 filed Sep. 25, 2019, the disclosure of each of which is incorporated by reference herein in its entirety.

### FIELD OF THE DISCLOSURE

Described herein are bifunctional degrader compounds, their various targets, their preparation, pharmaceutical compositions comprising them, and their use in the treatment of conditions, diseases, and disorders mediated by various target proteins.

### REFERENCE TO A SEQUENCE LISTING

This application is filed with a Computer Readable Form of a Sequence Listing in accordance with 37 C.F.R. § 1.821(c). The text file submitted by EFS, “PAT058639-US-PSP_14293-889_sequence_listing.txt,” was created on Sep. 9, 2019, has a file size of 7 Kbytes, and is hereby incorporated by reference in its entirety.

### BACKGROUND

The Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases. These ligases comprise over 500 different proteins and are categorized into multiple classes defined by the structural element of their E3 functional activity.

Cereblon (CRBN) interacts with damaged DNA binding protein 1 and forms an E3 ubiquitin ligase complex with Cullin 4 where it functions as a substrate receptor in which the proteins recognized by CRBN might be ubiquitinated and degraded by proteasomes.

Proteasome-mediated degradation of unneeded or damaged proteins plays a very important role in maintaining regular cellular functions, such as cell survival, proliferation and growth. More recently, CRBN has been identified as the target of immunomodulatory drugs (IMiDs) like thalidomide and lenalinomide and is associated with teratogenicity and also the cytotoxicity of IMiDs which are widely used to treat multiple myeloma patients. Kronke et al., Science 343: 301-305 (2014); Petzold et al., Nature 532:127-130 (2016); Bjorklund et al., Blood Cancer J. 5, e354 (2015); Lu et al., Science 343:305-309 (2014); Gandhi et al., Br. J. Haematol. 164: 811-821 (2014).

The principle of induced degradation of protein targets as a potential therapeutic approach has been described by Crews, J. Med, Chem. 61(2): 403-404 (2018) and references cited therein. There is a need for selective target protein degraders and the present application addresses the generation of bifunctional degrader molecules that are directed to a variety of protein targets for in vivo target validation and as therapeutics.

### SUMMARY

In one aspect, the disclosure provides a bifunctional compound of Formula (I):

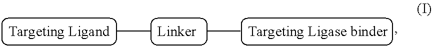

or a pharmaceutically acceptable salt, hydrate, solvate, prodrug, stereoisomer, or tautomer thereof, wherein:
the Targeting Ligand is a group that is capable of binding to a Target Protein; the Linker is a group that covalently links the Targeting Ligand to the Targeting Ligase Binder; and
the Targeting Ligase Binder is a group that is capable of binding to a ligase (e.g., Cereblon E3 Ubiquitin ligase).

In an embodiment, the Targeting Ligase Binder has a Formula (TLB-I):

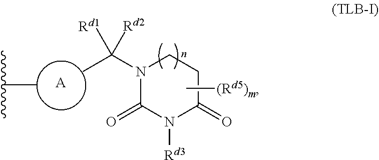

or a pharmaceutically acceptable salt, hydrate, solvate, prodrug, stereoisomer, or tautomer thereof, wherein:
[symbol] denotes the point of attachment to the Linker in Formula (I);
Ring A is a 6-membered aryl, or 5- or 6-membered heteroaryl, each of which is substituted with 0-4 occurrences of R^{d4}.
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d5} attached to the same carbon atom form a C₃₋₄ spirocycloalkyl;
R^{p} is H or C₁₋₆ alkyl;
m is 1 or 2; and
n is 1 or 2.

In an embodiment, n is 1. In an embodiment, R^{d3} is H. In an embodiment, R^{d3} is —CH₂OP(O)(OR^{p})₂.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a 5-membered heteroaryl. In an embodiment, A is a 5-membered nitrogen-containing heteroaryl. In an embodiment, A is a 6-membered heteroaryl. In an embodiment, ring A is a 6-membered nitrogen-containing heteroaryl. In an embodiment, ring A is pyridyl or pyridonyl. In an embodiment, R^{d4} is hydroxyl or C₁₋₆ alkoxyl.

In an embodiment, the Targeting Ligase Binder has a Formula (TLB-II):

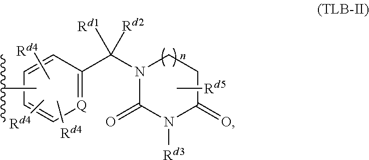

or a pharmaceutically acceptable salt, hydrate, solvate, prodrug, stereoisomer, or tautomer thereof, wherein:
[symbol] denotes the point of attachment to the Linker in Formula (I);
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, R^{d3} is H. In an embodiment, R^{d3} is —CH₂OP(O)(OR^{p})₂.

In an embodiment, R^{d4} is hydroxyl or C₁₋₆ alkoxyl.

In an embodiment, the Targeting Ligase Binder has a Formula (TLB-III):

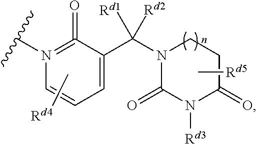

or a pharmaceutically acceptable salt, hydrate, solvate, prodrug, stereoisomer, or tautomer thereof, wherein:
[symbol] denotes the point of attachment to the Linker in Formula (I);
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, R^{d3} is H. In an embodiment, R^{d3} is —CH₂OP(O)(OR^{p})₂. In an embodiment, R^{d1} is H. In an embodiment, R^{d2} is H. In an embodiment, R^{d1} and R^{d2} are both H.

In an embodiment, the Targeting Ligase Binder has a Formula (TLB-IV):

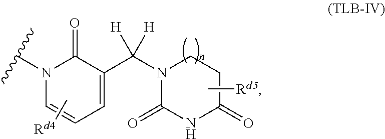

or a pharmaceutically acceptable salt, hydrate, solvate, prodrug, stereoisomer, or tautomer thereof, wherein:
[symbol] denotes the point of attachment to the Linker in Formula (I);
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl; and
n is 1 or 2.

In an embodiment, n is 1. In an embodiment, R^{d3} is H. In an embodiment, R^{d3} is —CH₂OP(O)(OR^{p})₂. In an embodiment, R^{d4} is H or C₁₋₃ alkyl. In an embodiment, R^{d4} is H. In an embodiment, R^{d5} is H or C₁₋₃ alkyl. In an embodiment, R^{d5} is H.

In an embodiment, the Targeting Ligase Binder has a Formula (TLB-V):

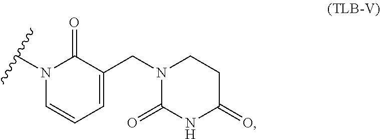 [SMILES: *n1cccc(CN2CCC(=O)NC2=O)c1=O]

or a pharmaceutically acceptable salt, hydrate, solvate, prodrug, stereoisomer, or tautomer thereof.

In an embodiment, the Targeting Ligase Binder has a Formula (TLB-VI):

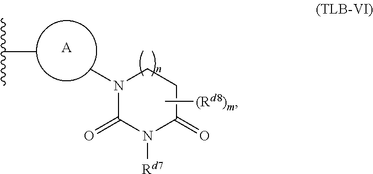

or a pharmaceutically acceptable salt, hydrate, solvate, prodrug, stereoisomer, or tautomer thereof, wherein:
[symbol] denotes the point of attachment to the Linker in Formula (I);
Ring A is a 6-membered aryl or 6-membered heteroaryl, each of which is independently substituted with 0-4 occurrences of R^{d6};
each R^{d6} is independently selected from the group consisting of H, hydroxyl, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{p} is H or C₁₋₆ alkyl;
each R^{d8} is independently selected from the group consisting of H, C₁₋₆ alkyl, halogen, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
m is 1 or 2; and
n is 1 or 2.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a nitrogen-containing 6-membered heteroaryl. In an embodiment, ring A is pyridyl.

In an embodiment, n is 1. In an embodiment, n is 2. In an embodiment, R^{d7} is —CH₂OP(O)(OR^{p})₂. In an embodiment, R^{d7} is H. In an embodiment, R^{d8} is H. In an embodiment, R^{d7} and R^{d8} are both H. In an embodiment, R^{d6} is H. In an embodiment, R^{d6} is selected from the group consisting of H, halogen, C₁₋₆ alkyl, and C₁₋₆ alkoxyl. In an embodiment, R^{d6} is selected from the group consisting of H, halogen, C₁₋₆ alkyl, and C₁₋₆ alkoxyl; and R^{d7}, and R^{d8} are each H.

In an embodiment, the Targeting Ligase Binder has a Formula (TLB-VII):

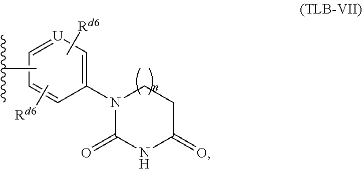

or a pharmaceutically acceptable salt, hydrate, solvate, prodrug, stereoisomer, or tautomer thereof, wherein:
[symbol] denotes the point of attachment to the Linker in Formula (I);
U is —CR^{d6} or N;
each R^{d6} is independently selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

In an embodiment, n is 1. In an embodiment, n is 2. In an embodiment, each R^{d6} is independently selected from the group consisting of H, halogen, C₁₋₃ alkyl, and C₁₋₃ alkoxy. In an embodiment, each R^{d6} is H. In an embodiment, one of R^{d6} is H. In an embodiment, one of R^{d6} is not H.

In an embodiment, the Targeting Ligase Binder has a Formula (TLB-VIII):

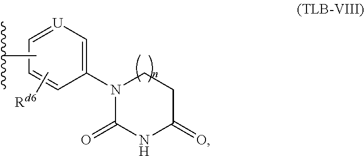

or a pharmaceutically acceptable salt, hydrate, solvate, prodrug, stereoisomer, or tautomer thereof, wherein:
[symbol] denotes the point of attachment to the Linker in Formula (I);
U is —CR^{d6} or N;
R^{d6} is selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

In an embodiment, the Targeting Ligase Binder has a Formula (TLB-IX):

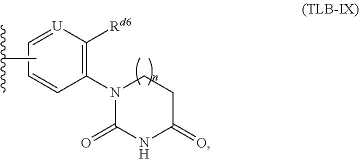

or a pharmaceutically acceptable salt, hydrate, solvate, prodrug, stereoisomer, or tautomer thereof, wherein:
[symbol] denotes the point of attachment to the Linker in Formula (I);
U is independently —CR^{d6} or N;
R^{d6} is selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

In an embodiment, n is 1. In an embodiment, n is 2. In an embodiment, U is N. In an embodiment, U is —CR^{d6}. In an embodiment, each R^{d6} is independently selected from the group consisting of H, methyl, halogen, methoxy, and methoxymethyl. In an embodiment, R^{d6} is H. In an embodiment, R^{d6} is methyl. In an embodiment, R^{d6} is halogen. In an embodiment, R^{d6} is methoxy.

In an embodiment, the Linker has Formula (L-I):

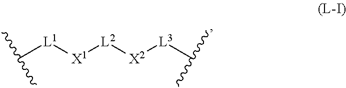

or a pharmaceutically acceptable salt, hydrate, solvate, prodrug, stereoisomer, or tautomer thereof, wherein:
L¹ is selected from the group consisting of a bond, O, NR′, C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand in Formula (I);
X¹ and X² are each independently selected from the group consisting of a bond, carbocyclyl, heterocyclyl, and heteroaryl;
L² is selected from the group consisting of a bond, O, NR′, C(O), C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from a bond, C₁₋₆ alkylene, C₂₋₆ alkenylene, C₂₋₆ alkynylene, C₁₋₆ heteroalkylene, C(O), S(O)₂, O, NR′, *C(O)—C₁₋₉ alkylene, *C(O)—C₁₋₆ alkylene-O, and *C(O)—C₁₋₉ heteroalkylene, wherein * denotes the point of attachment of L³ to X² in (L-I);
wherein no more than 2 of L¹, X¹, X², L², and L³ can simultaneously be a bond; and
R′ is hydrogen or C₁₋₆ alkyl.

In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl.

In an embodiment, one of X¹ and X² is a bond, and the other is a heterocyclyl. In an embodiment, X¹ and X² are each independently selected from piperidinyl and piperazinyl. In an embodiment, X¹ and X² are both piperidinyl. In an embodiment, —X¹-L²-X²— is:

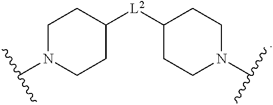

In an embodiment, the Linker is a compound having the following formula:

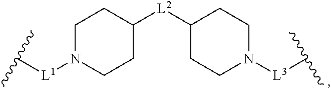

or a pharmaceutically acceptable salt, hydrate, solvate, prodrug, stereoisomer, or tautomer thereof.

In an embodiment, —X¹-L²-X²— forms a spiroheterocyclyl having the structure,

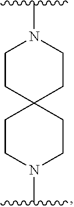 [SMILES: *N1CCC2(CC1)CCN(*)CC2]

substituted with 0-4 occurrences of R^{a}, wherein each R^{a} is independently selected from C₁₋₆ alkyl, C₁₋₆ alkoxyl, and C₁₋₆ hydroxyalkyl.

In an embodiment, —X¹-L²-X²— forms a spiroheterocyclyl having the structure,

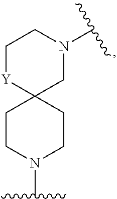

substituted with 0-4 occurrences of R^{b}, wherein Y is selected from CH₂, oxygen, and nitrogen; and each R^{b} is independently selected from C₁₋₆ alkyl, C₁₋₆ alkoxyl, and C₁₋₆ hydroxyalkyl.

In an embodiment, X¹ and X² are each a bond. In an embodiment, L³ is independently selected from the group consisting of —C(O)—, C₂₋₆ alkynylene, or C₁₋₆ heteroalkylene; and L¹ is —C(O)—, C₁₋₈ alkylene, C₁₋₈ heteroalkylene, and *C₁₋₆ alkylene-C(O). In an embodiment, L³ is selected from the group consisting of —C(O)—, —O—C₁₋₆ alkylene, C₂₋₆ alkynylene, and C₁₋₆ heteroalkylene; and L¹ is C₁₋₈ alkylene or C₁₋₈ heteroalkylene. In an embodiment, L³ is —C(O)— or C₁₋₆ heteroalkylene; and L¹ is C₁₋₈ alkylene or C₁₋₈ heteroalkylene. In an embodiment, L³ is a bond or —O—; and L¹ is —C(O)— or C₁₋₈ heteroalkylene. In an embodiment, L³ is selected from the group consisting of —O—, —C(O)—, —S(O)₂—, and C₁₋₆ heteroalkylene; and L¹ is C₁₋₈ alkylene or C₁₋₈ heteroalkylene. In an embodiment, L² is —C(O)—, —NR′—, or C₁₋₆ alkylene. In an embodiment, L² is —C(O)—, —O—, or C₁₋₆ alkylene. In an embodiment, L² is C₁₋₆ alkylene. In an embodiment, L² is selected from the group consisting of —C(O)—, C₁₋₆ alkylene, C₁₋₆ heteroalkylene, and *C(O)NR′—C₁₋₆ alkylene. In an embodiment, Y is CH₂, CH(C₁₋₃ alkyl), C(C₁₋₃ alkyl)₂, oxygen, NH, or N(C₁₋₃ alkyl).

In an embodiment, the Targeting Ligase Binder-Linker has Formula (TLB-L-I):

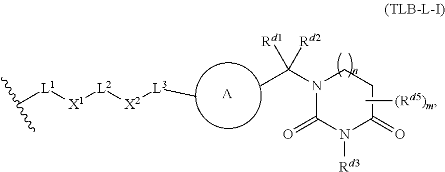

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′—, —C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of
L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O), —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-I);
wherein no more than 2 of L¹, X¹, X², L², and L³ can simultaneously be a bond;
Ring A is a 6-membered aryl, or 5- or 6-membered heteroaryl, each of which is substituted with 0-4 occurrences of R^{d4};
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d5} attached to the same carbon atom form a C₃₋₄ spirocycloalkyl;
R^{p} is H or C₁₋₆ alkyl;
m is 1 or 2; and
n is 1 or 2.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a 5-membered heteroaryl. In an embodiment, ring A is a 5-membered nitrogen-containing heteroaryl. In an embodiment, ring A is a 6-membered heteroaryl. In an embodiment, ring A is a 6-membered nitrogen-containing heteroaryl. In an embodiment, ring A is pyridyl. In an embodiment, n is 1. In an embodiment, R^{d3} is H. In an embodiment, R^{d3} is —CH₂OP(O)(OR^{p})₂.

In an embodiment, the Targeting Ligase Binder-Linker has Formula (TLB-L-II):

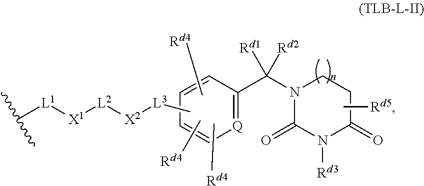

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′—, —C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O), —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-II);
wherein no more than 2 of L¹, X¹, X², L², and L³ can simultaneously be a bond;
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, R^{d3} is H. In an embodiment, R^{d3} is —CH₂OP(O)(OR^{p})₂.

In another embodiment, the Targeting Ligase Binder-Linker has Formula (TLB-L-III):

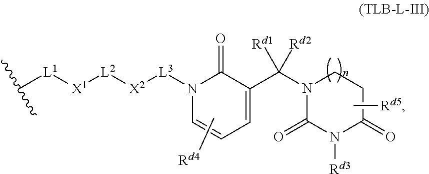

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and
C(O)NR′—C₁₋₆ alkylene, wherein * denotes the point of attachment of L² to X²; or X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-III);
wherein no more than 2 of L¹, X¹, X², L², and L³ can simultaneously be a bond;
R′ is hydrogen or C₁₋₆ alkyl;
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, R^{d3} is H. In an embodiment, R^{d3} is —CH₂OP(O)(OR^{p})₂.

In another embodiment, the Targeting Ligase Binder-Linker has Formula (TLB-L-IV):

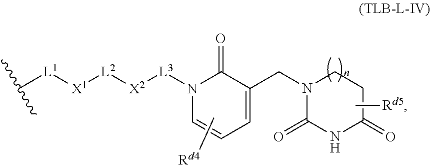

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of
L² to X²; or X¹-L²-X² form a spiroheterocyclyl; L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-IV);
wherein no more than 2 of L¹, X¹, X², L², and L³ can simultaneously be a bond;
R′ is hydrogen or C₁₋₆ alkyl;
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
n is 1 or 2.

In an embodiment, n is 1. In an embodiment, n is 2.

In another embodiment, the Targeting Ligase Binder-Linker has Formula (TLB-L-V):

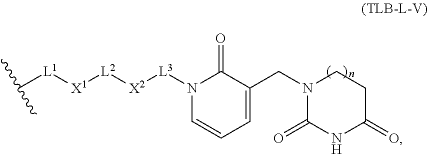

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-V);
wherein no more than 2 of L¹, X¹, X², L², and L³ can simultaneously be a bond;
R′ is hydrogen or C₁₋₆ alkyl; and
n is 1 or 2.

In an embodiment, n is 1. In an embodiment, n is 2. In an embodiment, L³ is selected from the group consisting of —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl. In an embodiment, one of X¹ and X² is a bond, and the other is a heterocyclyl.

In another embodiment, the Targeting Ligase Binder-Linker, or a pharmaceutically acceptable salt, hydrate, solvate, prodrug, stereoisomer, or tautomer thereof, has a Formula selected from:

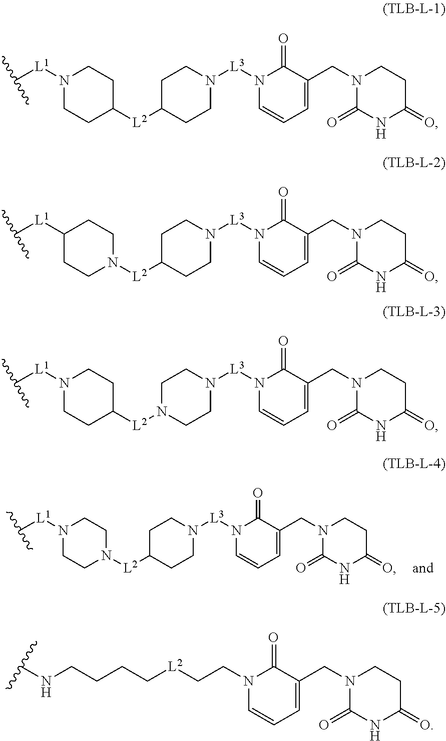

In another aspect, the compound has the Formula (BF-I):

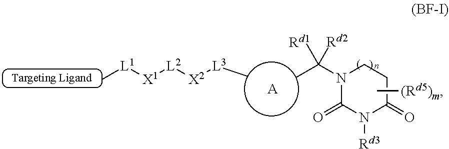

or a pharmaceutically acceptable salt, hydrate, solvate, prodrug, stereoisomer, or tautomer thereof, wherein:
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene;
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-I);
wherein no more than 2 of L¹, X¹, X², L², and L³ can simultaneously be a bond;
Ring A is a 6-membered aryl, or 5- or 6-membered heteroaryl, each of which is substituted with 0-4 occurrences of R^{d4}.
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d8} attached to the same carbon atom form a C₃₋₄ spirocycloalkyl;
R^{p} is H or C₁₋₆ alkyl;
m is 1 or 2; and
n is 1 or 2, wherein the Targeting Ligand is a group capable of binding to a Target Protein.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a 5-membered heteroaryl. In an embodiment, ring A is a 5-membered nitrogen-containing heteroaryl. In an embodiment, ring A is a 6-membered heteroaryl. In an embodiment, ring A is a 6-membered nitrogen-containing heteroaryl. In an embodiment, ring A is pyridyl.

In an embodiment, n is 1. In an embodiment, n is 2. In an embodiment, R^{d3} is —CH₂OP(O)(OR^{p})₂. In an embodiment, R^{d3} is H.

In another embodiment, the compound has the Formula (BF-II):

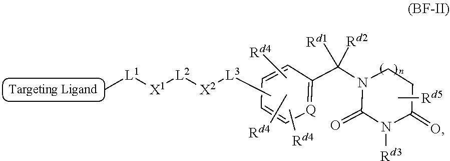

or a pharmaceutically acceptable salt, hydrate, solvate, prodrug, stereoisomer, or tautomer thereof, wherein:
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-II);
wherein no more than 2 of L¹, X¹, X², L², and L³ can simultaneously be a bond;
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2, wherein the Targeting Ligand is a group capable of binding to a Target Protein.

In an embodiment, n is 1. In an embodiment, n is 2. In an embodiment, R^{d3} is —CH₂OP(O)(OR^{p})₂. In an embodiment, R^{d3} is H.

In another embodiment, the compound has the Formula (BF-III):

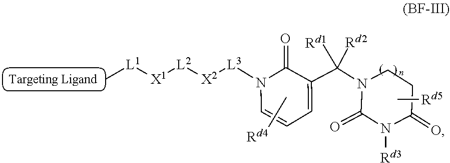

or a pharmaceutically acceptable salt, hydrate, solvate, prodrug, stereoisomer, or tautomer thereof, wherein:
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-III);
wherein no more than 2 of L¹, X¹, X², L², and L³ can simultaneously be a bond;
R′ is hydrogen or C₁₋₆ alkyl;
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2, wherein the Targeting Ligand is a group capable of binding to a Target Protein.

In an embodiment, n is 1. In an embodiment, n is 2. In an embodiment, R^{d3} is —CH₂OP(O)(OR^{p})₂. In an embodiment, R^{d3} is H. In an embodiment, —X¹-L²-X²— is:

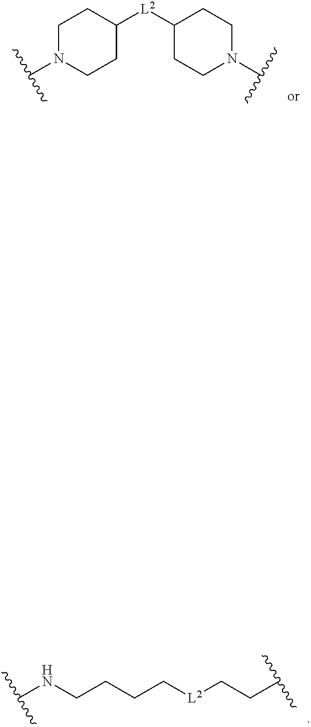

In an embodiment, L¹ is —O— or C₁₋₆ alkylene. In an embodiment, R^{d1} and R^{d2} are both methyl. In an embodiment, R^{d1} and R^{d2} are both H. In an embodiment, R^{d4} is H or C₁₋₃ alkyl. In an embodiment, R^{d5} is H or C₁₋₃ alkyl.

In another embodiment, the Targeting Ligase Binder-Linker has Formula (TLB-L-VI):

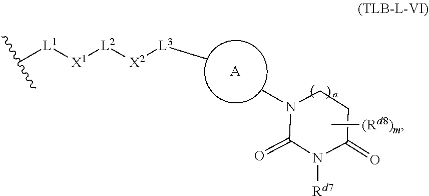

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-VI);
wherein no more than 2 of L¹, X¹, X², L², and L³ can simultaneously be a bond;
R′ is hydrogen or C₁₋₆ alkyl;
Ring A is a 6-membered aryl or 6-membered heteroaryl, each of which is independently substituted with 0-4 occurrences of R^{d6};
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{p} is H or C₁₋₆ alkyl;
each R^{d8} is independently selected from the group consisting of H, C₁₋₆ alkyl, halogen, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
m is 1 or 2; and
n is 1 or 2.

In an embodiment, n is 1. In an embodiment, n is 2. In an embodiment, R^{d3} is —CH₂OP(O)(OR^{p})₂. In an embodiment, R^{d3} is H.

In another embodiment, the Targeting Ligase Binder-Linker has Formula (TLB-L-VII):

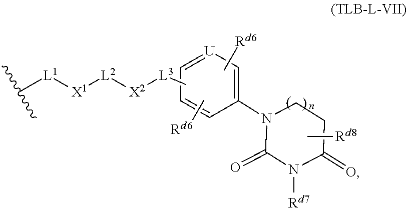

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-VII);
wherein no more than 2 of L¹, X¹, X², L², and L³ can simultaneously be a bond;
R′ is hydrogen or C₁₋₆ alkyl;
U is —CR^{d6} or N;
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d8} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, n is 2. In an embodiment, R^{d3} is —CH₂OP(O)(OR^{p})₂. In an embodiment, R^{d3} is H. In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl. In an embodiment, one of X¹ and X² is a bond, and the other is a heterocyclyl.

In an embodiment, the Targeting Ligase Binder-Linker has Formula (TLB-L-VIII or TLB-L-IX):

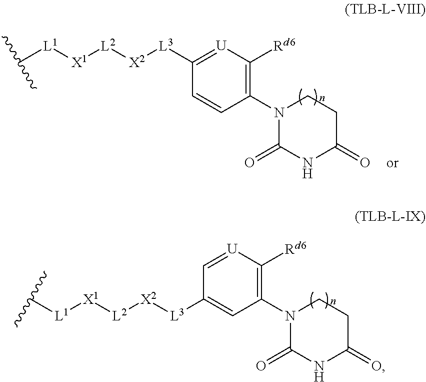

or a pharmaceutically acceptable salt, hydrate, solvate, prodrug, stereoisomer, or tautomer thereof, wherein the point of attachment to the Targeting Ligand is through L¹.

In an embodiment, n is 1. In an embodiment, n is 2.

In another embodiment, the Targeting Ligase Binder-Linker, or a pharmaceutically acceptable salt, hydrate, solvate, prodrug, stereoisomer, or tautomer thereof, has a Formula selected from:

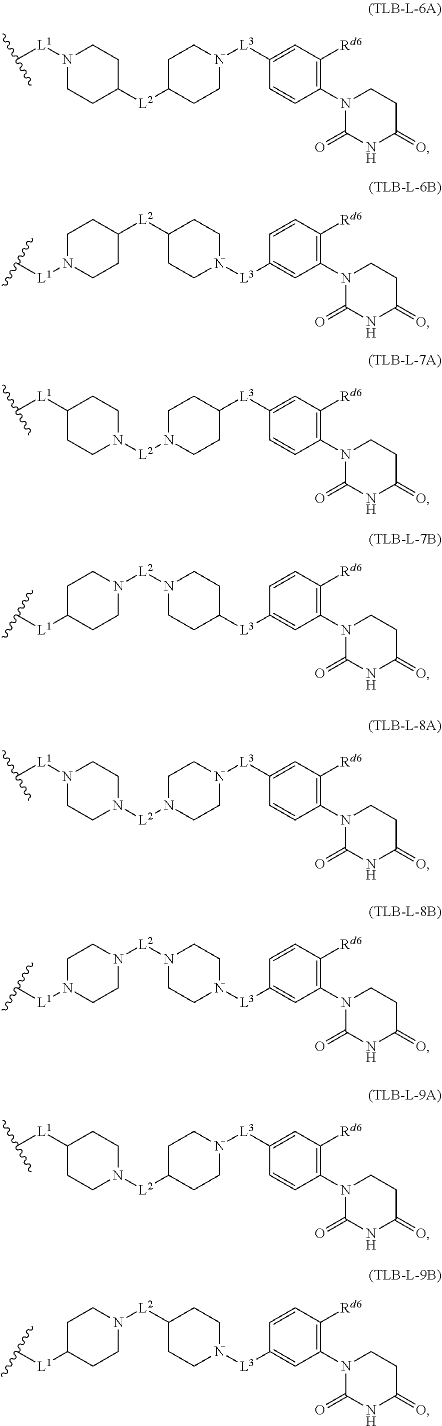

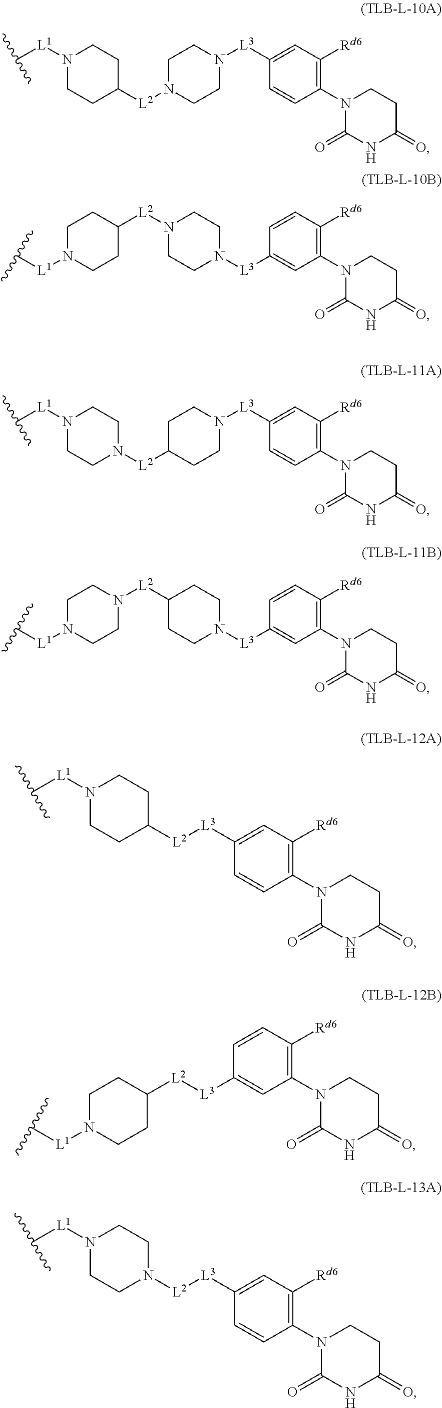

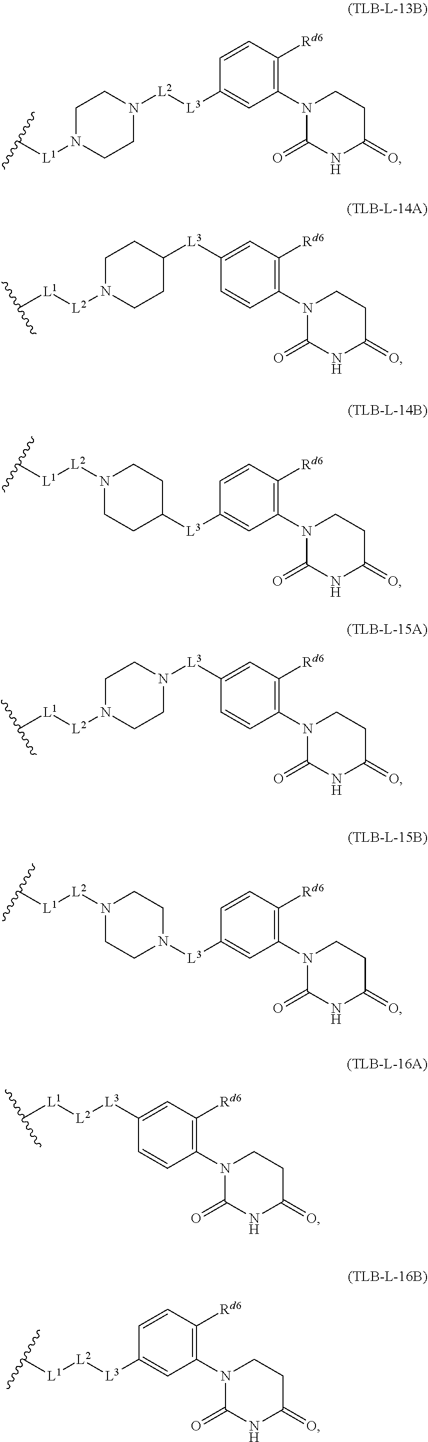

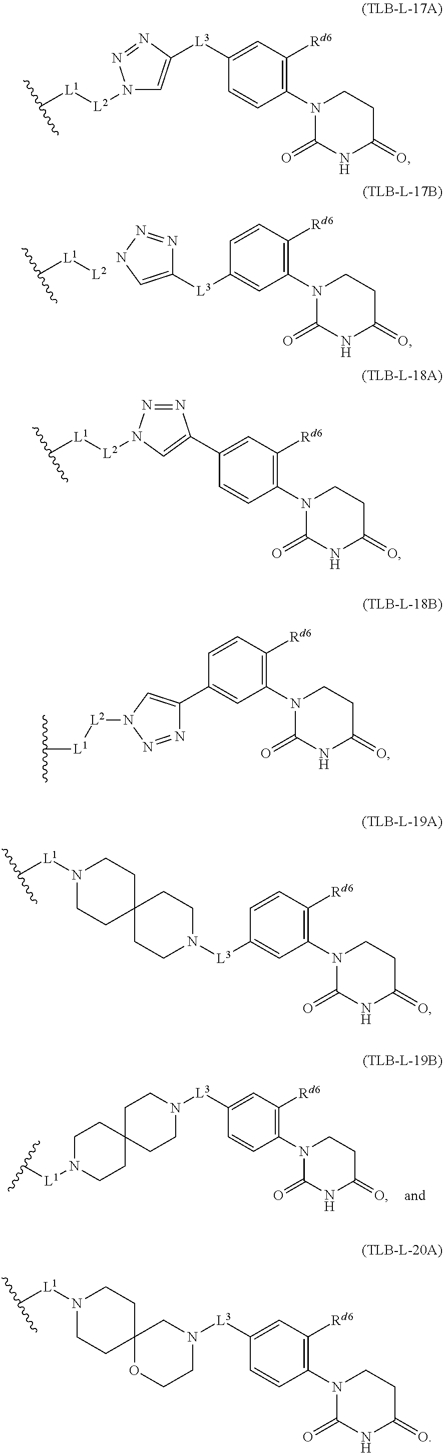

In another embodiment, the compound has the Formula (BF-IV):

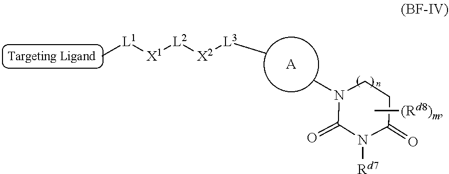

or a pharmaceutically acceptable salt, hydrate, solvate, prodrug, stereoisomer, or tautomer thereof, wherein:
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-IV);
wherein no more than 2 of L¹, X¹, X², L², and L³ can simultaneously be a bond;
R′ is hydrogen or C₁₋₆ alkyl;
Ring A is a 6-membered aryl or 6-membered heteroaryl, each of which is independently substituted with 0-4 occurrences of R^{d6};
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{p} is H or C₁₋₆ alkyl;
each R^{d8} is independently selected from the group consisting of H, C₁₋₆ alkyl, halogen, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
m is 1 or 2; and
n is 1 or 2, wherein the Targeting Ligand is a group capable of binding to a Target Protein.

In another embodiment, the compound has the Formula (BF-V-A or BF-V-B):

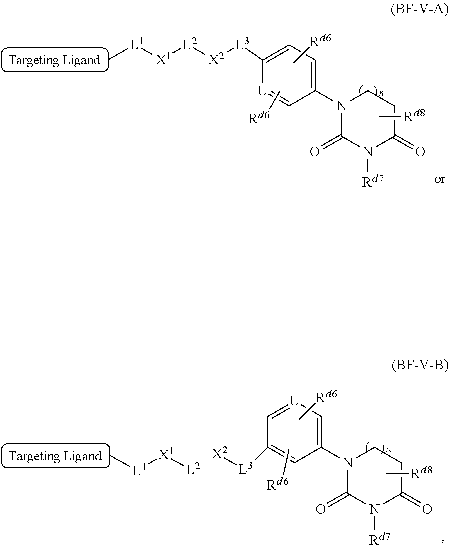

or a pharmaceutically acceptable salt, hydrate, solvate, prodrug, stereoisomer, or tautomer thereof, wherein:
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-V-A or BF-V-B);
wherein no more than 2 of L¹, X¹, X², L², and L³ can simultaneously be a bond;
R′ is hydrogen or C₁₋₆ alkyl;
U is —CR^{d6} or N;
each R^{d6} is independently selected from the group consisting of H, oxo, C₁₋₆ alkyl, halogen, C₁₋₆ alkoxyl, C₁₋₆ alkoxyalkyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d8} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2, wherein the Targeting Ligand is a group capable of binding to a Target Protein.

In an embodiment, n is 1. In an embodiment, n is 2. In an embodiment, R^{d7} is —CH₂OP(O)(OR^{p})₂. In an embodiment, R^{d7} is H. In an embodiment, U is —CR^{d6}. In an embodiment, R^{d8} is H. In an embodiment, R^{d7} and R^{d8} are each independently H. In an embodiment, R^{d6} is H. In an embodiment, R^{d6} is selected from the group consisting of H, halogen, C₁₋₆ alkyl, and C₁₋₆ alkoxyl. In an embodiment, R^{d6} is selected from the group consisting of H, halogen, C₁₋₆ alkyl, and C₁₋₆ alkoxyl; and R^{d7}, and R^{d8} are each H.

In another embodiment, L¹-X¹-L²-X²-L³ is selected from the group consisting of:

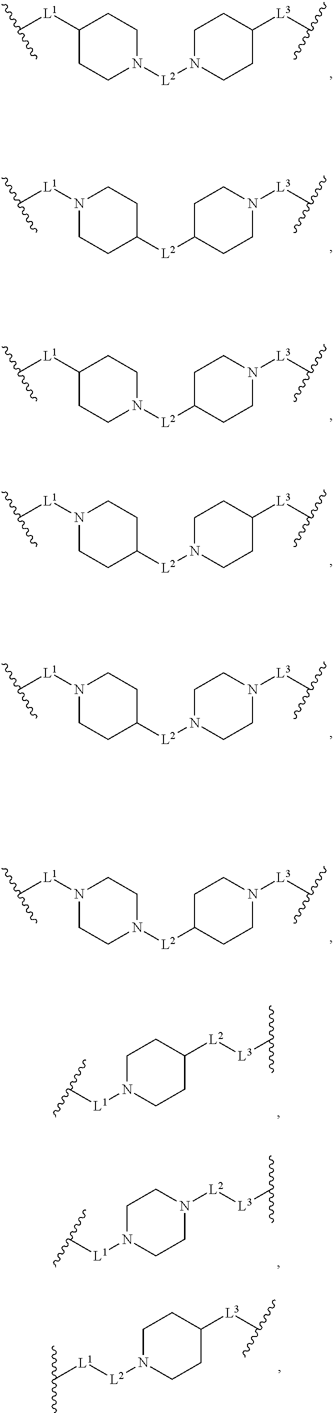

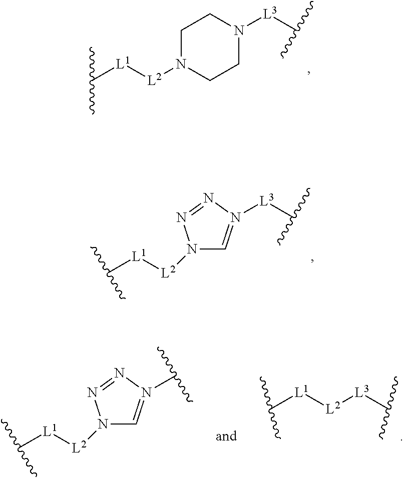

In an embodiment, L³ is selected from the group consisting of a bond, —O—, —C(O)—, —S(O)₂—, C₁₋₆ alkylene, C₂₋₆ alkynylene, and C₁₋₆ heteroalkylene.

In another embodiment, the Targeting is a BRD9 targeting ligand of Formula (BRD9-I):

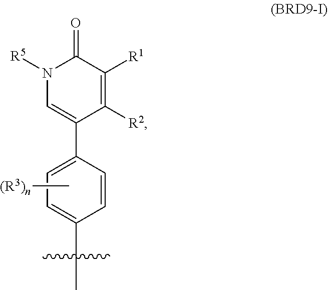

or a pharmaceutically acceptable salt, hydrate, solvate, prodrug, stereoisomer, or tautomer thereof, wherein:
R¹ and R² are independently selected from the group consisting of hydrogen and C₁₋₆ alkyl; or R¹ and R² together with the atoms to which they are attached form an aryl or heteroaryl;
R³ are each independently selected from the group consisting of C₁₋₆ alkyl, C₁₋₆ alkoxyl, and halogen;
R⁵ is selected from the group consisting of hydrogen and C₁₋₃ alkyl;
n is 0, 1, or 2.

In another embodiment, the Targeting Ligand is a BTK targeting ligand of Formula (BTK-I):

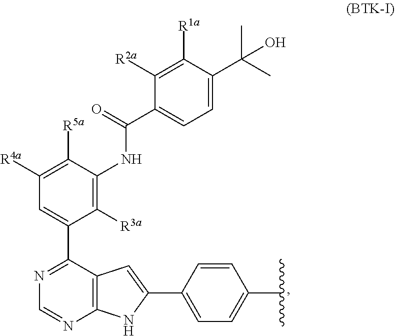

or a pharmaceutically acceptable salt, hydrate, solvate, prodrug, stereoisomer, or tautomer thereof, wherein:
R^{1a} is H or halo;
R^{2a} is halo;
R^{3a} is C₁₋₆ alkyl;
R^{4a} is halo; and
R^{5a} is H or halo.

Another embodiment is a pharmaceutical composition comprising a compound described herein, or a pharmaceutically acceptable salt, hydrate, solvate, prodrug, stereoisomer, or tautomer thereof, and a pharmaceutically acceptable carrier.

Another embodiment is a pharmaceutical combination comprising a compound described herein, or a pharmaceutically acceptable salt, hydrate, solvate, prodrug, stereoisomer, or tautomer thereof, and one or more additional therapeutic agent(s).

Another embodiment is a method for inducing degradation of a Target Protein in a subject in need thereof, the method comprising administering to the subject a therapeutically effective amount of a compound described herein, or a pharmaceutically acceptable salt, hydrate, solvate, prodrug, stereoisomer, or tautomer thereof.

Another embodiment is a method of inhibiting, reducing, or eliminating the activity of a Target Protein, the method comprising administering to the subject a compound described herein, or a pharmaceutically acceptable salt, hydrate, solvate, prodrug, stereoisomer, or tautomer thereof.

In an embodiment, inhibiting, reducing, or eliminating the activity of a Target Protein comprises recruiting a ligase (e.g., Cereblon E3 Ubiquitin ligase) with the Targeting Ligase Binder, e.g., a Targeting Ligase Binder described herein, of the bifunctional compound, e.g., a bifunctional compound described herein, forming a ternary complex of the Target Protein, bifunctional compound, and the ligase, to thereby inhibit, reduce or eliminate the activity of the Target Protein.

In an embodiment, the Target Protein is selected from Table 1:

TABLE 1
Exemplary Target Proteins

| Target Code | Target Name |
|---|---|
| a5b1 | Integrin a5b1 |
| AAK1 | Adaptor protein complex 2-associated protein kinase 1 |
| ABL1 | Abelson Tyrosine-Protein Kinase 1 including T314I |
| ACAT2 | Acetyl-CoA Acetyltransferase 2 |
| ADAR | adenosine deaminase RNA specific |
| ADORA2A | adenosine A2a receptor |
| AHR | Aryl hydrocarbon receptor |
| Akt | Protein Kinase B |
| ALK | ALK receptor tyrosine kinase |
| AR | Androgen hormone receptor including |
| ARAF | A-Raf proto-oncogene, serine/threonine kinase |
| ASC | Apoptosis-Associated Speck-Like |
| ATP4 | ATP Synthase Subunit 4 |
| AURKA | Aurora Kinase A |
| AURKB | Aurora Kinase B |
| BACH1 | BTB and CNC homology 1 |
| BACH2 | BTB and CNC homology 2 |
| BCL 2 | BCL2 apoptosis regulator |
| BCL11A | BAF Chromatin Remodeling Complex Subunit BCL11A |
| BCL2L1 | BCL2 like 1 |
| BCR-ABL | BCR-ABL protein |
| BKV JCV | Human Polyomaviruses - capsid |
| BLK | B-lymphoid tyrosine kinase |
| BRAF | B-Raf proto-oncogene, serine/threonine kinase |
| BRD2 | Bromodomain containing protein 2 |
| BRD3 | Bromodomain containing protein 3 |
| BRD4 | Bromodomain containing protein 4 |
| BRD5 | Bromodomain containing protein 5 |
| BRD7 | Bromodomain containing protein 7 |
| BRD8 | Bromodomain containing protein 8 |
| BRD9 | Bromodomain containing protein 9 |
| BRDT | bromodomain testis associated protein |
| BTK | Bruton's tyrosine kinase |
| BTK mutant | various Bruton's tyrosine kinase mutations |
| BTKC481S | mutant Bruton's tyrosine kinase |
| BTLA | B and T lymphocyte associated |
| CCR7 | C-C motif chemokine receptor 7 |
| CD274 | CD274 molecule |
| CD47 | CD47 molecule |
| CDK12 | Cyclin Dependent Kinase 12 |
| CDK13 | Cyclin Dependent Kinase 13 |
| CDK17 | Cyclin Dependent Kinase 17 |
| CDK2 | Cyclin Dependent Kinase 2 |
| CDK4 | Cyclin Dependent Kinase 4 |
| CDK5 | Cyclin Dependent Kinase 5 |
| CDK6 | Cyclin Dependent Kinase 6 |
| CDK7 | Cyclin Dependent Kinase 7 |
| CDK9 | Cyclin Dependent Kinase 9 |
| CDPK1 | Calcium Dependent Protein Kinase 1 |
| CDPK4 | Calcium Dependent Protein Kinase 4 |
| CDX2 | caudal type homeobox 2 |
| CEBPA | CCAAT enhancer binding protein alpha |
| cGAS | Cyclic GMP-AMP Synthase ATI |
| c-Met | Met tyrosine-protein kinase or hepatocyte growth factor receptor |
| | (HGFR) |
| CRABP-I/II | Cellular retinoic acid-binding protein 1/2 |
| CREBBP | CREB binding protein |
| CSF1 | colony stimulating factor 1 |
| CSK | C-terminal Src kinase |
| CTLA4 | cytotoxic T-lymphocyte associated protein 4 |
| CTNNB1 | catenin beta 1 |
| DAPK1 | Death-associated protein kinase 1 |
| DDX58 | DExD/H-Box Helicase 58 |
| DHFR | Dihydrofolate reductase |
| DHODH | Dihydroorotate dehydrogenase |
| DNMT3A | DNA methyltransferase 3 alpha, WT and variant R882H, C, or P |
| DOT1L | DOT1L like histone lysine methyltransferase |
| DXR | 1-deoxy-D-xylulose 5-phosphate reductoisomerase |
| EBF1 | EBF transcription factor 1 |
| EGFR | Epidermal growth factor receptor |
| EGFR L858R | Epidermal growth factor receptor L859→R mutation |
| EGFR Δ Exon 19 | Epidermal growth factor receptor exon 19 deletion |
| ENTPD2 | ectonucleoside triphosphate diphosphohydrolase 2 |
| EP300 | E1A binding protein p300 |
| EPAS1 | Endothelial PAS domain protein 1 |
| ER | Estrogen receptor |
| ER | Estrogen receptor mutant including ESR Y573S/C/N; ESR D538G |
| ERRα | Estrogen-related receptor alpha |
| ERα | Estrogen receptor alpha |
| ESR1 | estrogen receptor 1 |
| Fak | Focal adhesion kinase |
| FARS2 | alpha subunit of the cytosolic phenylalanyl-tRNA synthetase (Pf PheRS) |
| FER | FER Tyrosine Kinase |
| FES | FES Tyrosine Kinase |
| FKBP12 | Peptidyl-prolyl cis-trans isomerase FKBP12 |
| FLI1 | Fli-1 proto-oncogene, ETS transcription factor |
| FLT3 | Receptor-type tyrosine-protein kinase FLT3 |
| FOXA1 | forkhead box A1 |
| GAK | Cyclin G-associated kinase |
| GATA3 | GATA binding protein 3 |
| GATA6 | GATA binding protein 6 |
| GSK3 | Serine/threonine protein kinase 3 |
| HAVCR2 | hepatitis A virus cellular receptor 2 |
| HBV | Hepatitis B virus - capsid |
| HCMV | cytomegalovirus - capsid |
| HCV | Hepatitis C virus- - capsid |
| HDAC1 | Histone Deacetylase 1 |
| HDAC10 | Histone Deacetylase 10 |
| HDAC11 | Histone Deacetylase 11 |
| HDAC2 | Histone Deacetylase 2 |
| HDAC3 | Histone Deacetylase 3 |
| HDAC4 | Histone Deacetylase 4 |
| HDAC5 | Histone Deacetylase 5 |
| HDAC6 | Histone Deacetylase 6 |
| HDAC7 | Histone Deacetylase 7 |
| HDAC8 | Histone Deacetylase 8 |
| HDAC9 | Histone Deacetylase 9 |
| HER2 | Human epidermal growth factor receptor 2 |
| HHV 6A and 6B | Human herpesvirus 6A and 6B - capsid |
| HHV7 | Human herpesvirus 7 - capsid |
| HHV-8 | Kaposi's sarcoma-associated herpesvirus - capsid |
| HIF1A | hypoxia inducible factor 1 subunit alpha |
| HIV | Human immunodeficiency virus -- capsid |
| HNF1A | HNF1 homeobox A |
| HNF1B | HNF1 homeobox B |
| HNF4A | hepatocyte nuclear factor 4 alpha |
| HRV | Rhinovirus and enteroviruses, including polioviruses, Coxsackie A |
| | viruses (CA), Coxsackie B viruses (CB), and echoviruses |
| HSD17b13 | hydroxysteroid 17-beta dehydrogenase 13 |
| HSV1 | Herpes simplex viruses 1 - capsid |
| HSV1 | Herpes simplex viruses 2 - capsid |
| HTT | Huntingtin |
| ICOS | Inducible T cell costimulator |
| IDH1 | Isocitrate dehydrogenase (NADP(+)) 1, including R132H/S/G/L/I |
| IDH2 | Isocitrate dehydrogenase (NADP(+)) 2, including R132H/S/G/L/I |
| IKZF1 | Ikaros transcription factors |
| IKZF3 | Aiolos transcription factors |
| IL15 | interleukin 15 |
| IL27 | interleukin 27 |
| IRAK1 | Interleukin-1 receptor-associated kinase 1 |
| IRAK1/4 | Interleukin-1 receptor-associated kinase 4 |
| IRF4 | interferon regulatory factor 4 |
| ITK | Interleukin-2-inducible T-cell kinase |
| JAK1 | Janus kinase 1 |
| JAK2 | Janus kinase 2 |
| JAK2 | Janus kinase 2, WT abd variant V617F |
| KARS1 | Lysyl-tRNA-Synthetase |
| KLF5 | Kruppel like factor 5 |
| KLRK1 | killer cell lectin like receptor K1 |
| KRAS | K-Ras P21 Protein |
| KRAS | KRAS proto-oncogene, GTPase, WT and variants G12V, C, D or G13D |
| KRAS G12V/C/D | mutant K-Ras P21 Protein |
| KRAS G13D | mutant K-Ras P21 Protein |
| LAG3 | lymphocyte activating 3 |
| LATS1 | Large Tumor Suppressor Kinase 1 |
| LATS2 | Large Tumor Suppressor Kinase 2 |
| LCAT | Lecithin-Cholesterol Acyltransferase |
| LCK | Protein tyrosine kinase Lck |
| LIMK2 | Leucine-rich repeat kinase 2 |
| LMO2 | LIM domain only 2 |
| M1 and M17 | M1 and M17 aminopeptidases |
| aminopeptidases | |
| MAP2K1 | mitogen-activated protein kinase kinase 1 |
| MAP2K2 | mitogen-activated protein kinase kinase 2 |
| MAPK1 | mitogen-activated protein kinase 1 |
| MAPK3 | mitogen-activated protein kinase 3 |
| MARK2 | Microtubule Affinity Regulating Kinase 2 |
| MAX | MYC associated factor X |
| MCL1 | MCL1 apoptosis regulator, BCL2 family member |
| MetAP-2 | Methionyl Aminopeptidase 2 |
| MICA | MHC class I polypeptide-related sequence A |
| MICB | MHC class I polypeptide-related sequence B |
| MITF | melanocyte inducing transcription factor |
| MLKL | Mixed lineage kinase domain-like protein |
| MLL | KMT2A (MLL) lysine methyltransferase 2A |
| MPL | MPL proto-oncogene, thrombopoietin receptor, WT and variant |
| | W515S, L or A |
| MRK | MO15-related protein kinase |
| MUC1 | mucin 1, cell surface associated |
| MYB | MYB proto-oncogene, transcription factor |
| MYBL2 | MYB proto-oncogene like 2 |
| MYC | MYC proto-oncogene, bHLH transcription factor |
| MYCN | MYCN proto-oncogene, bHLH transcription factor |
| MyD88 | Myeloid Differentiation Primary Response 88 |
| MYOC | Myocilin |
| NFE2L2 | nuclear factor, erythroid 2 like 2 |
| NPM1 | nucleophosmin 1, WT and variant NPM1c (AML frameshift mut) |
| Nr2E3 | Photoreceptor-Specific Nuclear Receptor |
| NRAS | NRAS proto-oncogene, GTPase, WT and variants Q61R, K, or L |
| Nrl | Neural Retina Leucine Zipper |
| NT5E | 5′-nucleotidase ecto |
| NTRK | SLIT and NTRK 3 [neurotrophic receptor tyrosine kinase 3] |
| p16INK4a | CDKN2A (P16INK4A) cyclin dependent kinase inhibitor 2A |
| p21CIP/WAF1 | Zinc finger protein Gfi-1? |
| p38 | Mitogen-activated protein (MAP) kinase (MAPK11) |
| PARS2 | cytoplasmic prolyl-tRNA (transfer RNA) synthetase (PfcPRS) |
| PAX8 | paired box 8 |
| PDCD1 | programmed cell death 1 |
| PDE4 | Phosphodiesterase 4 |
| PI3K34 | Phosphatidylinositol 3-kinase 34 |
| PIK3CA | phosphatidylinositol-4,5-bisphosphate 3-kinase catalytic subunit alpha |
| | WT and variants H1047R or E545K |
| PIK3CB | Phosphatidylinositol-4,5-bisphosphate 3-kinase catalytic subunit beta |
| PIK3CD | Phosphatidylinositol-4,5-bisphosphate 3-kinase catalytic subunit delta |
| PKA | cAMP dependent protein kinase |
| PKG | Protein kinase G |
| Plasmepsin IX | Plasmepsin IX |
| Plasmepsin X | Plasmepsin X |
| PNPLA2 | Patatin like phospholipase domain containing 2 |
| PNPLA3 | Patatin like phospholipase domain containing 2 |
| POU2AF1 | POU class 2 homeobox associating factor 1 |
| PPP1R12B | Protein phosphatase 1 regulatory subunit 12B |
| PRKAA1 | Protein Kinase AMP-Activated Catalytic Subunit Alpha 1 |
| PRMT5 | Protein arginine methyltransferase 5 |
| PRS | Prolyl-tRNA-Synthetase |
| PSD-95 | Post synaptic density protein 95, a membrane associated guanylate kinase |
| PTK2 | Protein Tyrosine Kinase 2 |
| PTK2B | Protein Tyrosine Kinase 2 beta |
| PTPN11 | protein tyrosine phosphatase non-receptor type 11 |
| PTPN2 | protein tyrosine phosphatase non-receptor type 2 |
| PTPN6 | protein tyrosine phosphatase non-receptor type 6 |
| PTPRB | Protein Tyrosine Phosphatase Receptor Type B |
| RAC1 | Rac family small GTPase 1, WT and variant P29S |
| RAET1E | retinoic acid early transcript 1E |
| RAET1G | retinoic acid early transcript 1G |
| RAET1L | retinoic acid early transcript 1L |
| RAF1 | Raf-1 proto-oncogene, serine/threonine kinase |
| RAR | Retinoic acid receptor |
| RET | ret proto-oncogene |
| RHOA | ras homolog family member A |
| RHOB | ras homolog family member B |
| RIPK2 | Receptor Interacting Serine/Threonine Kinase 2 |
| ROCK 1 | Rho Associated Coiled-Coil Containing Protein Kinase 1 |
| RSV | Human respiratory syncytial virus - capsid |
| RUNX1 | RUNX family transcription factor 1 |
| SARM1 | Sterile Alpha And TIR Motif-Containing Protein 1 |
| SHOC2 | SHOC2 leucine rich repeat scaffold protein |
| Sirt 1 | Sirtuin 1 |
| Sirt 2 | Sirtuin 2, an NAD-dependent deacetylase |
| Sirt 3 | Sirtuin 3 |
| Smad3 | SMAD Family Member 3 |
| SMARCA2 | SWI/SNF related, matrix associated, actin dependent regulator of |
| | chromatin, subfamily a, member 2 |
| SMARCA4 | SWI/SNF related, matrix associated, actin dependent regulator of |
| | chromatin, subfamily a, member 4 |
| SNAIL1 | Snail Family Transcriptional Repressor 1 |
| SNCA | Alpha synuclein |
| SOX10 | SRY-box transcription factor 10 |
| SPI1 | Spi-1 proto-oncogene |
| SPTLC1 | Serine palmitoyltransferase 1 |
| STAT1 | Signal transducer and activator of transcription 1 |
| STAT2 | Signal transducer and activator of transcription 2 |
| STAT3 | Signal transducer and activator of transcription 3 |
| STAT4 | Signal transducer and activator of transcription 4 |
| STAT5 | Signal transducer and activator of transcription 5 |
| STAT6 | Signal transducer and activator of transcription 6 |
| STK17A | Death-Associated Protein Kinase-Related 1 |
| STK17B | Death-Associated Protein Kinase-Related 2 |
| TACC3 | Transforming Acidic Coiled-Coil Containing Protein 3 |
| TANK | TRAF Family Member Associated NFKB Activator |
| Tau | Tau protein |
| TBK1 | TANK binding kinase 1 |
| TCF7L2 | transcription factor 7 like 2 |
| TDP43 | TAR DNA-binding protein 43 |
| TEAD1 | TEA domain transcription factor 1 |
| TEAD2 | TEA domain transcription factor 2 |
| TEAD3 | TEA domain transcription factor 3 |
| TEAD4 | TEA domain transcription factor 4 |
| TEC | Tec non-receptor protein-tyrosine kinase |
| TFAP2A | Transcription factor AP-2 alpha |
| TFAP2C | Transcription factor AP-2 gamma |
| TGFB1 | Transforming growth factor beta 1 |
| TIGIT | T cell immunoreceptor with Ig and ITIM domains |
| TLR7 | Toll like receptor 7 |
| TMEM173/STING1 | Stimulator of interferon response cGAMP interactor 1 |
| TNFRSF18 | TNF receptor superfamily member 18 |
| TNNI3K | TNNI3 Interacting Kinase |
| TP63 | tumor protein p63 |
| TRIM24 | Tripartite Motif Containing 24; transcriptional intermediary factor 1α |
| TYK2 | Tyrosine Kinase 2 |
| ULBP1 | UL16 binding protein 1 |
| ULBP2 | UL16 binding protein 2 |
| ULBP3 | UL16 binding protein 3 |
| ULK1 | Unc-51 Like Autophagy Activating Kinase 1 |
| VHL | Von Hippel-Lindau Tumor Suppressor |
| VZV | Varicella-zoster virus - capsid |
| WEE1 | WEE1 G2 Checkpoint Kinase |
| WRN | WRN RecQ like helicase |
| WWTR1 | WW domain containing transcription regulator 1 |
| XIAP | X-linked inhibitor of apoptosis |
| X-Protein | Hepatitis B X-protein. |
| ZFP64 | ZFP64 zinc finger protein. |

In an embodiment, the Target Protein is a fusion target protein. In an embodiment, the fusion target protein is selected from Table 2:

TABLE 2
Exemplary Fusion Target Proteins

| NCBI | NCBI | | |
|---|---|---|---|
| symbol | gene ID | Gene Name (extended from NCBI) | Example fusion partners (NCBI symbol) |
| RUNX1 | 861 | RUNX family transcription factor 1 | ETV6, MECOM, RUNX1T1 |
| ABL1 | 25 | ABL proto-oncogene 1 | BCR, NUP214, EML1, ETV6 |
| TCF3 | 6929 | transcription factor 3 | PBX1, TFPT, ZNF384, HLF |
| ZNF384 | 171017 | zinc finger protein 384 | EWSR1, TAF15, TCF3, EP300, |
| | | | CREBBP |
| CRLF2 | 64109 | cytokine receptor like factor 2 | P2RY8 |
| MEF2D | 4209 | myocyte enhancer factor 2D | BCL9, SS18, FOXJ2, CSF1R, DAZAP1 |
| PAX5 | 5079 | paired box 5 | ELN, ETV6, AUTS2, POM121, JAK2, |
| | | | FOXP1, NCOR1 |
| TRBC1 | 28639 | T cell receptor beta constant 1 | TAL1, TAL2, LYL1, OLIG2, LMO1, |
| | | | LMO2, TLX1, TLX3, MYC, and MYB |
| TRAC | 28755 | T cell receptor alpha constant | TAL1, TAL2, LYL1, OLIG2, LMO1, |
| | | | LMO2, TLX1, TLX3, MYC, and MYB |
| KMT2A | 4297 | lysine methyltransferase 2A | AFF1, MLLT3, MLLT1, MLLT10, |
| | | | MLLT4, ELL, AFDN, MLLT6, AFF4 |
| FLI1 | 2313 | Fli-1 proto-oncogene | EWSR1 |
| ERG | 2078 | ETS transcription factor ERG | TMPRSS2, EWSR1, SLC45A3, FUS, |
| | | | ETV6 |
| ETV1 | 2115 | ETS variant transcription factor 1 | TMPRSS2, EWSR1, SLC45A3, |
| | | | ACSL3, FOXP1 |
| ETV4 | 2118 | ETS variant transcription factor 4 | TMPRSS2, EWSR1, KLK2, CANT1, |
| | | | DDX5 |
| ETV5 | 2119 | ETS variant transcription factor 5 | TMPRSS2, SLC45A3 |
| PML | 5371 | promyelocytic leukemia | RARA |
| NUP98 | 4928 | nucleoporin 98 | NSD1, HOXA9, PRRX1, HOXA11, |
| | | | HOXA13, HOXC11, HOXC13 |
| CBFA2T3 | 863 | CBFA2/RUNX1 partner | GLIS2 |
| | | transcriptional co-repressor 3 | |
| CBFB | 865 | core-binding factor, beta subunit | MYH11 |
| KAT6A | 7994 | lysine acetyltransferase 6A | CREBBP, EP300, NCOA2 |
| FGFR3 | 2261 | fibroblast growth factor receptor 3 | TACC3, BAIAP2L1 |
| FGFR2 | 2263 | fibroblast growth factor receptor 2 | BICC1, KLK2, CIT, CCAR2 |
| FGFR1 | 2260 | fibroblast growth factor receptor 1 | FN1, TACC1 |
| RET | 5979 | ret proto-oncogene | CCDC6, ERC1, NCOA4, KIF5B, |
| | | | TRIM33, CUX1, CLIP1, RUFY3 |
| ALK | 238 | anaplastic lymphoma receptor | EML4, KIF5B, RANBP2, TFG, |
| | | tyrosine kinase | PRKAR1A, TPM3, NPM1 |
| NTRK3 | 4916 | neurotrophic tyrosine kinase, | ETV6, BTBD1, AML4, TFG, LYN, |
| | | receptor, type 3 | RBPMS |
| NTRK2 | 4915 | neurotrophic receptor tyrosine | QKI, NACC2, VCL, AGBL4, PAN3, |
| | | kinase 2 | AFAP1, DAB2IP |
| NTRK1 | 4914 | neurotrophic receptor tyrosine | CD74, MPRIP, TPM3, TFG, TPR |
| | | kinase 1 | |
| ROS1 | 6098 | ROS proto-oncogene 1, receptor | CD74, EZR, TPM3, SDC4, LRIG3, |
| | | tyrosine kinase | FIG, CCDC6, GOPC |
| RSPO3 | 84870 | R-spondin 3 | PTPRK |
| RSPO2 | 340419 | R-spondin 2 | EIFE3 |
| SMYD3 | 64754 | SET and MYND domain containing | KIF26B |
| | | 3 | |
| MAML3 | 55534 | mastermind like transcriptional | UBTF, PAX3, BCOR, TCF4 |
| | | coactivator 3 | |
| MAML2 | 84441 | mastermind like transcriptional | CRTC1 |
| | | coactivator 2 | |
| TFE3 | 7030 | transcription factor binding to | SFPQ, PRCC, ASPSCR1, NONO, |
| | | IGHM enhancer 3 | CLTC |
| TFEB | 7942 | transcription factor EB | MALAT1 |
| EGFR | 1956 | epidermal growth factor receptor | SEPT14 |
| BRAF | 673 | B-Raf proto-oncogene, | KIAA1549, PAPSS1, TRIM24, |
| | | serine/threonine kinase | TAX1BP1, CDC27, SND1 |
| SS18 | 6760 | SS18 subunit of BAF chromatin | SSX1, SSX2, SSX4 |
| | | remodeling complex | |
| PAX3 | 5077 | paired box 3 | FOXO1, MLLT7, NCOA1 |
| PAX7 | 5081 | paired box 7 | FOXO1 |
| EWSR1 | 2130 | EWS RNA binding protein 1 | DDIT3, CREB3L1, CREB3L2, CREB1, |
| | | | PBX1, ATF1, NR4A3 |
| FUS | 2521 | FUS RNA binding protein | ERG, CREB3L2, DDIT3 |
| CIC | 23152 | capicua transcriptional repressor | DUX4, DUX4L10, FOXO4 |
| FOSB | 2354 | FosB proto-oncogene, AP-1 | ZFP36, SERPINE1 |
| | | transcription factor | |
| VGLL2 | 245806 | vestigial like family member 2 | CITED2, NCOA2 |
| VAV1 | 7409 | vav guanine nucleotide exchange | GSS, MYO1F |
| | | factor 1 | |
| STAT6 | 6778 | signal transducer and activator of | NAB2 |
| | | transcription 6 | |
| HMGA2 | 8091 | high mobility group AT-hook 2 | LPP |
| PDGFRB | 5159 | platelet derived growth factor | ETV6 |
| | | receptor beta | |
| BRD4 | 23476 | bromodomain containing 4 | NUTM1 |
| PAX8 | 7849 | paired box 8 | PPARG |

Another embodiment is a method of treating a Target Protein-mediated disorder, disease, or condition in a patient comprising administering to the patient any of the compounds described herein, or a pharmaceutically acceptable salt, hydrate, solvate, prodrug, stereoisomer, or tautomer thereof. In an embodiment, the disorder is selected from a respiratory disorder, a proliferative disorder, an autoimmune disorder, an autoinflammatory disorder, an inflammatory disorder, a neurological disorder, and an infectious disease or disorder. In an embodiment, the disorder is a proliferative disorder. In an embodiment, the proliferative disorder is cancer.

Another embodiment is a method of treating cancer in a subject in need thereof, the method comprising administering to the subject a therapeutically effective amount of a compound of any one of the preceding claims, or a pharmaceutically acceptable salt thereof.

Another embodiment is a compound of Formula (ILB-I):

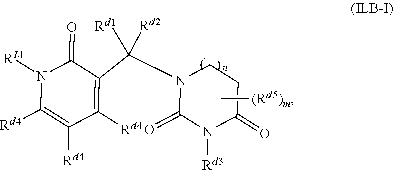

or a pharmaceutically acceptable salt, hydrate, solvate, prodrug, stereoisomer, or tautomer thereof, wherein:
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, C₁₋₆ alkyl, halogen, C₁₋₆ haloalkyl, C₁₋₆ alkoxyl; C₁₋₆ alkoxyalkyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d5} attached to the same carbon atom form a C₃₋₄ spirocycloalkyl;
R^{L1} is selected from the group consisting of C₁₋₆ alkyl, C₂₋₆ alkenyl, C₂₋₆ alkynyl, C₂₋₆ hydroxyalkyl, —(CH₂)₂₋₆NHR^{c}, C₃₋₆ heteroalkyl, C₂₋₆ haloalkyl, —(CH₂)₁₋₃C(O)OH, —(CH₂)₁₋₃C(O)H, —(CH₂)₁₋₃O(CH₂)₁₋₃C(O)H, —(CH₂)₀₋₃C₃₋₇ carbocyclyl, —(CH₂)₀₋₃ heterocyclyl, C₆ aryl, and heteroaryl, wherein the carbocyclyl, heterocyclyl, aryl, and heteroaryl is substituted with 0-2 occurrences of —O-heterocyclyl, —O-carbocyclyl, —C(O)— heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, or C₁₋₆ heteroalkyl;
R^{p} is H or C₁₋₆ alkyl;
m is 1 or 2; and
n is 1 or 2.

In an embodiment, n is 1. In an embodiment, n is 2. In an embodiment, R^{d3} is —CH₂OP(O)(OR^{p})₂. In an embodiment, R^{d3} is H.

In an embodiment, the compound or a pharmaceutically acceptable salt, hydrate, solvate, prodrug, stereoisomer, or tautomer thereof, is selected from:

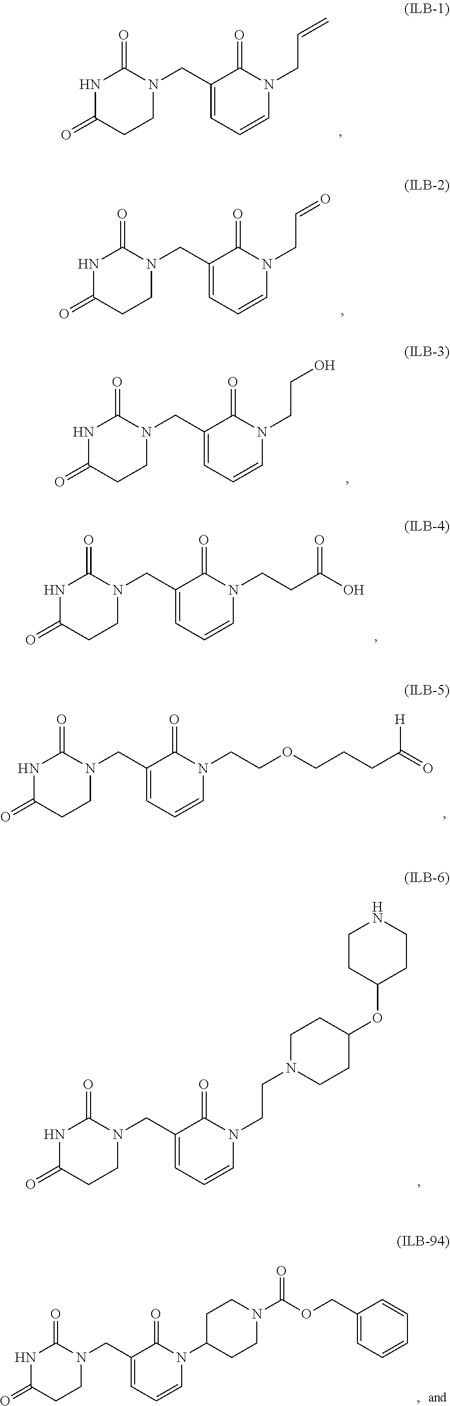

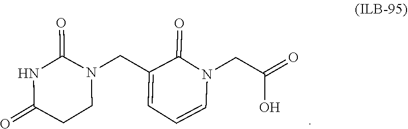 [SMILES: O=C(O)Cn1cccc(CN2CCC(=O)NC2=O)c1=O]

Another embodiments is a compound of Formula (ILB-II):

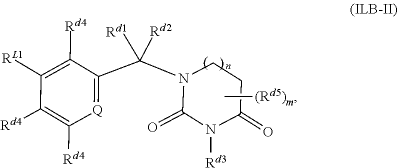

or a pharmaceutically acceptable salt, hydrate, solvate, prodrug, stereoisomer, or tautomer thereof, wherein:
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂(O)(CH₂)₂Si(CH₃)₃, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, C₁₋₆ alkyl, halogen, C₁₋₆ haloalkyl, C₁₋₆ alkoxyl; C₁₋₆ alkoxyalkyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d5} attached to the same carbon atom form a C₃₋₄ spirocycloalkyl;
R^{L1} is selected from the group consisting of C₂₋₆ alkenyl, C₂₋₆ alkynyl, C₂₋₆ hydroxyalkyl, —(CH₂)₂₋₆NHR^{c}, C₃₋₆ heteroalkyl, C₂₋₆ haloalkyl, —(CH₂)₀₋₃C(O)OH, —(CH₂)₀₋₃C(O)H, —(CH₂)₀₋₃C₃₋₇ carbocyclyl, —(CH₂)₀₋₃ heterocyclyl, C₆ aryl, and heteroaryl, wherein the carbocyclyl, heterocyclyl, aryl, and heteroaryl is substituted with 0-2 occurrences of —O-heterocyclyl, —O-carbocyclyl, —C(O)-heterocyclyl, —C(O)— carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, or C₁₋₆ heteroalkyl;
R^{p} is H or C₁₋₆ alkyl;
m is 1 or 2; and
n is 1 or 2.

In an embodiment, n is 1. In an embodiment, n is 2. In an embodiment, R^{d3} is —CH₂OP(O)(OR^{p})₂. In an embodiment, R^{d3} is H.

Another embodiment is a compound or a pharmaceutically acceptable salt, hydrate, solvate, prodrug, stereoisomer, or tautomer thereof, selected from:

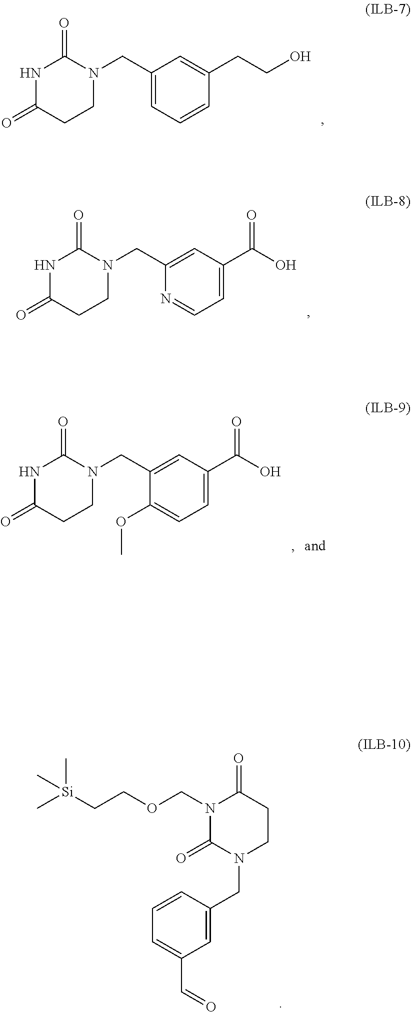 [SMILES: O=C1CCN(Cc2cccc(CCO)c2)C(=O)N1 ; O=C1CCN(Cc2cc(C(=O)O)ccn2)C(=O)N1 ; COc1ccc(C(=O)O)cc1CN1CCC(=O)NC1=O ; C[Si](C)(C)CCOCN1C(=O)CCN(Cc2cccc(C=O)c2)C1=O]

Another embodiment is a compound of Formula (ILB-III):

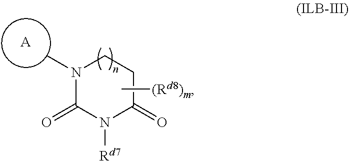

or a pharmaceutically acceptable salt, hydrate, solvate, prodrug, stereoisomer, or tautomer thereof, wherein:
Ring A is selected from the group consisting of:

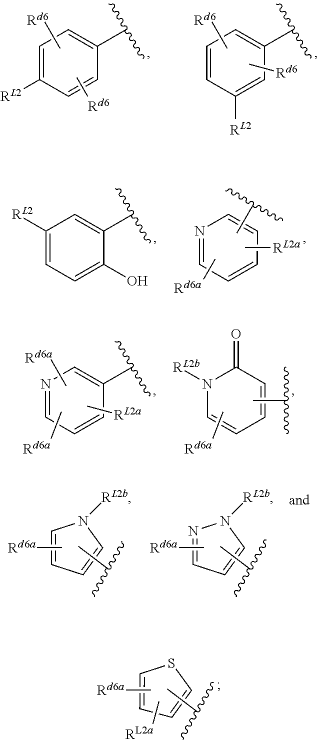

[symbol] denotes the point of attachment to the base molecule of (ILB-III);
each R^{d6} is independently selected from the group consisting of H, oxo, polyethylene glycol (PEG), halogen, C₁₋₃ alkyl, C₁₋₃ alkoxyl, C₁₋₆ haloalkyl, C₁₋₆ heteroalkyl, and —OC₁₋₇ heteroalkyl;
each R^{d6a} is independently selected from the group consisting of H, hydroxyl, oxo, polyethylene glycol (PEG), halogen, C₁₋₃ alkyl, C₁₋₃ alkoxyl, C₁₋₆ haloalkyl, C₁₋₆ heteroalkyl, and —OC₁₋₇ heteroalkyl;
R^{d7} is H, —CH₂OC(O)R^{p}, —CH₂OP(O)OHOR^{p}, —CH₂OP(O)(R^{p})₂, and —CH₂OP(O)(OR^{p})₂;
each R^{d8} is independently selected from the group consisting of H, C₁₋₆ alkyl, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
R^{L2} is selected from the group consisting of, hydroxyl, C₂₋₆ alkenyl, C₂₋₆ alkynyl, C₂₋₆ hydroxyalkyl, —(CH₂)₂₋₆NHR^{c}, —(CH₂)₂₋₆NR^{c}R^{d}, —O—(CH₂)₂₋₆NHR^{c}, C₄₋₈ heteroalkyl, C₂₋₆ haloalkyl, —SO₂—NH—(CH₂)₂₋₆NHR^{c}, —(CH₂)₀₋₃C(O)OH, —(CH₂)₀₋₃C(O)OR^{c}, —O—C₂₋₆ alkenyl, —O—(CH₂)₀₋₃C(O)H, —(CH₂)₀₋₃C(O)H, —O—(CH₂)₁₋₃C(O)OH, —(CH₂)₀₋₃ heterocyclyl, —C(O)—(CH₂)₀₋₃ heterocyclyl, —O—(CH₂)₀₋₃ heterocyclyl, —O—(CH₂)₀₋₃C(O)-heterocyclyl, —C₂₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, —(CH₂)₂₋₆N(R^{c})₂, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl wherein the heterocyclyl may optionally be substituted with halogen;
R^{L2a} is selected from the group consisting of H, hydroxyl, C₂₋₆ alkenyl, C₂₋₆ alkynyl, C₂₋₆ hydroxyalkyl, —(CH₂)₂₋₆NHR^{c}, —(CH₂)₂₋₆NR^{c}R^{d}, —O—(CH₂)₂₋₆NHR^{c}, C₁₋₈ heteroalkyl, C₁₋₆ haloalkyl, —SO₂—NH—(CH₂)₂₋₆NHR^{c}, —(CH₂)₀₋₃C(O)OH, —(CH₂)₀₋₃C(O)OR^{c}, —O—C₂₋₆ alkenyl, —O—(CH₂)₀₋₃C(O)H, —(CH₂)₀₋₃C(O)H, —O—(CH₂)₁₋₃C(O)OH, —(CH₂)₀₋₃ heterocyclyl, —C(O)—(CH₂)₀₋₃ heterocyclyl, —O—(CH₂)₀₋₃ heterocyclyl, —O—(CH₂)₀₋₃C(O)-heterocyclyl, —C₂₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, —(CH₂)₂₋₆N(R^{c})₂, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl, wherein the heterocyclyl may optionally be substituted with halogen;
R^{L2b} is selected from the group consisting of H, polyethylene glycol (PEG), C₁₋₃ alkyl, C₃₋₆ cycloalkyl, C₃₋₆ alkenyl, C₃₋₆ alkynyl, C₂₋₆ hydroxyalkyl, —(CH₂)₂₋₆NHR^{c}, —(CH₂)₂₋₆NR^{c}R^{d}, C₂₋₆ heteroalkyl, C₂₋₆ haloalkyl, —(CH₂)₁₋₃C(O)OH, —(CH₂)₁₋₃C(O)H, —(CH₂)₀₋₃ heterocyclyl, —C(O)—(CH₂)₀₋₃ heterocyclyl, —C₃₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, C₁₋₆ heteroalkyl, and —C(O)OC₁₋₆ alkyl;
R^{d} is H or C₁₋₄ alkyl; or R^{c} and R^{d} together with the nitrogen atom to which they are attached form a heterocyclyl substituted with 0-2 occurrences of —O-heterocyclyl,
R^{p} is H or C₁₋₆ alkyl;
m is 1 or 2; and
n is 1 or 2.

In an embodiment, ring A is selected from the group consisting of:

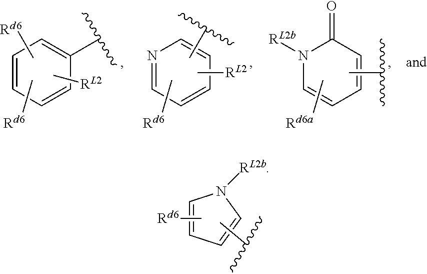

In an embodiment, n is 1. In an embodiment, n is 2. In an embodiment, R^{d7} is —CH₂OP(O)(OR^{p})₂. In an embodiment, R^{d7} is H.

Another embodiment is a compound or a pharmaceutically acceptable salt, hydrate, solvate, prodrug, stereoisomer, or tautomer thereof, selected from:

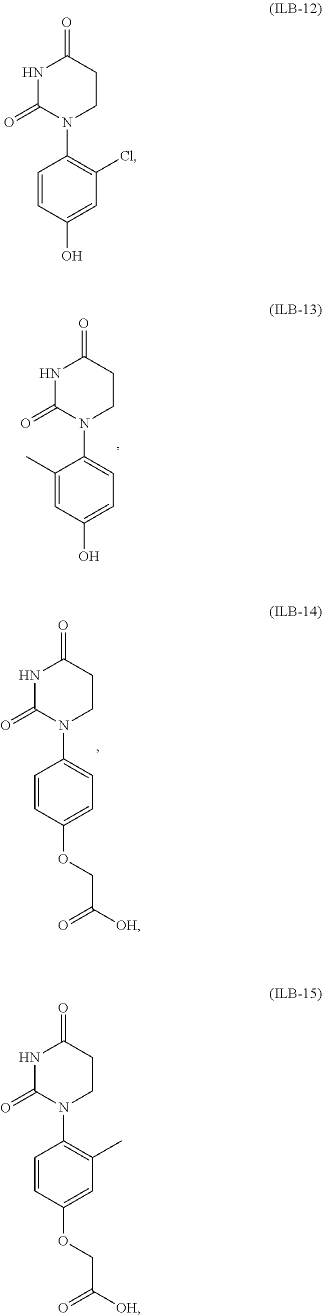 [SMILES: O=C1CCN(c2ccc(O)cc2Cl)C(=O)N1 ; Cc1cc(O)ccc1N1CCC(=O)NC1=O ; O=C(O)COc1ccc(N2CCC(=O)NC2=O)cc1 ; Cc1cc(OCC(=O)O)ccc1N1CCC(=O)NC1=O]

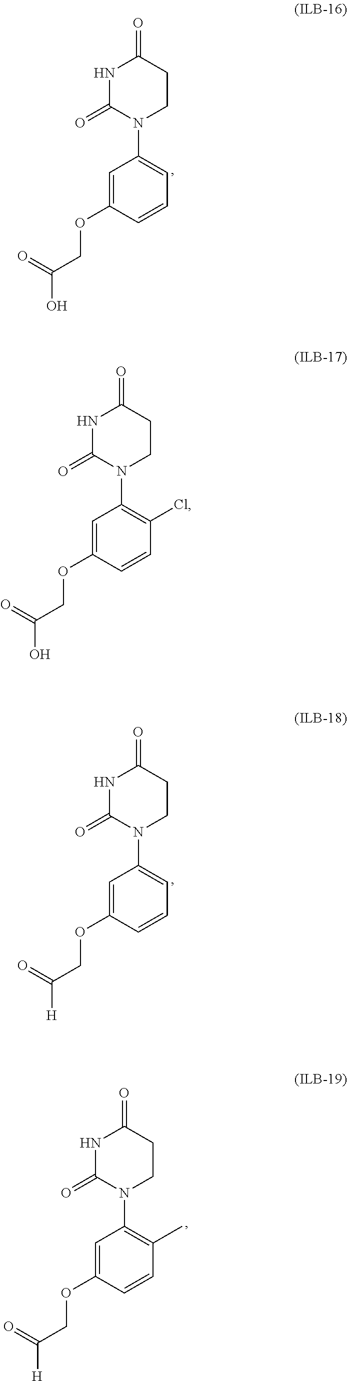 [SMILES: O=C(O)COc1cccc(N2CCC(=O)NC2=O)c1 ; O=C(O)COc1ccc(Cl)c(N2CCC(=O)NC2=O)c1 ; O=CCOc1cccc(N2CCC(=O)NC2=O)c1 ; Cc1ccc(OCC=O)cc1N1CCC(=O)NC1=O]

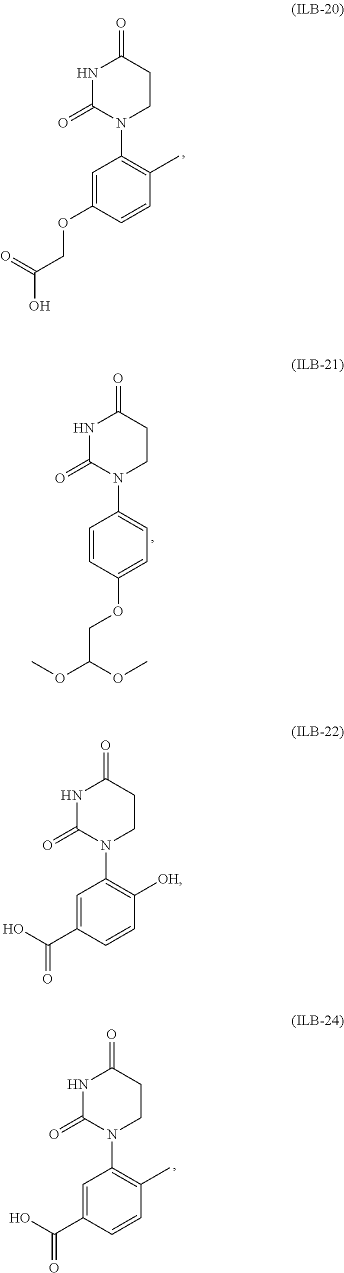

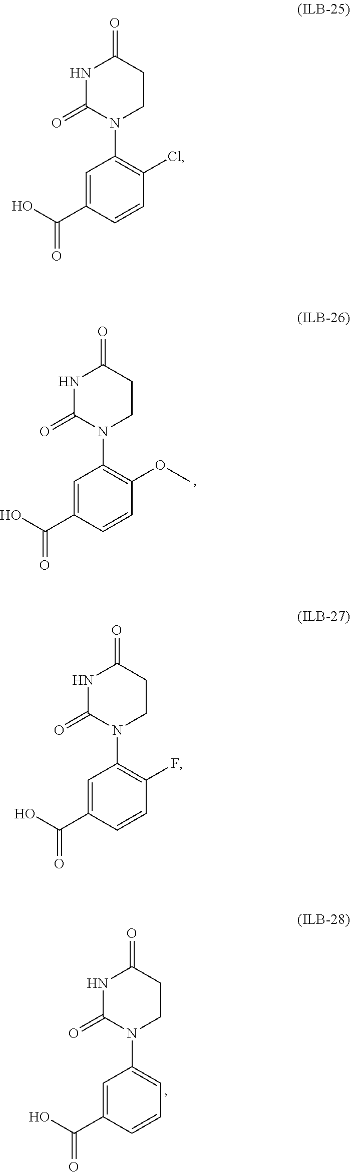 [SMILES: O=C1CCN(c2cc(C(=O)O)ccc2Cl)C(=O)N1 ; COc1ccc(C(=O)O)cc1N1CCC(=O)NC1=O ; O=C1CCN(c2cc(C(=O)O)ccc2F)C(=O)N1 ; O=C1CCN(c2cccc(C(=O)O)c2)C(=O)N1]

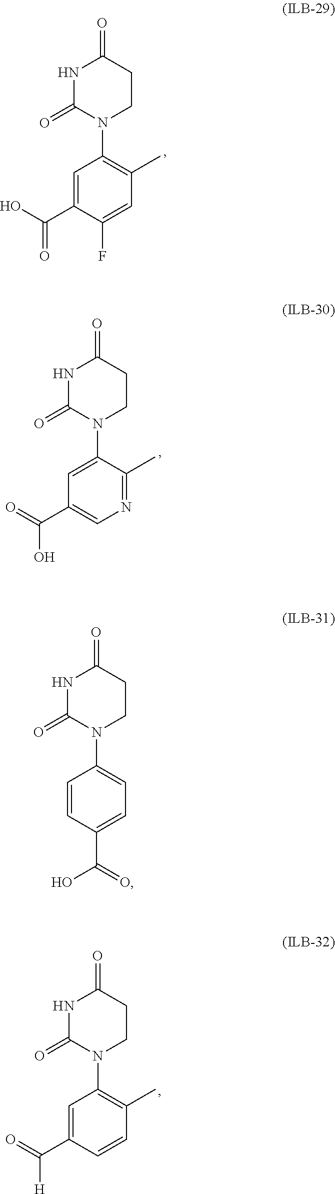 [SMILES: Cc1cc(F)c(C(=O)O)cc1N1CCC(=O)NC1=O ; Cc1ncc(C(=O)O)cc1N1CCC(=O)NC1=O ; O=C1CCN(c2ccc(C(=O)O)cc2)C(=O)N1 ; Cc1ccc(C=O)cc1N1CCC(=O)NC1=O]

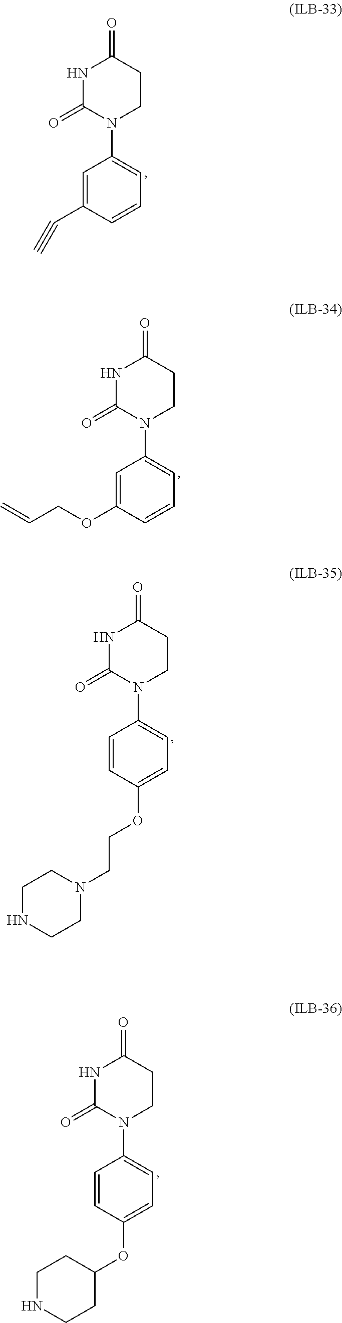 [SMILES: C#Cc1cccc(N2CCC(=O)NC2=O)c1 ; C=CCOc1cccc(N2CCC(=O)NC2=O)c1 ; O=C1CCN(c2ccc(OCCN3CCNCC3)cc2)C(=O)N1 ; O=C1CCN(c2ccc(OC3CCNCC3)cc2)C(=O)N1]

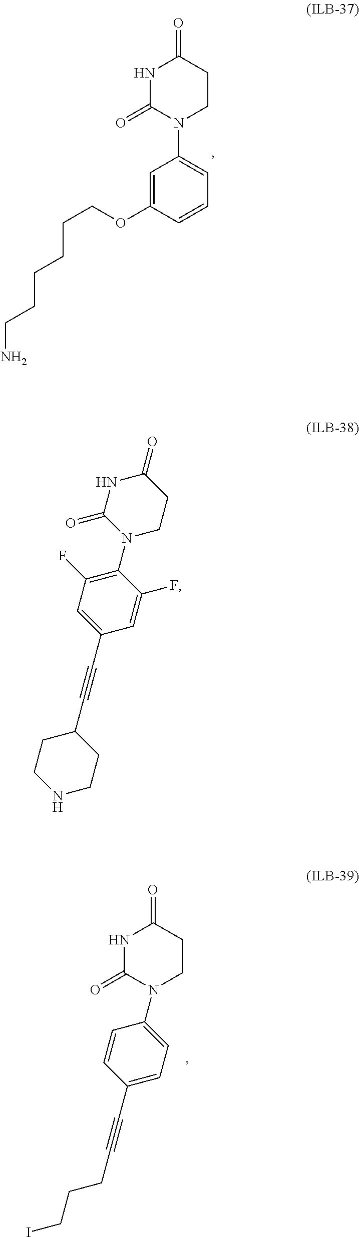 [SMILES: NCCCCCCOc1cccc(N2CCC(=O)NC2=O)c1 ; O=C1CCN(c2c(F)cc(C#CC3CCNCC3)cc2F)C(=O)N1 ; O=C1CCN(c2ccc(C#CCCCI)cc2)C(=O)N1]

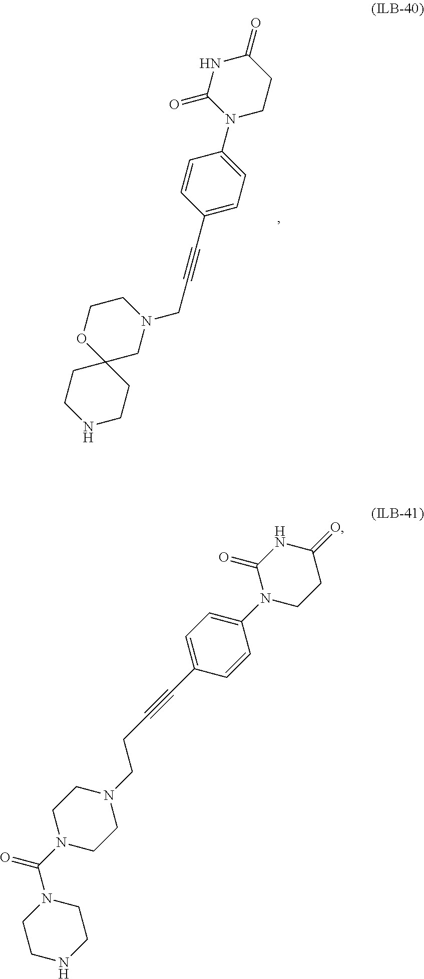 [SMILES: O=C1CCN(c2ccc(C#CCN3CCOC4(CCNCC4)C3)cc2)C(=O)N1 ; O=C1CCN(c2ccc(C#CCCN3CCN(C(=O)N4CCNCC4)CC3)cc2)C(=O)N1]

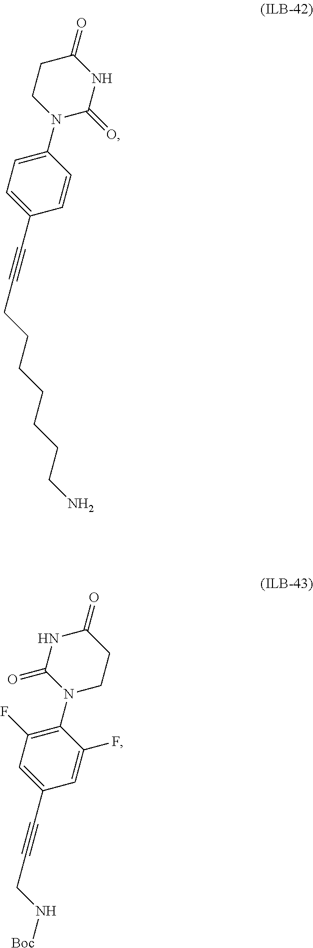 [SMILES: NCCCCCCCC#Cc1ccc(N2CCC(=O)NC2=O)cc1 ; CC(C)(C)OC(=O)NCC#Cc1cc(F)c(N2CCC(=O)NC2=O)c(F)c1]

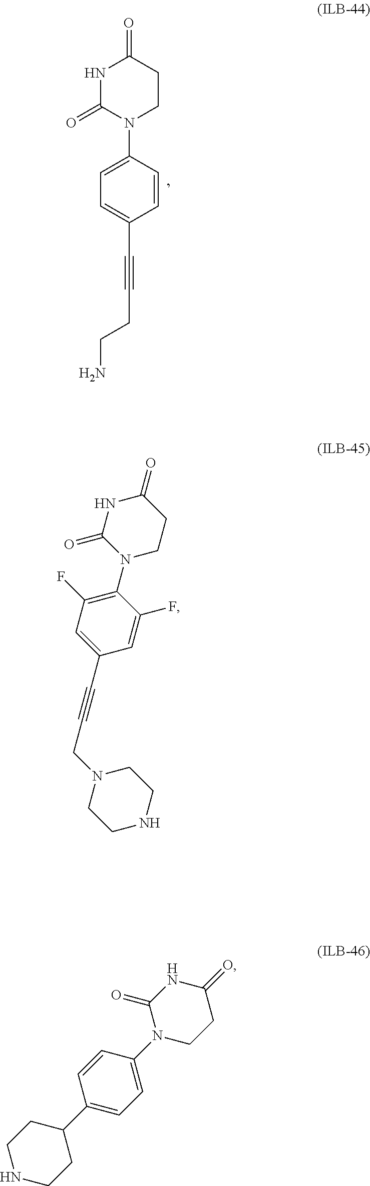 [SMILES: NCCC#Cc1ccc(N2CCC(=O)NC2=O)cc1 ; O=C1CCN(c2c(F)cc(C#CCN3CCNCC3)cc2F)C(=O)N1 ; O=C1CCN(c2ccc(C3CCNCC3)cc2)C(=O)N1]

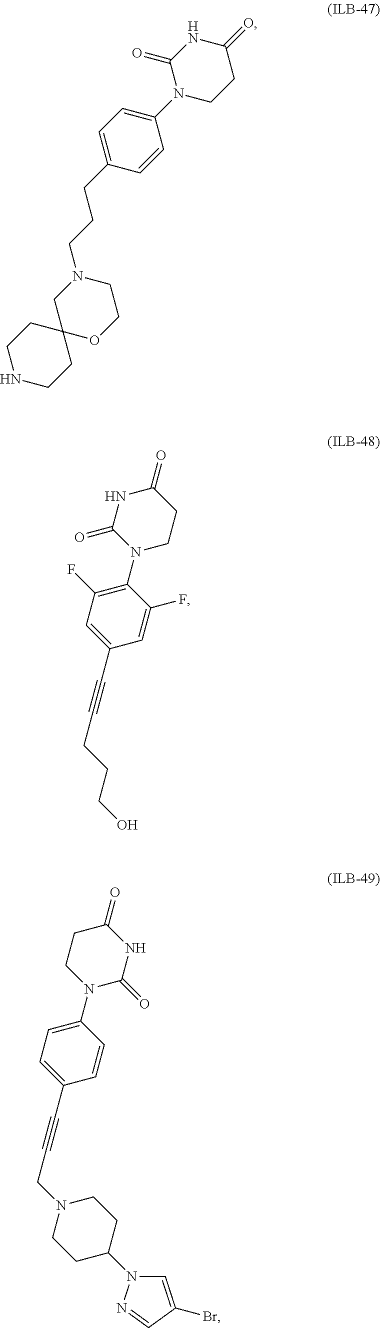 [SMILES: O=C1CCN(c2ccc(CCCN3CCOC4(CCNCC4)C3)cc2)C(=O)N1 ; O=C1CCN(c2c(F)cc(C#CCCCO)cc2F)C(=O)N1 ; O=C1CCN(c2ccc(C#CCN3CCC(n4cc(Br)cn4)CC3)cc2)C(=O)N1]

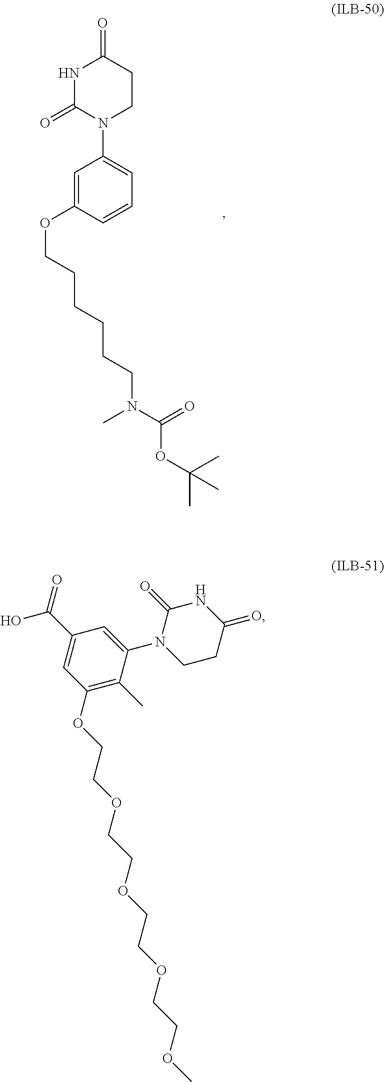 [SMILES: CN(CCCCCCOc1cccc(N2CCC(=O)NC2=O)c1)C(=O)OC(C)(C)C ; COCCOCCOCCOCCOc1cc(C(=O)O)cc(N2CCC(=O)NC2=O)c1C]

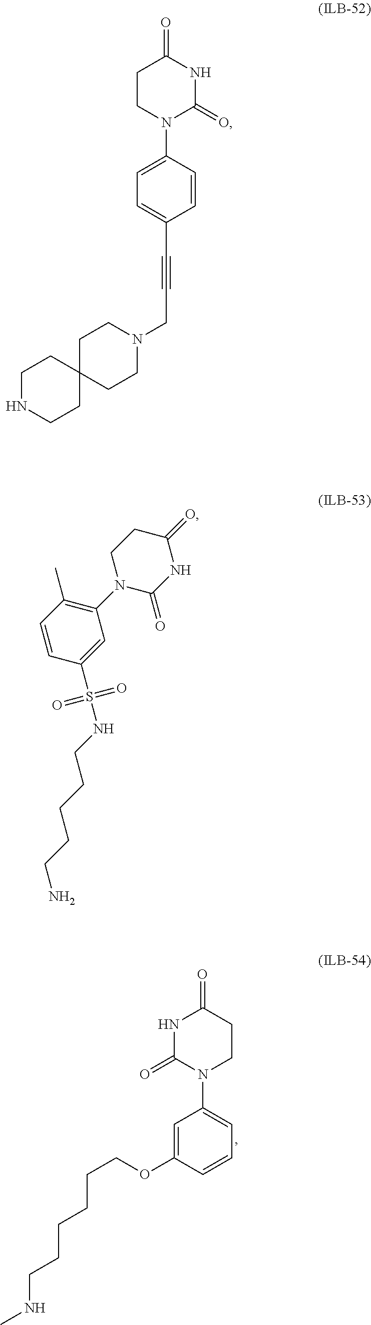 [SMILES: O=C1CCN(c2ccc(C#CCN3CCC4(CCNCC4)CC3)cc2)C(=O)N1 ; Cc1ccc(S(=O)(=O)NCCCCCN)cc1N1CCC(=O)NC1=O ; CNCCCCCCOc1cccc(N2CCC(=O)NC2=O)c1]

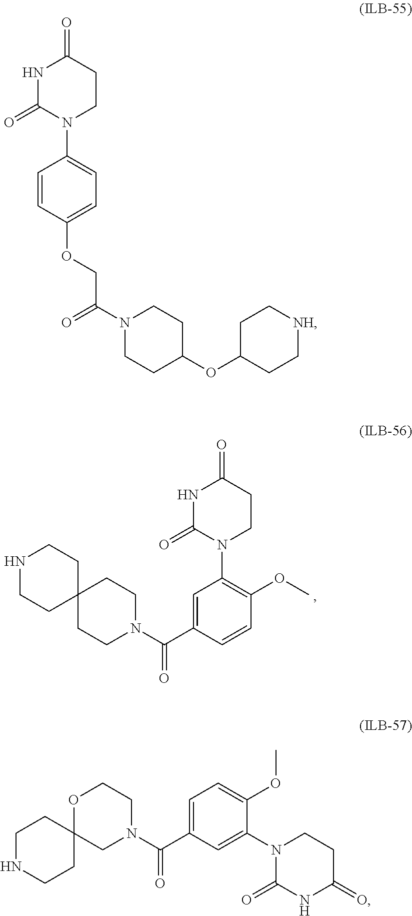 [SMILES: O=C1CCN(c2ccc(OCC(=O)N3CCC(OC4CCNCC4)CC3)cc2)C(=O)N1 ; COc1ccc(C(=O)N2CCC3(CCNCC3)CC2)cc1N1CCC(=O)NC1=O ; COc1ccc(C(=O)N2CCOC3(CCNCC3)C2)cc1N1CCC(=O)NC1=O]

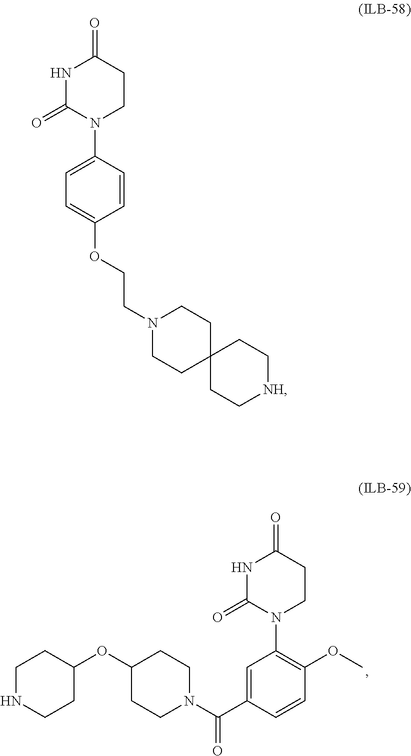 [SMILES: O=C1CCN(c2ccc(OCCN3CCC4(CCNCC4)CC3)cc2)C(=O)N1 ; COc1ccc(C(=O)N2CCC(OC3CCNCC3)CC2)cc1N1CCC(=O)NC1=O]

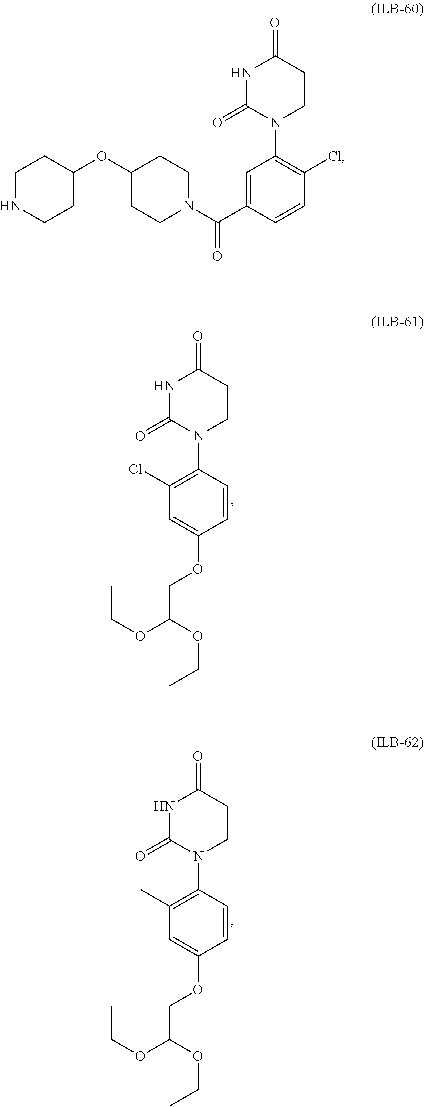 [SMILES: O=C1CCN(c2cc(C(=O)N3CCC(OC4CCNCC4)CC3)ccc2Cl)C(=O)N1 ; CCOC(COc1ccc(N2CCC(=O)NC2=O)c(Cl)c1)OCC ; CCOC(COc1ccc(N2CCC(=O)NC2=O)c(C)c1)OCC]

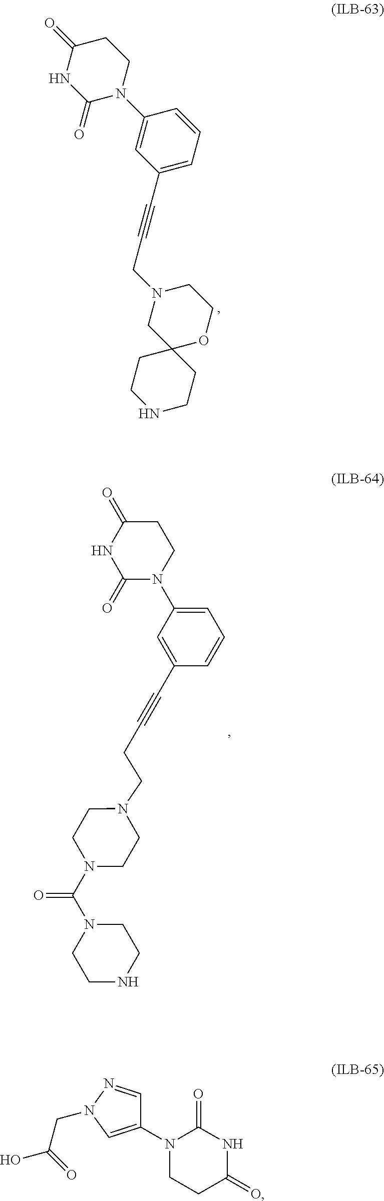 [SMILES: O=C1CCN(c2cccc(C#CCN3CCOC4(CCNCC4)C3)c2)C(=O)N1 ; O=C1CCN(c2cccc(C#CCCN3CCN(C(=O)N4CCNCC4)CC3)c2)C(=O)N1 ; O=C(O)Cn1cc(N2CCC(=O)NC2=O)cn1]

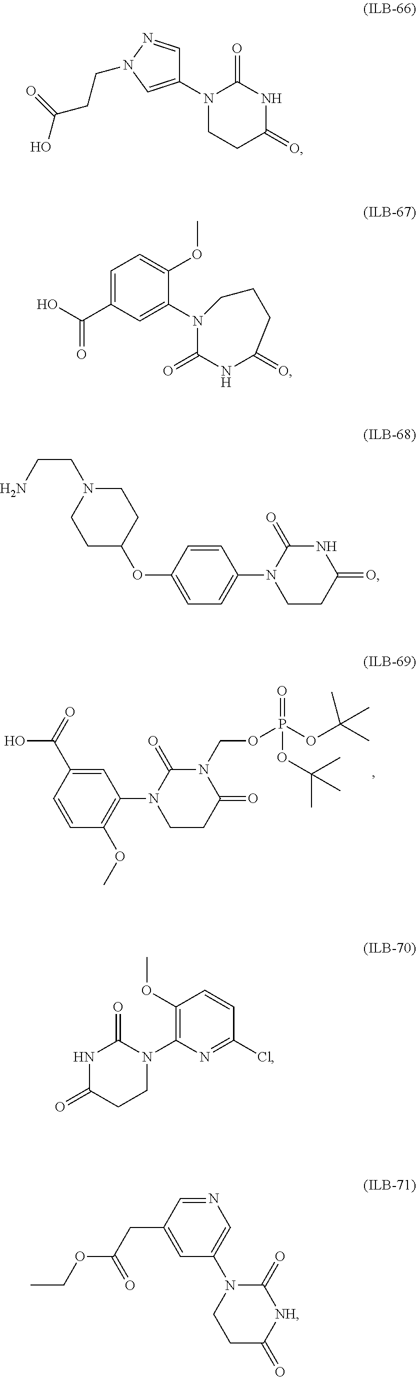

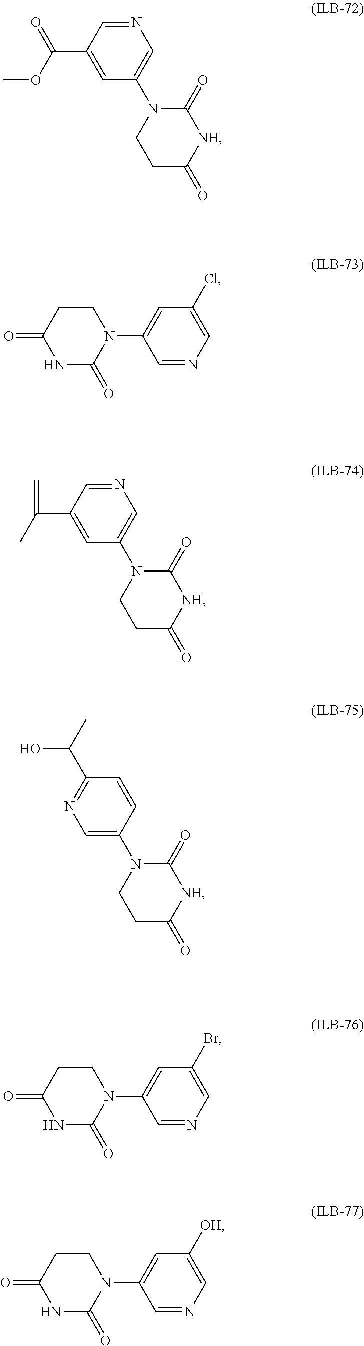 [SMILES: COC(=O)c1cncc(N2CCC(=O)NC2=O)c1 ; O=C1CCN(c2cncc(Cl)c2)C(=O)N1 ; C=C(C)c1cncc(N2CCC(=O)NC2=O)c1 ; CC(O)c1ccc(N2CCC(=O)NC2=O)cn1 ; O=C1CCN(c2cncc(Br)c2)C(=O)N1 ; O=C1CCN(c2cncc(O)c2)C(=O)N1]

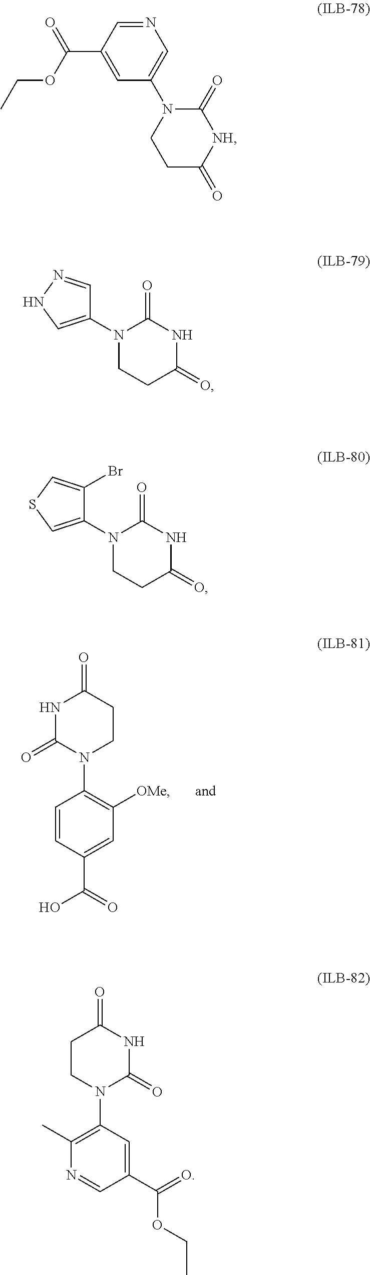 [SMILES: CCOC(=O)c1cncc(N2CCC(=O)NC2=O)c1 ; O=C1CCN(c2cn[nH]c2)C(=O)N1 ; O=C1CCN(c2cscc2Br)C(=O)N1 ; COc1cc(C(=O)O)ccc1N1CCC(=O)NC1=O ; CCOC(=O)c1cnc(C)c(N2CCC(=O)NC2=O)c1]

Another embodiment is a compound of Formula (ILB-IV):

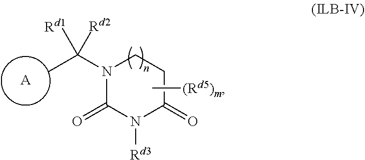

or a pharmaceutically acceptable salt, hydrate, solvate, prodrug, stereoisomer, or tautomer thereof, wherein:
Ring A is selected from the group consisting of

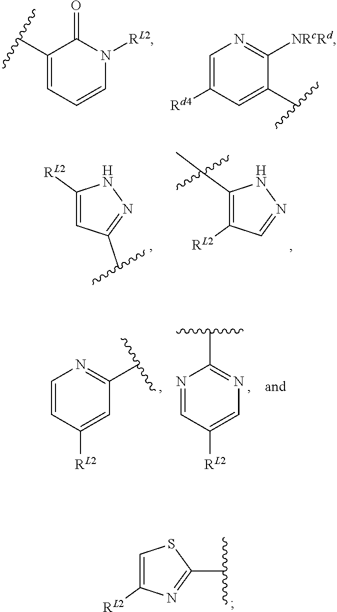

[symbol] denotes the point of attachment to the base molecule of (ILB-IV);
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, hydroxyl, oxo, polyethylene glycol (PEG), halogen, C₁₋₃ alkyl, C₃₋₆ cycloalkyl, C₁₋₃ alkoxyl, C₁₋₆ haloalkyl, C₁₋₆ heteroalkyl, and —OC₁₋₇ heteroalkyl;
each R^{d4a} is independently selected from the group consisting of H, polyethylene glycol (PEG), C₁₋₃ alkyl, C₃₋₆ cycloalkyl, C₂₋₆ haloalkyl, and C₂₋₆ heteroalkyl;
each R^{d5} is independently selected from the group consisting of H, C₁₋₆ alkyl, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
R^{L2} is selected from the group consisting of hydroxyl, halogen, C₂₋₆ alkyl, C₁₋₃ alkoxyl, C₂₋₆ alkenyl, C₂₋₆ alkynyl, C₂₋₆ hydroxyalkyl, —(CH₂)₂₋₆NHR^{c}, —(CH₂)₀₋₆NR^{c}R^{d}, —O—(CH₂)₂₋₆NHR^{c}, C₃₋₈ heteroalkyl, C₁₋₆ haloalkyl, —SO₂—NH—(CH₂)₂₋₆NHR^{c}, —(CH₂)₀₋₃C(O)OH, —O—(CH₂)₁₋₃C(O)H, —(CH₂)₁₋₃C(O)H, —O—(CH₂)₁₋₃C(O)OH, —(CH₂)₀₋₃C₃₋₇ carbocyclyl, —(CH₂)₀₋₃ heterocyclyl, —C(O)—(CH₂)₀₋₃ heterocyclyl, —O—(CH₂)₀₋₃ heterocyclyl, —C₂₋₆ alkynyl-heterocyclyl, —C₂₋₆ alkynyl-heterocyclyl-heteraryl, C₆ aryl, and heteroaryl, wherein the alkynyl, alkoxyl, heterocyclyl, heteroalkyl, carbocyclyl, aryl, and heteroaryl is substituted with 0-2 occurrences of halogen, hydroxyl, —(CH₂)₀₋₃C(O)H, —C(O)O-benzyl, —(CH₂)₂₋₆NHR^{c}, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, C₁₋₆ heteroalkyl, and —C(O)OC₁₋₆ alkyl;
R^{d} is H or C₁₋₄ alkyl; or R^{c} and R^{d} together with the nitrogen atom to which they are attached form a heterocyclyl substituted with 0-2 occurrences of —O-heterocyclyl,
R^{p} is H or C₁₋₆ alkyl;
m is 1 or 2; and
n is 1 or 2.

In an embodiment, ring A is selected from the group consisting of:

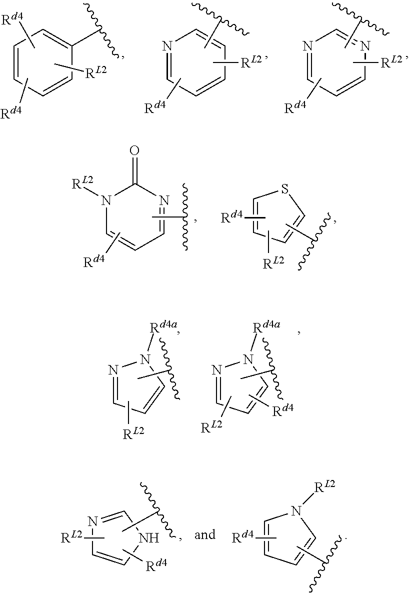

Another embodiment is a compound or a pharmaceutically acceptable salt, hydrate, solvate, prodrug, stereoisomer, or tautomer thereof, selected from:

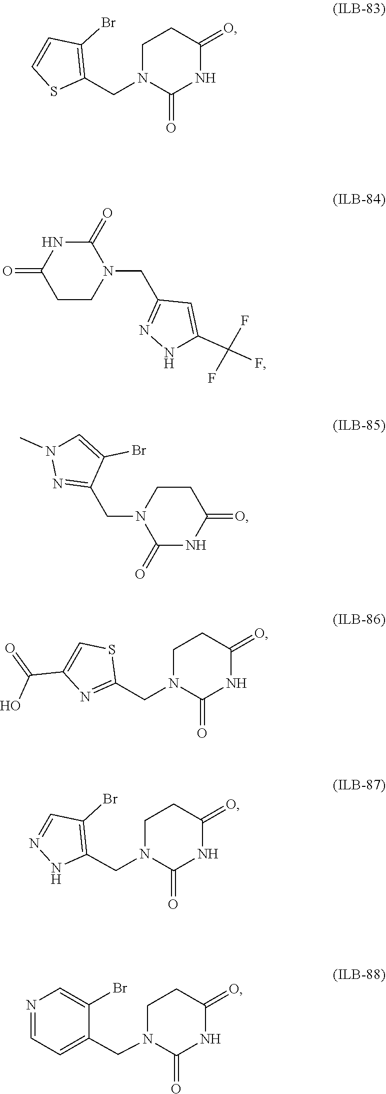 [SMILES: O=C1CCN(Cc2sccc2Br)C(=O)N1 ; O=C1CCN(Cc2cc(C(F)(F)F)[nH]n2)C(=O)N1 ; Cn1cc(Br)c(CN2CCC(=O)NC2=O)n1 ; O=C1CCN(Cc2nc(C(=O)O)cs2)C(=O)N1 ; O=C1CCN(Cc2[nH]ncc2Br)C(=O)N1 ; O=C1CCN(Cc2ccncc2Br)C(=O)N1]

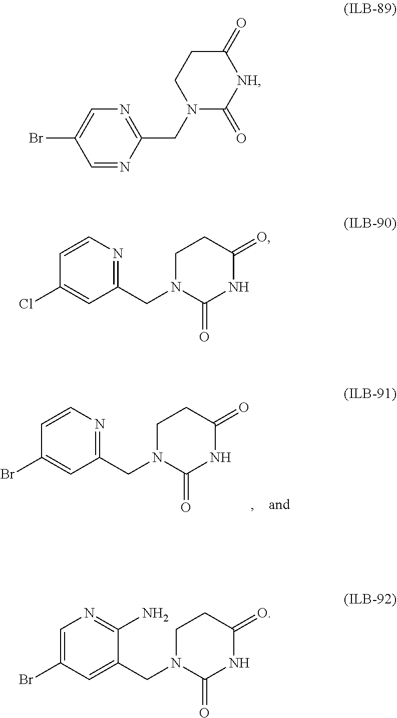 [SMILES: O=C1CCN(Cc2ncc(Br)cn2)C(=O)N1 ; O=C1CCN(Cc2cc(Cl)ccn2)C(=O)N1 ; O=C1CCN(Cc2cc(Br)ccn2)C(=O)N1 ; Nc1ncc(Br)cc1CN1CCC(=O)NC1=O]

Another embodiment is a bifunctional compound of Formula (II):

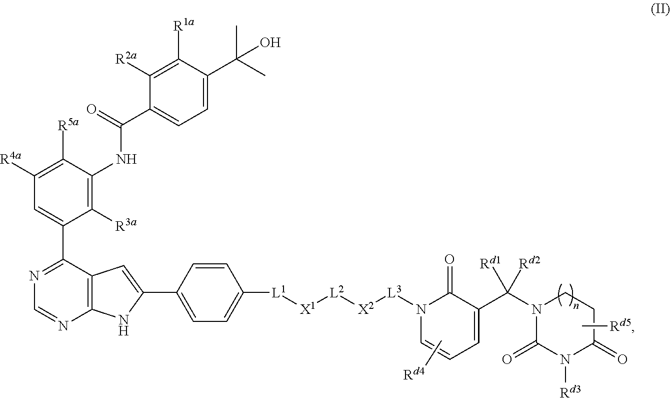

or a pharmaceutically acceptable salt, hydrate, solvate, prodrug, stereoisomer, or tautomer thereof, wherein:
R^{1a} is H or halo;
R^{2a} is halo;
R^{3a} is C₁₋₆ alkyl;
R^{4a} is halo;
R^{5a} is H or halo;
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-III);
wherein no more than 2 of L¹, X¹, X², L², and L³ can simultaneously be a bond;
R′ is hydrogen or C₁₋₆ alkyl;
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl; and
R^{p} is H or C₁₋₆ alkyl.

In an embodiment, R^{2a} is fluoro. In an embodiment, R^{3a} is C₁₋₃ alkyl. In an embodiment, R^{3a} is methyl.

In an embodiment, R^{4a} is fluoro. In an embodiment, L¹ is C₁₋₉ alkylene. In an embodiment, —X-L²-X²— is:

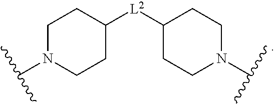

In an embodiment, L² is —C(O)—, —O—, or C₁₋₆ alkylene. In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, R^{d4} is H. In an embodiment, R^{d1} is H. In an embodiment, R^{d2} is H. In an embodiment, R^{d1} and R^{d2} are both H. In an embodiment, n is 1. In an embodiment, R^{d3} is H. In an embodiment, R^{d5} is H or C₁₋₃ alkyl.

In an embodiment, R^{d5} is H.

Another embodiment is a bifunctional compound of Formula (IIA):

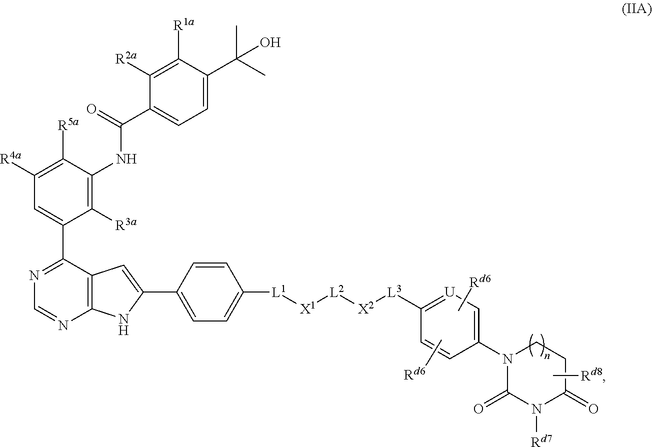

or a pharmaceutically acceptable salt, hydrate, solvate, prodrug, stereoisomer, or tautomer thereof, wherein:
R^{1a} is H or halo;
R^{2a} is halo;
R^{3a} is C₁₋₆ alkyl;
R^{4a} is halo;
R^{5a} is H or halo;
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-III);
wherein no more than 2 of L¹, X¹, X², L², and L³ can simultaneously be a bond;
R′ is hydrogen or C₁₋₆ alkyl;
U is —CR^{d6} or N;
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
U is —CR^{d6} or N;
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d8} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, R^{2a} is fluoro. In an embodiment, R^{3a} is C₁₋₃ alkyl. In an embodiment, R^{3a} is methyl. In an embodiment, R^{4a} is fluoro. In an embodiment, L¹ is C₁₋₉ alkylene. In an embodiment, —X¹-L²-X²— is:

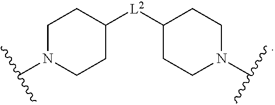

In an embodiment, L² is —C(O)—, —O—, or C₁₋₆ alkylene. In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, R^{d4} is H. In an embodiment, R^{d1} is H. In an embodiment, R^{d2} is H. In an embodiment, R^{d1} and R^{d2} are both H. In an embodiment, n is 1. In an embodiment, R^{d3} is H. In an embodiment, R^{d5} is H or C₁₋₃ alkyl. In an embodiment, R^{d5} is H.

Another embodiment is a bifunctional compound, or a pharmaceutically acceptable salt, hydrate, solvate, prodrug, stereoisomer, or tautomer thereof, selected from:

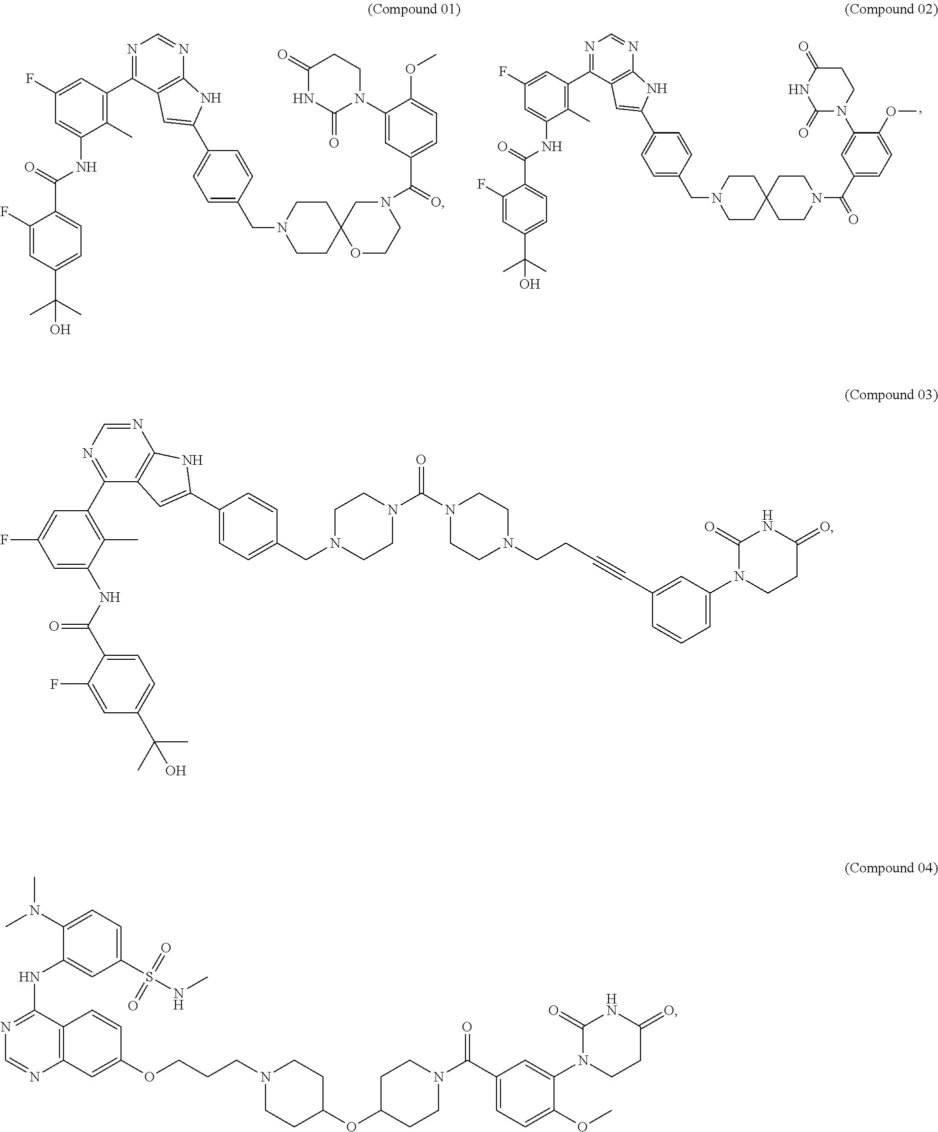 [SMILES: COc1ccc(C(=O)N2CCOC3(CCN(Cc4ccc(-c5cc6c(-c7cc(F)cc(NC(=O)c8ccc(C(C)(C)O)cc8F)c7C)ncnc6[nH]5)cc4)CC3)C2)cc1N1CCC(=O)NC1=O ; COc1ccc(C(=O)N2CCC3(CCN(Cc4ccc(-c5cc6c(-c7cc(F)cc(NC(=O)c8ccc(C(C)(C)O)cc8F)c7C)ncnc6[nH]5)cc4)CC3)CC2)cc1N1CCC(=O)NC1=O ; Cc1c(NC(=O)c2ccc(C(C)(C)O)cc2F)cc(F)cc1-c1ncnc2[nH]c(-c3ccc(CN4CCN(C(=O)N5CCN(CCC#Cc6cccc(N7CCC(=O)NC7=O)c6)CC5)CC4)cc3)cc12 ; CNS(=O)(=O)c1ccc(N(C)C)c(Nc2ncnc3cc(OCCCN4CCC(OC5CCN(C(=O)c6ccc(OC)c(N7CCC(=O)NC7=O)c6)CC5)CC4)ccc23)c1]

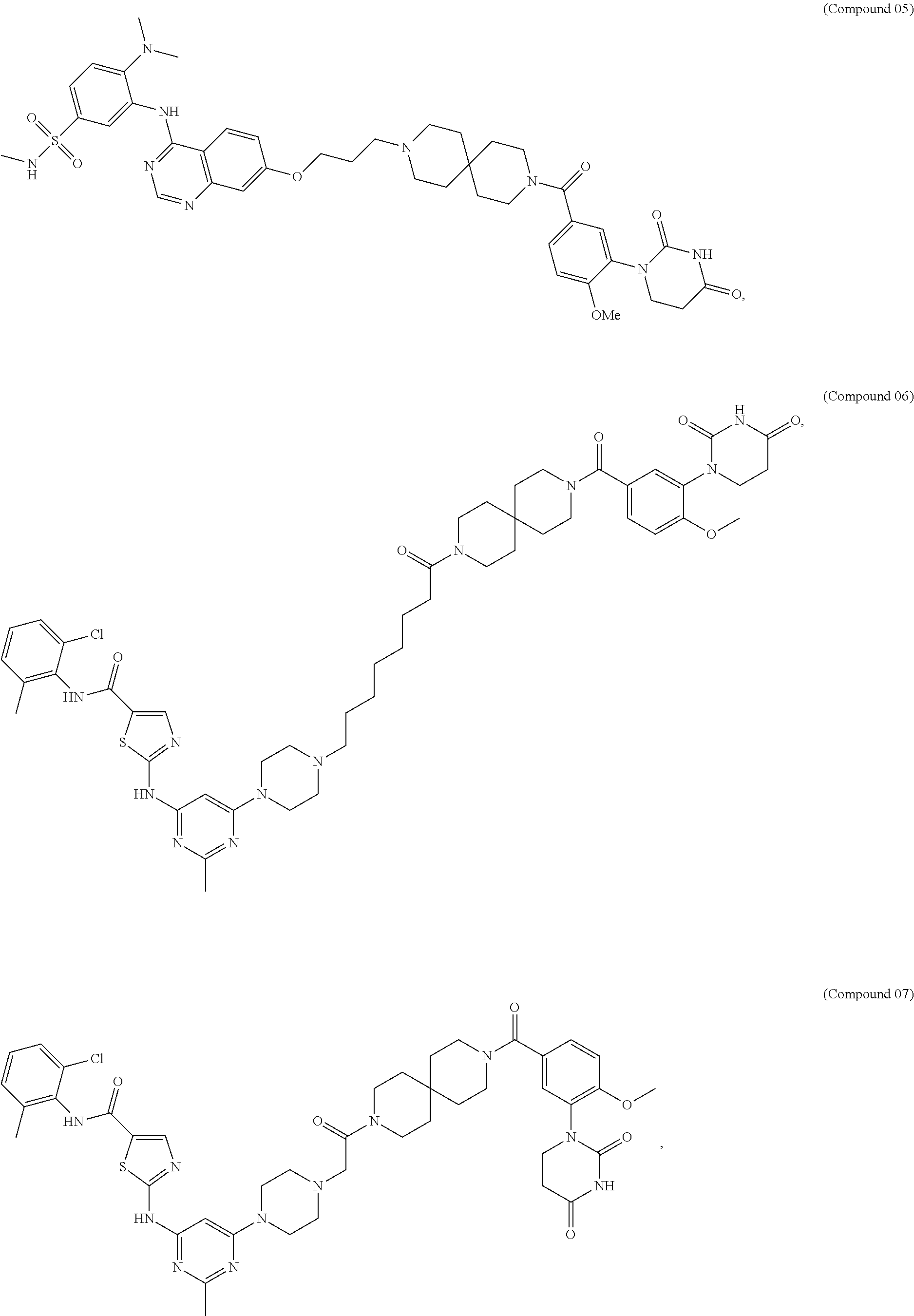 [SMILES: CNS(=O)(=O)c1ccc(N(C)C)c(Nc2ncnc3cc(OCCCN4CCC5(CC4)CCN(C(=O)c4ccc(OC)c(N6CCC(=O)NC6=O)c4)CC5)ccc23)c1 ; COc1ccc(C(=O)N2CCC3(CCN(C(=O)CCCCCCCN4CCN(c5cc(Nc6ncc(C(=O)Nc7c(C)cccc7Cl)s6)nc(C)n5)CC4)CC3)CC2)cc1N1CCC(=O)NC1=O ; COc1ccc(C(=O)N2CCC3(CCN(C(=O)CN4CCN(c5cc(Nc6ncc(C(=O)Nc7c(C)cccc7Cl)s6)nc(C)n5)CC4)CC3)CC2)cc1N1CCC(=O)NC1=O]

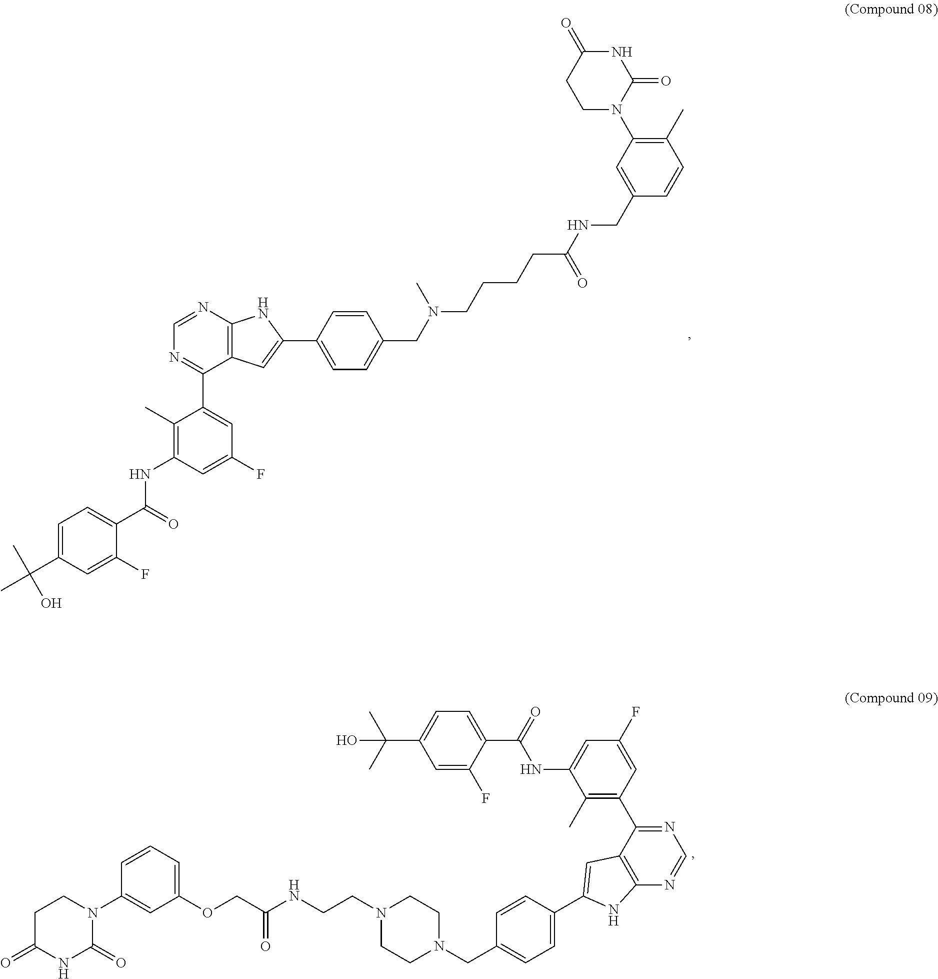 [SMILES: Cc1ccc(CNC(=O)CCCCN(C)Cc2ccc(-c3cc4c(-c5cc(F)cc(NC(=O)c6ccc(C(C)(C)O)cc6F)c5C)ncnc4[nH]3)cc2)cc1N1CCC(=O)NC1=O ; Cc1c(NC(=O)c2ccc(C(C)(C)O)cc2F)cc(F)cc1-c1ncnc2[nH]c(-c3ccc(CN4CCN(CCNC(=O)COc5cccc(N6CCC(=O)NC6=O)c5)CC4)cc3)cc12]

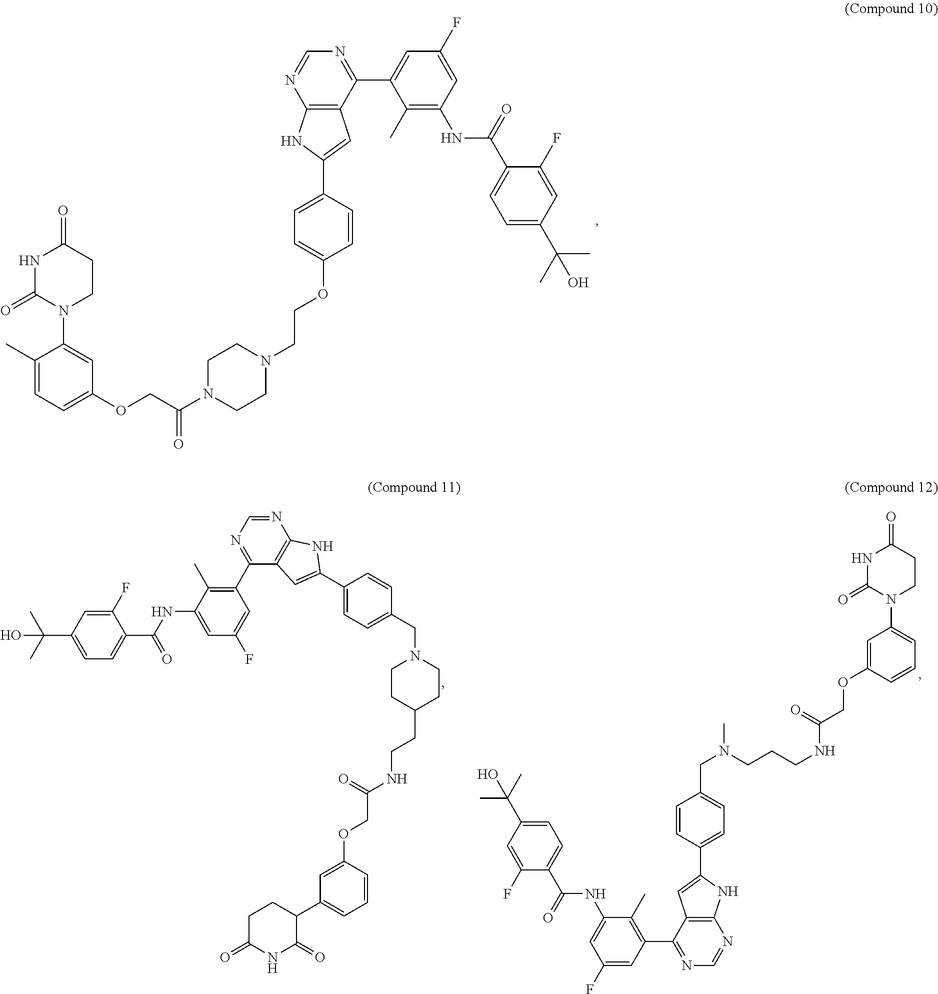

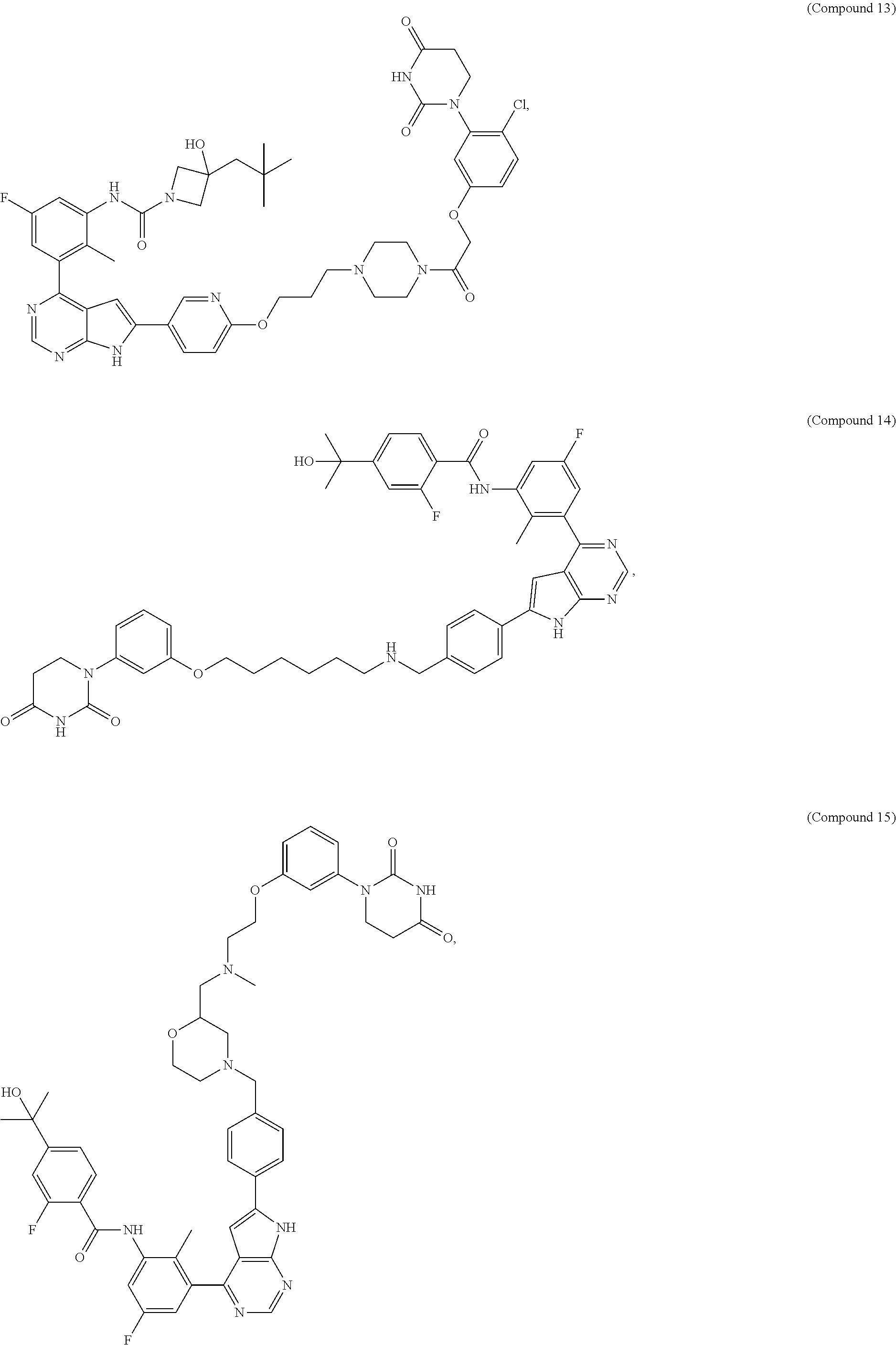 [SMILES: Cc1c(NC(=O)N2CC(O)(CC(C)(C)C)C2)cc(F)cc1-c1ncnc2[nH]c(-c3ccc(OCCCN4CCN(C(=O)COc5ccc(Cl)c(N6CCC(=O)NC6=O)c5)CC4)nc3)cc12 ; Cc1c(NC(=O)c2ccc(C(C)(C)O)cc2F)cc(F)cc1-c1ncnc2[nH]c(-c3ccc(CNCCCCCCOc4cccc(N5CCC(=O)NC5=O)c4)cc3)cc12 ; Cc1c(NC(=O)c2ccc(C(C)(C)O)cc2F)cc(F)cc1-c1ncnc2[nH]c(-c3ccc(CN4CCOC(CN(C)CCOc5cccc(N6CCC(=O)NC6=O)c5)C4)cc3)cc12]

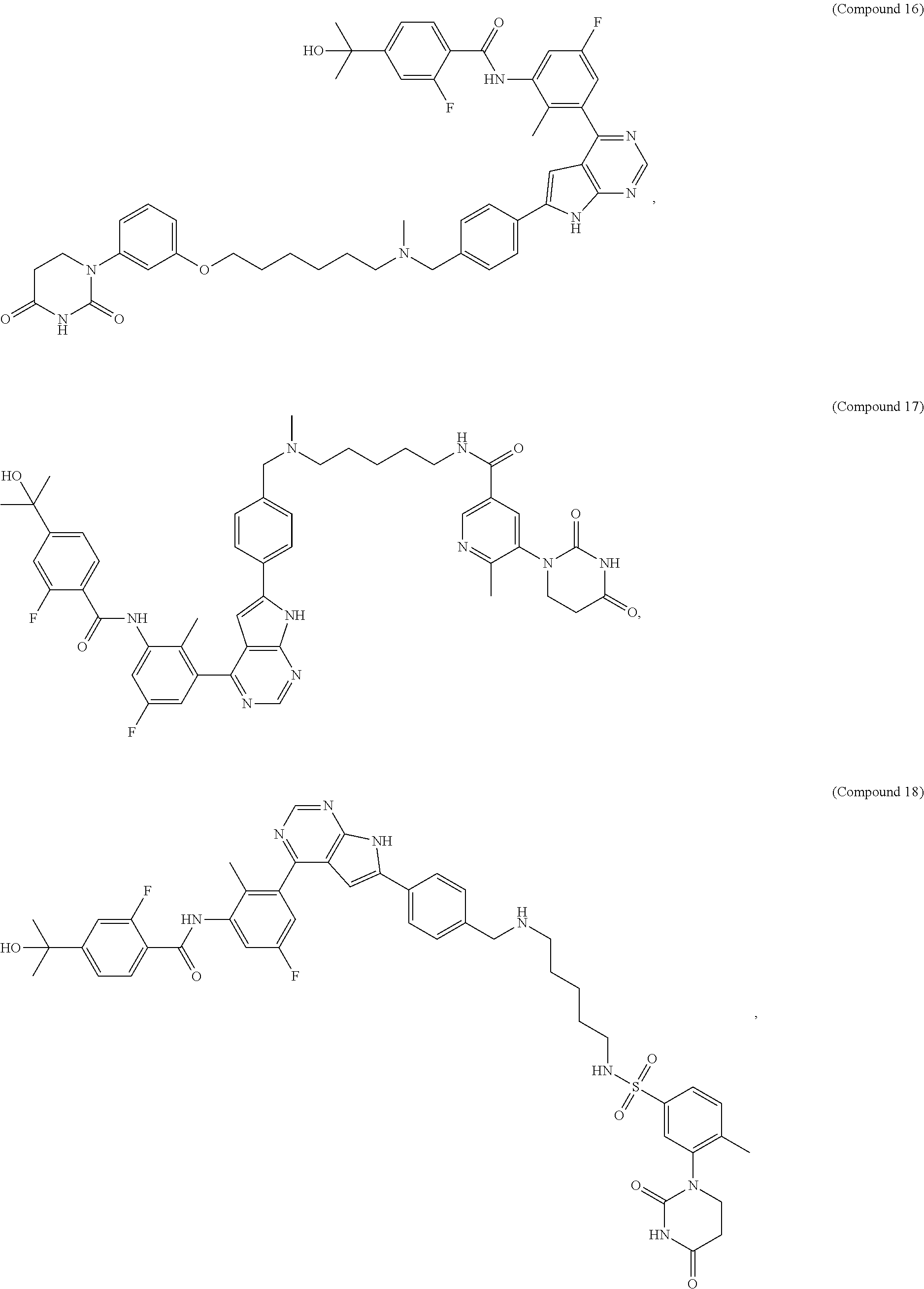 [SMILES: Cc1c(NC(=O)c2ccc(C(C)(C)O)cc2F)cc(F)cc1-c1ncnc2[nH]c(-c3ccc(CN(C)CCCCCCOc4cccc(N5CCC(=O)NC5=O)c4)cc3)cc12 ; Cc1ncc(C(=O)NCCCCCN(C)Cc2ccc(-c3cc4c(-c5cc(F)cc(NC(=O)c6ccc(C(C)(C)O)cc6F)c5C)ncnc4[nH]3)cc2)cc1N1CCC(=O)NC1=O ; Cc1ccc(S(=O)(=O)NCCCCCNCc2ccc(-c3cc4c(-c5cc(F)cc(NC(=O)c6ccc(C(C)(C)O)cc6F)c5C)ncnc4[nH]3)cc2)cc1N1CCC(=O)NC1=O]

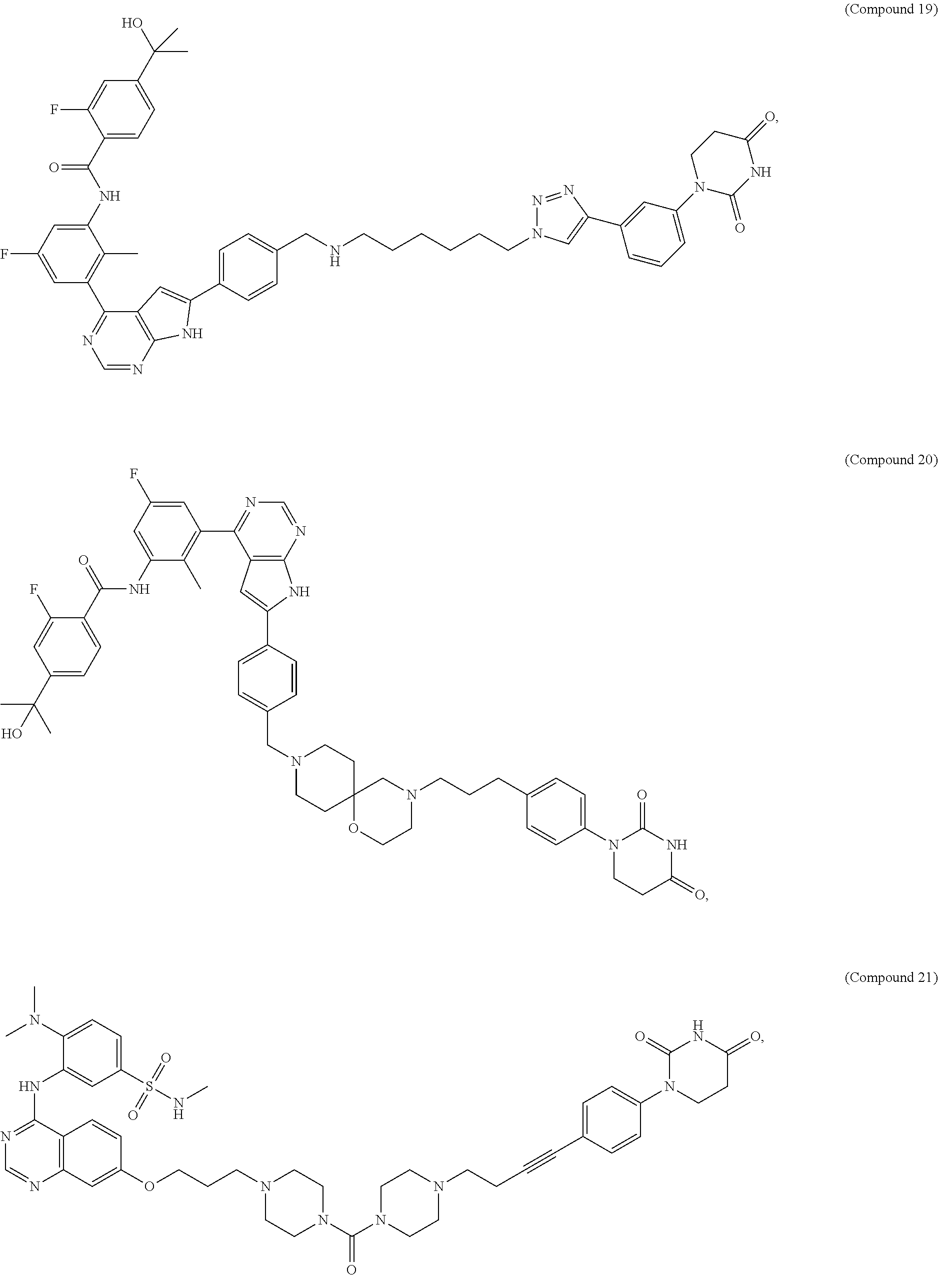 [SMILES: Cc1c(NC(=O)c2ccc(C(C)(C)O)cc2F)cc(F)cc1-c1ncnc2[nH]c(-c3ccc(CNCCCCCCn4cc(-c5cccc(N6CCC(=O)NC6=O)c5)nn4)cc3)cc12 ; Cc1c(NC(=O)c2ccc(C(C)(C)O)cc2F)cc(F)cc1-c1ncnc2[nH]c(-c3ccc(CN4CCC5(CC4)CN(CCCc4ccc(N6CCC(=O)NC6=O)cc4)CCO5)cc3)cc12 ; CNS(=O)(=O)c1ccc(N(C)C)c(Nc2ncnc3cc(OCCCN4CCN(C(=O)N5CCN(CCC#Cc6ccc(N7CCC(=O)NC7=O)cc6)CC5)CC4)ccc23)c1]

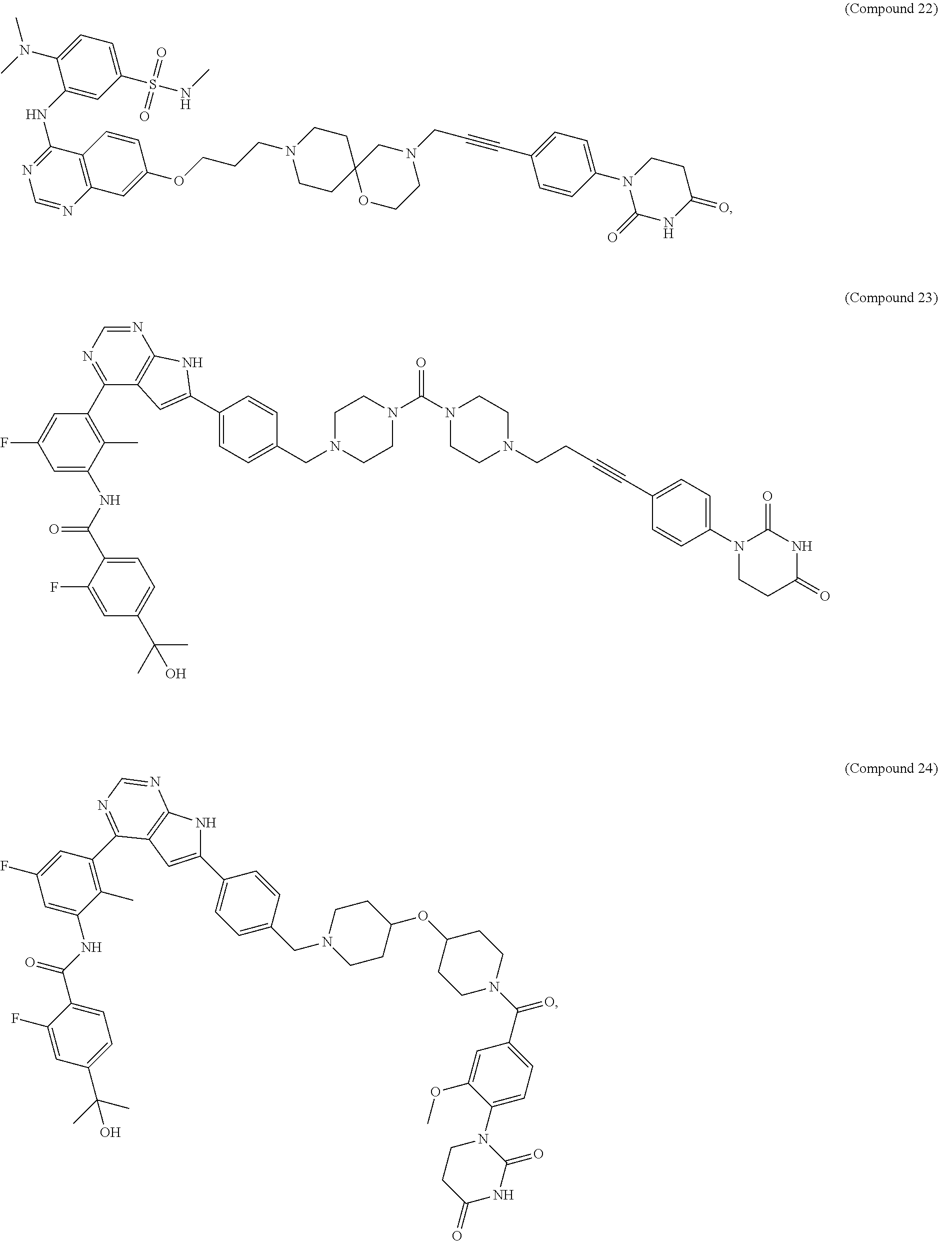 [SMILES: CNS(=O)(=O)c1ccc(N(C)C)c(Nc2ncnc3cc(OCCCN4CCC5(CC4)CN(CC#Cc4ccc(N6CCC(=O)NC6=O)cc4)CCO5)ccc23)c1 ; Cc1c(NC(=O)c2ccc(C(C)(C)O)cc2F)cc(F)cc1-c1ncnc2[nH]c(-c3ccc(CN4CCN(C(=O)N5CCN(CCC#Cc6ccc(N7CCC(=O)NC7=O)cc6)CC5)CC4)cc3)cc12 ; COc1cc(C(=O)N2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O]

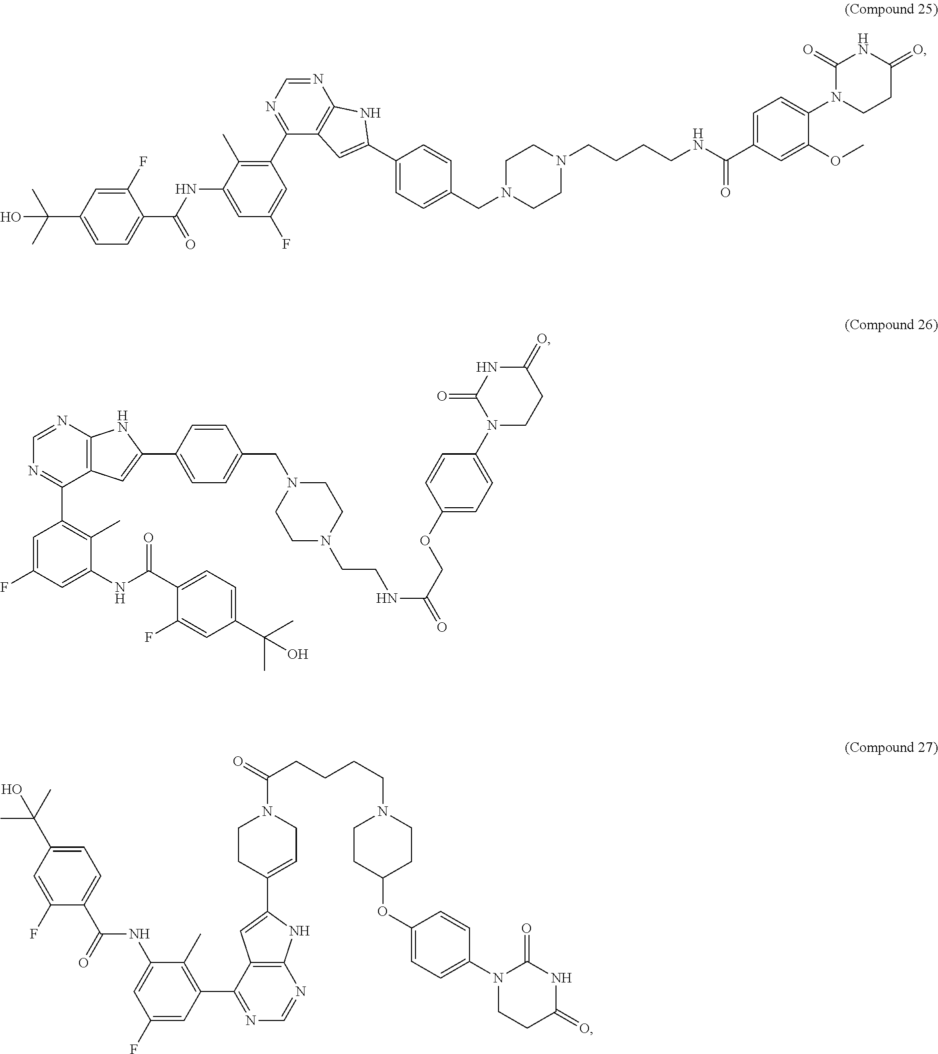 [SMILES: COc1cc(C(=O)NCCCCN2CCN(Cc3ccc(-c4cc5c(-c6cc(F)cc(NC(=O)c7ccc(C(C)(C)O)cc7F)c6C)ncnc5[nH]4)cc3)CC2)ccc1N1CCC(=O)NC1=O ; Cc1c(NC(=O)c2ccc(C(C)(C)O)cc2F)cc(F)cc1-c1ncnc2[nH]c(-c3ccc(CN4CCN(CCNC(=O)COc5ccc(N6CCC(=O)NC6=O)cc5)CC4)cc3)cc12 ; Cc1c(NC(=O)c2ccc(C(C)(C)O)cc2F)cc(F)cc1-c1ncnc2[nH]c(C3=CCN(C(=O)CCCCN4CCC(Oc5ccc(N6CCC(=O)NC6=O)cc5)CC4)CC3)cc12]

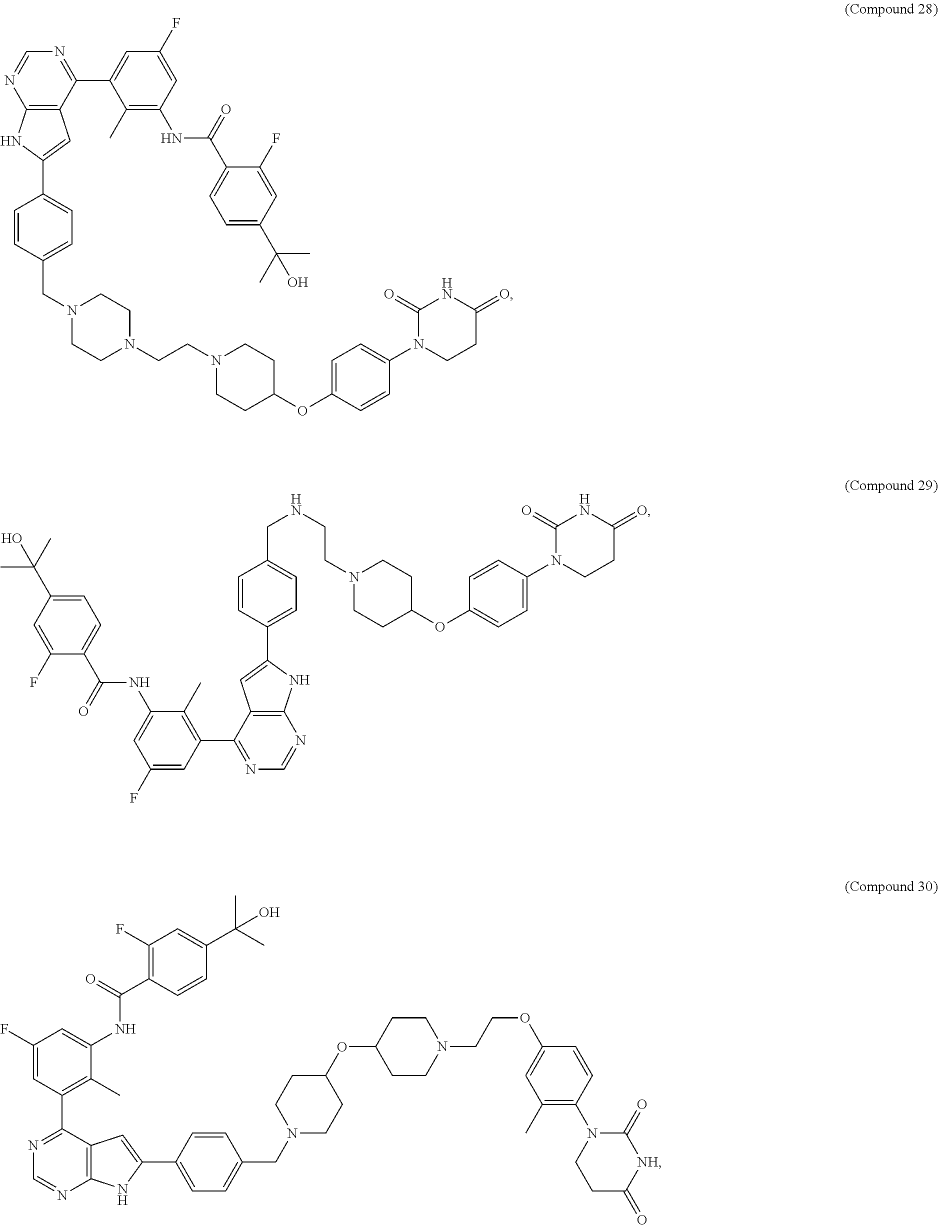 [SMILES: Cc1c(NC(=O)c2ccc(C(C)(C)O)cc2F)cc(F)cc1-c1ncnc2[nH]c(-c3ccc(CN4CCN(CCN5CCC(Oc6ccc(N7CCC(=O)NC7=O)cc6)CC5)CC4)cc3)cc12 ; Cc1c(NC(=O)c2ccc(C(C)(C)O)cc2F)cc(F)cc1-c1ncnc2[nH]c(-c3ccc(CNCCN4CCC(Oc5ccc(N6CCC(=O)NC6=O)cc5)CC4)cc3)cc12 ; Cc1cc(OCCN2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O]

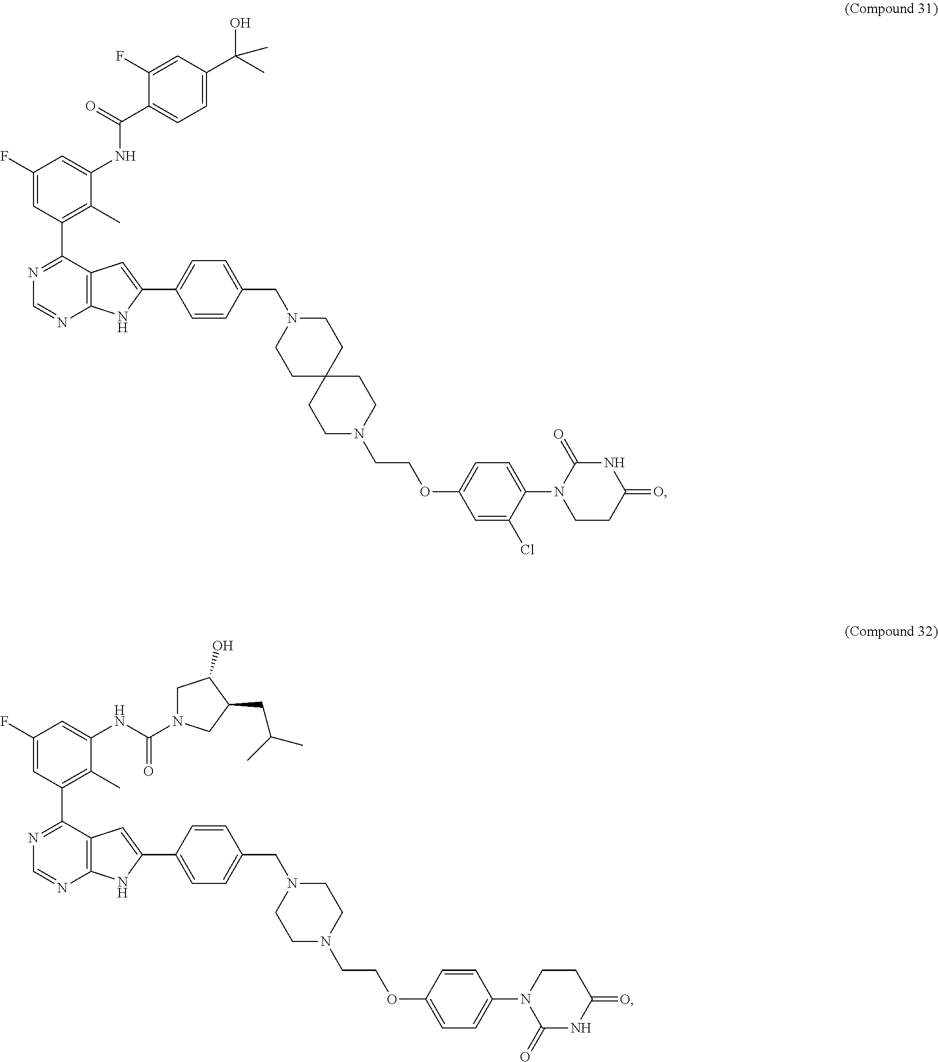 [SMILES: Cc1c(NC(=O)c2ccc(C(C)(C)O)cc2F)cc(F)cc1-c1ncnc2[nH]c(-c3ccc(CN4CCC5(CCN(CCOc6ccc(N7CCC(=O)NC7=O)c(Cl)c6)CC5)CC4)cc3)cc12 ; Cc1c(NC(=O)N2C[C@H](CC(C)C)[C@@H](O)C2)cc(F)cc1-c1ncnc2[nH]c(-c3ccc(CN4CCN(CCOc5ccc(N6CCC(=O)NC6=O)cc5)CC4)cc3)cc12]

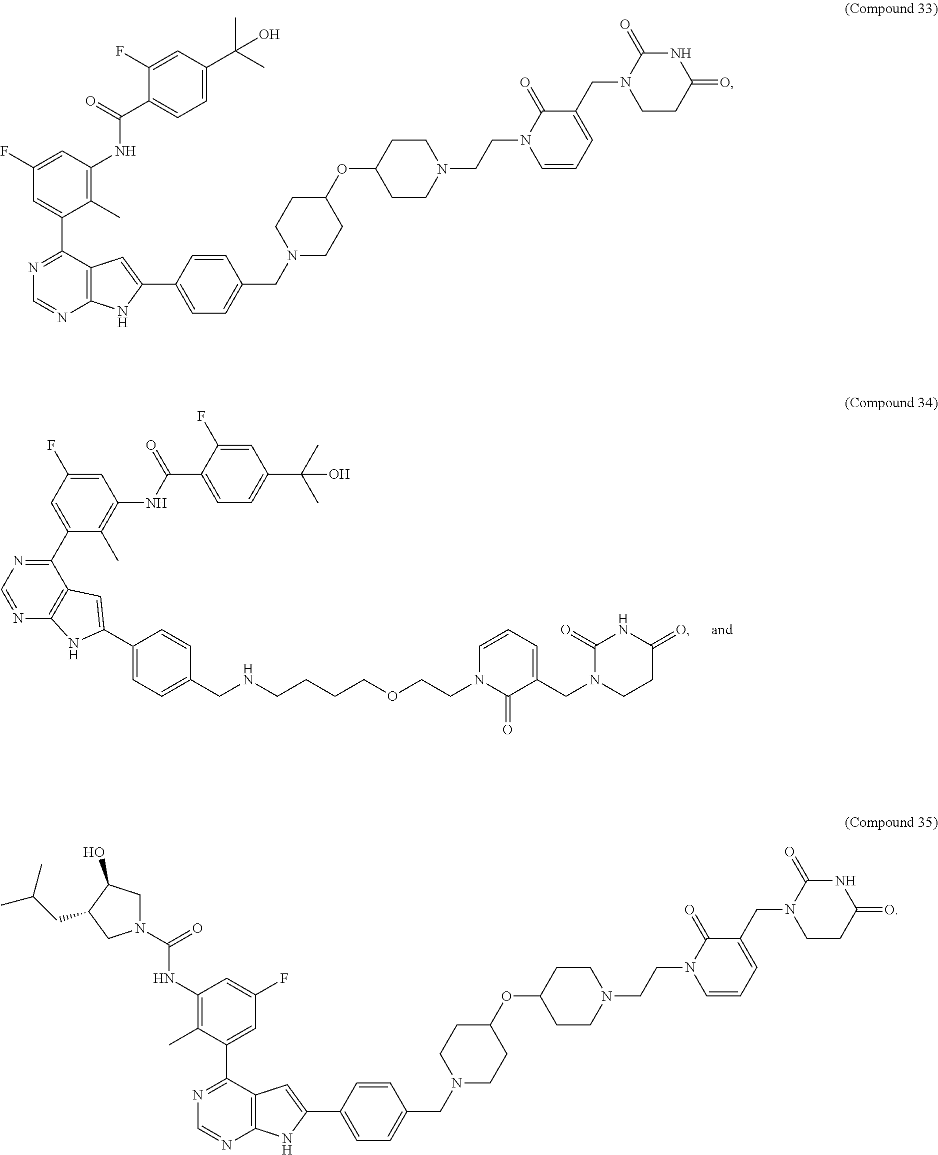 [SMILES: Cc1c(NC(=O)c2ccc(C(C)(C)O)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12 ; Cc1c(NC(=O)c2ccc(C(C)(C)O)cc2F)cc(F)cc1-c1ncnc2[nH]c(-c3ccc(CNCCCCOCCn4cccc(CN5CCC(=O)NC5=O)c4=O)cc3)cc12 ; Cc1c(NC(=O)N2C[C@H](CC(C)C)[C@@H](O)C2)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

Another embodiment is a pharmaceutical composition comprising any of the compounds described herein, or a pharmaceutically acceptable salt, hydrate, solvate, prodrug, stereoisomer, or tautomer thereof, and a pharmaceutically acceptable carrier.

Another embodiment is a pharmaceutical combination comprising any of the compounds described herein, or a pharmaceutically acceptable salt, hydrate, solvate, prodrug, stereoisomer, or tautomer thereof, and a therapeutic agent.

Another embodiment is a method of treating a respiratory disorder, a proliferative disorder, an autoimmune disorder, an autoinflammatory disorder, an inflammatory disorder, a neurological disorder, and an infectious disease or disorder in a subject in need thereof, the method comprising administering to the subject a therapeutically effective amount of a compound of any one of the preceding claims, or a pharmaceutically acceptable salt thereof. In an embodiment, the disorder is a proliferative disorder. In an embodiment, the proliferative disorder is cancer.

Another embodiment is the use of a compound of any one of the preceding claims, or a pharmaceutically acceptable salt, hydrate, solvate, prodrug, stereoisomer, or tautomer thereof in the preparation of a medicament for treating a respiratory disorder, a proliferative disorder, an autoimmune disorder, an autoinflammatory disorder, an inflammatory disorder, a neurological disorder, and an infectious disease or disorder in a subject in need thereof. One aspect is se of a compound of any one of the preceding claims, or a pharmaceutically acceptable salt, hydrate, solvate, prodrug, stereoisomer, or tautomer thereof for treating cancer.

### BRIEF DESCRIPTION OF THE DRAWINGS

FIG. 1 depicts a schematic of a bifunctional compound, such as a compound disclosed herein, which is bound to a protein of interest (POI), and which has recruited the POI to the E3 Ubiquitin ligase binding complex for tagging with Ubiquitin (Ub), marking the POI for degradation by the ligase, followed by translocation to the proteasome and subsequent degradation

FIG. 2 depicts a scheme for in silico design of bifunctional degraders. “B” is a hypothetical bifunctional degrader with targeting motifs for the target protein (a) and the E3 ligase substrate receptor (c). Curved arrows on “B” depict conformational degrees of rotation. “A” depicts a target protein. “C” depicts the E3 ligase substrate receptor.

FIG. 3A shows a Hill plot of TNNI3K expression as a function of compound 22 concentration.

FIG. 3B shows a bar graph of TNNI3K expression as a function of compound 22 concentration.

FIG. 3C shows a Hill plot of TNNI3K expression as a function of compound 21 concentration.

FIG. 3D shows a bar graph of TNNI3K expression as a function of compound 21 concentration.

FIG. 3E shows volcano plots depicting the identification of degrader-dependent CRBN substrate candidates. HEK293 and TMD8 cells were treated with 1 μM dasatinib, 1 μM compound 06, 1 μM compound 07 or DMSO and protein abundance was analyzed using TMT quantification mass spectrometry. Significant changes were assessed by limma, log 2 fold changes are shown on the x-axis and p-values on the y-axis. Proteins with kinase annotations in UniProt are shown as squares and kinases with a log₂ fold change≤−0.6 and a p-value≤0.01 are labeled with the corresponding gene name.

FIG. 4A shows a Western blot of TNNI3K expression as a function of compound 22 concentration. β-actin is used as a control.

FIG. 4B shows a Western blot of TNNI3K expression as a function of compound 21 concentration. β-actin is used as a control.

### DETAILED DESCRIPTION

Described herein are compounds or a pharmaceutically acceptable salt, hydrate, solvate, prodrug, stereoisomer, or tautomer thereof that function to recruit targeted proteins to E3 ubiquitin ligase for degradation, methods of preparation thereof, and uses thereof.

In one aspect, the disclosure provides are compounds or a pharmaceutically acceptable salt, hydrate, solvate, prodrug, stereoisomer, or tautomer thereof, which recruit a targeted protein, as a bromodomain-containing protein or a protein kinase, to E3 ubiquitin ligase for degradation. In an embodiment, the compound or a pharmaceutically acceptable salt, hydrate, solvate, prodrug, stereoisomer, or tautomer thereof, is a compound of Formula (I):

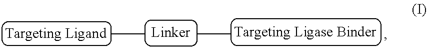

or a pharmaceutically acceptable salt, hydrate, solvate, prodrug, stereoisomer, or tautomer thereof, wherein:
the Targeting Ligand is a group that is capable of binding to a Target Protein;
the Linker is a group that covalently links the Targeting Ligand to the Targeting Ligase Binder; and
the Targeting Ligase Binder is a group that is capable of binding to a ligase (e.g., Cereblon E3 Ubiquitin ligase).

### Target Proteins

In one aspect, the disclosure provides compounds or a pharmaceutically acceptable salt, hydrate, solvate, prodrug, stereoisomer, or tautomer thereof, which recruit a targeted protein, such as a bromodomain-containing protein or a protein kinase, to E3 ubiquitin ligase for degradation. In an embodiment, the target protein is selected from Table 1 or Table 2.

### Targeting Ligands

The Targeting Ligand is a small molecule moiety that is capable of binding to a target protein or protein of interest (POI). In an embodiment, the target protein or POI is a target protein selected from Table 1. In an embodiment, the target protein or POI is a fusion protein. In an embodiment, the target protein or POI is a target protein selected from Table 2.

In an embodiment, the Targeting Ligand is a BRD9 targeting ligand of Formula (BRD9-I):

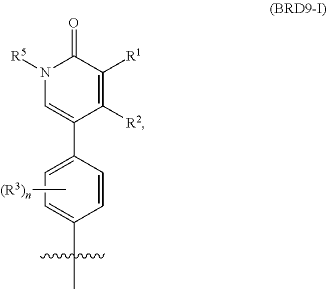

or a pharmaceutically acceptable salt, hydrate, solvate, prodrug, stereoisomer, or tautomer thereof, wherein:
R¹ and R² are independently selected from the group consisting of hydrogen and C₁₋₆ alkyl; or R¹ and R² together with the atoms to which they are attached form an aryl or heteroaryl;
R³ are each independently selected from the group consisting of C₁₋₆ alkyl, C₁₋₆ alkoxyl, and halogen;
R⁵ is selected from the group consisting of hydrogen and C₁₋₃ alkyl;
n is 0, 1, or 2.

In an embodiment, the Targeting Ligand is a BTK targeting ligand of Formula (BTK-I):

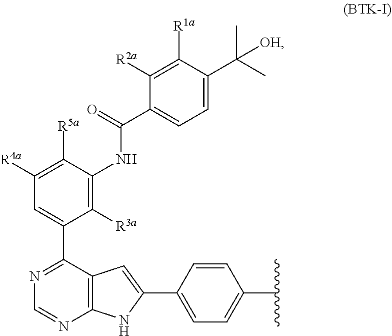

or a pharmaceutically acceptable salt, hydrate, solvate, prodrug, stereoisomer, or tautomer thereof, wherein:
R^{1a} is H or halo;
R^{2a} is halo;
R^{3a} is C₁₋₆ alkyl;
R^{4a} is halo; and
R^{5a} is H or halo.

Additional exemplary Targeting Ligands include, but are not limited to, the targeting ligands in Table 3:

TABLE 3
Exemplary Targeting Ligands

| Gene | Targeting Ligand |
|---|---|
| ALK | ceritinib |
| AR | 7a-methyl-19-nortestosterone |
| AR | Testosterone |
| BCR-ABL | dasatinib |
| BRD4 | JQ1 |
| BRD9 | BI-7273 |
| BTK | bosutinib |
| BTK | RN486 |
| CDK9 | SNS-032 |
| c-Met | foretinib |
| EGFR | AC480 |
| EGFR | ARRY-334543 |
| EGFR | afatinib |
| ERα | 4-OHT |
| EGFR Δ Exon 19 | gefitinib |
| FKBP12 | Steel Factor |
| FLT3 | 4SC-202 |
| FLT3 | Linifanib |
| FLT3 | Quizartinib (AC220) |
| JAK2 | AC430 |
| HDAC4 | AR-42 |
| HDAC6 | ACY-1215 |
| HDAC6 | AR-42 |
| TRIM24 | IACS-7e |

wherein the Targeting Ligand is attached to the Linker-Targeting Ligase Binder, e.g.,

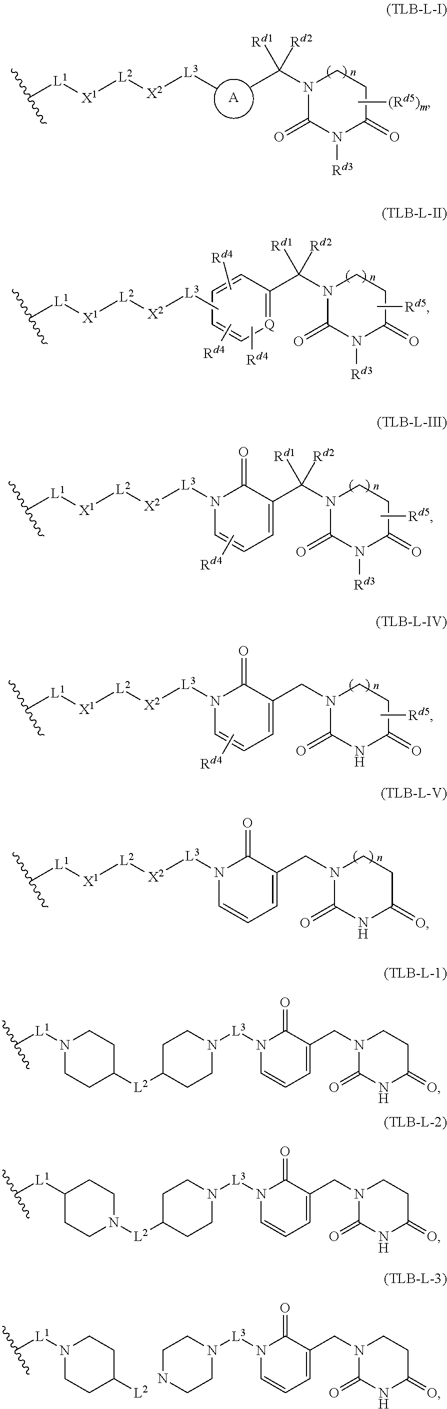

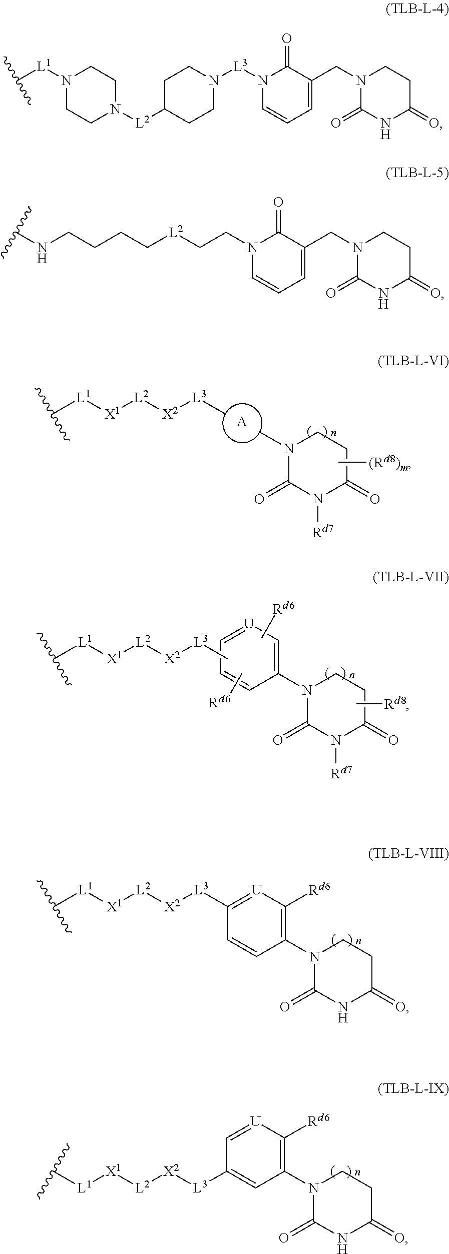

through a modifiable carbon, oxygen, nitrogen or sulfur atom on the Targeting Ligand.

In an embodiment, the Targeting Ligand is a targeting ligand described in Huang et al., “A Chemoproteomic Approach to Query the Degradable Kinome Using a Multi-kinase Degrader,” Cell Chem. Biol. 25(1): 88-99 (2018); An and Fu, “Small-molecule PROTACs: An emerging and promising approach for the development of targeted therapy drugs,” EBioMedicine 36: 553-562 (2018); Pei et al., “Small molecule PROTACs: an emerging technology for targeted therapy in drug discovery,” RSC Adv. 9:16967-16976 (2019); and Zou et al., Cell Biochem. Funct. 37: 21-30 (2019), each of which is incorporated by reference herein in its entirety.

In an embodiment, the Targeting Ligand is selected from the group consisting of:

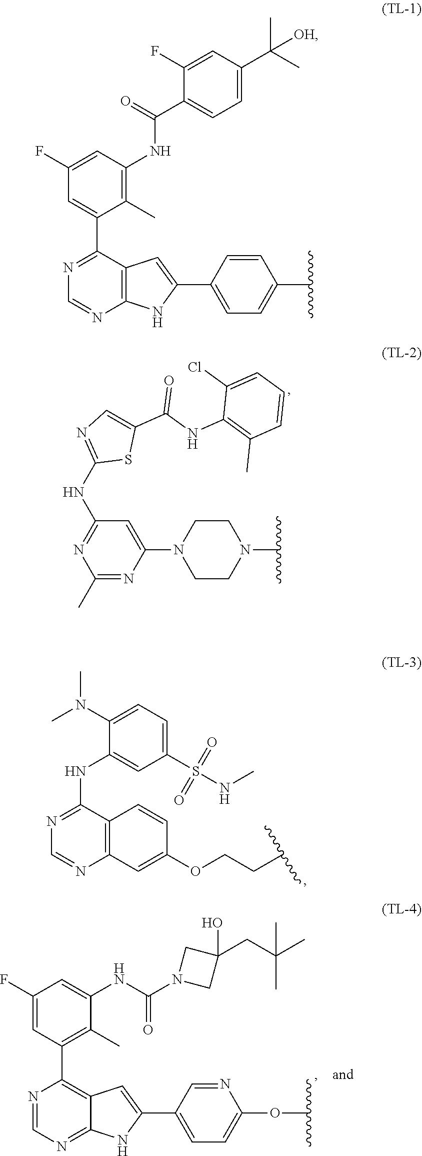 [SMILES: *c1ccc(-c2cc3c(-c4cc(F)cc(NC(=O)c5ccc(C(C)(C)O)cc5F)c4C)ncnc3[nH]2)cc1 ; *N1CCN(c2cc(Nc3ncc(C(=O)Nc4c(C)cccc4Cl)s3)nc(C)n2)CC1 ; *CCOc1ccc2c(Nc3cc(S(=O)(=O)NC)ccc3N(C)C)ncnc2c1 ; *Oc1ccc(-c2cc3c(-c4cc(F)cc(NC(=O)N5CC(O)(CC(C)(C)C)C5)c4C)ncnc3[nH]2)cn1]

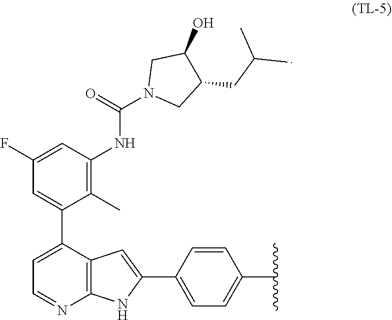 [SMILES: *c1ccc(-c2cc3c(-c4cc(F)cc(NC(=O)N5C[C@@H](CC(C)C)[C@H](O)C5)c4C)ccnc3[nH]2)cc1]

### Targeting Ligase Binder

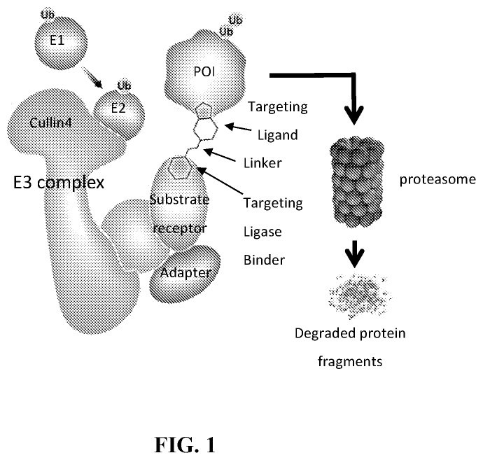

The Targeting Ligase Binder brings a protein of interest (POI) into close proximity to a ubiquitin ligase for tagging with Ubiquitin (Ub), marking the POI for degradation by the ligase through the linking of the Target Ligase Binder bound to the ubiquitin ligase (e.g., an E3 Ubiquitin ligase binding complex), Linker (L), and a Targeting Ligand (TL) bound to the POI. See e.g., FIG. 1.

In an embodiment, the Targeting Ligase Binder has a Formula (TLB-I):

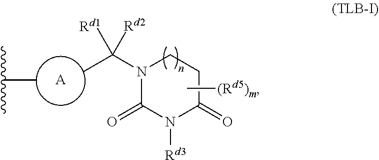

or a pharmaceutically acceptable salt, hydrate, solvate, prodrug, stereoisomer, or tautomer thereof, wherein:
[symbol] denotes the point of attachment to the Linker in Formula (I);
Ring A is a 6-membered aryl, or 5- or 6-membered heteroaryl, each of which is substituted with 0-4 occurrences of R^{d4}.
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d5} attached to the same carbon atom form a C₃₋₄ spirocycloalkyl;
R^{p} is H or C₁₋₆ alkyl;
m is 1 or 2; and
n is 1 or 2.

In an embodiment, n is 1. In an embodiment, R^{d3} is H. In an embodiment, R^{d3} is —CH₂OP(O)(OR^{p})₂. In an embodiment, ring A is selected from the group consisting of phenyl, pyridyl, pyridonyl, pyrazinyl, thiophenyl, pyrazolyl, imidazolyl, and pyrrolyl. In an embodiment, ring A is a 5-membered heteroaryl. In an embodiment, A is a 5-membered nitrogen-containing heteroaryl. In an embodiment, A is a 6-membered heteroaryl. In an embodiment, ring A is a 6-membered nitrogen-containing heteroaryl. In an embodiment, ring A is pyridyl or pyridonyl. In an embodiment, R^{d4} is hydroxyl or C₁₋₆ alkoxyl.

In an embodiment, the Targeting Ligase Binder has a Formula (TLB-II):

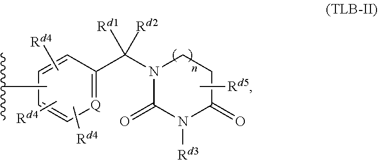

or a pharmaceutically acceptable salt, hydrate, solvate, prodrug, stereoisomer, or tautomer thereof, wherein:
[symbol] denotes the point of attachment to the Linker in Formula (I);
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, R^{d3} is H. In an embodiment, R^{d3} is —CH₂OP(O)(OR^{p})₂.

In an embodiment, R^{d4} is hydroxyl or C₁₋₆ alkoxyl.

In another embodiment, the Targeting Ligase Binder has a Formula (TLB-III):

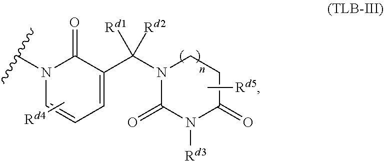

or a pharmaceutically acceptable salt, hydrate, solvate, prodrug, stereoisomer, or tautomer thereof, wherein:
[symbol] denotes the point of attachment to the Linker in Formula (I);
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, R^{d3} is H. In an embodiment, R^{d3} is —CH₂OP(O)(OR^{p})₂. In an embodiment, R^{d1} is H. In an embodiment, R^{d2} is H. In an embodiment, R^{d1} and R^{d2} are both H.

In an embodiment, the Targeting Ligase Binder has a Formula (TLB-IV):

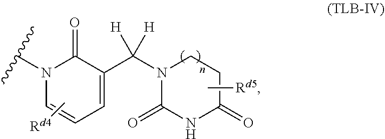

or a pharmaceutically acceptable salt, hydrate, solvate, prodrug, stereoisomer, or tautomer thereof, wherein:
[symbol] denotes the point of attachment to the Linker in Formula (I);
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl; and
n is 1 or 2.

In an embodiment, n is 1. In an embodiment, R^{d3} is H. In an embodiment, R^{d3} is —CH₂OP(O)(OR^{p})₂. In an embodiment, R^{d4} is H or C₁₋₃ alkyl. In an embodiment, R^{d4} is H. In an embodiment, R^{d5} is H or C₁₋₃ alkyl. In an embodiment, R^{d5} is H.

In another embodiment, the Targeting Ligase Binder has a Formula (TLB-V):

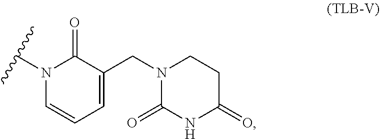 [SMILES: *n1cccc(CN2CCC(=O)NC2=O)c1=O]

or a pharmaceutically acceptable salt, hydrate, solvate, prodrug, stereoisomer, or tautomer thereof.

In an embodiment, the Targeting Ligase Binder has a Formula (TLB-VI):

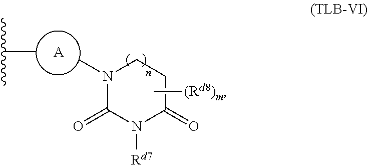

or a pharmaceutically acceptable salt, hydrate, solvate, prodrug, stereoisomer, or tautomer thereof, wherein:
[symbol] denotes the point of attachment to the Linker in Formula (I);
Ring A is a 6-membered aryl or 6-membered heteroaryl, each of which is independently substituted with 0-4 occurrences of R^{d6};
each R^{d6} is independently selected from the group consisting of H, hydroxyl, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{p} is H or C₁₋₆ alkyl;
each R^{d8} is independently selected from the group consisting of H, C₁₋₆ alkyl, halogen, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
m is 1 or 2; and
n is 1 or 2.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a nitrogen-containing 6-membered heteroaryl. In an embodiment, ring A is pyridyl.

In an embodiment, n is 1. In an embodiment, n is 2. In an embodiment, R^{d7} is —CH₂OP(O)(OR^{p})₂. In an embodiment, R^{d7} is H. In an embodiment, R^{d8} is H. In an embodiment, R^{d7} and R^{d8} are both H. In an embodiment, R^{d6} is H. In an embodiment, R^{d6} is selected from the group consisting of H, halogen, C₁₋₆ alkyl, and C₁₋₆ alkoxyl. In an embodiment, R^{d6} is selected from the group consisting of H, halogen, C₁₋₆ alkyl, and C₁₋₆ alkoxyl; and R^{d7}, and R^{d8} are each H.

In an embodiment, the Targeting Ligase Binder has a Formula (TLB-VII):

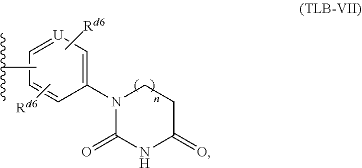

or a pharmaceutically acceptable salt, hydrate, solvate, pro rug, stereoisomer, or tautomer thereof, wherein:
[symbol] denotes the point of attachment to the Linker in Formula (I);
U is —CR^{d6} or N;
each R^{d6} is independently selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

In an embodiment, n is 1. In an embodiment, n is 2. In an embodiment, each R^{d6} is independently selected from the group consisting of H, halogen, C₁₋₃ alkyl, and C₁₋₃ alkoxy. In an embodiment, each R^{d6} is H. In an embodiment, one of R^{d6} is H. In an embodiment, one of R^{d6} is not H.

In an embodiment, the Targeting Ligase Binder has a Formula (TLB-VIII):

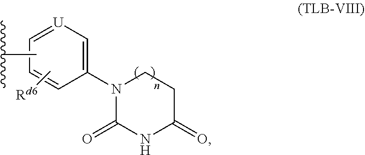

or a pharmaceutically acceptable salt, hydrate, solvate, prodrug, stereoisomer, or tautomer thereof, wherein:
[symbol] denotes the point of attachment to the Linker in Formula (I);
U is —CR^{d6} or N;
R^{d6} is selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

In an embodiment, the Targeting Ligase Binder has a Formula (TLB-IX):

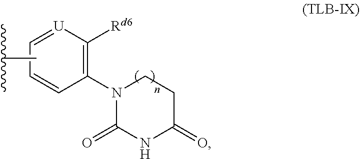

or a pharmaceutically acceptable salt, hydrate, solvate, prodrug, stereoisomer, or tautomer thereof, wherein:
[symbol] denotes the point of attachment to the Linker in Formula (I);
U is independently —CR^{d6} or N;
R^{d6} is selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

In an embodiment, n is 1. In an embodiment, n is 2. In an embodiment, U is N. In an embodiment, U is —CR^{d6}. In an embodiment, each R^{d6} is independently selected from the group consisting of H, methyl, halogen, methoxy, and methoxymethyl. In an embodiment, R^{d6} is H. In an embodiment, R^{d6} is methyl. In an embodiment, R^{d6} is halogen. In an embodiment, R^{d6} is methoxy.

### Linker

In an embodiment, the Linker has Formula (L-I):

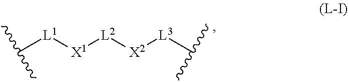

or a pharmaceutically acceptable salt, hydrate, solvate, prodrug, stereoisomer, or tautomer thereof, wherein:
L¹ is selected from the group consisting of a bond, O, NR′, C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand in Formula (I);
X¹ and X² are each independently selected from the group consisting of a bond, carbocyclyl, heterocyclyl, and heteroaryl;
L² is selected from the group consisting of a bond, O, NR′, C(O), C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from a bond, C₁₋₆ alkylene, C₂₋₆ alkenylene, C₂₋₆ alkynylene, C₁₋₆ heteroalkylene, C(O), S(O)₂, O, NR′, *C(O)—C₁₋₉ alkylene, *C(O)—C₁₋₆ alkylene-O, and *C(O)—C₁₋₉ heteroalkylene, wherein * denotes the point of attachment of L³ to X² in (L-I);
   wherein no more than 2 of L¹, X¹, X², L², and L³ can simultaneously be a bond; and
R′ is hydrogen or C₁₋₆ alkyl.

In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl. In an embodiment, one of X¹ and X² is a bond, and the other is a heterocyclyl. In an embodiment, X¹ and X² are each independently selected from piperidinyl and piperazinyl. In an embodiment, X¹ and X² are both piperidinyl. In an embodiment, —X¹-L²-X²— is:

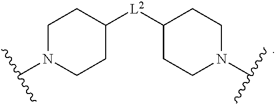

In an embodiment, the Linker is a compound having the following formula:

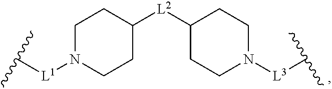

or a pharmaceutically acceptable salt, hydrate, solvate, prodrug, stereoisomer, or tautomer thereof.

In an embodiment, —X¹-L²-X²— forms a spiroheterocyclyl having the structure,

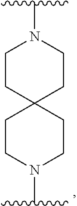 [SMILES: *N1CCC2(CC1)CCN(*)CC2]

substituted with 0-4 occurrences of R^{a}, wherein each R^{a} is independently selected from C₁₋₆ alkyl, C₁₋₆ alkoxyl, and C₁₋₆ hydroxyalkyl.

In an embodiment, —X¹-L²-X²-forms a spiroheterocyclyl having the structure,

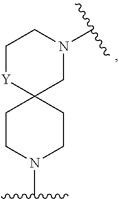

substituted with 0-4 occurrences of R^{b}, wherein Y is selected from CH₂, oxygen, and nitrogen; and each R^{b} is independently selected from C₁₋₆ alkyl, C₁₋₆ alkoxyl, and C₁₋₆ hydroxyalkyl. In an embodiment, X¹ and X² are each a bond.

In an embodiment, L³ is independently selected from the group consisting of —C(O)—, C₂₋₆ alkynylene, or C₁₋₆ heteroalkylene; and L¹ is —C(O)—, C₁₋₈ alkylene, C₁₋₈ heteroalkylene, and *C₁₋₆ alkylene-C(O). In an embodiment, L³ is selected from the group consisting of —C(O)—, —O—C₁₋₆ alkylene, C₂₋₆ alkynylene, and C₁₋₆ heteroalkylene; and L¹ is C₁₋₈ alkylene or C₁₋₈ heteroalkylene. In an embodiment, L³ is —C(O)— or C₁₋₆ heteroalkylene; and L¹ is C₁₋₈ alkylene or C₁₋₈ heteroalkylene. In an embodiment, L³ is a bond or —O—; and L¹ is —C(O)— or C₁₋₈ heteroalkylene. In an embodiment, L³ is selected from the group consisting of —O—, —C(O)—, —S(O)₂—, and C₁₋₆ heteroalkylene; and L¹ is C₁₋₈ alkylene or C₁₋₈ heteroalkylene. In an embodiment, L² is —C(O)—, —NR′—, or C₁₋₆ alkylene.

In an embodiment, L² is —C(O)—, —O—, or C₁₋₆ alkylene. In an embodiment, L² is C₁₋₆ alkylene. In an embodiment, L² is selected from the group consisting of —C(O)—, C₁₋₆ alkylene, C₁₋₆ heteroalkylene, and *C(O)NR′—C₁₋₆ alkylene.

In an embodiment, Y is CH₂, CH(C₁₋₃ alkyl), C(C₁₋₃ alkyl)₂, oxygen, NH, or N(C₁₋₃ alkyl).

### Targeting Ligand-Linkers

In an embodiment, the Targeting Ligase Binder-Linker has Formula (TLB-L-I):

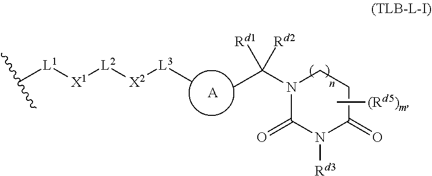

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′—, —C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O), —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-I);
wherein no more than 2 of L¹, X¹, X², L², and L³ can simultaneously be a bond;
Ring A is a 6-membered aryl, or 5- or 6-membered heteroaryl, each of which is substituted with 0-4 occurrences of R^{d4};
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d5} attached to the same carbon atom form a C₃₋₄ spirocycloalkyl;
R^{p} is H or C₁₋₆ alkyl;
m is 1 or 2; and
n is 1 or 2.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a 5-membered heteroaryl. In an embodiment, ring A is a 5-membered nitrogen-containing heteroaryl. In an embodiment, ring A is a 6-membered heteroaryl. In an embodiment, ring A is a 6-membered nitrogen-containing heteroaryl. In an embodiment, ring A is pyridyl. In an embodiment, n is 1.

In an embodiment, R^{d3} is H. In an embodiment, R^{d3} is —CH₂OP(O)(OR^{p})₂.

In an embodiment, the Targeting Ligase Binder-Linker has Formula (TLB-L-II):

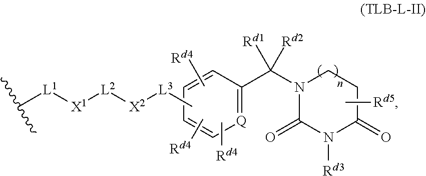

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′—, —C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O), —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-II);
wherein no more than 2 of L¹, X¹, X², L², and L³ can simultaneously be a bond;
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, R^{d3} is H. In an embodiment, R^{d3} is —CH₂OP(O)(OR^{P})₂.

In an embodiment, the Targeting Ligase Binder-Linker has Formula (TLB-L-III):

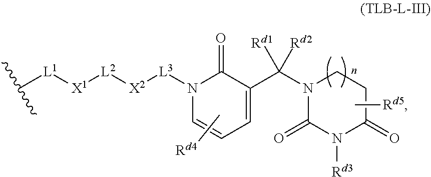

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-III);
   wherein no more than 2 of L¹, X¹, X², L², and L³ can simultaneously be a bond;
R′ is hydrogen or C₁₋₆ alkyl;
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, R^{d3} is H. In an embodiment, R^{d3} is —CH₂OP(O)(OR^{p})₂.

In an embodiment, the Targeting Ligase Binder-Linker has Formula (TLB-L-IV):

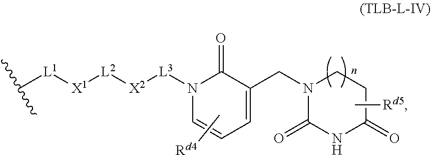

or a pharmaceutically acceptable salt, hydrate, solvate, prodrug, stereoisomer, or tautomer thereof, wherein
[symbol] denotes the point of attachment to the Targeting Ligand in Formula (I);
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-IV);
wherein no more than 2 of L¹, X¹, X², L², and L³ can simultaneously be a bond;
R′ is hydrogen or C₁₋₆ alkyl;
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
n is 1 or 2.

In an embodiment, n is 1. In an embodiment, n is 2.

In an embodiment, the Targeting Ligase Binder-Linker has Formula (TLB-L-V):

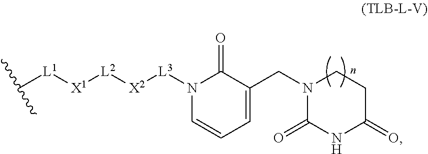

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-V);
wherein no more than 2 of L¹, X¹, X², L², and L³ can simultaneously be a bond;
R′ is hydrogen or C₁₋₆ alkyl; and
n is 1 or 2.

In an embodiment, n is 1. In an embodiment, n is 2. In an embodiment, L³ is selected from the group consisting of —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl. In an embodiment, one of X¹ and X² is a bond, and the other is a heterocyclyl.

In an embodiment, the Targeting Ligase Binder-Linker, or a pharmaceutically acceptable salt, hydrate, solvate, prodrug, stereoisomer, or tautomer thereof, has a Formula selected from:

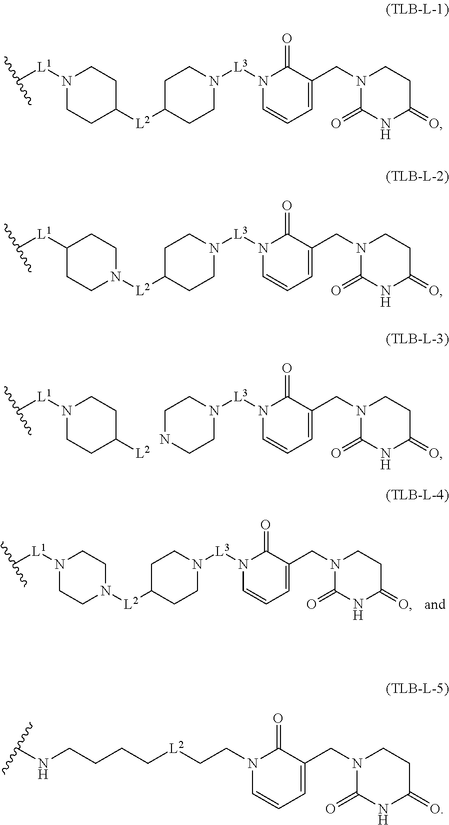

In an embodiment, the Targeting Ligase Binder-Linker has Formula (TLB-L-VI):

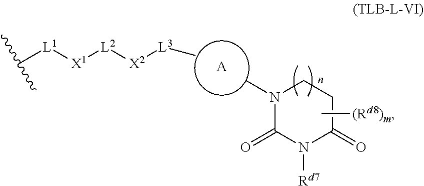

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-L-VI);
wherein no more than 2 of L¹, X¹, X², L², and L³ can simultaneously be a bond;
R′ is hydrogen or C₁₋₆ alkyl;
Ring A is a 6-membered aryl or 6-membered heteroaryl, each of which is independently substituted with 0-4 occurrences of R^{d6};
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{p} is H or C₁₋₆ alkyl;
each R^{d8} is independently selected from the group consisting of H, C₁₋₆ alkyl, halogen, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
m is 1 or 2; and
n is 1 or 2.

In an embodiment, n is 1. In an embodiment, n is 2. In an embodiment, R^{d3} is —CH₂OP(O)(OR^{p})₂. In an embodiment, R^{d3} is H.

In an embodiment, the Targeting Ligase Binder-Linker has Formula (TLB-L-VII):

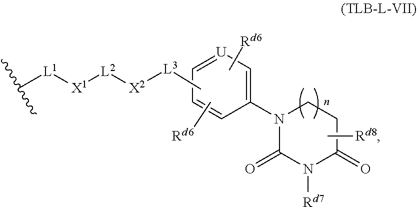

or a pharmaceutically acceptable salt, hydrate, solvate, prodrug, stereoisomer, or tautomer thereof, wherein:
[symbol] denotes the point of attachment to the Targeting Ligand in Formula (I);
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (TLB-VII;
wherein no more than 2 of L¹, X¹, X², L², and L³ can simultaneously be a bond;
R′ is hydrogen or C₁₋₆ alkyl;
U is —CR^{d6} or N;
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d8} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, n is 1. In an embodiment, n is 2. In an embodiment, R^{d3} is —CH₂OP(O)(OR^{p})₂. In an embodiment, R^{d3} is H. In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, one of X¹ and X² is not a bond. In an embodiment, one of X¹ and X² is a bond, and the other is a carbocyclyl or heterocyclyl. In an embodiment, one of X¹ and X² is a bond, and the other is a heterocyclyl.

In an embodiment, the Targeting Ligase Binder-Linker has Formula (TLB-L-VIII or TLB-L-IX):

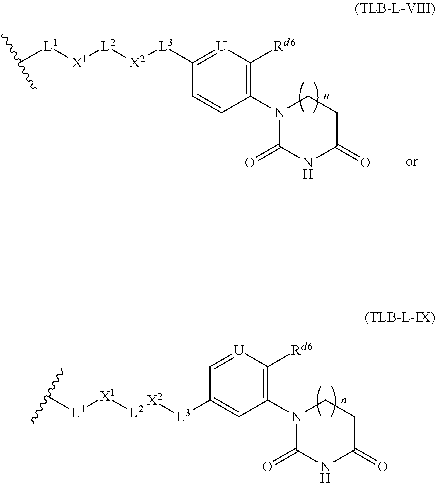

or a pharmaceutically acceptable salt, hydrate, solvate, prodrug, stereoisomer, or tautomer thereof, wherein the point of attachment to the Targeting Ligand is through L¹.

In an embodiment, n is 1. In an embodiment, n is 2.

In an embodiment, the Targeting Ligase Binder-Linker, or a pharmaceutically acceptable salt, hydrate, solvate, prodrug, stereoisomer, or tautomer thereof, has a Formula selected from:

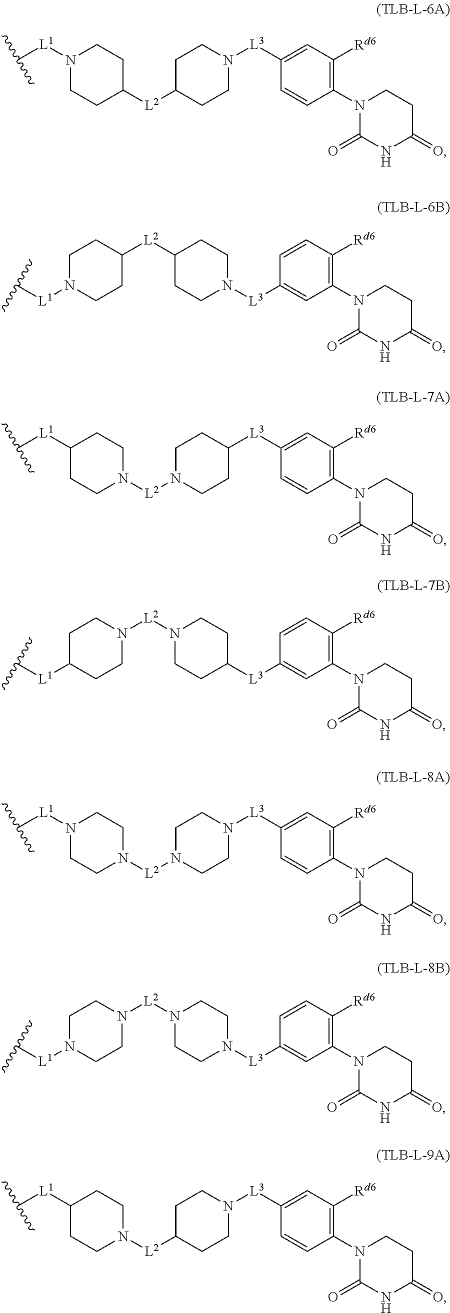

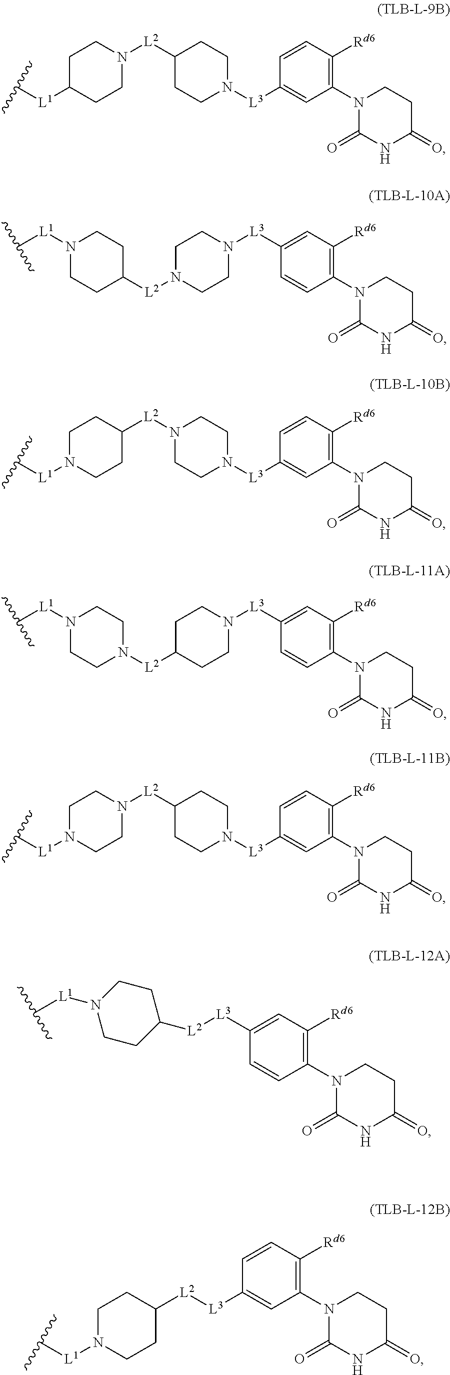

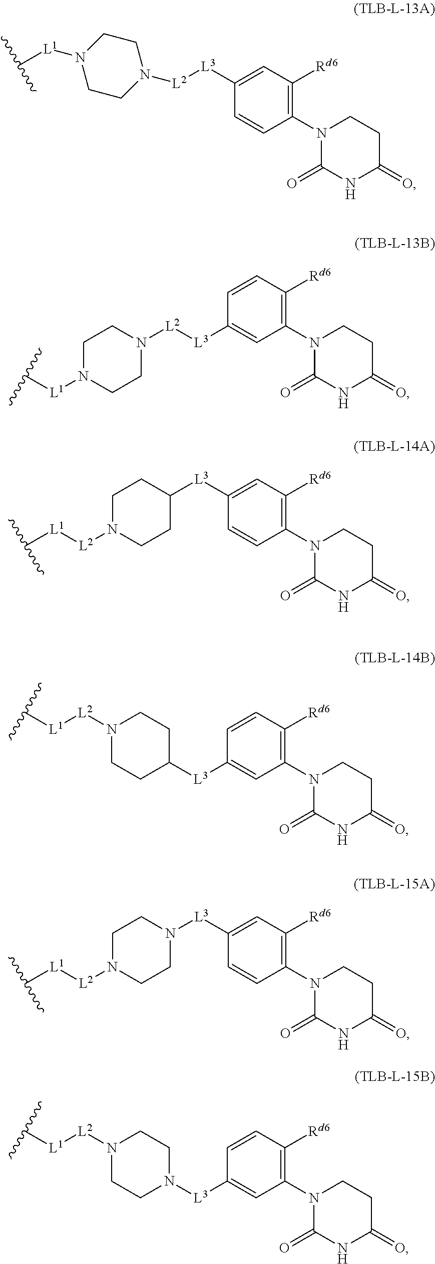

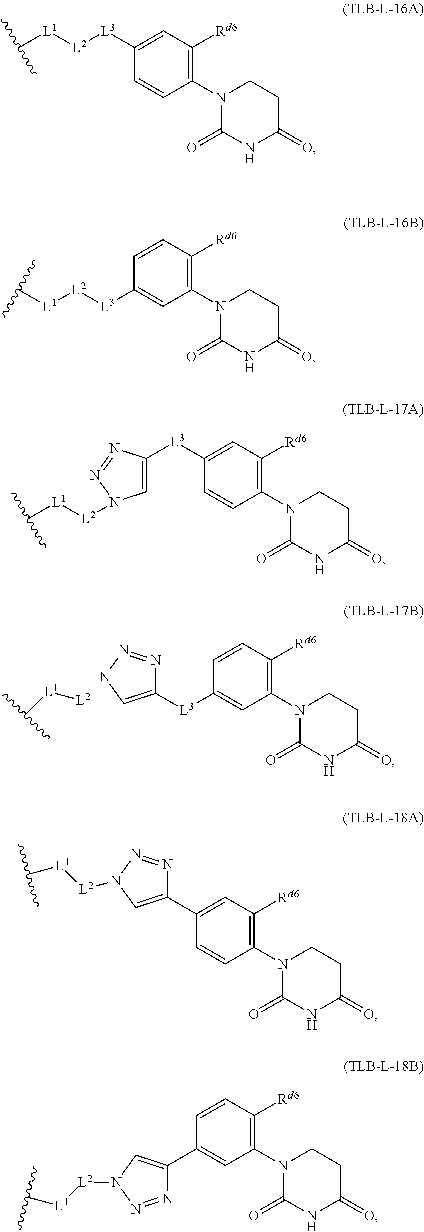

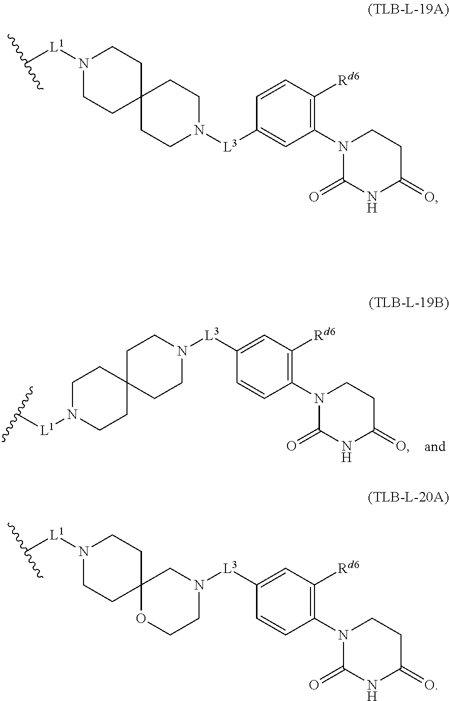

### Compound Formulas

In another aspect, the disclosure provides a compound of Formula (BF-I):

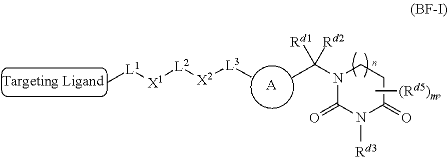

or a pharmaceutically acceptable salt, hydrate, solvate, prodrug, stereoisomer, or tautomer thereof, wherein:
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene;
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-I);
wherein no more than 2 of L¹, X¹, X², L², and L³ can simultaneously be a bond;
Ring A is a 6-membered aryl, or 5- or 6-membered heteroaryl, each of which is substituted with 0-4 occurrences of R^{d4};
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d5} attached to the same carbon atom form a C₃₋₄ spirocycloalkyl;
R^{p} is H or C₁₋₆ alkyl;
m is 1 or 2; and
n is 1 or 2, wherein the Targeting Ligand is a group capable of binding to a Target Protein.

In an embodiment, ring A is selected from the group consisting of phenyl, pyridyl, pyridonyl, pyrazinyl, thiophenyl, pyrazolyl, imidazolyl, and pyrrolyl. In an embodiment, ring A is a 5-membered heteroaryl. In an embodiment, ring A is a 5-membered nitrogen-containing heteroaryl. In an embodiment, ring A is a 6-membered heteroaryl. In an embodiment, ring A is a 6-membered nitrogen-containing heteroaryl. In an embodiment, ring A is pyridyl. In an embodiment, n is 1. In an embodiment, n is 2. In an embodiment, R^{d3} is —CH₂OP(O)(OR^{p})₂. In an embodiment, n R^{d3} is H.

In another aspect, the disclosure provides a compound of Formula (BF-II):

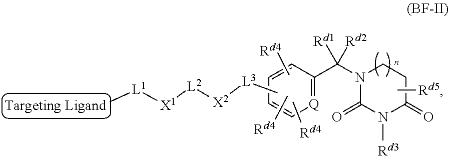

or a pharmaceutically acceptable salt, hydrate, solvate, prodrug, stereoisomer, or tautomer thereof, wherein:
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-II);
wherein no more than 2 of L¹, X¹, X², L², and L³ can simultaneously be a bond;
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2, wherein the Targeting Ligand is a group capable of binding to a Target Protein.

In an embodiment, n is 1. In another aspect, n is 2. In an embodiment, R^{d3} is —CH₂OP(O)(OR^{p})₂. In an embodiment, R^{d3} is H.

In another aspect, the disclosure provides a compound of Formula (BF-III):

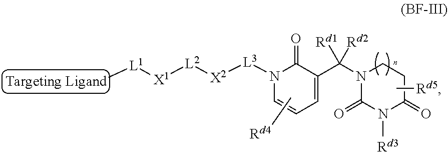

or a pharmaceutically acceptable salt, hydrate, solvate, prodrug, stereoisomer, or tautomer thereof, wherein:
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-III);
wherein no more than 2 of L¹, X¹, X², L², and L³ can simultaneously be a bond;
R′ is hydrogen or C₁₋₆ alkyl;
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2, wherein the Targeting Ligand is a group capable of binding to a Target Protein.

In an embodiment, n is 1. In an embodiment, n is 2. In an embodiment, R^{d3} is —CH₂OP(O)(OR^{p})₂. In an embodiment, R^{d3} is H. In an embodiment, —X¹-L²-X²— is:

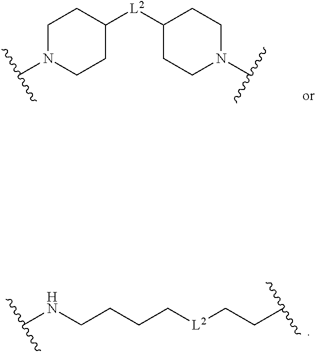

In an embodiment, L¹ is —O— or C₁₋₆ alkylene. In an embodiment, R^{d1} and R^{d2} are both methyl. In an embodiment, R^{d1} and R^{d2} are both H. In another aspect, R^{d4} is H or C₁₋₃ alkyl. In an embodiment, R^{d5} is H or C₁₋₃ alkyl.

In another aspect, the disclosure provides a compound of Formula (BF-IV):

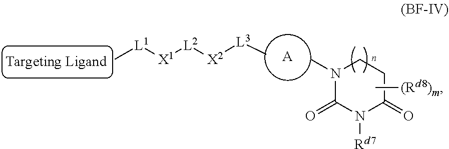

or a pharmaceutically acceptable salt, hydrate, solvate, prodrug, stereoisomer, or tautomer thereof, wherein:
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-IV);
wherein no more than 2 of L¹, X¹, X², L², and L³ can simultaneously be a bond;
R′ is hydrogen or C₁₋₆ alkyl;
Ring A is a 6-membered aryl or 6-membered heteroaryl, each of which is independently substituted with 0-4 occurrences of R^{d6};
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{p} is H or C₁₋₆ alkyl;
each R^{d8} is independently selected from the group consisting of H, C₁₋₆ alkyl, halogen, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
m is 1 or 2; and
n is 1 or 2, wherein the Targeting Ligand is a group capable of binding to a Target Protein.

In an embodiment, the compound has the Formula (BF-V-A) or (BF-V-B):

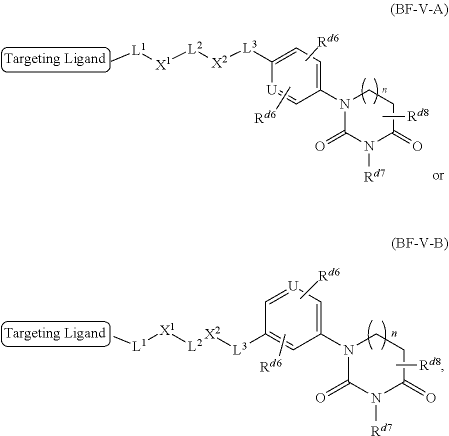

or a pharmaceutically acceptable salt, hydrate, solvate, prodrug, stereoisomer, or tautomer thereof, wherein:
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-V-A or BF-V-B);
wherein no more than 2 of L¹, X¹, X², L², and L³ can simultaneously be a bond;
R′ is hydrogen or C₁₋₆ alkyl;
U is —CR^{d6} or N;
each R^{d6} is independently selected from the group consisting of H, oxo, C₁₋₆ alkyl, halogen, C₁₋₆ alkoxyl, C₁₋₆ alkoxyalkyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d8} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2, wherein the Targeting Ligand is a group capable of binding to a Target Protein.

In an embodiment, n is 1. In another aspect, n is 2. In another aspect, R^{d7} is —CH₂OP(O)(OR^{p})₂. In another aspect, R^{d7} is H. In another aspect, U is —CR^{d6}. In another aspect, R^{d8} is H. In another aspect, R^{d7} and R^{d8} are each independently H. In another aspect, R^{d6} is H. In another aspect, R^{d6} is selected from the group consisting of H, halogen, C₁₋₆ alkyl, and C₁₋₆ alkoxyl. In another aspect, R^{d6} is selected from the group consisting of H, halogen, C₁₋₆ alkyl, and C₁₋₆ alkoxyl; and R^{d7}, and R^{d8} are each H.

In an embodiment, L¹-X¹-L²-X²-L³ is selected from the group consisting of:

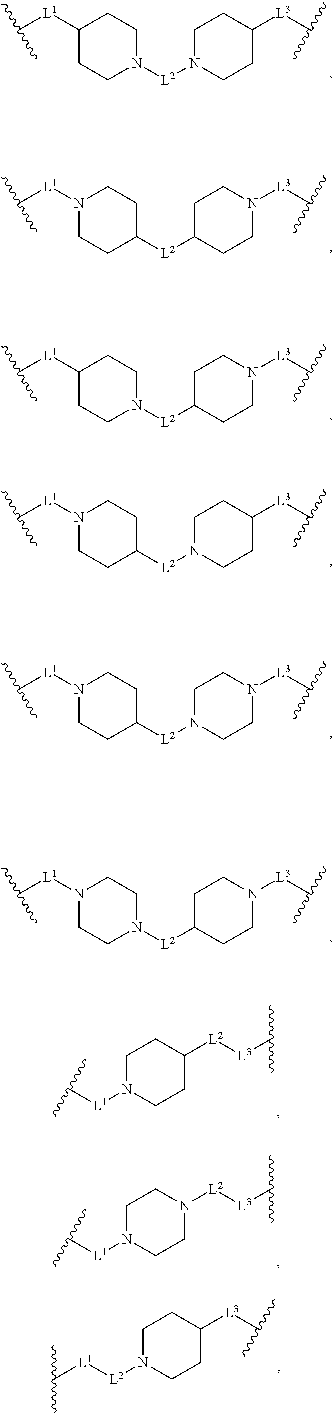

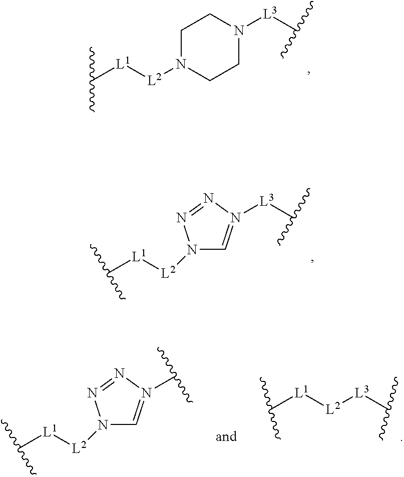

In an embodiment, L³ is selected from the group consisting of a bond, —O—, —C(O)—, —S(O)₂—, C₁₋₆ alkylene, C₂₋₆ alkynylene, and C₁₋₆ heteroalkylene.

### Intermediates

Another embodiment is a compound of Formula (ILB-I):

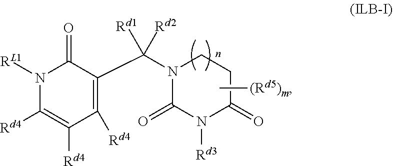

or a pharmaceutically acceptable salt, hydrate, solvate, prodrug, stereoisomer, or tautomer thereof, wherein:
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, C₁₋₆ alkyl, halogen, C₁₋₆ haloalkyl, C₁₋₆ alkoxyl; C₁₋₆ alkoxyalkyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d5} attached to the same carbon atom form a C₃₋₄ spirocycloalkyl;
R^{L1} is selected from the group consisting of C₁₋₆ alkyl, C₂₋₆ alkenyl, C₂₋₆ alkynyl, C₂₋₆ hydroxyalkyl, —(CH₂)₂₋₆NHR^{c}, C₃₋₆ heteroalkyl, C₂₋₆ haloalkyl, —(CH₂)₁₋₃C(O)OH, —(CH₂)₁₋₃C(O)H, —(CH₂)₁₋₃O(CH₂)₁₋₃C(O)H, —(CH₂)₀₋₃C₃₋₇ carbocyclyl, —(CH₂)₀₋₃ heterocyclyl, C₆ aryl, and heteroaryl, wherein the carbocyclyl, heterocyclyl, aryl, and heteroaryl is substituted with 0-2 occurrences of —O-heterocyclyl, —O-carbocyclyl, —C(O)— heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, or C₁₋₆ heteroalkyl;
R^{p} is H or C₁₋₆ alkyl;
m is 1 or 2; and
n is 1 or 2.

In an embodiment, n is 1. In an embodiment, n is 2. In an embodiment, R^{d3} is —CH₂OP(O)(OR^{p})₂. In an embodiment, R^{d3} is H. In an embodiment, R^{d4} is H.

In an embodiment, R^{L1} is selected from the group consisting of C₂₋₆ alkenyl, C₂₋₆ hydroxyalkyl, —(CH₂)₁₋₃C(O)OH, —(CH₂)₁₋₃C(O)H, —(CH₂)₁₋₃O(CH₂)₁₋₃C(O)H, —(CH₂)₀₋₃ heterocyclyl, wherein the heterocyclyl, is substituted with 0-2 occurrences of —O-heterocyclyl.

Another embodiment is a compound or a pharmaceutically acceptable salt, hydrate, solvate, prodrug, stereoisomer, or tautomer thereof, selected from:

selected from:

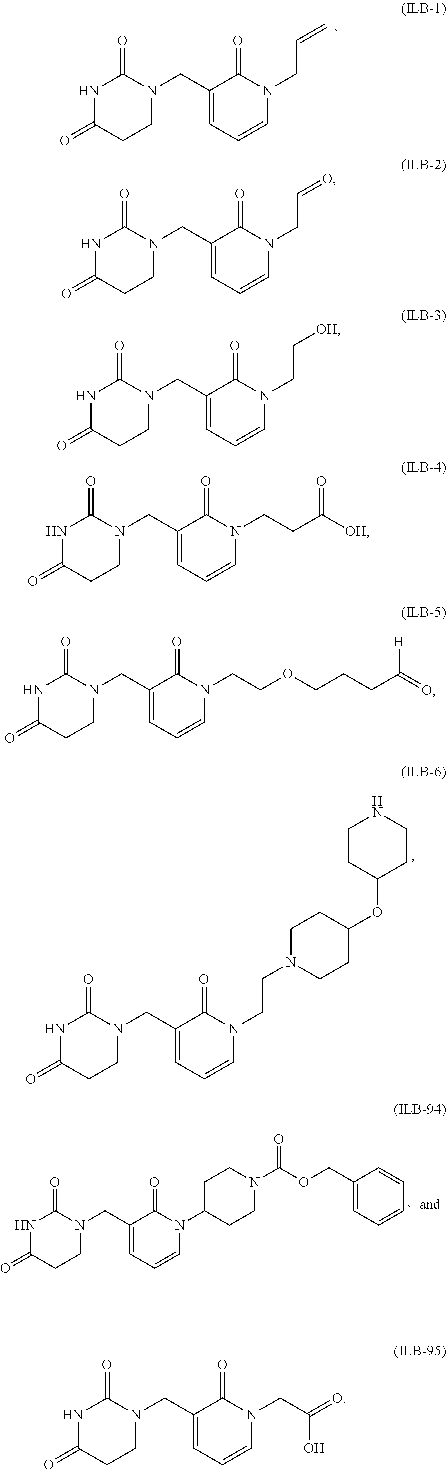

Another embodiment is a compound of Formula (ILB-II):

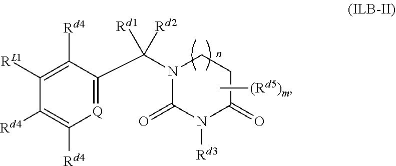

or a pharmaceutically acceptable salt, hydrate, solvate, prodrug, stereoisomer, or tautomer thereof, wherein:
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂(O)(CH₂)₂Si(CH₃)₃, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, C₁₋₆ alkyl, halogen, C₁₋₆ haloalkyl, C₁₋₆ alkoxyl; C₁₋₆ alkoxyalkyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
   or two R^{d5} together with the carbon atoms to which they are attached form a cycloalkyl;
   or two R^{d5} attached to the same carbon atom form a C₃₋₄ spirocycloalkyl;
R^{L1} is selected from the group consisting of C₂₋₆ alkenyl, C₂₋₆ alkynyl, C₂₋₆ hydroxyalkyl, —(CH₂)₂₋₆NHR^{c}, C₃₋₆ heteroalkyl, C₂₋₆ haloalkyl, —(CH₂)₀₋₃C(O)OH, —(CH₂)₀₋₃C(O)H, —(CH₂)₀₋₃C₃₋₇ carbocyclyl, —(CH₂)₀₋₃ heterocyclyl, C₆ aryl, and heteroaryl, wherein the carbocyclyl, heterocyclyl, aryl, and heteroaryl is substituted with 0-2 occurrences of —O-heterocyclyl, —O-carbocyclyl, —C(O)-heterocyclyl, —C(O)— carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, or C₁₋₆ heteroalkyl;
R^{p} is H or C₁₋₆ alkyl;
m is 1 or 2; and
n is 1 or 2.

In an embodiment, n is 1. In an embodiment, n is 2. In an embodiment, R^{d3} is —CH₂OP(O)(OR^{p})₂. In an embodiment, R^{d3} is H.

In an embodiment, Q is N; and R^{L1} is —(CH₂)₀₋₃C(O)OH.

In an embodiment, Q is CR^{d4}; and R^{L1} is C₂₋₆ hydroxyalkyl, —(CH₂)₀₋₃C(O)OH, and —(CH₂)₀₋₃C(O)H.

Another embodiment is a compound or a pharmaceutically acceptable salt, hydrate, solvate, prodrug, stereoisomer, or tautomer thereof, selected from:

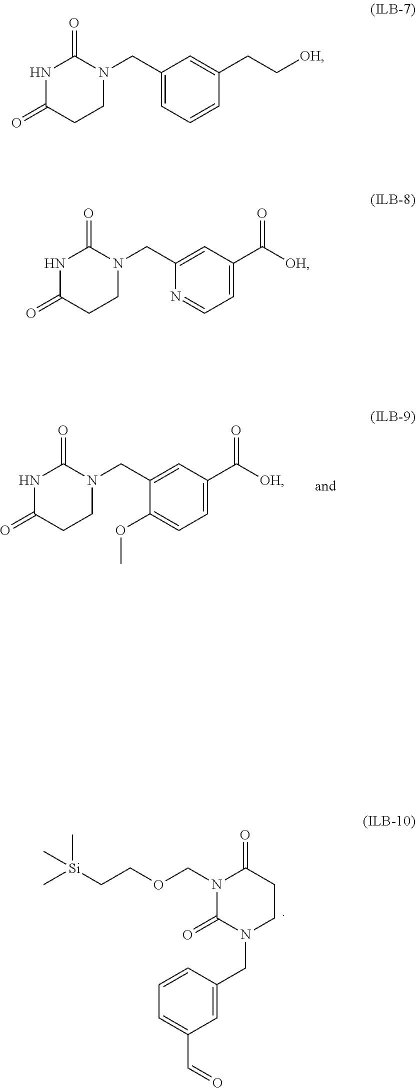 [SMILES: O=C1CCN(Cc2cccc(CCO)c2)C(=O)N1 ; O=C1CCN(Cc2cc(C(=O)O)ccn2)C(=O)N1 ; COc1ccc(C(=O)O)cc1CN1CCC(=O)NC1=O ; C[Si](C)(C)CCOCN1C(=O)CCN(Cc2cccc(C=O)c2)C1=O]

Another embodiment is a compound of Formula (ILB-III):

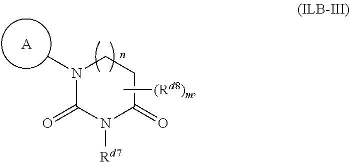

or a pharmaceutically acceptable salt, hydrate, solvate, prodrug, stereoisomer, or tautomer thereof, wherein:

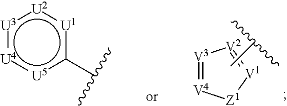

[symbol] denotes the point of attachment to the base molecule of (ILB-III);
U¹, U², U³, U⁴, and U⁵ are each independently N or CR^{d6} or CR^{L2}, wherein no more than three of U¹, U², U³, U⁴, and U⁵ can be N, and wherein one of U¹, U², U³, U⁴, and U⁵ is CR^{L2} and the remaining are CR^{d6};
Z¹ is selected from the group consisting of O, S, NR^{d6a}; or NR^{L2a}
V¹, V², V³, and V⁴ are each independently N or C, wherein no more than two of V¹, V², V³, and V⁴ can be N, and wherein one of Z¹, V¹, V², V³, and V⁴ is substituted with R^{L2}, one of V¹, V², V³, and V⁴ is the point of attachment to the base molecule of (ILB-III), and the remaining are substituted with R^{d6};
each R^{d6} is independently selected from the group consisting of H, hydroxyl, oxo, polyethylene glycol (PEG), halogen, C₁₋₃ alkyl, C₁₋₃ alkoxyl, C₁₋₆ haloalkyl, C₁₋₆ heteroalkyl, and —OC₁₋₇ heteroalkyl;
each R^{d6a} is independently selected from the group consisting of H, polyethylene glycol (PEG), C₁₋₃ alkyl, C₃₋₆ cycloalkyl, C₂₋₆ haloalkyl, and C₂₋₆ heteroalkyl;
R^{d7} is H, —CH₂OC(O)R^{p}, —CH₂OP(O)OHOR^{p}, —CH₂OP(O)(R^{p})₂, and —CH₂OP(O)(OR^{p})₂;
each R^{d8} is independently selected from the group consisting of H, C₁₋₆ alkyl, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
R^{L2} is selected from the group consisting of H, hydroxyl, C₂₋₆ alkenyl, C₂₋₆ alkynyl, C₂₋₆ hydroxyalkyl, —(CH₂)₂₋₆NHR^{c}, —(CH₂)₂₋₆NR^{c}R^{d}, —O—(CH₂)₂₋₆NHR^{c}, C₁₋₈ heteroalkyl, C₁₋₆ haloalkyl, —SO₂—NH—(CH₂)₂₋₆NHR^{c}, —(CH₂)₀₋₃C(O)OH, —(CH₂)₀₋₃C(O)OR^{c}, —O—C₂₋₆ alkenyl, —O—(CH₂)₀₋₃C(O)H, —(CH₂)₁₋₃C(O)H, —O—(CH₂)₁₋₃C(O)OH, —(CH₂)₀₋₃ heterocyclyl, —C(O)—(CH₂)₀₋₃ heterocyclyl, —O—(CH₂)₀₋₃ heterocyclyl, —O—(CH₂)₀₋₃C(O)-heterocyclyl, —C₂₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, —(CH₂)₂₋₆N(R^{c})₂, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{L2a} is selected from the group consisting of H, C₃₋₆ alkenyl, C₃₋₆ alkynyl, C₂₋₆ hydroxyalkyl, —(CH₂)₂₋₆NHR^{c}, —(CH₂)₂₋₆NR^{c}R^{d}, C₂₋₆ heteroalkyl, C₂₋₆ haloalkyl, —(CH₂)₁₋₃C(O)OH, —(CH₂)₁₋₃C(O)H, —(CH₂)₀₋₃ heterocyclyl, —C(O)—(CH₂)₀₋₃ heterocyclyl, —C₃₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, C₁₋₆ heteroalkyl, and —C(O)OC₁₋₆ alkyl;
R^{d} is H or C₁₋₄ alkyl; or R^{c} and R^{d} together with the nitrogen atom to which they are attached form a heterocyclyl substituted with 0-2 occurrences of —O-heterocyclyl,
R^{p} is H or C₁₋₆ alkyl;
m is 1 or 2; and
n is 1 or 2.

Another embodiment is a compound of Formula (ILB-III):

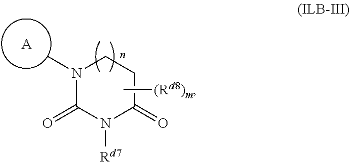

or a pharmaceutically acceptable salt, hydrate, solvate, prodrug, stereoisomer, or tautomer thereof, wherein:
Ring A is selected from the group consisting of:

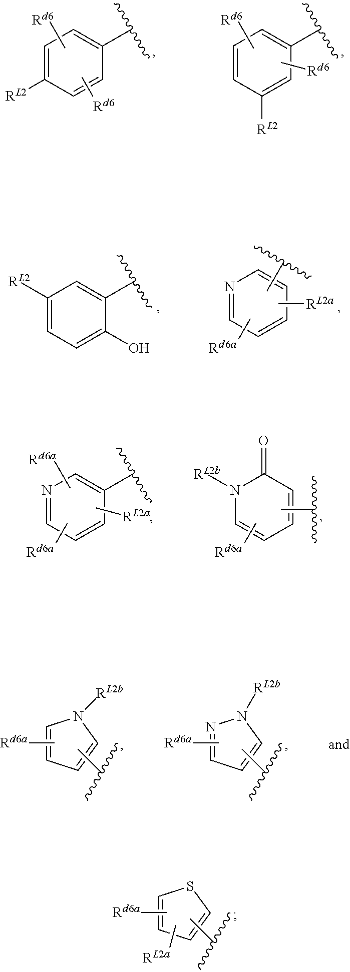

[symbol] denotes the point of attachment to the base molecule of (ILB-III);
each R^{d6} is independently selected from the group consisting of H, oxo, polyethylene glycol (PEG), halogen, C₁₋₃ alkyl, C₁₋₃ alkoxyl, C₁₋₆ haloalkyl, C₁₋₆ heteroalkyl, and —OC₁₋₇ heteroalkyl;
each R^{d6a} is independently selected from the group consisting of H, hydroxyl, oxo, polyethylene glycol (PEG), halogen, C₁₋₃ alkyl, C₁₋₃ alkoxyl, C₁₋₆ haloalkyl, C₁₋₆ heteroalkyl, and —OC₁₋₇ heteroalkyl;
R^{d7} is H, —CH₂OC(O)R^{p}, —CH₂OP(O)OHOR^{p}, —CH₂OP(O)(R^{p})₂, and —CH₂OP(O)(OR^{p})₂;
each R^{d8} is independently selected from the group consisting of H, C₁₋₆ alkyl, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
R^{L2} is selected from the group consisting of hydroxyl, C₂₋₆ alkenyl, C₂₋₆ alkynyl, C₂₋₆ hydroxyalkyl, —(CH₂)₂₋₆NHR^{c}, —(CH₂)₂₋₆NR^{c}R^{d}, —O—(CH₂)₂₋₆NHR^{c}, C₄₋₈ heteroalkyl, C₂₋₆ haloalkyl, —SO₂—NH—(CH₂)₂₋₆NHR^{c}, —(CH₂)₀₋₃C(O)OH, —(CH₂)₀₋₃C(O)OR^{c}, —O—C₂₋₆ alkenyl, —O—(CH₂)₀₋₃C(O)H, —(CH₂)₀₋₃C(O)H, —O—(CH₂)₁₋₃C(O)OH, —(CH₂)₀₋₃ heterocyclyl, —C(O)—(CH₂)₀₋₃ heterocyclyl, —O—(CH₂)₀₋₃ heterocyclyl, —O—(CH₂)₀₋₃C(O)-heterocyclyl, —C₂₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, —(CH₂)₂₋₆N(R^{c})₂, heterocyclyl, heteroaryl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl, wherein the heterocyclyl and heteroaryl is substituted with 0-2 occurrences of halogen;
R^{L2a} is selected from the group consisting of H, hydroxyl, C₂₋₆ alkenyl, C₂₋₆ alkynyl, C₂₋₆ hydroxyalkyl, —(CH₂)₂₋₆NHR^{c}, —(CH₂)₂₋₆NR^{c}R^{d}, —O—(CH₂)₂₋₆NHR^{c}, C₁₋₈ heteroalkyl, C₁₋₆ haloalkyl, —SO₂—NH—(CH₂)₂₋₆NHR^{c}, —(CH₂)₀₋₃C(O)OH, —(CH₂)₀₋₃C(O)OR^{c}, —O—C₂₋₆ alkenyl, —O—(CH₂)₀₋₃C(O)H, —(CH₂)₀₋₃C(O)H, —O—(CH₂)₁₋₃C(O)OH, —(CH₂)₀₋₃ heterocyclyl, —C(O)—(CH₂)₀₋₃ heterocyclyl, —O—(CH₂)₀₋₃ heterocyclyl, —O—(CH₂)₀₋₃C(O)-heterocyclyl, —C₂₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, —(CH₂)₂₋₆N(R^{c})₂, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl, wherein the heterocyclyl is substituted with 0-2 occurrences of halogen;
R^{L2b} is selected from the group consisting of H, polyethylene glycol (PEG), C₁₋₃ alkyl, C₃₋₆ cycloalkyl, C₃₋₆ alkenyl, C₃₋₆ alkynyl, C₂₋₆ hydroxyalkyl, —(CH₂)₂₋₆NHR^{c}, —(CH₂)₂₋₆NR^{c}R^{d}, C₂₋₆ heteroalkyl, C₂₋₆ haloalkyl, —(CH₂)₁₋₃C(O)OH, —(CH₂)₁₋₃C(O)H, —(CH₂)₀₋₃ heterocyclyl, —C(O)—(CH₂)₀₋₃ heterocyclyl, —C₃₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, C₁₋₆ heteroalkyl, and —C(O)OC₁₋₆ alkyl;
R^{d} is H or C₁₋₄ alkyl; or R^{c} and R^{d} together with the nitrogen atom to which they are attached form a heterocyclyl substituted with 0-2 occurrences of —O-heterocyclyl,
R^{p} is H or C₁₋₆ alkyl;
m is 1 or 2; and
n is 1 or 2.

In an embodiment, ring A is selected from the group consisting of:

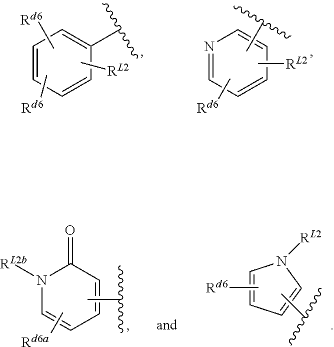

In an embodiment, n is 1. In an embodiment, n is 2. In an embodiment, R^{d7} is —CH₂OP(O)(OR^{p})₂. In an embodiment, R^{d7} is H.

In an embodiment, each R^{d6} is independently selected from the group consisting of H, polyethylene glycol (PEG), halogen, C₁₋₃ alkyl, and C₁₋₃ alkoxyl.

In an embodiment each R^{d6a} is independently halogen.

In an embodiment, R^{L2} is selected from the group consisting of hydroxyl, C₂₋₆ alkynyl, —O—(CH₂)₂₋₆NHR^{c}, C₄₋₈ heteroalkyl, —SO₂—NH—(CH₂)₂₋₆NHR^{c}, —O—C₂₋₆ alkenyl, —(CH₂)₀₋₃C(O)H, —O—(CH₂)₁₋₃C(O)OH, —(CH₂)₀₋₃ heterocyclyl, —C(O)—(CH₂)₀₋₃ heterocyclyl, —O—(CH₂)₀₋₃ heterocyclyl, —O—(CH₂)₀₋₃C(O)-heterocyclyl, —C₂₋₆ alkynyl-heterocyclyl, and heteroaryl, wherein the alkynyl, heterocyclyl, heteroalkyl, and heteroaryl is substituted with 0-2 occurrences of halogen, hydroxyl, —(CH₂)₂₋₆NHR^{c}, heterocyclyl, heteroaryl, —C(O)-heterocyclyl, wherein the heterocyclyl and heteroaryl is substituted with 0-2 occurrences of halogen.

In an embodiment, the heterocyclyl is selected from the group consisting of:

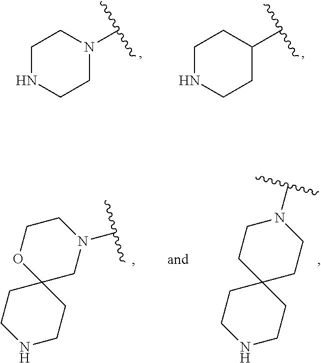

wherein [symbol] denotes the point of attachment to the base molecule of (ILB-III).

In an embodiment, R^{L2a} is H.

In an embodiment, R^{c} is H or —C(O)OC₁₋₆ alkyl.

In an embodiment, R^{d} is H or C₁₋₄ alkyl.

Another embodiment is a compound or a pharmaceutically acceptable salt, hydrate, solvate, prodrug, stereoisomer, or tautomer thereof, selected from:

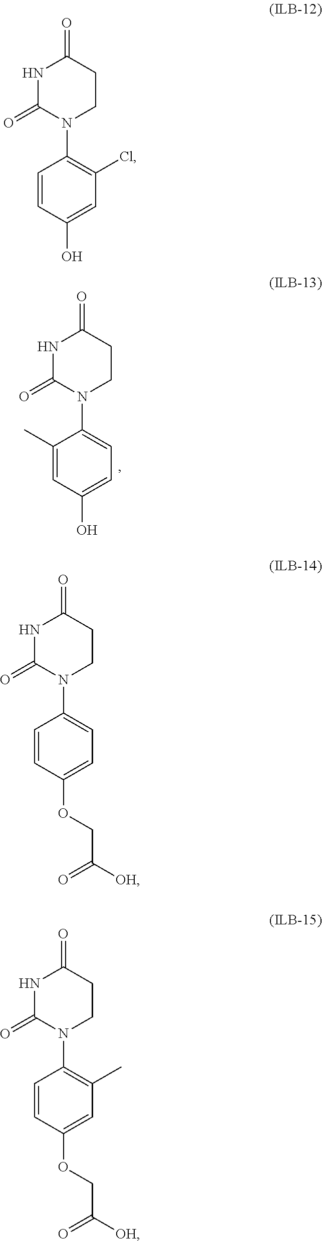

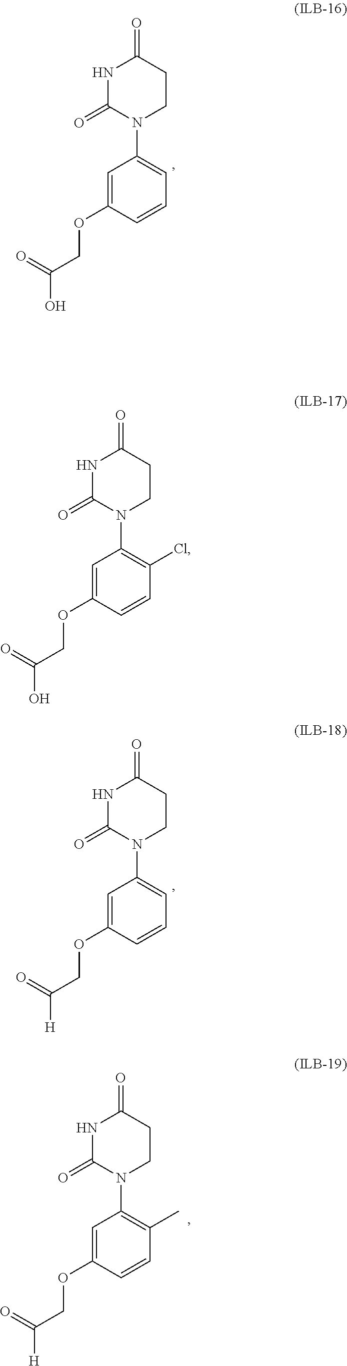 [SMILES: O=C(O)COc1cccc(N2CCC(=O)NC2=O)c1 ; O=C(O)COc1ccc(Cl)c(N2CCC(=O)NC2=O)c1 ; O=CCOc1cccc(N2CCC(=O)NC2=O)c1 ; Cc1ccc(OCC=O)cc1N1CCC(=O)NC1=O]

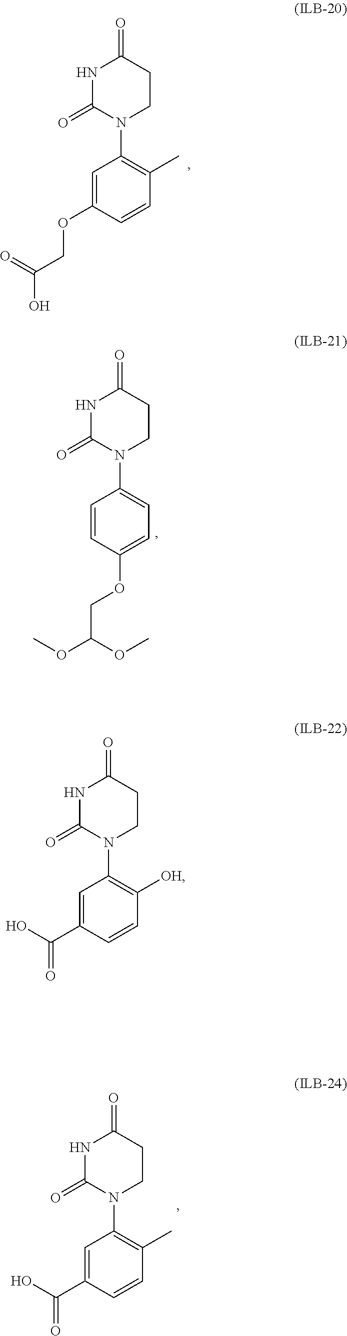

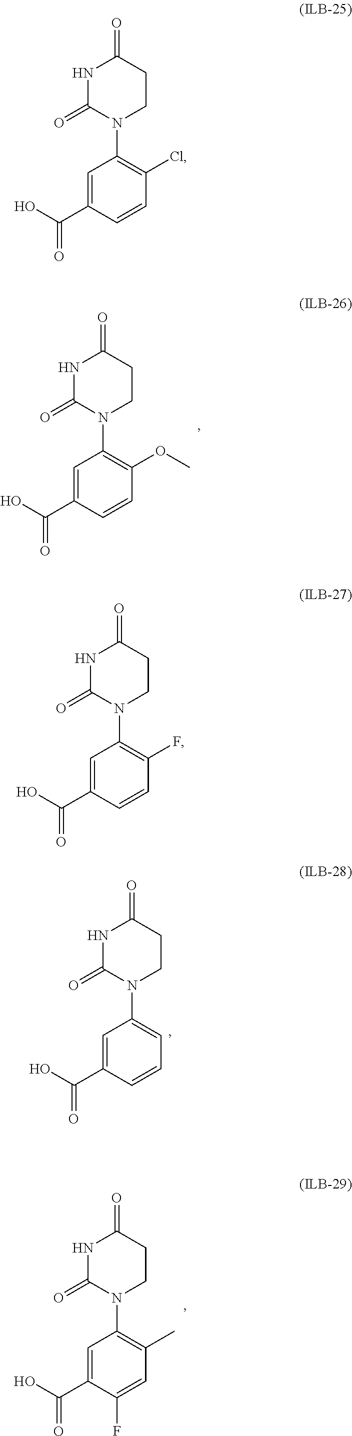 [SMILES: O=C1CCN(c2cc(C(=O)O)ccc2Cl)C(=O)N1 ; COc1ccc(C(=O)O)cc1N1CCC(=O)NC1=O ; O=C1CCN(c2cc(C(=O)O)ccc2F)C(=O)N1 ; O=C1CCN(c2cccc(C(=O)O)c2)C(=O)N1 ; Cc1cc(F)c(C(=O)O)cc1N1CCC(=O)NC1=O]

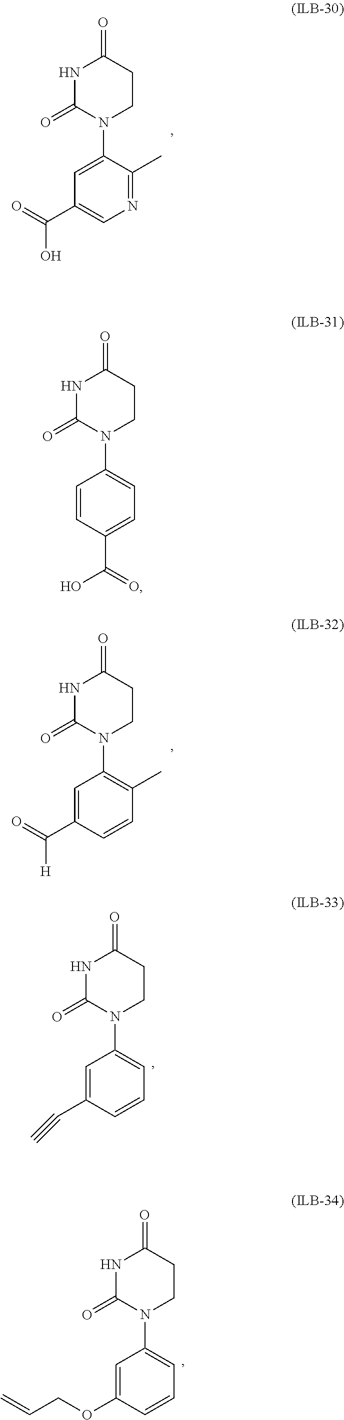

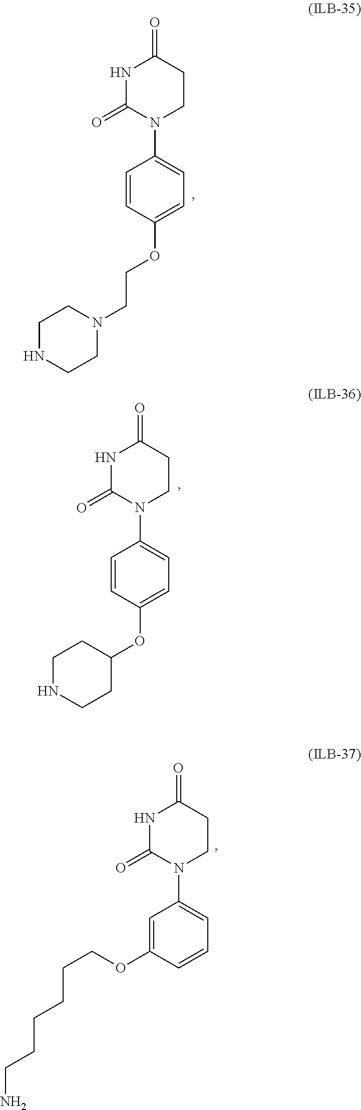 [SMILES: O=C1CCN(c2ccc(OCCN3CCNCC3)cc2)C(=O)N1 ; O=C1CCN(c2ccc(OC3CCNCC3)cc2)C(=O)N1 ; NCCCCCCOc1cccc(N2CCC(=O)NC2=O)c1]

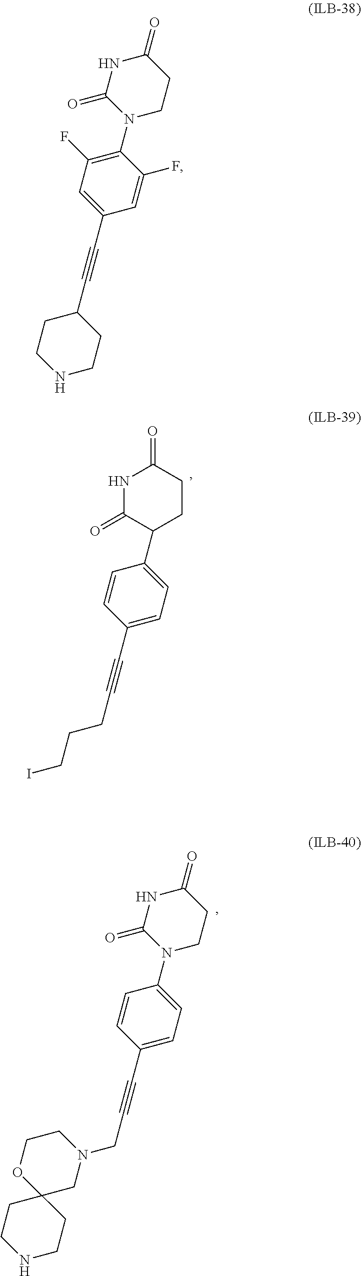 [SMILES: O=C1CCN(c2c(F)cc(C#CC3CCNCC3)cc2F)C(=O)N1 ; O=C1CCC(c2ccc(C#CCCCI)cc2)C(=O)N1 ; O=C1CCN(c2ccc(C#CCN3CCOC4(CCNCC4)C3)cc2)C(=O)N1]

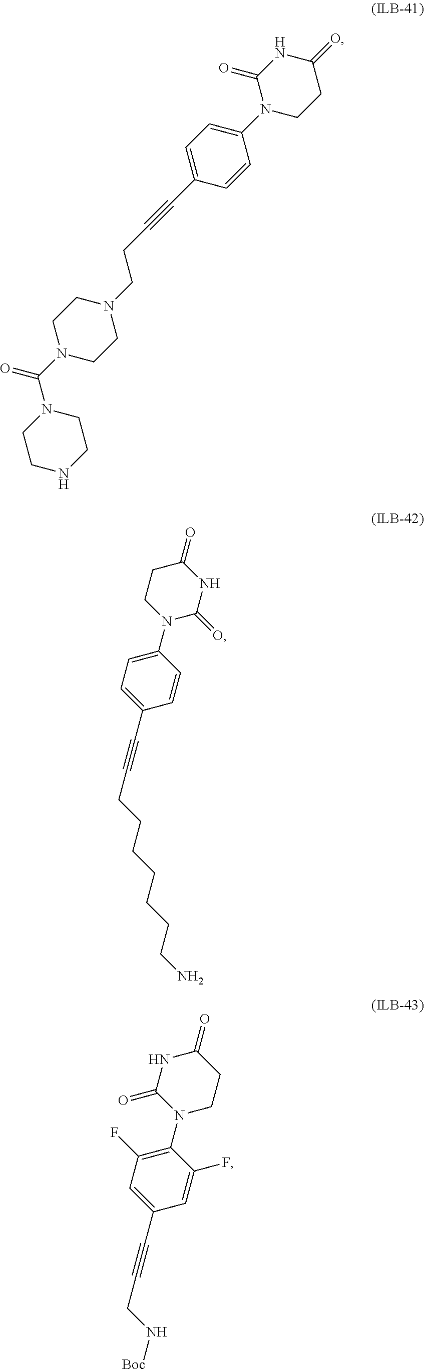 [SMILES: O=C1CCN(c2ccc(C#CCCN3CCN(C(=O)N4CCNCC4)CC3)cc2)C(=O)N1 ; NCCCCCCCC#Cc1ccc(N2CCC(=O)NC2=O)cc1 ; CC(C)(C)OC(=O)NCC#Cc1cc(F)c(N2CCC(=O)NC2=O)c(F)c1]

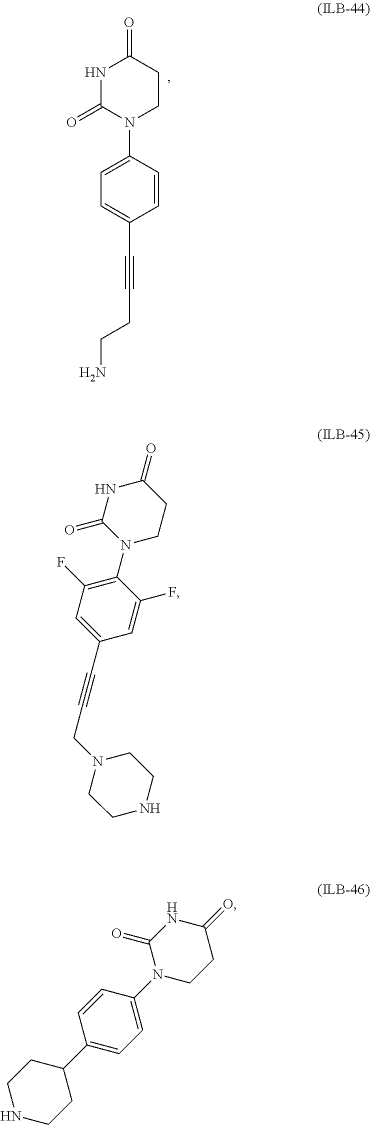 [SMILES: NCCC#Cc1ccc(N2CCC(=O)NC2=O)cc1 ; O=C1CCN(c2c(F)cc(C#CCN3CCNCC3)cc2F)C(=O)N1 ; O=C1CCN(c2ccc(C3CCNCC3)cc2)C(=O)N1]

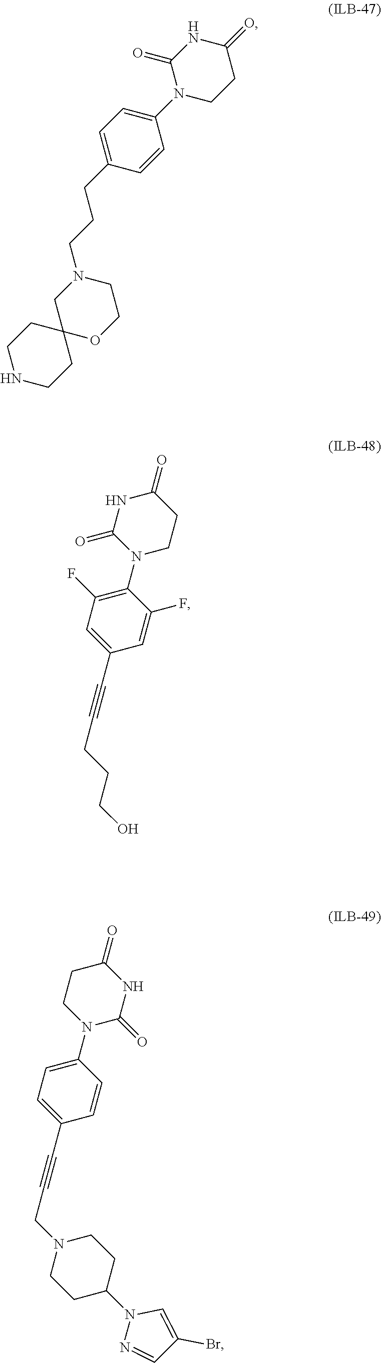 [SMILES: O=C1CCN(c2ccc(CCCN3CCOC4(CCNCC4)C3)cc2)C(=O)N1 ; O=C1CCN(c2c(F)cc(C#CCCCO)cc2F)C(=O)N1 ; O=C1CCN(c2ccc(C#CCN3CCC(n4cc(Br)cn4)CC3)cc2)C(=O)N1]

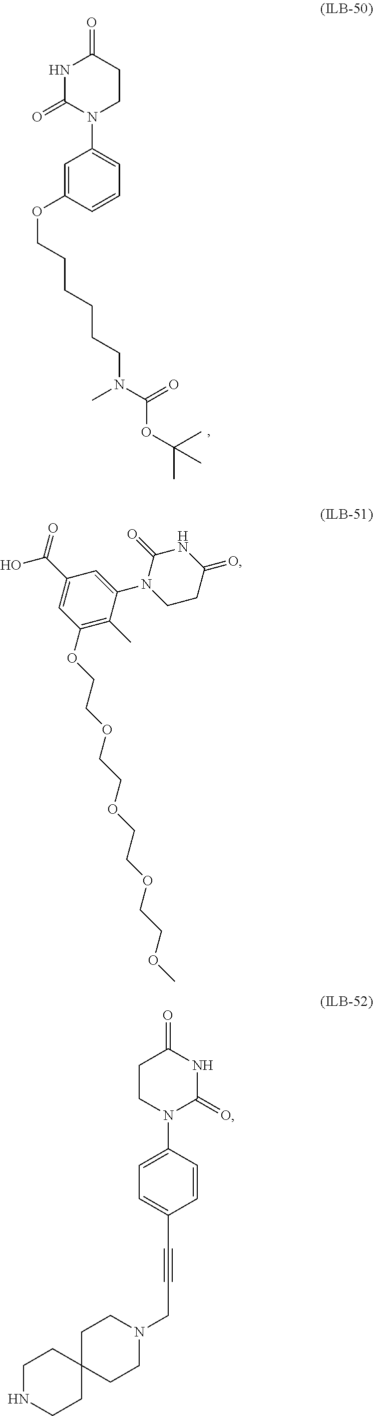

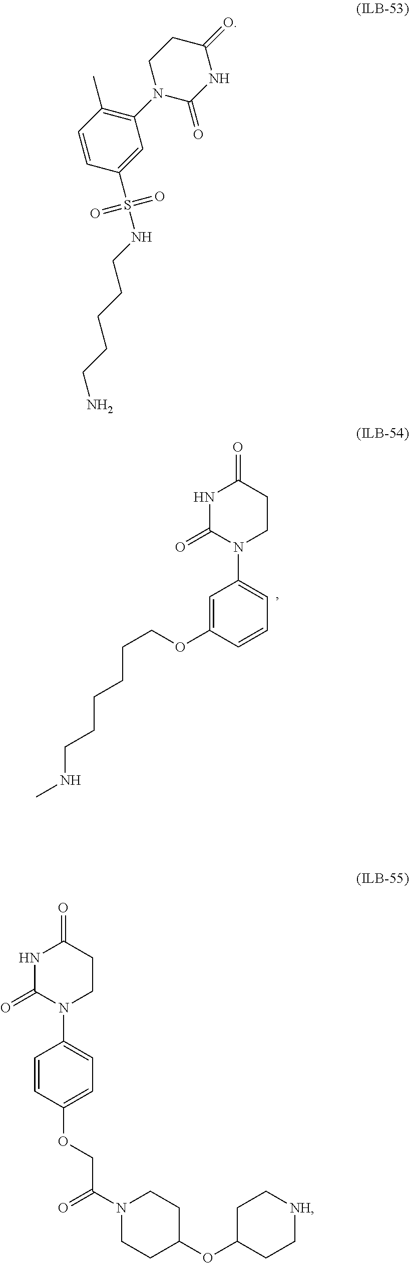 [SMILES: Cc1ccc(S(=O)(=O)NCCCCCN)cc1N1CCC(=O)NC1=O ; CNCCCCCCOc1cccc(N2CCC(=O)NC2=O)c1 ; O=C1CCN(c2ccc(OCC(=O)N3CCC(OC4CCNCC4)CC3)cc2)C(=O)N1]

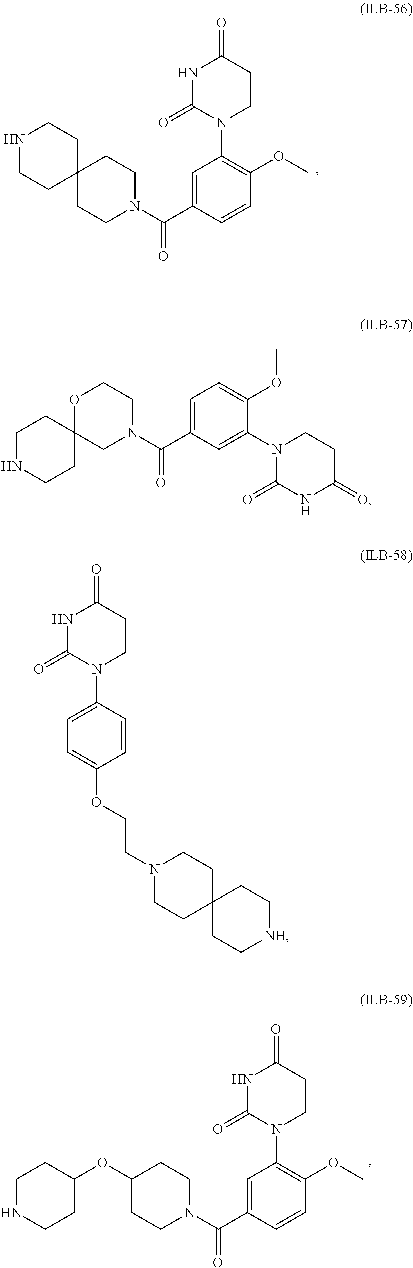 [SMILES: COc1ccc(C(=O)N2CCC3(CCNCC3)CC2)cc1N1CCC(=O)NC1=O ; COc1ccc(C(=O)N2CCOC3(CCNCC3)C2)cc1N1CCC(=O)NC1=O ; O=C1CCN(c2ccc(OCCN3CCC4(CCNCC4)CC3)cc2)C(=O)N1 ; COc1ccc(C(=O)N2CCC(OC3CCNCC3)CC2)cc1N1CCC(=O)NC1=O]

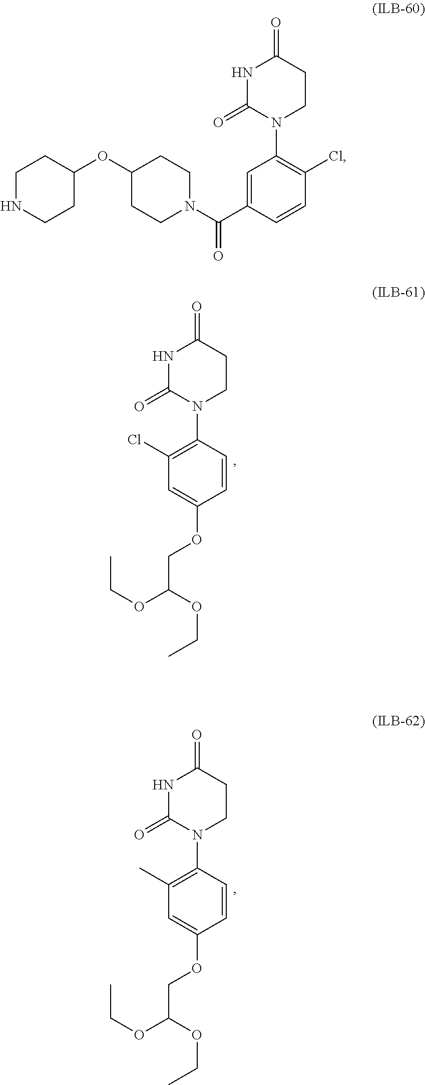 [SMILES: O=C1CCN(c2cc(C(=O)N3CCC(OC4CCNCC4)CC3)ccc2Cl)C(=O)N1 ; CCOC(COc1ccc(N2CCC(=O)NC2=O)c(Cl)c1)OCC ; CCOC(COc1ccc(N2CCC(=O)NC2=O)c(C)c1)OCC]

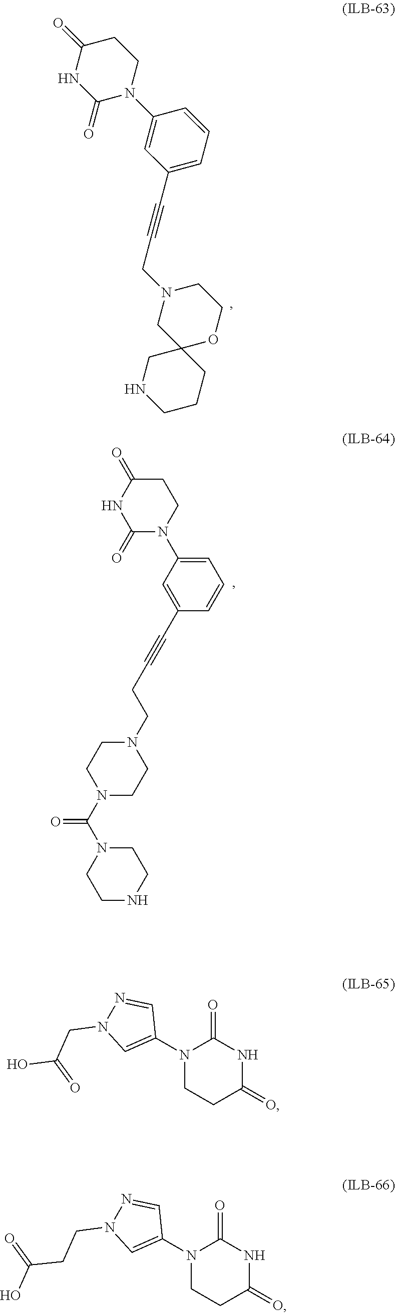 [SMILES: O=C1CCN(c2cccc(C#CCN3CCOC4(CCCNC4)C3)c2)C(=O)N1 ; O=C1CCN(c2cccc(C#CCCN3CCN(C(=O)N4CCNCC4)CC3)c2)C(=O)N1 ; O=C(O)Cn1cc(N2CCC(=O)NC2=O)cn1 ; O=C(O)CCn1cc(N2CCC(=O)NC2=O)cn1]

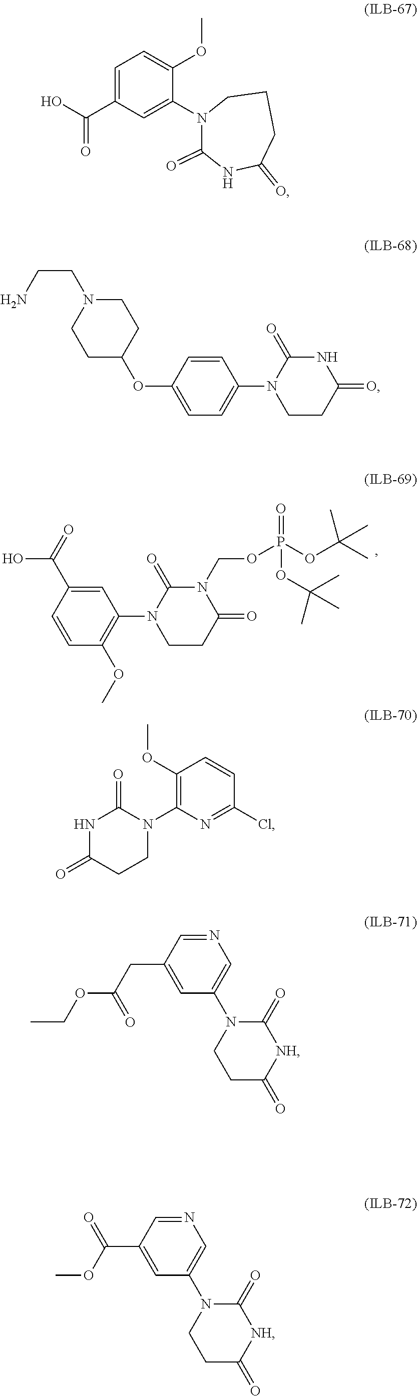

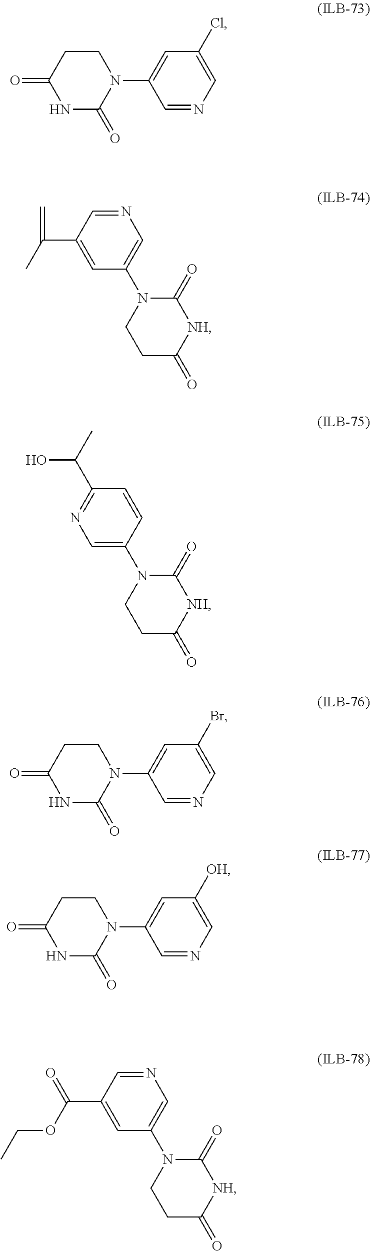 [SMILES: O=C1CCN(c2cncc(Cl)c2)C(=O)N1 ; C=C(C)c1cncc(N2CCC(=O)NC2=O)c1 ; CC(O)c1ccc(N2CCC(=O)NC2=O)cn1 ; O=C1CCN(c2cncc(Br)c2)C(=O)N1 ; O=C1CCN(c2cncc(O)c2)C(=O)N1 ; CCOC(=O)c1cncc(N2CCC(=O)NC2=O)c1]

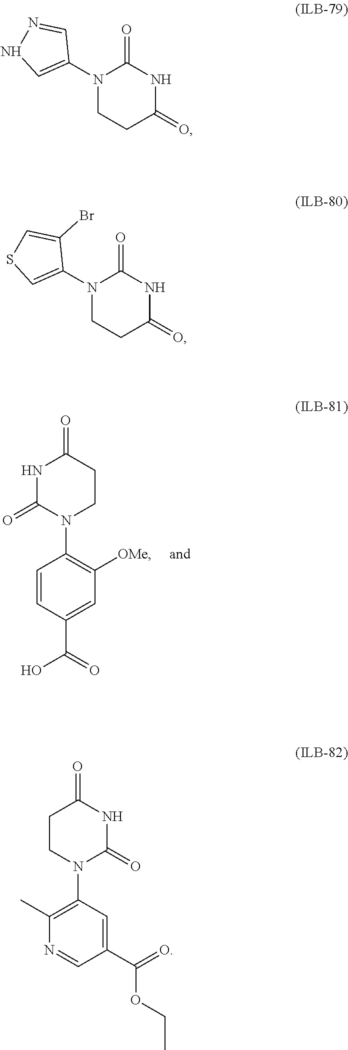 [SMILES: O=C1CCN(c2cn[nH]c2)C(=O)N1 ; O=C1CCN(c2cscc2Br)C(=O)N1 ; COc1cc(C(=O)O)ccc1N1CCC(=O)NC1=O ; CCOC(=O)c1cnc(C)c(N2CCC(=O)NC2=O)c1]

Another embodiment is a compound of Formula (ILB-IV):

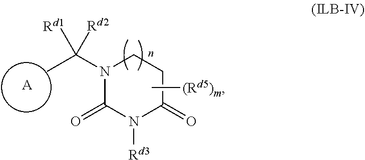

or a pharmaceutically acceptable salt, hydrate, solvate, prodrug, stereoisomer, or tautomer thereof, wherein:
Ring A is

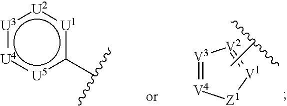

[symbol] denotes the point of attachment to the base molecule of (ILB-IV);
U¹, U², U³, U⁴, and U⁵ are each independently N or CR^{d4} or CR^{L2}, wherein no more than three of U¹, U², U³, U⁴, and U⁵ can be N, and wherein one of U¹, U², U³, U⁴, and U⁵ is CR^{L2} and the remaining are CR^{d4};
Z¹ is selected from the group consisting of O, S, NR^{d4a}; or NR^{L2a}
V¹, V², V³, and V⁴ are each independently N or C, wherein no more than two of V¹, V², V³, and V⁴ can be N, and wherein one of Z¹, V¹, V², V³, and V⁴ is substituted with R^{L2} and the remaining are substituted with R^{d4};
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is H, —CH₂OC(O)R^{P}, —CH₂OP(O)OHOR^{P}, —CH₂OP(O)(R^{P})₂, and —CH₂OP(O)(OR^{p})₂;
each R^{d4} is independently selected from the group consisting of H, hydroxyl, oxo, polyethylene glycol (PEG), halogen, C₁₋₃ alkyl, C₁₋₃ alkoxyl, C₁₋₆ haloalkyl, C₁₋₆ heteroalkyl, and —OC₁₋₇ heteroalkyl;
each R^{d4a} is independently selected from the group consisting of H, polyethylene glycol (PEG), C₁₋₃ alkyl, C₃₋₆ cycloalkyl, C₂₋₆ haloalkyl, and C₂₋₆ heteroalkyl;
each R^{d5} is independently selected from the group consisting of H, C₁₋₆ alkyl, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
R^{L2} is selected from the group consisting of hydroxyl, C₂₋₆ alkenyl, C₂₋₆ alkynyl, C₂₋₆ hydroxyalkyl, —(CH₂)₂₋₆NHR^{c}, —(CH₂)₂₋₆NR^{c}R^{d}, —O—(CH₂)₂₋₆NHR^{c}, C₄₋₈ heteroalkyl, C₂₋₆ haloalkyl, —SO₂—NH—(CH₂)₂₋₆NHR^{c}, —(CH₂)₀₋₃C(O)OH, —(CH₂)₀₋₃C(O)OR^{c}, —O—C₂₋₆ alkenyl, —O—(CH₂)₀₋₃C(O)H, —(CH₂)₀₋₃C(O)H, —O—(CH₂)₁₋₃C(O)OH, —(CH₂)₀₋₃ heterocyclyl, —C(O)—(CH₂)₀₋₃ heterocyclyl, —O—(CH₂)₀₋₃ heterocyclyl, —O—(CH₂)₀₋₃C(O)-heterocyclyl, —C₂₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, —(CH₂)₂₋₆N(R^{c})₂, heterocyclyl, heteroaryl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl, wherein the heterocyclyl and heteroaryl is substituted with 0-2 occurrences of halogen;
R^{L2a} is selected from the group consisting of H, hydroxyl, C₂₋₆ alkenyl, C₂₋₆ alkynyl, C₂₋₆ hydroxyalkyl, —(CH₂)₂₋₆NHR^{c}, —(CH₂)₂₋₆NR^{c}R^{d}, —O—(CH₂)₂₋₆NHR^{c}, C₁₋₈ heteroalkyl, C₁₋₆ haloalkyl, —SO₂—NH—(CH₂)₂₋₆NHR^{c}, —(CH₂)₀₋₃C(O)OH, —(CH₂)₀₋₃C(O)OR^{c}, —O—C₂₋₆ alkenyl, —O—(CH₂)₀₋₃C(O)H, —(CH₂)₀₋₃C(O)H, —O—(CH₂)₁₋₃C(O)OH, —(CH₂)₀₋₃ heterocyclyl, —C(O)—(CH₂)₀₋₃ heterocyclyl, —O—(CH₂)₀₋₃ heterocyclyl, —O—(CH₂)₀₋₃C(O)-heterocyclyl, —C₂₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, —(CH₂)₂₋₆N(R^{c})₂, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl, wherein the heterocyclyl is substituted with 0-2 occurrences of halogen;
R^{c} is H, C₁₋₄ alkyl, C₁₋₆ heteroalkyl, and —C(O)OC₁₋₆ alkyl;
R^{d} is H or C₁₋₄ alkyl; or R^{c} and R^{d} together with the nitrogen atom to which they are attached form a heterocyclyl substituted with 0-2 occurrences of —O-heterocyclyl,
R^{p} is H or C₁₋₆ alkyl;
m is 1 or 2; and
n is 1 or 2.

Another embodiment is a compound of Formula (ILB-IV):

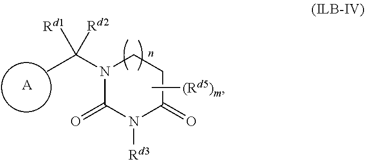

or a pharmaceutically acceptable salt, hydrate, solvate, prodrug, stereoisomer, or tautomer thereof, wherein:
Ring A is selected from the group consisting of:

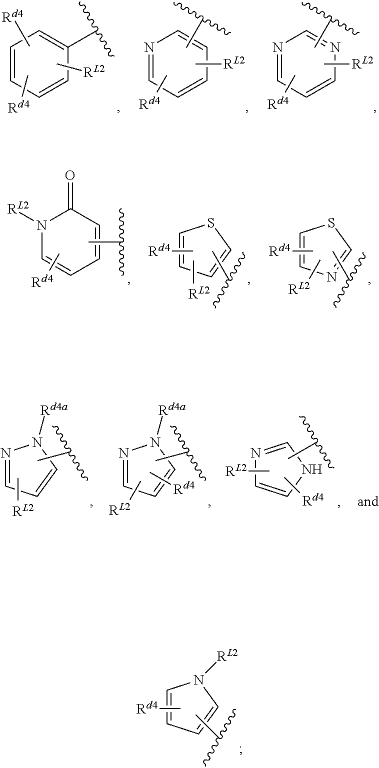

[symbol] denotes the point of attachment to the base molecule of (ILB-IV);
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, hydroxyl, oxo, polyethylene glycol (PEG), halogen, C₁₋₃ alkyl, C₃₋₆ cycloalkyl C₁₋₃ alkoxyl, C₁₋₆ haloalkyl, C₁₋₆ heteroalkyl, and —OC₁₋₇ heteroalkyl;
each R^{d4a} is independently selected from the group consisting of H, polyethylene glycol (PEG), C₁₋₃ alkyl, C₃₋₆ cycloalkyl, C₂₋₆ haloalkyl, and C₂₋₆ heteroalkyl;
each R^{d5} is independently selected from the group consisting of H, C₁₋₆ alkyl, C₁₋₆ haloalkyl, and C₁₋₆ heteroalkyl;
   or two R^{d8} together with the carbon atoms to which they are attached form a cycloalkyl;
   or two R^{d8} attached to the same carbon atom form a C₃₋₄ spirocycloalkyl;
R^{L2} is selected from the group consisting of hydroxyl, halogen, C₂₋₆ alkyl, C₁₋₃ alkoxyl, C₂₋₆ alkenyl, C₂₋₆ alkynyl, C₂₋₆ hydroxyalkyl, —(CH₂)₂₋₆NHR^{c}, —(CH₂)₀₋₆NR^{c}R^{d}, —O—(CH₂)₂₋₆NHR^{c}, C₃₋₈ heteroalkyl, C₁₋₆ haloalkyl, —SO₂—NH—(CH₂)₂₋₆NHR^{c}, —(CH₂)₀₋₃C(O)OH, —O—(CH₂)₁₋₃C(O)H, —(CH₂)₁₋₃C(O)H, —O—(CH₂)₁₋₃C(O)OH, —(CH₂)₀₋₃C₃₋₇ carbocyclyl, —(CH₂)₀₋₃ heterocyclyl, —C(O)—(CH₂)₀₋₃ heterocyclyl, —O—(CH₂)₀₋₃ heterocyclyl, —C₂₋₆ alkynyl-heterocyclyl, —C₂₋₆ alkynyl-heterocyclyl-heteroaryl, C₆ aryl, and heteroaryl, wherein the alkynyl, alkoxyl, heterocyclyl, heteroalkyl, carbocyclyl, aryl, and heteroaryl is substituted with 0-2 occurrences of halogen, hydroxyl, —(CH₂)₀₋₃C(O)H, —C(O)O-benzyl, —(CH₂)₂₋₆NHR^{c}, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{p} is H or C₁₋₆ alkyl;
m is 1 or 2; and
n is 1 or 2.

In an embodiment, Ring A is selected from the group consisting of

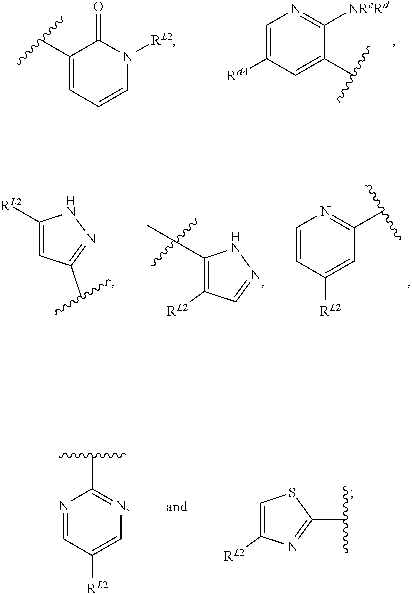

wherein R^{c} is H, C₁₋₄ alkyl, C₁₋₆ heteroalkyl, and —C(O)OC₁₋₆ alkyl; and R^{d} is H or C₁₋₄ alkyl; or R^{c} and R^{d} together with the nitrogen atom to which they are attached form a heterocyclyl substituted with 0-2 occurrences of —O-heterocyclyl.

In an embodiment, R^{d4} is H or halogen.

In an embodiment, each R^{d4a} is independently H.

In an embodiment, R^{L2} is selected from the group consisting of halogen, —(CH₂)₀₋₆NR^{c}R^{d}, C₁₋₆ haloalkyl, —(CH₂)₀₋₃C(O)OH, —(CH₂)₀₋₃ heterocyclyl, and —C(O)O-benzyl.

In an embodiment, R^{c} is H, C₁₋₄ alkyl, or —C(O)OC₁₋₆ alkyl.

In an embodiment, R^{d} is H or C₁₋₄ alkyl.

Another embodiment is a compound or a pharmaceutically acceptable salt, hydrate, solvate, prodrug, stereoisomer, or tautomer thereof, selected from:

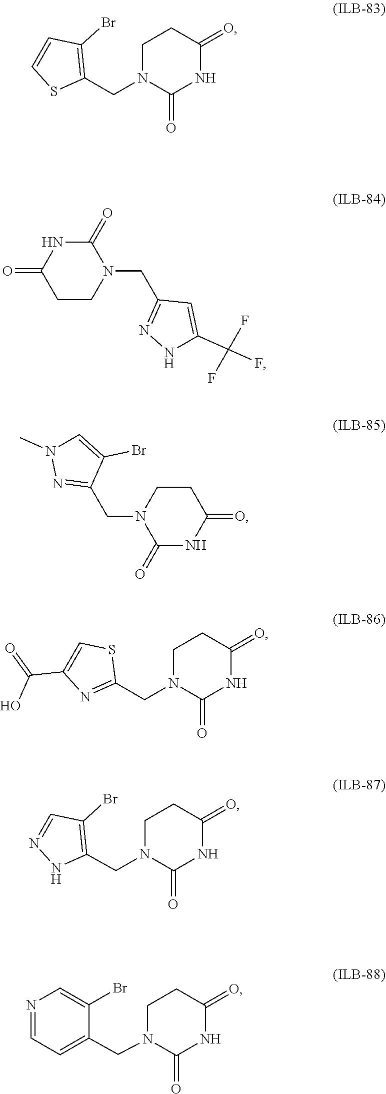 [SMILES: O=C1CCN(Cc2sccc2Br)C(=O)N1 ; O=C1CCN(Cc2cc(C(F)(F)F)[nH]n2)C(=O)N1 ; Cn1cc(Br)c(CN2CCC(=O)NC2=O)n1 ; O=C1CCN(Cc2nc(C(=O)O)cs2)C(=O)N1 ; O=C1CCN(Cc2[nH]ncc2Br)C(=O)N1 ; O=C1CCN(Cc2ccncc2Br)C(=O)N1]

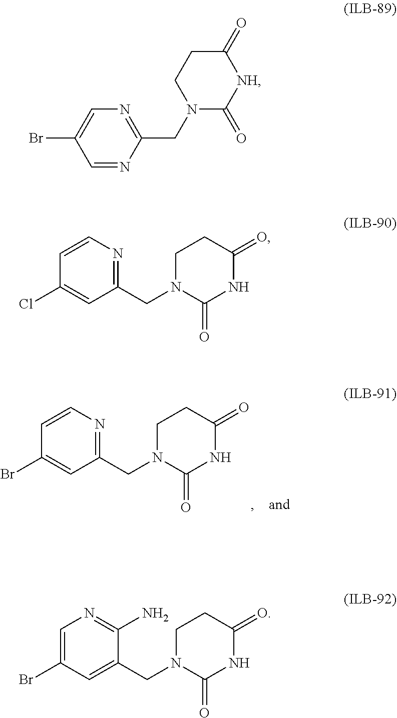 [SMILES: O=C1CCN(Cc2ncc(Br)cn2)C(=O)N1 ; O=C1CCN(Cc2cc(Cl)ccn2)C(=O)N1 ; O=C1CCN(Cc2cc(Br)ccn2)C(=O)N1 ; Nc1ncc(Br)cc1CN1CCC(=O)NC1=O]

### Compounds

Another embodiment is a bifunctional compound of Formula (II):

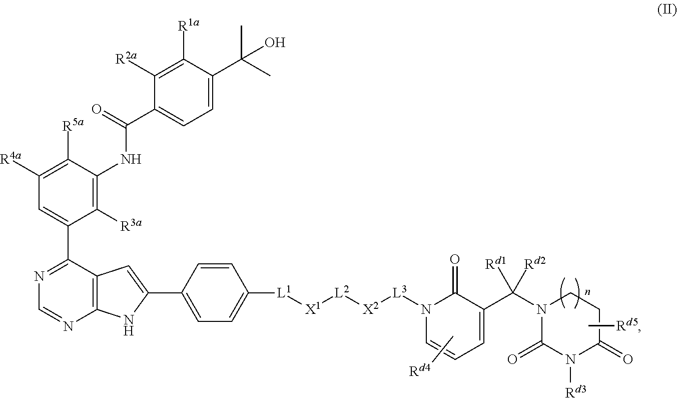

or a pharmaceutically acceptable salt, hydrate, solvate, prodrug, stereoisomer, or tautomer thereof, wherein:
R^{1a} is H or halo;
R^{2a} is halo;
R^{3a} is C₁₋₆ alkyl;
R^{4a} is halo;
R^{5a} is H or halo;
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-III);
wherein no more than 2 of L¹, X¹, X², L², and L³ can simultaneously be a bond;
R′ is hydrogen or C₁₋₆ alkyl;
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl; and
R^{p} is H or C₁₋₆ alkyl.

In an embodiment, R^{2a} is fluoro. In an embodiment, R^{3a} is C₁₋₃ alkyl. In an embodiment, R^{3a} is methyl.

In an embodiment, R^{4a} is fluoro. In an embodiment, L¹ is C₁₋₉ alkylene. In an embodiment, —X¹-L²-X²— is:

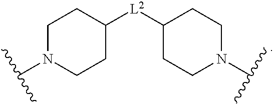

In an embodiment, L² is —C(O)—, —O—, or C₁₋₆ alkylene. In an embodiment, L³ is selected from the group consisting of a bond, —O—, —C(O)—, —(O)₂—, —C₁₋₆ alkylene, C₂₋₆ alkynylene, and C₁₋₆ heteroalkylene. In an embodiment, R^{d4} is H. In an embodiment, R^{d1} is H. In an embodiment, R^{d2} is H. In an embodiment, R^{d1} and R^{d2} are both H. In an embodiment, n is 1. In an embodiment, R^{d3} is H. In an embodiment, R^{d5} is H or C₁₋₃ alkyl. In an embodiment, R^{d5} is H.

Another embodiment is a bifunctional compound of Formula (IIA):

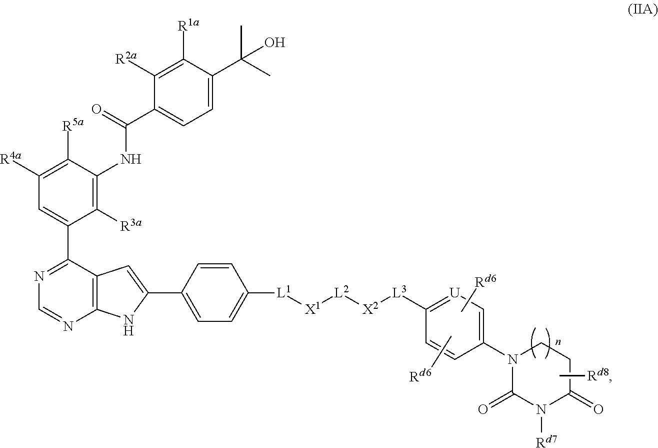

or a pharmaceutically acceptable salt, hydrate, solvate, prodrug, stereoisomer, or tautomer thereof, wherein:
R^{1a} is H or halo;
R^{2a} is halo;
R^{3a} is C₁₋₆ alkyl;
R^{4a} is halo;
R^{5a} is H or halo;
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, *C(O)—C₁₋₆ alkylene-O, and *C(O)—C₁₋₉ heteroalkylene, wherein * denotes the point of attachment of L³ to X² in Formula (BF-III);
wherein no more than 2 of L¹, X¹, X², L², and L³ can simultaneously be a bond;
R′ is hydrogen or C₁₋₆ alkyl;
U is —CR^{d6} or N;
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
U is —CR^{d6} or N;
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d8} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

In an embodiment, R^{2a} is fluoro. In an embodiment, R^{3a} is C₁₋₃ alkyl. In an embodiment, R^{3a} is methyl.

In another aspect, R^{4a} is fluoro. In an embodiment, L¹ is C₁₋₉ alkylene. In an embodiment, —X¹-L²-X²— is:

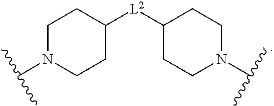

In an embodiment, L² is —C(O)—, —O—, or C₁₋₆ alkylene. In an embodiment, L³ is selected from the group consisting of a bond, —O—, —C(O)—, —S(O)₂—, C₁₋₆ alkylene, C₂₋₆ alkynylene, and C₁₋₆ heteroalkylene. In an embodiment, R^{d4} is H. In an embodiment, R^{d1} is H. In an embodiment, R^{d2} is H. In an embodiment, R^{d1} and R² are both H. In an embodiment, n is 1. In an embodiment, R^{d3} is H. In an embodiment, R^{d5} is H or C₁₋₃ alkyl.
In an embodiment, R^{d5} is H.

Another embodiment is a bifunctional compound, or a pharmaceutically acceptable salt, hydrate, solvate, prodrug, stereoisomer, or tautomer thereof, selected from:

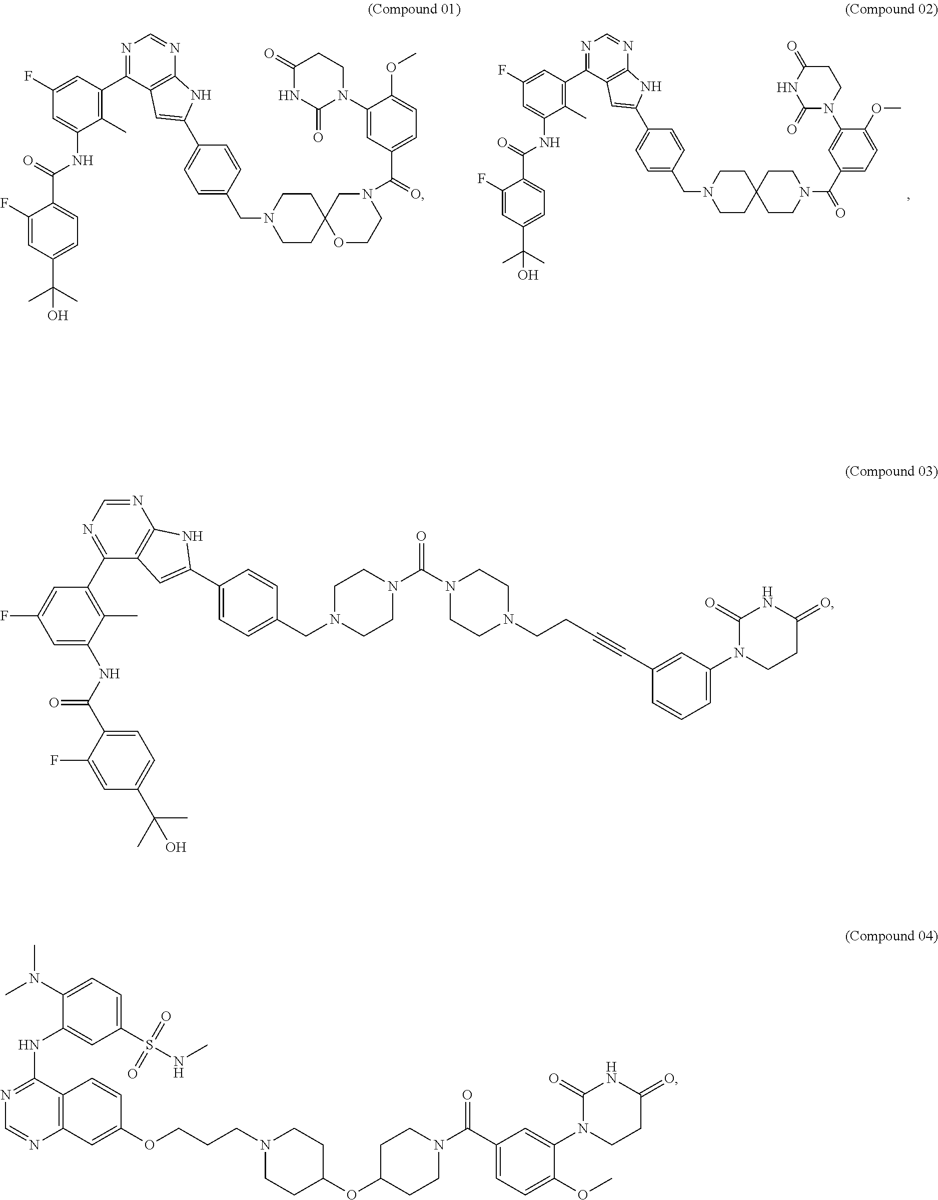 [SMILES: COc1ccc(C(=O)N2CCOC3(CCN(Cc4ccc(-c5cc6c(-c7cc(F)cc(NC(=O)c8ccc(C(C)(C)O)cc8F)c7C)ncnc6[nH]5)cc4)CC3)C2)cc1N1CCC(=O)NC1=O ; COc1ccc(C(=O)N2CCC3(CCN(Cc4ccc(-c5cc6c(-c7cc(F)cc(NC(=O)c8ccc(C(C)(C)O)cc8F)c7C)ncnc6[nH]5)cc4)CC3)CC2)cc1N1CCC(=O)NC1=O ; Cc1c(NC(=O)c2ccc(C(C)(C)O)cc2F)cc(F)cc1-c1ncnc2[nH]c(-c3ccc(CN4CCN(C(=O)N5CCN(CCC#Cc6cccc(N7CCC(=O)NC7=O)c6)CC5)CC4)cc3)cc12 ; CNS(=O)(=O)c1ccc(N(C)C)c(Nc2ncnc3cc(OCCCN4CCC(OC5CCN(C(=O)c6ccc(OC)c(N7CCC(=O)NC7=O)c6)CC5)CC4)ccc23)c1]

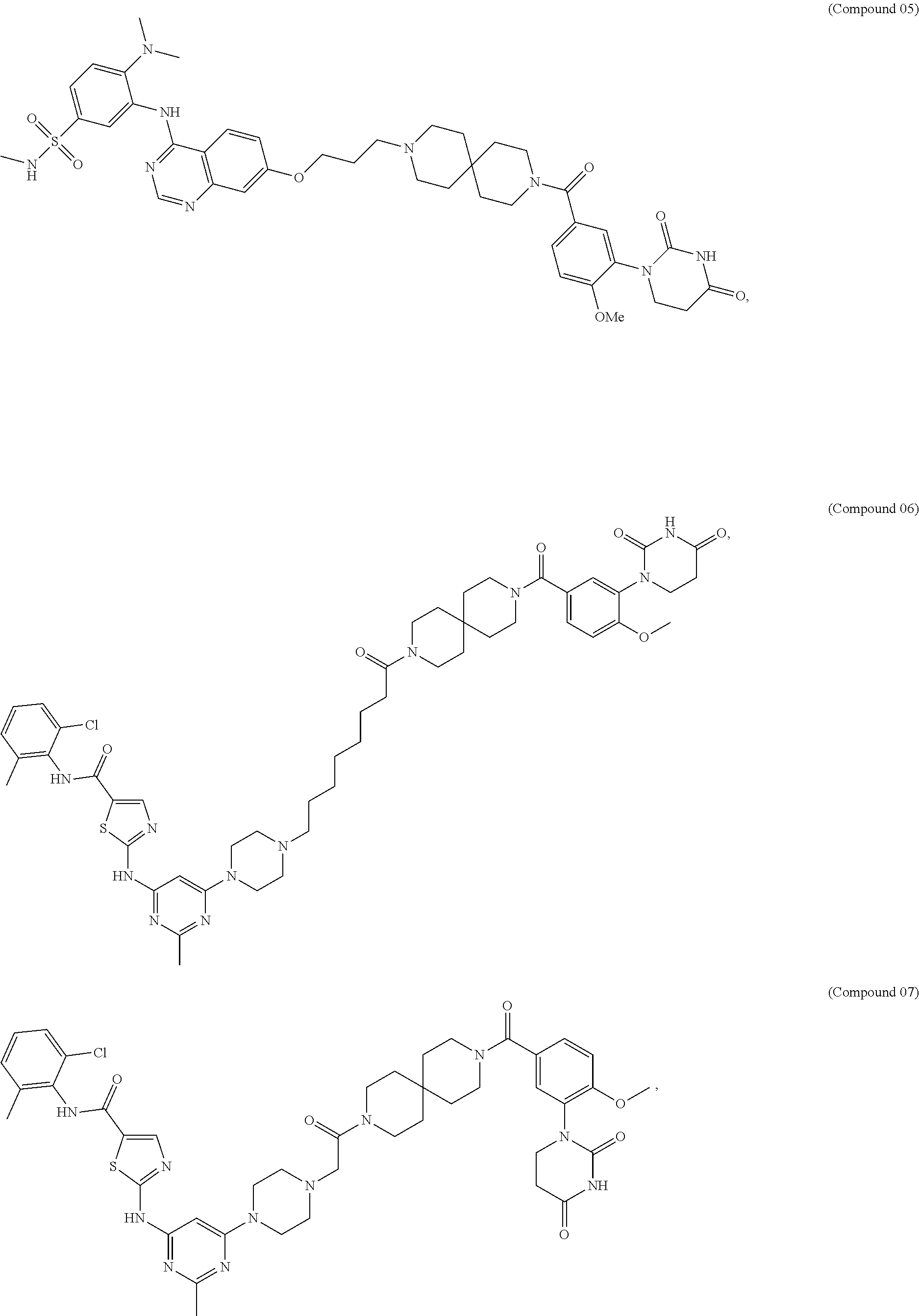 [SMILES: CNS(=O)(=O)c1ccc(N(C)C)c(Nc2ncnc3cc(OCCCN4CCC5(CC4)CCN(C(=O)c4ccc(OC)c(N6CCC(=O)NC6=O)c4)CC5)ccc23)c1 ; COc1ccc(C(=O)N2CCC3(CCN(C(=O)CCCCCCCN4CCN(c5cc(Nc6ncc(C(=O)Nc7c(C)cccc7Cl)s6)nc(C)n5)CC4)CC3)CC2)cc1N1CCC(=O)NC1=O ; COc1ccc(C(=O)N2CCC3(CCN(C(=O)CN4CCN(c5cc(Nc6ncc(C(=O)Nc7c(C)cccc7Cl)s6)nc(C)n5)CC4)CC3)CC2)cc1N1CCC(=O)NC1=O]

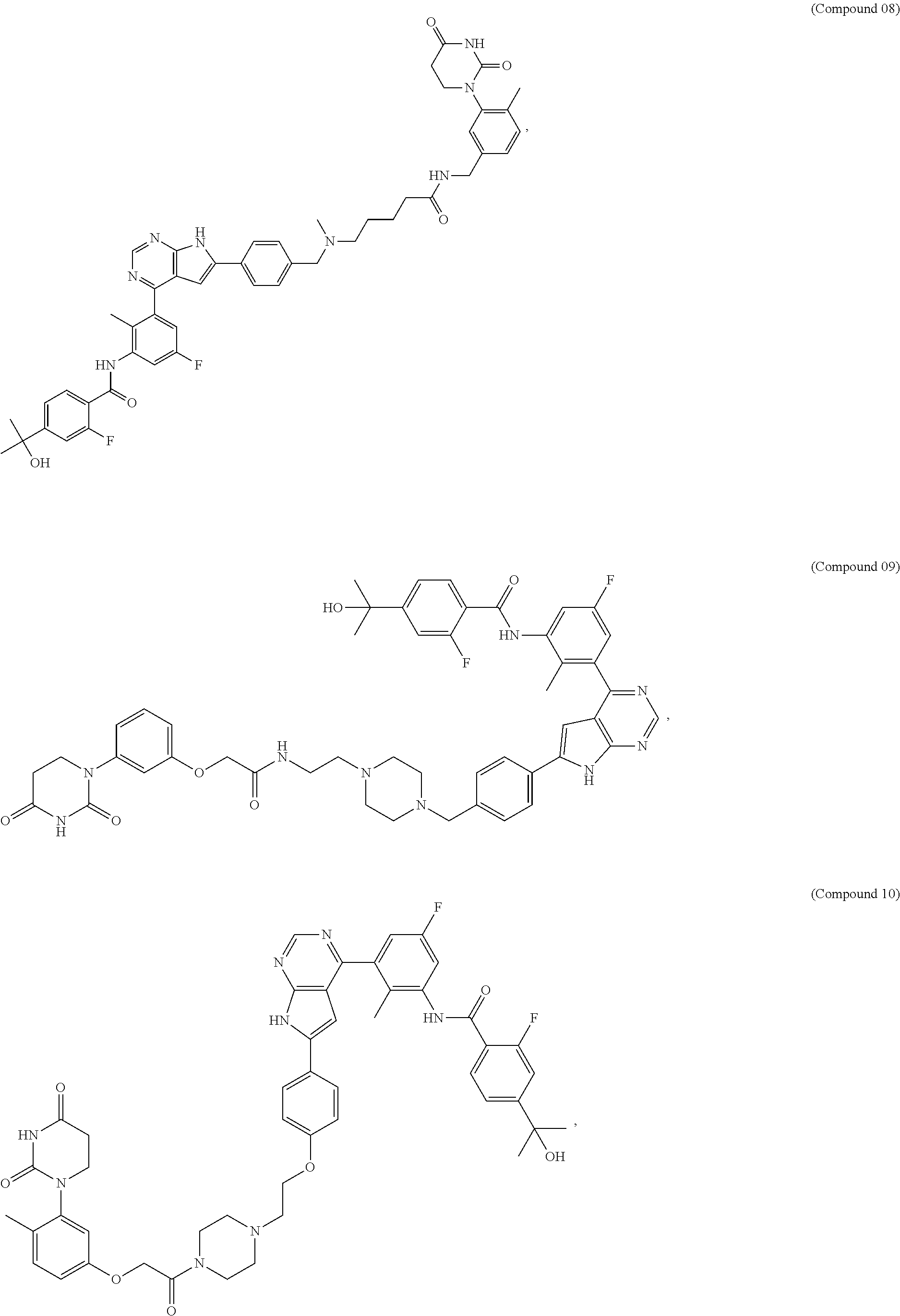 [SMILES: Cc1ccc(CNC(=O)CCCCN(C)Cc2ccc(-c3cc4c(-c5cc(F)cc(NC(=O)c6ccc(C(C)(C)O)cc6F)c5C)ncnc4[nH]3)cc2)cc1N1CCC(=O)NC1=O ; Cc1c(NC(=O)c2ccc(C(C)(C)O)cc2F)cc(F)cc1-c1ncnc2[nH]c(-c3ccc(CN4CCN(CCNC(=O)COc5cccc(N6CCC(=O)NC6=O)c5)CC4)cc3)cc12 ; Cc1ccc(OCC(=O)N2CCN(CCOc3ccc(-c4cc5c(-c6cc(F)cc(NC(=O)c7ccc(C(C)(C)O)cc7F)c6C)ncnc5[nH]4)cc3)CC2)cc1N1CCC(=O)NC1=O]

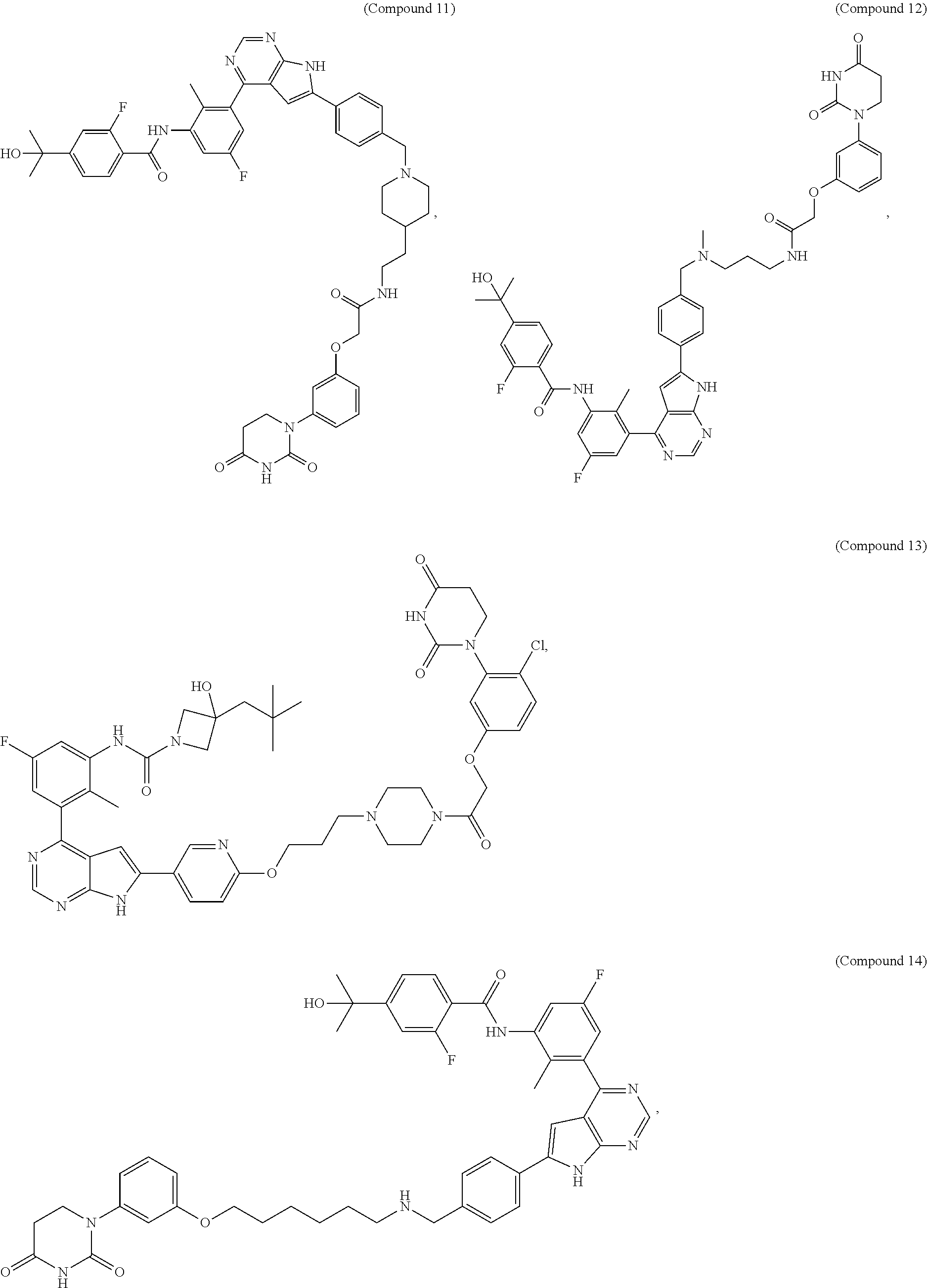 [SMILES: Cc1c(NC(=O)c2ccc(C(C)(C)O)cc2F)cc(F)cc1-c1ncnc2[nH]c(-c3ccc(CN4CCC(CCNC(=O)COc5cccc(N6CCC(=O)NC6=O)c5)CC4)cc3)cc12 ; Cc1c(NC(=O)c2ccc(C(C)(C)O)cc2F)cc(F)cc1-c1ncnc2[nH]c(-c3ccc(CN(C)CCCNC(=O)COc4cccc(N5CCC(=O)NC5=O)c4)cc3)cc12 ; Cc1c(NC(=O)N2CC(O)(CC(C)(C)C)C2)cc(F)cc1-c1ncnc2[nH]c(-c3ccc(OCCCN4CCN(C(=O)COc5ccc(Cl)c(N6CCC(=O)NC6=O)c5)CC4)nc3)cc12 ; Cc1c(NC(=O)c2ccc(C(C)(C)O)cc2F)cc(F)cc1-c1ncnc2[nH]c(-c3ccc(CNCCCCCCOc4cccc(N5CCC(=O)NC5=O)c4)cc3)cc12]

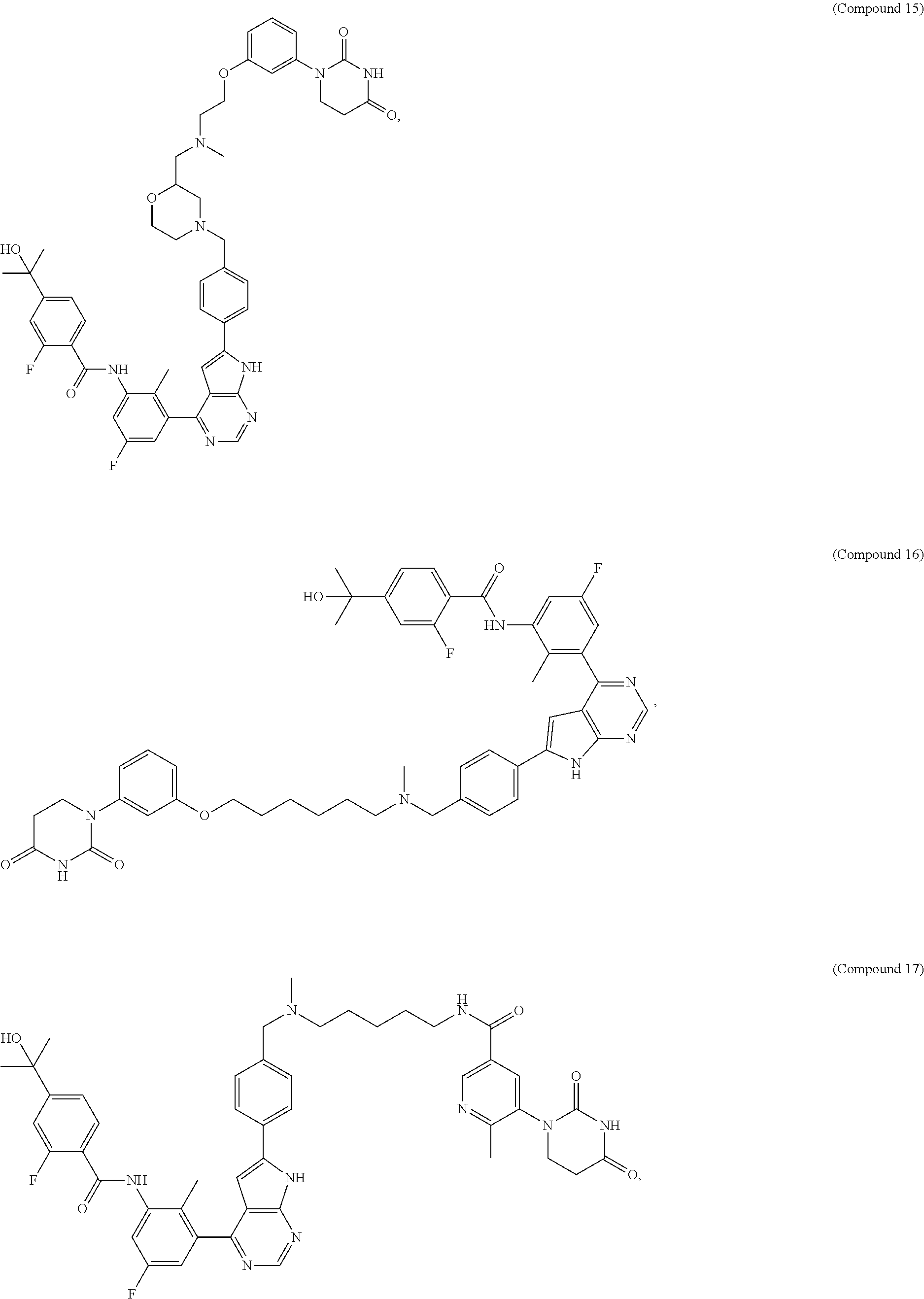 [SMILES: Cc1c(NC(=O)c2ccc(C(C)(C)O)cc2F)cc(F)cc1-c1ncnc2[nH]c(-c3ccc(CN4CCOC(CN(C)CCOc5cccc(N6CCC(=O)NC6=O)c5)C4)cc3)cc12 ; Cc1c(NC(=O)c2ccc(C(C)(C)O)cc2F)cc(F)cc1-c1ncnc2[nH]c(-c3ccc(CN(C)CCCCCCOc4cccc(N5CCC(=O)NC5=O)c4)cc3)cc12 ; Cc1ncc(C(=O)NCCCCCN(C)Cc2ccc(-c3cc4c(-c5cc(F)cc(NC(=O)c6ccc(C(C)(C)O)cc6F)c5C)ncnc4[nH]3)cc2)cc1N1CCC(=O)NC1=O]

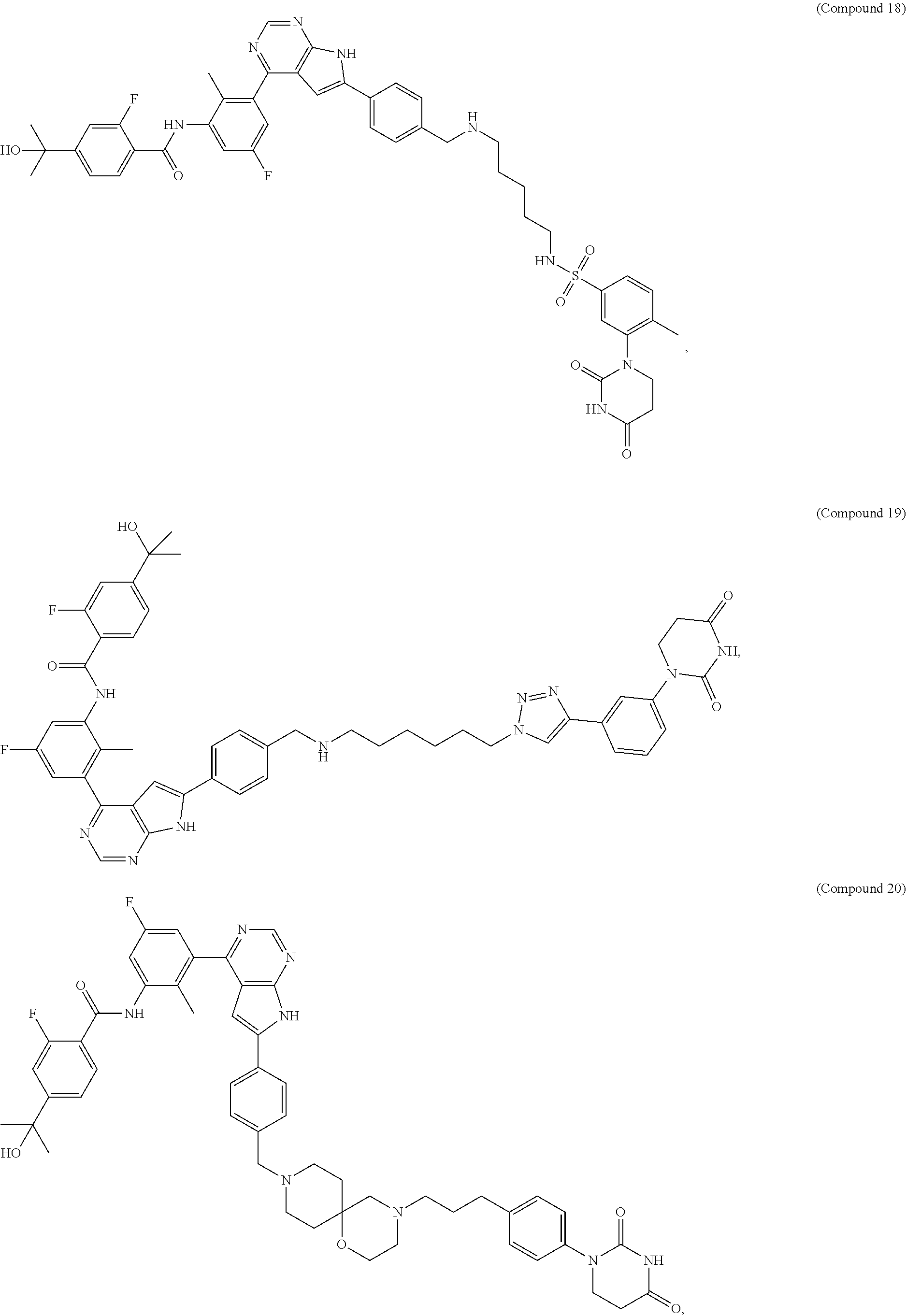

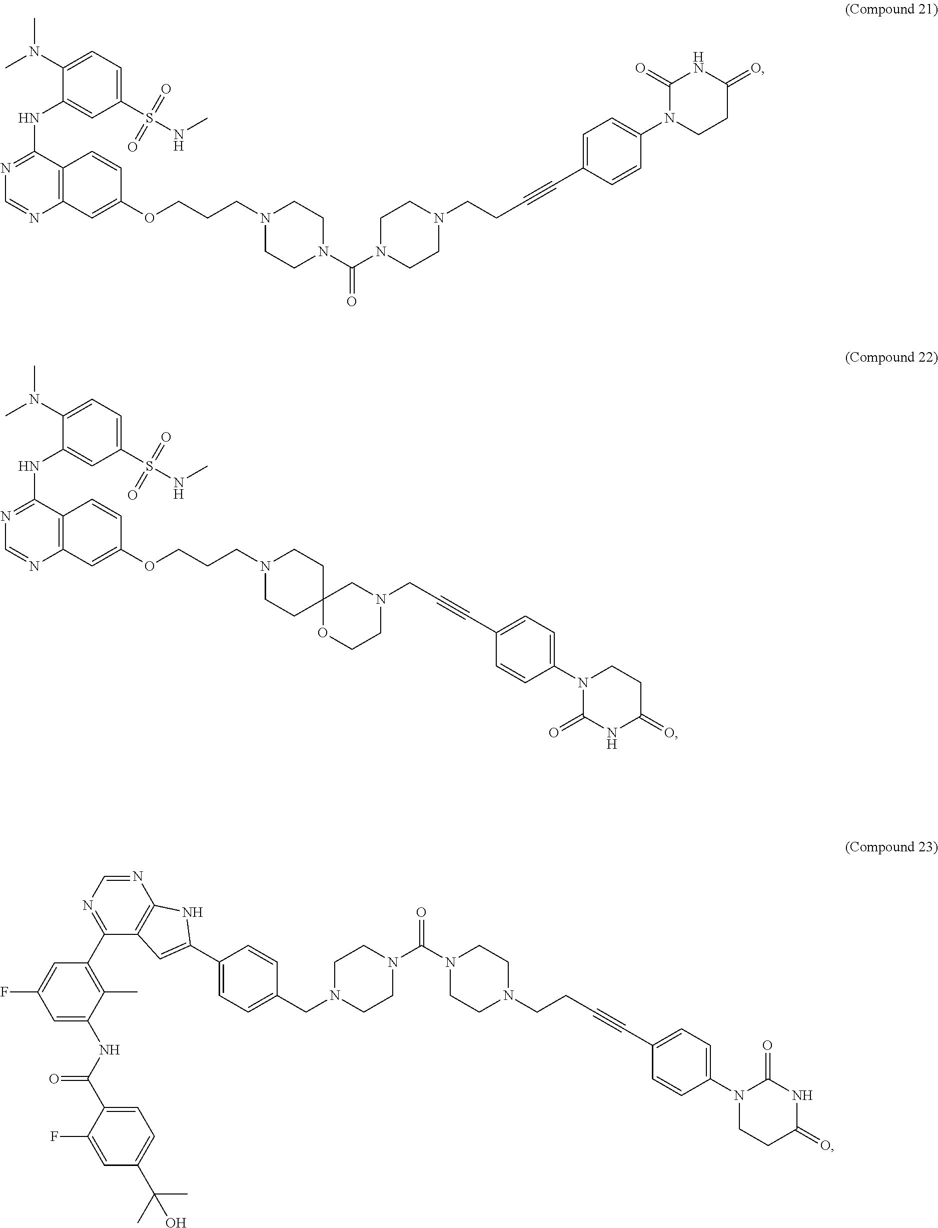 [SMILES: CNS(=O)(=O)c1ccc(N(C)C)c(Nc2ncnc3cc(OCCCN4CCN(C(=O)N5CCN(CCC#Cc6ccc(N7CCC(=O)NC7=O)cc6)CC5)CC4)ccc23)c1 ; CNS(=O)(=O)c1ccc(N(C)C)c(Nc2ncnc3cc(OCCCN4CCC5(CC4)CN(CC#Cc4ccc(N6CCC(=O)NC6=O)cc4)CCO5)ccc23)c1 ; Cc1c(NC(=O)c2ccc(C(C)(C)O)cc2F)cc(F)cc1-c1ncnc2[nH]c(-c3ccc(CN4CCN(C(=O)N5CCN(CCC#Cc6ccc(N7CCC(=O)NC7=O)cc6)CC5)CC4)cc3)cc12]

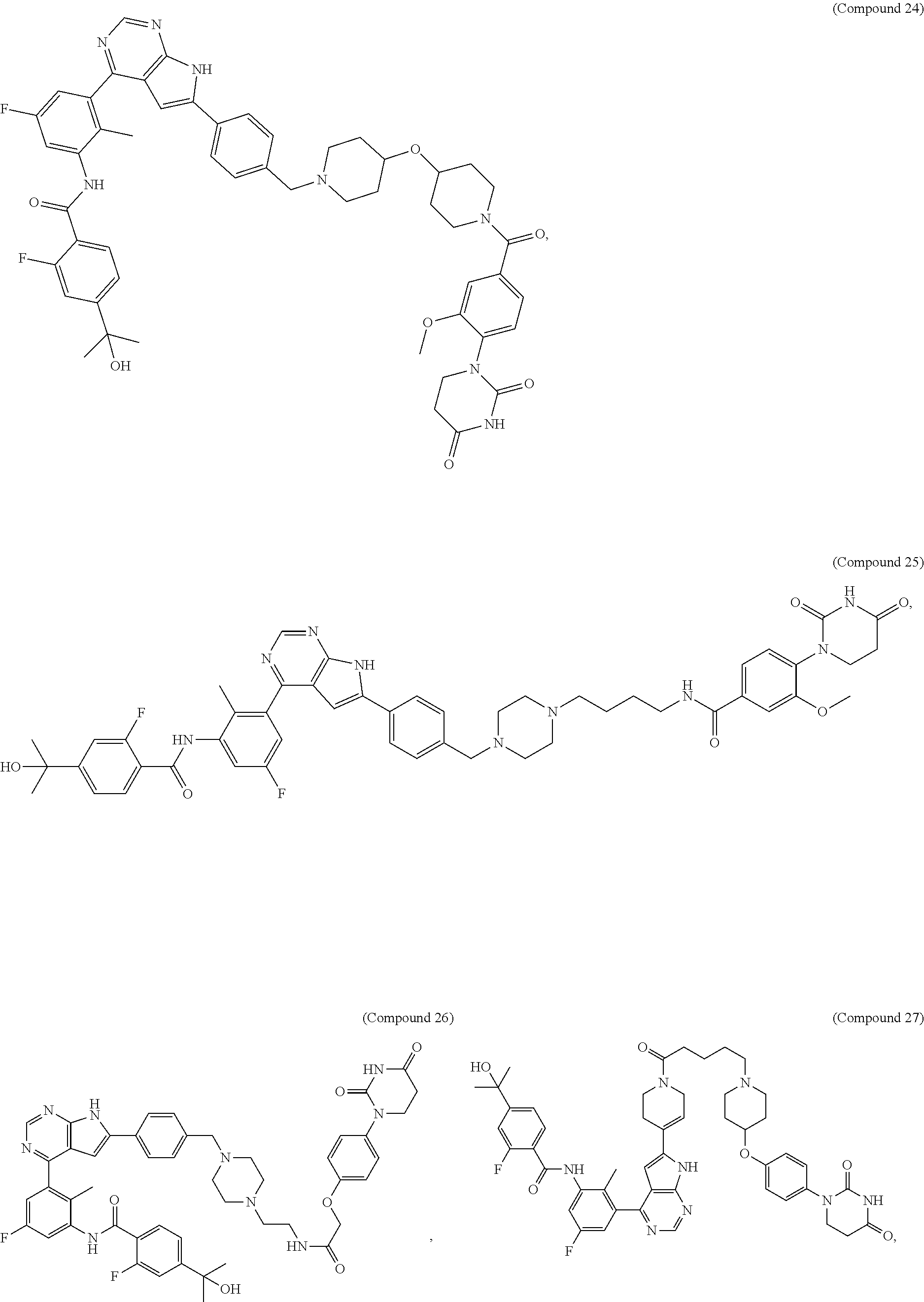 [SMILES: COc1cc(C(=O)N2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O ; COc1cc(C(=O)NCCCCN2CCN(Cc3ccc(-c4cc5c(-c6cc(F)cc(NC(=O)c7ccc(C(C)(C)O)cc7F)c6C)ncnc5[nH]4)cc3)CC2)ccc1N1CCC(=O)NC1=O ; Cc1c(NC(=O)c2ccc(C(C)(C)O)cc2F)cc(F)cc1-c1ncnc2[nH]c(-c3ccc(CN4CCN(CCNC(=O)COc5ccc(N6CCC(=O)NC6=O)cc5)CC4)cc3)cc12 ; Cc1c(NC(=O)c2ccc(C(C)(C)O)cc2F)cc(F)cc1-c1ncnc2[nH]c(C3=CCN(C(=O)CCCCN4CCC(Oc5ccc(N6CCC(=O)NC6=O)cc5)CC4)CC3)cc12]

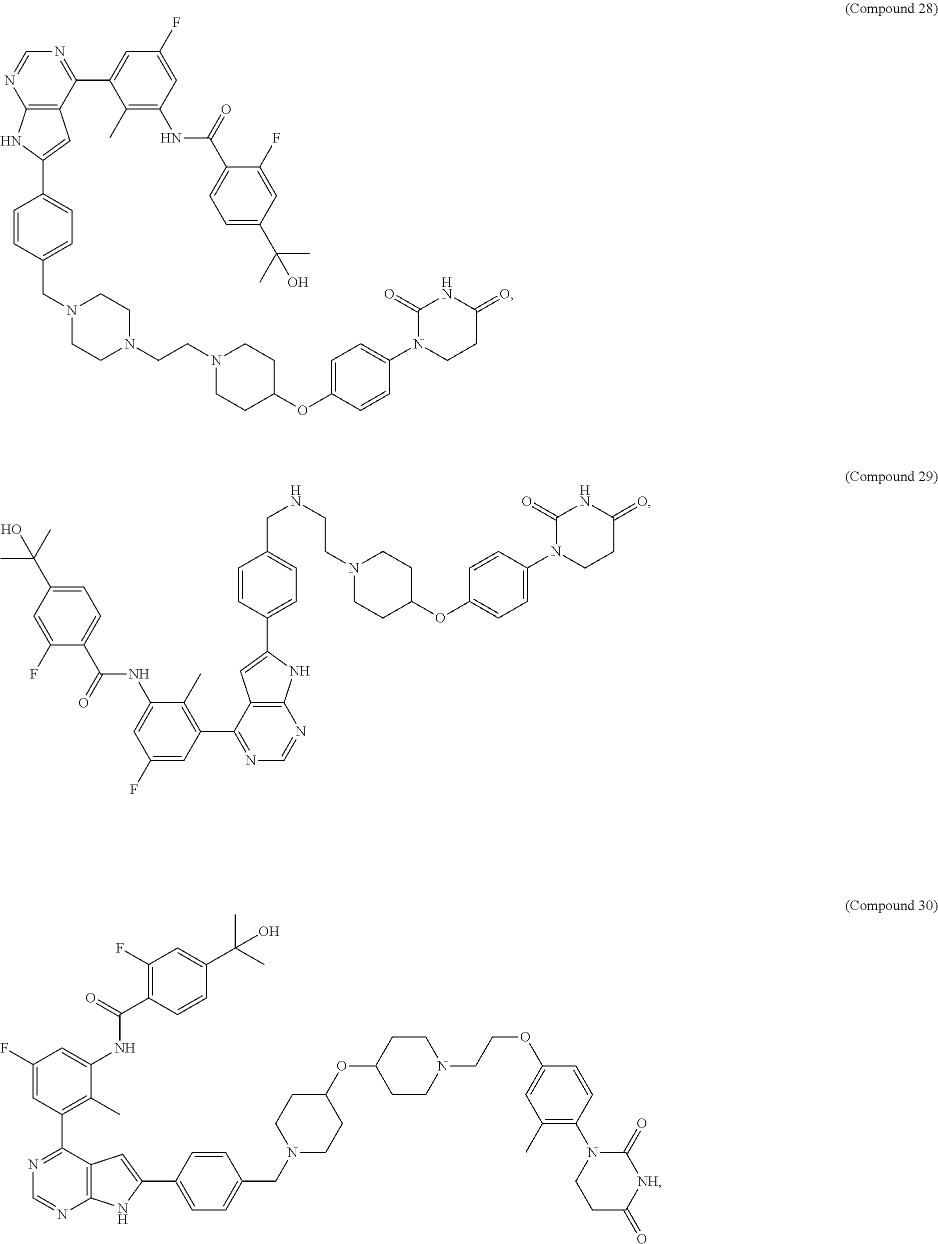 [SMILES: Cc1c(NC(=O)c2ccc(C(C)(C)O)cc2F)cc(F)cc1-c1ncnc2[nH]c(-c3ccc(CN4CCN(CCN5CCC(Oc6ccc(N7CCC(=O)NC7=O)cc6)CC5)CC4)cc3)cc12 ; Cc1c(NC(=O)c2ccc(C(C)(C)O)cc2F)cc(F)cc1-c1ncnc2[nH]c(-c3ccc(CNCCN4CCC(Oc5ccc(N6CCC(=O)NC6=O)cc5)CC4)cc3)cc12 ; Cc1cc(OCCN2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O]

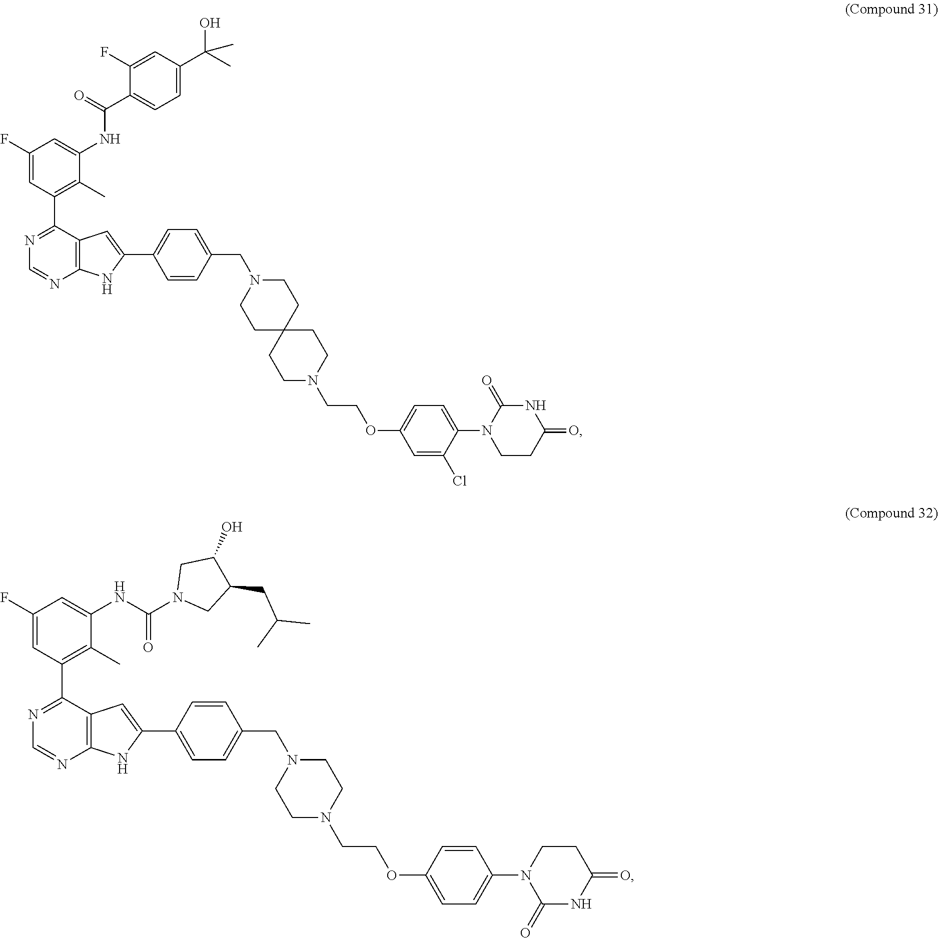 [SMILES: Cc1c(NC(=O)c2ccc(C(C)(C)O)cc2F)cc(F)cc1-c1ncnc2[nH]c(-c3ccc(CN4CCC5(CCN(CCOc6ccc(N7CCC(=O)NC7=O)c(Cl)c6)CC5)CC4)cc3)cc12 ; Cc1c(NC(=O)N2C[C@H](CC(C)C)[C@@H](O)C2)cc(F)cc1-c1ncnc2[nH]c(-c3ccc(CN4CCN(CCOc5ccc(N6CCC(=O)NC6=O)cc5)CC4)cc3)cc12]

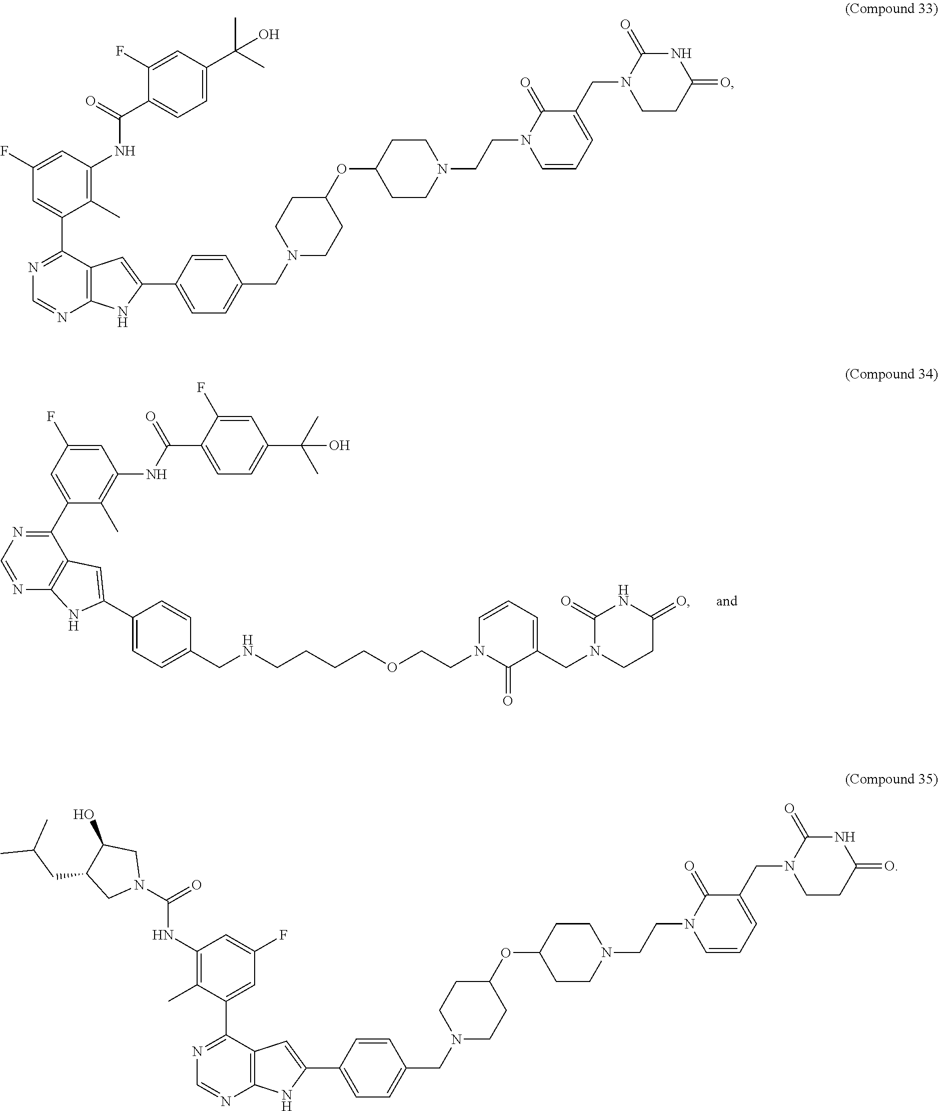 [SMILES: Cc1c(NC(=O)c2ccc(C(C)(C)O)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12 ; Cc1c(NC(=O)c2ccc(C(C)(C)O)cc2F)cc(F)cc1-c1ncnc2[nH]c(-c3ccc(CNCCCCOCCn4cccc(CN5CCC(=O)NC5=O)c4=O)cc3)cc12 ; "and" ; Cc1c(NC(=O)N2C[C@H](CC(C)C)[C@@H](O)C2)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

In an embodiment, when the compound is a compound of Formula (IIA), then the compound is not a compound selected from:
rac-N-(3-(6-(4-((9-(3-(2,4-dioxotetrahydropyrimidin-1(2H)-yl)-4-methoxybenzoyl)-7-(hydroxymethyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
(R)—N-(3-(6-(4-((9-(3-(2,4-dioxotetrahydropyrimidin-1(2H)-yl)-4-methoxybenzoyl)-7-(hydroxymethyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
(S)—N-(3-(6-(4-((9-(3-(2,4-dioxotetrahydropyrimidin-1(2H)-yl)-4-methoxybenzoyl)-7-(hydroxymethyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((9-(3-(2,4-dioxotetrahydropyrimidin-1(2H)-yl)-4-ethoxybenzoyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(2-(9-(3-(2,4-dioxotetrahydropyrimidin-1(2H)-yl)-4-methylbenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(((1-(2-(1-(3-(2,4-dioxotetrahydropyrimidin-1(2H)-yl)-4-fluorobenzoyl)piperidin-4-yl)ethyl)piperidin-4-yl)oxy)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-((1-(3-(2,4-dioxotetrahydropyrimidin-1(2H)-yl)-4-methoxybenzoyl)piperidin-4-yl)methyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-((1-(3-(2,4-dioxotetrahydropyrimidin-1(2H)-yl)-4-methoxybenzoyl)piperidin-4-yl)oxy)piperidin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((9-(3-(2,4-dioxotetrahydropyrimidin-1(2H)-yl)-4-methoxybenzoyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(2-(4-((1-(3-(2,4-dioxotetrahydropyrimidin-1(2H)-yl)-4-methylbenzoyl)piperidin-4-yl)oxy)piperidin-1-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(3-(9-(3-(2,4-dioxotetrahydropyrimidin-1(2H)-yl)-4-methoxybenzoyl)-3,9-diazaspiro[5.5]undecan-3-yl)propyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(2-(9-(3-(2,4-dioxotetrahydropyrimidin-1(2H)-yl)benzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(((1-(2-(1-(3-(2,4-dioxotetrahydropyrimidin-1(2H)-yl)-4-methoxybenzoyl)piperidin-4-yl)ethyl)piperidin-4-yl)oxy)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((9-(3-(2,4-dioxotetrahydropyrimidin-1(2H)-yl)-4-fluorobenzoyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((1-(3-(1-(3-(2,4-dioxotetrahydropyrimidin-1(2H)-yl)-4-methoxybenzoyl)piperidin-4-yl)propyl)piperidin-4-yl)oxy)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-(2-(4-(3-(2,4-dioxotetrahydropyrimidin-1(2H)-yl)-4-methoxybenzoyl)piperazin-1-yl)ethyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-(2-(1-(3-(2,4-dioxotetrahydropyrimidin-1(2H)-yl)-4-methoxybenzoyl)piperidin-4-yl)ethyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
rac-N-(3-(6-(4-((1-(2-((1-(3-(2,4-dioxotetrahydropyrimidin-1(2H)-yl)-4-methoxybenzoyl)piperidin-4-yl)oxy)-3-hydroxypropyl)piperidin-4-yl)oxy)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((1-((1-(3-(2,4-dioxotetrahydropyrimidin-1(2H)-yl)-4-methoxybenzoyl)piperidin-4-yl)methyl)piperidin-4-yl)oxy)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(2-(9-(3-(2,4-dioxotetrahydropyrimidin-1(2H)-yl)-4-methoxybenzoyl)-3,9-diazaspiro[5.5]undecan-3-yl)-2-methylpropyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(((1-(2-(4-(3-(2,4-dioxotetrahydropyrimidin-1(2H)-yl)-4-methoxybenzoyl)piperazin-1-yl)ethyl)piperidin-4-yl)oxy)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-(4-(3-(2,4-dioxotetrahydropyrimidin-1(2H)-yl)-4-methoxybenzoyl)piperazine-1-carbonyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
rac-N-(3-(6-(4-((9-(3-(2,4-dioxotetrahydropyrimidin-1(2H)-yl)-4-methoxybenzoyl)-1-(hydroxymethyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
(S)—N-(3-(6-(4-((9-(3-(2,4-dioxotetrahydropyrimidin-1(2H)-yl)-4-methoxybenzoyl)-1-(hydroxymethyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
(R)—N-(3-(6-(4-((9-(3-(2,4-dioxotetrahydropyrimidin-1(2H)-yl)-4-methoxybenzoyl)-1-(hydroxymethyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-(4-(3-(2,4-dioxotetrahydropyrimidin-1(2H)-yl)-4-methoxybenzamido)butyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((9-(3-(2,4-dioxotetrahydropyrimidin-1(2H)-yl)-4-methylbenzoyl)-3, 9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
5-(2,4-dioxotetrahydropyrimidin-1(2H)-yl)-2-fluoro-N-(5-((4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)amino)pentyl)-N,4-dimethylbenzamide,
5-(2,4-dioxotetrahydropyrimidin-1(2H)-yl)-2-fluoro-N-(5-((4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)amino)pentyl)-4-methylbenzamide,
3-(2,4-dioxotetrahydropyrimidin-1(2H)-yl)-N-(5-((4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)amino)pentyl)-N,4-dimethylbenzamide,
N-(3-(6-(4-(2-(9-(3-(2,4-dioxotetrahydropyrimidin-1(2H)-yl)-4-methoxybenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((2-(3-(2,4-dioxotetrahydropyrimidin-1(2H)-yl)-4-methoxybenzoyl)-2,7-diazaspiro[3.5]nonan-7-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-(1-(3-(2,4-dioxotetrahydropyrimidin-1(2H)-yl)-4-methoxybenzoyl)piperidine-4-carbonyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(2-(4-((1-(3-(2,4-dioxotetrahydropyrimidin-1(2H)-yl)-4-methoxybenzoyl)piperidin-4-yl)oxy)piperidin-1-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((8-(2-(3-(2,4-dioxotetrahydropyrimidin-1(2H)-yl)-4-methoxybenzamido)ethyl)-2,8-diazaspiro[4.5]decan-2-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(4-((2-(4-(3-(2,4-dioxotetrahydropyrimidin-1(2H)-yl)-4-methoxybenzoyl)piperazin-1-yl)ethyl)amino)butoxy)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-(2-(4-chloro-3-(2,4-dioxotetrahydropyrimidin-1(2H)-yl)benzamido)ethyl)-1-oxa-4,9-diazaspiro[5.5]undecan-9-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((1-(2-(1-(3-(2,4-dioxotetrahydropyrimidin-1(2H)-yl)-4-methoxybenzoyl)piperidin-4-yl)ethyl)piperidin-4-yl)oxy)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-(2-(3-(2,4-dioxotetrahydropyrimidin-1(2H)-yl)-4-methoxybenzamido)ethyl)-1-oxa-4,9-diazaspiro[5.5]undecan-9-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(((4-(4-(3-(2,4-dioxotetrahydropyrimidin-1(2H)-yl)-4-methoxybenzoyl)piperazin-1-yl)butyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((((1s,4s)-4-((1-(3-(2,4-dioxotetrahydropyrimidin-1(2H)-yl)-4-methoxybenzoyl)piperidin-4-yl)oxy)cyclohexyl)oxy)methyl)phenyl)-7H-pyrrolo[2,3-d]pyrimidin-4-yl)-5-fluoro-2-methylphenyl)-2-fluoro-4-(2-hydroxypropan-2-yl)benzamide,
4-chloro-3-(2,4-dioxotetrahydropyrimidin-1(2H)-yl)-N-(5-((4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)amino)pentyl)-N-methylbenzamide,
N-(3-(6-(4-((1-(2-(1-(3-(2,4-dioxotetrahydropyrimidin-1(2H)-yl)-4-methoxybenzoyl)piperidin-4-yl)ethyl)piperidin-4-yl)methoxy)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((9-(2-(3-(2,4-dioxotetrahydropyrimidin-1(2H)-yl)-4-methoxybenzamido)ethyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((3-((2-(4-(3-(2,4-dioxotetrahydropyrimidin-1(2H)-yl)-4-methoxybenzoyl)piperazin-1-yl)ethyl)amino)propoxy)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(((1r,4r)-4-((1-(3-(2,4-dioxotetrahydropyrimidin-1(2H)-yl)-4-methoxybenzoyl)piperidin-4-yl)oxy)cyclohexyl)oxy)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(2-(9-(3-(2,4-dioxotetrahydropyrimidin-1(2H)-yl)-4-fluorobenzoyl)-3,9-diazaspiro[5.5]undecan-3-yl)ethyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-(3-(4-chloro-3-(2,4-dioxotetrahydropyrimidin-1(2H)-yl)benzamido)propyl)-1-oxa-4,9-diazaspiro[5.5]undecan-9-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((8-(3-(2,4-dioxotetrahydropyrimidin-1(2H)-yl)-4-methoxybenzoyl)-2,8-diazaspiro[4.5]decan-2-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((4-((1-(3-(2,4-dioxotetrahydropyrimidin-1(2H)-yl)-4-methylbenzoyl)piperidin-4-yl)oxy)piperidin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((1-(2-(4-(3-(2,4-dioxotetrahydropyrimidin-1(2H)-yl)-4-methoxybenzoyl)piperazin-1-yl)ethyl)piperidin-4-yl)methoxy)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((2-(2-(3-(2,4-dioxotetrahydropyrimidin-1(2H)-yl)-4-methoxybenzamido)ethyl)-2,7-diazaspiro[3.5]nonan-7-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((2-(2-(3-(2,4-dioxotetrahydropyrimidin-1(2H)-yl)-4-methoxybenzamido)ethyl)-2,8-diazaspiro[4.5]decan-8-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-(((1s,4s)-4-((1-(3-(2,4-dioxotetrahydropyrimidin-1(2H)-yl)-4-methoxybenzoyl)piperidin-4-yl)oxy)cyclohexyl)oxy)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((2-(3-(2,4-dioxotetrahydropyrimidin-1(2H)-yl)-4-methoxybenzoyl)-2,8-diazaspiro[4.5]decan-8-yl)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((((1r,4r)-4-((1-(3-(2,4-dioxotetrahydropyrimidin-1(2H)-yl)-4-methoxybenzoyl)piperidin-4-yl)oxy)cyclohexyl)oxy)methyl)phenyl)-7H-pyrrolo[2,3-d]pyrimidin-4-yl)-5-fluoro-2-methylphenyl)-2-fluoro-4-(2-hydroxypropan-2-yl)benzamide,
N-(3-(6-(4-((9-(3-(4-chloro-3-(2,4-dioxotetrahydropyrimidin-1(2H)-yl)benzamido)propyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide, and
N-(3-(6-(4-((1-(2-((1-(3-(2,4-dioxotetrahydropyrimidin-1(2H)-yl)-4-methoxybenzoyl)piperidin-4-yl)oxy)ethyl)piperidin-4-yl)oxy)phenyl)-7H-pyrrolo[2,3-d]pyrimidin-4-yl)-5-fluoro-2-methylphenyl)-2-fluoro-4-(2-hydroxypropan-2-yl)benzamide, or a pharmaceutically acceptable salt thereof.

### Definitions

One embodiment is a compound of any of the formulae described herein, e.g., a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, that modulates, e.g., decreases the amount of a targeted protein or protein of interest, e.g., one or more proteins from Table 1 or Table 2.

Another embodiment is a compound of any of the formulae described herein, e.g., a compound of Formula (I), (II), (III), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, that degrades a targeted protein through the ubiquitin-proteasome pathway (UPP).

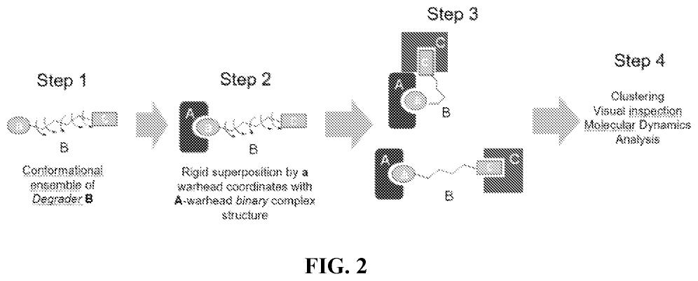

The formation of a viable ternary complex among the target protein, the bifunctional degrader, and the E3 ligase substrate receptor is enabled by the use of targeted bifunctional degraders, relying on three components, the “targeting ligand” and the “targeting ligase binder” (also termed “warheads”) and the joining segment, termed the “linker.” The likelihood that a bifunctional degrader may form an energetically favored viable complex can be assessed using an in silico computational approach. Energetic unfavorability can arise through enthalpic contributions (steric or electronic clashes between the protein targets and the degrader), entropic contributions (reduction in the degrees of freedom upon formation of the ternary complex), or a combination of the two. Using in silico methods, unfavorable linkers can be quickly identified and deprioritized. Various methods have been described for designing bifunctional degraders. See Drummond and Williams, J. Chem. Inf. Model. 59:1634-1644 (2019). The in silico ternary complex modelling protocol consists of four steps (see FIG. 2): (1) generate the conformational ensemble of the bifunctional degraders. For this task, various conformational searches methods available in standard modelling programs can be used. (2) Rigidly superimpose using the coordinates one of the warheads (either the “targeting ligand” or the “targeting ligase binder”) with the same warhead bound in the binary complex structure, as observed in crystal structure or by docking it in the respective protein. (3) Filter to retain sterically competent conformations of the bifunctional degrader with the first protein. (4) Rigidly superimpose the warhead bound in the other binary complex structure to the coordinates of the corresponding warhead in the degrader. Conformations of the targeted bifunctional degraders causing serious clashes between any of the three components of the ternary complexes are filtered out. The saved generated conformations could be further clustered and refined using standard molecular dynamics approaches. This allows the relaxation of minor steric clashes and electrostatic mismatches. It also gives an indication of the stability of the ternary complex; linkers that do not bring the proteins into contact can be said to be entropically disfavored.

For example, the method described herein has been applied to Compounds 01 and 02. Despite both compounds binding to CRBN (Table 4), Compound 2 enabled the ternary complex formation according to the method described herein, and as such degrades BTK (>95%). However, Compound 01 was predicted to not form a ternary complex and experimentally no degradation of BTK was observed (Table 4).

It is also possible to design modified linkers using de novo or generative methods to enhance physicochemical properties or some other scoring metric. See Ertl and Lewis, J. Comput Aided Mol. Des. 26(11): 1207-1215 (2012). It is possible to combine both the assessment for ternary complex formation and having favorable properties, to identify an optimal linker space.

The term “a therapeutically effective amount” of a compound described herein refers to an amount of the compound described herein that will elicit the biological or medical response of a subject, for example, reduction or inhibition of an enzyme or a protein activity, or ameliorate symptoms, alleviate conditions, slow or delay disease progression, or prevent a disease, etc. In one embodiment, the term “a therapeutically effective amount” refers to the amount of the compound described herein that, when administered to a subject, is effective to (1) at least partially alleviate, prevent and/or ameliorate a condition, or a disorder or a disease (i) mediated by a target protein, (ii) associated with activity of a target protein, or (iii) characterized by activity (normal or abnormal) of a target protein; or (2) reduce or inhibit the activity of a target protein; or (3) reduce or inhibit the expression of a target protein. These effects may be achieved for example by reducing the amount of a target protein by degrading of the target protein. In one embodiment, the term “a therapeutically effective amount” refers to the amount of the compound described herein that, when administered to a cell, or a tissue, or a non-cellular biological material, or a medium, is effective to at least partially reduce or inhibit the activity of target protein; or at least partially reduce or inhibit the expression of a target protein, for example by degrading a target protein.

As used herein, the term cancer refers to a neoplastic disease and includes for instance solid tumors, such as, e.g. sarcomas or carcinomas or blood cancer, such as, e.g. leukemia or myeloma, or cancers of lymphatic system such as lymphoma, or mixed types thereof.

As used herein, the terms “degrades”, “degrading”, or “degradation” refers to the partial or full breakdown of a target protein by the cellular proteasome system to an extent that reduces or eliminates the biological activity (especially aberrant activity) of target protein. Degradation may be achieved through mediation of an E3 ligase, in particular, E3-ligase complexes comprising the protein Cereblon. As used herein, the term “modulation of target protein activity” or “modulating target activity” means the alteration of, especially reduction, suppression or elimination, of target protein's activity. This may be achieved by degrading the target protein in vivo or in vitro. The amount of target protein degraded can be measured by comparing the amount of target protein remaining after treatment with a compound described herein as compared to the initial amount or level of target protein present as measured prior to treatment with a compound described herein. In an embodiment, at least about 30% of the target protein is degraded compared to initial levels. In an embodiment, at least about 40% of the target protein is degraded compared to initial levels. In an embodiment, at least about 50% of the target protein is degraded compared to initial levels. In an embodiment, at least about 60% of the target protein is degraded compared to initial levels. In an embodiment, at least about 70% of the target protein is degraded compared to initial levels. In an embodiment, at least about 80% of the target protein is degraded compared to initial levels. In an embodiment, at least about 90% of the target protein is degraded compared to initial levels. In an embodiment, at least about 95% of the target protein is degraded compared to initial levels. In an embodiment, over 95% of the target protein is degraded compared to initial levels. In an embodiment, at least about 99% of the target protein is degraded compared to initial levels.

In an embodiment, the target protein is degraded in an amount of from about 30% to about 99% compared to initial levels. In an embodiment, the target protein is degraded in an amount of from about 40% to about 99% compared to initial levels. In an embodiment, the target protein is degraded in an amount of from about 50% to about 99% compared to initial levels. In an embodiment, the target protein is degraded in an amount of from about 60% to about 99% compared to initial levels. In an embodiment, the target protein is degraded in an amount of from about 70% to about 99% compared to initial levels. In an embodiment, the target protein is degraded in an amount of from about 80% to about 99% compared to initial levels. In an embodiment, the target protein is degraded in an amount of from about 90% to about 99% compared to initial levels. In an embodiment, the target protein is degraded in an amount of from about 95% to about 99% compared to initial levels. In an embodiment, the target protein is degraded in an amount of from about 90% to about 95% compared to initial levels.

As used herein, the term “selectivity for the target protein” means, for example, a compound described herein degrades the target protein in preference to, or to a greater extent than, another protein or proteins.

As used herein, the term “subject” refers to an animal. Typically, the animal is a mammal. A subject also refers to, for example, primates (e.g., humans, male or female), cows, sheep, goats, horses, dogs, cats, rabbits, rats, mice, fish, birds, and the like. In an embodiment, the subject is a primate. In a preferred embodiment, the subject is a human.

As used herein, the terms “inhibit”, “inhibition”, or “inhibiting” refer to the reduction or suppression of a given condition, symptom, or disorder, or disease, or a significant decrease in the baseline activity of a biological activity or process.

As used herein, the terms “treat”, “treating”, or “treatment” of any disease or disorder refer In an embodiment, to ameliorating the disease or disorder (i.e., slowing or arresting or reducing the development of the disease or at least one of the clinical symptoms thereof). In an embodiment, “treat”, “treating”, or “treatment” refers to alleviating or ameliorating at least one physical parameter including those which may not be discernible by the patient.

As used herein, the term “preventing” refers to a reduction in the frequency of, or delay in the onset of, symptoms of the condition or disease.

As used herein, a subject is “in need of” a treatment if such subject would benefit biologically, medically, or in quality of life from such treatment.

As used herein, the term “a,” “an,” “the” and similar terms used in the context of the disclosure (especially in the context of the claims) are to be construed to cover both the singular and plural unless otherwise indicated herein or clearly contradicted by the context.

The term “alkyl” refers to a radical of a straight-chain or branched saturated hydrocarbon group having from 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tertiary amyl), and hexyl (C₆) (e.g., n-hexyl).

“Alkylene” refers to a divalent radical of an alkyl group, e.g., —CH₂—, —CH₂CH₂—, and —CH₂CH₂CH₂—.

“Heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC1 alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC1-10 alkyl. In certain embodiments, the heteroalkyl group is a substituted heteroC1-10 alkyl.

“Heteroalkylene” refers to a divalent radical of a heteroalkyl group.

“Alkoxy” or “alkoxyl” refers to an —O-alkyl radical. In some embodiments, the alkoxy groups are methoxy, ethoxy, n-propoxy, isopropoxy, n-butoxy, tert-butoxy, sec-butoxy, n-pentoxy, n-hexoxy, and 1,2-dimethylbutoxy. In some embodiments, alkoxy groups are lower alkoxy, i.e., with between 1 and 6 carbon atoms. In some embodiments, alkoxy groups have between 1 and 4 carbon atoms.

As used herein, the term “aryl” refers to a stable, aromatic, mono- or bicyclic ring radical having the specified number of ring carbon atoms. Examples of aryl groups include, but are not limited to, phenyl, 1-naphthyl, 2-naphthyl, and the like. The related term “aryl ring” likewise refers to a stable, aromatic, mono- or bicyclic ring having the specified number of ring carbon atoms.

As used herein, the term “heteroaryl” refers to a stable, aromatic, mono- or bicyclic ring radical having the specified number of ring atoms and comprising one or more heteroatoms individually selected from nitrogen, oxygen and sulfur. The heteroaryl radical may be bonded via a carbon atom or heteroatom. Examples of heteroaryl groups include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl, quinolinyl, isoquinolinyl, indolyl, indazolyl, oxadiazolyl, benzothiazolyl, quinoxalinyl, and the like. The related term “heteroaryl ring” likewise refers to a stable, aromatic, mono- or bicyclic ring having the specified number of ring atoms and comprising one or more heteroatoms individually selected from nitrogen, oxygen and sulfur.

As used herein, the term “carbocyclyl” refers to a stable, saturated or unsaturated, non-aromatic, mono- or bicyclic (fused, bridged, or spiro) ring radical having the specified number of ring carbon atoms. Examples of carbocyclyl groups include, but are not limited to, the cycloalkyl groups identified above, cyclobutenyl, cyclopentenyl, cyclohexenyl, and the like. In an embodiment, the specified number is C₃-C₁₂ carbons. The related term “carbocyclic ring” likewise refers to a stable, saturated or unsaturated, non-aromatic, mono- or bicyclic (fused, bridged, or spiro) ring having the specified number of ring carbon atoms. In an embodiment, the carbocyclyl can be substituted or unsubstituted. In an embodiment, the carbocyclyl can be substituted with 0-4 occurrences of R^{a}, wherein each R^{a} is independently selected from the group consisting of C₁₋₆ alkyl, C₁₋₆ alkoxyl, and halogen.

As used herein, the term “heterocyclyl” refers to a stable, saturated or unsaturated, non-aromatic, mono- or bicyclic (fused, bridged, or spiro) ring radical having the specified number of ring atoms and comprising one or more heteroatoms individually selected from nitrogen, oxygen and sulfur. The heterocyclyl radical may be bonded via a carbon atom or heteroatom. In an embodiment, the specified number is C₃-C₁₂ carbons. Examples of heterocyclyl groups include, but are not limited to, azetidinyl, oxetanyl, pyrrolinyl, pyrrolidinyl, tetrahydrofuryl, tetrahydrothienyl, piperidyl, piperazinyl, tetrahydropyranyl, morpholinyl, perhydroazepinyl, tetrahydropyridinyl, tetrahydroazepinyl, octahydropyrrolopyrrolyl, and the like. The related term “heterocyclic ring” likewise refers to a stable, saturated or unsaturated, non-aromatic, mono- or bicyclic (fused, bridged, or spiro) ring having the specified number of ring atoms and comprising one or more heteroatoms individually selected from nitrogen, oxygen and sulfur. In an embodiment, the heterocyclyl can be substituted or unsubstituted. In an embodiment, the heterocyclyl can be substituted with 0-4 occurrences of R^{a}, wherein each R^{a} is independently selected from the group consisting of C₁₋₆ alkyl, C₁₋₆ alkoxyl, and halogen.

As used herein, “spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. For example, a (C₃-C₁₂)spirocycloalkyl is a spirocycle containing between 3 and 12 carbon atoms.

As used herein, “spiroheterocycloalkyl” or “spiroheterocyclyl” means a spirocycle wherein at least one of the rings is a heterocycle wherein one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

As used herein, “halo” or “halogen” refers to fluorine (fluoro, —F), chlorine (chloro, —Cl), bromine (bromo, —Br), or iodine (iodo, —I).

As used herein, “haloalkyl” means an alkyl group substituted with one or more halogens. Examples of haloalkyl groups include, but are not limited to, trifluoromethyl, difluoromethyl, pentafluoroethyl, and trichloromethyl.

As used herein, “substituted”, whether preceded by the term “optionally” or not, means that one or more hydrogens of the designated moiety are replaced with a suitable substituent.

As used herein, the definition of each expression, e.g., alkyl, m, n, etc., when it occurs more than once in any structure, is intended to be independent of its definition elsewhere in the same structure.

Various embodiments of the disclosure are described herein. It will be recognized that features specified in each embodiment may be combined with other specified features, including as indicated in the embodiments below, to provide further embodiments of the present disclosure.

It is understood that in the following embodiments, combinations of substituents or variables of the depicted formulae are permissible only if such combinations result in stable compounds.

Definitions of specific functional groups and chemical terms are described in more detail below. The chemical elements are identified in accordance with the Periodic Table of the Elements, CAS version, Handbook of Chemistry and Physics, 75^{th} ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} ed, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} ed, Cambridge University Press, Cambridge, 1987.

Certain compounds described herein may exist in particular geometric or stereoisomeric forms. If, for instance, a particular enantiomer of a compound described herein is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Unless otherwise stated, structures depicted herein are also meant to include geometric (or conformational) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the disclosed compounds are within the scope of the disclosure. Unless otherwise stated, all tautomeric forms of the compounds described herein are within the scope of the disclosure. Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C or ¹⁴C enriched carbon are within the scope of this disclosure. Such compounds are useful, for example, as analytical tools, as probes in biological assays, or as therapeutic agents in accordance with the disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer. ee=(90−10)/100×100=80%.

Thus, a composition containing 90% of one enantiomer and 10% of the other enantiomer is said to have an enantiomeric excess of 80%. The compounds or compositions described herein may contain an enantiomeric excess of at least 50%, 75%, 90%, 95%, or 99% of one form of the compound, e.g., the S-enantiomer. In other words such compounds or compositions contain an enantiomeric excess of the S enantiomer over the R enantiomer.

Where a particular enantiomer is preferred, it may, in some embodiments be provided substantially free of the corresponding enantiomer, and may also be referred to as “optically enriched.” “Optically enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See e.g., Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw Hill, N Y, 1962); Wilen, S. H. Tables of Resolving Agents and Optical Resolutions p. 268 (E. L. Eliel, Ed., Univ. of Notre Dame Press, Notre Dame, Ind. 1972).

All methods described herein can be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the disclosure otherwise claimed.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds described herein into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

### Pharmaceutically Acceptable Salts

Pharmaceutically acceptable salts of the compounds described herein are also contemplated for the uses described herein. As used herein, the terms “salt” or “salts” refer to an acid addition or base addition salt of a compound described herein. “Salts” include in particular “pharmaceutical acceptable salts.” The term “pharmaceutically acceptable salts” refers to salts that retain the biological effectiveness and properties of the compounds disclosed herein and, which typically are not biologically or otherwise undesirable. In many cases, the compounds disclosed herein are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium, and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine.

Another embodiment is a compound of Formula (I), (II), (IIA), (BF-I), (BF-II), (BF-III), (BF-IV) (BF-V-A), (BF-V-B), or Compounds 1-35 as an acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate, or xinafoate salt form.

### Pharmaceutical Compositions

Another embodiment is a pharmaceutical composition comprising one or more compounds described herein or a pharmaceutically acceptable salt, hydrate, solvate, prodrug, stereoisomer, or tautomer thereof, and one or more pharmaceutically acceptable carrier(s). The term “pharmaceutically acceptable carrier” refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions of the disclosure are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween®, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax, and polyethylene glycols.

The pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

The pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The amount of the compounds of the present disclosure that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

### Isotopically Labelled Compounds

A compound described herein or a pharmaceutically acceptable salt, hydrate, solvate, prodrug, stereoisomer, or tautomer thereof, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorous, fluorine, and chlorine, such as ²H, 3H, ¹¹C, ¹³C, ¹⁴C, ¹⁵N, ¹⁸F, ³¹P, ³²P, ³⁵S, ³⁶Cl, ¹²³I, ¹²⁴, ¹²⁵I, respectively. The disclosure includes various isotopically labeled compounds as defined herein, for example, those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds described herein or a pharmaceutically acceptable salt, hydrate, solvate, prodrug, stereoisomer, or tautomer thereof, can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound described herein or a pharmaceutically acceptable salt, hydrate, solvate, prodrug, stereoisomer, or tautomer thereof. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound described herein is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Dosages

Toxicity and therapeutic efficacy of compounds described herein, including pharmaceutically acceptable salts and deuterated variants, can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and thereby reduce side effects.

Data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### Methods of Use

Another embodiment is a method of modulating a Target Protein, e.g., a Target Protein listed in Table 1 or Table 2, in a subject in need thereof, the method comprising administering to the subject a therapeutically effective amount of a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof.

Another embodiment is a method of inhibiting a Target Protein, e.g., a Target Protein listed in Table 1 or Table 2, in a subject in need thereof, the method comprising administering to the subject a therapeutically effective amount of a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof.

Another embodiment is a method for inducing degradation of a Target Protein, e.g., a Target Protein listed in Table 1 or Table 2, in a subject in need thereof, the method comprising administering to the subject a therapeutically effective amount of a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof.

In another aspect, the disclosure provides a method of inhibiting, reducing, or eliminating the activity of a Target Protein, e.g., a Target Protein listed in Table 1 or Table 2, the method comprising administering to the subject a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof.

In an embodiment, inhibiting, reducing, or eliminating the activity of a Target Protein, e.g., a Target Protein listed in Table 1 or Table 2, comprises recruiting a ligase (e.g., Cereblon E3 Ubiquitin ligase) with the Targeting Ligase Binder, e.g., a Targeting Ligase Binder described herein, of the compound, e.g., a compound of Formula (I), (II), (IIA), (BF-I), (BF-II), (BF-III), (BF-IV) (BF-V-A), (BF-V-B), or Compounds 1-35, forming a ternary complex of the Target Protein, the compound, and the ligase, to thereby inhibit, reduce or eliminate the activity of the Target Protein.

Another embodiment is a method of treating or preventing a respiratory disorder, a proliferative disorder, an autoimmune disorder, an autoinflammatory disorder, an inflammatory disorder, a neurological disorder, and an infectious disease or disorder mediated by a Target Protein, e.g., a Target Protein listed in Table 1 or Table 2, in a subject in need thereof, the method comprising administering to the subject a therapeutically effective amount of a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof.

Another embodiment is a method of treating or preventing a cancer in a subject in need thereof, the method comprising administering to the subject a therapeutically effective amount of a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof.

In an embodiment, the cancer is a neoplastic disease and includes, for instance, solid tumors such as e.g. sarcomas or carcinomas or blood cancer such as e.g. leukemia or myeloma, or cancers of lymphatic system such as lymphoma, or mixed types thereof.

In another aspect, the disclosure provides compounds of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, for use in inhibiting or modulating a target protein in a subject in need thereof.

In another aspect, the disclosure provides compounds of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, for use in inhibiting a target protein in a subject in need thereof.

Another embodiment is a pharmaceutical composition comprising a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, and a pharmaceutically acceptable carrier, for use in inhibiting a Target Protein, e.g., a Target Protein listed in Table 1 or Table 2, in a subject in need thereof.

Another embodiment is compounds of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, for use in treating or preventing a respiratory disorder, a proliferative disorder, an autoimmune disorder, an autoinflammatory disorder, an inflammatory disorder, a neurological disorder, and an infectious disease or disorder mediated by a Target Protein, e.g., a Target Protein listed in Table 1 or Table 2, in a subject in need thereof.

Another embodiment is compounds of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, for use in treating or preventing a cancer in a subject in need thereof.

In an embodiment, the cancer is a neoplastic disease and includes, for instance, solid tumors such as e.g. sarcomas or carcinomas or blood cancer such as e.g. leukemia or myeloma, or cancers of lymphatic system such as lymphoma, or mixed types thereof.

Another embodiment is the use of a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, in the manufacture of a medicament for inhibiting or modulating a Target Protein, e.g., a Target Protein listed in Table 1 or Table 2, in a subject in need thereof.

Another embodiment is a use of a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, in the manufacture of a medicament for inhibiting a Target Protein, e.g., a Target Protein listed in Table 1 or Table 2, in a subject in need thereof.

Another embodiment is a use of a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, in the manufacture of a medicament for treating or preventing a cancer mediated by a Target Protein, e.g., a Target Protein listed in Table 1 or Table 2, in a subject in need thereof.

In an embodiment, the cancer is a neoplastic disease and includes, for instance, solid tumors such as e.g. sarcomas or carcinomas or blood cancer such as e.g. leukemia or myeloma, or cancers of lymphatic system such as lymphoma, or mixed types thereof.

Another embodiment is a method for treating or preventing a cancer mediated by a Target Protein, e.g., a Target Protein listed in Table 1 or Table 2, in a subject in need thereof comprising administering a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof to the subject.

In an embodiment, the cancer is a neoplastic disease and includes, for instance, solid tumors such as e.g. sarcomas or carcinomas or blood cancer such as e.g. leukemia or myeloma, or cancers of lymphatic system such as lymphoma, or mixed types thereof.

Another embodiment is a use of a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, in the manufacture of a medicament for treating or preventing a respiratory disorder, a proliferative disorder, an autoimmune disorder, an autoinflammatory disorder, an inflammatory disorder, a neurological disorder, and an infectious disease or disorder in a subject in need thereof.

### Combination Therapy

Another embodiment is a pharmaceutical combination comprising a compound of Formula (I), (II), (IIA), (BF-I), (BF-II), (BF-III), (BF-IV) (BF-V-A), (BF-V-B), or Compounds 1-35, or a pharmaceutically acceptable salt, hydrate, solvate, prodrug, stereoisomer, or tautomer thereof, and one or more additional therapeutic agent(s) for simultaneous, separate or sequential use in therapy.

In an embodiment, the additional therapeutic agent is selected from the group consisting of: an antiproliferative agent, anticancer agent, immunomodulatory agent, an anti-inflammatory agent, a neurological treatment agent, an anti-viral agent, an anti-fungal agent, anti-parasitic agent, an antibiotic, and a general anti-infective agent.

In an embodiment, the additional therapeutic agent is selected from the group consisting of: a second a target protein inhibitor.

### Methods of Making

The compounds described herein can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described below.

The disclosed compounds may be synthesized according to the general methods described in the following synthetic schemes 1, 1a, 1b, 2-4, 4a, 5, 5a, 6, 6a, 7-16, 16a, 17-18, 18a, 18b, 19, 19a, and 20-21. Starting materials are either commercially available or made by known procedures in the reported literature or as illustrated.

Compounds of formula (I) containing a linker of formula (L-I) wherein X¹ is a nitrogen-containing heterocyclyl, e.g., a piperidinyl or piperazinyl and X², L¹, L², L³ are as previously defined may be synthesized from a compound of formula (III) and a compound of formula (IV) according to Scheme 1 using a reductive amination reaction. If X¹ is a bond, then scheme 1 also provides for compounds of formula (IV) wherein L² is a primary or secondary amine to react with a compound of formula (III) to produce (I). L^{1a} is defined as a linker that is shorter by a single methylene group than L¹, wherein the formula of L¹ allows (e.g., in an embodiment where L¹ is —CH₂CH₂—, then L^{1a} is —CH₂—). Suitable L¹ include C₁₋₆ alkylene and C₁₋₆ heteroalkylene. Conditions such as ZnCl₂ and NaBH₃CN, in a solvent mixture such as THF/DMSO and MeOH may be employed. Alternative conditions include treatment with NaOAc, AcOH, and NaBH(OAc)₃ in DCM.

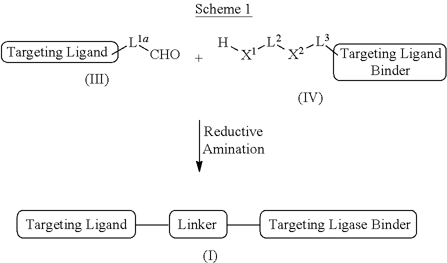

By analogy, in further embodiments, bifunctional compounds of Formulae (BF-I), (BF-II), (BF-III), (BF-V-A), (BF-V-B), and (BF-IV) wherein X¹ is a nitrogen-containing heterocyclyl, e.g., a piperidinyl or piperazinyl and R^{d1}, R^{d2}, R^{d3}, R^{d4}, R^{d5}, R^{d6}, R^{d7}, R^{d8}, X², L¹, L², L³, m and n are as previously defined, may be made from a compound of formula (III) and compounds of formula (IVa), (IVb), (IVc), (IVd), (IVe), and (IVf), respectively, according to Scheme 1a.

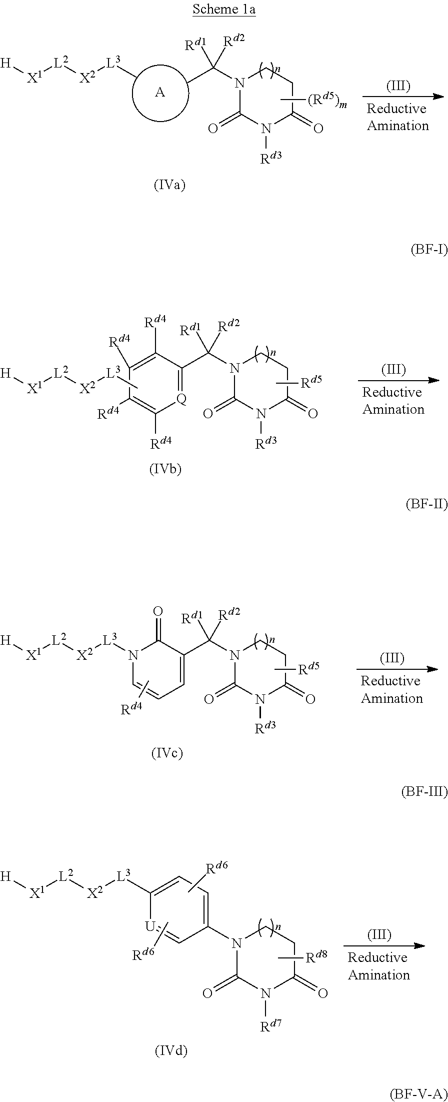

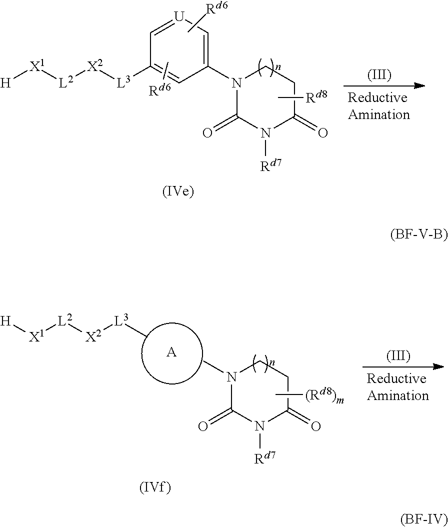

In a further embodiment, a compound of formula (II) wherein X¹ is a nitrogen-containing heterocyclyl, e.g., a piperidinyl or piperazinyl and R^{d1}, R^{d2}, R^{d3}, R^{d4}, R^{d5}, R^{d6}, R^{d7}, R^{d8}, X², L¹, L², L³, m and n are as previously defined, may be made from a compound of formula (IIIa), wherein R^{1a}, R^{2a}, R^{3a}, R^{4a}, R^{5a} and Lia are as previously defined and a compound of formula (IVc) according to Scheme 1b. Conditions similar to those described herein above apply.

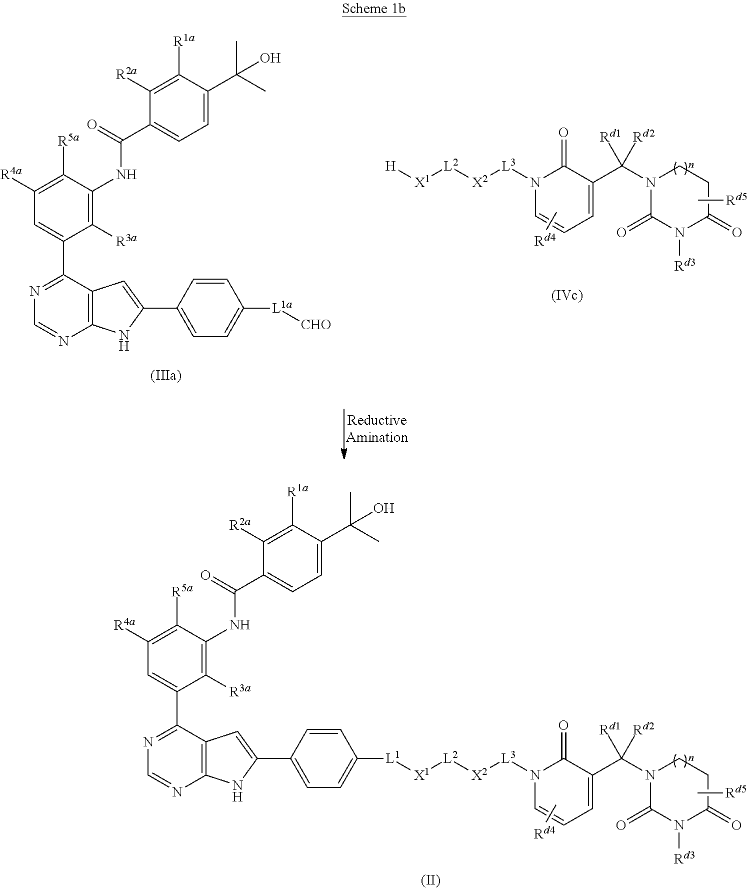

The intermediates of formulae (IVa), (IVb), (IVc), (IVd), (IVe) and (IVf) wherein X¹ is a nitrogen-containing heterocyclyl, e.g., a piperidinyl or piperazinyl, may also be applied to the synthesis of compounds of formulae (BF-I), (BF-II), (BF-III), (BF-V-A), (BF-V-B), and (BF-IV), respectively, which contain an amide linkage by reaction with a carboxylic acid of formula (V) using an amide coupling reaction according to Scheme 2. Similarly if X¹ is a bond, then scheme 2 also provides for compounds of formula (IV a-f) wherein L² is a primary or secondary amine to react with a compound of formula (III) to produce (BF-I), (BF-II), (BF-III), (BF-V-A), (BF-V-B), and (BF-IV), respectively. For compounds of formula (V), L^{1b} is defined as the subset of linkers L¹, that contain a carbonyl group and so are able to provide for compounds (V) containing a carboxylic acid functional group. Conditions include using an amide coupling reagent such as HATU, in a solvent such as DMF, in the presence of a base such as DIPEA.

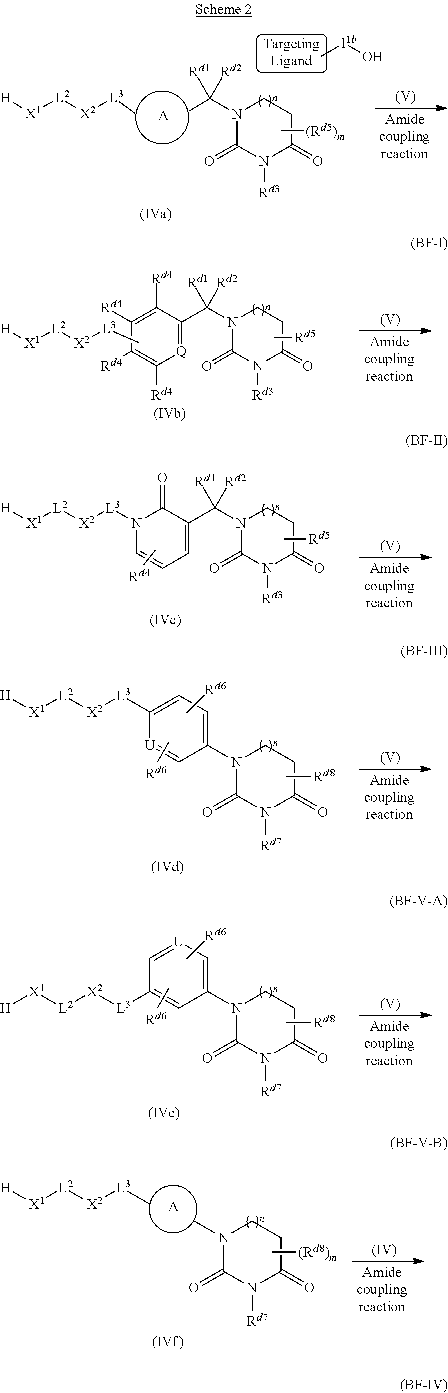

In further embodiments, bifunctional compounds of formula (BF-I), (BF-II), (BF-III), (BF-V-A), (BF-V-B), and (BF-IV) wherein X¹ is a nitrogen-containing heterocyclyl, e.g., a piperidinyl or piperazinyl may be made from compounds of formula (VI), wherein LG represents a leaving group such as a halide or a mesylate, and compounds of formula (IVa), (IVb), (IVc), (IVd), (IVe), and (IVf), respectively using an alkylation reaction according to Scheme 3. Similarly if X¹ is a bond, then scheme 3 also provides for compounds of formula (IV a-f) wherein L² is a primary or secondary amine to react with a compound of formula (VI) to produce (BF-I), (BF-II), (BF-III), (BF-V-A), (BF-V-B), and (BF-IV) respectively.

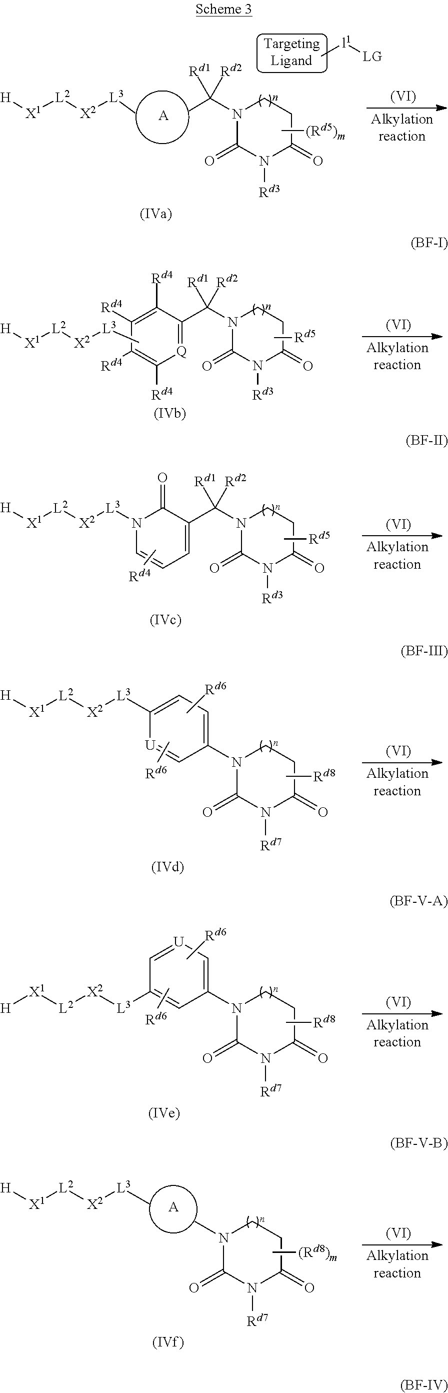

Typical conditions would be to treat an alkyl chloride of formula (VI) with an iodinating reagent such as potassium iodide and a base such as DIPEA with the appropriate amine (IVa-f) in a solvent such as DMA at a temperature such as 80° C.

Compounds of formula (BF-I), (BF-II), (BF-III), (BF-V-A), (BF-V-B), and (BF-IV) wherein X² is a nitrogen-containing heterocyclyl, e.g., a piperidinyl or piperazinyl may be made by reacting a compound of formula (VII) with compounds of formula (VIIIa), (VIIIb), (VIIIc), (VIIId), (VIIIe), and (VIIIf), respectively, in an amide coupling reaction according to Scheme 4. Similarly if X² is a bond, then scheme 4 also provides for compounds of formula (VIII a-f) wherein L² is a primary or secondary amine to react with a compound of formula (VII) to produce (BF-I), (BF-II), (BF-III), (BF-V-A), (BF-V-B), and (BF-IV), respectively.

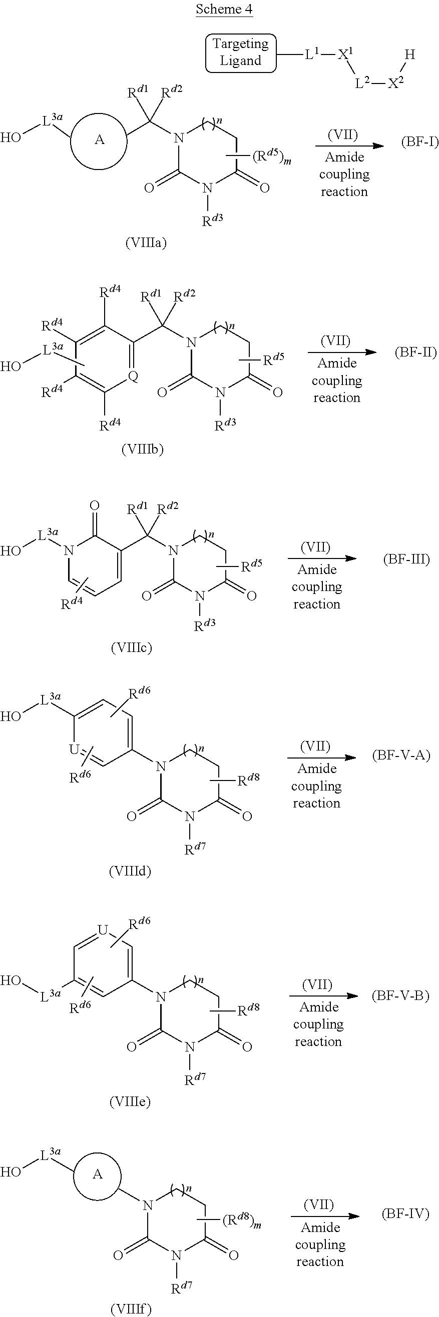

In this embodiment, L^{3a} in compound (VIIIa-f) is defined as the subset of linkers L³ that contain a carbonyl group and so are able to provide for compounds (VIIIa-f) containing a carboxylic acid functionality (e.g., in an embodiment wherein L^{3a} is —CH₂—C(O)—, then L^{3a}-OH is defined as —CH₂—CO₂H). Suitable conditions include those for amide coupling reactions as already described herein above.

Further, more specific embodiments of carboxylic acid intermediates include compounds of formula (VIIIg) and (VIIIh), which can react with a compound of formula (VII) (in a similar fashion to that described herein above for compounds (VIIIa-f)), to provide compounds of formula (BF-V-A) or (BF-V-B), according to Scheme 4a.

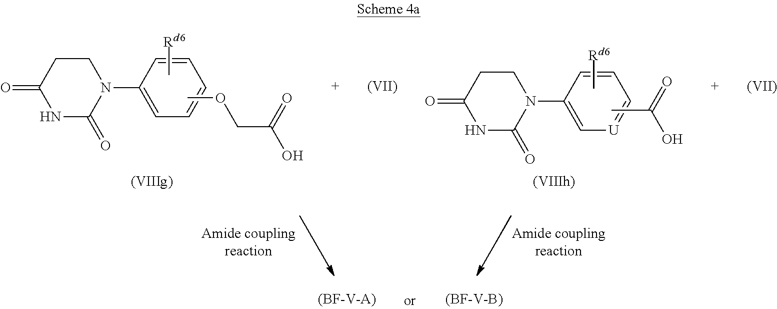

Compounds of formula (BF-I), (BF-II), (BF-III), (BF-V-A), (BF-V-B), and (BF-IV) wherein X² is a nitrogen-containing heterocyclyl, e.g., a piperidinyl or piperazinyl may also be made by reacting a compound of formula (VII) with compounds of formula (IXa), (IXb), (IXc), (IXd), (IXe), and (IXf), respectively in a reductive amination reaction according to Scheme 5. Similarly if X² is a bond, then scheme 5 also provides for compounds of formula (IXa-f) wherein L² is a primary or secondary amine to react with a compound of formula (VII) to produce (BF-I), (BF-II), (BF-III), (BF-V-A), (BF-V-B), and (BF-IV), respectively.

For compounds (IXa-f), L³b is defined as a linker that is shorter by a single methylene group than L³, wherein the formula of L³ allows (e.g., in an embodiment where L³ is —CH₂CH₂—, then L^{3b} is —CH₂—). Suitable L³ include C₁₋₆ alkylene and C₁₋₆ heteroalkylene. Conditions such as ZnCl₂ and NaBH₃CN, in a solvent mixture such as THF/DMSO and MeOH may be employed. Alternative conditions include treatment with NaOAc, AcOH, and NaBH(OAc)₃ in DCM.

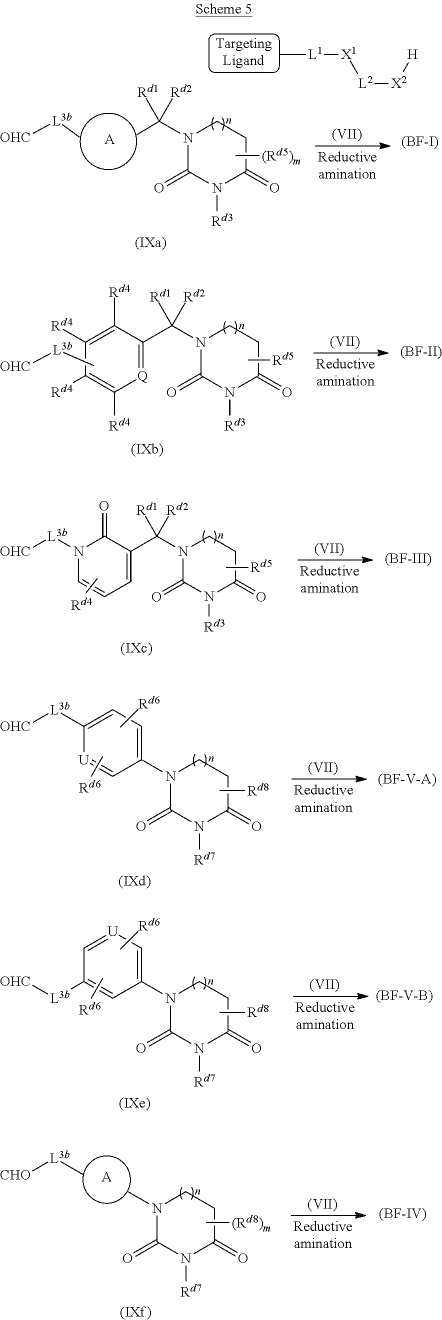

It will be apparent to those skilled in the art that masked aldehyde equivalents such as the corresponding adducts (e.g., compounds of formula (X)) formed from an aldehyde such as (IXg) and sodium metabisulfite in aqueous ethanol are equally suitable reactants for effecting this overall reductive amination transformation, the embodiment depicted in Scheme 5a being a representative example. In this case, reaction of (X) with (VII) can occur in the presence of sodium acetate and picoline borane complex in a solvent such as methanol.

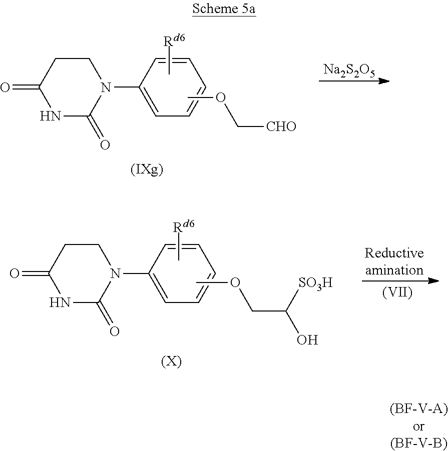

Compounds of formula (IV), more specifically compounds of formulae (IVa), (IVb), (IVc), (IVd), (IVe), and (IVf) such as those wherein both X¹ and X² are nitrogen-containing heterocyclyls, e.g., piperidinyl or piperazinyl or X¹-L²-X² is a spiroheterocyclyl, may be synthesized from the corresponding aldehydes (IXa), (IXb), (IXc), (IXd), (IXe), and (IXf), respectively according to Scheme 6. The aldehydes undergo a reductive amination reaction under conditions already described herein above using a compound of formula (XI), where PG represents a protecting group, such as t-butoxycarbonyl. A subsequent deprotection reaction using conditions such as TFA in DCM or HCl in 1,4-dioxane and methanol provides the compound of formula (IVa-f). Scheme 6 illustrates the transformation of (IXa) into (IVa) as a representative embodiment.

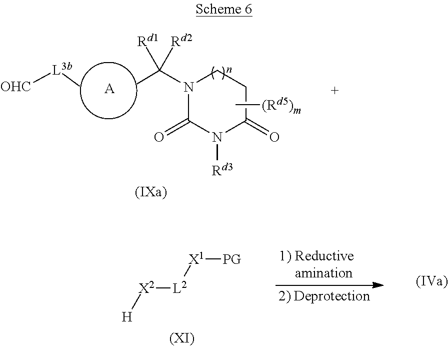

In an embodiment, a compound of formula (IXc) can undergo an analogous reductive amination with a specific example of (XI), such as (XIa), followed by deprotection under conditions already described herein above to provide a compound of formula (IVc-1) according to Scheme 6a. This compound (IVc-1) may then react in the same manner as other embodiments of (IVc) with a compound of formula (IIIa) in a reductive amination reaction to provide a compound of formula (II).

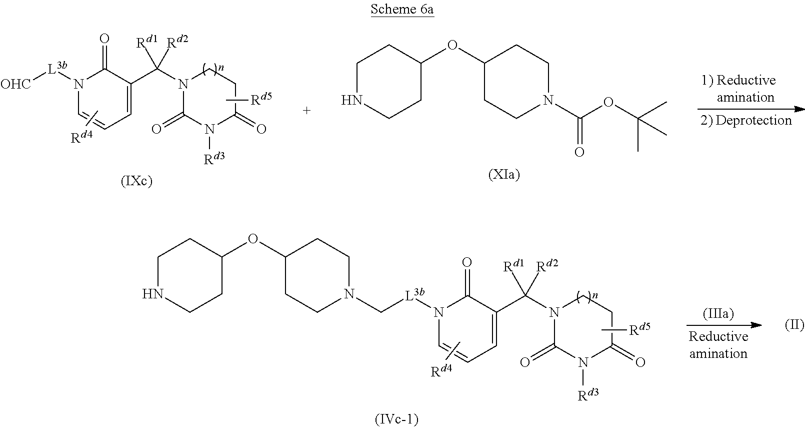

Compounds of formula (IV), and specifically compounds of formulae (IVa), (IVb), (IVc), (IVd), (IVe), and (IVf) such as those wherein both X¹ and X² are nitrogen-containing heterocyclyls, e.g., piperidinyl or piperazinyl or X¹-L²-X² is a spiroheterocyclyl, may also be synthesized from the corresponding carboxylic acids (VIIIa), (VIIIb), (VIIIc), (VIIId), (VIIIe), and (VIIIf), respectively according to Scheme 7. In this embodiment, an amide coupling reaction is employed with a compound of formula (XI), using a reagent such as HATU, in a solvent such as DMF, in the presence of a base such as DIPEA, followed by a deprotection reaction using conditions such as TFA in DCM or HCl in 1,4-dioxane and methanol to provide the compound of formula (IVa-f). The scheme illustrates the transformation of (VIIIa) into (IVa) as a representative embodiment.

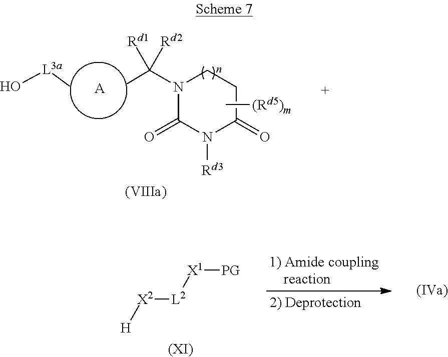

In an embodiment, compounds of formula (IV), for example a compound of formula (IVd) or (IVe) may be synthesized from a carboxylic acid of formula (VIIIg) or (VIIIh) by reacting with a monoprotected diamine (such as compound (XIII)) in an amide coupling reaction followed by a deprotection reaction using conditions as already described herein above (Scheme 8). In the examples depicted, X² is absent.

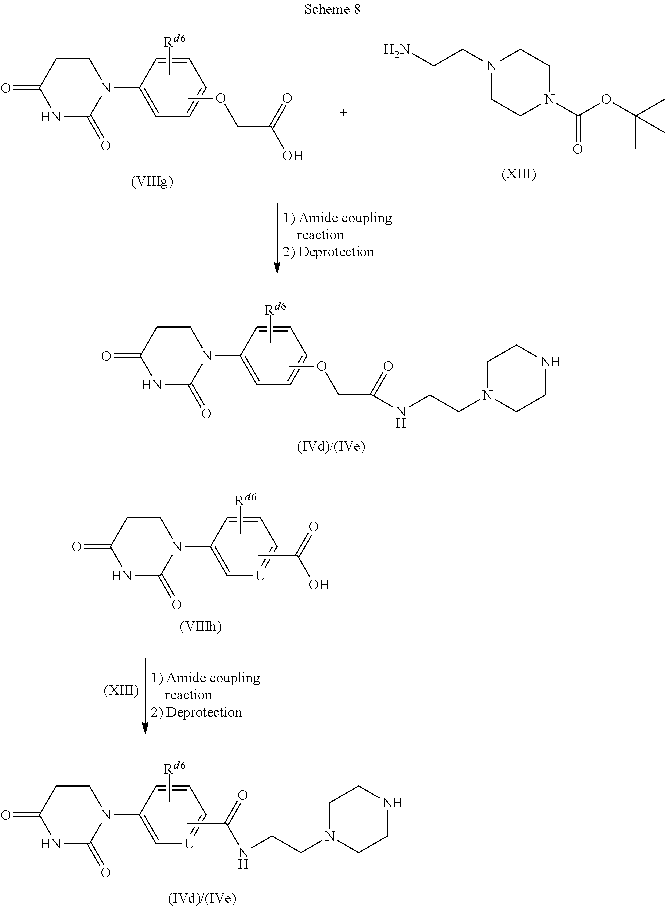

Other compounds of formula (IV), for example compounds of formula (IVd) and (IVe) wherein L³ is a C₂₋₆ alkynylene, may be synthesized according to Scheme 9. Thus, a palladium-catalyzed coupling between an alkyne compound of formula (XIV) wherein PG is a protecting group such as a t-butoxycarbonyl and a compound of formula (XV), wherein Hal is a halogen atom such as iodine, followed by a deprotection reaction afford the compound of formula (IVd) or (IVe). The palladium catalyzed reaction is a Sonogashira reaction carried out using a catalyst such as PdCl₂(PPh₃)₂ and CuI and a base, such as triethylamine in a solvent such as DMF. Optionally, the product from the palladium-catalyzed reaction can be reduced under hydrogenation conditions, using for example H₂ gas and a Pd/C catalyst, prior to the deprotection reaction. In this case, the final products (IVd/IVe) with L³ being C₁₋₆-alkylene are produced. Compounds (XIVa) and (XIVb) are specific embodiments of compound (XIV) which can undergo these reaction sequences. Compounds (XIVa) and (XIVb) may in turn be synthesized for example by an alkylation reaction of a compound of formula (XI) using an alkynylene bromide such as 4-bromo-1-butyne or propargyl bromide respectively in the presence of a base such as K₂CO₃ in a solvent such as acetonitrile.

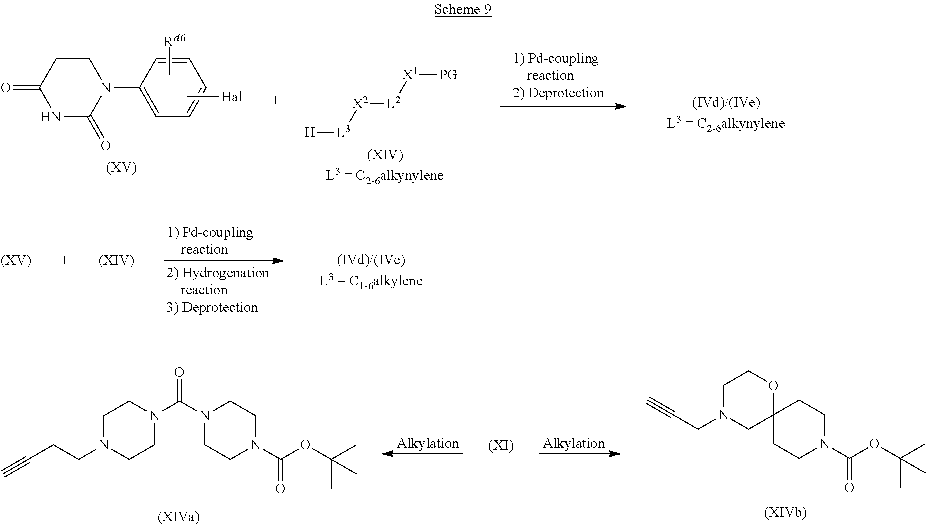

Other compounds of formula (IV), for example compounds of formula (IVd) and (IVe) wherein L³ contains an ether link, may be synthesized according to Scheme 10 starting from a phenol of formula (XVI). Thus the alkylation of (XVI) using an alkyl bromide such as (XVII) with a base such as K₂CO₃ in a solvent such as DMF provides after deprotection a compound of formula (IVd/IVe); the particular example depicted represents a compound of formula (IVd/IVe) wherein both X¹ and X² are a bond and L² is NR′. Thus, this product is able to undergo a reductive amination with a compound of formula (III) under conditions already described herein above to provide a compound of formula (I). An alternative synthetic route is to react phenol (XVI) with a N-protected amino alcohol in a Mitsunobu reaction in the presence of a phosphine reagent such as triphenylphosphine and an azo carboxylate ester such as diethylazodicarboxylate to form the ether bond, followed by a deprotection reaction to provide the compound of formula (IVd/IVe). The linker may also be built up in a sequence of steps to convert a compound of formula (XVI) into a compound of formula (IV), such as (IVg) or (IVh). In an embodiment, phenol (XVI) may react with an N-protected amino alcohol such as (XVIIIa) in a Mitsunobu reaction in the presence of a phosphine reagent such as triphenylphosphine and an azo carboxylate ester such as diethylazodicarboxylate to form an ether bond, followed by a deprotection reaction to provide a compound of formula (IVg). This compound can be extended, by a further reductive amination with a N-protected amino aldehyde such as t-butyl 4-(2-oxoethyl)piperazine-1-carboxylate to provide a chain extended compound of formula (IVh). Both (IVg) and (IVh) can react with a compound of formula (III) to provide a compound of formula (I) using a reductive amination using conditions already described herein above. In an embodiment, reductive amination with an aldehyde-ester such as t-butyl-5-oxopentanoate, followed by deprotection of the ester functionality using an acid such as TFA in DCM can give a carboxylic acid of formula (XII). Compound (XII) can react via an amide coupling under conditions described herein above, with a targeting ligand containing an available primary or secondary amine function (XXIV) to provide a compound of formula (I) wherein X¹ is a bond and L¹ is C(O).

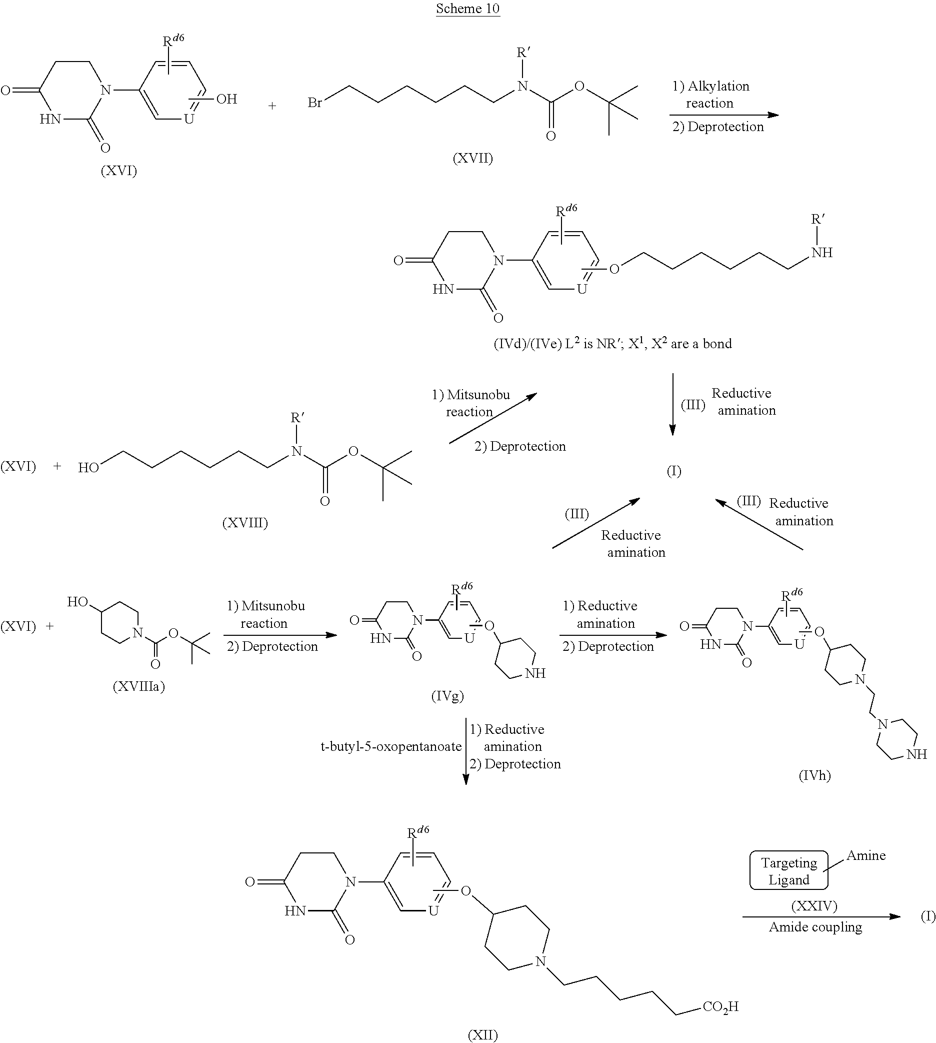

A compound of formula (IV), such as (IVd) or (IVe), wherein L³ and X¹ each represent a bond and X² is a 1,2,3-triazole can be made according to Scheme 11 using a Cu-catalyzed cycloaddition reaction between an alkyne of formula (XIX) and an azide of formula (XX) using a Cu(II) salt such as Cu(II)SO₄ and sodium L-ascorbate, in a solvent mixture such as THF and water. Deprotection of the protecting group under conditions already described herein above lead to the compound of formula (IV).

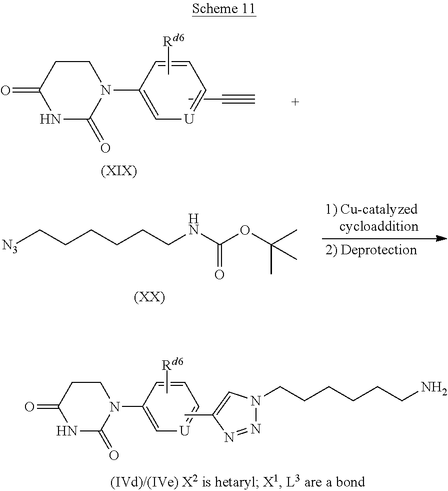

A compound of formula (VII) wherein both X¹ and X² are nitrogen-containing heterocyclyls, e.g., piperidinyl or piperazinyl or X¹-L²-X² is a spiroheterocyclyl, may be synthesized according to Scheme 12 from a compound of formula (III) and a compound of formula (XXI) following a reductive amination, deprotection sequence under conditions already described herein above. Alternatively, different compounds of formula (VII) can be prepared from carboxylic acids of formula (V), by reacting with a compound of formula (XXI) firstly in an amide coupling reaction, followed by a deprotection reaction under conditions already described herein above. This scheme also provides for compounds of certain cases of formula (VII) wherein certain linker elements are a bond, one example being when using the compound (XXIa) wherein both X¹ and X² are a bond.

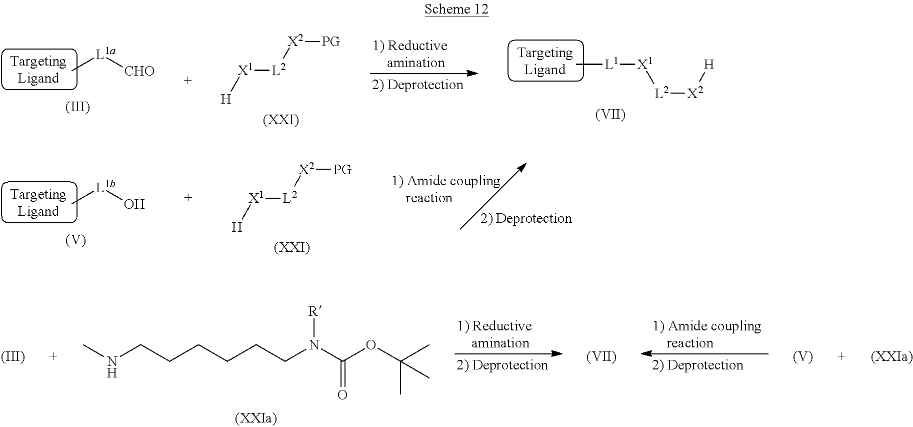

Compounds of formula (III) may also be converted to primary amines of formula (XXII) using a reductive amination using, for example, methanolic ammonia and hydrogen gas in the presence of a catalyst, such as Raney Nickel. In a specific embodiment, a compound of formula (IIIa) reacts under similar conditions to provide (XXIIa). Subsequent reductive amination with an aldehyde of formula (XXIII) using conditions such as ZnCl₂ and NaBH₃CN, in a solvent mixture, such as THF/DMSO and MeOH, provides an example compound of Formula (II), wherein X¹ and X² are each represented by a bond (Scheme 13).

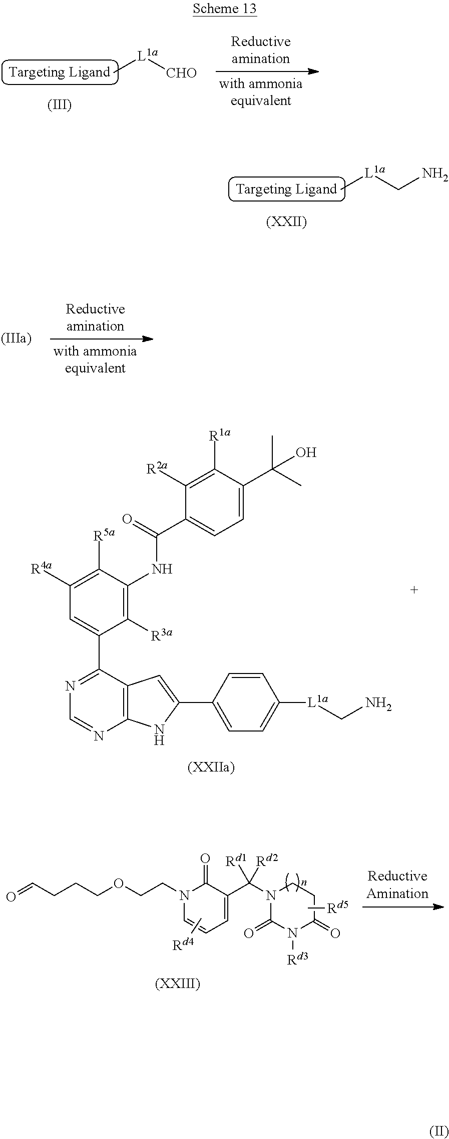

Nitriles of formula (XXV) may be reduced to amines of formula (XXVI) using conditions such as hydrogen gas and a catalyst such as Raney Nickel in the presence of aqueous ammonium hydroxide with a co-solvent such as MeOH, according to Scheme 14. These amines may react with N-protected amino acids, where in PG represents a protecting group such as a t-butoxycarbonyl group, in an amide coupling reaction, A subsequent deprotection reaction under acidic conditions provides compounds of formula (IV); in an embodiment (XXVI) may react with (XXVII) to provide the compounds of formula (IVi) wherein both X¹ and X² are a bond.

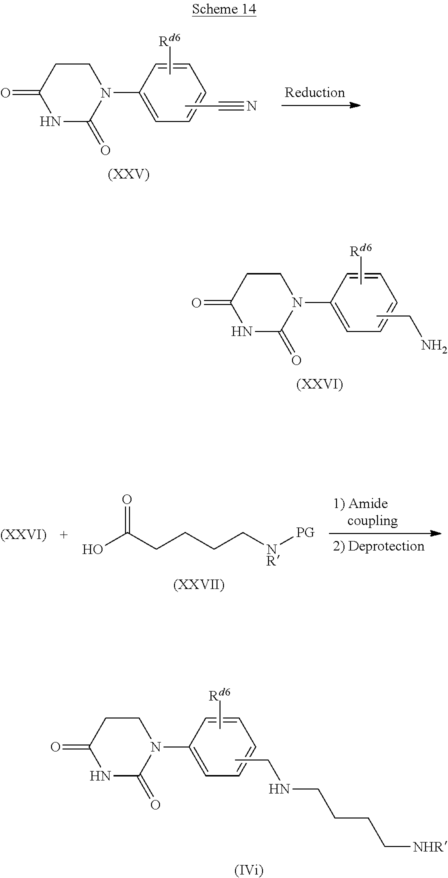

A Mitsunobu coupling can be used to synthesize compounds of formula (VII) wherein the linker contains an ether linkage directly to the targeting ligand, from a compound of formula (XXVIII), wherein the hydroxy group is part of a phenol or a hydroxypyridine, followed by a deprotection reaction, according to Scheme 15.

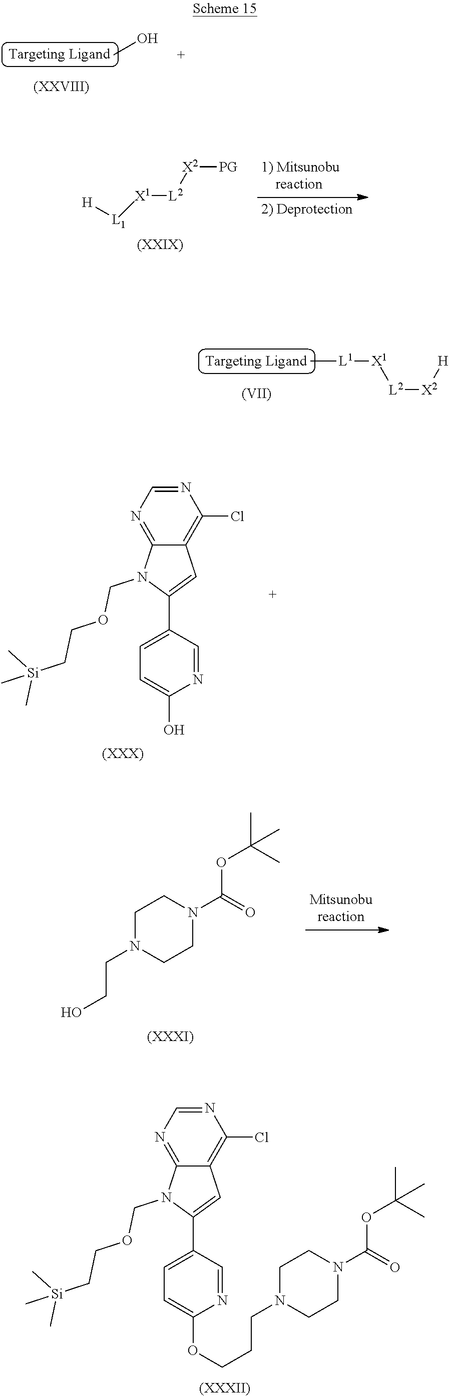

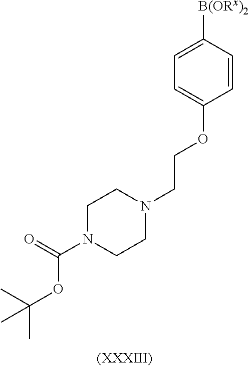

To those skilled in the art of organic synthesis, it will be understood that the molecules of the invention may be built up in a modular way which allows for different reaction orders. For example, the Mitsunobu coupling described in Scheme 15 may be applied to a synthesis fragment such as compound (XXX) wherein the pyridyl ring is part of the targeting ligand. Thus (XXX) can undergo reaction with the compound of formula (XXXI) to provide another reaction intermediate (XXXII). This intermediate (XXXII) then requires further synthetic procedures to construct the targeting ligand itself, in addition to synthetic procedures designed to link the molecule to a suitable ligase targeting fragment according to procedures fully described herein above. The compound of formula (XXXIII), wherein B(OR^{x})₂ defines either a boronic acid or ester (including cyclic boronic esters such as pinacol esters), is another embodiment accessible by a Mitsunobu reaction. In this embodiment, the aryl ring is a fragment of the targeting ligand (which will require further elaboration), to which the Mitsunobu reaction appends some linker elements according to the definitions defined herein above.

Aryl dihydro uracil derivatives such as compounds of formula (VIIId/VIIIe), (VIIIg), (VIIIh), (XV) (XVI), (XIX), and (XXV) may be synthesized according to Scheme 16 from the corresponding amines (XXXIV), (XXXIVa), (XXXIVb), (XXXV), (XXXVI), (XXXVII), and (XXXVIII), respectively. The transformation proceeds through a conjugate addition to acrylic acid usually by heating above 70° C. with a co-solvent such as water, followed by reaction with urea and acetic acid, also at elevated temperature such as 120° C., to form the dihydrouracil. In the case of (VIIIg), the dihydrouracil formation may be carried out on the corresponding phenolic acetate ester (XXXIVa) and the ester can be hydrolyzed using acidic conditions, such as HCl treatment in a final step.

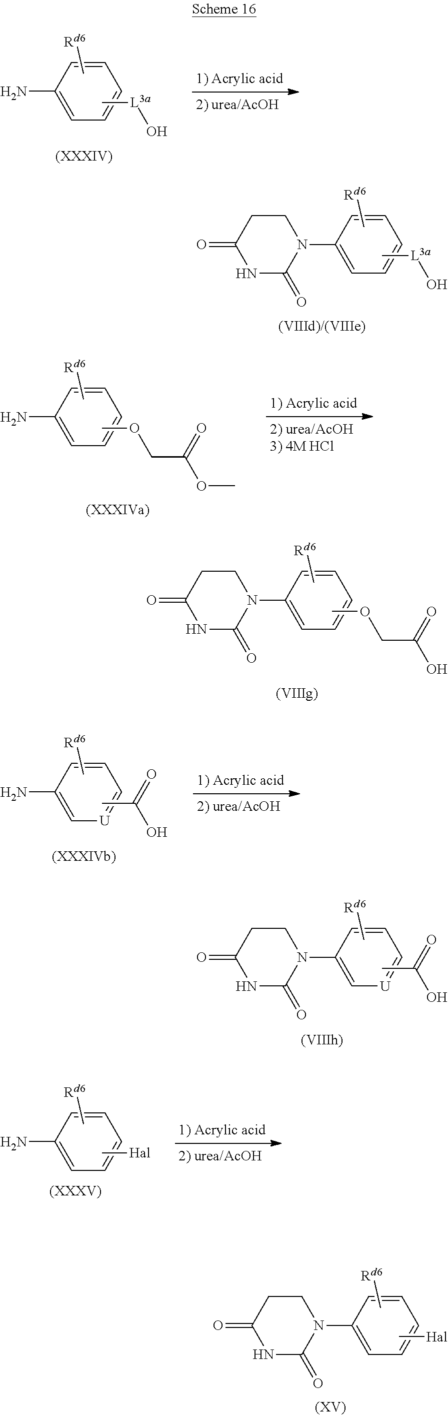

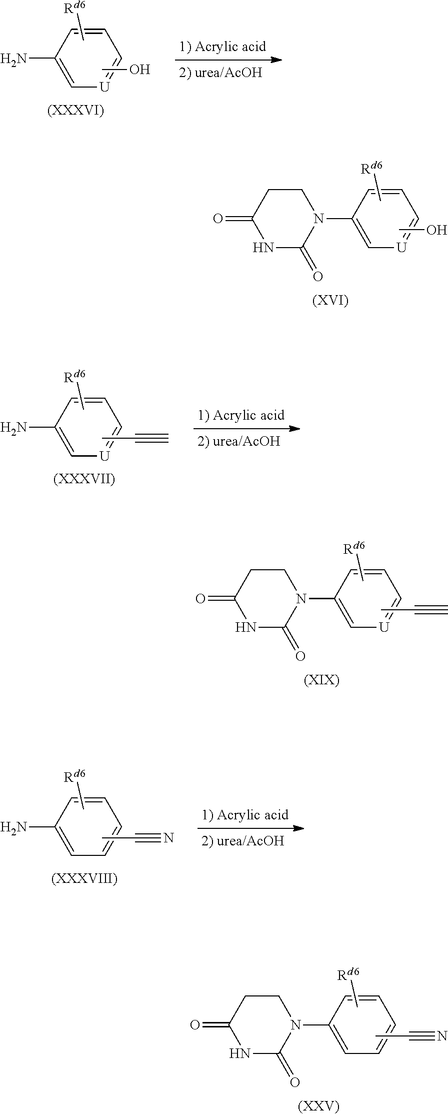

The compounds of formula (XXXIVa) are available from aminophenols with a protected nitrogen (XXXIX), for example a Boc-protected nitrogen, in two steps according to Scheme 17. First, alkylation of the phenol using a base such as Cs₂CO₃ and a 2-haloacetate ester, such as methyl bromoacetate, in a solvent such as acetone with an additive such as potassium iodide provides an intermediate that can undergo N-deprotection using for example an acid such as TFA in a solvent such as dioxane to provide compounds of formula (XXXIXa). Also according to Scheme 17, dihydrouracil intermediates (IXg) can be synthesized, for example, by applying the dihydrouracil forming chemistry to an allyloxy aniline such as (XXXX). Oxidative cleavage of the allyl group using for example an ozonolysis reaction, provides the aldehydes of formula (IXg). Dihydrouracil intermediates (IVj) bearing a sulfonamide linker chain can be synthesized from a compound of formula (XXXXI) in a similar method as for other dihydrouracil building blocks, followed by a deprotection reaction.

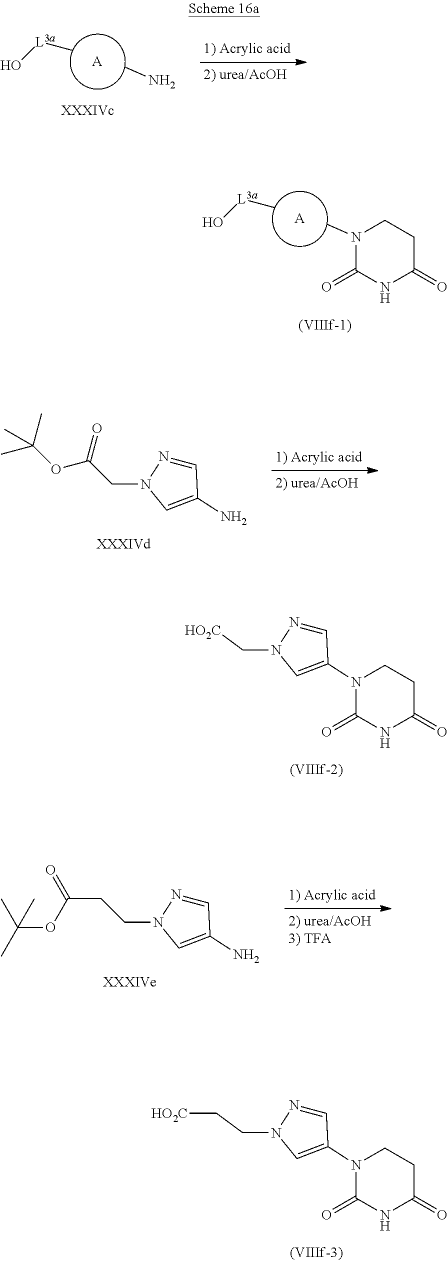

Heteroaryl dihydrouracil derivatives (VIIIf-1) bearing a carboxylic acid functionality, wherein A is a 5- or 6-membered heteroaryl ring may be made according to Scheme 16a using an analogous reaction sequence to that described in Scheme 16. In this case, reaction of the corresponding amino acid (XXXIVc) or a derivative (e.g., such as an amino ester) with acrylic acid at or above 70° C. with a co-solvent, e.g., such as water, followed by reaction with urea and acetic acid, also at an elevated temperature such as 100° C. provides the heteroaryl dihydrouracil (VIIIf-1). Particular examples are the aminopyrazole derivatives (VIIIf-2) and (VIIIf-3), produced from the aminopyrazole tert-butyl ester derivatives (XXXIVd) and (XXXIVe), respectively. In some cases, such as for (VIIIf-2), the reaction conditions result in the concomitant hydrolysis of the tert-butyl ester to the carboxylic acid; for other cases, such as (VIIIf-3) a separate hydrolysis step using an acid such as TFA may be required to produce the free carboxylic acid.

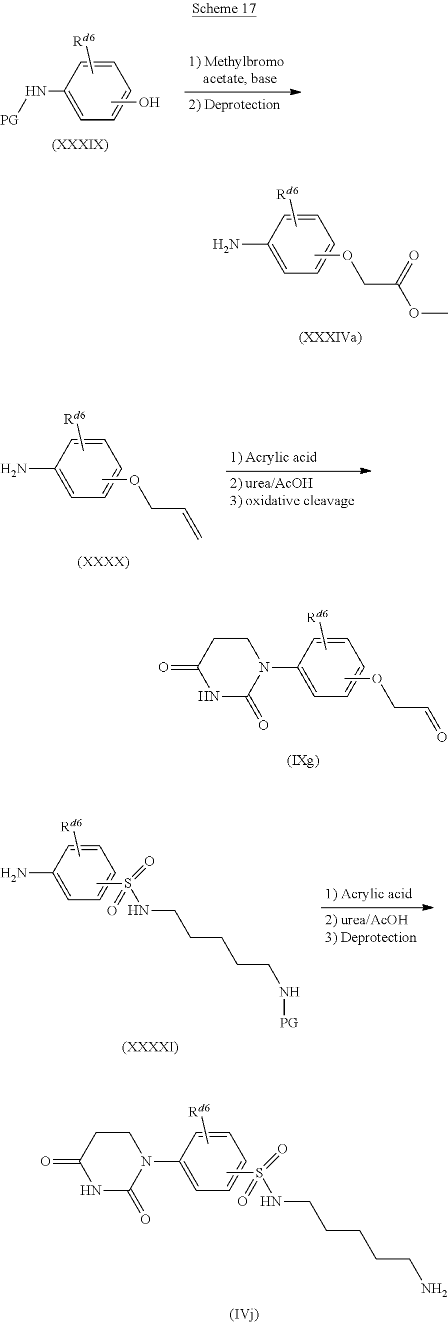

A compound of Formula (XXXXVII), an embodiment of compounds (IXc), may be derived from a compound of Formula (XXXXII) using an oxidative cleavage reaction, such as an ozonolysis, as shown in Scheme 18. Compounds of Formula (XXXXII) may be derived from the corresponding amine of Formula (XXXXIII) through conjugate addition of the amine to acrylic acid, followed by reaction with urea and acetic acid to form the dihydrouracil using conditions already described herein above. Amines of Formula (XXXXIII) may be derived from 3-cyanopyridin-2-one by first reducing the nitrile using conditions such as hydrogenation in the presence of Raney-Nickel in methanol/ammonia solution, then protecting the nitrogen to provide the compound of Formula (XXXXIV), for example, with a typical amine protecting group such as a tert-butoxycarbonyl group. Alkylation of Intermediate (XXXXIV) with an alkylating agent such as allyl bromide and a base such as potassium carbonate in a solvent such as DMF followed deprotection using, for example, HCl in a solvent mixture of DCM and dioxane provides the compound of Formula (XXXXIII). Alternatively, compounds of Formula (XXXXVII) may be synthesized from a compound of Formula (XXXXIV) through alkylation using an alkylating agent containing a protected alcohol to produce followed by removal of the protecting group PG to provide a molecule with Formula (XXXXV). Dihydrouracil formation using the method previously described provides compounds of Formula (XXXXVI). Alcohol deprotection followed by oxidation to the aldehyde using an oxidant such as Dess-Martin periodinane provides the compound of Formula (XXXXVII).

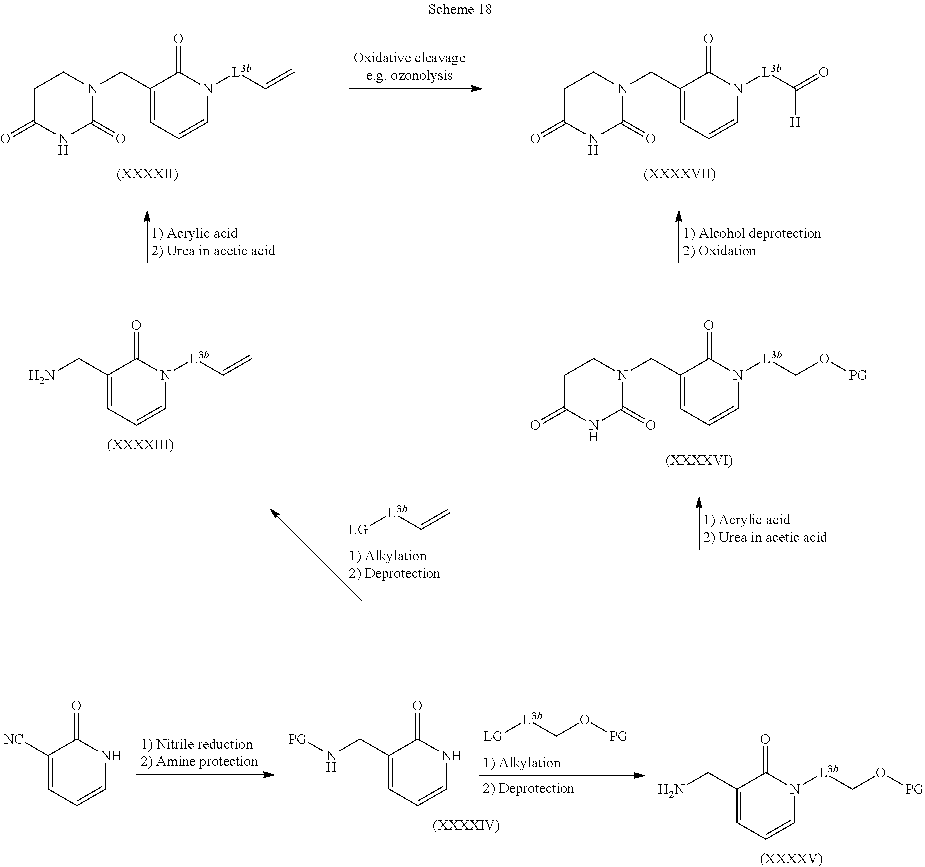

Two alternative methods are described in scheme 19 and scheme 20 to exemplify the synthesis of Intermediates with the formula (ILB III). Hydrogenation of 2,4-dihydroxypyrimidines of formula (XXXXVIII) under pressure (e.g., 30 psi) using a catalyst such as Rhodium on charcoal in a solvent such as water followed by acetylation with PMBCl in a solvent mixture such as DMSO/DCM in the presence of a base such as Cs₂CO₃ provides compounds of formula (XXXXIX) as depicted in scheme 19. Subsequent copper catalyzed arylation of (XXXXIX) using heteroaryl substrates (L) or (LI), wherein Hal represents a halogen atom, preferentially bromine or iodine provides compounds of formula (ILB IIIa) and (ILB IIIb) respectively. Suitable conditions use a ligand, such as DMEDA and a base such as K₂CO₃ in a solvent such as DMF; subsequent deprotection occurs under acidic conditions, such as TFA/TfOH.

For compounds of formula (L) and (LI), U¹, U², U³, U⁴, U⁵, V¹, V², V³, V⁴ and Z¹ are as previously defined herein above.

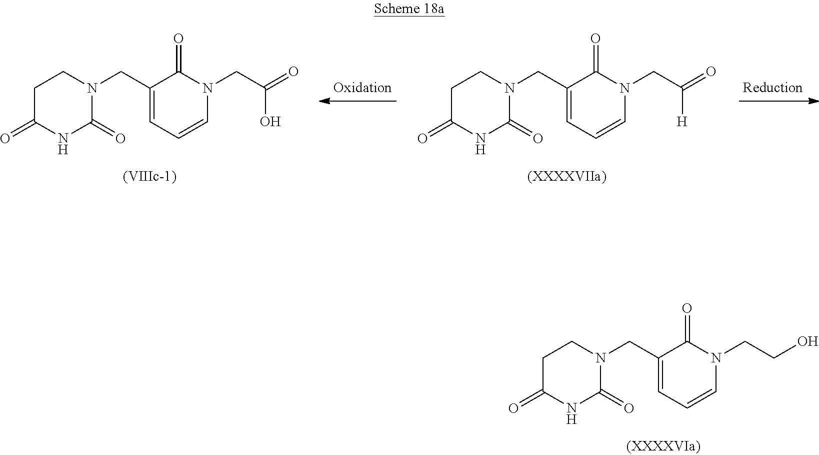 [SMILES: O=C(O)Cn1cccc(CN2CCC(=O)NC2=O)c1=O ; O=CCn1cccc(CN2CCC(=O)NC2=O)c1=O ; O=C1CCN(Cc2cccn(CCO)c2=O)C(=O)N1]

Aldehydes of compound classes (XXXXVII)/(IXc) such as the example (XXXXVIIa) may undergo oxidation, for example by treatment with potassium permanganate in THF at room temperature to give the corresponding carboxylic acid (VIIIc-1), or reduction, for example using sodium borohydride in THF at room temperature to provide the alcohol derivative (XXXXVIa), according to Scheme 18a.

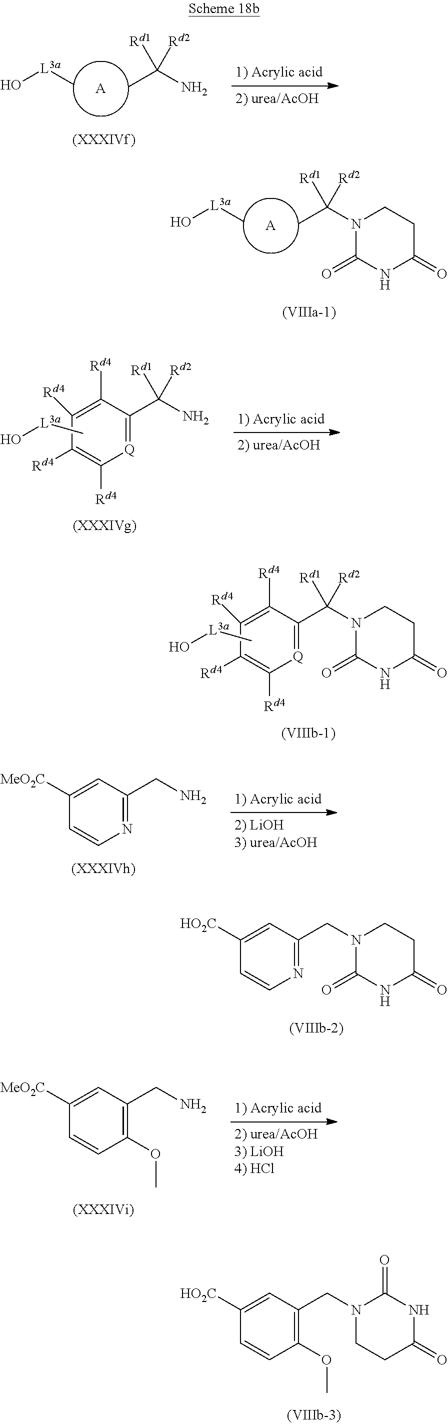

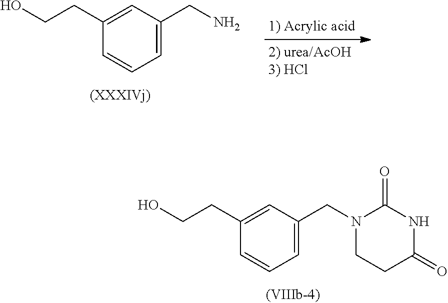 [SMILES: NCc1cccc(CCO)c1 ; O=C1CCN(Cc2cccc(CCO)c2)C(=O)N1]

Benzylic and heterobenzylic dihydrouracil compounds bearing a carboxylic acid functionality belonging to classes (VIIIa)/(VIIIb), may be synthesized according to Scheme 18b. Reaction of the amino acid (XXXIVf) or (XXXIVg) or a derivative (such as an amino ester) with acrylic acid at or above 70° C. with co-solvents such as water and MeCN, or toluene, followed by reaction with urea and acetic acid, also at an elevated temperature such as 100° C. provides the dihydrouracils (VIIIa-1) and (VIIIb-1), respectively.

Representative examples are the derivatives (VIIIb-2), (VIIIb-3) and (VIIIb-4), produced from the amines (XXXIVh), (XXXIVi) and (XXXIVj), respectively. To those skilled in the art, it will be clear that protecting group removal may be effected at different stages of the synthesis, such as for (VIIIb-2) where hydrolysis of the ester using LiOH may be carried out prior to the cyclisation reaction, or such as for (VIIIb-3), where hydrolysis of the ester may be carried out after the cyclisation reaction. In the latter case, ring opening of the dihydrouracil necessitated a further treatment with acid to reform the dihydrouracil ring. Furthermore, use of a primary alcohol in this reaction sequence as for example (VIIIb-4) required a subsequent treatment with aqueous acid to hydrolyse the acetate ester that formed during the dihydrouracil cyclisation reaction.

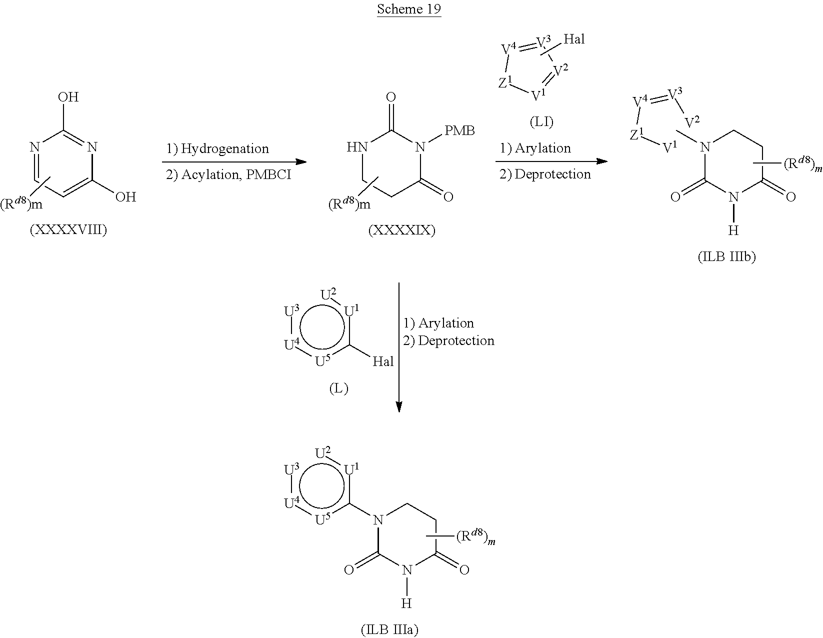

In an embodiment, the same reaction sequence has been applied to prepare structures of formula (ILB IIIc), wherein one of U⁴ or U² is a nitrogen atom according to Scheme 19a. In certain cases, the deprotection of the PMB group also leads to deprotection of other functionality in the aromatic substituent, an example being debenzylation of a benzyl ether.

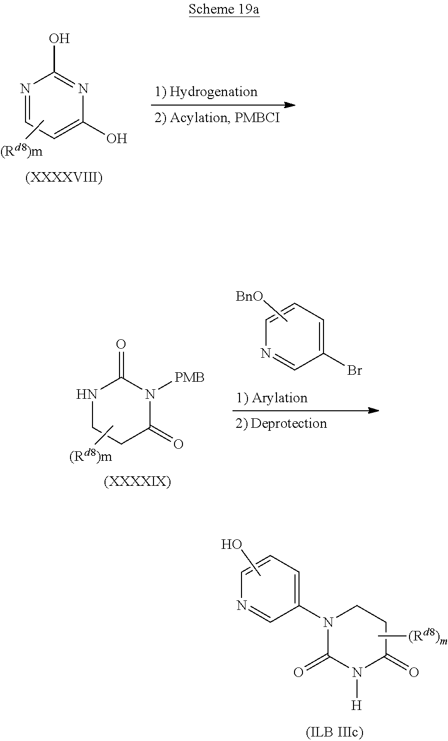

An alternative way to synthesize ILB III is shown in Scheme 20. An amide coupling of an N-protected amino acid (LII) with dimethoxybenzylamine using standard coupling reagents such as CDI in an aprotic solvent such as DCM, followed by Boc-deprotection under acidic conditions with e.g., HCl in ethyl acetate provides compounds of formula (LIII). Cyclization to a compound of formula (LIV) occurs by reacting (LIII) with CDI in the presence of a base such as DIEA in an aprotic solvent such as DCE. A copper-catalyzed arylation of (LIV) using 6-membered heterocycles of formula (L) or 5-membered heterocycles of formula (LI) in the presence of a ligand and a catalyst under conditions already described herein above (in Scheme 19) provide compounds of formula ILB IIId and ILB IIIe respectively.

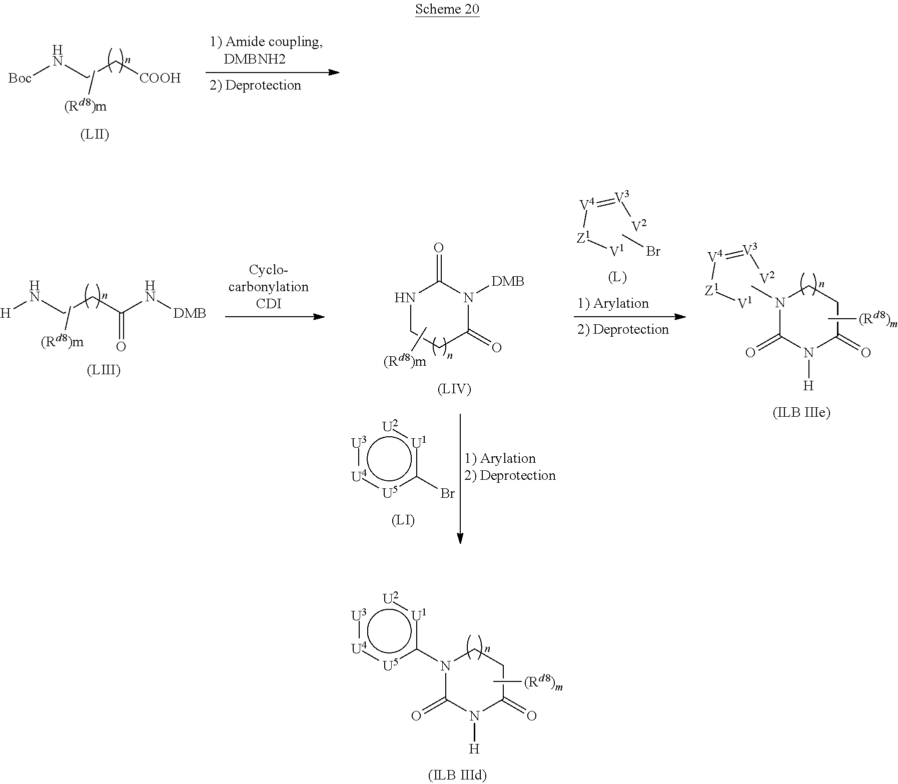

Targeting ligands can be synthesized using a wide variety of methods. In an embodiment, a compound of formula (IIIa) is synthesized by a palladium-catalyzed coupling reaction, such as a Suzuki reaction between a compound of formula (LV) and a compound of formula (LVI), using a catalyst (e.g., PdCl₂(dppf)) and a base (e.g., Cs₂CO₃) in a solvent mixture (e.g., dioxane/water), according to Scheme 21. Compound (LV) may be made from the ester (LVII), by reduction to the alcohol using a reductant such as LiAlH₄ in a solvent such as THF, followed by oxidation to the aldehyde using MnO₂ in THF.

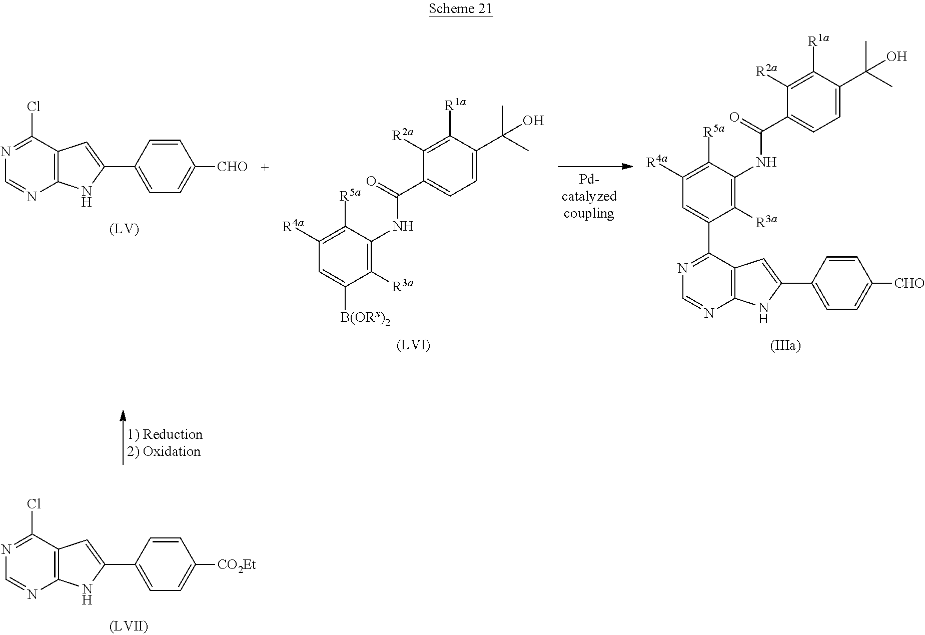

Synthesis of other specific intermediates containing targeting ligands are described in the experimental section. The TNNI3K targeting binding moiety has been prepared by analogy to a literature procedure. See WO 2011/56740. The reported advanced intermediate 2-((6-chloro-2-methylpyrimidin-4-yl)amino)-N-(2-chloro-6-methylphenyl)thiazole-5-carboxamide (CAS No. [302964-08-5]) was also utilized in the preparation of bifunctional compounds.

The compounds of formula (III), (IIIa), (V), (VI), (XXIV), (XXII), (XXIIa), and (XXVIII) which contain targeting ligands and appropriate functional groups for attaching to a linker and ligase targeting ligand, can be prepared by a range of standard synthetic methods and procedures either known to those skilled in the art, or which will be apparent to the skilled chemist in light of the teachings herein. It is understood that depending on the nature of the targeting ligand it is possible to apply similar targeting ligands but with differing functional groups to the synthesis of the compounds of this invention. Thus, compounds such as (III), (V), (VI), (XXIV), (XXII) and (XXVIII) may be interconverted using functional group interconversions well known to those skilled in organic synthesis.

A mixture of enantiomers, diastereomers, and cis/trans isomers resulting from the process described above can be separated into their single components by chiral salt technique, chromatography using normal phase, reverse phase or chiral column, depending on the nature of the separation.

Any resulting racemates of compounds of the present disclosure or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid, or camphor-10-sulfonic acid. Racemic compounds of the present disclosure or racemic intermediates can also be resolved by chiral chromatography, e.g., high pressure liquid chromatography (HPLC) using a chiral adsorbent.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

It should be understood that in the description and formula shown above, the various groups and variables are as previously defined herein above, except where otherwise indicated. Furthermore, for synthetic purposes, the compounds of schemes 1, la, 1b, 2-4, 4a, 5, 5a, 6, 6a, 7-16, 16a, 17-18, 18a, 18b, 19, 19a, and 20-21 are merely representative with elected radicals to illustrate the general synthetic methodology of the compounds disclosed herein. The preparation of specific intermediates and examples using the general methods described above is provided in detail in the experimental section.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof, which may suggest themselves to those, skilled in the art without departing from the spirit of the present disclosure and/or scope of the appended claims.

Compounds described herein may be prepared by methods known in the art of organic synthesis. In all of the methods it is understood that protecting groups for sensitive or reactive groups may be employed where necessary in accordance with general principles of chemistry.

Protecting groups are manipulated according to standard methods of organic synthesis. See, e.g., T. W. Green and P. G. M. Wuts, Protective Groups in Organic Synthesis, 3^{rd} edition, John Wiley & Sons (1999). These groups are removed at a convenient stage of the compound synthesis using methods that are readily apparent to those skilled in the art.

Temperatures are given in degree Celsius. Abbreviations used are those conventional in the art and listed below.

All starting materials, building blocks, reagents, acids, bases, dehydrating agents, solvents, and catalysts utilized to synthesize the compounds of the present invention are either commercially available or can be produced by organic synthesis methods known to one of ordinary skill in the art. Further, the compounds of the present invention can be produced by organic synthesis methods known to one of ordinary skill in the art as shown in the following examples.

### Abbreviations

ACN acetonitrile
AcOH acetic acid
app. apparent
aq. aqueous
ATP adenosine 5′-triphosphate
BINAP racemic 2,2′-bis(diphenylphosphino)-1,1′-binaphthyl
BISPIN bis(pinacolato)diboron
BOC tert-butoxycarbonyl
br broad
BSA bovine serum albumin
CDI carbonyldiimidazole
CHX cyclohexane
conc concentrated
d doublet
dd doublet of doublets
DCE 1,2-dichloroethane
DCM Dichloromethane
DEA diethylamine
DEAD diethyl azodicarboxylate
DIAD diisopropyl azodicarboxylate
DIBAL-H diisobutyl aluminum hydride
DIEA diethylisopropylamine
DIPEA diisopropylethylamine
DMA Dimethyl acetamide
DMBNH₂ 2,4-dimethoxy-benzyl chloride
DME 1,4-dimethoxyethane
DMEDA 1,2-dimethylethylenediamine
DMF N,N-dimethylformamide
DMPU 1,3-dimethyl-3,4,5,6-tetrahydro-2(1H)-pyrimidinone
DMSO dimethylsulfoxide
Dppf 1,1′-bis(diphenylphosphino)ferrocene
EDTA ethylenediamine tetraacetic acid
e.g. for example
eq. equivalent
ESI electrospray ionization
Et₂O diethylether
EtOAc ethyl acetate
EtOH ethanol
h hour(s)
GC gas chromatography
HATU 1-[bis(dimethylamino)methylene]-1,2,3-triazolo[4,5-b]pyridinium 3-oxide hexafluorophosphate
HBTU 2-(1H-benzotriazol-1-yl)-1,1,3,3-tetramethyluronium hexafluorophosphate
HCl hydrogen chloride
HCOOH formic acid
H₂O water
HOAc acetic acid
HOBt 1-hydroxy-7-azabenzotriazole
HPLC high performance liquid chromatography
HV high vacuum
iPrOH isopropanol
K kelvin
KOAc potassium acetate
L liter
LC-MS liquid chromatography and mass spectrometry
LiHMDS lithium bis(trimethylsilyl)amide
m multiplet
M molar
MeOH methanol
mg milligram
MgSO₄ magnesium sulfate
MHz megahertz
min minute(s)
mL milliliter
mm millimeter
μm micrometer
mmol millimol
mM millimolar
MS mass spectrometry
Ms methanesulfonyl
MsCl methanesulfonyl chloride
Ms₂O methanesulfonic anhydride
MTBE methyl tert-butyl ether
MW molecular weight
m/z mass to charge ratio
NaBH₄ sodium borohydride
NaBH₃CN sodium cyanoborohydride
NaBH(OAc)₃ sodium triacetoxyborohydride
NaH sodium hydride
NaHCO₃ sodium bicarbonate
NaOAc sodium acetate
NaOH sodium hydroxide
NH₄Cl ammonium chloride
NH₄OAc ammonium acetate
NH₄OH ammonium hydroxide
NMM N-methylmorpholine
NMP N-methyl-2-pyrrolidine
NMR nuclear magnetic resonance
OAc acetate
PdCl₂(dppf) 1,1′-Bis(diphenylphosphino)ferrocene-palladium(II)dichloride
PdCl₂(dppf)-CH₂Cl₂ 1,1′-Bis(diphenylphosphino)ferrocene-palladium(II)dichloride dichloromethane complex
PdCl₂(PPh₃)₂ bis(triphenylphosphine)palladium dichloride
Pd(PPh₃)₄ tetrakis(triphenylphosphine)palladium
PE petroleum ether
PG protecting group
PMBCl para-methoxy-benzyl chloride
PPh₃ triphenylphosphine
ppm parts per million
PyBOP benzotriazol-1-yloxytripyrrolidinophosphonium
hexafluorophosphate
rac racemic
RM reaction mixture
Rt retention time
RT room temperature
s singlet
sat. saturated
SEM-Cl 2-(trimethylsilyl)ethoxymethyl chloride
SFC supercritical fluid chromatography
t triplet
t-BuOH tert-butyl alcohol
t-BuOK potassium tert-butoxide
TBTU O-(benzotriazol-1-yl)-N,N,N′,N′-tetramethyluronium tetrafluoroborate
TBDPS tert-butyl diphenylsilyl
TEA triethylamine
TFA trifluoroacetic acid
THF tetrahydrofuran
Tris-HCl aminotris(hydroxymethyl)methane hydrochloride

### Example 1

### Analytical Methods

### General Conditions

### NMR

NMR spectra were run on Bruker AVANCE 400 MHz or 500 MHz NMR spectrometers using ICON-NMR, under TopSpin program control. Spectra were measured at 298 K, unless indicated otherwise, and were referenced relative to the solvent resonance.

### LC-MS

Mass spectra were acquired on LC-MS, SFC-MS, or GC-MS systems using electrospray, chemical and electron impact ionization methods from a range of instruments of the following configurations: Waters Acquity UPLC/SQD system, using a photodiode array detector and a single quadrupole mass detector or Agilent 1200 systems with G 6110 series mass detector. [M+H]⁺ refers to protonated molecular ion of the chemical species.

### Method LCMS1

Column: ACQUITY UPLC® HSS T3 (2.1×50 mm, 1.8 μm)
Column temperature: 60° C.
Eluents: A: water+0.05% FA+3.75 mM AA
B: ACN+0.04% FA
Flow rate: 1.0 mL/min
Gradient: 5% to 98% B in 1.4 min

### Method LCMS2

Column: ACQUITY UPLC® HSS T3 (2.1×50 mm, 1.8 μm)
Column temperature: 60° C.
Eluents: A: water+0.05% FA+3.75 mM AA
B: ACN+0.04% FA
Flow rate: 1.0 mL/min
Gradient: concave from 1% to 98% B in 1.4 min

### Method LCMS3

Column: ACQUITY UPLC® BEH C18 (2.1×50 mm, 1.7 μm)
Column temperature: 80° C.
Eluents: A: water+0.05% FA+3.75 mM AA
B: iPrOH+0.05% FA
Flow rate: 0.6 mL/min
Gradient: from 5% to 98% B in 1.7 min

### Method LCMS4

Column: XBridge® BEH™ C18 (2.1×50 mm, 2.5 μm)
Column temperature: 80° C.
Eluents: A: water+5 mM NH4OH
B: ACN+5 mM NH4OH
Flow rate: 1.0 mL/min
Gradient: from 2% to 98% B in 1.4 min

### Method LCMS5

Column: ACQUITY UPLC® HSS T3 (2.1×100 mm, 1.8 μm)
Column temperature: 60° C.
Eluents: A: water+0.05% FA+3.75 mM AA
B: ACN+0.04% FA
Flow rate: 0.8 mL/min
Gradient: 5% to 98% B in 9.4 min

### Method LCMS6

Column: ACQUITY UPLC® BEH C18 (2.1×100 mm, 1.7 μm)
Column temperature: 80° C.
Eluents: A: water+0.05% FA+3.75 mM AA
B: iPrOH+0.05% FA
Flow rate: 0.4 mL/min
Gradient: from 5 to 60% B in 8.4 min from 60 to 98% B in 1 min

### Method LCMS7

Column: ACQUITY UPLC® BEH C18 (2.1×50 mm, 1.7 μm)
Column temperature: 80° C.
Eluents: A: water+4.76% iPrOH+0.05% FA+3.75 mM AA
B: iPrOH+0.04% FA
Flow rate: 0.6 mL/min
Gradient: from 1% to 98% B in 1.7 min

### Method LCMS8

Column: Kinetex Evo C18 (2.1×50 mm, 1.7 μm)
Column temperature: 60° C.
Eluents: A: water+0.05% FA+3.75 mM AA
B: ACN+0.04% FA
Flow rate: 1.0 mL/min
Gradient: 5% to 98% B in 1.4 min

### Method LCMS9

Column: Ascentis® Express C18 2.7 μm 2.1×50 mm
Column temperature: 80° C.
Eluents: A: water+4.76% isopropanol+0.05% FA+3.75 mM AA
B: isopropanol+0.05% FA
Flow rate: 1.0 mL/min
Gradient: 1% to 50% B in 1.4 min, 50 to 98% B in 0.3 min

### Method LCMS10

Column: Kinetex EVO C18 (30*2.1 mm, 5 um)
Column temperature: 50° C.
Eluents: A: 0.0375% TFA in water (v/v)
   B: 0.01875% TFA in Acetonitrile (v/v)
Flow rate: 1.5 ml/min
Gradient: 0% to 60% B in 1.55 min

### Method LCMS11

Column: Kinetex EVO C18 (30*2.1 mm, 5 um)
Column temperature: 50° C.
Eluents: A: 0.0375% TFA in water (v/v)
   B: 0.01875% TFA in Acetonitrile (v/v)
Flow rate: 1.5 ml/min
Gradient: 0% to 60% B in 7 min

### Method LCMS12

Column: CORTECS™ C18+ 2.7 μm
Column temperature: 80.0° C.
Eluents: A: water+4.76% isopropanol+0.05% FA+3.75 mM AA
   B: isopropanol+0.05% FA
Flow rate: 1.0 mL/min
Gradient: from 1 to 50% B in 1.4 min; 50 to 98% B in 0.3 min

### Method LCMS13

Column: Waters XSelect HSS T3 3.5 um 4.6*50 mm
Column temperature: 50° C.
Eluents: A: 0.0375% TFA in water (v/v)
   B: 0.01875% TFA in ACN (v/v)
Flow rate: 1 ml/min
Gradient: 0% to 30% B in 5 min

### Example 2

### Analytical Methods

### General Conditions:

### NMR

NMR spectra were run on Bruker AVANCE 400 MHz or 500 MHz NMR spectrometers using ICON-NMR, under TopSpin program control. Spectra were measured at 298K, unless indicated otherwise, and were referenced relative to the solvent resonance.

### LC-MS

Mass spectra were acquired on LC-MS, SFC-MS, or GC-MS systems using electrospray, chemical and electron impact ionization methods from a range of instruments of the following configurations: Waters Acquity UPLC/SQD system, using a photodiode array detector and a single quadrupole mass detector or Agilent 1200 systems with G 6110 series mass detector. [M+H]⁺ refers to protonated molecular ion of the chemical species.

### Method A

Column: XBridge C18 (4.6×50 mm, 3.5 μm)
Column temperature: 50° C.
Eluents: A: aq. ammonium hydrogen carbonate (10 mM); B: ACN
Flow rate: 1.8 mL/min
Gradient: 5% to 95% B in 1.5 min

### Method B

Column: SunFire C18 (4.6×50 mm, 3.5 μm)
Column temperature: 50° C.
Eluents: A: aq. TFA (0.01%); B: ACN containing TFA (0.01%)
Flow rate: 2.0 mL/min
Gradient: 5% to 95% B in 1.4 min

### Method C

Column: SunFire C18 (4.6×50 mm, 3.5 μm)
Column temperature: 50° C.
Eluents: A: aq. TFA (0.01%); B: ACN containing TFA (0.01%)
Flow rate: 2.0 mL/min
Gradient: 5% to 95% B in 1.3 min

### Method D

Column: HALO C18 (4.6×30 mm, 2.7 μm)
Column temperature: 50° C.
Eluents: A: aq. TFA (0.01%); B: ACN containing TFA (0.01%)
Flow rate: 2.2 mL/min
Gradient: 5% to 95% B in 1.0 min

### Method E

Column: SunFire C18 (4.6×50 mm, 3.5 μm)
Column temperature: 50° C.
Eluents: A: aq. TFA (0.01%); B: ACN containing TFA (0.01%)
Flow rate: 2.0 mL/min
Gradient: 5% to 95% B in 1.2 min

### Method F

Column: SunFire C18 (4.6×50 mm, 3.5 μm)
Column temperature: 50° C.
Eluents: A: aq. TFA (0.01%); B: ACN containing TFA (0.01%)
Flow rate: 2.0 mL/min
Gradient: 5% to 95% B in 1.2 min, followed by 95% B for 1.3 min

### Method G

Column: XBridge C18 (4.6×50 mm, 3.5 μm)
Column temperature: 40° C.
Eluents: A: aq. ammonium hydrogen carbonate (10 mM); B: CAN
Flow rate: 1.8 mL/min
Gradient: 5% to 95% B in 1.4 min, followed by 95% B for 1.6 min

### Method H

Column: XBridge C18 (4.6×50 mm, 3.5 μm)
Column temperature: 40° C.
Eluents: A: aq. ammonium hydrogen carbonate (10 mM); B: ACN
Flow rate: 2.0 mL/min
Gradient: 5% to 95% B in 1.5 min

### Preparative Chromatography Methods

Normal and reverse phase chromatography purifications have been performed on a Biotage Isolera One system.

### Achiral Preparative HPLC Methods

Method PB with Basic Modifier
Instrument: Gilson 281 (PHG012)
Column: Xtimate C18 (21.2×250 mm, 10 μm)
Column temperature: RT
Eluents: A: aq. ammonium hydrogen carbonate (10 mM); B: ACN
Flow: 30 mL/min
Detection: UV @ 254 nm, 214 nm
   Method PA with Acidic Modifier
Instrument: Gilson 281 (PHG012)
Column: Xtimate C18 (21.2×250 mm, 10 μm)
Column temperature: RT
Eluents: A: aq. TFA (0.1%); B: ACN
Flow: 30 mL/min
Detection: UV @ 254 nm, 214 nm
   Method PA2 with Acidic Modifier
Column: Waters Xbridge (150*25*10 μm)
Column temperature: 25° C.
Eluents: A: aq. TFA (0.1%); B: ACN
Flow rate: 25.0 ml/min
Gradient: 34% to 54% B in 10 min
   Method PA3 with Acidic Modifier
Instrument: Gilson GX-215&Shimadzu LCMS2020
Column: Waters Atlantis T3 150*30 mm*5 um
Column temperature: RT
Eluents: A: aq. TFA (0.1%); B: ACN
Flow: 25 mL/min
Detection: UV @ 254 nm, 220 nm

### Example 3

### Analytical Methods

### General Conditions

### NMR

NMR spectra were recorded on Bruker AVANCE 400 MHz, 500 MHz or 600 MHz NMR spectrometers using ICON-NMR, under TopSpin program control. Spectra were measured at 298 K, unless indicated otherwise, and were referenced relative to the solvent resonance according to the values described in J. Org. Chem. 62: 7512-7515 (1997) (e.g. DMSO d6 at 2.50 ppm, CDCl₃ at 7.26 ppm, D₂O at 4.79 ppm and MeOD-d4 at 3.31 ppm). Significant peaks are tabulated in the following order: multiplicity (s, singlet; d, doublet; t, triplet; q, quartet; m, multiplet; br, broad; v, very) and number of protons.

### LC-MS

Mass spectra were acquired on LC-MS, SFC-MS, or GC-MS systems using electrospray, chemical and electron impact ionization methods from a range of instruments of the following configurations: Waters Acquity UPLC/SQD system, using a photodiode array detector and a single quadrupole mass detector or Agilent 1200 systems with G 6110 series Mass Spectrometer. [M+H]⁺ refers to the protonated molecular ion of the chemical species.

### Method XA

Column: Waters Acquity HSS T3 1.8 μm 2.1×50 mm or 2.1×100 mm
Column temperature: 60° C.
Eluents: A: aq. formic acid (0.05%)+aq. ammonium acetate (3.75 mM); B: ACN containing formic acid (0.04%)
Flow rate: 1.0 mL/min
Gradient: 5% to 98% B in 1.4 min

### Method XB

Column: Waters Acquity HSS T3 1.8 μm 2.1×50 mm or 2.1×100 mm
Column temperature: 60° C.
Eluents: A: aq. formic acid (0.05%)+aq. ammonium acetate (3.75 mM); B: ACN containing formic acid (0.04%)
Flow rate: 0.8 mL/min
Gradient: 5% to 98% B in 9.4 min

### Method XC

Column: Waters Acquity HSS T3 1.8 μm 2.1×50 mm or 2.1×100 mm
Column temperature: 50° C.
Eluents: A: aq. formic acid (0.05%)+aq. ammonium acetate (3.75 mM); B: ACN containing
formic acid (0.04%)
Flow rate: 1.2 mL/min
Gradient: 2% to 98% B in 1.4 min

### Method XD

Column: SunFire C18, 4.6×50 mm, 3.5 μm
Column temperature: 50° C.
Eluents: A: aq. TFA (0.01%); B: acetonitrile containing TFA (0.01%)
Flow rate: 2.0 mL/min
Gradient: 5% to 95% B in 1.4 min

### Method XE

Column: SunFire C18, 4.6×50 mm, 3.5 μm
Column temperature: 50° C.
Eluents: A: aq. TFA (0.01%); B: acetonitrile containing TFA (0.01%)
Flow rate: 2.0 mL/min
Gradient: 5% to 95% B in 1.2 min, 95% B for 1.3 min

### Method XF

Column: Phenomenex, 3.0×30 mm, 5 μm
Column temperature; 50° C.
Eluents: A: aq. ammonium hydrogen carbonate (10 mM); B: acetonitrile
Flow rate: 1.5 mL/min
Gradient: 5% to 95% B in 1.5 min, 95% B for 0.7 min

### Method XG

Column: XBridge C18, 4.6×50 mm, 3.5 μm
Column temperature: 40° C.
Eluents: A: aq. ammonium hydrogen carbonate (10 mM); B: acetonitrile
Flow rate: 2.0 mL/min
Gradient: 5% to 95% B in 1.5 min

### Method XH

Column: XBridge C18, 4.6×50 mm, 3.5 μm
Column temperature: 50° C.
Eluents: A: aq. ammonium hydrogen carbonate (10 mM); B: acetonitrile
Flow rate: 1.8 mL/min
Gradient: 5% to 95% B in 1.5 min, 95% B for 1.5 min

### Method XI

Column: SunFire C18, 3×30 mm, 2.5 μm
Column temperature: 50° C.
Eluents: A: aq. TFA (0.01%); B: acetonitrile containing TFA (0.01%)
Flow rate: 1.5 mL/min
Gradient: 5% to 95% B in 1.5 min

### Method XJ

Column: XBridge C18, 4.6×50 mm, 3.5 μm
Column temperature: 40° C.
Eluents: A: aq. ammonium hydrogen carbonate (10 mM); B: acetonitrile
Flow rate: 1.8 mL/min
Gradient: 5% to 95% B in 1.4 min, 95% B for 1.6 min

### Chiral Analytical HPLC Methods

### Method XK

Instrument: Agilent 1200 system
Column: Chiralpak ID 5 um 4.6×250 mm
Column temperature: RT
Eluents: Hept:DCM:MeOH (40:35:25)+DEA (0.1%)
Flow rate: 1.0 mL/min
Gradient: isocratic
Detection: UV at 254 nm

### Preparative Chromatography Methods

Normal and reverse phase chromatography purifications have been performed on a CombiFlash Rf200 or Rf+ system. Alternatively, chromatography purifications on reverse phase have been performed on an Interchim Puriflash 4250 system.

Achiral SFC Chromatography separations have been performed using a Waters Preparative SFC-100-MS system with either a Waters 2998 Photodiode Array Detector or a Waters MS Single Quadrupole Detection using MeOH as modifier. The back pressure was 120 bar, the flow 100 g CO₂/min and the column temperature 40° C. The type of the column varies and has been indicated in the individual experimental sections. Reverse phase HPLC purifications have been performed on a Waters HPLC Preparative System with either a Waters 2998 Photodiode Array Detector or a Waters MS Single Quadrupole Detection.

### Achiral Preparative HPLC Methods

### Method XL

Instrument: Gilson GX-281
Column: SunFire C18
Column temperature: RT
Mobile phase: ACN in water containing TFA (0.1%)
Flow: 40 mL/min
Detection: UV @ 254 nm

### Chiral Preparative Chromatography Methods

### Method XM

Instrument: Gilson Trilution I HPLC System
Column: ChiralPak ID, 5 μM, 250×20 mm
Column temperature: RT
Mobile phase: heptane/DCM/MeOH (40:35:25) containing DEA (0.05%)
Flow: 10 mL/min
Detection: UV @ 254 nm

### Method XN:

Instrument: Gilson
Column: Reprosil 100 C18 (250×30 mm, 5 μm)
Column temperature: Room temperature
Eluents: A: Water (0.1% TFA), B: ACN
Flow rate: 25 mL/min
Gradient: 0-2 min. 100% Eluent A, 2-26 min. 100% to 5% Eluent A
Detection: UV @ 254 nm

### Method XN-A:

Instrument: Gilson
Column: Reprosil 100 C18 (250×30 mm, 5 μm)
Column temperature: Room temperature
Eluents: A: Water (0.1% TFA), B: ACN
Flow rate: 25 mL/min
Gradient: 0-2 min. 100% Eluent A, 2-26 min. 100% to 50% Eluent A
Detection: UV @ 254 nm

### Method XO

Column: AcQuity UPLC BEH C18, 2.1×30 mm, 1.7 μm
Column temperature: 50° C.
Eluents: A: 5 mM NH₄OH in water; B: 5 mM NH₄OH in ACN
Flow rate: 1.0 mL/min
Gradient: 1% to 30% B in 1.20 min, 30% to 98% B in 0.95 min, 98% to 1% B in 0.04 min

### Method XP

Column: AcQuity UPLC BEH C18, 2.1×50 mm, 1.7 μm
Column temperature: 50° C.
Eluents: A: 5 mM NH₄OH in water; B: 5 mM NH₄OH in ACN
Flow rate: 1.0 mL/min
Gradient: 1% to 30% B in 3.20 min, 30% to 98% B in 1.95 min, 98% to 1% B in 0.04 min

### Method XP-A

Column: AcQuity UPLC BEH C18, 2.1×50 mm, 1.7 μm
Column temperature: 50° C.
Eluents: A: 0.1% Formic Acid in Water
   B: 0.1% Formic Acid in Acetonitrile
Flow rate: 1.0 mL/min
Gradient: 1% to 30% B in 1.20 min, 30% to 98% B in 0.95 min, 98% to 1% B in 0.04 min

### Method XQ

Column: AcQuity UPLC BEH C18 1.7 μm 2.1×30 mm
Column temperature: 50° C.
Eluents: A: 0.1% Formic Acid in Water; B: 0.1% Formic Acid in Acetonitrile
Flow rate: 1.0 mL/min
Gradient: 2% B for 0.10 min, 2% to 98% B in 1.40 min, 98% B for 0.30 min, 98% to 2% B in 0.10 min, 2% B for 0.10 min

### Method XR

Column: AcQuity UPLC BEH C18 1.7 μm 2.1×50 mm
Column temperature: 50° C.
Eluents: A: 5 mM NH₄OH in water; B: 5 mM NH₄OH in ACN
Flow rate: 1.0 mL/min
Gradient: 2% to 98% B in 4.40 min, 98% B for 0.75 min, 98% to 2% B in 0.04 min

### Method XR-A

Column: AcQuity UPLC BEH C18 1.7 μm 2.1×50 mm
Column temperature: 50° C.
Eluents: A: 0.1% Formic Acid in Water
   B: 0.1% Formic Acid in Acetonitrile
Flow rate: 1.0 mL/min
Gradient: 2% to 98% B in 5 min, 98% B for 0.75 min, 98% to 2% B in 0.04 min

### Method XV-B

Column: AcQuity UPLC BEH C18 1.7 μm 2.1×30 mm
Column temperature: 50° C.
Eluents: A: 5 mM Ammonium Hydroxide in Water; B: 5 mM Ammonium Hydroxide in Acetonitrile
Flow rate: 1.0 mL/min
Gradient: 2% to 98% B in 1.80 min

### Method XS

Detection: —Waters 2998 Photodiode Array Detector
   Waters MS Single Quadrupole Detection
Column temperature: RT
Eluent A: water/Eluent B: acetonitrile, both containing 0.1% trifluoroacetic acid

### Method XT

Detection: —Waters 2998 Photodiode Array Detector
   Waters MS Single Quadrupole Detection
Column temperature: RT
Eluent A: water/Eluent B: acetonitrile, both containing 0.1% NH₄OH

### Method XU

Instrument: Waters Preparative SFC-100-MS system
Detection: —Waters 2998 Photodiode Array Detector
   Waters MS Single Quadrupole Detection
Modifier: Methanol
ABPR: 1 20 bar
Column temperature: 40° C.
Flow rate: 100 g/min.

### Method XX

Instrument: Gilson GX-281, Gilson 155, Gilson 331
Column: Sunfire C18 (30×100 mm, 5 μm)
Column temperature: RT
Eluents: A: aq. TFA (0.1%); B: ACN
Flow: 50 mL/min
Detection: UV @ 254 nm, 214 nm

### Materials for Solid Phase Extraction

The following solid phase extraction (SPE) cartridges were used according to product instructions to generate the corresponding free base from different salts:

PL-HCO₃ MP SPE cartridges were purchased from Agilent StratosPhere—Ref: PL-HCO₃ MP-resin, 1.8 mmol/g, 100 A, 150-300 μm, 500 mg, 6 mL.

SCX cartridges were purchased from Agilent-Ref.: HF Mega DE-SCX, 2 g, 12 mL.

### Example 4

### Synthetic Intermediate and Reagent Synthesis

### Intermediate AA: N-(5-(2,4-dioxotetrahydropyrimidin-1(2H)-yl)-2-methylbenzyl)-5-(methylamino)pentanamide

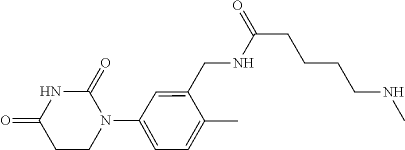 [SMILES: CNCCCCC(=O)NCc1cc(N2CCC(=O)NC2=O)ccc1C]

### Step 1: 3-((5-cyano-2-methylphenyl)amino)propanoic acid

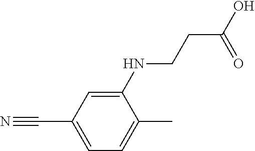

A solution of 3-amino-4-methylbenzonitrile (CAS No. [60710-80-7], 10 g, 75.66 mmol), and Acrylic acid (CAS No. [79-10-7], 3 g, 151.33 mmol), in toluene (25 mL) was stirred at 120° C. for 16 h under N₂. The RM was concentrate to dryness to afford the title compound as a light yellow solid (15 g). Method G: Rt=1.34 min; [M+H]⁺=204.

### Step 2: 3-(2,4-dioxotetrahydropyrimidin-1(2H)-yl)-4-methylbenzonitrile

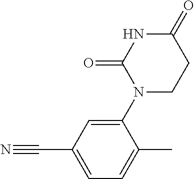 [SMILES: Cc1ccc(C#N)cc1N1CCC(=O)NC1=O]

To a solution of 3-((5-cyano-2-methylphenyl)amino)propanoic acid (7.8 g, 38.19 mmol) and Urea (CAS No. [57-13-6], 11.5 g, 191 mmol) in AcOH (CAS No. [64-19-7], 100 mL) was stirred at 120° C. for 16 h. The RM was poured on crushed ice (200 g), stirred for 30 min and filtered to afford the title compound as a light yellow solid (4 g). Method F: Rt=1.31 min; [M+H]⁺=230.

### Step 3: 1-(5-(aminomethyl)-2-methylphenyl)dihydropyrimidine-2,4(1H,3H)-dione

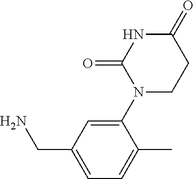 [SMILES: Cc1ccc(CN)cc1N1CCC(=O)NC1=O]

A solution of 3-(2,4-dioxotetrahydropyrimidin-1(2H)-yl)-4-methylbenzonitrile (4 g, 17.45 mmol) and Raney Nickel (500 mg) in MeOH/NH₄OH (1000 mL/200 mL) was stirred under H₂ at RT for 16 h. The mixture was filtered through Celite® filter aid and concentrated to dryness. The crude compound was purified by reverse phase HPLC Method (5% to 95% ACN/H₂O, 0.01% TFA) to give the title compound as the TFA salt (1.1 g). Method F: Rt=0.379 min; [M+H]⁺=234.

### Step 4: tert-butyl (5-((5-(2,4-dioxotetrahydropyrimidin-1(2H)-yl)-2-methylbenzyl)amino)-5-oxopentyl)(methyl)carbamate

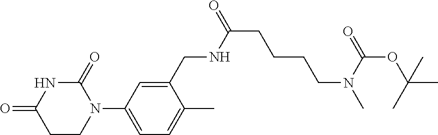 [SMILES: Cc1ccc(N2CCC(=O)NC2=O)cc1CNC(=O)CCCCN(C)C(=O)OC(C)(C)C]

HATU (CAS No. [148893-10-1], 324 mg, 0.85 mmol) was added to a stirred solution of 1-(5-(aminomethyl)-2-methylphenyl)dihydropyrimidine-2,4(1H,3H)-dione (250 mg, 0.72 mmol) and 5-((tert-butoxycarbonyl)(methyl)amino)pentanoic acid (CAS No. [124073-08-1], 200 mg, 0.86 mmol) followed by the addition of DIEA (CAS No. [7087-68-5], 186 mg, 1.44 mmol). The resulting solution was stirred at RT for 16 h. The RM was purified by reverse phase HPLC (0%-50% ACN in H₂O, 0.1% NH₄CO₃) to afford the title compound as a white solid. Method G: Rt=1.36 min; [M-BOC+H]⁺=347.

### Step 5: N-(5-(2,4-dioxotetrahydropyrimidin-1(2H)-yl)-2-methylbenzyl)-5-(methylamino)pentanamide

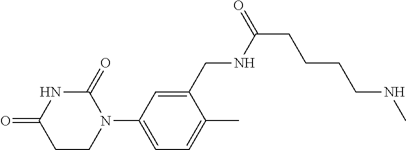 [SMILES: CNCCCCC(=O)NCc1cc(N2CCC(=O)NC2=O)ccc1C]

tert-Butyl (5-((5-(2,4-dioxotetrahydropyrimidin-1(2H)-yl)-2-methylbenzyl)amino)-5-oxopentyl)(methyl)carbamate (200 mg, 0.45 mmol) was dissolved in DCM (2 mL). TFA (6 mL) was added and the RM was stirred at RT for 16 h. The solution was concentrate to afford the title compound as a dark liquid. Method G: Rt=1.26 min; [M+H]⁺=347.

### Intermediate BB: 1-(4-iodophenyl)dihydropyrimidine-2,4(1H,3H)-dione

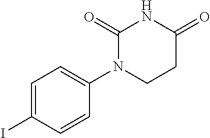 [SMILES: O=C1CCN(c2ccc(I)cc2)C(=O)N1]

### Step 1: 3-((4-iodophenyl)amino)propanoic acid

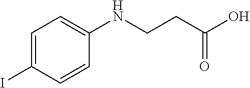 [SMILES: O=C(O)CCNc1ccc(I)cc1]

4-iodoaniline (4.0 g, 18.26 mmol) and acrylic acid (1.503 mL, 21.92 mmol) were dissolved in toluene (100 mL) and the reaction mixture was refluxed at 110° C. for 4 days. The reaction mixture was then cooled to room temp and concentrated. The crude solid was redissolved in a 1:1:1 solution of DMSO/water/ACN and purified via reverse phase chromatography on a Redisep® C18 column eluting with ACN in an aq. solution of TFA (1%) (from 10 to 80%) to afford the TFA salt of the title compound as a light brown solid (3.70 g, 9.14 mmol). Method XQ: RT=0.85 min; [M+H]⁺=292.1.

### Step 2: 1-(4-iodophenyl)dihydropyrimidine-2,4(1H,3H)-dione

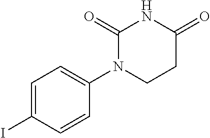 [SMILES: O=C1CCN(c2ccc(I)cc2)C(=O)N1]

3-((4-iodophenyl)amino)propanoic acid (3.7 g, 12.71 mmol) was dissolved in acetic acid (50 mL) and sodium cyanate (2.479 g, 38.1 mmol) was added. The reaction was heated at 90° C. for 18 h. The reaction mixture was cooled to RT, neutralized with 1N NaOH, and extracted with EtOAc (3×50 mL). The combined organics were washed with water (1×25 mL) and brine (1×25 mL), dried over magnesium sulfate, filtered, and concentrated. The crude residue was purified via FCC (0-15% MeOH/DCM) and further via reverse phase chromatography on a Redisep® C18 column eluting with ACN in an aq. solution of NH₄OH (0.1%) (from 10 to 75%) to afford the title compound (400 mg). Method XR: Rt=1.38 min; [M+H]⁺=317.0. ¹H NMR (400 MHz, DMSO-d6) S 10.41 (s, 1H), 7.81-7.65 (m, 2H), 7.23-7.10 (m, 2H), 3.78 (t, J=6.6 Hz, 2H), 2.70 (t, J=6.6 Hz, 2H).

### Intermediate CC: 3-((7-(3-chloropropoxy)quinazolin-4-yl)amino)-4-(dimethylamino)-N-methylbenzenesulfonamide

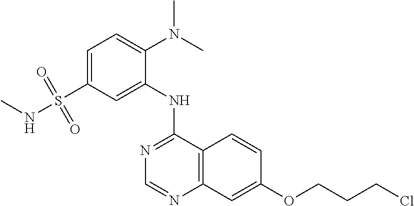 [SMILES: CNS(=O)(=O)c1ccc(N(C)C)c(Nc2ncnc3cc(OCCCCl)ccc23)c1]

This compound was prepared according to a procedure published in WO 2011/56740 A1; page 49, Example 16.

### Intermediate DD: tert-butyl 4-(prop-2-yn-1-yl)-1-oxa-4,9-diazaspiro[5.5]undecane-9-carboxylate

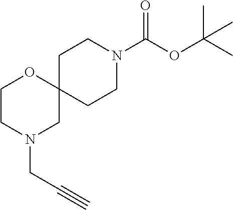 [SMILES: C#CCN1CCOC2(CCN(C(=O)OC(C)(C)C)CC2)C1]

To a solution of K₂CO₃ (415 mg, 3.00 mmol) in acetonitrile (10 mL) was added tert-butyl 1-oxa-4,9-diazaspiro[5.5]undecane-9-carboxylate (CAS No. [930785-40-3], 550 mg, 2.146 mmol) under argon. After 10 minutes, a solution of propargyl bromide in toluene (0.335 mL, 3.00 mmol) was added. The mixture was heated at reflux for 18 h, at which point, an additional 8 mg of K₂CO₃ and 10 mg of propargyl bromide was added to the reaction mixture. The reaction mixture was refluxed for an additional 24 hours, then cooled to room temperature and filtered to remove solids. The filtrate was concentrated and purified via Flash chromatography (0-100% EtOAc/Cyclohexane) to afford the title compound (594 mg, 2.018 mmol). Method LCMS1: Rt=0.95 min; [M+H]⁺=295.3.

### Intermediate EE: 1-(3-iodophenyl)dihydropyrimidine-2,4(1H,3H)-dione

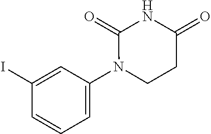 [SMILES: O=C1CCN(c2cccc(I)c2)C(=O)N1]

### Step 1: 3-((3-iodophenyl)amino)propanoic acid

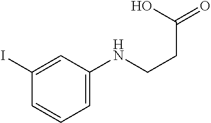 [SMILES: O=C(O)CCNc1cccc(I)c1]

To a solution of 3-iodoaniline (10 g, 45.66 mmol) in toluene (131 mL) was added acrylic acid (4.28 g, 59.36 mmol). The mixture was stirred at 115° C. for 48 h. The solvent was removed to obtain the title compound as an orange oil (15 g, crude). Method H: Rt=1.36 min; [M+H]⁺=291.9.

### Step 2: 1-(3-iodophenyl)dihydropyrimidine-2,4(1H,3H)-dione

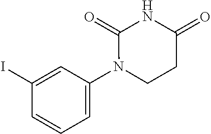 [SMILES: O=C1CCN(c2cccc(I)c2)C(=O)N1]

To a solution of 3-((3-iodophenyl)amino)propanoic acid (15 g, 51.5 mmol) in AcOH (125 mL) was added urea (9.284 g, 154.6 mmol). The mixture was stirred at 120° C. for 16 h. The solvent was removed and water (200 mL) was added. The mixture was filtered. The filter cake was washed with water (2×20 mL) and dried in vacuum. The solid was suspended in EtOAc (60 mL), triturated for 16 h at RT. The mixture was filtered. The filter cake was washed with EtOAc (2×5 mL) and dried to afford the title compound as a pale solid (7.9 g). Method E: Rt=1.43 min; [M+H]⁺=317.0.

### Intermediate FF: 4-(2,4-dioxotetrahydropyrimidin-1(2H)-yl)-3-methoxy-N-(4-(piperazin-1-yl)butyl)benzamide

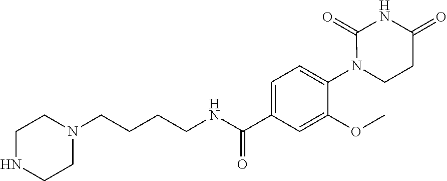 [SMILES: COc1cc(C(=O)NCCCCN2CCNCC2)ccc1N1CCC(=O)NC1=O]

### Step 1: tert-Butyl 4-(4-(4-(2,4-dioxotetrahydropyrimidin-1(2H)-yl)-3-methoxybenzamido)butyl)piperazine-1-carboxylate

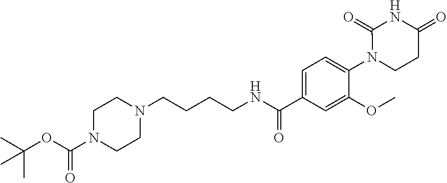 [SMILES: COc1cc(C(=O)NCCCCN2CCN(C(=O)OC(C)(C)C)CC2)ccc1N1CCC(=O)NC1=O]

A solution of 4-(2,4-dioxotetrahydropyrimidin-1(2H)-yl)-3-methoxybenzoic acid (ILB-81, 155 mg, 0.539 mmol), 4-(4-amino-butyl)-piperazine-1-carboxylic acid tert-butyl ester (CAS No. [745048-07-1], 146 mg, 0.539 mmol), HATU (293 mg, 0.755 mmol) and NMM (0.30 mL, 2.70 mmol) in DMF (5 mL) was stirred for 3 h at RT. The crude product was loaded on a Redisep® C18 column and eluted from (water+0.1% TFA)/ACN 98:2 to 1:9 to afford the title compound as a white powder (179 mg). Method LCMS1: Rt=0.59 min; [M+H]⁺=504.

### Step 2: 4-(2,4-dioxotetrahydropyrimidin-1(2H)-yl)-3-methoxy-N-(4-(piperazin-1-yl)butyl)benzamide

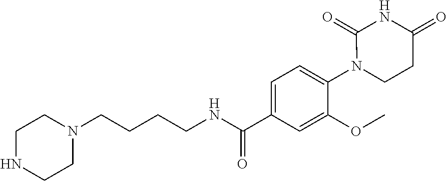 [SMILES: COc1cc(C(=O)NCCCCN2CCNCC2)ccc1N1CCC(=O)NC1=O]

A solution of tert-butyl 4-(4-(4-(2,4-dioxotetrahydropyrimidin-1(2H)-yl)-3-methoxybenzamido)butyl)piperazine-1-carboxylate (175 mg, 0.269 mmol) and HCl 4 N in dioxane (4 mL, 16 mmol) was stirred in methanol (2 mL) for 1.5 h at RT. The solvent was removed, the residue redissolved in ACN/H₂O and freeze dried to afford the title compound as a pale beige powder (131 mg). Method LCMS2: Rt=0.69 min; [M+H]⁺=404.

### Intermediate GG: 3-((2-(trimethylsilyl)ethoxy)methyl)dihydropyrimidine-2,4(1H,3H)-dione

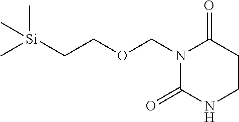 [SMILES: C[Si](C)(C)CCOCN1C(=O)CCNC1=O]

To a 100 mL round bottom flask were added dihydropyrimidine-2,4(1H,3H)-dione (2.7 g, 23.66 mmol) and anhydrous DMPU (35 mL). A solution of LiHMDS (1 M) in THF (25 mL, 25.00 mmol) was added under argon and the RM was vigorously stirred at 60° C. for 40 min. The RM was cooled to RT. SEM-Cl (5 mL, 28.20 mmol) was added and the RM was stirred at 60° C. for 18 h. The RM was diluted with sat. NaHCO₃ sol. and brine, extracted with EtOAc (×3). The organic phase was washed with water and brine, dried over MgSO₄ and concentrated. The residue was purified by chromatography on silica gel eluting with EtOAc in CHX (from 0% to 100%) and then with MeOH (from 0% to 20%) to afford the title compound as a slightly yellow oil (4.7 g). Method LCMS4: Rt=0.76 min; [M−H]⁻=243.

### Compound HH: 4-(2-(3-((2,4-dioxotetrahydropyrimidin-1(2H)-yl)methyl)-2-oxopyridin-1(2H)-yl)ethoxy)butanal

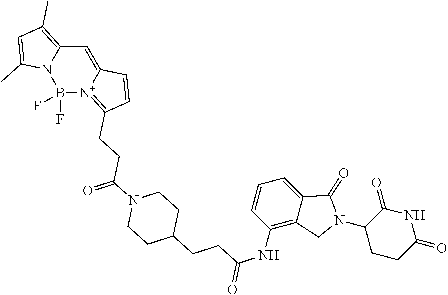

### Step 1: tert-butyl 4-(3-((2-(2,6-dioxopiperidin-3-yl)-1-oxoisoindolin-4-yl)amino)-3-oxopropyl)piperidine-1-carboxylate

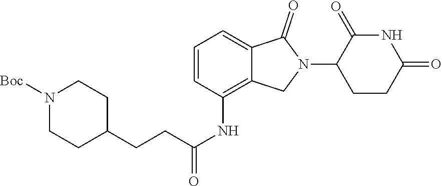 [SMILES: CC(C)(C)OC(=O)N1CCC(CCC(=O)Nc2cccc3c2CN(C2CCC(=O)NC2=O)C3=O)CC1]

In a MW vial were put lenalidomide (CAS No. [191732-72-6], 50 mg, 0.193 mmol), N-Boc-4-piperidinepropionic acid (CAS No. [154775-43-6], 65 mg, 0.212 mmol) and HATU (83 mg, 0.212 mmol) followed by 1.25 mL ACN and 0.5 mL DMF. DIPEA (0.1 mL, 0.579 mmol) was added before the vial was flushed with N₂ and capped. The pale yellow mixture was stirred for 21 h. The RM was concentrated and partitioned between EtOAc (5-6 mL) and buffer pH=4 (commercial solution, Fluka product number 33643, containing citric acid, sodium hydroxide and sodium chloride, 5 mL). Layers were separated and the aq. layer was extracted with EtOAc (5 mL) and the combined organic layers were dried over MgSO₄, filtered and concentrated under HV overnight to give a pale yellow resin. The crude product was purified by Redisep® ISCO—column 12 g SiO₂ with a DCM/iPrOH gradient to afford the title compound as white solid (87 mg). Method LCMS1: Rt=0.89 min; [M−H]⁺=499.1. ¹H NMR (400 MHz, DMSO-d6): 0.92-1.03 (m, 2H) 1.34-1.46 (m, 10H) 1.54 (q, J=7.09 Hz, 2H) 1.64 (br d, J=11.86 Hz, 2H) 1.96-2.06 (m, 1H) 2.30-2.40 (m, 3H) 2.55-2.75 (m, 3H) 2.83-2.97 (m, 1H) 3.91 (br d, J=12.10 Hz, 2H) 4.25-4.42 (m, 2H) 5.13 (dd, J=13.27, 5.07 Hz, 1H) 7.42-7.54 (m, 2H) 7.79 (dd, J=6.97, 1.47 Hz, 1H) 9.76 (s, 1H) 11.00 (s, 1H).

### Step 2: N-(2-(2,6-dioxopiperidin-3-yl)-1-oxoisoindolin-4-yl)-3-(piperidin-4-yl)propanamide

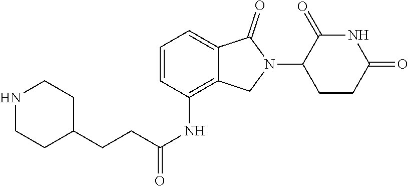 [SMILES: O=C1CCC(N2Cc3c(NC(=O)CCC4CCNCC4)cccc3C2=O)C(=O)N1]

tert-Butyl 4-(3-((2-(2,6-dioxopiperidin-3-yl)-1-oxoisoindolin-4-yl)amino)-3-oxopropyl)piperidine-1-carboxylate (265 mg, 0.532 mmol) and 3.98 mL of 4M HCl yielded a white suspension which was stirred at RT under N₂ atmosphere. After 1 hour, the RM was concentrated until dryness, and dried under HV pump. The solid residue was then co-evaporated with DCM (2×) to give a pale yellow powder which was dried at HV over night to yield the final product in 86% purity determined by NMR. The compound was used without further purification in the next reaction. Method LCMS1: Rt=0.43 min; [M−H]⁺=399.2.

### Step 3: tert-butyl 4-(3-((2-(2,6-dioxopiperidin-3-yl)-1-oxoisoindolin-4-yl)amino)-3-oxopropyl)piperidine-1-carboxylate

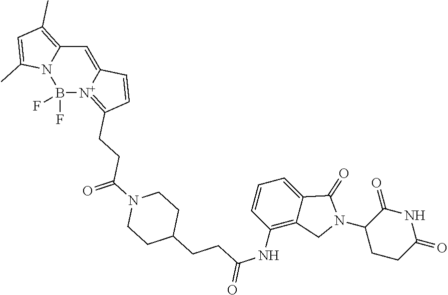

N-(2-(2,6-dioxopiperidin-3-yl)-1-oxoisoindolin-4-yl)-3-(piperidin-4-yl)propanamide (20 mg, 0.046 mmol) and BODIPY-FL propionic acid (13.43 mg, 0.046 mmol) have been dissolved in DMF (Volume: 0.5 mL) to give a fluorescence reddish solution [commercial, preparation see Krajcovicova et al., Chemistry—A European Journal, 24(19): 4957-4966 (2018)]. DIPEA (0.060 mL, 0.343 mmol) was added and the reaction was stirred in the dark and was monitored by UPLC/MS after 45 minutes. Trifluoroacetic acid (14.17 μL, 0.184 mmol) was added until the color changed to greenish. The crude product was submitted for R^{p} purification using the method XS (Sunfire C18 (5 μm, 30×100 mm), 40 mL/min, 29-49% over 16 min, total 21 min). Pure fractions were lyophilized overnight to afford the title compound as bright orange fluffy powder, which turns into fluorescent yellow upon solution in DMSO (27 mg). Method LCMS1: Rt=0.93 min; [M−H]⁺=673.4.

### Example 5: Intermediate Compound Synthesis

### ILB-1: 1-((1-allyl-2-oxo-1,2-dihydropyridin-3-yl)methyl)dihydropyrimidine-2,4(1H,3H)-dione

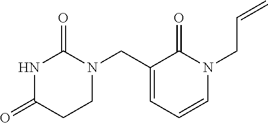 [SMILES: C=CCn1cccc(CN2CCC(=O)NC2=O)c1=O]

### Step 1: 3-(aminomethyl)pyridin-2(1H)-one

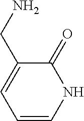

To a 1 L round bottom flask were added 2-oxo-1,2-dihydropyridine-3-carbonitrile (CAS No. [20577-27-9], 12 g, 100 mmol), Raney Ni (3 g), a solution of NH₃ (7 M) in MeOH (100 mL) and MeOH (150 mL). The reaction mixture was stirred under H₂ (1 atm) at RT for 48 h, filtered and the filtrate was concentrated, yielding a yellow oil (13.5 g), which was used for the next step without further purification. Method A: Rt=0.48 min; [M+H]⁺=125.

### Step 2: tert-butyl ((2-oxo-1,2-dihydropyridin-3-yl)methyl)carbamate

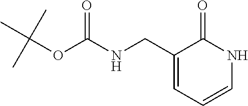 [SMILES: CC(C)(C)OC(=O)NCc1ccc[nH]c1=O]

To a 1 L round bottom flask were added 3-(aminomethyl)pyridin-2(1H)-one (13.5 g, 100 mmol), DIEA (25.8 g, 200 mmol), MeOH (200 mL), DCM (300 mL) and di-tert-butyl dicarbonate (21.8 g, 100 mmol). The reaction mixture was stirred at RT for 16 h, concentrated and the residue was purified by chromatography on silica gel eluting with MeOH in DCM from 0% to 8% to afford the title compound as an oil (10.0 g). Method B: Rt=1.61 min; [M+H]⁺=225.

### Step 3: tert-butyl ((1-allyl-2-oxo-1,2-dihydropyridin-3-yl)methyl)carbamate

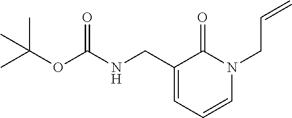 [SMILES: C=CCn1cccc(CNC(=O)OC(C)(C)C)c1=O]

To a 250 mL round bottom flask were added tert-butyl ((2-oxo-1,2-dihydropyridin-3-yl)methyl)carbamate (10.0 g, 45 mmol), K₂CO₃ (12.4 g, 90 mmol), DMF (80 mL) and 3-bromoprop-1-ene (CAS No. [106-95-6], 8.1 g, 67 mmol). The RM was stirred at RT for 16 h, filtered and the filtrate was poured into water (500 mL). The mixture was extracted with EtOAc (4×300 mL) and the combined organic phases were dried over Na₂SO₄, yielding the title compound as an oil (14.0 g). Method B: Rt=1.78 min; [M+H]⁺=265.

### Step 4: 1-allyl-3-(aminomethyl)pyridin-2(1H)-one

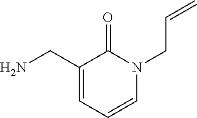

To a 1 L round bottom flask were added tert-butyl ((1-allyl-2-oxo-1,2-dihydropyridin-3-yl)methyl)carbamate (14.0 g), DCM (300 mL), and a solution of HCl (4 M) in 1,4-dioxane (50 mL). The reaction mixture was stirred at RT for 16 h, the solvents were removed and the residue was purified by reversed phase chromatography on a Biotage Agela C18 column (120 g, spherical 20-35 μm, 100 Å) eluting with ACN in aq. ammonium hydrogen carbonate (0.1%) from 5% to 40%, yielding the title compound as an oil (7.2 g). Method B: Rt=1.14 min; [M+H]⁺=165.

### Step 5: 3-(((1-allyl-2-oxo-1,2-dihydropyridin-3-yl)methyl)amino)propanoic acid

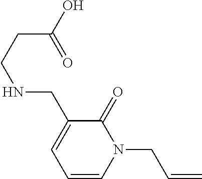 [SMILES: C=CCn1cccc(CNCCC(=O)O)c1=O]

To a 250 mL round bottom flask were added 1-allyl-3-(aminomethyl)pyridin-2(1H)-one (3.28 g, 20 mmol), acrylic acid (4.32 g, 60 mmol) and toluene (100 mL). The RM was stirred at 100° C. for 18 h, concentrated to afford the crude title compound used to the next step without further purification. Method C: Rt=0.34 min; [M+H]⁺=237.

### Step 6: 1-((1-allyl-2-oxo-1,2-dihydropyridin-3-yl)methyl)dihydropyrimidine-2,4(1H,3H)-dione

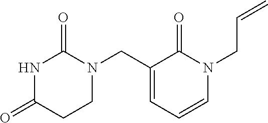 [SMILES: C=CCn1cccc(CN2CCC(=O)NC2=O)c1=O]

To a 250 mL round bottom flask were added 3-(((1-allyl-2-oxo-1,2-dihydropyridin-3-yl)methyl)amino)propanoic acid (8 g), urea (3.6 g, 60 mmol) and acetic acid (40 mL). The reaction mixture was stirred at 120° C. for 18 h, concentrated and the residue was purified by reversed phase chromatography on a Biotage Agela C18 column (120 g, spherical 20-35 μm, 100 Å) eluting with ACN in aq. ammonium hydrogen carbonate (0.1%) from 5% to 50%, yielding the title compound as a solid (3.4 g). Method B: Rt=1.40 min; [M+H]⁺=262.

### ILB-2: 2-(3-((2,4-Dioxotetrahydropyrimidin-1(2H)-yl)methyl)-2-oxopyridin-1(2H)-yl)acetaldehyde

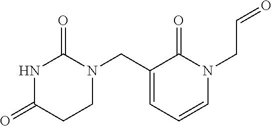 [SMILES: O=CCn1cccc(CN2CCC(=O)NC2=O)c1=O]

To a 250 mL round bottom flask were added 1-((1-allyl-2-oxo-1,2-dihydropyridin-3-yl)methyl)dihydropyrimidine-2,4(1H,3H)-dione (ILB-1, 3.9 g, 15 mmol), THF (120 mL), and a solution of OsO₄ (4%) in water (8 mL). The reaction mixture was stirred under nitrogen atmosphere at RT for 45 min. Solid NaIO₄ (9.6 g, 45 mmol) was added and the reaction mixture was stirred under nitrogen atmosphere at RT for 16 h. The mixture was filtered, the solvents were removed and the residue was purified by reversed phase chromatography on a Biotage Agela C18 column (120 g, spherical 20-35 μm, 100 Å) eluting with ACN in aq. ammonium hydrogen carbonate (0.1%) from 0% to 30%, yielding the title compound as a solid (3.6 g). Method D: Rt=0.42 min; [M+H]⁺=264

### ILB-3: 1-((1-(2-Hydroxyethyl)-2-oxo-1,2-dihydropyridin-3-yl)methyl)dihydropyrimidine-2,4(1H,3H)-dione

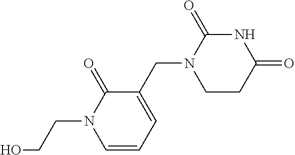 [SMILES: O=C1CCN(Cc2cccn(CCO)c2=O)C(=O)N1]

To a mixture of ILB-2 (80 mg, 0.30 mmol) in THF (3 mL) at 25° C. was added NaBH₄ (17 mg, 0.46 mmol) and the reaction mixture was stirred at 25° C. for 0.5 h. The RM was then cooled to 0° C. and water (1 mL) was added carefully. The solvent was removed in vacuo and the crude mixture was purified by flash chromatography on silica gel eluting with 0-10% MeOH in DCM to afford the desired product, 1-((1-(2-hydroxyethyl)-2-oxo-1,2-dihydropyridin-3-yl)methyl)dihydropyrimidine-2,4(1H,3H)-dione, as a white solid (52 mg). Method G: Rt=0.466; [M+H]⁺=266. ¹H NMR (500 MHz, DMSO) δ 10.16 (s, 1H), 7.55 (m, 1H), 7.28 (d, J=6.8 Hz, 1H), 6.20 (t, J=6.8 Hz, 1H), 4.88 (t, J=5.4 Hz, 1H), 4.27 (s, 2H), 3.96 (t, J=5.4 Hz, 2H), 3.62 (q, J=5.4 Hz, 2H), 3.42 (t, J=6.8 Hz, 2H), 2.57 (t, J=6.8 Hz, 2H).

### ILB-5: 4-(2-(3-((2,4-dioxotetrahydropyrimidin-1(2H)-yl)methyl)-2-oxopyridin-1(2H)-yl)ethoxy)butanal

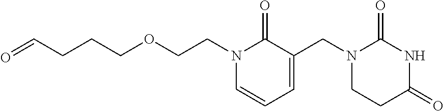 [SMILES: O=CCCCOCCn1cccc(CN2CCC(=O)NC2=O)c1=O]

### Step 1: 12,12-Dimethyl-1,11,11-triphenyl-2,5,10-trioxa-11-silatridecane

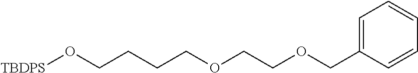

To a solution of 2-(benzyloxy)ethan-1-ol (CAS No. [622-08-2], commercially available, 9.91 g, 65.15 mmol) in THF (150 mL) was slowly added NaH (6.95 g, 173.73 mmol). The reaction mixture was warmed to 80° C. and stirred at this temperature for 1 h. After cooling to RT, (4-bromobutoxy)(tert-butyl)diphenylsilane (CAS No. [125010-58-4], Angew. Chem. Int. Ed. 54 (51): 15717-15720 (2015), 17 g, 43.43 mmol) was added dropwise. The reaction mixture solution was stirred at 80° C. for 16 h. The reaction mixture was slowly added to water (100 mL) and extracted with EtOAc (3×150 mL). The combined organic layers were washed with brine (2×60 mL), dried with Na₂SO₄ and concentrated in vacuo to obtain crude product. The crude mixture was purified by flash chromatography on silica gel eluting with petroleum ether and a 0-5% gradient of EtOAc yielding of desired product as colorless oil (7 g). Method G: Rf=2.88 min, [M+NH₄]⁺=480.3.

### Step 2: 2-(4-((tert-butyldiphenylsilyl)oxy)butoxy)ethan-1-ol

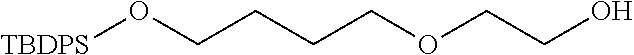

To a solution of 12,12-dimethyl-1,11,11-triphenyl-2,5,10-trioxa-11-silatridecane (10.8 g, 23.34 mmol) in EtOH/H₂O (100 mL/4 mL) was slowly added Pd/C (150 mg). The mixture was stirred at 40° C. for 16 h under H₂ atmosphere. The RM solution filtered and the solvent evaporated. The crude mixture was purified by chromatography on silica gel eluting with petroleum ether and a 0-50% gradient of EtOAc yielding the title compound as light yellow oil (8.1 g). Method G: Rf=2.310 min, [M+H]⁺=373.3.

### Step 3: 2-(4-((tert-butyldiphenylsilyl)oxy)butoxy)ethyl methanesulfonate

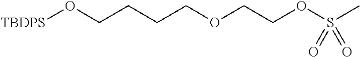

To a solution of 2-(4-((tert-butyldiphenylsilyl)oxy)butoxy)ethan-1-ol (8.1 g, 21.74 mmol) and TEA (6.60 g, 65.22 mmol) in DCM (50 mL) was slowly added MsCl (2.49 g, 21.74 mmol) dissolved in DCM (20 mL) via drop funnel at 0° C. After completed addition, the mixture was stirred for 3 h at 0° C. Water (20 mL) was added slowly and the mixture was extracted with DCM (3×100 mL). The combined organic layers were washed with brine (2×50 mL), dried with Na₂SO₄ and concentrated in vacuo to afford the crude title compound as a yellow oil (9.23 g) which was used directly in the next step without further purification. Method G: Rf=2.355 min, [M+NH₄]⁺=468.

### Step 4: tert-butyl(4-(2-iodoethoxy)butoxy)diphenylsilane

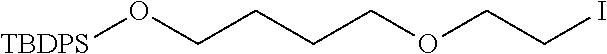

To a solution of 2-(4-((tert-butyldiphenylsilyl)oxy)butoxy)ethyl methanesulfonate (9.23 g, 20.48 mmol) in MeCN (100 mL) was added KI (34 g, 204.81 mmol) at RT and the mixture was stirred at 80° C. for 16 h. The mixture was poured into water (200 mL), extracted with EtOAc (2×100 mL), the combined organic layers were concentrated in vacuo to afford the crude title compound as a yellow oil (9.25 g), which was used directly in the next step without further purification. Method G: Rf=2.879 min, No mass observed (no ionization) purity: 100% (254 nm).

### Step 5: tert-butyl ((1-(2-(4-((tert-butyldiphenylsilyl)oxy)butoxy)ethyl)-2-oxo-1,2-dihydropyridin-3-yl)methyl)carbamate

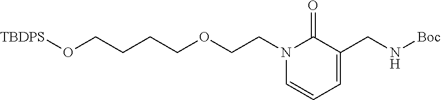

To a solution of tert-butyl ((2-oxo-1,2-dihydropyridin-3-yl)methyl)carbamate (ILB-1 Step 2, 4.3 g, 19.17 mmol) and tert-butyl(4-(2-iodoethoxy)butoxy)diphenylsilane (9.25 g, 19.17 mmol) in DMF (20 mL) was added K₂CO₃ (7.95 g, 33.44 mmol) and the mixture was stirred for 16 h at RT. The mixture was poured into water (200 mL), extracted with EtOAc (2×100 mL); the combined organic layers were washed with brine (5×5 mL) to remove the DMF. The organic layer was concentrated to give the crude product as yellow oil which was purified by flash chromatography on silica gel (petroleum ether, 10% to 60% ethyl acetate) to afford the title compound as light yellow oil (6.3 g). Method G: Rf=2.48 min, [M+H]⁺=579.

### Step 6: tert-butyl ((1-(2-(4-hydroxybutoxy)ethyl)-2-oxo-1,2-dihydropyridin-3-yl)methyl)carbamate

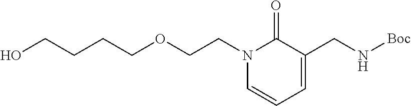 [SMILES: CC(C)(C)OC(=O)NCc1cccn(CCOCCCCO)c1=O]

TBAF (3.4 g, 13.06 mmol) was added to a solution of tert-butyl ((1-(2-(4-((tert-butyldiphenylsilyl)oxy)butoxy)ethyl)-2-oxo-1,2-dihydropyridin-3-yl)methyl)carbamate (6.3 g, 10.88 mmol) in 20 mL THF and the mixture was stirred at RT for 2 h. After evaporation of the solvent, the crude product was purified by flash chromatography on silica gel (10 to 100% EtOAc in petroleum ether followed by 0 to 10% MeOH in DCM) yielding the title compound as light yellow oil (3.3 g). Method G: Rf=1.56 min, [M+H]⁺=341.

### Step 7: 3-(aminomethyl)-1-(2-(4-hydroxybutoxy)ethyl)pyridin-2(1H)-one

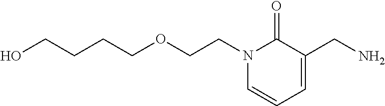 [SMILES: NCc1cccn(CCOCCCCO)c1=O]

To a solution of tert-butyl ((1-(2-(4-hydroxybutoxy)ethyl)-2-oxo-1,2-dihydropyridin-3-yl)methyl)carbamate (1.8 g, 5.29 mmol) in DCM/MeOH (30 mL/3 mL) at 0° C. was slowly added a solution of HCl in 1,4-dioxane (4M) (13.2 mL, 52.9 mmol). The RM was allowed to warm to RT and stirring was continued for 16 h. After removing the volatile components under reduced pressure, the residue was dissolved in H₂O/MeOH (8 mL/2 mL) and the pH was adjusted to 7.0 with aqueous Na₂CO₃ solution. The mixture solution was purified by reverse-phase chromatography using Method PB to afford the title compound as a white solid (800 mg). Method H: Rf=0.94 min, [M+H]⁺=241.3.

### Step 8: 3-(((1-(2-(4-hydroxybutoxy)ethyl)-2-oxo-1,2-dihydropyridin-3-yl)methyl)amino)propanoic acid

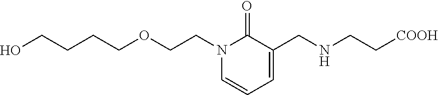 [SMILES: O=C(O)CCNCc1cccn(CCOCCCCO)c1=O]

A mixture of 3-(aminomethyl)-1-(2-(4-hydroxybutoxy)ethyl)pyridin-2(1H)-one (1.3 g, 5.42 mmol) and acrylic acid (780 mg, 10.8 mmol) in ACN (30 mL) was stirred at 80° C. for 4 h. The solvent was removed in vacuo to afford the title compound as a yellow oil (1.5 g) was used for the next step without further purification. Method H: Rf=0.89 min, [M+H]⁺=313.2.

### Step 9: 4-(2-(3-((2,4-dioxotetrahydropyrimidin-1(2H)-yl)methyl)-2-oxopyridin-1(2H)-yl)ethoxy)butyl acetate

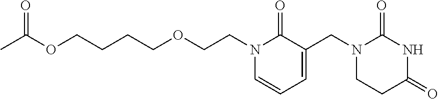 [SMILES: CC(=O)OCCCCOCCn1cccc(CN2CCC(=O)NC2=O)c1=O]

A mixture of 3-(((1-(2-(4-hydroxybutoxy)ethyl)-2-oxo-1,2-dihydropyridin-3-yl)methyl)amino)propanoic acid (1.5 g, crude, 4.8 mmol) and urea (1.15 g, 19.2 mmol) in HOAc (20 mL) was stirred at 100° C. for 16 h. The solvent was evaporated and the residue was purified by reverse-phase chromatography using Method PB to afford the title compound as a white solid (800 mg). Method XH: Rf=1.44 min, [M+H]⁺=380.0.

### Step 10: 1-((1-(2-(4-hydroxybutoxy)ethyl)-2-oxo-1,2-dihydropyridin-3-yl)methyl)dihydropyrimidine-2,4(1H,3H)-dione

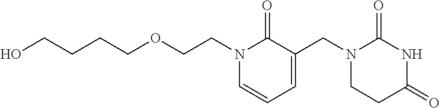 [SMILES: O=C1CCN(Cc2cccn(CCOCCCCO)c2=O)C(=O)N1]

To a solution of 4-(2-(3-((2,4-dioxotetrahydropyrimidin-1(2H)-yl)methyl)-2-oxopyridin-1(2H)-yl)ethoxy)butyl acetate (800 mg, 2.11 mmol) in 1,4-dioxane (10 mL) was added HCl (aq., 6 M, 20 mL). The reaction mixture was stirred at 90° C. for 30 min. The volatile components were removed under reduce pressure and the evaporation residue dissolved in water/MeCN (8 mL/2 mL) and the pH value was adjusted to 7.0 with aqueous Na₂CO₃ solution. The crude product solution was purified by reverse-phase chromatography using Method PB to afford the title compound as an off-white solid (600 mg). Method H: Rf=1.10 min, [M+H]⁺=338.3.

### Step 11: 4-(2-(3-((2,4-dioxotetrahydropyrimidin-1(2H)-yl)methyl)-2-oxopyridin-1(2H)-yl)ethoxy)butanal

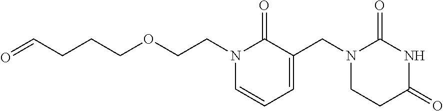 [SMILES: O=CCCCOCCn1cccc(CN2CCC(=O)NC2=O)c1=O]

A mixture of 1-((1-(2-(4-hydroxybutoxy)ethyl)-2-oxo-1,2-dihydropyridin-3-yl)methyl)dihydropyrimidine-2,4(1H,3H)-dione (200 mg, 0.593 mmol) and pyridinium chlorochromate (255 mg, 1.187 mmol) in DCM (10 mL) was stirred at RT for 4 h. The solids were removed by filtration and the filtrate was evaporated. The residue after evaporation was purified by reverse-phase chromatography using Method PB to afford the title compound as a light yellow solid (70 mg). Method G: Rf=1.50 min, [M+H]⁺=336.1.

### ILB-6: 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione

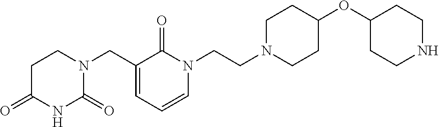 [SMILES: O=C1CCN(Cc2cccn(CCN3CCC(OC4CCNCC4)CC3)c2=O)C(=O)N1]

### Step 1: tert-butyl 4-(1-(2-(3-((2,4-dioxotetrahydropyrimidin-1(2H)-yl)methyl)-2-oxopyridin-1(2H)-yl)ethyl)piperidin-4-yloxy)piperidine-1-carboxylate

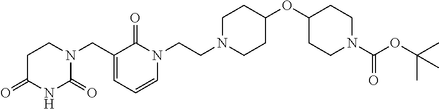 [SMILES: CC(C)(C)OC(=O)N1CCC(OC2CCN(CCn3cccc(CN4CCC(=O)NC4=O)c3=O)CC2)CC1]

To a 250 mL round bottom flask were added 2-(3-((2,4-dioxotetrahydropyrimidin-1(2H)-yl)methyl)-2-oxopyridin-1(2H)-yl)acetaldehyde (ILB-2, 3.6 g, 13.6 mmol), tert-butyl 4-(piperidin-4-yloxy)piperidine-1-carboxylate (CAS No. [845305-83-1], 3.86 g, 13.6 mmol), a solution of ZnCl₂ (1 M) in THF (20.4 mL, 20.4 mmol) and DMSO (40 mL). The RM was stirred at RT for 2 h, solid NaBH₃CN (2.57 g, 40.8 mmol) and MeOH (8 mL) were added, the reaction mixture was stirred at RT for 16 h, concentrated and purified by reverse phase chromatography on a Biotage Agela C18 column (120 g, spherical 20-35 μm, 100 Å) eluting with ACN in aq. ammonium hydrogen carbonate (0.1%) from 5% to 60%, yielding the title compound as a solid (2.8 g). Method A: Rt=1.81 min; [M+H]⁺=532.

### Step 2: 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione

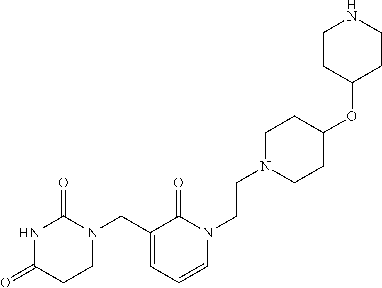 [SMILES: O=C1CCN(Cc2cccn(CCN3CCC(OC4CCNCC4)CC3)c2=O)C(=O)N1]

To a 250 mL round bottom flask were added tert-butyl 4-(1-(2-(3-((2,4-dioxotetrahydropyrimidin-1(2H)-yl)methyl)-2-oxopyridin-1(2H)-yl)ethyl)piperidin-4-yloxy)piperidine-1-carboxylate (2.8 g, 5.2 mmol), DCM (30 mL), and a solution of HCl (4 M) in 1,4-dioxane (10 mL). The reaction mixture was stirred at RT for 6 h, the mixture was concentrated and the residue purified by reversed phase chromatography on a Biotage Agela C18 column (120 g, spherical 20-35 μm, 100 Å) eluting with ACN in aq. ammonium hydrogen carbonate (0.1%) from 0% to 50%, yielding the title compound as a solid (1.8 g). Method B: Rt=1.36 min; [M+H]⁺=432.

### ILB-7: 1-(3-(2-Hydroxyethyl)benzyl)dihydropyrimidine-2,4(1H,3H)-dione

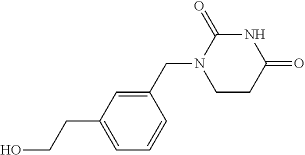 [SMILES: O=C1CCN(Cc2cccc(CCO)c2)C(=O)N1]

### Step 1: 2-(3-(Aminomethyl)phenyl)ethan-1-ol

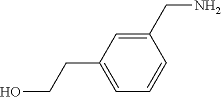 [SMILES: NCc1cccc(CCO)c1]

To a solution of ethyl 2-(3-(aminomethyl)phenyl)acetate (600 mg, 4.0 mmol) in THF (30 mL) at 0° C. was added dropwise a solution of LiAlH₄ (1M in THF, 8 mL, 8 mmol) and the mixture was stirred at RT for 4 h. An aqueous solution of Na₂SO₄ was added dropwise at 0° C. to quench the reaction and the mixture was filtered, then evaporated. The residue was purified by reverse phase chromatography eluting with ACN in an aq. solution of NH₄CO₃H (0.1%), providing the desired product, 2-(3-(aminomethyl)phenyl)ethan-1-ol (240 mg), as a yellow solid. LCMS Method XJ: Rt=1.45 min; [M+H]⁺=152.

### Step 2: 3-((2,4-Dioxotetrahydropyrimidin-1(2H)-yl)methyl)phenethyl acetate

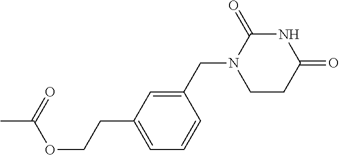 [SMILES: CC(=O)OCCc1cccc(CN2CCC(=O)NC2=O)c1]

A mixture of 2-(3-(aminomethyl)phenyl)ethan-1-ol (240 mg, 1.6 mmol) and acrylic acid (137 mg, 1.9 mmol) in toluene (10 mL) was heated at 100° C. for 16 h, then cooled to RT. The solvent was removed under vacuum to provide a yellow solid. Acetic acid (5 mL) was added, then urea (384 mg, 6.4 mmol). The reaction mixture was heated at 120° C. for 72 h, then cooled to RT and the acetic acid was removed under vacuum. Purification by reverse phase chromatography, eluting with ACN in an aq. solution of formic acid (0.1%), provided the desired product (170 mg) as a yellow solid. LCMS Method XJ: Rt=1.57 min; [M+H]⁺=291.

### Step 3: 1-(3-(2-Hydroxyethyl)benzyl)dihydropyrimidine-2,4(1H,3H)-dione

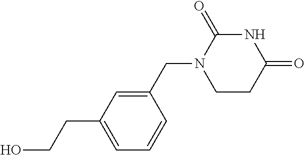 [SMILES: O=C1CCN(Cc2cccc(CCO)c2)C(=O)N1]

Aqueous hydrochloric acid (6M, 2 mL) was added to 3-((2,4-dioxotetrahydropyrimidin-1(2H)-yl)methyl)phenethyl acetate (170 mg, 0.6 mmol) in dioxane (4 mL). The reaction mixture was stirred at 80° C. for 1 h, then cooled to RT. The solvent was removed under vacuum and the residue was purified by reverse phase chromatography (Method PB) eluting with ACN in an aq. solution of NH₄CO₃H to provide the title compound (50 mg) as a white solid. LCMS Method XE: Rt=1.27 min; [M+H]⁺=249. ¹H NMR (500 MHz, DMSO) δ 7.25 (t, J=7.9 Hz, 1H), 7.13-7.09 (m, 3H), 4.63 (br. s, 1H), 4.49 (s, 2H), 3.60-3.57 (m, 2H), 3.27 (t, J=6.8 Hz, 2H), 2.71 (t, J=7 Hz, 2H), 2.53 (t, J=6.8 Hz, 2H).

### ILB-8: 2-((2,4-Dioxotetrahydropyrimidin-1(2H)-yl)methyl)isonicotinic acid

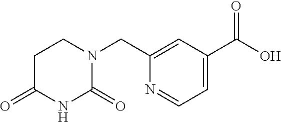 [SMILES: O=C1CCN(Cc2cc(C(=O)O)ccn2)C(=O)N1]

### Step 1: 3-(((4-(Methoxycarbonyl)pyridin-2-yl)methyl)amino)propanoic acid

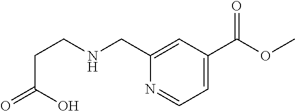 [SMILES: COC(=O)c1ccnc(CNCCC(=O)O)c1]

To a mixture of methyl 2-cyanoisonicotinate (7 g, 43.2 mmol) in MeOH (100 mL) was added Pd/C (500 mg) and conc. HCl (5 mL). The mixture was then stirred at 30° C. for 2 hours under a H₂ (15 psi) atmosphere. The mixture was filtered to remove Pd/C, then the filtrate was concentrated under reduced pressure to give methyl 2-(aminomethyl)isonicotinate (7 g, crude) as a yellow solid. To this material (7 g, 42.1 mmol), which was used without further purification, was added MeCN (35 mL), water (7 mL) and finally acrylic acid (3.96 g, 54.7 mmol). The mixture was stirred at 80° C. for 16 hrs, then the mixture was concentrated under vacuum to dryness. The solid was purified by flash column chromatography (ISCO®; 40 g SepaFlash® Silica Flash Column, Eluent of 0-100% DCM/MeOH) to afford the title compound 3-(((4-(methoxycarbonyl)pyridin-2-yl)methyl)amino)propanoic acid as yellow oil (5 g, 92% purity). Method LCMS10: Rt=0.26 min; [M+H]⁺=239.0.

### Step 2: 2-(((2-Carboxyethyl)amino)methyl)isonicotinic acid

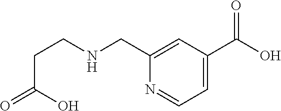 [SMILES: O=C(O)CCNCc1cc(C(=O)O)ccn1]

To a solution of 3-(((4-(methoxycarbonyl)pyridin-2-yl)methyl)amino)propanoic acid (2 g, 8.39 mmol) in MeOH (5 mL) was added THF (5 mL), H₂O (5 mL) and LiOH (1.01 g, 42 mmol). The mixture was stirred at 25° C. for 12 hours. The mixture was then concentrated to afford the crude 2-(((2-carboxyethyl)amino)methyl)isonicotinic acid (2 g) as a yellow solid. Method LCMS10: Rt=0.13 min; [M+H]⁺=225.0.

### Step 3: 2-((2,4-Dioxotetrahydropyrimidin-1(2H)-yl)methyl)isonicotinic acid

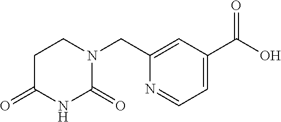 [SMILES: O=C1CCN(Cc2cc(C(=O)O)ccn2)C(=O)N1]

To a solution of 2-(((2-carboxyethyl)amino)methyl)isonicotinic acid (2 g, 8.92 mmol) in HOAc (20 mL) was added urea (1.61 g, 26.8 mmol) at 25° C. The reaction mixture was then heated with stirring at 100° C. for 12 hours. After cooling to RT, the reaction mixture was concentrated in vacuum to remove the HOAc to afford an oil (2.5 g, crude). The oil was purified by flash silica gel chromatography (ISCO®; 10 g SepaFlash® Silica Flash Column, Eluent of 0-100% DCM/MeOH) to afford, after lyophilization, a yellow solid (500 mg, 91% purity). This product was further purified by Prep-HPLC (Method PA2) to afford the title compound, 2-((2,4-dioxotetrahydropyrimidin-1(2H)-yl)methyl)isonicotinic acid which was obtained as a white solid (156 mg, 0.6 mmol, 97.8% purity). Method LCMS11: Rt=2.66 min; [M+H]+=250.1. ¹H NMR (400 MHz, DMSO) δ 10.25 (s, 1H), 8.75-8.69 (m, 1H), 7.74-7.70 (m, 2H), 4.71 (s, 2H), 3.46 (t, J=6.8 Hz, 2H), 2.59 (t, J=6.8 Hz, 2H).

### ILB-9: 3-((2,4-Dioxotetrahydropyrimidin-1(2H)-yl)methyl)-4-methoxybenzoic acid

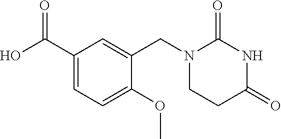 [SMILES: COc1ccc(C(=O)O)cc1CN1CCC(=O)NC1=O]

### Step 1: 3-((2-Methoxy-5-(methoxycarbonyl)benzyl)amino)propanoic acid

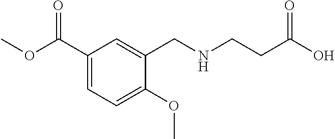 [SMILES: COC(=O)c1ccc(OC)c(CNCCC(=O)O)c1]

A mixture of methyl 3-(aminomethyl)-4-methoxybenzoate (CAS [771579-95-4], 1.90 g, 9.73 mmol) and acrylic acid (2.004 mL, 29.2 mmol) in toluene (48.7 mL) was stirred at 100° C. overnight. The RM was concentrated to dryness to afford the title compound as a yellow resin (3.75 g). Method LCMS7: Rt=0.41 min; [M+H]⁺=268.

### Step 2: Methyl 3-((2,4-dioxotetrahydropyrimidin-1(2H)-yl)methyl)-4-methoxybenzoate

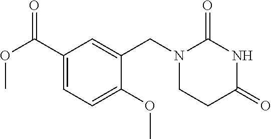 [SMILES: COC(=O)c1ccc(OC)c(CN2CCC(=O)NC2=O)c1]

Under argon, a mixture of 3-((2-methoxy-5-(methoxycarbonyl)benzyl)amino)propanoic acid (3.7 g, 13.84 mmol) and urea (1.663 g, 27.7 mmol) in acetic acid (18.5 mL) was stirred at 120° C. overnight. The mixture was cooled to RT, then mixed with ca. 100 mL ice and 20 mL conc. HCl. The opaque beige mixture was left to stir for 30 min, then stored in the fridge overnight. The cooled mixture was filtered, the residue washed with a little water, then Et₂O, then dried under high vacuum to afford 1.82 g of the desired product as an off-white solid. Method LCMS7: Rt=0.69 min; [M+H]⁺=293

### Step 3: 3-((1-(2-Carboxyethyl)ureido)methyl)-4-methoxybenzoic acid

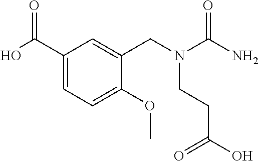 [SMILES: COc1ccc(C(=O)O)cc1CN(CCC(=O)O)C(N)=O]

A solution of lithium hydroxide monohydrate (CAS [1310-66-3], 2.58 g, 61.6 mmol) in water (30.8 mL) was added to a mixture of methyl 3-((2,4-dioxotetrahydropyrimidin-1(2H)-yl)methyl)-4-methoxybenzoate (1.80 g, 6.16 mmol) in THF (30.8 mL). The mixture was stirred for 2.5 h at RT. The THF was then removed under reduced pressure and the aqueous layer was washed with DCM, then acidified with 1N HCl to ca. pH3 (a white precipitate emerged on standing for few minutes). The mixture was then sonicated and filtered. The residue washed with water, then Et₂O, then dried to afford 1.69 g of the product as an off-white solid. Method LCMS7: Rt=0.54 min; [M+H]⁺=297

### Step 4: 3-((2,4-Dioxotetrahydropyrimidin-1(2H)-yl)methyl)-4-methoxybenzoic acid

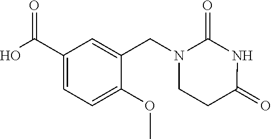 [SMILES: COc1ccc(C(=O)O)cc1CN1CCC(=O)NC1=O]

A mixture of 3-((1-(2-carboxyethyl)ureido)methyl)-4-methoxybenzoic acid (1.52 g, 5.13 mmol) in conc. HCl (15.6 mL, 513 mmol) was stirred for 1 hour at 100° C. The mixture was then cooled to RT and diluted with ice water. The precipitate was filtered off, washed with water and Et₂O, then dried to afford 1.23 g of the desired product as white solid. Method LCMS7: Rt=0.60 min; [M+H]⁺=279

### ILB-10: 3-((2,4-Dioxo-3-((2-(trimethylsilyl)ethoxy)methyl)tetrahydropyrimidin-1(2H)-yl)methyl)benzaldehyde

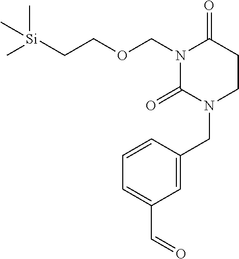 [SMILES: C[Si](C)(C)CCOCN1C(=O)CCN(Cc2cccc(C=O)c2)C1=O]

### Step 1: 3-((2-(trimethylsilyl)ethoxy)methyl)dihydropyrimidine-2,4(1H,3H)-dione

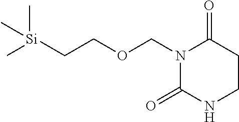 [SMILES: C[Si](C)(C)CCOCN1C(=O)CCNC1=O]

To a 100 mL round bottom flask were added under an argon atmosphere dihydropyrimidine-2,4(1H,3H)-dione (2.7 g, 23.66 mmol) and DMPU (35 mL). A solution of LiHMDS (1M) in THF (25 mL, 25.00 mmol) was added and the RM was vigorously stirred at 60° C. for 40 min. The RM was cooled to RT, 2-(trimethylsilyl)ethoxymethyl chloride (5 mL, 28.20 mmol) was added and the RM was stirred at 60° C. for 18 h. The RM was diluted with a sat. solution of NaHCO₃ and brine, the mixture was extracted with EtOAc, the combined organic phases were washed with water and brine, dried over MgSO₄ and the residue was purified by chromatography on silica gel eluting with EtOAc (from 0% to 100%) in CHX followed by MeOH (from 0% to 20%) in EtOAc, yielding the title compound as an oil (4.7 g). Method LCMS4: Rt=0.76 min; [M−H]⁺243.

### Step 2: 3-((2,4-dioxo-3-((2-(trimethylsilyl)ethoxy)methyl)tetrahydropyrimidin-1(2H)-yl)methyl)benzaldehyde

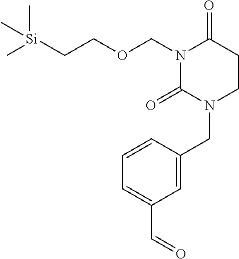 [SMILES: C[Si](C)(C)CCOCN1C(=O)CCN(Cc2cccc(C=O)c2)C1=O]

To a 10 mL round bottom flask were added under an argon atmosphere 3-((2-(trimethylsilyl)ethoxy)methyl)dihydropyrimidine-2,4(1H,3H)-dione (150 mg, 0.602 mmol) and DMF (3 mL). The mixture was cooled to 0° C., solid NaH (60% dispersion in mineral oil, 16 mg, 25.0 mmol) was added and the mixture was stirred at RT for 10 min. 3-(Bromomethyl)-benzaldehyde (132 mg, 0.632 mmol) was added and the RM was stirred at RT for 2 h. An aq. sat. solution of NH₄Cl and water were added, the aq. phase was extracted with EtOAc, and the combined organic phases were washed with brine, dried over MgSO₄ and the residue was purified by reversed phase chromatography on a RediSep® Gold HP C18 column (15.5 g) eluting with ACN (from 1% to 100%) in an aq. solution of NH₄HCO₃ (0.1%), yielding the title compound as an oil (62 mg). Method LCMS1: Rt=1.06 min; [M+H]⁺=380.

### ILB-12: 1-(2-chloro-4-hydroxyphenyl)dihydropyrimidine-2,4(1H,3H)-dione

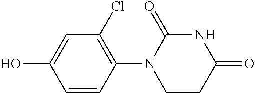 [SMILES: O=C1CCN(c2ccc(O)cc2Cl)C(=O)N1]

### Step 1: 3-((2-chloro-4-methoxyphenyl)amino)propanoic acid, 3,3′-((2-chloro-4-methoxyphenyl)azanediyl)dipropanoic acid

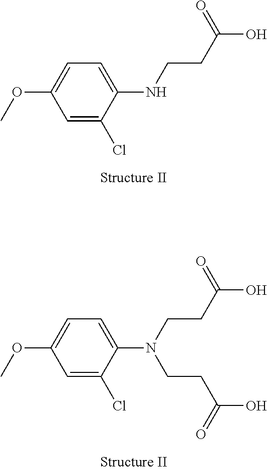 [SMILES: COc1ccc(NCCC(=O)O)c(Cl)c1 ; COc1ccc(N(CCC(=O)O)CCC(=O)O)c(Cl)c1]

A mixture of 4-methoxy-2-methylaniline (4.82 g, 30.6 mmol) and acrylic acid (8.40 mL, 122 mmol) in Toluene (Volume: 10 mL) was heated for 1 h at 100° C. After 1.5 h, the RM was evaporated to dryness to obtain 7.02 g of a mixture of Structure I and II as a black resin. UPLC-MS showed 47% structure I/19% structure II. Method LCMS1 for structure I: Rt=0.78 min; [M+H]⁺=230.1. Method LCMS1 for structure II: Rt=0.84 min; [M+H]⁺=302.1.

### Step 2: 1-(2-chloro-4-methoxyphenyl)dihydropyrimidine-2,4(1H,3H)-dione

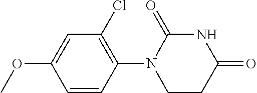 [SMILES: COc1ccc(N2CCC(=O)NC2=O)c(Cl)c1]

To a mixture of 3-((2-chloro-4-methoxyphenyl)amino)propanoic acid, 3,3′-((2-chloro-4-methoxyphenyl)azanediyl)dipropanoic acid (7.02 g, 30.6 mmol) in toluene (35 mL, ratio: 1.0) /acetic acid (35.0 mL, ratio: 1.0) was added urea (9.18 g, 153 mmol). The RM was heated overnight at 120° C. The RM was evaporated to dryness. The greasy residue was poured into 300 mL ice and stirred until reaching room temperature. The formed precipitate was filtered off and washed with water. The filter cake was washed with diisopropyl ether before drying overnight in vacuo at 50° C. to afford the title compound as a violet solid (5.11 g). Method LCMS1: Rt=0.66 min; [2M+H]⁺=509.2.

### Step 3: 1-(2-chloro-4-hydroxyphenyl)dihydropyrimidine-2,4(1H,3H)-dione

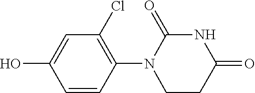 [SMILES: O=C1CCN(c2ccc(O)cc2Cl)C(=O)N1]

To a mixture of 1-(2-chloro-4-methoxyphenyl)dihydropyrimidine-2,4(1H,3H)-dione (2.29 g, 8.27 mmol) in DCM (40 mL) was added dropwise BBr₃ 1 M in CH₂Cl₂ (23.16 mL, 23.16 mmol) at RT. The RM was stirred at room temperature for 1.5 h. The RM was evaporated and absorbed on silica gel and purified by flash chromatography on a Silica flash column 24 g eluting with DCM/MeOH to afford the title compound (1.69 g). Method LCMS1: Rt=0.47 min; [M−H]⁺=239.1.

### ILB-13: 1-(4-hydroxy-2-methylphenyl)dihydropyrimidine-2,4(1H,3H)-dione

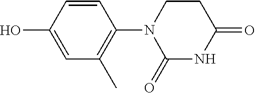 [SMILES: Cc1cc(O)ccc1N1CCC(=O)NC1=O]

### Step 1: 3-((4-Methoxy-2-methylphenyl)amino)propanoic acid, 3,3′-((4-methoxy-2-methylphenyl)azanediyl)dipropanoic acid

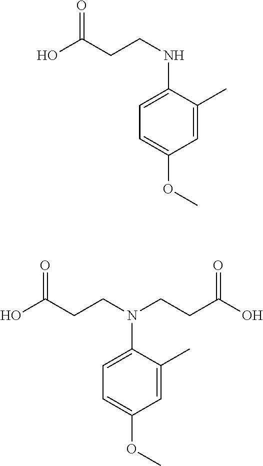 [SMILES: COc1ccc(NCCC(=O)O)c(C)c1 ; COc1ccc(N(CCC(=O)O)CCC(=O)O)c(C)c1]

A mixture of 4-methoxy-2-methylaniline (4.82 g, 35.1 mmol) and acrylic acid (9.65 mL, 141 mmol) in toluene (10 mL) was heated for 1.5 h at 100° C. The RM was evaporated to dryness to obtain a black resin. The black resin was used directly in 1-(4-methoxy-2-methylphenyl)dihydropyrimidine-2,4(1H,3H)-dione. Method LCMS1: Rt=0.53 min; [M+H]⁺=210.1 structure I. Method LCMS1: Rt=0.49 min; [M+H]⁺=282.2 structure II.

### Step 2: 1-(4-methoxy-2-methylphenyl)dihydropyrimidine-2,4(1H,3H)-dione

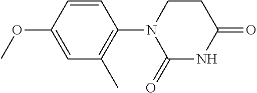 [SMILES: COc1ccc(N2CCC(=O)NC2=O)c(C)c1]

To a mixture of 3-((4-methoxy-2-methylphenyl)amino)propanoic acid (7.34 g, 35.1 mmol) in toluene (35 mL, ratio: 1.0)/acetic acid (35.0 mL, ratio: 1.0) was added urea (10.54 g, 176 mmol). The RM was heated overnight at 120° C. The RM was evaporated to dryness. The greasy residue was poured into 300 mL ice and stirred until reaching room temperature. The formed precipitate was filtered off and washed well with water. The cake was taken up in diisopropyl ether (soluble in acetonitrile) and filtered before drying overnight in vacuo at 50° C. to afford the tittle compound as a violet solid (4.34 g). Method LCMS1: Rt=0.61 min; [M+H]⁺=235.1.

### Step 3: 1-(4-hydroxy-2-methylphenyl)dihydropyrimidine-2,4(1H,3H)-dione

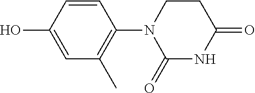 [SMILES: Cc1cc(O)ccc1N1CCC(=O)NC1=O]

To a mixture of 1-(4-methoxy-2-methylphenyl)dihydropyrimidine-2,4(1H,3H)-dione (2.5 g, 10.03 mmol) in DCM (30 mL) was added dropwise BBr₃ 1 M in CH₂Cl₂ (27.1 mL, 27.1 mmol) at RT. The RM was stirred at RT for 2 h. The RM was evaporated to dryness. The residue was used without purification in 1-(4-hydroxy-2-methylphenyl)dihydropyrimidine-2,4(1H,3H)-dione. Method LCMS1: Rt=0.43 min; [M+H]⁺=221.1.

### ILB-14: 2-(4-(2,4-Dioxotetrahydropyrimidin-1(2H)-yl)phenoxy)acetic acid

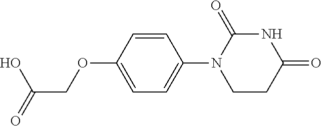 [SMILES: O=C(O)COc1ccc(N2CCC(=O)NC2=O)cc1]

### Step 1: Methyl 2-(4-((tert-butoxycarbonyl)amino)phenoxy)acetate

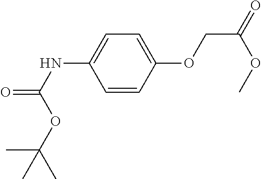 [SMILES: COC(=O)COc1ccc(NC(=O)OC(C)(C)C)cc1]

To a solution of tert-butyl (4-hydroxyphenyl)carbamate (7 g, 31.8 mmol) in acetone (75 mL) was added cesium carbonate (11.4 g, 35 mmol) and potassium iodide (50 mg, 0.301 mmol). Methyl bromoacetate (3 mL, 32.6 mmol) was added and the RM was stirred at reflux for 4 h. The RM was cooled to RT, filtered and the filtrate was concentrated to dryness. The residue was dissolved in EtOAc and washed with sat. aq. NaHCO₃ sol., dried over MgSO₄, and concentrated to dryness. The residue was purified by chromatography on silica gel eluting with EtOAc in CHX (from 10% to 25%) yielding the title compound as a white solid (8.83 g). Method LCMS1: Rt=0.97 min; [M+H]⁺=282.2.

### Step 2: Methyl 2-(4-aminophenoxy)acetate

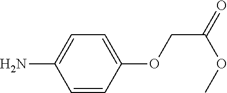

To a solution of methyl 2-(4-((tert-butoxycarbonyl)amino)phenoxy)acetate (8.83 g, 31.4 mmol) in 1,4-dioxane (30 mL) was added TFA (30 mL). The RM was stirred at RT overnight. The RM was concentrated to dryness and the residue dissolved in DCM. The organic phase was washed with sat. aq. NaHCO₃ sol., dried over MgSO₄, and concentrated to dryness to afford the title compound as an oil (5.35 g). Method LCMS1: Rt=0.37 min; [M+H]⁺=182.1.

### Step 3: 3,3′-((4-(2-Methoxy-2-oxoethoxy)phenyl)azanediyl)dipropanoic acid

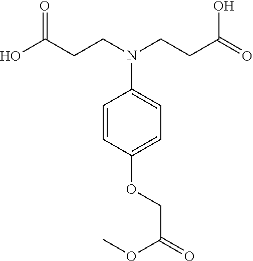 [SMILES: COC(=O)COc1ccc(N(CCC(=O)O)CCC(=O)O)cc1]

To a solution of methyl 2-(4-aminophenoxy)acetate (5347 mg, 25.7 mmol) in water (5 mL) at RT was added acrylic acid (11 mL, 160 mmol). The RM was stirred at 70° C. for 90 min. The RM was cooled to RT and adsorbed onto silica gel. The crude material was purified by chromatography on silica gel eluting with iPrOH in DCM (from 0% to 10%) yielding the title compound as a grey solid (8.24 g). Method LCMS1: Rt=0.47 min; [M+H]⁺=326.2.

### Step 4: 2-(4-(2,4-dioxotetrahydropyrimidin-1(2H)-yl)phenoxy)acetic acid

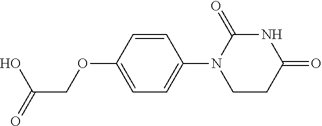 [SMILES: O=C(O)COc1ccc(N2CCC(=O)NC2=O)cc1]

A suspension of 3,3′-((4-(2-methoxy-2-oxoethoxy)phenyl)azanediyl)dipropanoic acid (8243 mg, 25.09 mmol) and urea (2260 mg, 37.6 mmol) in AcOH (60 mL) was stirred at 120° C. overnight. The RM was cooled to 0° C. and filtered to afford the title compound as an off-white solid (4.93 g). Method LCMS2: Rt=0.75 min; [M+H]⁺=265.2.

### ILB-16: 2-(3-(2,4-Dioxotetrahydropyrimidin-1(2H)-yl)phenoxy)acetic acid

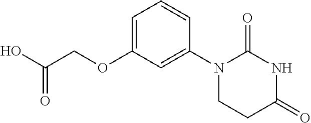 [SMILES: O=C(O)COc1cccc(N2CCC(=O)NC2=O)c1]

### Step 1: Methyl 2-(3-nitrophenoxy)acetate

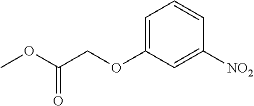

To a mixture of 3-nitrophenol (3.0 g, 21.57 mmol) and K₂CO₃ (4.0 g, 28.9 mmol) in acetone (10 mL) was added methyl bromoacetate (2.58 mL, 28.1 mmol) dropwise at RT. The mixture was stirred at 70° C. for 6 h. The RM was cooled to RT and diluted with water (40 mL). The resulting mixture was filtered and the filter cake was dried under HV, yielding the title compound as a slightly orange powder (4323 mg). ¹H NMR (400 MHz, DMSO-d₆) δ 7.85 (m, 1H), 7.73 (m, 1H), 7.59 (m, 1H), 7.45 (m, 1H), 4.99 (s, 2H), 3.72 (s, 3H).

### Step 2: Methyl 2-(3-aminophenoxy)acetate

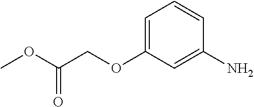

To a solution of methyl 2-(3-nitrophenoxy)acetate (1000 mg, 4.74 mmol) in MeOH (30 mL) at RT under argon was added palladium 10% on carbon (150 mg, 1.41 mmol). The flask was purged twice with argon and replaced three times by hydrogen gas taken from a balloon. The RM was stirred at RT for 22 h. The RM was diluted with MeOH (25 mL), filtered through Hyflow Super Cel®, and rinsed with MeOH (2×20 mL). The filtrate was concentrated. The residue was purified by filtration over silica gel 60 (230-400 mesh) using DCM/2% MeOH as eluent (3×50 mL), yielding the title compound as a brown oil (875 mg). Method LCMS1: Rt=0.57 min; [M+H]⁺=182.0.

### Step 3: 3,3′-((3-(2-Methoxy-2-oxoethoxy)phenyl)azanediyl)dipropionic acid

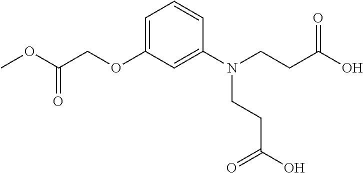 [SMILES: COC(=O)COc1cccc(N(CCC(=O)O)CCC(=O)O)c1]

To a mixture of methyl 2-(3-aminophenoxy)acetate (800 mg, 4.42 mmol) in H₂O (0.5 mL) was added acrylic acid (1.740 mL, 27.8 mmol) at RT. The RM was stirred at 70° C. under argon for 1.5 h. The residue was purified by chromatography on silica gel eluting with iPrOH in DCM (from 0.4% to 20%) yielding the title compound (1.30 g). Method LCMS1: Rt=0.66 min; [M+H]⁺=326.1.

### Step 4: 2-(3-(2,4-Dioxotetrahydropyrimidin-1(2H)-yl)phenoxy)acetic acid

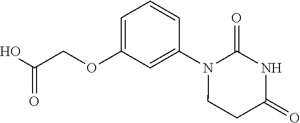 [SMILES: O=C(O)COc1cccc(N2CCC(=O)NC2=O)c1]

A mixture of 3,3′-((3-(2-methoxy-2-oxoethoxy)phenyl)azanediyl)dipropionic acid (1.30 g, 4.00 mmol) and urea (0.360 g, 5.99 mmol) in AcOH (8 mL) was stirred at 120° C. under argon overnight. A 10% aq. HCl sol. (20 mL) was added and the RM was refluxed for 1 h. The RM was cooled to RT and evaporated to dryness. The remaining solid was suspended in 10% aq. HCl sol, cooled to 0° C. and filtered yielding the title compound as a beige solid (575 mg). Method LCMS1: Rt=0.43 min; [M+H]⁺=265.1.

### ILB-17: 2-(4-Chloro-3-(2,4-dioxotetrahydropyrimidin-1(2H)-yl)phenoxy)acetic acid

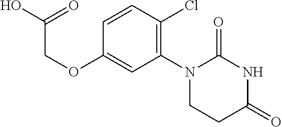 [SMILES: O=C(O)COc1ccc(Cl)c(N2CCC(=O)NC2=O)c1]

### Step 1: 1-(2-Chloro-5-hydroxyphenyl)dihydropyrimidine-2,4(1H,3H)-dione

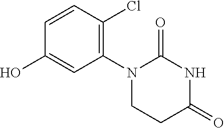 [SMILES: O=C1CCN(c2cc(O)ccc2Cl)C(=O)N1]

To a mixture of 3-amino-4-chlorophenol (1436 mg, 10.00 mmol) in H₂O (1 mL) was added acrylic acid (2.057 mL, 30.00 mmol) at RT. The RM was stirred at 70° C. under argon for 5.5 h. The RM was cooled to RT and dried. The residue was diluted in AcOH (15 mL). Urea (901 mg, 15.00 mmol) was added and the RM was stirred at 130° C. under argon overnight. Urea (500 mg, 8.33 mmol) was added and the RM was stirred at 130° C. under argon for 4.5 h. Urea (1000 mg, 16.65 mmol) was added and the RM was stirred at 130° C. under argon overnight. The RM was cooled to RT. 10% aq. HCl (20 mL) was added and the RM was refluxed for 30 min. The RM was cooled to RT and partially evaporated. EtOH (20 mL) was added and the mixture was cooled to 0° C. for 20 min and filtered. The filtrate was partially evaporated, adsorbed on Isolute® and purified by chromatography on silica gel eluting with EtOAc in CHX (from 80% to 100%). Fractions containing target compound were combined, evaporated, and recrystallized from hot acetone yielding the title compound as a white powder (1.09 g). Method LCMS1: Rt=0.53 min; [M+H]⁺=241.1.

### Step 2: Methyl 2-(4-chloro-3-(2,4-dioxotetrahydropyrimidin-1(2H)-yl)phenoxy)acetate

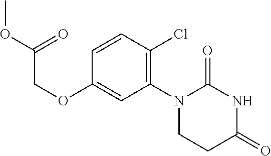 [SMILES: COC(=O)COc1ccc(Cl)c(N2CCC(=O)NC2=O)c1]

To a solution of 1-(2-chloro-5-hydroxyphenyl)dihydropyrimidine-2,4(1H,3H)-dione (353 mg, 1.467 mmol) and cesium carbonate (478 mg, 1.467 mmol) in DMF (15 mL) was added methyl 2-bromoacetate (0.135 mL, 1.467 mmol) at RT. The RM was stirred at RT overnight, concentrated, adsorbed on Isolute® and purified by chromatography on silica gel eluting with EtOAc in hexane (from 20% to 100%) and then with AcOH (from 0% to 1%), yielding the title compound as a white powder (400 mg). Method LCMS1: Rt=0.97 min; [M+H]⁺=313.1.

### Step 3: 2-(4-Chloro-3-(2,4-dioxotetrahydropyrimidin-1(2H)-yl)phenoxy)acetic acid

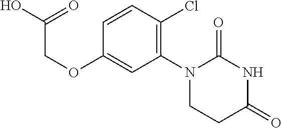 [SMILES: O=C(O)COc1ccc(Cl)c(N2CCC(=O)NC2=O)c1]

To a mixture of methyl 2-(4-chloro-3-(2,4-dioxotetrahydropyrimidin-1(2H)-yl)phenoxy)acetate (310 mg, 0.991 mmol) in THF (6 mL) was added lithium hydroxide monohydrate (74.9 mg, 1.784 mmol) at RT under argon. The RM was stirred at RT for 30 min. An aq. solution of HCl 1 M was added and the RM was concentrated to remove THF. The resulting white suspension was diluted with a cold 0.1N HCl solution and filtered off. The filter cake was washed with water and dried yielding the title compound as a white solid (222 mg). Method LCMS1: Rt=0.48 min; [M+H]⁺=299.1.

### ILB-18: 2-(3-(2,4-Dioxotetrahydropyrimidin-1(2H)-yl)phenoxy)acetaldehyde

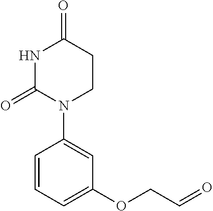 [SMILES: O=CCOc1cccc(N2CCC(=O)NC2=O)c1]

### Step 1: tert-Butyl (3-(allyloxy)phenyl)carbamate

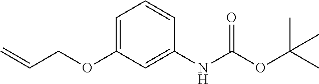 [SMILES: C=CCOc1cccc(NC(=O)OC(C)(C)C)c1]

To a solution of tert-butyl (3-hydroxyphenyl)carbamate (CAS No. [19962-06-2], commercially available, 5.025 g, 24.02 mmol) in DMF (200 mL) under argon at RT was added potassium carbonate (5.0 g, 36.2 mmol) followed by allyl bromide (CAS No. [106-95-6], commercially available, 2.4 mL, 28.4 mmol) which was added dropwise. The resulting RM was stirred for 3 days at RT and was filtered then rinsed with diethyl ether. The filtrate was taken up in Et₂O and washed with a large amount of water and then with brine. The organic phase was dried over MgSO₄ and then evaporated to dryness to afford the title compound as a pale beige solid (5.59 g). Method LCMS1: Rt=1.20 min; [M+H]⁺=250.

### Step 2: 3-(Allyloxy)aniline

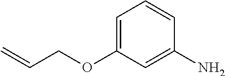

To a solution of tert-butyl (3-(allyloxy)phenyl)carbamate (5.547 g, 21.14 mmol) in DCM (80 mL) was added TFA (8.0 mL, 104 mmol). The resulting solution was stirred at RT overnight. RM was concentrated in vacuo. The crude was taken up in DCM and washed with sat. bicarbonate aq. solution. The organic layer was dried over MgSO₄ and evaporated to dryness to afford the title compound (3181 mg). Method LCMS1: Rt=0.74 min; [M+H]⁺=150.1.

### Step 3: 3,3′-((3-(Allyloxy)phenyl)azanediyl)dipropanoic acid

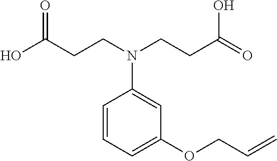 [SMILES: C=CCOc1cccc(N(CCC(=O)O)CCC(=O)O)c1]

To a suspension of 3-(allyloxy)aniline (CAS No. [74900-81-5], commercially available, 3174 mg, 18.51 mmol) in water (5 mL) was added acrylic acid (CAS No. [79-10-7], commercially available, 8 mL, 117 mmol) at RT and the RM was heated at 70° C. under argon for 1.5 h. The RM was allowed to cool to RT and adsorbed on Isolute® and purified by normal phase flash chromatography eluting with methanol in DCM (from 0 to 10%) to afford the title compound as a brown foam (5.27 g). Method LCMS1: Rt=0.79 min; [M+H]⁺=294.1.

### Step 4: 1-(3-(Allyloxy)phenyl)dihydropyrimidine-2,4(1H,3H)-dione

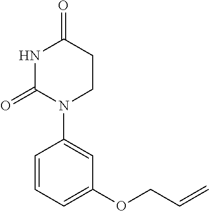 [SMILES: C=CCOc1cccc(N2CCC(=O)NC2=O)c1]

A suspension of 3,3′-((3-(allyloxy)phenyl)azanediyl)dipropanoic acid (5.27 g, 16.89 mmol) and urea (1.5 g, 24.98 mmol) in AcOH (20 mL) was heated at 120° C. under argon overnight. The RM was partially evaporated and then allowed to cool to RT. 10% aq. HCl solution was added and the RM was cooled to 0° C. and then filtered over a P4 filter frit. 3105 mg of the solid containing about 55% of the product was obtained. The filtrate also contained about 56% of the product. They were both purified separately. The solid was purified by normal phase flash chromatography eluting with methanol in DCM (from 0 to 10%) to afford the title compound as a yellow solid (2383 mg but with about 75% purity). The filtrate was purified by reverse phase chromatography on a Redisep® C18 column eluting with ACN in an aq. solution of TFA (0.1%) (from 2% to 90%) to afford, after freeze drying, 1213 mg of the title compound (purity 95%). Method LCMS1: Rt=0.74 min; [M+H]⁺=247.1.

### Step 5: 2-(3-(2,4-Dioxotetrahydropyrimidin-1(2H)-yl)phenoxy)acetaldehyde

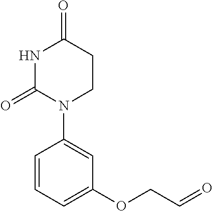 [SMILES: O=CCOc1cccc(N2CCC(=O)NC2=O)c1]

A solution of 1-(3-(allyloxy)phenyl)dihydropyrimidine-2,4(1H,3H)-dione (460 mg, 1.775 mmol) in anhydrous DCM (10.0 mL) was cooled to −78° C. Ozone generator was used and the resulting RM was stirred for 10 min. The RM was allowed to warm up to RT and triphenylphosphine polymer bound (2218 mg, 7.10 mmol) was added to destroy ozonolides. RM was stirred for 30 minutes at RT and then filtered over Celite® filter aid and washed with DCM to remove triphenylphosphine oxide. The filtrate was evaporated to dryness yielding the title compound as a white solid (435 mg). Method LCMS2: Rt=0.76 min; [M+H]+=249.1.

### ILB-19: 2-(3-(2,4-dioxotetrahydropyrimidin-1(2H)-yl)-4-methylphenoxy)acetaldehyde

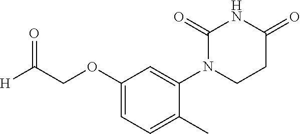 [SMILES: Cc1ccc(OCC=O)cc1N1CCC(=O)NC1=O]

### Step 1: 4-(allyloxy)-1-methyl-2-nitrobenzene

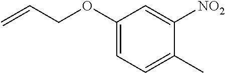

A mixture of 4-methyl-3-nitrophenol (CAS No. [2042-14-0], 15.3 g, 100 mmol) and K₂CO₃ (27.8 g, 200 mmol) in ACN (100 mL) was stirred at RT, then allyl bromide (CAS No. [106-95-6], 15.5 g, 130 mmol) was added, and the mixture was stirred at RT for 16 h. The mixture was filtered and the resulting solution was concentrated in vacuo to afford the title compound 4-(allyloxy)-1-methyl-2-nitrobenzene as a yellow oil (19 g). Method XF: Rt=1.39 min.

### Step 2: 5-(allyloxy)-2-methylaniline

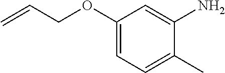

A mixture of 4-(allyloxy)-1-methyl-2-nitrobenzene (19 g, 100 mmol) and Zn (39 g, 600 mmol) in EtOH (250 mL) was stirred at RT, then AcOH (9 g, 75 mmol) was added and the mixture was stirred at RT for 16 h. After filtration, the solution was concentrated in vacuo, the residue was poured into EtOAc (500 mL), and water (200 mL) was added. The mixture was basified with K₂CO₃ to reach pH=9. The organic layer was separated, dried over Na₂SO₄, and concentrated in vacuo to afford the title compound 5-(allyloxy)-2-methylaniline as a yellow solid (17.4 g). Method XF: Rt=1.13 min; [M+H]⁺=164.

### Step 3: 3-((5-(allyloxy)-2-methylphenyl)amino)propanoic acid

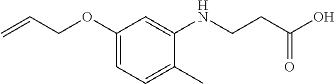 [SMILES: C=CCOc1ccc(C)c(NCCC(=O)O)c1]

A mixture of 5-(allyloxy)-2-methylaniline (17.4 g, 100 mmol) and acrylic acid (CAS No. [79-10-7], 12.4 g, 200 mmol) in toluene (50 mL) was stirred at 100° C. for 16 h. The solvent was removed in vacuo to afford the title compound 3-((5-(allyloxy)-2-methylphenyl)amino)propanoic acid as a brown oil (26 g). Method XF: Rt=0.78 min; [M+H]⁺=236.

### Step 4: 1-(5-(allyloxy)-2-methylphenyl)dihydropyrimidine-2,4(1H,3H)-dione

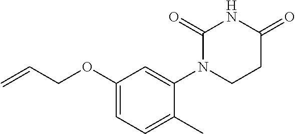 [SMILES: C=CCOc1ccc(C)c(N2CCC(=O)NC2=O)c1]

A mixture of 3-((5-(allyloxy)-2-methylphenyl)amino)propanoic acid (26 g, 100 mmol) and urea (CAS No. [57-13-6], 48 g, 800 mmol) in AcOH (500 mL) was stirred at 120° C. for 30 h. The solvent was removed in vacuo, the residue was poured into water (500 mL) and the resulting mixture was adjusted to pH=7 with NaHCO₃. Then the solid was filtered, washed with water and MTBE to afford the title compound as a light pink solid (16 g). Method XF: Rt=1.05 min; [M+H]⁺=261.

### Step 5: 2-(3-(2,4-dioxotetrahydropyrimidin-1(2H)-yl)-4-methylphenoxy)acetaldehyde

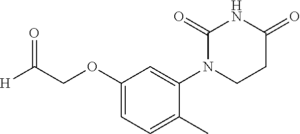 [SMILES: Cc1ccc(OCC=O)cc1N1CCC(=O)NC1=O]

A mixture of 1-(5-(allyloxy)-2-methylphenyl)dihydropyrimidine-2,4(1H,3H)-dione (8 g, 30 mmol) in DCM (200 mL) was stirred at −78° C., then bubbled with 03 for 20 min. The mixture was then bubbled with N₂ at −78° C. for 30 min, Me₂S (15 mL) was added and the mixture was stirred at −78° C. for 2 h. The solvent was removed and the residue was purified by flash chromatography on silica gel eluting with a 1:3 mixture of THF in DCM to afford the title compound as a white solid (6.1 g). Method XF: Rt=0.65 min; [M+H]⁺=263. ¹H NMR (500 MHz, DMSO-d₆) δ 10.34-10.30 (m, 1H) 9.67 (s, 1H) 7.18-7.16 (m, 1H) 6.97-6.75 (m, 2H) 4.84-4.64 (m, 2H) 3.77-3.75 (m, 1H) 3.51-3.47 (m, 1H) 2.74-2.64 (m, 2H) 2.10 (s, 3H).

### ILB-20: 2-(3-(2,4-Dioxotetrahydropyrimidin-1(2H)-yl)-4-methylphenoxy)acetic acid

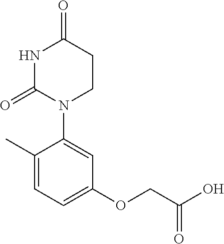 [SMILES: Cc1ccc(OCC(=O)O)cc1N1CCC(=O)NC1=O]

### Step 1: Methyl 2-(4-methyl-3-nitrophenoxy)acetate

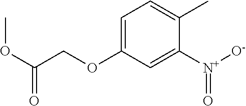 [SMILES: COC(=O)COc1ccc(C)c([N+](=O)[O-])c1]

To a solution of 4-methyl-3-nitrophenol (6.34 g, 41.4 mmol) in acetone (140 mL) was added cesium carbonate (2023 g, 62.1 mmol). Methyl bromoacetate (5.10 mL, 53.8 mmol) was added and the RM was stirred at 50° C. for 1 h. The RM was cooled to RT, and diluted with water and extracted with Et₂O three times. The organic phase was washed with brine, dried over MgSO₄ and concentrated. The crude was purified by chromatography on silica gel eluting with MeOH in DCM (from 0% to 12.5%) yielding the title compound as a beige solid (1183 mg). ¹H NMR (400 MHz, DMSO-d₆) δ 7.51 (d, J=2.8 Hz, 1H), 7.40 (d, J=8.5 Hz, 1H), 7.24 (dd, J=8.5, 2.9 Hz, 1H), 4.90 (s, 2H), 3.69 (s, 3H), 2.41 (s, 3H).

### Step 2: Methyl 2-(3-amino-4-methylphenoxy)acetate

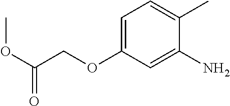

To a solution of methyl 2-(4-methyl-3-nitrophenoxy)acetate (9120 mg, 39.6 mmol) in MeOH (100 mL) at RT under argon was added palladium 10% on carbon (421 mg, 0.396 mmol). The RM was stirred at RT under hydrogen atmosphere for 18 h. The RM was filtered through Celite® filter aid and rinsed with MeOH. The filtrate was concentrated, yielding the title compound (7411 mg), directly used in next step without further purification. Method LCMS1: Rt=0.70 min; [M+H]⁺=196.2.

### Step 3: 3-((5-(2-Methoxy-2-oxoethoxy)-2-methylphenyl)amino)propanoic acid

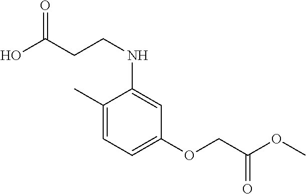 [SMILES: COC(=O)COc1ccc(C)c(NCCC(=O)O)c1]

To a mixture of methyl 2-(3-amino-4-methylphenoxy)acetate (7350 mg, 35.0 mmol) in H₂O (10 mL) was added acrylic acid (15 mL, 219 mmol) at RT. The RM was stirred at 70° C. overnight. The residue was cooled to RT, adsorbed on Isolute®, and purified by chromatography on silica gel eluting with MeOH in DCM (from 0% to 25%) yielding the title compound (23.7 g). Method LCMS1: Rt=0.76 min; [M+H]⁺=268.2.

### Step 4: 2-(3-(2,4-Dioxotetrahydropyrimidin-1(2H)-yl)-4-methylphenoxy)acetic acid

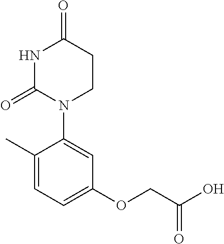 [SMILES: Cc1ccc(OCC(=O)O)cc1N1CCC(=O)NC1=O]

A mixture of 3-((5-(2-methoxy-2-oxoethoxy)-2-methylphenyl)amino)propanoic acid (23.7 g, 35.00 mmol) and urea (3.15 g, 52.5 mmol) in AcOH (60 mL) was stirred at 120° C. under argon overnight. An HCl solution 4 M in water (50 mL) was added and the RM was refluxed for 45 min. The RM was cooled to RT, then stirred at 0° C. and filtered. The filter cake was rinsed with MTBE and dried yielding the title compound as a beige solid (4.31 g). Method LCMS1: Rt=0.49 min; [M+H]⁺=279.2.

### ILB-21: 1-(4-(2,2-dimethoxyethoxy)phenyl)dihydropyrimidine-2,4(1H,3H)-dione

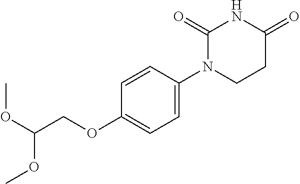 [SMILES: COC(COc1ccc(N2CCC(=O)NC2=O)cc1)OC]

2-Bromo-1,1-dimethoxyethane (4.54 mL, 38.4 mmol) was added to a mixture of ILB-36, step 1 (8 g, 38.4 mmol), potassium iodide (7 g, 42.3 mmol) and potassium carbonate (8 g, 57.6 mmol) at 115° C. in 100 mL DMF. The RM was stirred at 115° C. for 16 h. After cooling to RT, the solids were removed by filtration, and washed with ACN. The filtrate was concentrated on the rotavap, the residue dissolved in 20 mL DMSO and purified by reverse phase flash chromatography (C18, 275 g) eluting with 5-40% ACN/water over 25 min. Lyophilization yielded the title compound (4.1 g). Method XQ: Rt=0.63 min; [M+H]⁺=295.3. ¹H NMR (400 MHz, Chloroform-d) δ 7.65 (s, 1H), 7.26-7.19 (m, 2H), 7.02-6.94 (m, 2H), 4.75 (t, J=5.2 Hz, 1H), 4.03 (d, J=5.2 Hz, 2H), 3.84 (t, J=6.7 Hz, 2H), 3.48 (s, 6H), 2.84 (t, J=6.7 Hz, 2H).

### ILB-22: 3-(2,4-Dioxotetrahydropyrimidin-1(2H)-yl)-4-hydroxybenzoic acid

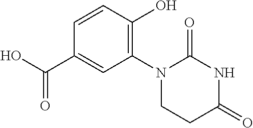 [SMILES: O=C1CCN(c2cc(C(=O)O)ccc2O)C(=O)N1]

To a white suspension of 3-(2,4-dioxotetrahydropyrimidin-1(2H)-yl)-4-methoxybenzoic acid (ILB-26, 50 mg, 0.189 mmol) in dry ACN (1.9 mL) flushed with N₂ was added aluminum iodide (CAS No. [7784-23-8], 231 mg, 0.568 mmol). The resulting yellow mixture was flushed again with N₂ then stirred at 80° C. for 2.5 h, then allowed to stand overnight at RT. The RM was diluted with ACN and water, adsorbed on Isolute®, concentrated, and purified by reverse phase chromatography on a Redisep® C18 column eluting with ACN in an aq. solution of TFA (0.1%) to afford, after freeze drying, the title compound (39 mg). Method LCMS1: Rt=0.41 min; [M−H]-=249.0. ¹H NMR (400 MHz, DMSO-d₆) δ 2.71 (t, J=6.71 Hz, 2H), 3.62 (t, J=6.71 Hz, 2H), 6.99 (d, J=8.36 Hz, 1H), 7.67-7.88 (m, 2H), 10.33 (s, 1H), 10.50 (s, 1H), 12.60 (s, 1H).

### ILB-24: 3-(2,4-dioxotetrahydropyrimidin-1(2H)-yl)-4-methylbenzoic acid

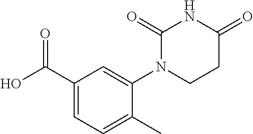 [SMILES: Cc1ccc(C(=O)O)cc1N1CCC(=O)NC1=O]

This compound was prepared as described in PCT/IB2019/052346 intermediate 8.

### ILB 25: 4-chloro-3-(2,4-dioxotetrahydropyrimidin-1(2H)-yl)benzoic acid

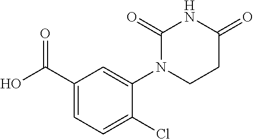 [SMILES: O=C1CCN(c2cc(C(=O)O)ccc2Cl)C(=O)N1]

This compound was prepared as described in PCT/IB2019/052346 compound 37, step 4.

### ILB-26: 3-(2,4-dioxotetrahydropyrimidin-1(2H)-yl)-4-methoxybenzoic acid

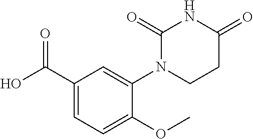 [SMILES: COc1ccc(C(=O)O)cc1N1CCC(=O)NC1=O]

This compound was prepared as described in PCT/IB2019/052346 intermediate 5.

### ILB-27: 3-(2,4-dioxotetrahydropyrimidin-1(2H)-yl)-4-fluorobenzoic acid

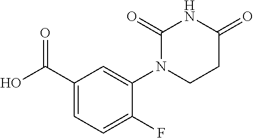 [SMILES: O=C1CCN(c2cc(C(=O)O)ccc2F)C(=O)N1]

This compound was prepared as described in PCT/IB2019/052346 intermediate 9.

### ILB-28: 3-(2,4-dioxotetrahydropyrimidin-1(2H)-yl)benzoic acid

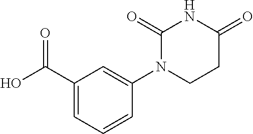 [SMILES: O=C1CCN(c2cccc(C(=O)O)c2)C(=O)N1]

This compound was prepared as described in PCT/IB2019/052346 compound 12, step 8.

### ILB-29: 5-(2,4-dioxotetrahydropyrimidin-1(2H)-yl)-2-fluoro-4-methylbenzoic acid

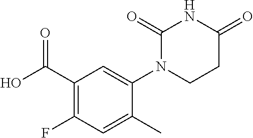 [SMILES: Cc1cc(F)c(C(=O)O)cc1N1CCC(=O)NC1=O]

This compound was prepared as described in PCT/IB2019/052346 intermediate 22.

### ILB-30: 5-(2,4-Dioxotetrahydropyrimidin-1(2H)-yl)-6-methylnicotinic acid

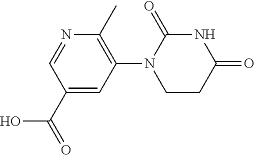 [SMILES: Cc1ncc(C(=O)O)cc1N1CCC(=O)NC1=O]

To a cloudy solution of ethyl 5-(2,4-dioxotetrahydropyrimidin-1(2H)-yl)-6-methylnicotinate (ILB-82, 22 mg, 0.079 mmol) in dry ACN (2 mL) flushed with N₂ at RT was added aluminum iodide (97 mg, 0.238 mmol). The yellow-brown mixture was stirred at 80° C. for 4.5 h, then the RM was diluted with ACN and water, adsorbed on Isolute®, concentrated until dryness and purified by reverse phase chromatography on a Redisep® C18 column eluting with ACN in an aq. solution of TFA (0.1%) (from 2% to 100%) to afford, after freeze drying, the title compound as a white solid TFA salt (22 mg). Method LCMS1: Rt=0.19 min; [M+H]⁺=250.1.

### ILB-33: 1-(3-ethynylphenyl)dihydropyrimidine-2,4(1H,3H)-dione

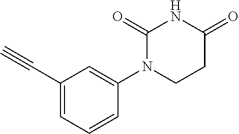 [SMILES: C#Cc1cccc(N2CCC(=O)NC2=O)c1]

### Step 1: 3-((3-((Trimethylsilyl)ethynyl)phenyl)amino)propanoic acid

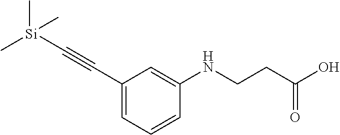 [SMILES: C[Si](C)(C)C#Cc1cccc(NCCC(=O)O)c1]

A mixture of 3-((trimethylsilyl)ethynyl)aniline (155 mg, 0.819 mmol) and acrylic acid (225 μL, 3.27 mmol) was stirred at RT for 30 minutes, then stirred at 50° C. for 3 h. The reaction mixture was dissolved in MeOH and the crude mixture purified by reverse phase preparative HPLC using Method XN to afford the title compound as a TFA salt (0.11 g). Method LCMS1: Rt=1.13 min; [M+H]⁺=262.2.

### Step 2: 1-(3-((Trimethylsilyl)ethynyl)phenyl)dihydropyrimidine-2,4(1H,3H)-dione

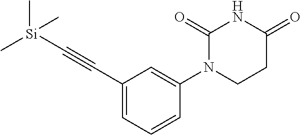 [SMILES: C[Si](C)(C)C#Cc1cccc(N2CCC(=O)NC2=O)c1]

To 3-((3-((trimethylsilyl)ethynyl)phenyl)amino)propanoic acid (0.11 g, 0.332 mmol) was added urea (0.998 g, 16.62 mmol) followed by AcOH (2.85 mL, 49.9 mmol). The reaction mixture was stirred at 120° C. overnight. The reaction mixture was concentrated to dryness and the residue diluted with EtOAc. The organic phase was washed with aqueous HCl solution and brine, dried over Na₂SO₄, filtered and concentrated to dryness. The crude mixture purified by reverse phase preparative HPLC using method XN. The appropriate fractions were combined, treated with NaHCO₃, the ACN evaporated down and the resultant aqueous extracted with EtOAc. The organic phase was separated and concentrated to dryness to afford the title compound as an off-white solid (0.05 g). Method LCMS1: Rt=1.05 min; [M+H]⁺=288.2.

### Step 3: 1-(3-Ethynylphenyl)dihydropyrimidine-2,4(1H,3H)-dione

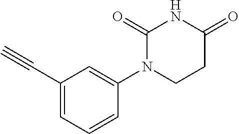 [SMILES: C#Cc1cccc(N2CCC(=O)NC2=O)c1]

A solution of 1-(3-((trimethylsilyl)ethynyl)phenyl)dihydropyrimidine-2,4(1H,3H)-dione (0.11 g, 0.384 mmol) in THF (3 mL) was cooled using an ice-bath, to which TBAF 1.0M in THF (0.461 mL, 0.461 mmol) was added. The reaction mixture was stirred while cooling with an ice bath for 90 minutes. The reaction mixture was concentrated to dryness. The crude mixture purified by reverse phase preparative HPLC using method XN-A to afford the title compound as a TFA salt (0.19 g). Method LCMS1: Rt=0.63 min; [M+H]⁺=215.1.

### ILB-34: 1-(3-(allyloxy)phenyl)dihydropyrimidine-2,4(1H,3H)-dione

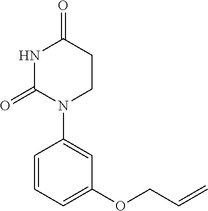 [SMILES: C=CCOc1cccc(N2CCC(=O)NC2=O)c1]

### Step 1: tert-butyl (3-(allyloxy)phenyl)carbamate

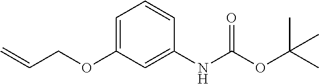 [SMILES: C=CCOc1cccc(NC(=O)OC(C)(C)C)c1]

To a 500 mL round bottom flask were added tert-butyl (3-hydroxyphenyl)carbamate (5025 mg, 24.02 mmol), potassium carbonate (5000 mg, 36.20 mmol) and DMF (200 mL). Allyl bromide (2.4 mL, 28.40 mmol) was added dropwise and the RM was stirred at RT for 3 days. The RM was filtered and rinsed with Et₂O. The filtrate was taken up in Et₂O and washed with water and brine. The organic phase was dried over MgSO₄ and evaporated to dryness, yielding the title compound as a beige solid (5599 mg). Method LCMS1: Rt=1.20 min; [M+H]⁺=250.2.

### Step 2: 3-(allyloxy)aniline

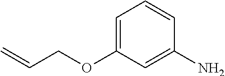

To a 250 mL round bottom flask were added tert-butyl (3-(allyloxy)phenyl)carbamate (5547 mg, 21.14 mmol) and DCM (80 mL). TFA (8 mL, 104 mmol) was added and the RM was stirred at RT overnight. The RM was concentrated. The residue was taken up in DCM and washed with sat. aq. NaHCO₃ solution. The organic was dried over MgSO₄ and evaporated to dryness, yielding the title compound as an orange liquid (3181 mg). Method LCMS1: Rt=0.74 min; [M+H]⁺=150.1.

### Step 3: 3,3′-((3-(allyloxy)phenyl)azanediyl)dipropionic acid

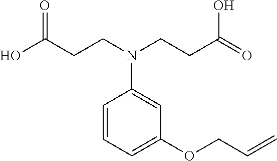 [SMILES: C=CCOc1cccc(N(CCC(=O)O)CCC(=O)O)c1]

To a 100 mL round bottom flask were added 3-(allyloxy)aniline (3174 mg, 18.51 mmol) and water (5 mL). Acrylic acid (8 mL, 117.00 mmol) was added at RT and the mixture was stirred at 70° C. for 1.5 h. The RM was cooled to RT, adsorbed on Isolute®, and purified by chromatography on silica gel eluting with iPrOH (from 0% to 10%) in DCM, yielding the title compound as a brown foam (5.27 g). Method LCMS1: Rt=0.79 min; [M+H]⁺=294.1.

### Step 4: 1-(3-(allyloxy)phenyl)dihydropyrimidine-2,4(1H,3H)-dione

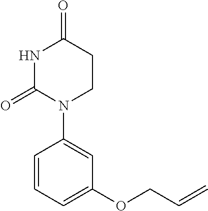 [SMILES: C=CCOc1cccc(N2CCC(=O)NC2=O)c1]

A suspension of 3,3′-((3-(allyloxy)phenyl)azanediyl)dipropionic acid (713 mg, 0.778 mmol) and urea (200 mg, 3.33 mmol) in AcOH (6 mL) was stirred at 120° C. overnight. The RM was partially evaporated. The residue was purified by reversed phase chromatography on a RediSep® Gold HP C18 column (50 g) eluting with ACN (from 2% to 100%) in an aq. solution of TFA (0.1%). Fractions containing pure target compound were filtered through PL-HCO₃ MP SPE cartridges and freeze dried to afford the title compound (176 mg). Method LCMS1: Rt=0.74 min; [M+H]⁺=247.1.

### ILB-35: 1-(4-(2-(piperazin-1-yl)ethoxy)phenyl)dihydropyrimidine-2,4(1H,3H)-dione

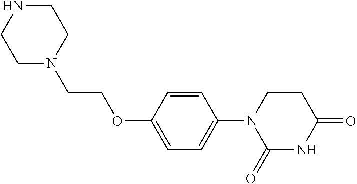 [SMILES: O=C1CCN(c2ccc(OCCN3CCNCC3)cc2)C(=O)N1]

### Step 1: tert-Butyl 4-(2-(4-(2,4-dioxotetrahydropyrimidin-1(2H)-yl)phenoxy)ethyl)piperazine-1-carboxylate

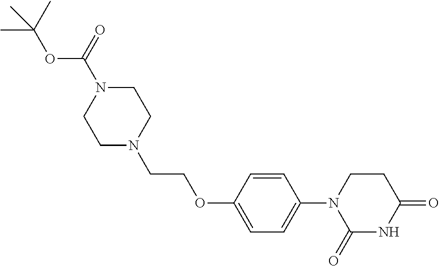 [SMILES: CC(C)(C)OC(=O)N1CCN(CCOc2ccc(N3CCC(=O)NC3=O)cc2)CC1]

To a stirred brown suspension of 1-(4-hydroxyphenyl)dihydropyrimidine-2,4(1H,3H)-dione (ILB-36 step 1, 600 mg, 2.91 mmol), 1-Boc-4-(2-hydroxyethyl)piperazine (804 mg, 3.49 mmol), and PPh₃ (916 mg, 3.49 mmol) in 29 mL of dry THF flushed with N₂ and cooled down at 0-5° C. with an ice-water bath was added slowly DEAD 40% in toluene (1.382 mL, 3.49 mmol.) over 10 minutes. The resulting RM was then stirred in the bath for 1 h, before being allowed to stir at RT over 3 days. RM was diluted with ACN and concentrated until dryness. Crude: 3.09 g. The dark residue was then re-dissolved in a minimum of ACN, adsorbed on Isolute and purified by reverse phase chromatography on a Redisep® C18 column of 275 g eluting with ACN/aq. Solution of TFA 0.1% to afford the title compound as the TFA salt (550 mg). Method LCMS1: Rt=0.58 min; [M+H]⁺=419.3.

### Step 2: 1-(4-(2-(piperazin-1-yl)ethoxy)phenyl)dihydropyrimidine-2,4(1H,3H)-dione

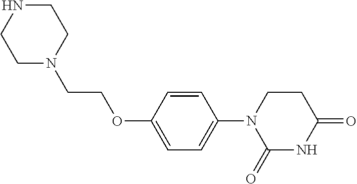 [SMILES: O=C1CCN(c2ccc(OCCN3CCNCC3)cc2)C(=O)N1]

To a colorless solution of tert-Butyl 4-(2-(4-(2,4-dioxotetrahydropyrimidin-1(2H)-yl)phenoxy)ethyl)piperazine-1-carboxylate (548 mg, 1.029 mmol) in 15 mL of DCM was added TFA (2.38 mL, 30 eq). The resulting RM (solution) was stirred at RT for 1 h. RM was diluted with DCM and concentrated until dryness, then co-evaporated with DCM (2×) and dried under HV pump a few hours. The residue was then lyophilized over a long weekend to afford the TFA salt of the title compound as a beige hygroscopic solid (590 mg). Method LCMS1: Rt=0.36 min; [M+H]⁺=319.2.

### ILB-36: 1-(4-(piperidin-4-yloxy)phenyl)dihydropyrimidine-2,4(1H,3H)-dione

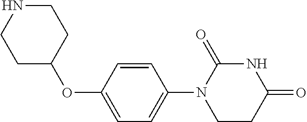 [SMILES: O=C1CCN(c2ccc(OC3CCNCC3)cc2)C(=O)N1]

### Step 1: 1-(4-hydroxyphenyl)dihydropyrimidine-2,4(1H,3H)-dione

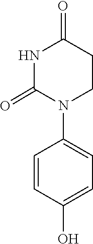 [SMILES: O=C1CCN(c2ccc(O)cc2)C(=O)N1]

A purple mixture of 4-aminophenol (CAS No. [123-30-8], 5.5 g, 50.4 mmol), methyl acrylate (CAS No. [96-33-3], 5 mL, 55.5 mmol), AcOH (0.5 mL, 8.73 mmol) and hydroquinone (CAS No. [123-31-9], 20 mg, 0.182 mmol) in iPrOH (5 mL) was refluxed at 85° C. for 17 h, then concentrated. The resulting crude mixture was dissolved in 20% aq. HCl (20 mL), stirred for 1 h at RT and refluxed overnight, then the RM was concentrated in vacuo. To the resulting crude mixture was added urea (CAS No. [57-13-6], 6.1 g, 102 mmol), then it was heated overnight at 120° C. in AcOH (20 mL), and cooled down to RT. Then, 10% aq. HCl was added, the solution was cooled to 0° C. and then filtered through a frit. The solid was dried under reduced pressure to afford the title compound as dark crystals (4.325 g). Method LCMS2: Rt=0.72 min; [M+H]⁺=207.1.

### Step 2: tert-Butyl 4-(4-(2,4-dioxotetrahydropyrimidin-1(2H)-yl)phenoxy)piperidine-1-carboxylate

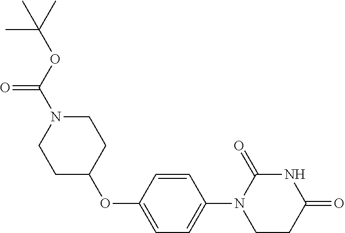 [SMILES: CC(C)(C)OC(=O)N1CCC(Oc2ccc(N3CCC(=O)NC3=O)cc2)CC1]

To a stirred brown mixture of 1-(4-hydroxyphenyl)dihydropyrimidine-2,4(1H,3H)-dione (300 mg, 1.455 mmol), 1-Boc-4-hydroxypiperidine (CAS No. [109384-19-2], 351 mg, 1.746 mmol), and PPh₃ (CAS No. [603-35-0], 458 mg, 1.746 mmol) in dry THF (14.5 mL) flushed with N₂ and cooled down to 0-5° C. was slowly added DEAD 40% in toluene (CAS No. [1972-28-7], 691 μL, 1.746 mmol) over 10 min. The resulting RM was then stirred at 0-5° C. for 10 min before being allowed to stir at RT. After 3 h at RT, the RM was diluted with EtOAc (75 mL) and water (30 mL). More EtOAc (30 mL) and brine (15 mL) were added to help separation. Layers were separated, the aq. layer was extracted with EtOAc (1×30 mL). The combined organic layers were dried over MgSO₄, filtered and concentrated under HV to afford a light brown solid (1.47 g). Purification of the crude product by flash chromatography on silica gel eluting with 1-20% (DCM/iPrOH 80/20) in DCM afforded the title compound as an off-white solid (388 mg). Method LCMS1: Rt=0.96 min; [M+H]⁺=390.3.

### Step 3: 1-(4-(piperidin-4-yloxy)phenyl)dihydropyrimidine-2,4(1H,3H)-dione

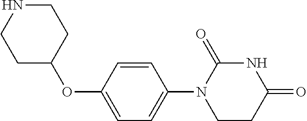 [SMILES: O=C1CCN(c2ccc(OC3CCNCC3)cc2)C(=O)N1]

To a colorless solution of tert-butyl 4-(4-(2,4-dioxotetrahydropyrimidin-1(2H)-yl)phenoxy)piperidine-1-carboxylate (387 mg, 0.994 mmol) in DCM (3.8 mL) was added TFA (2.297 mL, 29.8 mmol). The resulting solution was stirred at RT for 1 h, diluted with DCM and concentrated until dryness, then co-evaporated with DCM (1×), and dried under HV pump to afford a resin. The resin was redissolved in a mixture of ACN and water, then freeze dried to afford the title compound as an off-white solid TFA salt (419 mg). Method LCMS1: Rt=0.37 min; [M+H]⁺=290.3.

### ILB-37: 1-(3-((6-Aminohexyl)oxy)phenyl)dihydropyrimidine-2,4(1H,3H)-dione

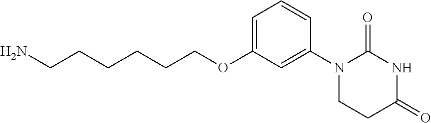 [SMILES: NCCCCCCOc1cccc(N2CCC(=O)NC2=O)c1]

### Step 1: 3,3′-((3-hydroxyphenyl)azanediyl)dipropanoic acid

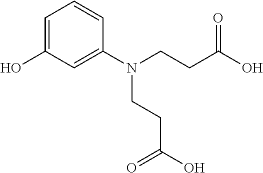 [SMILES: O=C(O)CCN(CCC(=O)O)c1cccc(O)c1]

To a suspension of 3-aminophenol (CAS No. [591-27-5], commercially available, 10.9 g, 100 mmol) in water (9 mL) at RT was added acrylic acid (CAS No. [79-10-7], commercially available, 18.5 mL, 295 mmol) and the RM was heated at 70° C. under argon for 3 h. The RM was allowed to cool to RT, EtOH (18 mL) was added, and the RM was stored at 4° C. for 12 h. The heterogeneous mixture was filtered over a P4 filter frit, the solid was carefully washed with EtOAc and then dried in vacuum over P205 affording the title compound as a white powder (15.77 g). Method LCMS1: Rt=0.44 min; [M+H]⁺=254.1.

### Step 2: 1-(3-hydroxyphenyl)dihydropyrimidine-2,4(1H,3H)-dione

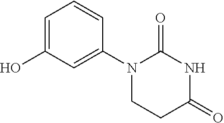 [SMILES: O=C1CCN(c2cccc(O)c2)C(=O)N1]

A suspension of 3,3′-((3-hydroxyphenyl)azanediyl)dipropanoic acid (1266 mg, 5 mmol) and urea (450 mg, 7.5 mmol) in AcOH (7.5 mL) was heated at 130° C. under argon overnight. The RM was allowed to cool to RT, 10% aq. solution of HCl (20 mL) was added and the RM was heated until reflux for 30 min. The RM was allowed to cool to RT and 3/4 of the solvent was evaporated yielding a heterogeneous orange mixture. The mixture was cooled to 0° C. for 20 min and filtered over a P4 filter frit. The solids were washed with an ice cold aq. solution of HCl (0.1 M) (2×3 mL) and then dried under vacuum over P205 affording the title compound as a slightly yellow powder (506 mg). Method LCMS1: Rt=0.44 min; [M+H]⁺=207.1.

### Step 3: tert-butyl (6-(3-(2,4-dioxotetrahydropyrimidin-1(2H)-yl)phenoxy)hexyl)carbamate

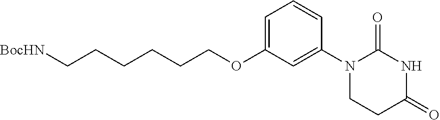 [SMILES: CC(C)(C)OC(=O)NCCCCCCOc1cccc(N2CCC(=O)NC2=O)c1]

To a solution of tert-butyl (6-bromohexyl)carbamate (CAS No. [142356-33-0], commercially available, 326 mg, 1.164 mmol) in DMF (3 mL) under argon at RT was added 1-(3 hydroxyphenyl)dihydropyrimidine-2,4(1H,3H)-dione (200 mg, 0.970 mmol) followed by potassium carbonate (402 mg, 2.91 mmol). The resulting RM was stirred overnight at RT. The RM was diluted with water (30 mL) and EtOAc (30 mL) and after stirring for 5 min the phases were separated. The aq. phase was extracted with EtOAc (5×20 mL) and the combined organic phases were dried over MgSO₄. Evaporation gave a colorless oil which was adsorbed on Isolute® and purified by reverse phase chromatography on a Redisep® Rf Gold 50 g HP C18 column eluting with ACN in an aq. solution of TFA (0.1%) (from 2 to 100%) to afford 170 mg of a colorless oil containing the expected product. It was then purified further by SFC using method XU on a Reprospher PEI column (250×30 mm, 100 A, 5 μm) eluting with methanol from 16% to 22% to afford the title compound (110 mg). Method LCMS1: Rt=1.05 min; [M+H]⁺=406.3.

### Step 4: 1-(3-((6-aminohexyl)oxy)phenyl)dihydropyrimidine-2,4(1H,3H)-dione

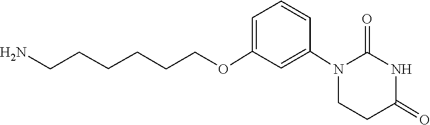 [SMILES: NCCCCCCOc1cccc(N2CCC(=O)NC2=O)c1]

To tert-butyl (6-(3-(2,4-dioxotetrahydropyrimidin-1(2H)-yl)phenoxy)hexyl)carbamate (105 mg, 0.259 mmol) was added HCl (4.0 M) in dioxane (4.0 mL, 16.00 mmol) at RT. The resulting solution was stirred at RT for 1 h, then evaporated to dryness and further dried under vacuum over P205 overnight to afford the title compound as an HCl salt (85 mg). Method LCMS1: Rt=0.51 min; [M+H]⁺=306.2.

### ILB-38: 1-(2,6-difluoro-4-(piperidin-4-ylethynyl)phenyl)dihydropyrimidine-2,4(1H,3H)-dione

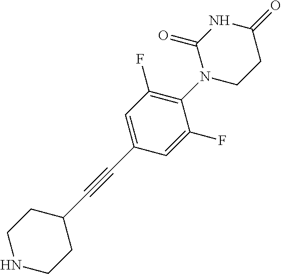 [SMILES: O=C1CCN(c2c(F)cc(C#CC3CCNCC3)cc2F)C(=O)N1]

The mixture of 1-(4-bromo-2,6-difluorophenyl)dihydropyrimidine-2,4(1H,3H)-dione (ILB-45 step 2, 20 mg, 0.066 mmol), 1-(prop-2-yn-1-yl)piperazine (20 mg, 0.066 mmol), 1-(prop-2-yn-1-yl)piperazine (48.8 mg, 0.393 mmol) copper (I) iodide (4.99 mg, 0.026 mmol), tetrakis(triphenylphosphine)palladium (0) (15.15 mg, 0.013 mmol), TEA (0.091 mL, 0.656 mmol) in DMF (0.5 mL) was stirred at 115° C. for 0.5 h. After cooling to RT, the mixture was diluted EtOAc, washed with water, brine, the organic phase was dried over anhydrous sodium sulfate, filtered and concentrated. The residue was purified with reverse phase C18 chromatography, eluted with 10-100% 0.1% TFA in ACN/water to provide the desired product as a white solid TFA salt (4 mg). Method XR-A: Rt=0.65 min; MS [M+H]+=334.2. ¹H NMR (400 MHz, DMSO-d6) δ 10.67 (s, 1H), 8.45 (s, 1H), 7.65-7.06 (m, 2H), 3.70 (t, J=6.7 Hz, 2H), 3.36-2.78 (m, 7H), 2.15-1.92 (m, 4H).

### ILB-39: 1-(4-(5-iodopent-1-yn-1-yl)phenyl)dihydropyrimidine-2,4(1H,3H)-dione

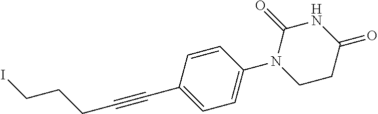 [SMILES: O=C1CCN(c2ccc(C#CCCCI)cc2)C(=O)N1]

### Step 1: 1-(4-(5-hydroxypent-1-yn-1-yl)phenyl)dihydropyrimidine-2,4(1H,3H)-dione

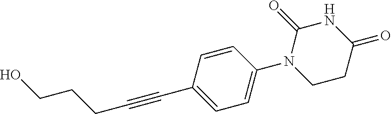 [SMILES: O=C1CCN(c2ccc(C#CCCCO)cc2)C(=O)N1]

A yellow solution of 1-(4-iodophenyl)dihydropyrimidine-2,4(1H,3H)-dione (CAS No. [1528991-21-0], 203 mg, 0.642 mmol), DBU (0.232 mL, 1.541 mmol) and Pd(PPh₃)₄ (18.55 mg, 0.016 mmol) in DMSO (2 mL) was bubbled with Argon for 5 min, then pent-4-yn-1-ol (0.034 mL, 0.357 mmol) was added, and the RM was stirred at 80° C. for 1 h. Then, TPGS-750-M 2 wt. % solution in water (=DL-α-Tocopherol methoxypolyethylene glycol succinate solution) (CAS No. [1309573-60-1], 1 mL) was added, the mixture was stirred at 80° C. for 1 h and allowed to stand overnight at RT. The RM was dissolved in a mixture of EtOAc and MeOH (9:1, 50 mL) and washed with sat. aq. Na₂CO₃ (2×50 mL). The aqueous layers were extracted with a mixture of EtOAc and MeOH (9:1, 50 mL). The organic layers were combined and evaporated to afford a yellow solid, which was purified by flash chromatography on silica gel eluting with 5-100% EtOAc in CHX to afford the title compound as a white solid (118 mg). Method LCMS1: Rt=0.59 min; [M+H]⁺=273.2.

### Step 2: 1-(4-(5-iodopent-1-yn-1-yl)phenyl)dihydropyrimidine-2,4(1H,3H)-dione

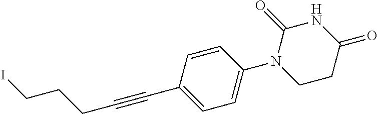 [SMILES: O=C1CCN(c2ccc(C#CCCCI)cc2)C(=O)N1]

To a yellow suspension of 1-(4-(5-hydroxypent-1-yn-1-yl)phenyl)dihydropyrimidine-2,4(1H,3H)-dione (162 mg, 0.595 mmol) and imidazole (CAS No. [288-32-4], 60.8 mg, 0.892 mmol) in THF (2 mL) was added DCM (2 mL), followed by PPh₃ (203 mg, 0.773 mmol) and 12 (226 mg, 0.892 mmol) in one portion. The vial was covered with aluminum foil and the brown suspension was stirred at RT for 4 h. The RM was dissolved in EtOAc (50 mL) and extracted with sat. aq. Na₂CO₃ (50 mL), followed by 20% aq. Na₂S₂O₃ (50 mL) and water (50 mL). The aqueous layers were extracted with EtOAc (30 mL). The organic layers were combined and evaporated to afford a yellow solid, which was purified by flash chromatography on silica gel eluting with 5-100% EtOAc in CHX to afford the title compound as a white solid (186 mg). Method LCMS1: Rt=0.99 min; [M+H]⁺=383.1.

### ILB-40: 1-(4-(3-(1-oxa-4,9-diazaspiro[5.5]undecan-4-yl)prop-1-yn-1-yl)phenyl)dihydropyrimidine-2,4(1H,3H)-dione

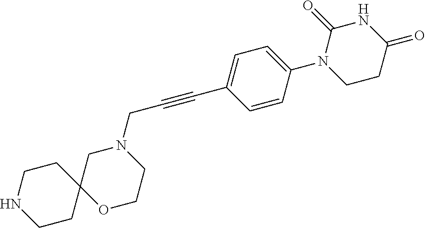 [SMILES: O=C1CCN(c2ccc(C#CCN3CCOC4(CCNCC4)C3)cc2)C(=O)N1]

### Step 1: tert-butyl 4-(3-(4-(2,4-dioxotetrahydropyrimidin-1(2H)-yl)phenyl)prop-2-yn-1-yl)-1-oxa-4,9-diazaspiro[5.5]undecane-9-carboxylate

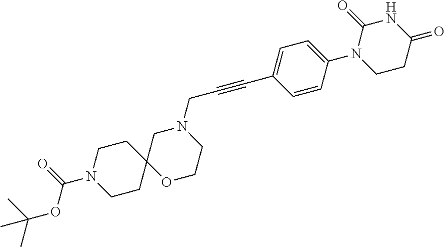 [SMILES: CC(C)(C)OC(=O)N1CCC2(CC1)CN(CC#Cc1ccc(N3CCC(=O)NC3=O)cc1)CCO2]

To a solution of tert-butyl 4-(prop-2-yn-1-yl)-1-oxa-4,9-diazaspiro[5.5]undecane-9-carboxylate (Intermediate DD, 51.2 mg, 0.174 mmol), 1-(4-iodophenyl)dihydropyrimidine-2,4(1H,3H)-dione (Intermediate BB, 50 mg, 0.158 mmol), PdCl₂(PPh₃)₂ (5.55 mg, 7.91 μmol), and CuI (1.506 mg, 7.91 μmol) in DMF (1582 μL) under an atmosphere of nitrogen, was added TEA (110 μL, 0.791 mmol). The reaction mixture was stirred at 80° C. for 90 min, then was cooled to room temperature and diluted with EtOAc and water. The layers were separated and the aqueous layer was extracted with EtOAc (3×20 mL). The combined organic extracts were washed with brine (2×20 mL), dried over magnesium sulfate, filtered, and concentrated. The crude residue was purified via flash chromatography (0-30% MeOH/DCM) to afford the title compound (50 mg). Method XV: Rt=0.95 min; [M+H]⁺=483.4.

### Step 2: 1-(4-(3-(1-oxa-4,9-diazaspiro[5.5]undecan-4-yl)prop-1-yn-1-yl)phenyl)dihydropyrimidine-2,4(1H,3H)-dione

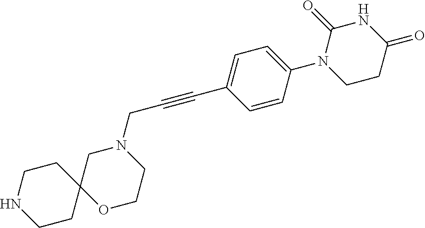 [SMILES: O=C1CCN(c2ccc(C#CCN3CCOC4(CCNCC4)C3)cc2)C(=O)N1]

A solution of 4 N HCl in dioxane (777 μL, 3.11 mmol) was added to tert-butyl 4-(3-(4-(2,4-dioxotetrahydropyrimidin-1(2H)-yl)phenyl)prop-2-yn-1-yl)-1-oxa-4,9-diazaspiro[5.5]undecane-9-carboxylate (50 mg, 0.104 mmol), and the resulting solution was stirred at RT for 18 h. The solvent was concentrated to afford the title compound as an HCl salt (43.4 mg). Method XV: Rt=0.71 min; [M+H]⁺=383.3.

### ILB-41: 1-(4-(4-(4-(piperazine-1-carbonyl)piperazin-1-yl)but-1-yn-1-yl)phenyl)dihydropyrimidine-2,4(1H,3H)-dione

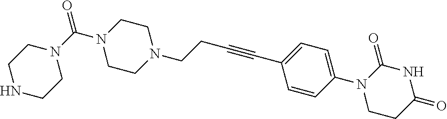 [SMILES: O=C1CCN(c2ccc(C#CCCN3CCN(C(=O)N4CCNCC4)CC3)cc2)C(=O)N1]

### Step 1: tert-butyl 4-(4-(but-3-yn-1-yl)piperazine-1-carbonyl)piperazine-1-carboxylate

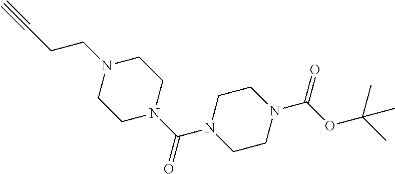 [SMILES: C#CCCN1CCN(C(=O)N2CCN(C(=O)OC(C)(C)C)CC2)CC1]

To a solution of tert-butyl 4-(piperazine-1-carbonyl)piperazine-1-carboxylate (150 mg, 0.503 mmol) in ACN (5.0 mL), was added K₂CO₃ (208 mg, 1.508 mmol), followed by 4-bromobut-1-yne (0.090 mL, 1.005 mmol). The resulting solution was stirred at 75° C. for 72 h. The RM was then cooled to RT, diluted with EtOAc, and washed with water (2×25 mL). The combined aqueous layers were then extracted again with EtOAc (3×15 mL), and the combined organic layers were dried over sodium sulfate, filtered, and concentrated to afford the title compound as a white solid (160 mg). Method XV: Rt=0.87 min; [M+H]⁺=351.3.

### Step 2: 1-(4-(4-(4-(piperazine-1-carbonyl)piperazin-1-yl)but-1-yn-1-yl)phenyl)dihydropyrimidine-2,4(1H,3H)-dione

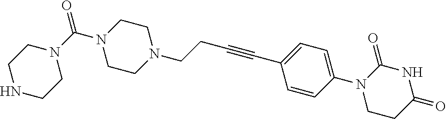 [SMILES: O=C1CCN(c2ccc(C#CCCN3CCN(C(=O)N4CCNCC4)CC3)cc2)C(=O)N1]

To a solution of tert-butyl 4-(4-(but-3-yn-1-yl)piperazine-1-carbonyl)piperazine-1-carboxylate (14.41 mg, 0.041 mmol), 1-(4-iodophenyl)dihydropyrimidine-2,4(1H,3H)-dione (CAS No. [1528991-21-0], Intermediate BB, 10 mg, 0.032 mmol), PdCl₂(PPh₃)₂ (1.110 mg, 1.582 μmol), and CuI (0.301 mg, 1.582 μmol) in dioxane (300 μL) was added TEA (150 μL, 1.076 mmol), and the resulting solution was stirred at 80° C. for 30 min. The RM was then cooled to RT and a solution of HCl (4M in dioxane) (395 μL, 1.582 mmol) was added and the RT was stirred at RT for 72 h. The solution was filtered, diluted with a 1:1:1 solution of H₂O:ACN:DMSO and purified via preparative HPLC (XBridge C18 30×50 mm 10-30% MeCN/H₂O (5 mM NH₄OH) to afford the title compound as a white powder (5 mg). Method XV: Rt=0.61 min; [M+H]⁺=439.4.

### ILB-43: tert-Butyl (3-(4-(2,4-dioxotetrahydropyrimidin-1(2H)-yl)-3,5-difluorophenyl)prop-2-yn-1-yl)carbamate

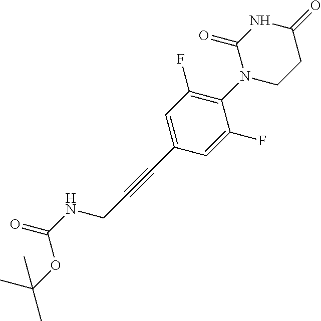 [SMILES: CC(C)(C)OC(=O)NCC#Cc1cc(F)c(N2CCC(=O)NC2=O)c(F)c1]

The mixture of 1-(4-bromo-2,6-difluorophenyl)dihydropyrimidine-2,4(1H,3H)-dione (ILB-45 step 2, 20 mg, 0.066 mmol), tert-butyl prop-2-yn-1-ylcarbamate (61.0 mg, 0.393 mmol), 1-(prop-2-yn-1-yl)piperazine (48.8 mg, 0.393 mmol) copper (I) iodide (4.99 mg, 0.026 mmol), tetrakis(triphenylphosphine)palladium (0) (15.15 mg, 0.013 mmol), TEA (0.091 mL, 0.656 mmol) in DMF (0.5 mL) was stirred at 115° C. for 0.5 hr. After cooling to RT, the mixture was diluted EtOAc, washed with water, brine, dried over anhydrous sodium sulfate, filtered and concentrated. The residue was purified with flash chromatography (0-10% MeOH/DCM) to provide the desired product as a light brown solid (10 mg). Method XR-A: Rt=1.81 min; [M-55]⁺=324.1. ¹H NMR (400 MHz, Acetonitrile-d₃) δ 8.40 (s, 1H), 7.24-7.08 (m, 2H), 5.68 (s, 1H), 4.07 (d, J=6.0 Hz, 2H), 3.74 (t, J=6.6 Hz, 2H), 2.79 (s, 2H), 1.46 (s, 9H).

### ILB-44: 1-(4-(4-aminobut-1-yn-1-yl)phenyl)dihydropyrimidine-2,4(1H,3H)-dione

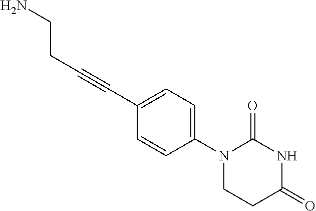 [SMILES: NCCC#Cc1ccc(N2CCC(=O)NC2=O)cc1]

### Step 1: tert-Butyl (4-(4-(2,4-dioxotetrahydropyrimidin-1(2H)-yl)phenyl)but-3-yn-1-yl)carbamate

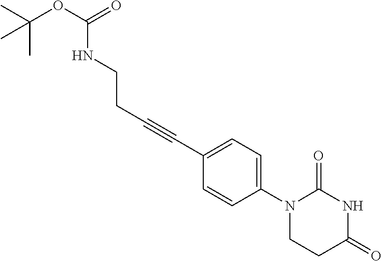 [SMILES: CC(C)(C)OC(=O)NCCC#Cc1ccc(N2CCC(=O)NC2=O)cc1]

To 1-(4-iodophenyl)dihydropyrimidine-2,4(1H,3H)-dione (CAS No.: [1528991-21-0], 300 mg, 0.949 mmol), CuI (36.2 mg, 0.190 mmol) and PdCl₂(PPh₃)₂ (66.6 mg, 0.095 mmol) in DMF (8 mL) was slowly added TEA (0.794 mL, 5.69 mmol). The RM was stirred under argon atmosphere at 80° C. for 30 min. The residue was purified by reverse phase chromatography on a Redisep® C18 column eluting with ACN in an aq. solution of TFA (0.1%) to afford, after freeze drying, the title compound as a grey solid (346 mg). Method LCMS1: Rt=0.87 min; [M−H]⁻=356.2.

### Step 2: 1-(4-(4-aminobut-1-yn-1-yl)phenyl)dihydropyrimidine-2,4(1H,3H)-dione

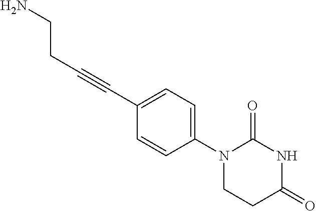 [SMILES: NCCC#Cc1ccc(N2CCC(=O)NC2=O)cc1]

To a solution of tert-butyl (4-(4-(2,4-dioxotetrahydropyrimidin-1(2H)-yl)phenyl)but-3-yn-1-yl)carbamate (346 mg, 0.661 mmol) in dioxane (2 mL) was added HCl 4 N in dioxane (6.61 mL, 26.4 mmol) and the RM was stirred at RT for 1.5 h. The mixture was concentrated, then dried in vacuo to afford the title compound as an orange powder HCl salt (250 mg). Method LCMS2: Rt=0.66 min; [M+H]⁺=258.2. ¹H NMR (400 MHz, DMSO-d₆) δ 10.42 (s, 1H), 8.06 (s, 3H), 7.49-7.43 (m, 2H), 7.36-7.31 (m, 2H), 3.80 (t, J=6.6 Hz, 2H), 3.03 (q, J=6.7 Hz, 2H), 2.78 (t, J=7.1 Hz, 2H), 2.70 (t, J=6.6 Hz, 2H).

### ILB-45: 1-(2,6-difluoro-4-(3-(piperazin-1-yl)prop-1-yn-1-yl)phenyl)dihydropyrimidine-2,4(1H,3H)-dione

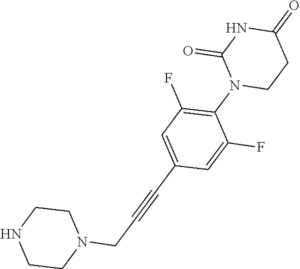 [SMILES: O=C1CCN(c2c(F)cc(C#CCN3CCNCC3)cc2F)C(=O)N1]

### Step 1: Methyl 3-((4-bromo-2,6-difluorophenyl)amino)propanoate

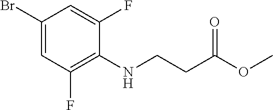 [SMILES: COC(=O)CCNc1c(F)cc(Br)cc1F]

Trifluoromethanesulfonic acid (0.021 mL, 0.240 mmol) was added to the mixture of 4-bromo-2,6-difluoroaniline (1 g, 4.81 mmol), methyl acrylate (4.33 mL, 48.1 mmol). The mixture was stirred at 80° C. overnight. After cooling to RT, the mixture was diluted with EtOAc, washed with water, brine, the organic phase was dried over anhydrous sodium sulfate, filtered and concentrated. The residue was purified with flash chromatography (0-30% EtOAc/heptane) to provide the desired product as a glassy solid (470 mg). Method XQ: Rt=1.07 min; MS [M+H]⁺=294.1.

### Step 2: 1-(4-bromo-2,6-difluorophenyl)dihydropyrimidine-2,4(1H,3H)-dione

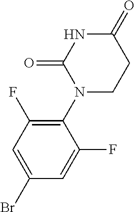 [SMILES: O=C1CCN(c2c(F)cc(Br)cc2F)C(=O)N1]

2,2,2-Trichloroacetyl isocyanate (169 mg, 0.898 mmol) in THF (2 mL) was added to the mixture of methyl 3-((4-bromo-2,6-difluorophenyl)amino)propanoate (240 mg, 0.816 mmol) in THF (8 mL) at 0° C. After stirred for 30 minutes, ammonia in methanol (2.332 mL, 16.32 mmol) was added to the mixture. The resulting mixture was stirred at RT overnight. Then, more ammonia in methanol (2.332 mL, 16.32 mmol) was added, and the mixture was allowed to warm up to 60° C. After stirred overnight, the mixture was concentrated. The residue was purified with flash chromatography (0-60% EtOAc/heptane) to provide the desired product as a solid (130 mg). Method XQ: Rt=0.71 min; [M+H]⁺=304.9.

### Step 3: 1-(2,6-difluoro-4-(3-(piperazin-1-yl)prop-1-yn-1-yl)phenyl)dihydropyrimidine-2,4(1H,3H)-dione

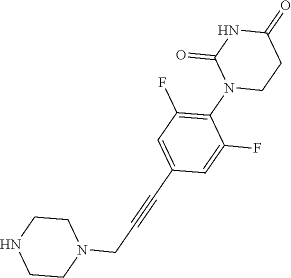 [SMILES: O=C1CCN(c2c(F)cc(C#CCN3CCNCC3)cc2F)C(=O)N1]

The mixture of 1-(4-bromo-2,6-difluorophenyl)dihydropyrimidine-2,4(1H,3H)-dione (20 mg, 0.066 mmol), 1-(prop-2-yn-1-yl)piperazine (48.8 mg, 0.393 mmol), copper (I) iodide (4.99 mg, 0.026 mmol), tetrakis(triphenylphosphine)palladium (0) (15.15 mg, 0.013 mmol), TEA (0.091 mL, 0.656 mmol) in DMF (0.5 mL) was stirred at 115° C. for 0.5 h. After cooling to RT, the mixture was diluted EtOAc, washed with water, brine, the organic phase was dried over anhydrous sodium sulfate, filtered and concentrated. The residue was purified with reverse phase C18 chromatography, eluted with 10-100% 0.1% TFA in ACN/water to provide the desired product as a white solid TFA salt (2.7 mg). Method XR-A: Rt=0.62 min; [M+H]⁺=349.1. ¹H NMR (400 MHz, DMSO-d₆) δ 10.69 (s, 1H), 8.75 (s, 1H), 7.33-6.94 (m, 2H), 3.79-3.42 (m, 6H), 3.28-3.05 (m, 4H), 2.87-2.65 (m, 5H).

### ILB-46: 1-(4-(piperidin-4-yl)phenyl)dihydropyrimidine-2,4(1H,3H)-dione

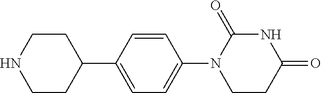 [SMILES: O=C1CCN(c2ccc(C3CCNCC3)cc2)C(=O)N1]

### Step 1: tert-Butyl 4-(4-(2,4-dioxotetrahydropyrimidin-1(2H)-yl)phenyl)-5,6-dihydropyridine-1(2H)-carboxylate

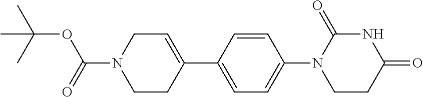 [SMILES: CC(C)(C)OC(=O)N1CC=C(c2ccc(N3CCC(=O)NC3=O)cc2)CC1]

To a mixture of 1-(4-iodophenyl)dihydropyrimidine-2,4(1H,3H)-dione (CAS No. [1528991-21-0], 200 mg, 0.633 mmol), 3,6-dihydro-2H-pyridine-1-tert-butoxycarbonyl-4-boronic acid pinacol ester (CAS No. [286961-14-6], 215 mg, 0.696 mmol) and K₃PO₄ (403 mg, 1.898 mmol) in dioxane (10.5 mL) at RT flushed with N₂ was added PdCl₂(dppf) (46.3 mg, 0.063 mmol), followed by water (2.1 mL). The RM was flushed with N₂, the vial was capped and heated under microwave irradiation at 120° C. for 30 min. The RM was diluted with ACN, adsorbed on Isolute®, concentrated until dryness and purified by flash chromatography on silica gel eluting with 1-25% (DCM/iPrOH 80/20) in DCM to afford the title compound as a yellow solid (154 mg). Method LCMS1: Rt=0.98 min; [M-tBu+H]⁺=316.3.

### Step 2: tert-Butyl 4-(4-(2,4-dioxotetrahydropyrimidin-1(2H)-yl)phenyl)piperidine-1-carboxylate

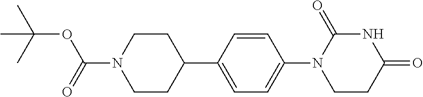 [SMILES: CC(C)(C)OC(=O)N1CCC(c2ccc(N3CCC(=O)NC3=O)cc2)CC1]

A pale yellow mixture of tert-butyl 4-(4-(2,4-dioxotetrahydropyrimidin-1(2H)-yl)phenyl)-5,6-dihydropyridine-1(2H)-carboxylate (151 mg, 0.386 mmol) in dry EtOH (12 mL) was treated with 10% palladium on activated charcoal (CAS No. [7440-05-3], 41.1 mg, 0.039 mmol) and the RM was stirred under H₂ atmosphere at RT for 20 h. Then, the RM diluted with EtOAc, filtered through Hyflo® and rinsed with a mixture of EtOH and EtOAc. The pale yellow filtrate was then concentrated until dryness to afford the title compound as a beige solid (154 mg). Method LCMS1: Rt=0.99 min; [M+H₂O]⁺=391.4.

### Step 3: 1-(4-(piperidin-4-yl)phenyl)dihydropyrimidine-2,4(1H,3H)-dione

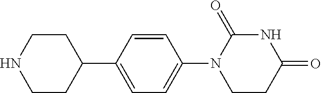 [SMILES: O=C1CCN(c2ccc(C3CCNCC3)cc2)C(=O)N1]

To a colorless solution of tert-butyl 4-(4-(2,4-dioxotetrahydropyrimidin-1(2H)-yl)phenyl)piperidine-1-carboxylate (153 mg, 0.373 mmol) in DCM (5.4 mL) was added TFA (862 μL, 11.18 mmol) and the resulting solution was stirred at RT for 1 h. The RM was diluted with DCM, concentrated until dryness, co-evaporated with DCM, then dried under HV pump and freeze dried to afford the title compound as a solid TFA salt (166 mg). Method LCMS1: Rt=0.36 min; [M+H]⁺=274.3.

### ILB-47: 1-(4-(3-(1-oxa-4,9-diazaspiro[5.5]undecan-4-yl)propyl)phenyl)dihydropyrimidine-2,4(1H,3H)-dione

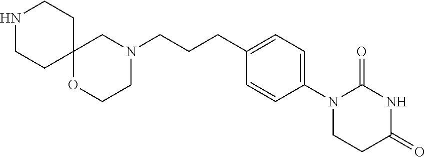 [SMILES: O=C1CCN(c2ccc(CCCN3CCOC4(CCNCC4)C3)cc2)C(=O)N1]

### Step 1: tert-Butyl 4-(3-(4-(2,4-dioxotetrahydropyrimidin-1(2H)-yl)phenyl)propyl)-1-oxa-4,9-diazaspiro[5.5]undecane-9-carboxylate

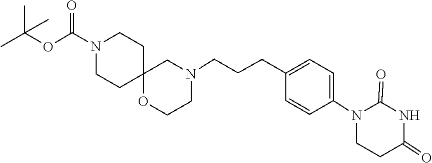 [SMILES: CC(C)(C)OC(=O)N1CCC2(CC1)CN(CCCc1ccc(N3CCC(=O)NC3=O)cc1)CCO2]

To a mixture of tert-butyl 4-(3-(4-(2,4-dioxotetrahydropyrimidin-1(2H)-yl)phenyl)prop-2-yn-1-yl)-1-oxa-4,9-diazaspiro[5.5]undecane-9-carboxylate (ILB-40 step 2, 200 mg, 0.41 mmol) in EtOH (10 mL) was added Pd/C (50 mg, 10% w/w). The mixture was purged with H₂ and heated at 25° C. for 8 h. The mixture was filtered and the filtrate was concentrated in vacuo. The residue was purified by chromatography on a 12 g silica Biotage® column eluting with EtOAc in DCM (from 30 to 90%), 20 mL/min, to afford the title compound as a white solid (122 mg). Method G: Rt=1.92 min; [M+H]⁺=487.3

### Step 2: 1-(4-(3-(1-oxa-4,9-diazaspiro[5.5]undecan-4-yl)propyl)phenyl)dihydropyrimidine-2,4(1H,3H)-dione

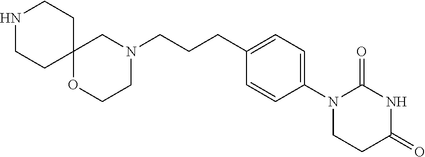 [SMILES: O=C1CCN(c2ccc(CCCN3CCOC4(CCNCC4)C3)cc2)C(=O)N1]

To a mixture of tert-butyl 4-(3-(4-(2,4-dioxotetrahydropyrimidin-1(2H)-yl)phenyl)propyl)-1-oxa-4,9-diazaspiro[5.5]undecane-9-carboxylate (118 mg, 0.24 mmol) in DCM (3 mL) was added TFA (1 mL). The mixture was stirred at 25° C. for 3 h. The mixture was concentrated in vacuo. The residue was diluted with DCM (5 mL) and basified with ammonia in MeOH (7N) to pH 9. Silica gel (100-200 mesh, 10 mL) was added and the mixture was concentrated in vacuo. The residue was purified by chromatography on a 12 g silica Biotage® column eluting with a methanolic ammonia solution (0.7 N) in DCM (from 5 to 25%), 20 mL/min, to afford the title compound as a colorless oil (85 mg). Method G: Rt=1.47 min; [M+H]⁺=387.2

### ILB-49: 1-(4-(3-(4-(4-bromo-1H-pyrazol-1-yl)piperidin-1-yl)prop-1-yn-1-yl)phenyl)dihydropyrimidine-2,4(1H,3H)-dione

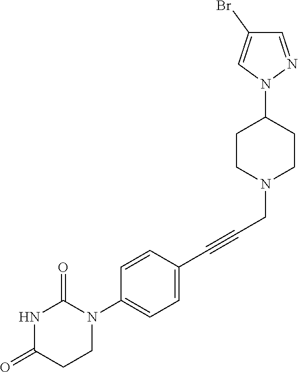 [SMILES: O=C1CCN(c2ccc(C#CCN3CCC(n4cc(Br)cn4)CC3)cc2)C(=O)N1]

### Step 1: tert-Butyl 4-(4-bromo-1H-pyrazol-1-yl)piperidine-1-carboxylate

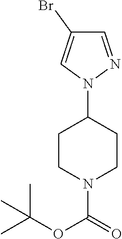 [SMILES: CC(C)(C)OC(=O)N1CCC(n2cc(Br)cn2)CC1]

4-bromo-1H-pyrazole (CAS No. [2075-45-8], 1.104 g, 7.51 mmol) was dissolved in DMF (15.02 mL) and cooled down to 0° C. NaH (0.330 g, 8.26 mmol) was added, the RM was stirred at 0° C. for 1 h, then tert-butyl 4-((methylsulfonyl)oxy)piperidine-1-carboxylate (CAS No. [141699-59-4], 2.098 g, 7.51 mmol) was added and the resulting suspension was stirred at 100° C. for 1 h. The RM was quenched with water and extracted with EtOAc. The organic layer was washed with water and brine, dried over Na₂SO₄, filtered, and concentrated. The residue was purified by flash chromatography on silica gel eluting with 0-50% EtOAc in CHX to afford the title compound (1.876 g). Method LCMS1: Rt=1.16 min; [M+H]⁺=330.2/332.2.

### Step 2: 4-(4-bromo-1H-pyrazol-1-yl)piperidine

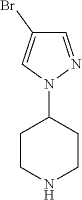 [SMILES: Brc1cnn(C2CCNCC2)c1]

tert-Butyl 4-(4-bromo-1H-pyrazol-1-yl)piperidine-1-carboxylate (1.850 g, 5.60 mmol) was dissolved in DCM (56 mL), then TFA (2.158 mL, 28 mmol) was added and the RM was stirred at RT for 18 h. More TFA (2.158 mL, 28.0 mmol) was added and the RM was stirred at RT for 3 h. The RM was basified with NH₄OH and extracted with DCM. The organic layer was washed with brine, dried over Na₂SO₄, filtered, and concentrated to afford the title compound (1.250 g). Method LCMS1: Rt=0.44 min; [M+H]⁺=230.1/232.1.

### Step 3: 4-(4-bromo-1H-pyrazol-1-yl)-1-(prop-2-yn-1-yl)piperidine

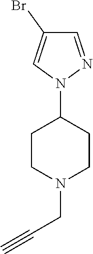

4-(4-bromo-1H-pyrazol-1-yl)piperidine (1.24 g, 5.39 mmol) was dissolved in THF (53.9 mL), Cs₂CO₃ (1.756 g, 5.39 mmol) was added, followed by propargyl bromide 80% in toluene (0.581 mL, 5.39 mmol) and the RM was stirred at RT for 18 h. The RM was quenched with water and extracted with EtOAc, the organic layer was washed with brine, dried over Na₂SO₄, filtered, concentrated and purified by flash chromatography on silica gel eluting with 0-100% EtOAc in CHX to afford the title compound (1.140 g). Method LCMS1: Rt=0.51 min; [M+H]⁺=268.2 /270.2.

### Step 4: 1-(4-(3-(4-(4-bromo-1H-pyrazol-1-yl)piperidin-1-yl)prop-1-yn-1-yl)phenyl)dihydropyrimidine-2,4(1H,3H)-dione

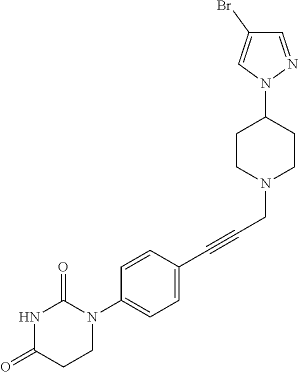 [SMILES: O=C1CCN(c2ccc(C#CCN3CCC(n4cc(Br)cn4)CC3)cc2)C(=O)N1]

1-(4-iodophenyl)dihydropyrimidine-2,4(1H,3H)-dione (CAS No. [1528991-21-0], 50 mg, 0.158 mmol) was dissolved in DMF (1.582 mL), 4-(4-bromo-1H-pyrazol-1-yl)-1-(prop-2-yn-1-yl)piperidine (42.4 mg, 0.158 mmol) was added, followed by CuI (6.03 mg, 0.032 mmol), TEA (132 μL, 0.949 mmol), and PdCl₂(PPh₃)₂ (11.10 mg, 0.016 mmol). The RM was stirred at RT for 18 h, then filtered through a silica gel pre-column, washing with DCM/MeOH (95/5), and concentrated in vacuo to afford a residue. The residue was purified by flash chromatography on silica gel eluting with 0-5% MeOH in DCM to afford the title compound (52 mg). Method LCMS1: Rt=0.61 min; [M+H]⁺=456.2/458.2.

### ILB-50: tert-Butyl (6-(3-(2,4-dioxotetrahydropyrimidin-1(2H)-yl)phenoxy)hexyl)(methyl)carbamate

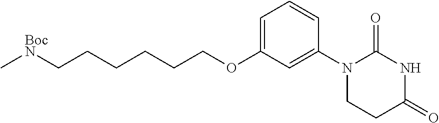 [SMILES: CN(CCCCCCOc1cccc(N2CCC(=O)NC2=O)c1)C(=O)OC(C)(C)C]

### Step 1: 3,3′-((3-Hydroxyphenyl)azanediyl)dipropanoic acid

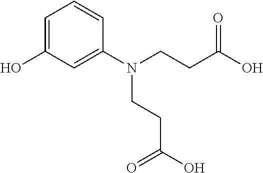 [SMILES: O=C(O)CCN(CCC(=O)O)c1cccc(O)c1]

To a suspension of 3-aminophenol (CAS No. [591-27-5], commercially available, 10.9 g, 100 mmol) in water (9 mL) at RT was added acrylic acid (CAS No. [79-10-7], commercially available, 18.5 mL, 295 mmol) and the RM was heated at 70° C. under argon for 3 h. The RM was allowed to cool to RT, EtOH (18 mL) was added and the RM was stored at 4° C. for 12 h. The heterogeneous mixture was filtered over a P4 filter frit, the solid was carefully washed with EtOAc and then dried in vacuum over P205 affording the title compound as a white powder (15.77 g). Method LCMS1: Rt=0.44 min; [M+H]⁺=254.1.

### Step 2: 1-(3-Hydroxyphenyl)dihydropyrimidine-2,4(1H,3H)-dione

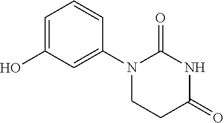 [SMILES: O=C1CCN(c2cccc(O)c2)C(=O)N1]

A suspension of 3,3′-((3-hydroxyphenyl)azanediyl)dipropanoic acid (1266 mg, 5 mmol) and urea (450 mg, 7.5 mmol) in AcOH (7.5 mL) was heated at 130° C. under argon overnight. The RM was allowed to cool to RT, 10% aq. solution of HCl (20 mL) was added and the RM was heated until reflux for 30 min. The RM was allowed to cool to RT and 3/4 of the solvent was evaporated yielding a heterogeneous orange mixture. The mixture was cooled to 0° C. for 20 min and filtered over a P4 filter frit. The solids were washed with an ice cold aq. solution of HCl (0.1 M) (2×3 mL) and then dried under vacuum over P205 affording the title compound as a slightly yellow powder (506 mg). Method LCMS1: Rt=0.44 min; [M+H]⁺=207.1.

### Step 3: tert-Butyl (6-((tert-butyldimethylsilyl)oxy)hexyl)carbamate

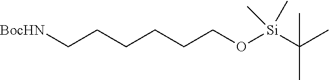 [SMILES: CC(C)(C)OC(=O)NCCCCCCO[Si](C)(C)C(C)(C)C]

To a solution of tert-butyl (6-hydroxyhexyl)carbamate (CAS No. [75937-12-1], commercially available, 5.0 g, 23.01 mmol) and imidazole (2.036 g, 29.9 mmol) in DCM (30 mL) under argon at 0° C. was added dropwise a solution of tert-butylchlorodimethylsilane (3.81 g, 25.3 mmol) in DCM (10 mL) over 15 min. After the addition, the RM was stirred at 0° C. for 15 min, then the cooling bath was removed and stirring was continued at RT for 2 days. The RM was filtered over a P4 filter frit and the solid was washed with PE (3×20 mL). The combined filtrates were evaporated, redissolved in DCM (150 mL) and washed with an aq. solution of HCl (1 M) (3×30 mL) and brine (2×30 mL), dried over Na₂SO₄, and concentrated under vacuum to afford a colorless oil. It was then adsorbed on Isolute® and purified by normal phase flash chromatography on a Redisep® Rf 220 g column eluting with EtOAc in CHX (from 0 to 10%) to afford the title compound as a colorless oil (7.35 g).

### Step 4: tert-Butyl (6-((tert-butyldimethylsilyl)oxy)hexyl)(methyl)carbamate

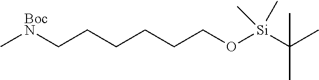 [SMILES: CN(CCCCCCO[Si](C)(C)C(C)(C)C)C(=O)OC(C)(C)C]

To a suspension of NaH (60% in mineral oil) (3.64 g, 91 mmol) in THF (80 mL) at 0° C. under argon was added dropwise a solution of tert-butyl (6-((tert-butyldimethylsilyl)oxy)hexyl)carbamate (7.3 g, 22.02 mmol) in THF (20 mL) over 15 min. After the addition the RM was stirred at 0° C. for 30 min, then iodomethane (3.38 mL, 54 mmol) was added dropwise over 10 min and after the addition the reaction mixture was allowed to RT. The resulting RM was stirred overnight at RT. Aq. sat. NH₄Cl (100 mL) was carefully added to the reaction mixture at 0° C., the mixture was allowed to warm to RT under stirring for 30 min and filtered over a P4 filter frit. The solids were washed with MTBE (3×25 mL) and the phases were separated. The aq. phase was extracted with MTBE (3×50 mL), the combined organic phases were washed with brine (2×50 mL), dried over Na₂SO₄, filtered, and concentrated under vacuum yielding a yellow oil (8.70 g). It was then adsorbed on Isolute® and purified by normal phase flash chromatography on a Redisep® Rf 220 g column eluting with EtOAc in CHX (from 0 to 10%) to afford the title compound as a colorless oil (7.03 g). ¹H NMR (400 MHz, Chloroform-d6) S 3.59 (t, J=6.5 Hz, 2H), 3.18 (t, J=7.3 Hz, 2H), 2.82 (s, 3H), 1.56-1.47 (m, 4H), 1.45 (s, 9H), 1.39-1.22 (m, 4H), 0.89 (s, 9H), 0.04 (s, 6H).

### Step 5: tert-Butyl (6-hydroxyhexyl)(methyl)carbamate

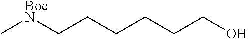 [SMILES: CN(CCCCCCO)C(=O)OC(C)(C)C]

To a solution of tert-butyl (6-((tert-butyldimethylsilyl)oxy)hexyl)(methyl)carbamate (2.0 g, 5.8 mmol) in THF (30.0 mL) was added tetrabutylammonium fluoride (1.0 M) in THF (17.36 mL, 17.36 mmol) dropwise at RT. The resulting RM was stirred overnight at RT. The solvent was removed then ice cold water (30 mL) was added and the mixture was stirred 30 min at RT. Cyclohexane (50 mL) was added and after stirring additional 10 min, the phases were separated. The aq. phase was extracted with cyclohexane (4×50 mL) and the combined organic phases were dried over Na₂SO₄, filtered, and concentrated yielding a yellow oil: 1.97 g. It was then adsorbed on Isolute® and purified by normal phase flash chromatography on a Redisep® Rf 80 g column eluting with EtOAc in cyclohexane (from 0 to 100%) to afford the title compound as a colorless oil (1.26 g). ¹H NMR (400 MHz, Chloroform-d) δ 3.63 (t, J=6.5 Hz, 2H), 3.19 (t, J=7.2 Hz, 2H), 2.84-2.79 (m, 3H), 1.62-1.46 (m, 4H), 1.45 (s, 9H), 1.42-1.34 (m, 2H), 1.34-1.24 (m, 2H).

### Step 6: tert-Butyl (6-(3-(2,4-dioxotetrahydropyrimidin-1(2H)-yl)phenoxy)hexyl)(methyl)carbamate

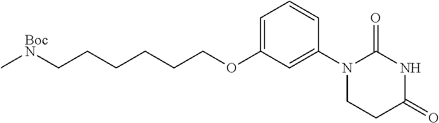 [SMILES: CN(CCCCCCOc1cccc(N2CCC(=O)NC2=O)c1)C(=O)OC(C)(C)C]

1-(3 hydroxyphenyl)dihydropyrimidine-2,4(1H,3H)-dione (step 2, 200 mg, 0.970 mmol), tert-butyl (6-hydroxyhexyl)(methyl)carbamate (step 5, 269 mg, 1.164 mmol) and triphenylphosphine (356 mg, 1.358 mmol) were added to the reaction flask which was then flushed with argon and THF (5 mL) was added via a syringe. The suspension was cooled to 0° C. and DEAD 40% in toluene (0.537 mL, 1.358 mmol) was added dropwise. After the addition, stirring was continued at 0° C. and the reaction mixture was allowed to warm to RT without withdrawing the cooling bath. The resulting RM was stirred overnight at RT. Solvent was partially evaporated and then adsorbed on Isolute® and purified by reverse phase chromatography on a Redisep® Rf Gold 50 g HP C18 column eluting with ACN in an aq. solution of TFA (0.1%) (from 2% to 100% ACN) to afford 176 mg of a colorless oil containing the expected product. It was then purified further by SFC on a Reprospher PEI column (250×30 mm, 100 Å, 5 μm) eluting with methanol from 12% to 18% to afford the title compound (105 mg). Method LCMS1: Rt=1.12 min; [M+H]⁺=420.3.

### ILB-52: 1-(4-(3-(3,9-diazaspiro[5.5]undecan-3-yl)prop-1-yn-1-yl)phenyl)dihydropyrimidine-2,4(1H,3H)-dione

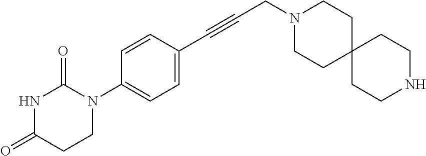 [SMILES: O=C1CCN(c2ccc(C#CCN3CCC4(CCNCC4)CC3)cc2)C(=O)N1]

### Step 1: tert-Butyl 9-(prop-2-yn-1-yl)-3,9-diazaspiro[5.5]undecane-3-carboxylate

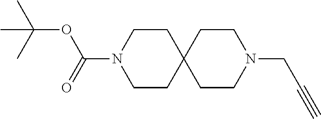 [SMILES: C#CCN1CCC2(CC1)CCN(C(=O)OC(C)(C)C)CC2]

To a stirred colorless solution of tert-butyl 3,9-diazaspiro[5.5]undecane-3-carboxylate (CAS No. [173405-78-2], 8 g, 31.5 mmol) in ACN (175 mL) at RT flushed with N₂ was added K₂CO₃ (6.52 g, 47.2 mmol) to give a white suspension. After 10 min stirring at RT, propargyl bromide 80% in toluene (4.20 mL, 37.7 mmol) was slowly added and the RM was stirred at 60° C. for 22 h. The RM was filtered to remove inorganics, rinsed with ACN, and the filtrate was concentrated until dryness to afford a yellow-orange solid. The solid was purified by flash chromatography on silica gel eluting with 10-50% EtOAc in CHX to afford the title compound as a pale yellow solid (6.31 g). Method LCMS1: Rt=0.64 min; [M+H]⁺=293.3.

### Step 2: tert-Butyl 9-(3-(4-(2,4-dioxotetrahydropyrimidin-1(2H)-yl)phenyl)prop-2-yn-1-yl)-3,9-diazaspiro[5.5]undecane-3-carboxylate

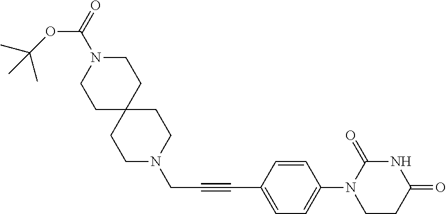 [SMILES: CC(C)(C)OC(=O)N1CCC2(CCN(CC#Cc3ccc(N4CCC(=O)NC4=O)cc3)CC2)CC1]

A mixture of 1-(4-iodophenyl)dihydropyrimidine-2,4(1H,3H)-dione (Intermediate BB, CAS No. [1528991-21-0], 300 mg, 0.949 mmol), tert-butyl 9-(prop-2-yn-1-yl)-3,9-diazaspiro[5.5]undecane-3-carboxylate (305 mg, 1.044 mmol), CuI (36.2 mg, 0.190 mmol) and PdCl₂(PPh₃)₂ (68 mg, 0.095 mmol) in dry DMF (4.5 mL) was flushed with N₂. Then, while stirring, TEA (0.789 mL, 5.69 mmol) was slowly added, the resulting yellow mixture was then flushed with N₂, heated at 80° C. for 3 h, then allowed to stand overnight at RT. The RM was diluted with DCM (30 mL) and washed with a mixture of water and brine (15 mL). The aq. layer was extracted twice with DCM (20 mL, then 10 mL). Combined organics were dried then over MgSO₄, filtered, concentrated and dried to afford a brown solid. The solid was purified by flash chromatography on silica gel eluting with 2-30% (DCM/MeOH 80/20) in DCM to afford the title compound as a brown solid (358 mg). Method LCMS1: Rt=0.69 min; [M+H]⁺=481.6.

### Step 3: 1-(4-(3-(3,9-diazaspiro[5.5]undecan-3-yl)prop-1-yn-1-yl)phenyl)dihydropyrimidine-2,4(1H,3H)-dione

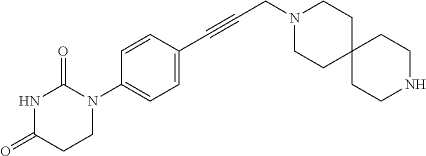 [SMILES: O=C1CCN(c2ccc(C#CCN3CCC4(CCNCC4)CC3)cc2)C(=O)N1]

tert-Butyl 9-(3-(4-(2,4-dioxotetrahydropyrimidin-1(2H)-yl)phenyl)prop-2-yn-1-yl)-3,9-diazaspiro[5.5]undecane-3-carboxylate (87 mg, 0.163 mmol) was dissolved in DCM (2 mL). TFA (2 mL, 26.0 mmol) was added and the clear solution was stirred at RT for 20 min, then evaporated on Isolute® and purified by reverse phase chromatography on a Redisep® C18 column eluting with ACN in an aq. solution of TFA (0.1%) to afford, after freeze drying, the title compound as a yellow solid bis-TFA salt (76 mg). Method LCMS1: Rt=0.32 min; [M+H]⁺=381.2. ¹H NMR (400 MHz, DMSO-d₆) δ 10.47 (s, 1H), 10.06 (s, 1H), 8.40 (s, 2H), 7.55 (d, J=8.5 Hz, 2H), 7.41 (d, J=8.5 Hz, 2H), 4.36 (s, 2H), 3.82 (t, J=6.6 Hz, 2H), 3.45 (s, 2H), 3.14 (s, 2H), 3.06 (s, 4H), 2.72 (t, J=6.6 Hz, 2H), 1.92 (s, 2H), 1.74 (s, 2H), 1.52 (s, 4H).

### ILB-53: N-(5-aminopentyl)-3-(2,4-dioxotetrahydropyrimidin-1(2H)-yl)-4-methylbenzenesulfonamide

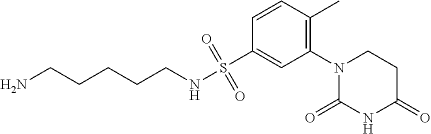 [SMILES: Cc1ccc(S(=O)(=O)NCCCCCN)cc1N1CCC(=O)NC1=O]

### Step 1: tert-Butyl (5-(4-methyl-3-nitrophenylsulfonamido)pentyl)carbamate

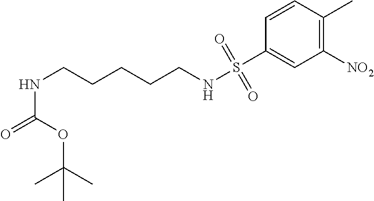 [SMILES: Cc1ccc(S(=O)(=O)NCCCCCNC(=O)OC(C)(C)C)cc1[N+](=O)[O-]]

A solution of N-Boc-cadaverine (650 mg, 3.12 mmol) and TEA (0.900 mL, 6.39 mmol) in DCM (25 mL) was cooled at 0° C., to which a solution of 4-methyl-3-nitrobenzenesulfonyl chloride (795 mg, 3.27 mmol) in DCM (50 mL) was added dropwise. The reaction was allowed to warm to RT and stirred at RT for 2 h. The RM was partially concentrated under reduced pressure and washed with water (×3), dried over magnesium sulfate, filtered and concentrated to dryness. The crude material was purified by silica gel chromatography eluting with EtOAc in CHX (from 0% to 100%) to afford the title compound (1165 mg). Method LCMS1: Rt=1.09 min; [M+H]⁺=402.3.

### Step 2: tert-Butyl (5-(3-amino-4-methylphenylsulfonamido)pentyl)carbamate

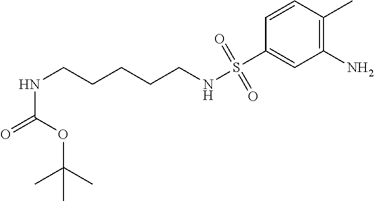 [SMILES: Cc1ccc(S(=O)(=O)NCCCCCNC(=O)OC(C)(C)C)cc1N]

A solution of tert-butyl (5-(4-methyl-3-nitrophenylsulfonamido)pentyl)carbamate (1150 mg, 2.86 mmol) in MeOH (15 mL) was purged three times with argon. Then palladium 10% on carbon (305 mg, 0.286 mmol) was added. The RM was vigorously stirred for 2 h under hydrogen atmosphere.

The RM was flushed with argon and the black suspension was filtered over a Celite® filter aid plug, rinsing with MeOH. The filtrate was concentrated to dryness to afford the title compound (1056 mg). Method LCMS1: Rt=0.96 min; [M+H]⁺=372.3.

### Step 3: 3-((5-(N-(5-((tert-Butoxycarbonyl)amino)pentyl)sulfamoyl)-2-methylphenyl)amino)propanoic acid

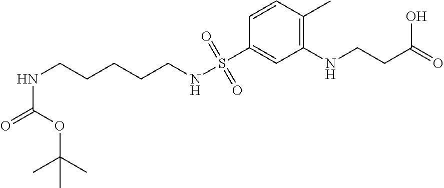 [SMILES: Cc1ccc(S(=O)(=O)NCCCCCNC(=O)OC(C)(C)C)cc1NCCC(=O)O]

To a solution of tert-butyl (5-(3-amino-4-methylphenylsulfonamido)pentyl)carbamate (1045 mg, 2.67 mmol) in water (5 mL) at RT was added acrylic acid (1.2 mL, 17.48 mmol). The resulting dark solution was stirred at 70° C. for 5 h and then at RT for 2 days. The RM was concentrated to dryness and used in the next step without further purification.

### Step 4: N-(5-Aminopentyl)-3-(2,4-dioxotetrahydropyrimidin-1(2H)-yl)-4-methylbenzenesulfonamide

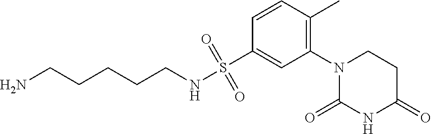 [SMILES: Cc1ccc(S(=O)(=O)NCCCCCN)cc1N1CCC(=O)NC1=O]

To a solution of 3-((5-(N-(5-((tert-butoxycarbonyl)amino)pentyl)sulfamoyl)-2-methylphenyl)amino)propanoic acid (crude, 2.56 mmol) in AcOH (10 mL) was added urea (0.5 g, 8.33 mmol). The RM was stirred at 120° C. overnight. The RM was concentrated to dryness. The crude material was purified by reverse phase chromatography on a Redisep® C18 column eluting with ACN in an aqueous solution of TFA (0.1%) (from 2% to 100%) to afford the TFA salt of the title compound as a liquid (245 mg). Method LCMS1: Rt=0.42 min; [M+H]⁺=369.2.

### ILB-54: 1-(3-((6-(Methylamino)hexyl)oxy)phenyl)dihydropyrimidine-2,4(1H,3H)-dione

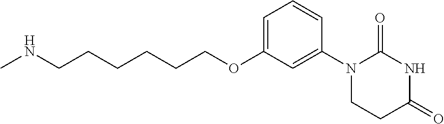 [SMILES: CNCCCCCCOc1cccc(N2CCC(=O)NC2=O)c1]

To tert-butyl (6-(3-(2,4-dioxotetrahydropyrimidin-1(2H)-yl)phenoxy)hexyl)(methyl)carbamate (ILB-50, 105 mg, 0.250 mmol) in solution in THF (5 mL) was added HCl (4 M) in dioxane (2.0 mL, 8.00 mmol). The resulting solution was stirred at RT for 2 h, then evaporated to dryness and further dried under vacuum over P205 overnight to afford the title compound as an HCl salt (85 mg). Method LCMS1: Rt=0.56 min; [M+H]⁺=320.2.

### ILB-55: 1-(4-(2-Oxo-2-(4-(piperidin-4-yloxy)piperidin-1-yl)ethoxy)phenyl)dihydropyrimidine-2,4(1H,3H)-dione

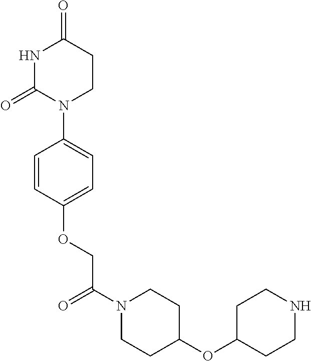 [SMILES: O=C1CCN(c2ccc(OCC(=O)N3CCC(OC4CCNCC4)CC3)cc2)C(=O)N1]

### Step 1: methyl 2-(4-((tert-butoxycarbonyl)amino)phenoxy)acetate

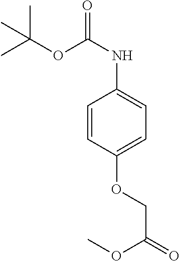 [SMILES: COC(=O)COc1ccc(NC(=O)OC(C)(C)C)cc1]

To a 250 mL round bottom flask were added tert-butyl (4-hydroxyphenyl)carbamate (7 g, 31.8 mmol), Cs₂CO₃ (11.4 g, 35.0 mmol), KI (5 mg, 0.301 mmol), and acetone (75 mL). Methyl bromoacetate was added and the RM was stirred at 70° C. for 4 h. The RM was cooled to RT, filtered and the filtrate was concentrated. The residue was diluted with EtOAc, washed with a sat. aq. solution of NaHCO₃, dried over MgSO₄, and evaporated. The residue was purified by chromatography on silica gel eluting with EtOAc (from 0% to 25%) in CHX, yielding the title compound as a solid (8.83 g). Method LCMS1: Rt=0.97 min; [M+H]⁺=282.

### Step 2: methyl 2-(4-aminophenoxy)acetate

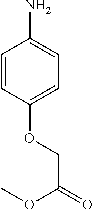

To a 100 mL round bottom flask were added methyl 2-(4-((tert-butoxycarbonyl)amino)phenoxy)acetate (8.83 g, 31.4 mmol), TFA (30 mL, 389 mmol) and 1,4-dioxane (30 mL). The RM was stirred at RT for 18 h and concentrated. The residue was diluted with DCM, the organic phase was washed with a sat. aq. solution of NaHCO₃ and dried over MgSO₄, yielding the title compound as an oil (5.35 g), which was directly used for next step without further purification. Method LCMS1: Rt=0.37 min; [M+H]⁺=182.

### Step 3: 3,3′-((4-(2-methoxy-2-oxoethoxy)phenyl)azanediyl)dipropionic acid

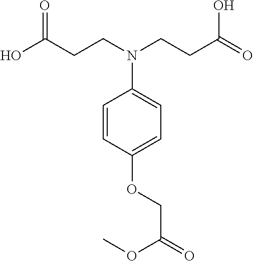 [SMILES: COC(=O)COc1ccc(N(CCC(=O)O)CCC(=O)O)cc1]

To a 100 mL round bottom flask were added methyl 2-(4-aminophenoxy)acetate (5.35 g, 25.7 mmol), acrylic acid (11 mL, 160 mmol) and water (5 mL). The RM was stirred at 70° C. for 1.5 h. The RM was cooled to RT, adsorbed on Isolute® and purified by chromatography on silica gel eluting with a mixture (4:1) of DCM and iPrOH (from 0% to 50%) in DCM, yielding the title compound as a solid (8.24 g). Method LCMS1: Rt=0.47 min; [M+H]⁺=326.

### Step 4: 2-(4-(2,4-dioxotetrahydropyrimidin-1(2H)-yl)phenoxy)acetic acid

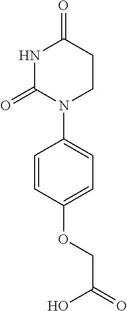 [SMILES: O=C(O)COc1ccc(N2CCC(=O)NC2=O)cc1]

To a 250 mL round bottom flask were added 3,3′-((4-(2-methoxy-2-oxoethoxy)phenyl)azanediyl)dipropionic acid (8.24 g, 25.09 mmol), urea (2.26 g, 37.6 mmol) and HOAc (60 mL). The RM was stirred at 120° C. overnight, an aq. solution of HCl (4 M, 80 mL) was added and the RM was stirred at 120° C. for 45 min. The RM was cooled to 0° C. and filtered, yielding the title compound as a solid (4.93 g). Method LCMS1: Rt=0.75 min; [M+H]⁺=265.

### Step 5: tert-Butyl 4-((1-(2-(4-(2,4-dioxotetrahydropyrimidin-1(2H)-yl)phenoxy)acetyl)piperidin-4-yl)oxy)piperidine-1-carboxylate

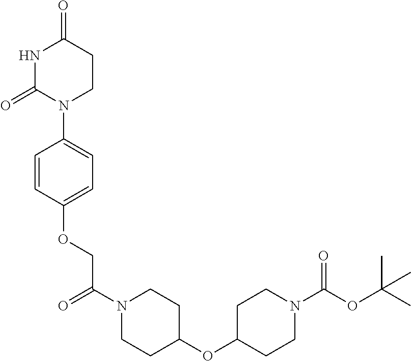 [SMILES: CC(C)(C)OC(=O)N1CCC(OC2CCN(C(=O)COc3ccc(N4CCC(=O)NC4=O)cc3)CC2)CC1]

To a 50 mL round bottom flask were added tert-butyl 4-(piperidin-4-yloxy)piperidine-1-carboxylate (intermediate A, 538 mg, 1.892 mmol), 2-(4-(2,4-dioxotetrahydropyrimidin-1(2H)-yl)phenoxy)acetic acid (500 mg, 1.892 mmol), HATU (863 mg, 2.271 mmol), TEA (0.800 mL, 5.74 mmol) and DMF (8 mL). The RM was stirred at RT for 6 h. The mixture was diluted with EtOAc and water, the aqueous layer was extracted with EtOAc, the combined organic phases were washed with brine and dried over MgSO₄, yielding the title compound as a solid (795 mg), which was directly used for next step without further purification. Method LCMS1: Rt=0.90 min; [M+H]⁺=531.

### Step 6: 1-(4-(2-oxo-2-(4-(piperidin-4-yloxy)piperidin-1-yl)ethoxy)phenyl)dihydropyrimidine-2,4(1H,3H)-dione

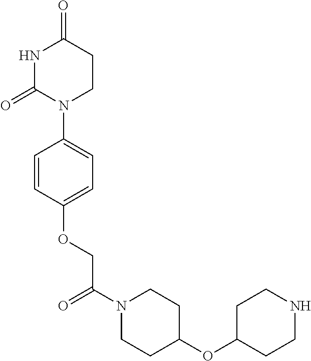 [SMILES: O=C1CCN(c2ccc(OCC(=O)N3CCC(OC4CCNCC4)CC3)cc2)C(=O)N1]

To a 25 mL round bottom flask were added tert-butyl 4-((1-(2-(4-(2,4-dioxotetrahydropyrimidin-1(2H)-yl)phenoxy)acetyl)piperidin-4-yl)oxy)piperidine-1-carboxylate (795 mg, 1.423 mmol), a solution of HCl (4 M) in 1,4-dioxane (10 mL, 40.0 mmol), MeOH (5 mL), and DCM (5 mL). The RM was stirred at RT for 1 h, concentrated, diluted with water and freeze dried, yielding the corresponding HCl salt of the title compound as a solid (785 mg). Method LCMS1: Rt=0.43 min; [M+H]⁺=431.

### ILB-56: 1-(2-methoxy-5-(3,9-diazaspiro[5.5]undecane-3-carbonyl)phenyl)dihydropyrimidine-2,4(1H,3H)-dione

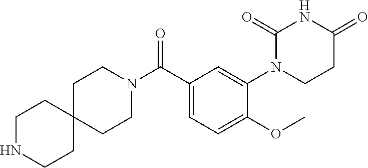 [SMILES: COc1ccc(C(=O)N2CCC3(CCNCC3)CC2)cc1N1CCC(=O)NC1=O]

This compound was prepared as described in PCT/IB2019/052346 intermediate 21.

### ILB-57: 1-(2-methoxy-5-(1-oxa-4,9-diazaspiro[5.5]undecane-4-carbonyl)phenyl)dihydropyrimidine-2,4(1H,3H)-dione

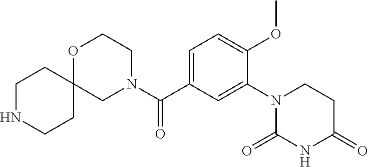 [SMILES: COc1ccc(C(=O)N2CCOC3(CCNCC3)C2)cc1N1CCC(=O)NC1=O]

### Step 1: tert-Butyl 4-(3-(2,4-dioxotetrahydropyrimidin-1(2H)-yl)-4-methoxybenzoyl)-1-oxa-4,9-diazaspiro[5.5]undecane-9-carboxylate

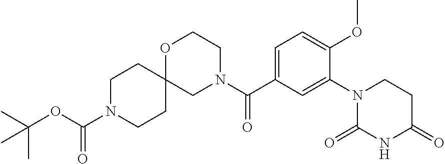 [SMILES: COc1ccc(C(=O)N2CCOC3(CCN(C(=O)OC(C)(C)C)CC3)C2)cc1N1CCC(=O)NC1=O]

A mixture of tert-butyl 1-oxa-4,9-diazaspiro[5.5]undecane-9-carboxylate (CAS No. [930785-40-3], 256 mg, 1 mmol), 3-(2,4-dioxotetrahydropyrimidin-1(2H)-yl)-4-methoxybenzoic acid (ILB-26, 264 mg, 1 mmol) and K₂CO₃ (276 mg, 2 mmol) in DMF (6 mL) was stirred at RT, then HATU (570 mg, 1.5 mmol) was added and the mixture was stirred at RT for 2 h. After filtration, the mixture was purified by reverse phase chromatography using Method PA on a Xtimate® C18 column eluting with ACN in an aq. solution of TFA (0.1%) to afford the title compound as a yellow solid (450 mg). Method XF: Rt=1.16 min; [M-Boc+H]⁺=403.

### Step 2: 1-(2-methoxy-5-(1-oxa-4,9-diazaspiro[5.5]undecane-4-carbonyl)phenyl)dihydropyrimidine-2,4(1H,3H)-dione

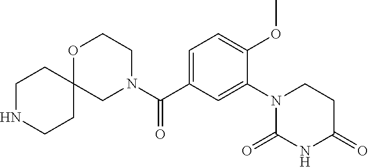 [SMILES: COc1ccc(C(=O)N2CCOC3(CCNCC3)C2)cc1N1CCC(=O)NC1=O]

A mixture of tert-butyl 4-(3-(2,4-dioxotetrahydropyrimidin-1(2H)-yl)-4-methoxybenzoyl)-1-oxa-4,9-diazaspiro[5.5]undecane-9-carboxylate (450 mg, 0.9 mmol) in DCM (3 mL) was stirred at RT. Then, TFA (3 mL) was added and the mixture was stirred at RT for 16 h. The solvent was removed in vacuo to afford the title compound as a yellow solid TFA salt (760 mg). Method XF: Rt=0.72 min; [M+H]⁺=403.

### ILB-58: 1-(4-(2-(3,9-diazaspiro[5.5]undecan-3-yl)ethoxy)phenyl)dihydropyrimidine-2,4(1H,3H)-dione

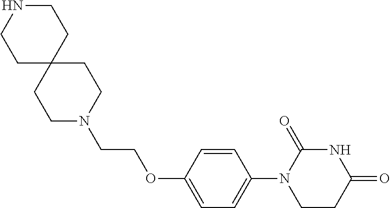 [SMILES: O=C1CCN(c2ccc(OCCN3CCC4(CCNCC4)CC3)cc2)C(=O)N1]

### Step 1: 2-(4-(2,4-dioxotetrahydropyrimidin-1(2H)-yl)phenoxy)acetaldehyde

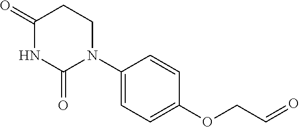 [SMILES: O=CCOc1ccc(N2CCC(=O)NC2=O)cc1]

To a solution of 1-(4-(2,2-dimethoxyethoxy)phenyl)dihydropyrimidine-2,4(1H,3H)-dione (ILB-21, 190 mg, 0.646 mmol) in acetone (2 mL) was added 2M HCl (1.6 mL, 3.23 mmol). The RM was stirred at 50° C. for 18 h. The precipitate was collected by filtration and dried under high vacuum for 18 h to afford the title compound (91 mg). Method XP-A: Rt=046 min; [M+H]⁺=249.2.

### Step 2: tert-Butyl 9-(2-(4-(2,4-dioxotetrahydropyrimidin-1(2H)-yl)phenoxy)ethyl)-3,9-diazaspiro[5.5]undecane-3-carboxylate

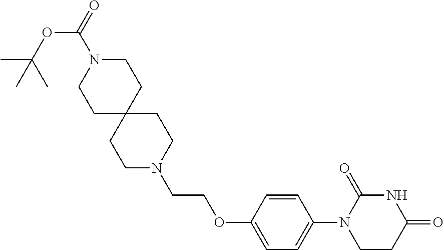 [SMILES: CC(C)(C)OC(=O)N1CCC2(CCN(CCOc3ccc(N4CCC(=O)NC4=O)cc3)CC2)CC1]

To a white mixture of 2-(4-(2,4-dioxotetrahydropyrimidin-1(2H)-yl)phenoxy)acetaldehyde (100 mg, 0.403 mmol) and tert-butyl 3,9-diazaspiro[5.5]undecane-3-carboxylate (CAS No. [173405-78-2], 113 mg, 0.443 mmol) in MeOH (8 mL) was added ZnCl₂ 0.5 M in THF (0.886 mL, 0.443 mmol). The resulting white mixture was flushed with N₂ and stirred at RT for 4.5 h. Then, NaBH₃CN (40 mg, 0.604 mmol) was added, the RM was stirred at RT for 18 h, diluted with ACN and concentrated to afford a white resin. The resin was dissolved in MeOH/ACN, adsorbed on Isolute®, concentrated, and purified by reverse phase chromatography on a Redisep® C18 column eluting with ACN in an aq. solution of TFA (0.1%) to afford, after freeze drying, the title compound as a white solid TFA salt (229 mg). Method LCMS1: Rt=0.69 min; [M+H]⁺=487.4.

### Step 3: 1-(4-(2-(3,9-diazaspiro[5.5]undecan-3-yl)ethoxy)phenyl)dihydropyrimidine-2,4(1H,3H)-dione

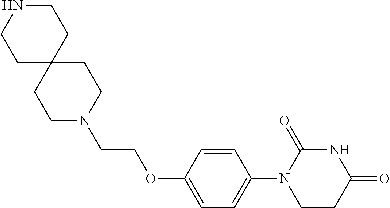 [SMILES: O=C1CCN(c2ccc(OCCN3CCC4(CCNCC4)CC3)cc2)C(=O)N1]

To a colorless solution of tert-butyl 9-(2-(4-(2,4-dioxotetrahydropyrimidin-1(2H)-yl)phenoxy)ethyl)-3,9-diazaspiro[5.5]undecane-3-carboxylate (228 mg, 0.380 mmol) in DCM (5.4 mL) was added TFA (877 μL, 11.39 mmol). The resulting solution was stirred at RT for 1 h, diluted with DCM, concentrated until dryness, then co-evaporated with DCM, dried under HV pump and freeze dried to afford the title compound as an off-white solid TFA salt (266 mg). Method LCMS1: Rt=0.19 min; [M+H]⁺=387.3.

### ILB-59: 1-(2-methoxy-5-(4-(piperidin-4-yloxy)piperidine-1-carbonyl)phenyl)dihydropyrimidine-2,4(1H,3H)-dione

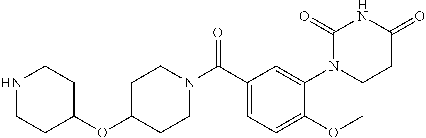 [SMILES: COc1ccc(C(=O)N2CCC(OC3CCNCC3)CC2)cc1N1CCC(=O)NC1=O]

This compound was prepared as described in PCT/IB2019/052346 compound 32, step 2.

### ILB-60: 1-(2-Chloro-5-(4-(piperidin-4-yloxy)piperidine-1-carbonyl)phenyl)dihydropyrimidine-2,4(1H,3H)-dione

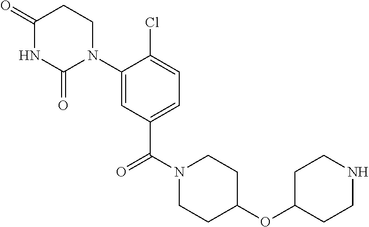 [SMILES: O=C1CCN(c2cc(C(=O)N3CCC(OC4CCNCC4)CC3)ccc2Cl)C(=O)N1]

### Step 1: tert-Butyl 4-((1-(4-chloro-3-(2,4-dioxotetrahydropyrimidin-1(2H)-yl)benzoyl)piperidin-4-yl)oxy)piperidine-1-carboxylate

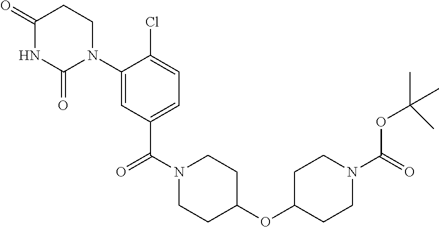 [SMILES: CC(C)(C)OC(=O)N1CCC(OC2CCN(C(=O)c3ccc(Cl)c(N4CCC(=O)NC4=O)c3)CC2)CC1]

To a 50 mL round bottom flask were added HATU (849 mg, 2.233 mmol), 4-chloro-3-(2,4-dioxotetrahydropyrimidin-1(2H)-yl)benzoic acid (ILB-25, 500 mg, 1.861 mmol), DIPEA (1 mL, 5.73 mmol) and DMF (10 mL). The RM was stirred at RT for 30 min, solid tert-butyl 4-(piperidin-4-yloxy)piperidine-1-carboxylate (CAS No. [845305-83-1], 529 mg, 1.861 mmol) was added and the RM was stirred at RT for 1.5 h. The solvent was removed and the residue was purified by reversed phase chromatography on a RediSep® Gold HP C18 column (50 g) eluting with ACN (from 2% to 100%) in an aq. solution of TFA (0.1%), yielding the title compound as a solid (1.07 g). Method LCMS1: Rt=0.98 min; [M+H]⁺=535.

### Step 2: 1-(2-chloro-5-(4-(piperidin-4-yloxy)piperidine-1-carbonyl)phenyl)dihydropyrimidine-2,4(1H,3H)-dione

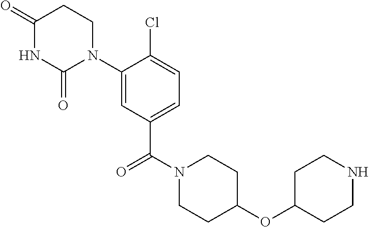 [SMILES: O=C1CCN(c2cc(C(=O)N3CCC(OC4CCNCC4)CC3)ccc2Cl)C(=O)N1]

To a 25 mL round bottom as were added tert-butyl 4-((1-(4-chloro-3-(2,4-dioxotetrahydropyrimidin-1(2H)-yl)benzoyl)piperidin-4-yl)oxy)piperidine-1-carboxylate (1.07 g, 1.820 mmol), a solution of HCl (4 M) in 1,4-dioxane (9 mL) and 1,4-dioxane (9 mL). The RM was stirred at RT for 3 h, the solvents were removed, the residue was redissolved in a mixture of water and ACN and freeze dried, yielding the corresponding hydrochloride salt of the title compound as a solid (884 mg). Method LCMS1: Rt=0.47 min; [M+H]⁺=435.

### ILB-61: 1-(2-chloro-4-(2,2-diethoxyethoxy)phenyl)dihydropyrimidine-2,4(1H,3H)-dione

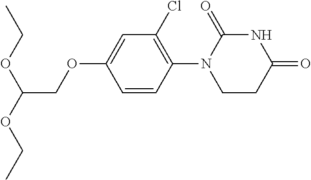 [SMILES: CCOC(COc1ccc(N2CCC(=O)NC2=O)c(Cl)c1)OCC]

To a mixture of 1-(2-chloro-4-hydroxyphenyl)dihydropyrimidine-2,4(1H,3H)-dione (ILB-12, 1.69 g, 7.02 mmol) in DMF (18 mL) was added potassium carbonate (4.37 g, 31.6 mmol) and potassium iodide (0.583 g, 3.51 mmol) at RT. The RM was stirred for 0.5 h at RT. Afterwards 2-bromo-1,1-diethoxyethane (1.091 mL, 7.02 mmol) was added and the RM was heated overnight at 80° C. UPLC-MS next morning showed the reaction was incomplete. Again 2-bromo-1,1-diethoxyethane (1.091 mL, 7.02 mmol) was added and stirred overnight at 80° C. then standing over the weekend at room temperature. The reaction was stopped. The RM was diluted with AcOEt. The organic phase was washed with water and brine. The organic phase was dried over Na₂SO₄, filtered, and concentrated in vacuo to obtain the crude material: brown oil. The crude material was purified by flash chromatography on a silica flash column 24 g eluting with DCM/MeOH to afford the title compound (0.99 g). Method LCMS1: Rt=0.87 min; [M+NH₃]⁺=374.2.

### ILB-62: 1-(4-(2,2-diethoxyethoxy)-2-methylphenyl)dihydropyrimidine-2,4(1H,3H)-dione

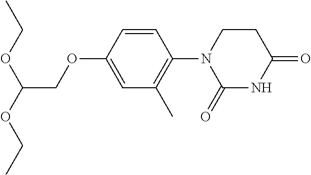 [SMILES: CCOC(COc1ccc(N2CCC(=O)NC2=O)c(C)c1)OCC]

To a mixture of 1-(4-hydroxy-2-methylphenyl)dihydropyrimidine-2,4(1H,3H)-dione (ILB-13, 0.95 g, 4.31 mmol) in DMF (10 mL) was added potassium carbonate (2.68 g, 19.41 mmol) and potassium iodide (0.358 g, 2.157 mmol) at room temperature. The RM was stirred for 0.5 h at RT. Afterwards 2-bromo-1,1-diethoxyethane (0.670 mL, 4.31 mmol) was added and the RM was heated overnight at 80° C. Again 2-bromo-1,1-diethoxyethane (0.670 mL, 4.31 mmol) was added and stirred overnight at 80° C. The RM was diluted with AcOEt. The organic phase was washed with water and brine. The organic phase was dried over Na₂SO₄, filtered, and concentrated in vacuo to obtain the crude material: 1.68 g brown oil. The RM was concentrated and absorbed on silica gel and purified by flash chromatography on a silica flash column 24 g eluting with DCM/MeOH to afford the title compound (0.52 g). Method LCMS1: Rt=0.83 min; [M+H₂O]⁺=354.2.

### ILB-63: 1-(3-(3-(1-oxa-4,9-diazaspiro[5.5]undecan-4-yl)prop-1-yn-1-yl)phenyl)dihydropyrimidine-2,4(1H,3H)-dione

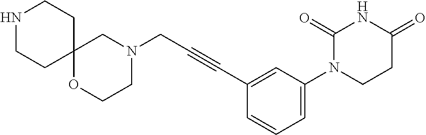 [SMILES: O=C1CCN(c2cccc(C#CCN3CCOC4(CCNCC4)C3)c2)C(=O)N1]

### Step 1: tert-butyl 4-(3-(3-(2,4-dioxotetrahydropyrimidin-1(2H)-yl)phenyl)prop-2-yn-1-yl)-1-oxa-4,9-diazaspiro[5.5]undecane-9-carboxylate

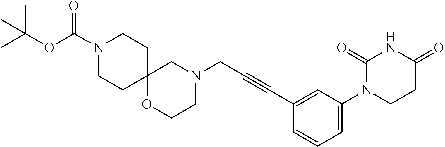 [SMILES: CC(C)(C)OC(=O)N1CCC2(CC1)CN(CC#Cc1cccc(N3CCC(=O)NC3=O)c1)CCO2]

A mixture of Intermediate EE (500 mg, 1.58 mmol), Intermediate DD (559 mg, 1.90 mmol), Pd(PPh₃)₂Cl₂ (56 mg, 0.08 mmol), CuI (30 mg, 0.16 mmol), and Et₃N (0.7 mL, 4.75 mmol) in DMF (5 mL) was stirred at 60° C. for 16 h. The reaction mixture was poured into 50 mL H₂O, extracted with EtOAc (2×50 mL), washed with water, brine, dried, and concentrated. The crude residue was purified by silica gel column chromatography (0% to 60% EtOAc in PE) to give the desired product as light yellow solid (500 mg). Method G: Rt=1.86 min, [M-Boc+H]⁺=383.

### Step 2: 1-(3-(3-(1-oxa-4,9-diazaspiro[5.5]undecan-4-yl)prop-1-yn-1-yl)phenyl)dihydropyrimidine-2,4(1H,3H)-dione

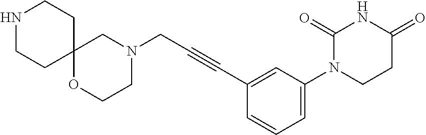 [SMILES: O=C1CCN(c2cccc(C#CCN3CCOC4(CCNCC4)C3)c2)C(=O)N1]

tert-Butyl 4-(3-(3-(2,4-dioxotetrahydropyrimidin-1(2H)-yl)phenyl)prop-2-yn-1-yl)-1-oxa-4,9-diazaspiro[5.5]undecane-9-carboxylate (Step 1, 500 mg, 1.04 mmol) was dissolved in TFA/DCM (3 mL/1 mL) at RT and the mixture was stirred at RT for 16 h. The mixture was concentrated to give 500 mg of the desired product as dark brown gummy oil. Method G: Rt=1.41 min, [M+H]⁺=383

### ILB-64: 1-(3-(4-(4-(piperazine-1-carbonyl)piperazin-1-yl)but-1-yn-1-yl)phenyl)dihydropyrimidine-2,4(1H,3H)-dione

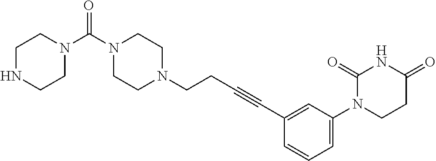 [SMILES: O=C1CCN(c2cccc(C#CCCN3CCN(C(=O)N4CCNCC4)CC3)c2)C(=O)N1]

### Step 1: 3-((3-iodophenyl)amino)propanoic acid

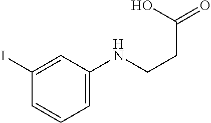 [SMILES: O=C(O)CCNc1cccc(I)c1]

To a solution of 3-iodoaniline (10 g, 45.66 mmol) in toluene (131 mL) was added acrylic acid (4.28 g, 59.36 mmol). The mixture was stirred at 115° C. for 48 h. The solvent was removed to obtain the title compound as an orange oil (15 g, crude). Method H: Rt=1.36 min; [M+H]⁺=291.9.

### Step 2: 1-(3-iodophenyl)dihydropyrimidine-2,4(1H,3H)-dione

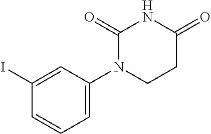 [SMILES: O=C1CCN(c2cccc(I)c2)C(=O)N1]

To a solution of 3-((3-iodophenyl)amino)propanoic acid (15 g, 51.5 mmol) in AcOH (125 mL) was added urea (9.284 g, 154.6 mmol). The mixture was stirred at 120° C. for 16 h. The solvent was removed and water (200 mL) was added. The mixture was filtered. The filter cake was washed with water (2×20 mL) and dried under vacuum. The solid was suspended in EtOAc (60 mL), triturated for 16 h at RT. The mixture was filtered. The filter cake was washed with EtOAc (2×5 mL) and dried to afford the title compound as a pale solid (7.9 g). Method E: Rt=1.43 min; [M+H]⁺=317.0.

### Step 3: tert-Butyl 4-(4-(but-3-yn-1-yl)piperazine-1-carbonyl)piperazine-1-carboxylate

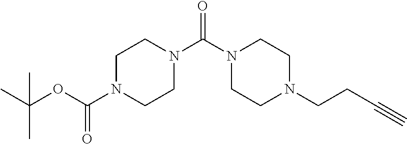 [SMILES: C#CCCN1CCN(C(=O)N2CCN(C(=O)OC(C)(C)C)CC2)CC1]

To a suspension of 1-(but-3-yn-1-yl)piperazine dihydrochloride (3.86 g, 17.36 mmol) in DCM (54 mL) flushed with argon was added, at RT, TEA (9.82 mL, 70.5 mmol). Then, a solution of 4-Boc-1-piperazinecarbonyl chloride in DMF (18 mL) was added slowly (exothermic), and the light brown RM was stirred at RT for 2 days. The RM was diluted with DCM (25-30 mL) and water (90 mL) and the layers were separated. The aq. layer was extracted with EtOAc (60 mL). The combined organic layers were dried over MgSO₄, filtered, and concentrated under HV to give the title compound as a yellow-beige solid (6.23 g). Method LCMS1: Rt=0.59 min; [M+H]⁺=351.1.

### Step 4: tert-Butyl 4-(4-(4-(3-(2,4-dioxotetrahydropyrimidin-1(2H)-yl)phenyl)but-3-yn-1-yl)piperazine-1-carbonyl)piperazine-1-carboxylate

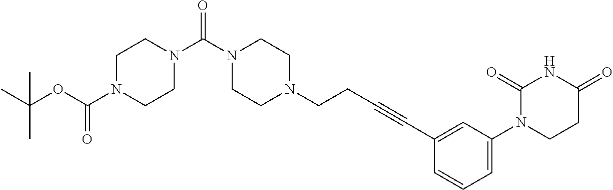 [SMILES: CC(C)(C)OC(=O)N1CCN(C(=O)N2CCN(CCC#Cc3cccc(N4CCC(=O)NC4=O)c3)CC2)CC1]

To a solution of tert-butyl 4-(4-(but-3-yn-1-yl)piperazine-1-carbonyl)piperazine-1-carboxylate (420 mg, 1.2 mmol) in DMF (3 mL) was added 1-(3-iodophenyl)dihydropyrimidine-2,4(1H,3H)-dione (316 mg, 1 mmol), CuI (38 mg, 0.2 mmol) and TEA (607 mg, 6 mmol) at RT. The RM was degassed by bubbling N₂ through the mixture for 5 min then Pd(PPh₃)₂Cl₂ (70 mg, 0.1 mmol) was added. The RM was stirred at 45° C. for 1 h. The RM was poured into 100 mL water and filtered. The crude filter cake was dried then purified by chromatography on a 25 g silica gel Biotage® column eluting with MeOH in DCM (from 0 to 10%) over 30 min to afford the title compound as a grey foam (350 mg). Method H: Rt=1.76 min; [M+H]⁺=539.

### Step 5: 1-(3-(4-(4-(piperazine-1-carbonyl)piperazin-1-yl)but-1-yn-1-yl)phenyl)dihydropyrimidine-2,4(1H,3H)-dione

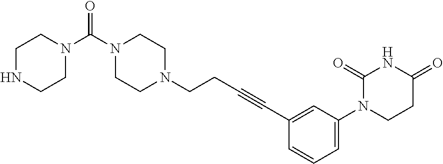 [SMILES: O=C1CCN(c2cccc(C#CCCN3CCN(C(=O)N4CCNCC4)CC3)c2)C(=O)N1]

A solution of tert-butyl 4-(4-(4-(3-(2,4-dioxotetrahydropyrimidin-1(2H)-yl)phenyl)but-3-yn-1-yl)piperazine-1-carbonyl)piperazine-1-carboxylate (265 mg, 0.492 mmol) in DCM (5 mL) and TFA (1 mL) was stirred at RT for 3 h. The solvent was removed. MTBE (10 mL) was added and the mixture concentrated (repeated 3 times) to obtain the title compound as a grey semi-solid TFA salt (300 mg, crude). Method H:Rt=1.357 min; [M+H]⁺=439.

### ILB-65: 2-(4-(2,4-Dioxotetrahydropyrimidin-1(2H)-yl)-1H-pyrazol-1-yl)acetic acid

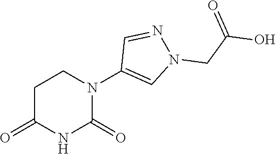 [SMILES: O=C(O)Cn1cc(N2CCC(=O)NC2=O)cn1]

### Step 1: tert-Butyl 2-(4-nitro-1H-pyrazol-1-yl)acetate

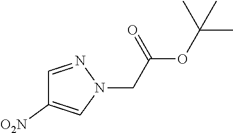 [SMILES: CC(C)(C)OC(=O)Cn1cc([N+](=O)[O-])cn1]

To a solution of 4-nitro-1H-pyrazole (10 g, 88.4 mmol) in ACN (100 mL) at 25° C. was added cesium carbonate (8 mL, 8 mmol) and tert-butyl 2-bromoacetate (14.3 mL, 17.3 g, 88.4 mmol) and the reaction mixture was heated at 80° C. for 5 h. The RM was poured into water and extracted with EtOAc (4×50 mL). The organic phase was washed brine (2×50 mL), dried over Na₂SO₄ and concentrated in vacuo to give crude tert-butyl 2-(4-nitro-1H-pyrazol-1-yl)acetate as a brown solid (21 g) which was used directly in step 2 without further purification. LCMS10: Rt=1.006 min; [M+H−56]⁺=172.

### Step 2: tert-Butyl 2-(4-amino-1H-pyrazol-1-yl)acetate

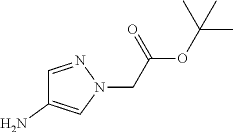

To a solution of crude tert-butyl 2-(4-nitro-1H-pyrazol-1-yl)acetate (21 g) in MeOH (100 mL) at 25° C. was added iron (51.6 g, 924 mmol) and a saturated solution of ammonium chloride in water (100 mL). The reaction mixture was heated at 80° C. for 2 h. The RM was cooled to RT, filtered through a pad of Celite® and the cake was washed with EtOAc (5×70 mL). The filtrate was washed with brine (2×50 mL), dried over Na₂SO₄ and concentrated in vacuo to give a crude oil (12 g). The crude was purified by flash chromatography on silica gel eluting with 50-66% EtOAc in PE to afford tert-butyl 2-(4-amino-1H-pyrazol-1-yl)acetate as a black oil (7 g). LCMS10: Rt=0.329 min; [M+H−56]⁺=142.

### Step 3: 3-((1-(Carboxymethyl)-1H-pyrazol-4-yl)amino)propanoic acid

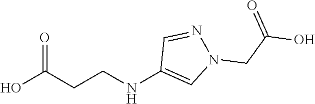 [SMILES: O=C(O)CCNc1cnn(CC(=O)O)c1]

To a solution of tert-butyl 2-(4-amino-1H-pyrazol-1-yl)acetate (5 g, 25.4 mmol) in H₂O (50 mL) at 25° C. was added acrylic acid (2.26 mL, 33.0 mmol). The reaction mixture was heated at 70° C. for 4 h then cooled to RT. The RM was concentrated in vacuo to give crude 3-((1-(carboxymethyl)-1H-pyrazol-4-yl)amino)propanoic acid as a black solid (6.8 g) which was used directly for step 4 without further purification. LCMS10: Rt=0.141 min; [M+H]⁺=214.

### Step 4: 2-(4-(2,4-Dioxotetrahydropyrimidin-1(2H)-yl)-1H-pyrazol-1-yl)acetic acid

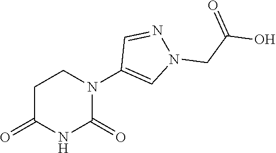 [SMILES: O=C(O)Cn1cc(N2CCC(=O)NC2=O)cn1]

To a solution of crude 3-((1-(carboxymethyl)-1H-pyrazol-4-yl)amino)propanoic acid (6.8 g) in AcOH (50 mL) at 25° C. was added urea (4.55 g, 75.8 mmol). The reaction mixture was heated at 100° C. for 12 h, then cooled to RT. The RM was concentrated in vacuo. EtOH (10 ml) and PE (10 ml) were then added and the mixture stirred at RT. The solid was collected by filtration and washed with cooled PE to afford a crude batch (3 g) which was combined with an additional batch (2.1 g), made under similar conditions, prior to further purification. Thus, the combined filter cake was triturated in MeOH and the solid again collected by filtration. To the black filter cake (2.3 g) was added EtOH (5 mL) and the RM was heated at 80° C. for 2 h. The hot RM was filtered and the filter cake was dried in vacuo providing solid material (1.7 g). An aliquot of this material (300 mg) was purified by reverse phase chromatography on a Water Atlantis® T3C column eluting with ACN in an aq. solution of TFA (0.1%) (from 1% to 25%) Method PA3 to afford 2-(4-(2,4-dioxotetrahydropyrimidin-1(2H)-yl)-1H-pyrazol-1-yl)acetic acid as a white solid (127 mg). LCMS13: Rt=2.558 min; [M+H]⁺=239. ¹H NMR (400 MHz, DMSO-d₆) δ 10.39 (s, 1H), 7.95 (s, 1H), 7.61 (s, 1H), 4.92 (s, 2H), 3.77 (t, J=6.8 Hz, 2H), 2.73-2.67 (m, 2H).

### ILB-66: 3-(4-(2,4-Dioxotetrahydropyrimidin-1(2H)-yl)-1H-pyrazol-1-yl)propanoic acid

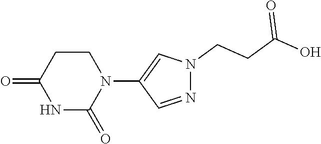 [SMILES: O=C(O)CCn1cc(N2CCC(=O)NC2=O)cn1]

### Step 1: tert-Butyl 3-(4-nitro-1H-pyrazol-1-yl)propanoate

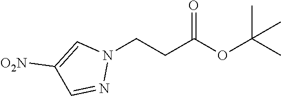 [SMILES: CC(C)(C)OC(=O)CCn1cc([N+](=O)[O-])cn1]

To a solution of 4-nitro-1H-pyrazole (5 g, 44.2 mmol) in DMF (50 mL) at 25° C. was added tert-butyl acrylate (5.67 g, 44.2 mmol) and DBU (10.09 g, 9.9 mL, 66.33 mmol). The reaction mixture was heated at 50° C. for 12 h, then cooled to RT. The RM was poured into water (300 mL) and extracted with ethyl acetate (3×100 mL). The combined organic phase was washed with brine (2×50 mL), dried over Na₂SO₄, filtered and concentrated under reduced pressure to afford the desired product (11 g) as a black oil, which was used directly in Step 2 without further purification. LCMS10: Rt=1.05 min, (M+H−56)⁺=186.0

### Step 2: tert-Butyl 3-(4-amino-1H-pyrazol-1-yl)propanoate

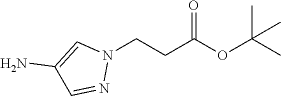

To a solution of tert-butyl 3-(4-nitro-1H-pyrazol-1-yl)propanoate (11 g) in methanol (80 mL), was added iron powder (25.46 g, 456 mmol) and saturated aqueous NH₄Cl (80 mL) at 25° C. The reaction mixture was then heated and stirred at 80° C. for 2 hrs. The mixture was cooled to RT, filtered through a pad of Celite and the cake was washed with ethyl acetate (4×100 mL). The combined organic phase was washed with brine (2×50 mL), dried over anhydrous Na₂SO₄, filtered and concentrated to afford the crude product (10 g) as black oil which was used directly in the next step. LCMS10: Rt=0.628 min, (M+H−56)⁺=156.0

### Step 3: tert-Butyl 3-(4-(2,4-dioxotetrahydropyrimidin-1(2H)-yl)-1H-pyrazol-1-yl)propanoate

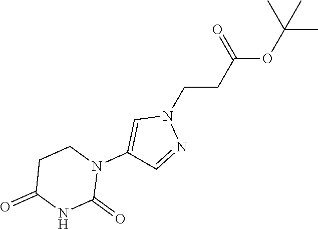 [SMILES: CC(C)(C)OC(=O)CCn1cc(N2CCC(=O)NC2=O)cn1]

To a solution of tert-butyl 3-(4-amino-1H-pyrazol-1-yl)propanoate (10 g) in water (100 mL) was added acrylic acid 3.41 g, 47.3 mmol). The reaction mixture was heated to 70° C. for 4 h, then cooled to RT. The mixture was concentrated under vacuum to afford a black oil (12.5 g), which was then dissolved in acetic acid (100 mL). Urea (7.95 g, 132 mmol) was added and the reaction mixture was heated to 100° C. for 12 h, then cooled and concentrated under vacuum to remove the acetic acid. The residue was purified by flash column chromatography to provide tert-butyl 3-(4-(2,4-dioxotetrahydropyrimidin-1(2H)-yl)-1H-pyrazol-1-yl)propanoate (1.8 g) as a black solid. LCMS10: Rt=0.942 min, (M+H−56)⁺=253.1. ¹H NMR (400 MHz, DMSO-d6) ppm=10.36 (s, 1H), 7.91 (s, 1H), 7.58 (s, 1H), 4.37-4.20 (m, 2H), 3.74 (t, J=6.8 Hz, 2H), 2.80-2.65 (m, 4H), 1.42-1.33 (m, 9H).

### Step 4: 3-(4-(2,4-Dioxotetrahydropyrimidin-1(2H)-yl)-1H-pyrazol-1-yl)propanoic acid

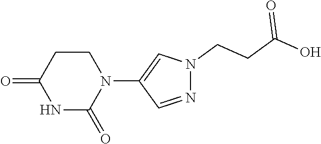 [SMILES: O=C(O)CCn1cc(N2CCC(=O)NC2=O)cn1]

tert-Butyl 3-(4-(2,4-dioxotetrahydropyrimidin-1(2H)-yl)-1H-pyrazol-1-yl)propanoate (1.8 g, 5.6 mmol) was dissolved in DCM (10 mL) and TFA (2 mL) was added. The reaction mixture was stirred at RT for 4 h, then concentrated under vacuum. The solid residue was purified using reverse phase preparative HPLC eluting with ACN in an aq. solution of NH₄CO₃H (10 mM) to give the title compound as a white solid (125 mg). ¹H NMR (400 MHz, DMSO-d6) δ=7.88 (s, 1H), 7.54 (s, 1H), 4.21 (t, J=7.2 Hz, 2H), 3.73 (t, J=6.8 Hz, 2H), 2.68 (t, J=6.8 Hz, 2H), 2.55-2.40 (m, 2H). LCMS Method LCMS10: Rt=0.325 min, (M+H)⁺=253

### ILB-68: 1-(4-((1-(2-aminoethyl)piperidin-4-yl)oxy)phenyl)dihydropyrimidine-2,4(1H,3H)-dione

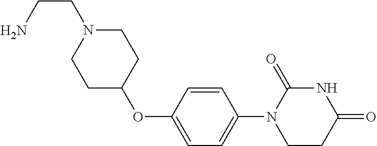 [SMILES: NCCN1CCC(Oc2ccc(N3CCC(=O)NC3=O)cc2)CC1]

### Step 1: tert-Butyl (2-(4-(4-(2,4-dioxotetrahydropyrimidin-1(2H)-yl)phenoxy)piperidin-1-yl)ethyl)carbamate

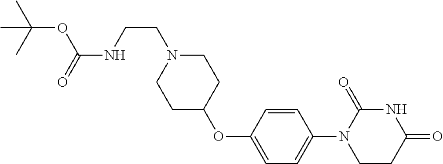 [SMILES: CC(C)(C)OC(=O)NCCN1CCC(Oc2ccc(N3CCC(=O)NC3=O)cc2)CC1]

To a colorless solution of 1-(4-(piperidin-4-yloxy)phenyl)dihydropyrimidine-2,4(1H,3H)-dione (ILB-36, 200 mg, 0.496 mmol), N-Boc-aminoacetaldehyde (CAS No. [89711-08-8], 91 mg, 0.545 mmol), and TEA (69 μL, 0.496 mmol) in MeOH (10 mL) was added ZnCl₂ 0.5 M in THF (1.091 mL, 0.545 mmol). The resulting cloudy solution was flushed with N₂ and stirred at RT. After 3 h at RT, NaBH₃CN (49.2 mg, 0.744 mmol) was added and the RM was stirred at RT for 18 h. More N-Boc-aminoacetaldehyde (25 mg, 0.150 mmol) in MeOH (0.2 mL), followed by more ZnCl₂ 0.5 M in THF (300 μL, 0.150 mmol) were added, the RM was stirred at RT for 2 h, before more NaBH₃CN (23 mg, 0.348 mmol) was added. The resulting RM was then stirred at RT for overnight. The RM was diluted with ACN, concentrated until dryness to give a white resin, that was then redissolved in MeOH/ACN, adsorbed on Isolute®, concentrated until dryness and purified by reverse phase chromatography on a Redisep® C18 column eluting with ACN in an aq. solution of TFA (0.1%) to afford, after freeze drying, the title compound as a white solid TFA salt (169 mg). Method LCMS1: Rt=0.60 min; [M+H]⁺=433.3.

### Step 2: 1-(4-((1-(2-aminoethyl)piperidin-4-yl)oxy)phenyl)dihydropyrimidine-2,4(1H,3H)-dione

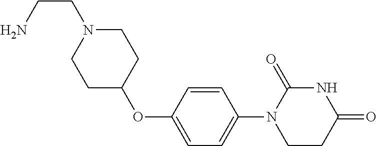 [SMILES: NCCN1CCC(Oc2ccc(N3CCC(=O)NC3=O)cc2)CC1]

To a colorless solution of tert-butyl (2-(4-(4-(2,4-dioxotetrahydropyrimidin-1(2H)-yl)phenoxy)piperidin-1-yl)ethyl)carbamate (166 mg, 0.304 mmol) in DCM (4.35 mL) was added TFA (702 μL, 9.11 mmol). The resulting solution was stirred at RT for 1 h. The RM was diluted with DCM, concentrated until dryness, co-evaporated with DCM (1×), then dried under HV pump and freeze dried to afford the title compound as a white resin TFA salt (188 mg). Method LCMS1: Rt=0.19 min; [M+H]⁺=333.2.

### ILB-69: 3-(3-(((di-tert-butoxyphosphoryl)oxy)methyl)-2,4-dioxotetrahydropyrimidin-1(2H)-yl)-4-methoxybenzoic acid

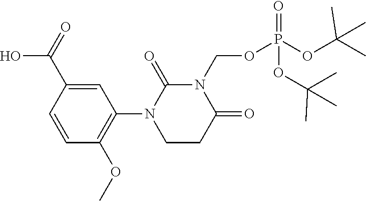 [SMILES: COc1ccc(C(=O)O)cc1N1CCC(=O)N(COP(=O)(OC(C)(C)C)OC(C)(C)C)C1=O]

### Step 1: Benzyl 3-(2,4-dioxotetrahydropyrimidin-1(2H)-yl)-4-methoxybenzoate

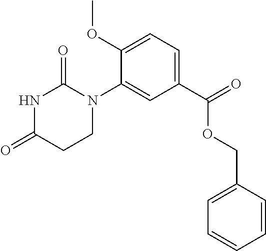 [SMILES: COc1ccc(C(=O)OCc2ccccc2)cc1N1CCC(=O)NC1=O]

To a solution of 3-(2,4-dioxotetrahydropyrimidin-1(2H)-yl)-4-methoxybenzoic acid (ILB-26, 800 mg, 3.03 mmol) in DMF (25 mL) under N₂ at RT were added 4-(dimethylamino)pyridine (CAS No. [1122-58-3], 407 mg, 3.33 mmol), N-(3-Dimethylaminopropyl)-N′-ethylcarbodiimide hydrochloride (CAS No. [25952-53-8], 638 mg, 3.33 mmol) and benzyl alcohol (CAS No. [100-51-6], 0.345 mL, 3.33 mmol). The RM was stirred at RT overnight, then concentrated under vacuum. The residue was purified by flash chromatography on silica gel eluting with 0-100% EtOAc in heptane to afford the title compound as a white powder (730 mg). Method LCMS1: Rt=0.89 min; [M+H]⁺=355.3.

### Step 2: Benzyl 3-(3-(((di-tert-butoxyphosphoryl)oxy)methyl)-2,4-dioxotetrahydropyrimidin-1(2H)-yl)-4-methoxybenzoate

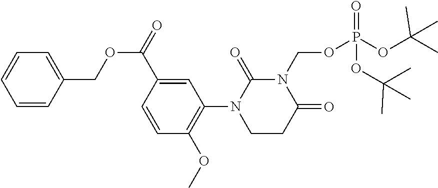 [SMILES: COc1ccc(C(=O)OCc2ccccc2)cc1N1CCC(=O)N(COP(=O)(OC(C)(C)C)OC(C)(C)C)C1=O]

A mixture of benzyl 3-(2,4-dioxotetrahydropyrimidin-1(2H)-yl)-4-methoxybenzoate (100 mg, 0.282 mmol), di-tert-butyl(chloromethyl) phosphate (CAS No. [229625-50-7]) (0.144 mL, 0.621 mmol), Cs₂CO₃ (276 mg, 0.847 mmol) and KI (94 mg, 0.564 mmol) in dry ACN (1 mL) was flushed with N₂ and stirred at RT for 3 nights. The RM was diluted with EtOAc (5 mL), filtered through a frit, rinsed with EtOAc (2×), then the filtrate was concentrated at 35° C. and dried overnight under HV to afford a crude yellow oil. The oil was purified by flash chromatography on silica gel eluting with 30-100% (CHX/acetone 1/1) in CHX to afford the title compound as a white solid (48 mg). Method LCMS1: Rt=1.21 min; [M+H]⁺=577.2.

### Step 3: 3-(3-(((di-tert-butoxyphosphoryl)oxy)methyl)-2,4-dioxotetrahydropyrimidin-1(2H)-yl)-4-methoxybenzoic acid

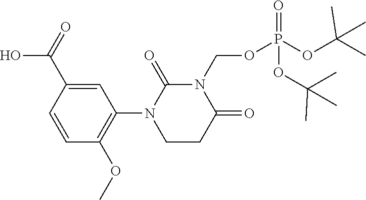 [SMILES: COc1ccc(C(=O)O)cc1N1CCC(=O)N(COP(=O)(OC(C)(C)C)OC(C)(C)C)C1=O]

A colorless solution of benzyl 3-(3-(((di-tert-butoxyphosphoryl)oxy)methyl)-2,4-dioxotetrahydropyrimidin-1(2H)-yl)-4-methoxybenzoate (46 mg, 0.077 mmol) in a mixture of EtOAc (1.28 mL) and EtOH (0.64 mL) was treated with 10% palladium on activated charcoal (CAS No. [7440-05-3]) (8.2 mg, 7.7 μmol) and the resulting black RM was stirred at RT under H₂ atmosphere for 1 h. Then, the RM was diluted with EtOAc/EtOH 2/1 (2 mL), then filtered through Hyflo® and rinsed with EtOAc/EtOH 2/1 (3×2 mL). The filtrate was then concentrated until dryness to afford the title compound as a white solid (40 mg). Method LCMS1: Rt=0.88 min; [M+H]⁺=487.2. ¹H NMR (400 MHz, DMSO-d₆) δ ppm 1.41 (s, 18H) 2.79-3.02 (m, 2H) 3.62 (br t, J=6.28 Hz, 2H) 3.87 (s, 3H) 5.47 (br d, J=5.53 Hz, 2H) 7.22 (d, J=8.83 Hz, 1H) 7.86 (d, J=1.80 Hz, 1H) 7.93 (dd, J=8.60, 2.02 Hz, 1H) 12.83 (s, 1H).

### ILB-71: Ethyl 2-(5-(2,4-dioxotetrahydropyrimidin-1(2H)-yl)pyridin-3-yl)acetate

### ILB-72: Methyl 5-(2,4-dioxotetrahydropyrimidin-1(2H)-yl)nicotinate

### ILB-73: 1-(5-Chloropyridin-3-yl)dihydropyrimidine-2,4(1H,3H)-dione

### ILB-74: 1-(5-(Prop-1-en-2-yl)pyridin-3-yl)dihydropyrimidine-2,4(1H,3H)-dione

### ILB-75: 1-(6-(1-Hydroxyethyl)pyridin-3-yl)dihydropyrimidine-2,4(1H,3H)-dione

### ILB-76: 1-(5-Bromopyridin-3-yl)dihydropyrimidine-2,4(1H,3H)-dione

### ILB-77: 1-(5-Hydroxypyridin-3-yl)dihydropyrimidine-2,4(1H,3H)-dione

### ILB-78: Ethyl 5-(2,4-dioxotetrahydropyrimidin-1(2H)-yl)nicotinate

### ILB-79: 1-(1H-Pyrazol-4-yl)dihydropyrimidine-2,4(1H,3H)-dione

### ILB-80: 1-(5-Bromopyridin-3-yl)dihydropyrimidine-2,4(1H,3H)-dione

Compounds ILB-71 to ILB-80 were prepared in a parallel synthesis format requiring the synthetic intermediates PMB-DHU and DMB-DHU:

### 3-(4-Methoxybenzyl)dihydropyrimidine-2,4(1H,3H)-dione

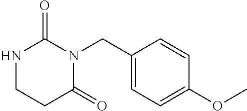 [SMILES: COc1ccc(CN2C(=O)CCNC2=O)cc1]

### Step 1: Dihydropyrimidine-2,4(1H,3H)-dione

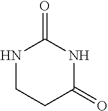

Eight parallel reactions: A mixture of pyrimidine-2,4-diol (25.0 g, 223 mmol) and Rh/C (10.0 g) in H₂O (1.5 L) was degassed under vacuum and purged with H₂ several times. The mixture was stirred at 80° C. for 32 h under H₂ (30 psi). Eight parallel reactions were combined and filtered. The filtrate was concentrated to give crude product which was washed with MeOH (2×200 mL) to give dihydropyrimidine-2,4(1H,3H)-dione (192 g) as a white solid. ¹H NMR (400 MHz, DMSO-d₆) δ=9.93 (br s, 1H), 7.49 (br s, 1H), 3.20 (dt, J=2.1, 6.7 Hz, 2H), 2.43 (t, J=6.7 Hz, 2H).

### Step 2: 3-(4-Methoxybenzyl)dihydropyrimidine-2,4(1H,3H)-dione

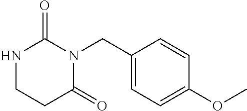 [SMILES: COc1ccc(CN2C(=O)CCNC2=O)cc1]

Three parallel reactions: To a mixture of dihydropyrimidine-2,4(1H,3H)-dione (60.0 g, 525 mmol) and Cs₂CO₃ (257 g, 789 mmol) in DMSO (1.2 L) was added a solution of PMBCl (82.4 g, 526 mmol) in CH₂Cl₂ (500 mL) at 20° C. The mixture was stirred at 20° C. for 15 h. Three parallel reactions were combined and filtered. After addition of brine (6.0 L) to the filtrate, a white precipitate was formed. The solid was collected by filtration and washed with EtOAc (2×300 mL). The aqueous filtrate was then extracted with EtOAc (3×1 L). The combined organic phases were dried over Na₂SO₄, filtered and concentrated to give the crude product which was washed with EtOAc (2×150 mL) to give a white solid. The solid crops were combined and dried to give 3-(4-methoxybenzyl)dihydropyrimidine-2,4(1H,3H)-dione (195 g) as a white solid. ¹H NMR (400 MHz, DMSO-d₆) δ=7.86-7.77 (m, 1H), 7.17 (d, J=8.6 Hz, 2H), 6.84 (d, J=8.6 Hz, 2H), 4.71 (s, 2H), 3.71 (s, 3H), 3.27-3.14 (m, 2H), 2.62 (t, J=6.8 Hz, 2H).

### 3-(2,4-Dimethoxybenzyl)dihydropyrimidine-2,4(1H,3H)-dione

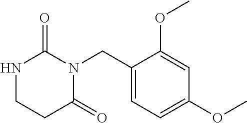 [SMILES: COc1ccc(CN2C(=O)CCNC2=O)c(OC)c1]

### Step 1: tert-Butyl (3-((2,4-dimethoxybenzyl)amino)-3-oxopropyl)carbamate

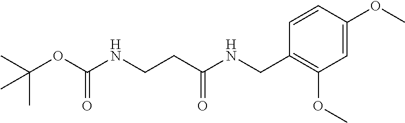 [SMILES: COc1ccc(CNC(=O)CCNC(=O)OC(C)(C)C)c(OC)c1]

To a mixture of 3-((tert-butoxycarbonyl)amino)propanoic acid (400 g, 2.11 mol) in CH₂Cl₂ (2.4 L) was added a suspension of CDI (360 g, 2.22 mol) in CH₂Cl₂ (0.8 L) at 0° C. The mixture was stirred at 0° C. for 30 min. DMBNH₂ (424 g, 2.53 mol) was added to the mixture at 0° C. The mixture was stirred at 25° C. for 1.5 h. The mixture was washed with water (800 mL), sat. aq. Na₂CO₃ (2×800 mL), HCl aq. (2×800 mL) and brine (500 mL). The organic phase was dried over anhydrous Na₂SO₄, filtered, and concentrated in vacuum to give tert-butyl (3-((3,4-dimethoxyphenyl)amino)-3-oxopropyl)carbamate (540 g) as a white solid which was used directly for step 2 without further purification. [M+H]⁺=339.1.

### Step 2: 3-Amino-N-(2,4-dimethoxybenzyl)propanamide hydrochloride

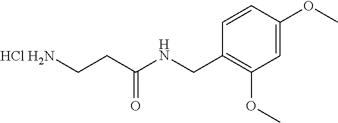

To the mixture of (3-((3,4-dimethoxyphenyl)amino)-3-oxopropyl)carbamate (step 1, 540 g, 1.6 mol) in EtOAc (4 L) was added a solution of HCl in EtOAc (4 mol/L, 1.6 L). The mixture was stirred at 25° C. for 16 h. The mixture was filtrated and the solid was dried under vacuum to give 3-amino-N-(2,4-dimethoxybenzyl)propanamide hydrochloride (430 g) as a white solid which was used directly for step 3 without further purification. ¹H NMR (400 MHz, DMSO-d₆) δ=8.41 (br t, J=5.3 Hz, 1H), 8.29-8.05 (m, 3H), 7.13-7.03 (m, 1H), 6.52 (s, 1H), 6.48-6.43 (m, 1H), 6.40-6.27 (m, 4H), 4.21-4.10 (m, 2H), 3.77 (s, 3H), 3.73 (s, 3H), 3.04-2.88 (m, 2H), 2.58 (s, 2H).

### Step 3: 3-(2,4-Dimethoxybenzyl)dihydropyrimidine-2,4(1H,3H)-dione

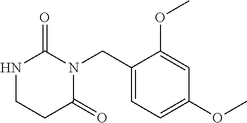 [SMILES: COc1ccc(CN2C(=O)CCNC2=O)c(OC)c1]

To a mixture of 3-amino-N-(2,4-dimethoxybenzyl)propanamide hydrochloride (step 2, 215 g, 0.78 mol) in DCE (2 L) was added DIEA (299 g, 1.56 mol) at 0° C. The mixture was stirred at 0° C. for 0.5 h. Then the mixture was added to a suspension of CDI (152 g, 0.94 mol) in DCE (2 L) and stirred at 25° C. for 16 h. The mixture was warmed to 100° C. and stirred at 100° C. for another 4 h. The mixture was washed with aq. citric acid solution (2×800 mL) and brine (500 mL). The organic phase was dried over anhydrous Na₂SO₄, filtered and concentrated in vacuum to give 3-(2,4-dimethoxybenzyl)dihydropyrimidine-2,4(1H,3H)-dione (170 g, 0.64 mmol) as a white solid. ¹H NMR (400 MHz, DMSO-d₆) δ=7.83 (br s, 1H), 6.83-6.68 (m, 1H), 6.58-6.50 (m, 1H), 6.47-6.34 (m, 1H), 4.74-4.63 (m, 2H), 3.79 (s, 3H), 3.73 (s, 3H), 3.32-3.24 (m, 2H), 2.73-2.62 (m, 2H).

Compounds ILB-71 to ILB-80 were prepared in two steps from either 3-(4-Methoxybenzyl)dihydropyrimidine-2,4(1H,3H)-dione or 3-(2,4-Dimethoxybenzyl)dihydropyrimidine-2,4(1H,3H)-dione in a parallel synthesis format:

### Step 1: PMB-ILB-76: 1-(5-bromopyridin-3-yl)-3-(4-methoxybenzyl)dihydropyrimidine-2,4(1H,3H)-dione PMB-ILB-80: 1-(4-bromothiophen-3-yl)-3-(4-methoxybenzyl)dihydropyrimidine-2,4(1H,3H)-dione PMB-ILB-79: 3-(4-methoxybenzyl)-1-(1H-pyrazol-4-yl)dihydropyrimidine-2,4(1H,3H)-dione

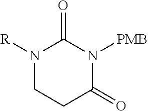

To a mixture of 3-(4-methoxybenzyl)dihydropyrimidine-2,4(1H,3H)-dione (150 mg, 0.64 mmol), heteroaryl bromide (0.83 mmol, 1.3 eq.), K₂CO₃ (266 mg, 3.0 eq.) and DMEDA (6 mg, 0.1 eq.) in DMF (5 mL) was added CuI (12 mg, 0.1 eq.) under N₂. The mixture was stirred at 100° C. for 16 h. To the solution was added thiol resin (500 mg, LS-2000) and the suspension was stirred at RT for 2 h. The suspension was filtered and concentrated to obtain the crude product without Cu catalyst. The crude product was purified by prep-HPLC (HCOOH) to give the desired products.

### PMB-ILB-73: 1-(5-chloropyridin-3-yl)-3-(4-methoxybenzyl)dihydropyrimidine-2,4(1H,3H)-dione

### PMB-ILB-78: Ethyl 5-(3-(4-methoxybenzyl)-2,4-dioxotetrahydropyrimidin-1(2H)-yl)nicotinate

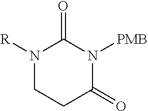

To the mixture of 3-(4-methoxybenzyl)dihydropyrimidine-2,4(1H,3H)-dione (150 mg, 0.64 mmol), heteroaryl bromide (0.83 mol, 1.3 eq.) and K₂CO₃ (266 mg, 1.92 mmol) in DMF (4 mL) was added DMEDA (6 mg, 0.06 mmol) and CuI (12 mg, 0.06 mmol) at 20° C. under N₂. The mixture was stirred at 120° C. for 16 h. To the solution was added thiol resin (500 mg, LS-2000) and the suspension was stirred at 25° C. for 2 h. The suspension was filtered and concentrated to obtain the crude product without Cu catalyst. The crude product was purified by prep-HPLC (HCl or HCOOH) to give the desired products.

### PMB-ILB-77: 1-(5-hydroxypyridin-3-yl)-3-(4-methoxybenzyl)dihydropyrimidine-2,4(1H,3H)-dione

### 1-(5-(2-hydroxypropan-2-yl)pyridin-3-yl)-3-(4-methoxybenzyl)dihydropyrimidine-2,4(1H,3H)-dione

### PMB-ILB-75: 1-(6-(1-hydroxyethyl)pyridin-3-yl)-3-(4-methoxybenzyl)dihydropyrimidine-2,4(1H,3H)-dione

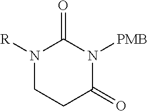

To the mixture of 3-(4-methoxybenzyl)dihydropyrimidine-2,4(1H,3H)-dione (50 mg, 0.21 mmol), heteroaryl bromide (0.19 mol, 0.9 eq.) and K₂CO₃ (89 mg, 0.64 mmol) in DMF (2 mL) was added DMEDA (2 mg, 0.02 mmol) and CuI (4 mg, 0.02 mmol) at 20° C. under N₂. The mixture was stirred at 100° C. for 16 h. To the solution was added thiol resin (500 mg, LS-2000) and the suspension was stirred at 25° C. for 2 h. The suspension was filtered and concentrated to obtain the crude product without Cu catalyst. The crude product was purified by prep-HPLC (HCl or HCOOH) to give the desired products.

### PMB-ILB-72: methyl 5-(3-(4-methoxybenzyl)-2,4-dioxotetrahydropyrimidin-1(2H)-yl)nicotinate

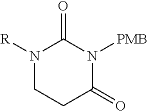

To the mixture of 3-(4-methoxybenzyl)dihydropyrimidine-2,4(1H,3H)-dione (50 mg, 0.21 mmol), heteroaryl bromide (0.19 mmol, 0.9 eq.), KI (43 mg, 1.2 eq.) and K₂CO₃ (89 mg, 0.64 mmol) in DMF (2 mL) was added DMEDA (2 mg, 0.02 mmol) and CuI (4 mg, 0.02 mmol) at 20° C. under N₂. The mixture was stirred at 110° C. for 16 h. To the solution was added thiol resin (500 mg, LS-2000) and the suspension was stirred at 25° C. for 2 h. The suspension was filtered and concentrated to obtain the crude product without Cu catalyst. The crude product was purified by prep-HPLC (HCl or HCOOH) to give the desired product.

### DMB-ILB-71: Ethyl 2-(5-(3-(2,4-dimethoxybenzyl)-2,4-dioxotetrahydropyrimidin-1(2H)-yl)pyridin-3-yl)acetate

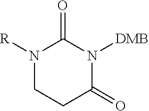

To the mixture of 3-(2,4-dimethoxybenzyl)dihydropyrimidine-2,4(1H,3H)-dione (50 mg, 0.19 mmol), heteroaryl bromide (0.17 mmol, 0.9 eq.), KI (38 mg, 1.2 eq.) and K₂CO₃ (78 mg, 0.58 mmol) in DMF (1 mL) was added DMEDA (2 mg, 0.02 mmol) and CuI (4 mg, 0.02 mmol) at 20° C. under N₂. The mixture was stirred at 100° C. for 16 h. To the solution was added thiol resin (200 mg, LS-2000) and the suspension was stirred at 25° C. for 2 h. The suspension was filtered and concentrated to obtain the crude product without Cu catalyst. The crude product was purified by prep-HPLC (HCl or HCOOH) to give the desired product.

### Step 2: ILB-76: 1-(5-Bromopyridin-3-yl)dihydropyrimidine-2,4(1H,3H)-dione

### ILB-80: 1-(5-Bromopyridin-3-yl)dihydropyrimidine-2,4(1H,3H)-dione

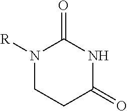

A mixture of either 1-(5-bromopyridin-3-yl)-3-(4-methoxybenzyl)dihydropyrimidine-2,4(1H,3H)-dione or 1-(4-bromothiophen-3-yl)-3-(4-methoxybenzyl)dihydropyrimidine-2,4(1H,3H)-dione (1.0 eq., 0.32 mmol) in TFA/TfOH (1/1, 2 mL) was stirred at 20° C. for 2 h. The mixture was concentrated to give a crude product. The crude product was purified by prep-HPLC (HCOOH) to give the desired product. ILB-76 [M+H]⁺=270.0/272.0. ILB-80 [M+H]⁺=275.0 /277.0.

### ILB-73: 1-(5-Chloropyridin-3-yl)dihydropyrimidine-2,4(1H,3H)-dione

### ILB-78: Ethyl 5-(2,4-dioxotetrahydropyrimidin-1(2H)-yl)nicotinate

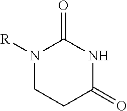

A mixture of 1-(5-chloropyridin-3-yl)-3-(4-methoxybenzyl)dihydropyrimidine-2,4(1H,3H)-dione or ethyl 5-(3-(4-methoxybenzyl)-2,4-dioxotetrahydropyrimidin-1(2H)-yl)nicotinate (1.0 eq., 0.20 mmol) in TFA/TfOH (1/1, 2 mL) was stirred at 20° C. for 16 h. The mixture was concentrated to give a crude product. The crude product was purified by prep-HPLC (HCOOH or HCl) to give the desired product. ILB-73 [M+H]⁺=226.1. ILB-78 [M+H]⁺=264.1.

### ILB-79: 1-(1H-pyrazol-4-yl)dihydropyrimidine-2,4(1H,3H)-dione

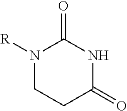

A mixture of 3-(4-methoxybenzyl)-1-(1H-pyrazol-4-yl)dihydropyrimidine-2,4(1H,3H)-dione (0.12 mmol) in TFA/TfOH (5/1, 1 mL) was stirred at 80° C. for 1 h. The mixture was concentrated to give a crude product. The crude product was purified by prep-HPLC (HCl) to give ILB-79. ILB-79 [M+H]⁺=181.0.

### ILB-77: 1-(5-Hydroxypyridin-3-yl)dihydropyrimidine-2,4(1H,3H)-dione

### ILB-74: 1-(5-(Prop-1-en-2-yl)pyridin-3-yl)dihydropyrimidine-2,4(1H,3H)-dione

### ILB-75: 1-(6-(1-Hydroxyethyl)pyridin-3-yl)dihydropyrimidine-2,4(1H,3H)-dione

### ILB-72: Methyl 5-(2,4-dioxotetrahydropyrimidin-1(2H)-yl)nicotinate

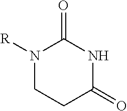

A mixture of either 1-(5-hydroxypyridin-3-yl)-3-(4-methoxybenzyl)dihydropyrimidine-2,4(1H,3H)-dione or 1-(5-(2-hydroxypropan-2-yl)pyridin-3-yl)-3-(4-methoxybenzyl)dihydropyrimidine-2,4(1H,3H)-dione or 1-(6-(1-hydroxyethyl)pyridin-3-yl)-3-(4-methoxybenzyl)dihydropyrimidine-2,4(1H,3H)-dione or methyl 5-(3-(4-methoxybenzyl)-2,4-dioxotetrahydropyrimidin-1(2H)-yl)nicotinate (1.0 eq., 0.10 mmol) in TFA/TfOH (5/1, 1 mL) was stirred at 60° C. for 2 h. The mixture was concentrated to give a crude product. The crude product was purified by prep-HPLC (HCOOH) to give the desired product. ILB-77 [M+H]⁺=208.1. ILB-74 [M+H]⁺=232.1. ILB-75 [M+H]⁺=236.1. ILB-72 [M+H]⁺=250.0.

### ILB-71: Ethyl 2-(5-(2,4-dioxotetrahydropyrimidin-1(2H)-yl)pyridin-3-yl)acetate

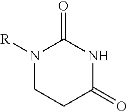

A mixture of ethyl 2-(5-(3-(2,4-dimethoxybenzyl)-2,4-dioxotetrahydropyrimidin-1(2H)-yl)pyridin-3-yl)acetate (1.0 eq., 0.10 mmol) in TFA (1 mL) was stirred at 80° C. for 48 h. The mixture was concentrated to give a crude product. The crude product was purified by prep-HPLC (HCOOH) to give the desired product. ILB-71 [M+H]⁺=278.1.

### ILB-81: 4-(2,4-dioxotetrahydropyrimidin-1(2H)-yl)-3-methoxybenzoic acid

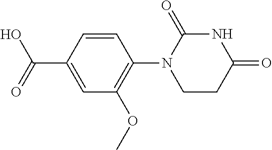 [SMILES: COc1cc(C(=O)O)ccc1N1CCC(=O)NC1=O]

4-amino-3-methoxybenzoic acid (6 g, 35.9 mmol) and acrylic acid (7.5 mL, 108 mmol) were dissolved in acetic acid (23 mL) at RT. H₂SO₄ (11 drops) were added and the resulting brown suspension was stirred at 100° C. overnight. To the RM was added urea (10.8 g, 180 mmol). The resulting brown suspension was stirred at 120° C. for 2 days. The reaction was concentrated to dryness. The crude material was suspended in 10% aqueous HCl, cooled to 0° C. and filtered. The resultant solid was triturated with MBTE and filtered to afford the title compound (541 mg). Method LCMS2: Rt=0.80 min; [M+H]⁺=265.1.

Further material was isolated from the filtrate. The mother liquors were concentrated to dryness and purified by reverse phase chromatography on a RediSep® C18 column eluting with ACN in an aqueous solution of TFA (0.10%) (from 2% to 100%). The appropriate fractions were combined, concentrated to dryness and treated with acetone, observing precipitation. Suspension was filtered and the solid washed with water to afford the title compound as a solid (4.67 g). Method LCMS2: Rt=0.80 min; [M+H]⁺=265.2.

### ILB-82: Ethyl 5-(2,4-dioxotetrahydropyrimidin-1(2H)-yl)-6-methylnicotinate

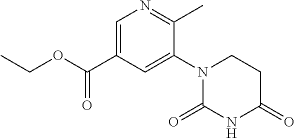 [SMILES: CCOC(=O)c1cnc(C)c(N2CCC(=O)NC2=O)c1]

### Step 1: Ethyl 5-((3-methoxy-3-oxopropyl)amino)-6-methylnicotinate

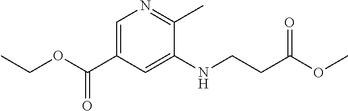 [SMILES: CCOC(=O)c1cnc(C)c(NCCC(=O)OC)c1]

A mixture of ethyl 5-amino-6-methylnicotinate (CAS [1008138-73-5]) (700 mg, 3.88 mmol), methyl acrylate (CAS No. [96-33-3], 1.75 mL, 19.42 mmol) in DMF (1.7 mL) and AcOH (170 μL) was heated in a capped vial at 100° C. for 7 days. The RM was diluted with EtOAc, washed with water (3×) and brine (1×). The organic layer was dried over MgSO₄, filtered and concentrated. The crude product was purified by flash chromatography on silica gel eluting with 20-100% EtOAc in DCM to afford the title compound as a yellow solid (420 mg). Method XO: Rt=1.34 min; [M+H]⁺=267.2.

### Step 2: Ethyl 5-(2,4-dioxotetrahydropyrimidin-1(2H)-yl)-6-methylnicotinate

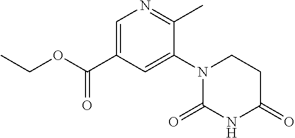 [SMILES: CCOC(=O)c1cnc(C)c(N2CCC(=O)NC2=O)c1]

A 300 mL round bottom flask containing ethyl 5-((3-methoxy-3-oxopropyl)amino)-6-methylnicotinate (600 mg, 2.253 mmol) and a stirring bar was capped and purged with N₂. Then, THF (20 mL) was added, the flask was cooled in an ice bath, and triphosgene (334 mg, 1.127 mmol) was added. To the flask was then added DIPEA (590 μL, 3.38 mmol) portionwise over 5 min. The mixture was stirred at 0° C., then slowly warmed to RT and stirred for 2 h. The flask was cooled in an ice bath, NH₃ 7 N in MeOH (22.53 mL, 158 mmol) was added dropwise, and the mixture was slowly warmed to RT and stirred for overnight. The RM was transferred to a sealed pressure tube and heated overnight at 90° C., then at 100° C. for 24 h. The RM was concentrated under reduced pressure and purified by flash chromatography on silica gel eluting with 5-15% MeOH in DCM to afford a mixture of the title compound and its corresponding methyl ester (500 mg). The mixture was then purified by reverse phase chromatography on a Redisep® C18 column eluting with ACN in an aq. solution of NH₄OH (0.1%) (from 5% to 80%) to afford, after recrystallization from EtOH, the title compound as colorless crystals (80 mg). Method XP: Rt=0.99 min; [M+H]⁺=278.1

### ILB-83: 1-((3-bromothiophen-2-yl)methyl)dihydropyrimidine-2,4(1H,3H)-dione

### ILB-84: 1-((5-(trifluoromethyl)-1H-pyrazol-3-yl)methyl)dihydropyrimidine-2,4(1H,3H)-dione

### ILB-85: 1-((4-bromo-1-methyl-1H-pyrazol-3-yl)methyl)dihydropyrimidine-2,4(1H,3H)-dione

### ILB-87: 2-((2,4-dioxotetrahydropyrimidin-1(2H)-yl)methyl)thiazole-4-carboxylic acid

### ILB-88: 1-((4-bromo-1H-pyrazol-5-yl)methyl)dihydropyrimidine-2,4(1H,3H)-dione

### ILB-89: 1-((3-bromopyridin-4-yl)methyl)dihydropyrimidine-2,4(1H,3H)-dione

### ILB-90: 1-((5-bromopyrimidin-2-yl)methyl)dihydropyrimidine-2,4(1H,3H)-dione

### ILB-91: 1-((4-chloropyridin-2-yl)methyl)dihydropyrimidine-2,4(1H,3H)-dione

### ILB-92: 1-((4-bromopyridin-2-yl)methyl)dihydropyrimidine-2,4(1H,3H)-dione

### ILB-93: 1-((2-amino-5-bromopyridin-3-yl)methyl)dihydropyrimidine-2,4(1H,3H)-dione

Compounds ILB-83 to ILB-93 were prepared in a parallel synthesis format according to a general procedure:

### Step 1: Mixture of carboxyethyl-R-methanamines and bis(carboxyethyl)-R-methanamines

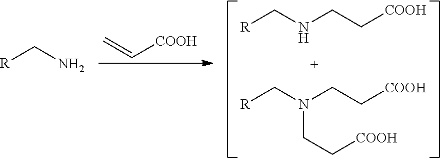

To a solution of amine or its salt (1.0 equiv.) in ACN (2.0 mL), acrylic acid (1.1 or 2.0 equiv.) and TEA (1.1 or 4.0 equiv. per each acid equiv. if amine salt used) were added. The resulting mixture was stirred at 80° C. for 4 h. After the completion of the reaction, the resulting mixture was concentrated under reduced pressure. The obtained crude solid was used in the next step without an additional work-up.

### Step 2: 1-R-methandihydropyrimidine-2,4(1H,3H)-diones

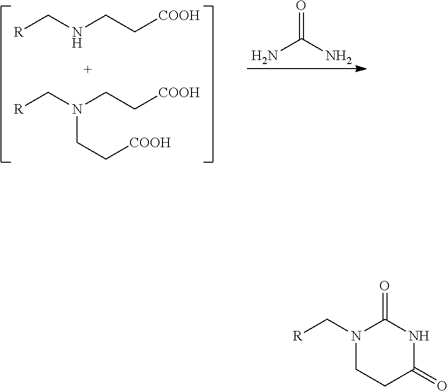

The crude product from step 1 (1.0 equiv.) was dissolved in AcOH (2.0 mL) and urea (3.0 equiv.) was added. After stirring the reaction mixture at 130° C. for 16 h, the solvent was removed in vacuo and the residue was purified by HPLC (C18 column; MeOH—H₂O mobile phase, gradient is given in the tabulated data, run time 5 min) to afford pure product.

### ILB-83: 1-((3-bromothiophen-2-yl)methyl)dihydropyrimidine-2,4(1H,3H)-dione

From (3-bromothiophen-2-yl)methanamine (100.1 mg, 0.52 mmol), acrylic acid (75.1 mg, 1.04 mmol) and urea (93.9 mg, 1.56 mmol); after purification (H₂O, 15-65% MeOH gradient) was obtained 50.6 mg 1-((3-bromothiophen-2-yl)methyl)dihydropyrimidine-2,4(1H,3H)-dione as yellow solid. [M+H]⁺=291.

### ILB-84: 1-((5-(trifluoromethyl)-1H-pyrazol-3-yl)methyl)dihydropyrimidine-2,4(1H,3H)-dione

From (5-(trifluoromethyl)-1H-pyrazol-3-yl)methanamine dihydrochloride (90.8 mg, 0.38 mmol), acrylic acid (30.2 mg, 0.42 mmol), urea (68.7 mg, 1.14 mmol) and TEA (84.9 mg, 0.84 mmol); after purification (H₂O, 10-60% MeOH gradient) was obtained 2.7 mg 1-((5-(trifluoromethyl)-1H-pyrazol-3-yl)methyl)dihydropyrimidine-2,4(1H,3H)-dione as white solid. [M+H]⁺=263.

### ILB-85: 1-((4-bromo-1-methyl-1H-pyrazol-3-yl)methyl)dihydropyrimidine-2,4(1H,3H)-dione

From (4-bromo-1-methyl-1H-pyrazol-3-yl)methanamine (99.7 mg, 0.52 mmol), acrylic acid (75.6 mg, 1.05 mmol) and urea (94.5 mg, 1.57 mmol); after purification (H₂O, 5-50% MeOH gradient) was obtained 75.2 mg 1-((4-bromo-1-methyl-1H-pyrazol-3-yl)methyl)dihydropyrimidine-2,4(1H,3H)-dione as beige solid. [M+H]⁺=287.

### ILB-87: 2-((2,4-dioxotetrahydropyrimidin-1(2H)-yl)methyl)thiazole-4-carboxylic acid

From 2-(aminomethyl)thiazole-4-carboxylic acid hydrochloride (76.3 mg, 0.39 mmol), acrylic acid (56.5 mg, 0.78 mmol), urea (70.6 mg, 1.18 mmol) and TEA (158.6 mg, 1.57 mmol); after purification (H₂O, 0-25% ACN gradient) was obtained 2.6 mg 2-((2,4-dioxotetrahydropyrimidin-1(2H)-yl)methyl)thiazole-4-carboxylic acid as yellow solid. [M+H]⁺=256.

### ILB-88: 1-((4-bromo-1H-pyrazol-5-yl)methyl)dihydropyrimidine-2,4(1H,3H)-dione

From (4-bromo-1H-pyrazol-5-yl)methanamine hydrobromide (94.1 mg, 0.37 mmol), acrylic acid (29.0 mg, 0.40 mmol), urea (66.0 mg, 1.10 mmol) and TEA (40.8 mg, 0.40 mmol); after purification (H₂O, 10-60% MeOH gradient) was obtained 2.8 mg 1-((4-bromo-1H-pyrazol-5-yl)methyl)dihydropyrimidine-2,4(1H,3H)-dione as white solid. [M+H]⁺=287.

### ILB-89: 1-((3-bromopyridin-4-yl)methyl)dihydropyrimidine-2,4(1H,3H)-dione

From (3-bromopyridin-4-yl)methanamine dihydrochloride (137.8 mg, 0.53 mmol), acrylic acid (76.4 mg, 1.06 mmol), urea (95.5 mg, 1.59 mmol) and TEA (118 mg, 1.17 mmol); after purification (H₂O, 10-60% MeOH gradient) was obtained 55.7 mg 1-((3-bromopyridin-4-yl)methyl)dihydropyrimidine-2,4(1H,3H)-dione as yellow solid. [M+H]⁺=284/286.

### ILB-90: 1-((5-bromopyrimidin-2-yl)methyl)dihydropyrimidine-2,4(1H,3H)-dione

From (5-bromopyrimidin-2-yl)methanamine hydrochloride (118.1 mg, 0.53 mmol), acrylic acid (75.8 mg, 1.05 mmol), urea (94.8 mg, 1.58 mmol) and TEA (58.6 mg, 0.58 mmol); after purification (H₂O, 5-55% MeOH gradient) was obtained 8.7 mg 1-((5-bromopyrimidin-2-yl)methyl)dihydropyrimidine-2,4(1H,3H)-dione as brown solid. [M+H]⁺=285/287.

### ILB-91: 1-((4-chloropyridin-2-yl)methyl)dihydropyrimidine-2,4(1H,3H)-dione

From (4-chloropyridin-2-yl)methanamine (89.2 mg, 0.63 mmol), acrylic acid (90.2 mg, 1.2 5 mmol) and urea (112.8 mg, 1.88 mmol); after purification (H₂O, 10-60% MeOH gradient) was obtained 2.4 mg 1-((4-chloropyridin-2-yl)methyl)dihydropyrimidine-2,4(1H,3H)-dione as yellow solid. [M+H]⁺=240.

### ILB-92: 1-((4-bromopyridin-2-yl)methyl)dihydropyrimidine-2,4(1H,3H)-dione

From (4-bromopyridin-2-yl)methanamine (98.7 mg, 0.53 mmol), acrylic acid (76.1 mg, 1.04 mmol) and urea (95.1 mg, 1.58 mmol); after purification (H₂O, 10-60% MeOH gradient) was obtained 2.9 mg 1-((4-bromopyridin-2-yl)methyl)dihydropyrimidine-2,4(1H,3H)-dione as yellow solid. [M+H]⁺=284/286.

### ILB-93: 1-((2-amino-5-bromopyridin-3-yl)methyl)dihydropyrimidine-2,4(1H,3H)-dione

From 3-(aminomethyl)-5-bromopyridin-2-amine (76.5 mg, 0.33 mmol), acrylic acid (26.5 mg, 0.37 mmol) and urea (60.2 mg, 1.0 mmol); after purification (H₂O, 10-60% MeOH gradient) was obtained 9.1 mg 1-((2-amino-5-bromopyridin-3-yl)methyl)dihydropyrimidine-2,4(1H,3H)-dione as yellow solid. [M+H]⁺=299/301.

### ILB-94: Benzyl-4-(3-((2,4-dioxotetrahydropyrimidin-1(2H)-yl)methyl)-2-oxopyridin-1(2H)-yl)piperidine-1-carboxylate

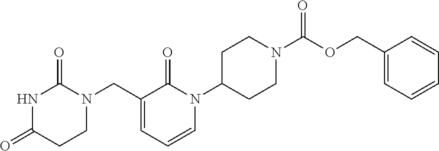 [SMILES: O=C1CCN(Cc2cccn(C3CCN(C(=O)OCc4ccccc4)CC3)c2=O)C(=O)N1]

### Step 1: Methyl 1-(1-((benzyloxy)carbonyl)piperidin-4-yl)-2-oxo-1,2-dihydropyridine-3-carboxylate

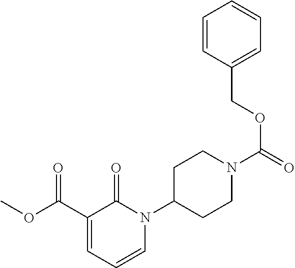 [SMILES: COC(=O)c1cccn(C2CCN(C(=O)OCc3ccccc3)CC2)c1=O]

This compound was prepared according to a procedure published in WO 2015/89143 A1.

### Step 2: Benzyl 4-(3-(hydroxymethyl)-2-oxopyridin-1(2H)-yl)piperidine-1-carboxylate

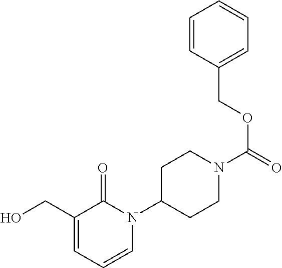 [SMILES: O=C(OCc1ccccc1)N1CCC(n2cccc(CO)c2=O)CC1]

To a solution of methyl 1-(1-((benzyloxy)carbonyl)piperidin-4-yl)-2-oxo-1,2-dihydropyridine-3-carboxylate (1 g, 2.295 mmol) in dry THF (23 mL) under Ar, was added at −4° C. (internal temperature) DIBAL-H, 1 M in toluene (5.05 mL, 5.05 mmol) portion wise in order not to exceed −1° C. Upon completion of the addition, the reaction was allowed to warm and was kept stirring between 0° C. and 5° C. for 120 min after which time additional DIBAL-H, 1 M in toluene (2 mL, 2 mmol) was added at 0° C. After 3 h at 0° C., the reaction was quenched with the portion wise addition of MeOH (1.3 mL), water (1.3 mL), 15% aq. NaOH (1.3 mL) and water (2.6 mL). The reaction was filtered on a pre-packed Celite® filter and the filtrate was concentrated in vacuo. The crude was purified by chromatography on a 40 g silica gel column eluting with MeOH in DCM (0 to 20%) over 21 min to afford the title compound as a yellow/orange solid (450 mg). Method LCMS9: Rt=0.75 min; [M+H]⁺=343.3.

### Step 3: Benzyl 4-(3-(bromomethyl)-2-oxopyridin-1(2H)-yl)piperidine-1-carboxylate

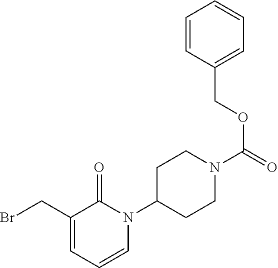 [SMILES: O=C(OCc1ccccc1)N1CCC(n2cccc(CBr)c2=O)CC1]

To a solution of benzyl 4-(3-(hydroxymethyl)-2-oxopyridin-1(2H)-yl)piperidine-1-carboxylate (450 mg, 1.235 mmol) in dry ACN (5 mL) at 0° C., under Ar, was added CBr₄ (574 mg, 1.730 mmol). PPh₃ (421 mg, 1.606 mmol) dissolved in dry ACN (6.5 mL) was added dropwise over 10 min. Upon completion of the addition, the reaction was further stirred at 0° C. for 2 h. The reaction was concentrated under reduced pressure to leave an oil which was purified by chromatography on a 24 g silica gel column eluting with EtOAc in cyclohexane (0 to 100%) over 23.5 min to afford the title compound as a white foam (330 mg). ¹H NMR (400 MHz, Chloroform-d) δ 7.48 (dd, J=6.8, 2.0 Hz, 1H), 7.41-7.31 (m, 5H), 7.30-7.22 (m, 1H), 6.23 (t, J=6.9 Hz, 1H), 5.26-5.07 (m, 3H), 4.47 (s, 2H), 4.45-4.27 (m, 2H), 3.13-2.87 (m, 2H), 1.99-1.87 (m, 2H), 1.78-1.59 (m, 2H).

### Step 4: Benzyl 4-(3-((2,4-dioxotetrahydropyrimidin-1(2H)-yl)methyl)-2-oxopyridin-1(2H)-yl)piperidine-1-carboxylate

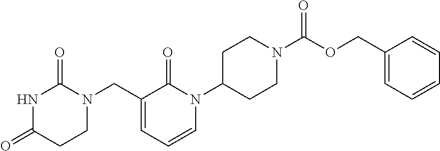 [SMILES: O=C1CCN(Cc2cccn(C3CCN(C(=O)OCc4ccccc4)CC3)c2=O)C(=O)N1]

To a solution of intermediate GG (3-((2-(trimethylsilyl)ethoxy)methyl)dihydropyrimidine-2,4(1H,3H)-dione (180 mg, 0.737 mmol) in dry DMF (5 mL) at RT, under Ar, was added NaH, 60% dispersion in mineral oil (58.9 mg, 1.473 mmol). After 5 min, benzyl 4-(3-(bromomethyl)-2-oxopyridin-1(2H)-yl)piperidine-1-carboxylate (320 mg, 0.790 mmol) in dry DMF (3 mL) was added dropwise to give a clear yellow solution. The reaction was stirred at RT for 50 min. At RT, the RM was poured portionwise in a sat. aq. NH₄Cl solution. EtOAc and water were added. The 2 layers were separated. The aqueous layer was extracted once with EtOAc. The combined organic layers were washed once with aq. 0.1M LiBr sol., once with a 1:1 mixture of a sat. aq. NaCl sol. and water, once with a sat. aq. NaCl sol., dried over MgSO₄, filtered, and evaporated to give a mixture of benzyl 4-(3-((2,4-dioxo-3-((2-(trimethylsilyl)ethoxy)methyl)tetrahydropyrimidin-1(2H)-yl)methyl)-2-oxopyridin-1(2H)-yl)piperidine-1-carboxylate and benzyl 4-(3-((3-(hydroxymethyl)-2,4-dioxotetrahydropyrimidin-1(2H)-yl)methyl)-2-oxopyridin-1(2H)-yl)piperidine-1-carboxylate. Chromatography on a 12 g silica gel column eluting with EtOAc in cyclohexane (20% to 100%) then MeOH in DCM (0% to 20%) over 26.4 min provided the pure benzyl 4-(3-((2,4-dioxo-3-((2-(trimethylsilyl)ethoxy)methyl)tetrahydropyrimidin-1(2H)-yl)methyl)-2-oxopyridin-1(2H)-yl)piperidine-1-carboxylate (91 mg). Method LCMS9: Rt=1.24 min; [M+H]⁺=569.5.

The remaining mixture (140 mg) was dissolved in DCM (2 mL) and TFA (1 mL) and stirred at RT for 15 min. The RM was evaporated to dryness, taken up in THF (1 mL) and a 5% NH₄OH sol. (1 mL) and stirred for 1 h. The RM was adsorbed on Isolute® HM-N and purified by reverse phase chromatography on a 15.5 g C18 column eluting with ACN (2-100%) in an aq. solution of TFA (0.1%) over 14.2 min. The obtained product was dissolved in MeOH (1.4 mL) and further purified by SFC using method XU (250×3 0 Reprospher PEI 100 A 5 μm, MeOH/CO2 14-22% in 9.3 min, total: 13 min) to afford the title compound as a white solid (3.5 mg). Method LCMS9: Rt=0.69 min; [M+H]⁺=439.4. ¹H NMR (400 MHz, DMSO-d₆) δ 10.15 (s, 1H), 7.70 (d, J=6.7 Hz, 1H), 7.41-7.37 (m, 4H), 7.36-7.29 (m, 1H), 7.26 (d, J=6.8 Hz, 1H), 6.25 (t, J=6.9 Hz, 1H), 5.10 (s, 2H), 5.02-4.84 (m, 1H), 4.27 (s, 2H), 4.17 (d, J=13.8 Hz, 2H), 3.42 (t, J=6.8 Hz, 2H), 3.12-2.85 (m, 2H), 2.57 (t, J=6.9 Hz, 2H), 1.82-1.71 (m, 4H).

### ILB-95: 2-(3-((2,4-Dioxotetrahydropyrimidin-1(2H)-yl)methyl)-2-oxopyridin-1(2H)-yl)acetic acid

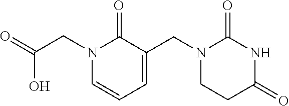 [SMILES: O=C(O)Cn1cccc(CN2CCC(=O)NC2=O)c1=O]

To a stirred solution of 2-(3-((2,4-dioxotetrahydropyrimidin-1(2H)-yl)methyl)-2-oxopyridin-1(2H)-yl)acetaldehyde (ILB-2, 60 mg, 0.228 mmol) in 3 mL of acetone at RT under argon was added KMnO₄ (72.0 mg, 0.456 mmol). The resulting purple RM was stirred at RT for 2 h. The RM was diluted with a 0.1% aq. solution of TFA and ACN, adsorbed on Isolute® and purified by reverse phase chromatography on a 5.5 g Redisep® C18 Gold column eluting with ACN (from 1 to 100%) in a 0.1% aq. solution of TFA to afford the title compound as an oil (15 mg). Method LCMS12: Rt=0.14 min; [M+H]⁺=280.1.

### Example 6: Bifunctional Degrader Synthesis

### Compound 01: N-(3-(6-(4-((4-(3-(2,4-dioxotetrahydropyrimidin-1(2H)-yl)-4-methoxybenzoyl)-1-oxa-4,9-diazaspiro[5.5]undecan-9-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

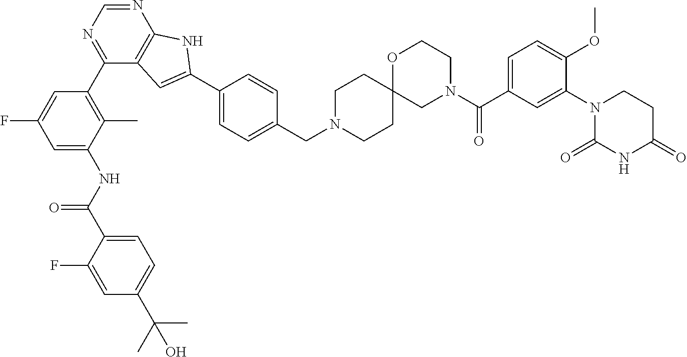 [SMILES: COc1ccc(C(=O)N2CCOC3(CCN(Cc4ccc(-c5cc6c(-c7cc(F)cc(NC(=O)c8ccc(C(C)(C)O)cc8F)c7C)ncnc6[nH]5)cc4)CC3)C2)cc1N1CCC(=O)NC1=O]

A mixture of 1-(2-methoxy-5-(1-oxa-4,9-diazaspiro[5.5]undecane-4-carbonyl)phenyl)dihydropyrimidine-2,4(1H,3H)-dione (ILB-57, 380 mg, 0.45 mmol) and K₂CO₃ (124 mg, 0.9 mmol) in DMSO (4 mL) was stirred at RT for 30 min. Then, 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 236 mg, 0.5 mmol) and ZnCl₂ 1 M in THF (0.5 mL, 0.5 mmol) were added. The resulting mixture was stirred at RT for 1 h. Then, NaBH₃CN (126 mg, 2 mmol) and MeOH (4 mL) were added. The mixture was stirred at RT for 16 h, then purified by reverse phase chromatography using Method PB on a Xtimate® C18 column eluting with ACN in an aq. solution of NH₄HCO₃ (10 mM) to afford the title compound as a yellow solid (120 mg). Method XI: Rt=0.96 min; [M+H]⁺=913. ¹H NMR (400 MHz, DMSO-d₆) δ 12.78 (s, 1H) 10.35 (s, 1H) 9.95 (s, 1H) 8.85 (s, 1H) 7.94 (s, 2H) 7.73 (t, J=8.0 Hz, 1H) 7.66 (d, J=9.6 Hz, 1H) 7.44-7.35 (m, 5H) 7.24 (d, J=8.9 Hz, 1H) 7.16 (d, J=8.9 Hz, 1H) 6.84 (s, 1H) 5.31 (s, 1H) 3.84 (s, 3H) 3.60-3.48 (m, 9H) 3.33-3.30 (m, 2H) 2.67 (s, 2H) 2.48-2.36 (m, 6H) 2.18 (s, 3H) 1.74 (s, 2H) 1.45 (s, 6H).

### Compound 02: N-(3-(6-(4-((9-(3-(2,4-dioxotetrahydropyrimidin-1(2H)-yl)-4-methoxybenzoyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

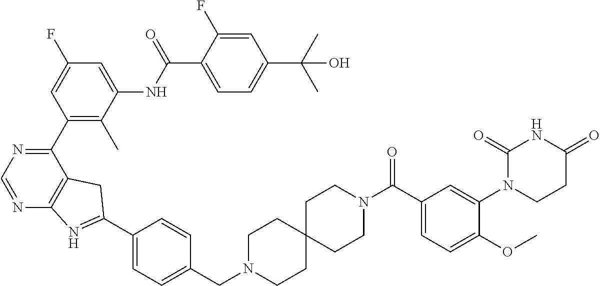 [SMILES: COc1ccc(C(=O)N2CCC3(CCN(Cc4ccc(C5=Nc6ncnc(-c7cc(F)cc(NC(=O)c8ccc(C(C)(C)O)cc8F)c7C)c6C5)cc4)CC3)CC2)cc1N1CCC(=O)NC1=O]

This compound was prepared as described in PCT/IB2019/052346 compound 9.

### Compound 03: N-(3-(6-(4-((4-(4-(4-(3-(2,4-dioxotetrahydropyrimidin-1(2H)-yl)phenyl)but-3-yn-1-yl)piperazine-1-carbonyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

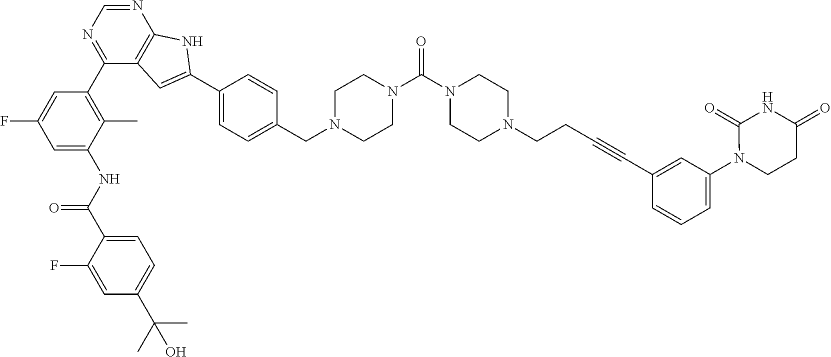 [SMILES: Cc1c(NC(=O)c2ccc(C(C)(C)O)cc2F)cc(F)cc1-c1ncnc2[nH]c(-c3ccc(CN4CCN(C(=O)N5CCN(CCC#Cc6cccc(N7CCC(=O)NC7=O)c6)CC5)CC4)cc3)cc12]

To a solution of 1-(3-(4-(4-(piperazine-1-carbonyl)piperazin-1-yl)but-1-yn-1-yl)phenyl)dihydropyrimidine-2,4(1H,3H)-dione (300 mg, 0.49 mmol) in DMSO (5 mL) was added K₂CO₃ (204 mg, 1.48 mmol). The solution was stirred for 30 min then 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 216 mg, 0.41 mmol) and ZnCl₂ (0.54 mL, 1 N in THF) were added. After 30 min, NaBH₃CN (46 mg, 0.74 mmol) was added. The reaction mixture was stirred at RT for 16 h. The solution was purified by reverse phase chromatography on a 120 g C18 Biotage® column eluting with ACN (0-60%) in an aq. solution of NH₄HCO₃ (0.1%) over 1 h to afford the title compound as a white solid (114 mg). Method G: Rt=1.77 min; [M+H]⁺=949. ¹H NMR (400 MHz, DMSO-d₆) δ 12.86-12.68 (m, 1H), 10.39 (s, 1H), 9.94 (d, J=2.6 Hz, 1H), 8.85 (s, 1H), 7.95 (d, J=8.2 Hz, 2H), 7.73 (t, J=7.9 Hz, 1H), 7.70-7.63 (m, 1H), 7.46-7.37 (m, 4H), 7.36-7.32 (m, 2H), 7.32-7.28 (m, 1H), 7.26-7.20 (m, 2H), 6.84 (d, J=1.7 Hz, 1H), 5.30 (s, 1H), 3.78 (t, J=6.6 Hz, 2H), 3.52 (s, 2H), 3.21-3.09 (m, 8H), 2.69 (t, J=6.6 Hz, 2H), 2.58 (s, 4H), 2.46-2.39 (m, 4H), 2.39-2.32 (m, 4H), 2.18 (s, 3H), 1.45 (s, 6H).

### Compound 04: 4-(dimethylamino)-3-((7-(3-(4-((1-(3-(2,4-dioxotetrahydropyrimidin-1(2H)-yl)-4-methoxybenzoyl)piperidin-4-yl)oxy)piperidin-1-yl)propoxy)quinazolin-4-yl)amino)-N-methylbenzenesulfonamide

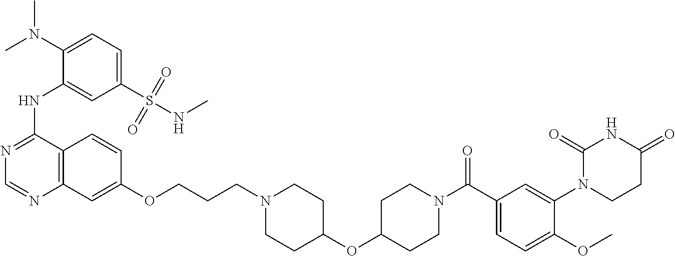 [SMILES: CNS(=O)(=O)c1ccc(N(C)C)c(Nc2ncnc3cc(OCCCN4CCC(OC5CCN(C(=O)c6ccc(OC)c(N7CCC(=O)NC7=O)c6)CC5)CC4)ccc23)c1]

To a suspension of 3-((7-(3-chloropropoxy)quinazolin-4-yl)amino)-4-(dimethylamino)-N-methylbenzenesulfonamide (Intermediate CC, 21.74 mg, 0.048 mmol) and 1-(2-methoxy-5-(4-(piperidin-4-yloxy)piperidine-1-carbonyl)phenyl)dihydropyrimidine-2,4(1H,3H)-dione (ILB-59, 20.8 mg, 0.048 mmol) in DMA (483 μL) was added potassium iodide (16.04 mg, 0.097 mmol) and DIPEA (50.6 μL, 0.290 mmol). The resulting solution was stirred at 80° C. for 90 min, then at 60° C. for 18 h. The reaction mixture was diluted with ACN and DMSO and purified via preparative HPLC (XBridge 30×50 mm 10-30% MeCN/H2O (0.1% formic acid) to afford, after filtration of the fractions containing the pure target compound through PL-HCO₃ MP SPE cartridges and freeze drying, the title compound as a white powder (20 mg). Method XR: Rt=2.04 min; [M+H]⁺=844.6. ¹H NMR (400 MHz, DMSO-d₆) δ 10.33 (s, 1H), 9.43 (s, 1H), 8.43 (s, 1H), 8.36 (d, J=9.2 Hz, 1H), 7.91 (d, J=2.3 Hz, 1H), 7.54 (dd, J=8.8, 2.4 Hz, 1H), 7.43-7.31 (m, 2H), 7.31-7.12 (m, 5H), 4.19 (t, J=6.2 Hz, 2H), 3.84 (s, 3H), 3.69 (s, 2H), 3.59 (t, J=6.6 Hz, 2H), 3.50 (s, 1H), 3.26-3.16 (m, 3H), 2.82 (s, 2H), 2.76 (s, 6H), 2.73-2.63 (m, 3H), 2.57 (d, J=13.5 Hz, 2H), 2.42 (d, J=5.2 Hz, 3H), 2.33 (q, J=1.8 Hz, 1H), 1.97 (s, 2H), 1.82 (s, 4H), 1.43 (s, 4H).

### Compound 05: 4-(dimethylamino)-3-((7-(3-(9-(3-(2,4-dioxotetrahydropyrimidin-1(2H)-yl)-4-methoxybenzoyl)-3,9-diazaspiro[5.5]undecan-3-yl)propoxy)quinazolin-4-yl)amino)-N-methylbenzenesulfonamide

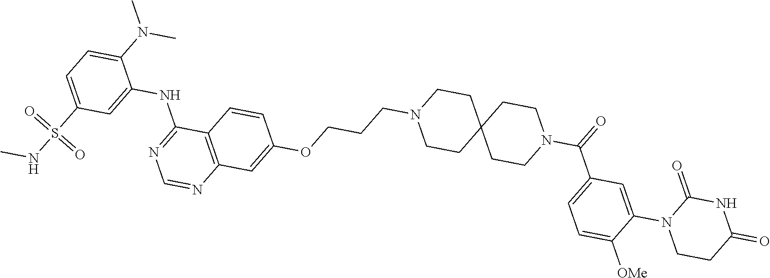 [SMILES: CNS(=O)(=O)c1ccc(N(C)C)c(Nc2ncnc3cc(OCCCN4CCC5(CC4)CCN(C(=O)c4ccc(OC)c(N6CCC(=O)NC6=O)c4)CC5)ccc23)c1]

To a suspension of 3-((7-(3-chloropropoxy)quinazolin-4-yl)amino)-4-(dimethylamino)-N-methylbenzenesulfonamide (Intermediate CC, 45.0 mg, 0.100 mmol) and 1-(2-methoxy-5-(3,9-diazaspiro[5.5]undecane-3-carbonyl)phenyl)dihydropyrimidine-2,4(1H,3H)-dione (ILB-56, 51.4 mg, 0.100 mmol) in DMA (1.0 mL) was added potassium iodide (16.60 mg, 0.100 mmol) and DIPEA (105 μL, 0.600 mmol). The resulting solution was stirred at 80° C. for 72 h. The RM was concentrated and the residue was purified via flash chromatography (0-40% MeOH/DCM) and then further via preparative HPLC (XBridge 30×50 mm 5-20% MeCN/H₂O (0.1% formic acid) to afford, after filtration of the fractions containing the pure target compound through PL-HCO₃ MP SPE cartridges and freeze drying, the title compound as a white powder (23 mg, 0.028 mmol). Method XR: Rt=2.07 min; [M+H]⁺=814.7. ¹H NMR (400 MHz, DMSO-d6) δ 10.33 (s, 1H), 9.42 (s, 1H), 8.43 (s, 1H), 8.35 (d, J=9.2 Hz, 1H), 7.91 (d, J=2.4 Hz, 1H), 7.54 (dd, J=8.6, 2.2 Hz, 1H), 7.37 (dd, J=8.5, 2.2 Hz, 1H), 7.32 (d, J=2.1 Hz, 1H), 7.27 (q, J=5.0 Hz, 1H), 7.25-7.13 (m, 4H), 4.17 (t, J=6.2 Hz, 2H), 3.84 (s, 3H), 3.60 (t, J=6.6 Hz, 2H), 3.45 (d, J=23.4 Hz, 4H), 2.76 (s, 6H), 2.68 (t, J=6.6 Hz, 2H), 2.46-2.41 (m, 5H), 2.41-2.31 (m, 4H), 2.00-1.88 (m, 2H), 1.51 (d, J=6.9 Hz, 4H), 1.42 (s, 4H).

### Compound 06: N-(2-chloro-6-methylphenyl)-2-((6-(4-(8-(9-(3-(2,4-dioxotetrahydropyrimidin-1(2H)-yl)-4-methoxybenzoyl)-3,9-diazaspiro[5.5]undecan-3-yl)-8-oxooctyl)piperazin-1-yl)-2-methylpyrimidin-4-yl)amino)thiazole-5-carboxamide (as TFA salt)

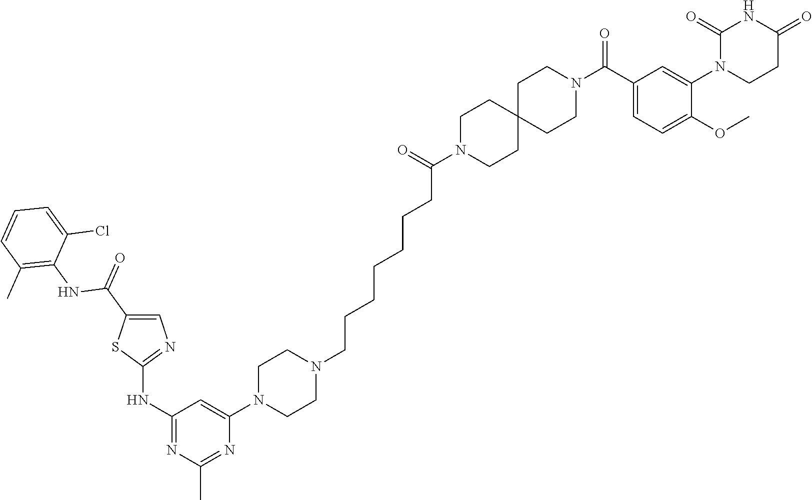 [SMILES: COc1ccc(C(=O)N2CCC3(CCN(C(=O)CCCCCCCN4CCN(c5cc(Nc6ncc(C(=O)Nc7c(C)cccc7Cl)s6)nc(C)n5)CC4)CC3)CC2)cc1N1CCC(=O)NC1=O]

### Step 1: N-(2-chloro-6-methylphenyl)-2-((2-methyl-6-(piperazin-1-yl)pyrimidin-4-yl)amino)thiazole-5-carboxamide

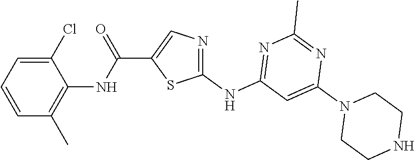 [SMILES: Cc1nc(Nc2ncc(C(=O)Nc3c(C)cccc3Cl)s2)cc(N2CCNCC2)n1]

To a suspension of 2-((6-chloro-2-methylpyrimidin-4-yl)amino)-N-(2-chloro-6-methylphenyl)thiazole-5-carboxamide (CAS No. [302964-08-5], 1 g, 2.54 mmol) in 1,4-Dioxane (30 mL) piperazine (CAS No. [110-85-0], 2.185 g, 25.4 mmol) and DIPEA (0.886 mL, 5.07 mmol) were added. The RM was heated at 100° C. under stirring for 2 h. Next it was allowed to reach RT and it was stirred for 5 days. The solvent was evaporated under reduced pressure to obtain a residue which was triturated sequentially in MeOH/H₂O/, Et₂O/H₂O, and Et₂O to obtain the title compound as a white solid (1.1 g). Method LCMS1: Rt=0.70 min. [M+H]⁺=444.1.

### Step 2: 8-(4-(6-((5-((2-chloro-6-methylphenyl)carbamoyl)thiazol-2-yl)amino)-2-methylpyrimidin-4-yl)piperazin-1-yl)octanoic acid

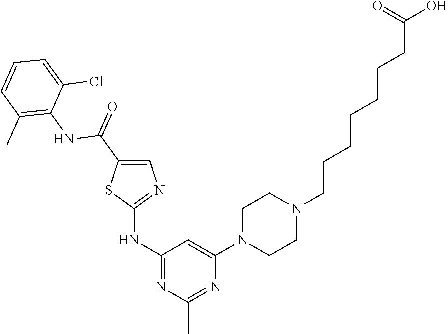 [SMILES: Cc1nc(Nc2ncc(C(=O)Nc3c(C)cccc3Cl)s2)cc(N2CCN(CCCCCCCC(=O)O)CC2)n1]

To a suspension of N-(2-chloro-6-methylphenyl)-2-((2-methyl-6-(piperazin-1-yl)pyrimidin-4-yl)amino)thiazole-5-carboxamide (CAS No. [910297-51-7] prepared according to J. Med. Chem. 50:5853-5857 (2007), 100 mg, 0.225 mmol) in 1,4-dioxane (1 mL) 8-bromooctanoic acid (CAS No. [17696-11-6], 50.0 mg, 0.225 mmol) and DIPEA (79 μL, 0.452 mmol) were added. The RM was heated to 100° C. and stirred for 3 days and subsequently stirred at RT for 2 days. The reaction mixture was diluted with DCM (10 mL) and washed with NaHCO₃ saturated aqueous solution (5 mL) and brine (5 mL). The combined organic layers were evaporated to dryness to obtain the solid crude material. The solid was dissolved in water and TFA was added until pH 1. This water solution was extracted with DCM:Isopropanol (7:3) and the organic phase was evaporated to obtain the crude material as a solid. Finally, the crude material was purified via preparative HPLC using the method XX (eluting with ACN in aq. TFA (0.1%) from 5% to 40%) to obtain, after freeze drying, the title compound as a TFA salt (150 mg). Method LCMS1: Rt=0.79 min; [M+H]⁺=586.3.

### Step 3: N-(2-chloro-6-methylphenyl)-2-((6-(4-(8-(9-(3-(2,4-dioxotetrahydropyrimidin-1(2H)-yl)-4-methoxybenzoyl)-3,9-diazaspiro[5.5]undecan-3-yl)-8-oxooctyl)piperazin-1-yl)-2-methylpyrimidin-4-yl)amino)thiazole-5-carboxamide

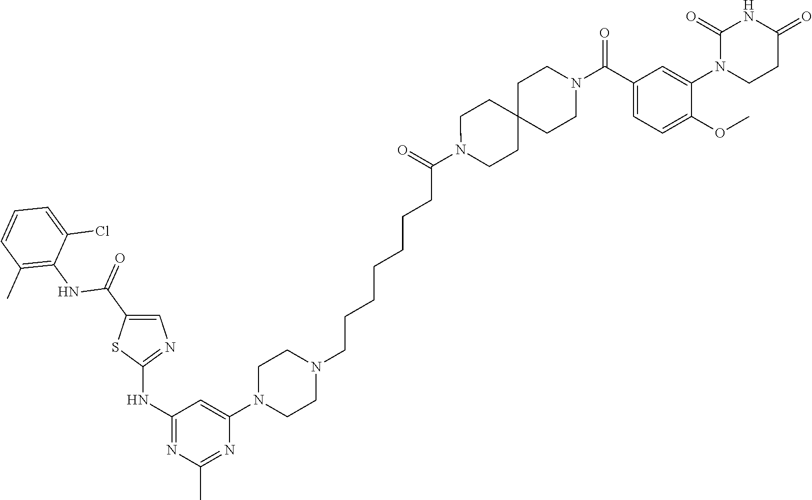 [SMILES: COc1ccc(C(=O)N2CCC3(CCN(C(=O)CCCCCCCN4CCN(c5cc(Nc6ncc(C(=O)Nc7c(C)cccc7Cl)s6)nc(C)n5)CC4)CC3)CC2)cc1N1CCC(=O)NC1=O]

TEA (0.023 mL, 0.166 mmol) was added to a mixture of 8-(4-(6-((5-((2-chloro-6-methylphenyl)carbamoyl)thiazol-2-yl)amino)-2-methylpyrimidin-4-yl)piperazin-1-yl)octanoic acid as TFA salt (27 mg, 0.017 mmol) and HBTU (12.59 mg, 0.033 mmol) in DMF (0.5 mL). The resulting RM was stirred at RT for 20 min. A solution of 1-(2-methoxy-5-(3,9-diazaspiro[5.5]undecane-3-carbonyl)phenyl)dihydropyrimidine-2,4(1H,3H)-dione as HCl salt (ILB-56, 12.81 mg, 0.025 mmol) in DMF (0.2 mL) was added. The resulting mixture was stirred at RT overnight. The reaction mixture was directly purified via preparative HPLC Method XX (eluting with ACN in aq. TFA (0.1%) from 5% to 50%) to afford, after freeze drying, the title compound as a white solid TFA salt (16 mg). Method LCMS1: Rt=0.84 min; [M+H]⁺=968.6 and [M−H]⁻=966.6. ¹H NMR (400 MHz, DMSO-d₆) δ 11.63 (s, 1H), 10.33 (s, 1H), 9.90 (s, 1H), 9.60 (s, 1H), 8.24 (s, 1H), 7.43-7.20 (m, 5H), 7.15 (d, J=8.6 Hz, 1H), 6.15 (s, 1H), 4.36 (d, J=12.7 Hz, 2H), 3.84 (s, 3H), 3.63-3.54 (m, 3H), 3.39 (s, 4H), 3.20 (dd, J=22.1, 9.5 Hz, 4H), 3.11 (s, 2H), 3.04 (d, J=9.5 Hz, 2H), 2.68 (t, J=6.4 Hz, 2H), 2.45 (s, 3H), 2.26 (d, J=17.5 Hz, 5H), 1.65 (s, 2H), 1.43 (d, J=28.4 Hz, 10H), 1.30 (s, 8H).

### Compound 07: N-(2-chloro-6-methylphenyl)-2-((6-(4-(2-(9-(3-(2,4-dioxotetrahydropyrimidin-1(2H)-yl)-4-methoxybenzoyl)-3,9-diazaspiro[5.5]undecan-3-yl)-2-oxoethyl)piperazin-1-yl)-2-methylpyrimidin-4-yl)amino)thiazole-5-carboxamide

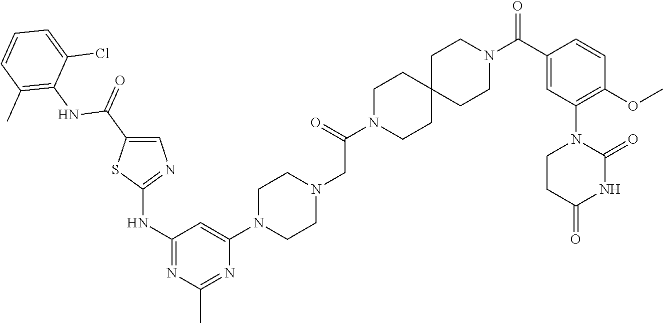 [SMILES: COc1ccc(C(=O)N2CCC3(CCN(C(=O)CN4CCN(c5cc(Nc6ncc(C(=O)Nc7c(C)cccc7Cl)s6)nc(C)n5)CC4)CC3)CC2)cc1N1CCC(=O)NC1=O]

### Step 1: tert-Butyl 2-(4-(6-((5-((2-chloro-6-methylphenyl)carbamoyl)thiazol-2-yl)amino)-2-methylpyrimidin-4-yl)piperazin-1-yl)acetate

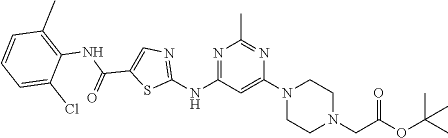 [SMILES: Cc1nc(Nc2ncc(C(=O)Nc3c(C)cccc3Cl)s2)cc(N2CCN(CC(=O)OC(C)(C)C)CC2)n1]

DIPEA (2 mL, 11.45 mmol) was added to a mixture of 2-((6-chloro-2-methylpyrimidin-4-yl)amino)-N-(2-chloro-6-methylphenyl)thiazole-5-carboxamide (CAS No. [302964-08-5], 500 mg, 1.268 mmol) and tert-butyl 2-(piperazin-1-yl)acetate (CAS No. [827614-56-2], 2000 mg, 9.99 mmol) in DMF (18 mL). The RM was heated in a sealed vial at 110° C. overnight. The RM was cooled to RT and then, it was diluted in EtOAc (100 mL). The obtained solution was washed with H₂O (100 mL×2), brine (100 mL×2), dried with MgSO₄, and evaporated to dryness. The crude material was purified by flash chromatography on a CombiFlash RF200 equipped with a RediSep® Column (silica 40 g), eluting with a gradient from 0% to 10% MeOH in DCM to afford after evaporation under reduced pressure a solid material. This was triturated in EtOAc (40 mL) to afford the title compound as a white solid (532 mg). Method LCMS1: Rt=0.61 min; [M+H]=558.2. ¹H NMR (400 MHz, DMSO-d6) δ 11.47 (s, 1H), 9.87 (s, 1H), 8.21 (s, 1H), 7.40 (d, J=6.8 Hz, 1H), 7.27 (d, J=10.1 Hz, 2H), 6.05 (s, 1H), 3.52 (s, 4H), 3.17 (s, 2H), 2.58 (s, 4H), 2.41 (s, 3H), 2.24 (s, 3H), 1.42 (s, 9H).

### Step 2: 2-(4-(6-((5-((2-chloro-6-methylphenyl)carbamoyl)thiazol-2-yl)amino)-2-methylpyrimidin-4-yl)piperazin-1-yl)acetic acid

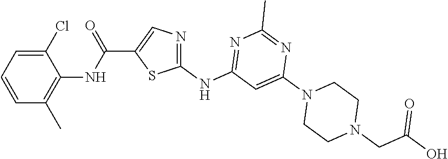 [SMILES: Cc1nc(Nc2ncc(C(=O)Nc3c(C)cccc3Cl)s2)cc(N2CCN(CC(=O)O)CC2)n1]

HCl in dioxane (4 M) (20 mL, 80 mmol) was added to a suspension of tert-butyl 2-(4-(6-((5-((2-chloro-6-methylphenyl)carbamoyl)thiazol-2-yl)amino)-2-methylpyrimidin-4-yl)piperazin-1-yl)acetate (532 mg, 0.953 mmol) in 50% dioxane/Water (10 mL). The RM was stirred at RT for 14 h. The solvent was removed under reduced pressure and the obtained solid was triturated in n-hexane to afford the title compound as a white solid HCl salt (580 mg). Method LCMS1: Rt=0.70 min; [M+H]⁺=502.2.

### Step 3: N-(2-chloro-6-methylphenyl)-2-((6-(4-(2-(9-(3-(2,4-dioxotetrahydropyrimidin-1(2H)-yl)-4-methoxybenzoyl)-3,9-diazaspiro[5.5]undecan-3-yl)-2-oxoethyl)piperazin-1-yl)-2-methylpyrimidin-4-yl)amino)thiazole-5-carboxamide

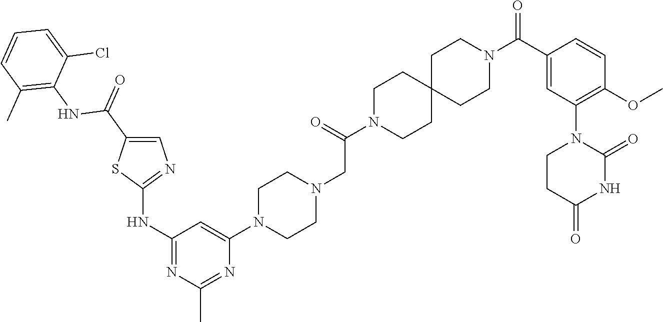 [SMILES: COc1ccc(C(=O)N2CCC3(CCN(C(=O)CN4CCN(c5cc(Nc6ncc(C(=O)Nc7c(C)cccc7Cl)s6)nc(C)n5)CC4)CC3)CC2)cc1N1CCC(=O)NC1=O]

TEA (0.045 mL, 0.324 mmol) was added to a mixture of 2-(4-(6-((5-((2-chloro-6-methylphenyl)carbamoyl)thiazol-2-yl)amino)-2-methylpyrimidin-4-yl)piperazin-1-yl)acetic acid (31 mg, 0.054 mmol) and HBTU (30.7 mg, 0.081 mmol) in DMF (1 mL). The RM was stirred at RT for 30 min. Next 1-(2-methoxy-5-(3,9-diazaspiro[5.5]undecane-3-carbonyl)phenyl)dihydropyrimidine-2,4(1H,3H)-dione (28.3 mg, 0.065 mmol) was added and the reaction was stirred at RT overnight. Finally, the RM was purified via preparative HPLC Method XX (eluting with ACN in aq. TFA (0.1%) from 5% to 70%) to afford, after freeze drying, the title compound as a white solid TFA salt (27 mg). Method LCMS1: Rt=0.80 min; [M+H]⁺=884.5. ¹H NMR (400 MHz, DMSO-d₆) δ 11.65 (s, 1H), 10.33 (s, 1H), 10.06 (s, 1H), 9.90 (s, 1H), 8.24 (s, 1H), 7.32 (m, 5H), 7.16 (d, J=8.6 Hz, 1H), 6.14 (s, 1H), 4.35 (s, 2H), 4.27 (s, 1H), 3.85 (s, 4H), 3.64-3.51 (m, 8H), 3.42 (s, 3H), 3.31 (s, 2H), 3.15 (d, J=16.7 Hz, 2H), 2.69 (d, J=6.6 Hz, 2H), 2.45 (s, 3H), 2.24 (s, 3H), 1.51 (d, J=20.8 Hz, 8H).

### Compound 08: N-(3-(6-(4-(((5-((5-(2,4-dioxotetrahydropyrimidin-1(2H)-yl)-2-methylbenzyl)amino)-5-oxopentyl)(methyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

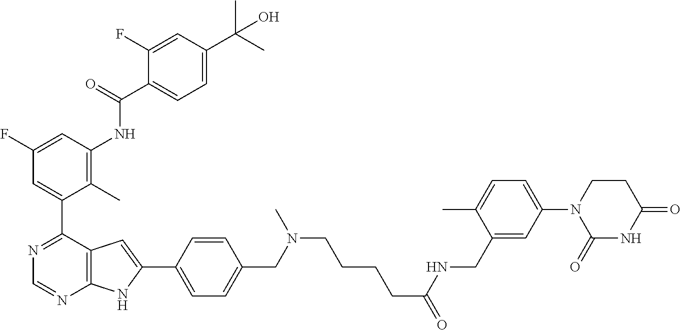 [SMILES: Cc1ccc(N2CCC(=O)NC2=O)cc1CNC(=O)CCCCN(C)Cc1ccc(-c2cc3c(-c4cc(F)cc(NC(=O)c5ccc(C(C)(C)O)cc5F)c4C)ncnc3[nH]2)cc1]

N-(5-(2,4-dioxotetrahydropyrimidin-1(2H)-yl)-2-methylbenzyl)-5-(methylamino)pentanamide (200 mg, 0.38 mmol) in DMSO (2 mL) was added to a mixture of K₂CO₃ (200 mg) and 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 145 mg, 0.42 mmol) in DMSO (3 mL), followed by the addition of ZnCl₂ 1 M (0.4 mL, 0.42 mmol). The RM was stirred at RT for 3 0 min. NaBH₃CN (48 mg, 0.76 mmol) was added to the RT. The RM was stirred at RT for an additional 30 min. MeOH (1 mL) was added to the mixture. The RM was stirred at RT for 16 h. The RM was concentrated and filtered to give the crude product as a brown liquid. The crude product was purified by reverse phase HPLC (5% to 95% ACN in H₂O, 0.1% NH₄HCO₃) using method PB to afford the title compound as a white solid (38 mg). Method G: Rt=1.77 min; [M+H]⁺=857. ¹H NMR (500 MHz, DMSO-d₆) δ 12.77 (s, 1H), 10.34 (s, 1H), 9.96 (s, 1H), 8.85 (s, 1H), 8.30 (t, J=5.7 Hz, 1H), 7.94 (d, J=7.8 Hz, 2H), 7.73 (t, J=8.0 Hz, 1H), 7.66-7.64 (m, 1H), 7.45-7.35 (m, 4H), 7.24 (dd, J=9.0, 2.8 Hz, 1H), 7.19 (d, J=7.6 Hz, 1H), 7.13-7.07 (m, 2H), 6.84 (s, 1H), 5.31 (s, 1H), 4.22 (d, J=5.9 Hz, 2H), 3.72-3.69 (m, 1H), 3.52-3.43 (m, 3H), 2.80-2.70 (m, 1H), 2.68-2.61 (m, 1H), 2.33 (t, J=7.2 Hz, 2H), 2.18 (s, 3H), 2.16-2.11 (m, 5H), 2.09 (s, 3H), 1.59-1.50 (m, 2H), 1.45 (s, 8H).

### Compound 09: N-(3-(6-(4-((4-(2-(2-(3-(2,4-dioxotetrahydropyrimidin-1(2H)-yl)phenoxy)acetamido)ethyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

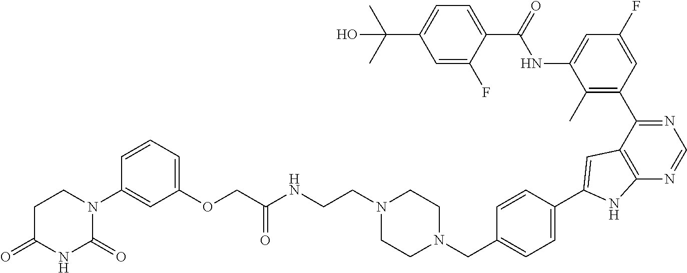 [SMILES: Cc1c(NC(=O)c2ccc(C(C)(C)O)cc2F)cc(F)cc1-c1ncnc2[nH]c(-c3ccc(CN4CCN(CCNC(=O)COc5cccc(N6CCC(=O)NC6=O)c5)CC4)cc3)cc12]

### Step 1: tert-Butyl 4-(2-(2-(3-(2,4-dioxotetrahydropyrimidin-1(2H)-yl)phenoxy)acetamido)ethyl)piperazine-1-carboxylate

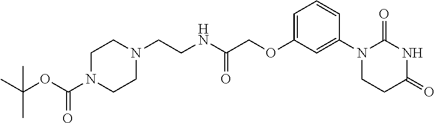 [SMILES: CC(C)(C)OC(=O)N1CCN(CCNC(=O)COc2cccc(N3CCC(=O)NC3=O)c2)CC1]

To a mixture of 2-(3-(2,4-dioxotetrahydropyrimidin-1(2H)-yl)phenoxy)acetic acid (ILB-16, 100 mg, 0.378 mmol) in DMF (2 mL) under argon was added NMM (0.208 mL, 1.892 mmol). The mixture was stirred at RT for 5 min. HATU (201 mg, 0.530 mmol) was added and the mixture was stirred at RT for 5 min. tert-Butyl 4-(2-aminoethyl)piperazine-1-carboxylate (174 mg, 0.757 mmol) was added and the RM was stirred at RT for 3 h. The RM was partially concentrated, adsorbed on Isolute® and purified by reverse phase chromatography on a Redisep® C18 column eluting with ACN in an aq. solution of TFA (0.1%) (from 2% to 100%), to afford the title compound as a TFA salt (185 mg). Method LCMS1: Rt=0.58 min; [M+H]⁺=476.3.

### Step 2: 2-(3-(2,4-dioxotetrahydropyrimidin-1(2H)-yl)phenoxy)-N-(2-(piperazin-1-yl)ethyl)acetamide

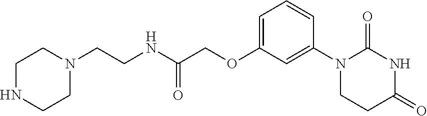 [SMILES: O=C(COc1cccc(N2CCC(=O)NC2=O)c1)NCCN1CCNCC1]

To a solution of tert-butyl 4-(2-(2-(3-(2,4-dioxotetrahydropyrimidin-1(2H)-yl)phenoxy)acetamido)ethyl)piperazine-1-carboxylate TFA salt (185 mg, 0.389 mmol) in DMF (2 mL) at 0° C. under argon was added a solution of HCl 4M in dioxane (5 mL, 20.0 mmol). The RM was stirred at RT for 45 min and concentrated. The resin was redissolved in a mixture of ACN and water and freeze dried to afford the HCl salt of the title compound as a white powder (145 mg). Method LCMS1: Rt=0.38 min; [M+H]⁺=376.3.

### Step 3: N-(3-(6-(4-((4-(2-(2-(3-(2,4-dioxotetrahydropyrimidin-1(2H)-yl)phenoxy)acetamido)ethyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

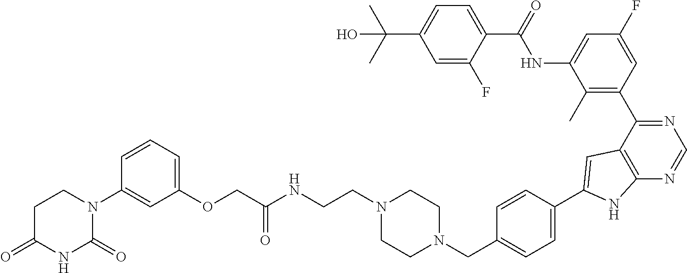 [SMILES: Cc1c(NC(=O)c2ccc(C(C)(C)O)cc2F)cc(F)cc1-c1ncnc2[nH]c(-c3ccc(CN4CCN(CCNC(=O)COc5cccc(N6CCC(=O)NC6=O)c5)CC4)cc3)cc12]

To a mixture of 2-(3-(2,4-dioxotetrahydropyrimidin-1(2H)-yl)phenoxy)-N-(2-(piperazin-1-yl)ethyl)acetamide HCl salt (120 mg, 0.291 mmol), 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 as described in PCT/IB2019/052346 184 mg, 0.350 mmol) were added TEA (0.081 mL, 0.583 mmol) and MeOH (0.5 mL) at RT under argon. The resulting mixture was stirred at RT for 5 min. A solution of ZnCl₂ 0.5 M in THF (0.699 mL, 0.350 mmol) was added and the RM was stirred at RT for 7.5 h. NaBH₃CN (27.5 mg, 0.437 mmol) was added and the RM was stirred at RT overnight. The RM was partially concentrated, adsorbed on Isolute® and purified by reverse phase chromatography on a Redisep® C18 column eluting with ACN in an aq. solution of formic acid (0.5%) (from 5% to 100%), to afford the title compound as a formic acid salt (105 mg). Method LCMS1: Rt=0.78 min; [M+H]⁺=886.6. ¹H NMR (600 MHz, DMSO-d₆) δ 13.66 (m, 1H), 10.40 (s, 1H), 10.08 (d, J=2.5 Hz, 1H), 9.15 (s, 1H), 8.39 (t, J=5.9 Hz, 1H), 8.17 (d, J=8.1 Hz, 2H), 7.84-7.77 (m, 1H), 7.74 (m, 1H), 7.64 (d, J=8.0 Hz, 2H), 7.47-7.41 (m, 2H), 7.38 (m, 1H), 7.32 (m, 1H), 7.24 (s, 1H), 6.99 (m, 1H), 6.96 (m, 1H), 6.86 (m, 1H), 4.53 (s, 2H), 4.33 (s, 2H), 3.80-3.07 (m, 14H), 2.70 (t, J=6.6 Hz, 2H), 2.20 (s, 3H), 1.46 (s, 6H).

### Compound 10: N-(3-(6-(4-(2-(4-(2-(3-(2,4-dioxotetrahydropyrimidin-1(2H)-yl)-4-methylphenoxy)acetyl)piperazin-1-yl)ethoxy)phenyl)-7H-pyrrolo[2,3-d]pyrimidin-4-yl)-5-fluoro-2-methylphenyl)-2-fluoro-4-(2-hydroxypropan-2-yl)benzamide

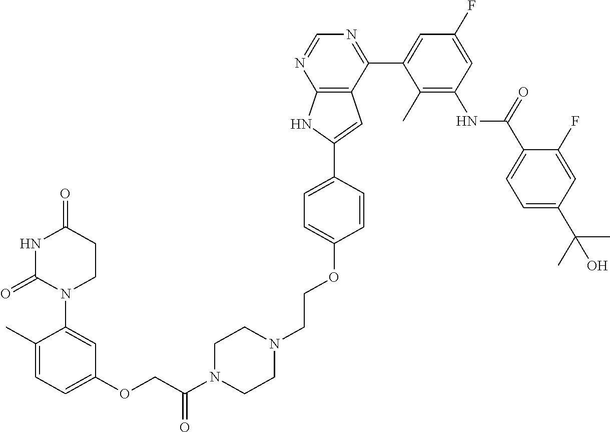 [SMILES: Cc1ccc(OCC(=O)N2CCN(CCOc3ccc(-c4cc5c(-c6cc(F)cc(NC(=O)c7ccc(C(C)(C)O)cc7F)c6C)ncnc5[nH]4)cc3)CC2)cc1N1CCC(=O)NC1=O]

### Step 1: tert-Butyl 4-(2-(4-(4-Chloro-7H-pyrrolo[2,3-d]pyrimidin-6-yl)phenoxy)ethyl)piperazine-1-carboxylate

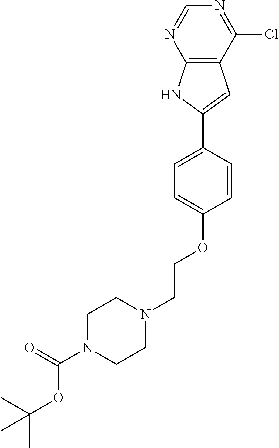 [SMILES: CC(C)(C)OC(=O)N1CCN(CCOc2ccc(-c3cc4c(Cl)ncnc4[nH]3)cc2)CC1]

A solution of 4-Chloro-6-iodo-7H-pyrrolo[2,3-d]pyrimidine (CAS No. [876343-10-1], 733 mg, 2.493 mmol), tert-butyl 4-(2-(4-(4,4,5,5-tetramethyl-1,3,2-dioxaborolan-2-yl)phenoxy)ethyl)piperazine-1-carboxylate (CAS No. [1310404-00-2], 1100 mg, 2.493 mmol) and cesium carbonate (2031 mg, 6.23 mmol) in 1,4-dioxane (7.5 mL) and water (7.5 mL) was degassed with argon. Pd(dppf)Cl₂ (200 mg, 0.245 mmol) was added and the RM was stirred at 100° C. for 1.5 h. The RM was partitioned between EtOAc and water and extracted. The organic phase was washed with brine, dried over MgSO₄, and concentrated. The crude was purified by chromatography on silica gel eluting with MeOH in DCM (from 0% to 12.5%) to afford the title compound as a beige solid (1183 mg). Method LCMS1: Rt=0.85 min; [M+H]⁺=458.3.

### Step 2: 2-Fluoro-N-(5-fluoro-2-methyl-3-(4,4,5,5-tetramethyl-1,3,2-dioxaborolan-2-yl)phenyl)-4-(2-hydroxypropan-2-yl)benzamide

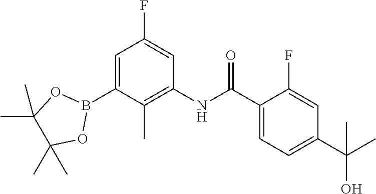 [SMILES: Cc1c(NC(=O)c2ccc(C(C)(C)O)cc2F)cc(F)cc1B1OC(C)(C)C(C)(C)O1]

A mixture of 2-fluoro-4-(2-hydroxypropan-2-yl)benzoic acid (6.35 g, 32.0 mmol), HATU (17.06 g, 44.9 mmol) and DIPEA (16.79 mL, 96 mmol) in DMF (100 mL) was stirred at RT for 30 min. Then, 5-fluoro-2-methyl-3-(4,4,5,5-tetramethyl-1,3,2-dioxaborolan-2-yl)aniline (can be prepared according to the procedure described in published patent application WO2013/008095 A1, page 37, intermediate 5) (8.47 g, 32.0 mmol) was added and the RM was stirred at 50° C. overnight. The RM was diluted with EtOAc and the organic phase was washed with a sat. aq. solution of NaHCO₃ and brine. The combined aqueous phases were extracted again with EtOAc and the combined organic phases were dried over Na₂SO₄, filtered, concentrated, and purified by chromatography on silica gel eluting with MeOH in DCM (from 0 to 10%). The resulting solid was triturated with Et₂O, filtered, the solids were washed with diisopropyl ether and dried to afford the title compound as a solid (9.28 g). Method A: Rt=1.28 min; [M+H]⁺=432.3. Note: An alternative preparation of intermediate 2 is described in WO2013/008095 A1, page 81, intermediate 35.

### Step 3: tert-butyl 4-(2-(4-(4-(5-fluoro-3-(2-fluoro-4-(2-hydroxypropan-2-yl)benzamido)-2-methylphenyl)-7H-pyrrolo[2,3-d]pyrimidin-6-yl)phenoxy)ethyl)piperazine-1-carboxylate

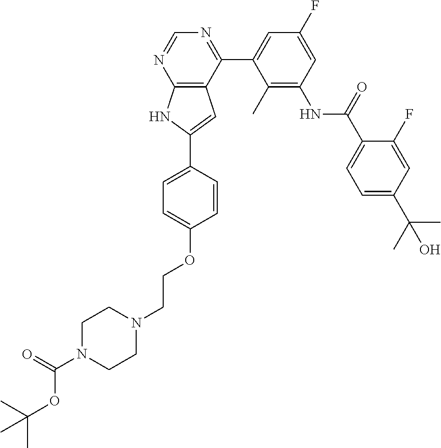 [SMILES: Cc1c(NC(=O)c2ccc(C(C)(C)O)cc2F)cc(F)cc1-c1ncnc2[nH]c(-c3ccc(OCCN4CCN(C(=O)OC(C)(C)C)CC4)cc3)cc12]

A solution of 2-fluoro-N-(5-fluoro-2-methyl-3-(4,4,5,5-tetramethyl-1,3,2-dioxaborolan-2-yl)phenyl)-4-(2-hydroxypropan-2-yl)benzamide, 535 mg, 1.178 mmol), tert-butyl 4-(2-(4-(4-chloro-7H-pyrrolo[2,3-d]pyrimidin-6-yl)phenoxy)ethyl)piperazine-1-carboxylate (535 mg, 0.946 mmol) and K₂CO₃ (330 mg, 2.366 mmol) in 1,4-dioxane (4 mL) and water (4 mL) was degassed with argon. Pd(dppf)Cl₂ (77 mg, 0.095 mmol) was added and the RM was stirred at 100° C. for 1.5 h. The RM was partitioned between EtOAc and water and extracted. The organic phase was washed with brine, dried over MgSO₄, and concentrated. The crude was purified by flash chromatography on silica gel eluting with MeOH in DCM (from 0% to 15%) to afford the title compound as an orange residue (561 mg). Method LCMS1: Rt=0.97 min; [M+H]⁺=727.4.

### Step 4: 2-fluoro-N-(5-fluoro-2-methyl-3-(6-(4-(2-(piperazin-1-yl)ethoxy)phenyl)-7H-pyrrolo[2,3-d]pyrimidin-4-yl)phenyl)-4-(2-hydroxypropan-2-yl)benzamide

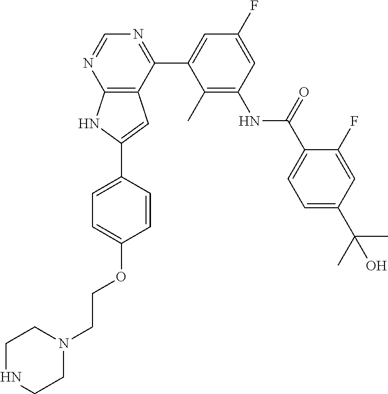 [SMILES: Cc1c(NC(=O)c2ccc(C(C)(C)O)cc2F)cc(F)cc1-c1ncnc2[nH]c(-c3ccc(OCCN4CCNCC4)cc3)cc12]

A solution of tert-butyl 4-(2-(4-(4-(5-fluoro-3-(2-fluoro-4-(2-hydroxypropan-2-yl)benzamido)-2-methylphenyl)-7H-pyrrolo[2,3-d]pyrimidin-6-yl)phenoxy)ethyl)piperazine-1-carboxylate (561 mg, 0.633 mmol) and TFA (1 mL, 12.98 mmol) in DCM (5 mL) was stirred at RT for 1.5 h. The RM was concentrated and purified by reverse phase chromatography on a Redisep® C18 column eluting with ACN in an aq. solution of TFA (0.1%) (from 2% to 100%), to afford the TFA salt of the title compound as yellow solid (477 mg). Method LCMS1: Rt=0.76 min; [M+H]⁺=627.5.

### Step 5: N-(3-(6-(4-(2-(4-(2-(3-(2,4-Dioxotetrahydropyrimidin-1(2H)-yl)-4-methylphenoxy)acetyl)piperazin-1-yl)ethoxy)phenyl)-7H-pyrrolo[2,3-d]pyrimidin-4-yl)-5-fluoro-2-methylphenyl)-2-fluoro-4-(2-hydroxypropan-2-yl)benzamide

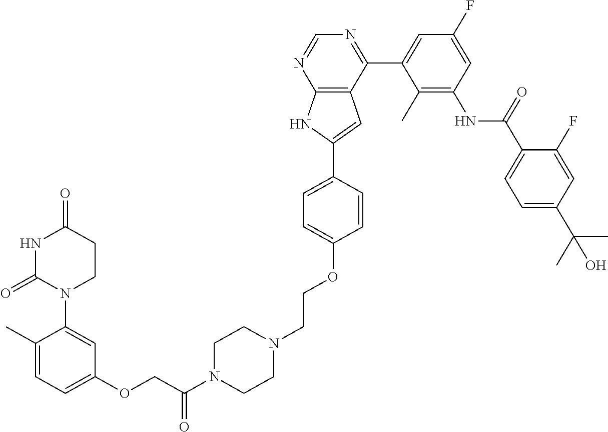 [SMILES: Cc1ccc(OCC(=O)N2CCN(CCOc3ccc(-c4cc5c(-c6cc(F)cc(NC(=O)c7ccc(C(C)(C)O)cc7F)c6C)ncnc5[nH]4)cc3)CC2)cc1N1CCC(=O)NC1=O]

To a mixture of 2-(3-(2,4-dioxotetrahydropyrimidin-1(2H)-yl)-4-methylphenoxy)acetic acid (ILB-20, 85 mg, 0.117 mmol) in DMF (2 mL) was added NMM (0.050 mL, 0.455 mmol). HATU (106 mg, 0.279 mmol) was added and the mixture was stirred at RT for 30 min. A solution of 2-fluoro-N-(5-fluoro-2-methyl-3-(6-(4-(2-(piperazin-1-yl)ethoxy)phenyl)-7H-pyrrolo[2,3-d]pyrimidin-4-yl)phenyl)-4-(2-hydroxypropan-2-yl)benzamide TFA salt (185 mg, 0.214 mmol) and NMM (0.050 mL, 0.455 mmol) was added and the RM was stirred at RT for 2.5 hr. The RM was purified by reverse phase chromatography on a Redisep® C18 column eluting with ACN in an aq. solution of TFA (0.1%) (from 2% to 100%). Fractions containing pure target compound were filtered through PL-HCO₃ MP SPE cartridges and freeze dried to afford crude material. The material was purified further by SFC Method XU on a Princeton PPU column (250×30 mm, 100 A, 5 μm) eluting with MeOH from 5% to 55%, to afford the title compound (36 mg). Method LCMS5: Rt=3.83 min; [M+H]⁺=887.4. ¹H NMR (400 MHz, DMSO-d₆) δ 9.92 (m, 2H), 8.74 (s, 1H), 7.91 (d, J=8.3 Hz, 2H), 7.72 (m, 1H), 7.61 (m, 1H), 7.48-7.34 (m, 2H), 7.25-7.17 (m, 1H), 7.14 (m, 1H), 7.01 (d, J=8.4 Hz, 2H), 6.87 (m, 1H), 6.79 (m, 1H), 6.65 (s, 1H), 5.29 (m, 1H), 4.75 (s, 2H), 4.14 (t, J=5.7 Hz, 2H), 3.74 (m, 1H), 3.51-3.40 (m, 7H), 2.82-2.58 (m, 6H), 2.16 (s, 3H), 2.07 (s, 3H), 1.44 (s, 6H).

### Compound 11: N-(3-(6-(4-((4-(2-(2-(3-(2,4-Dioxotetrahydropyrimidin-1(2H)-yl)phenoxy)acetamido)ethyl)piperidin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

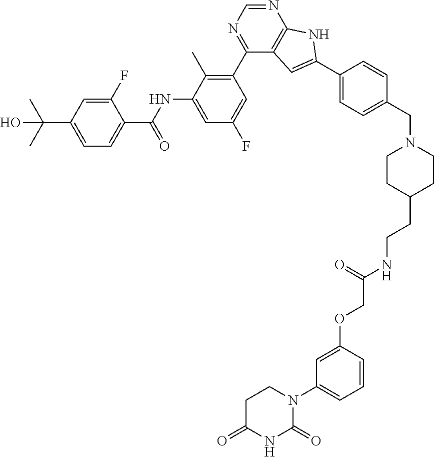 [SMILES: Cc1c(NC(=O)c2ccc(C(C)(C)O)cc2F)cc(F)cc1-c1ncnc2[nH]c(-c3ccc(CN4CCC(CCNC(=O)COc5cccc(N6CCC(=O)NC6=O)c5)CC4)cc3)cc12]

### Step 1: tert-Butyl 4-(2-(2-(3-(2,4-dioxotetrahydropyrimidin-1(2H)-yl)phenoxy)acetamido)ethyl)piperidine-1-carboxylate

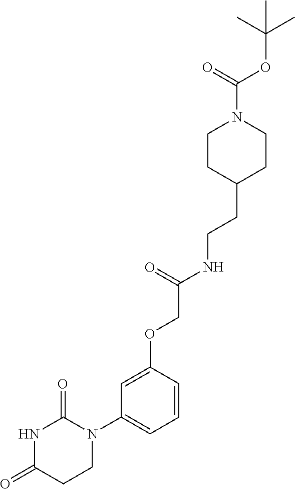 [SMILES: CC(C)(C)OC(=O)N1CCC(CCNC(=O)COc2cccc(N3CCC(=O)NC3=O)c2)CC1]

A mixture of 2-(3-(2,4-dioxotetrahydropyrimidin-1(2H)-yl)phenoxy)acetic acid (ILB-16, 100 mg, 0.375 mmol), tert-butyl 4-(2-aminoethyl)piperidine-1-carboxylate (90 mg, 0.394 mmol), NMM (0.050 mL, 0.455 mmol) and HATU (142 mg, 0.375 mmol) in DMF (2 mL) was stirred at RT for 3 h. The RM was purified by reverse phase chromatography on a Redisep® C18 column eluting with ACN in an aq. solution of TFA (0.1%) (from 2% to 100%), to afford the title compound as a white powder (150 mg). Method LCMS1: Rt=0.91 min; [M+H]⁺=475.3.

### Step 2: 2-(3-(2,4-Dioxotetrahydropyrimidin-1(2H)-yl)phenoxy)-N-(2-(piperidin-4-yl)ethyl)acetamide

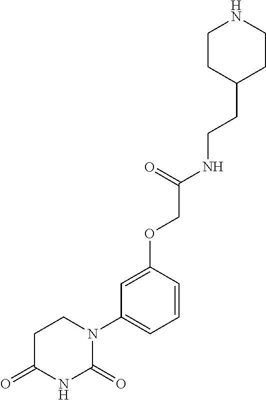 [SMILES: O=C(COc1cccc(N2CCC(=O)NC2=O)c1)NCCC1CCNCC1]

To a solution of tert-butyl 4-(2-(2-(3-(2,4-dioxotetrahydropyrimidin-1(2H)-yl)phenoxy)acetamido)ethyl)piperidine-1-carboxylate (143.5 mg, 0.302 mmol) in MeOH (2 mL) was added a solution of HCl 4 M in dioxane (1.5 mL, 6.0 mmol). The RM was stirred at RT for 1.5 h and concentrated to dryness, yielding the HCl salt of the title compound used in next step without further purification (131 mg). Method LCMS2: Rt=0.80 min; [M+H]⁺=375.3.

### Step 3: N-(3-(6-(4-((4-(2-(2-(3-(2,4-Dioxotetrahydropyrimidin-1(2H)-yl)phenoxy)acetamido)ethyl)piperidin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

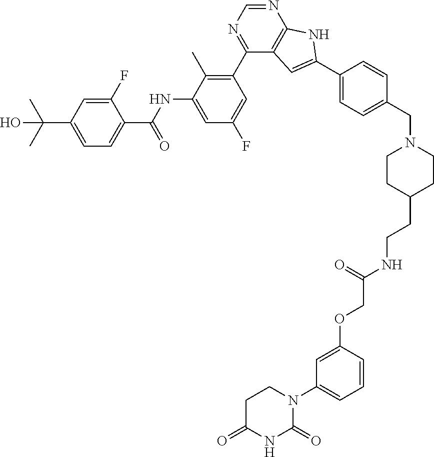 [SMILES: Cc1c(NC(=O)c2ccc(C(C)(C)O)cc2F)cc(F)cc1-c1ncnc2[nH]c(-c3ccc(CN4CCC(CCNC(=O)COc5cccc(N6CCC(=O)NC6=O)c5)CC4)cc3)cc12]

To a mixture of 2-(3-(2,4-dioxotetrahydropyrimidin-1(2H)-yl)phenoxy)-N-(2-(piperidin-4-yl)ethyl)acetamide HCl salt (130 mg, 0.302 mmol), and 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 as described in PCT/IB2019/052346 162 mg, 0.302 mmol) were added TEA (0.100 mL, 0.717 mmol) and MeOH (2 mL) at RT. A solution of ZnCl₂ 0.5 M in THF (0.700 mL, 0.350 mmol) was added and the RM was stirred at RT for 3 h. NaBH₃CN (22 mg, 0.350 mmol) was added and the RM was stirred at RT overnight. The RM was concentrated and purified by reverse phase chromatography on a Redisep® C18 column eluting with ACN in an aq. solution of TFA (0.1%) (from 2% to 100%). Fractions containing pure target compound were filtered through PL-HCO₃ MP SPE cartridges and freeze dried to afford crude material. The material was purified further by SFC (Method XU) on a Reprospher PEI column (250×30 mm, 100 Å, 5 μm) eluting with MeOH from 22% to 54%, yielding the title compound (118 mg). Method LCMS5: Rt=3.73 min; [M+H]⁺=885.4. ¹H NMR (400 MHz, DMSO-d₆) δ 12.75 (s, 1H), 10.36 (s, 1H), 9.93 (s, 1H), 8.84 (d, J=2.7 Hz, 1H), 8.04 (m, 1H), 7.93 (d, J=7.7 Hz, 2H), 7.72 (m, 1H), 7.64 (m, 1H), 7.39 (m, 4H), 7.32-7.18 (m, 2H), 6.94 (m, 2H), 6.82 (m, 2H), 5.28 (s, 1H), 4.52-4.36 (s, 2H), 3.75 (m, 2H), 3.49 (m, 2H), 3.15 (m, 2H), 2.79 (m, 2H), 2.68 (m, 2H), 2.16 (s, 3H), 1.91 (m, 2H), 1.61 (s, 2H), 1.51-1.30 (m, 9H), 1.15 (m, 2H).

### Compound 12: N-(3-(6-(4-(((3-(2-(3-(2,4-dioxotetrahydropyrimidin-1(2H)-yl)phenoxy)acetamido)propyl)(methyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

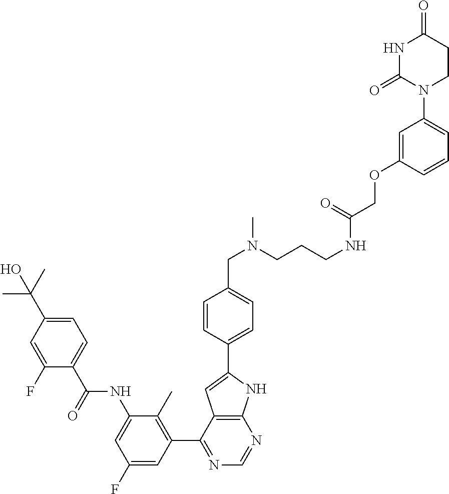 [SMILES: Cc1c(NC(=O)c2ccc(C(C)(C)O)cc2F)cc(F)cc1-c1ncnc2[nH]c(-c3ccc(CN(C)CCCNC(=O)COc4cccc(N5CCC(=O)NC5=O)c4)cc3)cc12]

### Step 1: tert-Butyl (3-((4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)(methyl)amino)propyl)carbamate

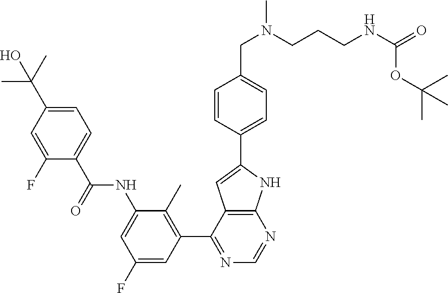 [SMILES: Cc1c(NC(=O)c2ccc(C(C)(C)O)cc2F)cc(F)cc1-c1ncnc2[nH]c(-c3ccc(CN(C)CCCNC(=O)OC(C)(C)C)cc3)cc12]

To a mixture of 3-(methylaminopropyl)carbamic acid tert-butyl ester (129 mg, 0.684 mmol), 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 300 mg, 0.570 mmol) in MeOH (5 mL) at RT was added a solution of ZnCl₂ 0.5 M in THF (1.4 mL, 0.700 mmol) and the RM was stirred at RT for 2 days. NaBH₃CN (40 mg, 0.637 mmol) was added and the RM was stirred at RT overnight. The RM was concentrated and purified by chromatography on silica gel eluting with MeOH in DCM (from 0% to 50%) yielding the title compound (400 mg). Method LCMS1: Rt=0.87 min; [M+H]⁺=699.4.

### Step 2: N-(3-(6-(4-(((3-aminopropyl)(methyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

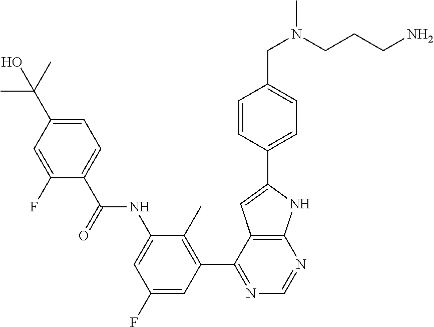 [SMILES: Cc1c(NC(=O)c2ccc(C(C)(C)O)cc2F)cc(F)cc1-c1ncnc2[nH]c(-c3ccc(CN(C)CCCN)cc3)cc12]

To a solution of tert-butyl (3-((4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)(methyl)amino)propyl)carbamate (400 mg, 0.561 mmol) in MeOH (2 mL) was added a solution of HCl 4 M in dioxane (2 mL, 8.0 mmol). The RM was stirred at RT for 1 h and concentrated, yielding the HCl salt of the title compound as a yellow solid (395 mg). Method LCMS1: Rt=0.67 min; [M+H]⁺=599.4.

### Step 3: N-(3-(6-(4-(((3-(2-(3-(2,4-dioxotetrahydropyrimidin-1(2H)-yl)phenoxy)acetamido)propyl)(methyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

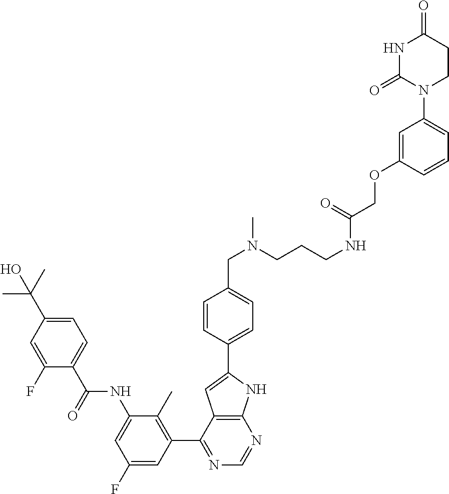 [SMILES: Cc1c(NC(=O)c2ccc(C(C)(C)O)cc2F)cc(F)cc1-c1ncnc2[nH]c(-c3ccc(CN(C)CCCNC(=O)COc4cccc(N5CCC(=O)NC5=O)c4)cc3)cc12]

To a mixture of 2-(3-(2,4-dioxotetrahydropyrimidin-1(2H)-yl)phenoxy)acetic acid (ILB-16, 42 mg, 0.153 mmol) in DMF (0.5 mL) was added NMM (0.020 mL, 0.182 mmol). HATU (60 mg, 0.158 mmol) was added and the mixture was stirred at RT for 30 min. A solution of N-(3-(6-(4-(((3-aminopropyl)(methyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide HCl salt (95 mg, 0.151 mmol) and NMM (0.020 mL, 0.182 mmol) in DMF (1 mL) was added and the RM was stirred at RT for 1.5 h. The RM was purified by reverse phase chromatography on a Redisep® C18 column eluting with ACN in an aq. solution of TFA (0.1%) (from 5% to 100%). Fractions containing pure target compound were filtered through PL-HCO₃ MP SPE cartridges and freeze dried to afford crude material. The material was purified further by SFC Method XU on a Princeton PPU column (250×30 mm, 100 Å, 5 μm) eluting with MeOH from 5% to 55%, yielding the title compound (24 mg). Method LCMS5: Rt=3.46 min; [M+H]⁺=845.4. ¹H NMR (400 MHz, DMSO-d6) δ 12.81 (m, 2H), 10.35 (s, 1H), 9.94 (s, 1H), 8.86 (s, 1H), 8.04 (m, 3H), 7.78-7.34 (m, 6H), 7.24 (m, 2H), 7.08-6.74 (m, 4H), 5.29 (s, 1H), 4.45 (s, 2H), 3.74 (m, 4H), 3.49 (m, 4H), 2.66 (m, 2H), 2.50 (s, 3H), 2.16 (s, 3H), 1.44 (s, 6H), 1.22 (m, 2H).

### Compound 13: N-(3-(6-(6-(3-(4-(2-(4-chloro-3-(2,4-dioxotetrahydropyrimidin-1(2H)-yl)phenoxy)acetyl)piperazin-1-yl)propoxy)pyridin-3-yl)-7H-pyrrolo[2,3-d]pyrimidin-4-yl)-5-fluoro-2-methylphenyl)-3-hydroxy-3-neopentylazetidine-1-carboxamide

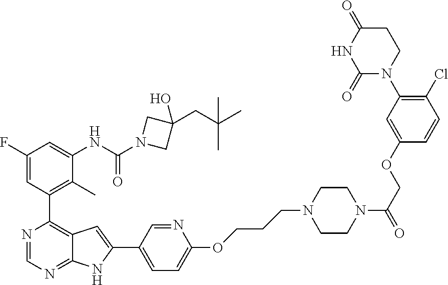 [SMILES: Cc1c(NC(=O)N2CC(O)(CC(C)(C)C)C2)cc(F)cc1-c1ncnc2[nH]c(-c3ccc(OCCCN4CCN(C(=O)COc5ccc(Cl)c(N6CCC(=O)NC6=O)c5)CC4)nc3)cc12]

### Step 1: tert-Butyl 3-hydroxy-3-neopentylazetidine-1-carboxylate

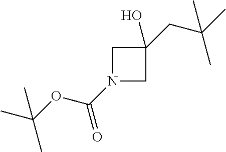 [SMILES: CC(C)(C)CC1(O)CN(C(=O)OC(C)(C)C)C1]

1-Boc-3-azetidinone (5 g, 29.2 mmol) was dissolved in a solution of lanthanum(III)chloride bis(lithium chloride) 0.6M in THF (48.7 mL, 29.2 mmol) and stirred for 1 h at RT. The mixture was cooled at 0° C. A solution of 2,2-dimethylpropylmagnesium chloride 1.0 M in Et₂O (30.7 mL, 30.7 mmol) was added dropwise. The RM was stirred at 0° C. for 1 h, quenched with aq. sat. NH₄Cl sol. (29 mL) and H₂O (29 mL), filtered over Hyflo® and rinsed with Et₂O. The aqueous layer of the filtrate was extracted twice with Et₂O. The organic phase was dried over Na₂SO₄ and evaporated to dryness, yielding the title compound as a beige solid (6.6 g). ¹H NMR (400 MHz, chloroform-d) δ 1.02 (s, 9H) 1.43 (s, 9H) 1.73 (s, 2H) 3.78 (d, J=9.35 Hz, 2H) 3.94 (d, J=9.35 Hz, 2H).

### Step 2: 3-Neopentylazetidin-3-ol

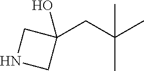

To a mixture of tert-butyl 3-hydroxy-3-neopentylazetidine-1-carboxylate (1.6 g, 6.58 mmol) in DCM (60 mL) was added TFA (5.07 mL, 65.8 mmol) at RT. The RM was stirred at RT for 4 h and concentrated to dryness to afford the TFA salt of the title compound as a brown oil (2.6 g). ¹H NMR (400 MHz, chloroform-d) δ 1.02 (s, 9H), 1.80 (s, 2H) 4.12 (m, 2H) 4.26 (m, 2H), 8.08 (m, 1H), 8.76 (m, 1H).

### Step 3: N-(5-Fluoro-2-methyl-3-(4,4,5,5-tetramethyl-1,3,2-dioxaborolan-2-yl)phenyl)-3-hydroxy-3-neopentylazetidine-1-carboxamide

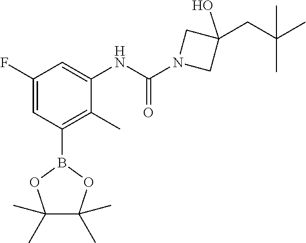 [SMILES: Cc1c(NC(=O)N2CC(O)(CC(C)(C)C)C2)cc(F)cc1B1OC(C)(C)C(C)(C)O1]

To a solution of 5-fluoro-2-methyl-3-(4,4,5,5-tetramethyl-1,3,2-dioxaborolan-2-yl)aniline (CAS [1418128-33-2], 3.6 g, 14.34 mmol) and DIPEA (10.02 mL, 57.3 mmol) in DCM (40 mL) was slowly added a solution of phosgene 20% in toluene (9.05 mL, 17.20 mmol) at 0° C. The resulting solution was stirred at 0° C. for 10 min, then transferred to a stirring solution of 3-neopentylazetidin-3-ol TFA salt (4.06 g, 15.77 mmol) in DCM (40.0 mL) at 0° C. The RM was stirred at 0° C. for 1 h. The RM was concentrated, then poured into water and extracted three times with EtOAc. The organic phase was washed with H₂O and brine, dried with MgSO₄, and concentrated. The crude product was triturated in DCM/MTBE (1:5), then filtered off and dried under HV yielding the title compound as white crystals (4 g). The filtrate was concentrated and crystallized again in TBME to afford the title compound as white crystals (1 g). Method LCMS1: Rt=1.26 min; [M+H]⁺=421.4.

### Step 4: 5-(4-Chloro-7-((2-(trimethylsilyl)ethoxy)methyl)-7H-pyrrolo[2,3-d]pyrimidin-6-yl)pyridin-2-ol

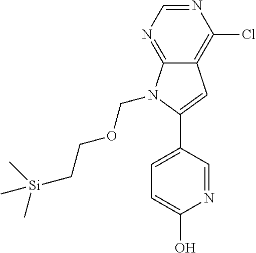 [SMILES: C[Si](C)(C)CCOCn1c(-c2ccc(O)nc2)cc2c(Cl)ncnc21]

To a solution of 4-chloro-6-iodo-7-((2-(trimethylsilyl)ethoxy)methyl)-7H-pyrrolo[2,3-d]pyrimidine (intermediate 15 in PCT/IB2019/052346 3 g, 7.32 mmol), 5-(4,4,5,5-tetramethyl-1,3,2-dioxaborolan-2-yl)pyridin-2-ol (1800 mg, 8.14 mmol) and a solution of tripotassium phosphate 2M in H₂O (8 mL, 216.00 mmol) in degassed 1,4-dioxane (30 mL) was added Pd(dppf)Cl₂ (540 mg, 0.738 mmol). The RM was stirred at 90° C. for 1 h. 5-(4,4,5,5-tetramethyl-1,3,2-dioxaborolan-2-yl)pyridin-2-ol (900 mg, 4.39 mmol) was added and the RM was stirred at 90° C. for 2 h. The RM was partitioned between EtOAc and water and extracted. The organic phase was washed twice with H₂O and once with brine, dried over Na₂SO₄, and evaporated. The crude was purified by chromatography on silica gel eluting with EtOAc in hexane (from 0% to 100%) yielding the title compound as a yellow residue (270 mg). Method LCMS1: Rt=1.09 min; [M+H]⁺=377.2.

### Step 5: tert-Butyl 4-(3-((5-(4-chloro-7-((2-(trimethylsilyl)ethoxy)methyl)-7H-pyrrolo[2,3-d]pyrimidin-6-yl)pyridin-2-yl)oxy)propyl)piperazine-1-carboxylate

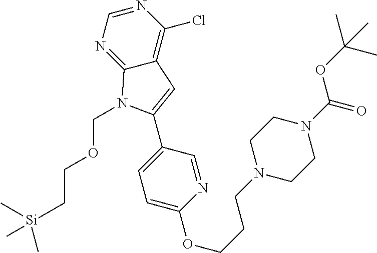 [SMILES: CC(C)(C)OC(=O)N1CCN(CCCOc2ccc(-c3cc4c(Cl)ncnc4n3COCC[Si](C)(C)C)cn2)CC1]

To a mixture of 5-(4-chloro-7-((2-(trimethylsilyl)ethoxy)methyl)-7H-pyrrolo[2,3-d]pyrimidin-6-yl)pyridin-2-ol (270 mg, 0.609 mmol), tert-butyl 4-(3-hydroxypropyl)piperazine-1-carboxylate (298 mg, 1.218 mmol), PPh₃ (399 mg, 1.522 mmol) and anhydrous THF (5 mL) was added dropwise DIAD (0.296 mL, 1.522 mmol) at 0° C. and the RM was stirred at 0° C. for 20 min. The RM was evaporated and purified by chromatography on silica gel eluting with EtOAc in hexane yielding the title compound as a yellow residue (218 mg). Method LCMS5: Rt=5.95 min; [M+H]⁺=603.4.

### Step 6: tert-Butyl 4-(3-((5-(4-(5-fluoro-3-(3-hydroxy-3-neopentylazetidine-1-carboxamido)-2-methylphenyl)-7-((2-(trimethylsilyl)ethoxy)methyl)-7H-pyrrolo[2,3-d]pyrimidin-6-yl)pyridin-2-yl)oxy)propyl)piperazine-1-carboxylate

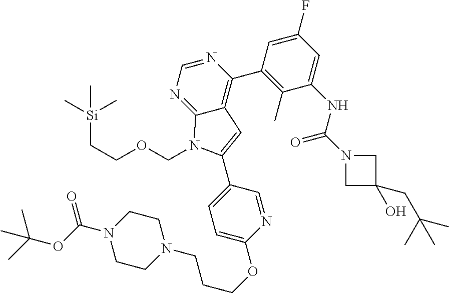 [SMILES: Cc1c(NC(=O)N2CC(O)(CC(C)(C)C)C2)cc(F)cc1-c1ncnc2c1cc(-c1ccc(OCCCN3CCN(C(=O)OC(C)(C)C)CC3)nc1)n2COCC[Si](C)(C)C]

To a solution of tert-butyl 4-(3-((5-(4-chloro-7-((2-(trimethylsilyl)ethoxy)methyl)-7H-pyrrolo[2,3-d]pyrimidin-6-yl)pyridin-2-yl)oxy)propyl)piperazine-1-carboxylate (215 mg, 0.356 mmol), N-(5-fluoro-2-methyl-3-(4,4,5,5-tetramethyl-1,3,2-dioxaborolan-2-yl)phenyl)-3-hydroxy-3-neopentylazetidine-1-carboxamide (225 mg, 0.535 mmol) and a solution of tripotassium phosphate 2 M in H₂O (0.446 mL, 0.891 mmol) in degassed 1,4-dioxane (2.5 mL) was added [1,1′-Bis(di-tert-butylphosphino)ferrocene]dichloropalladium (II) (23.23 mg, 0.036 mmol). The RM was stirred at RT for 5 min. The RM was partitioned between EtOAc and water and extracted. The organic phase was washed with H₂O and with brine, dried over Na₂SO₄, and evaporated. The crude was purified by chromatography on silica gel eluting with EtOAc/EtOH (95:5) in hexane yielding the title compound as a yellow residue (270 mg). Method LCMS5: Rt=6.27 min; [M+H]⁺=861.6.

### Step 7: N-(5-Fluoro-2-methyl-3-(6-(6-(3-(piperazin-1-yl)propoxy)pyridin-3-yl)-7H-pyrrolo[2,3-d]pyrimidin-4-yl)phenyl)-3-hydroxy-3-neopentylazetidine-1-carboxamide

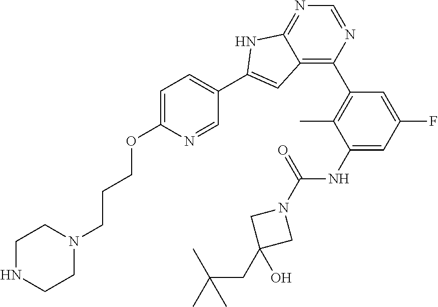 [SMILES: Cc1c(NC(=O)N2CC(O)(CC(C)(C)C)C2)cc(F)cc1-c1ncnc2[nH]c(-c3ccc(OCCCN4CCNCC4)nc3)cc12]

To a mixture of tert-butyl 4-(3-((5-(4-(5-fluoro-3-(3-hydroxy-3-neopentylazetidine-1-carboxamido)-2-methylphenyl)-7-((2-(trimethylsilyl)ethoxy)methyl)-7H-pyrrolo[2,3-d]pyrimidin-6-yl)pyridin-2-yl)oxy)propyl)piperazine-1-carboxylate (168 mg, 0.195 mmol) in DCM (2 mL) was added TFA (0.750 mL) at RT. The RM was stirred at RT for 3 h. TFA (0.300 mL) was added and the RM was stirred at RT overnight. The crude was diluted with MeOH (1 mL) and filtered through a PoraPak cartridge (20 mL, 2 g), yielding the title compound as a yellow residue (120 mg). Method LCMS1: Rt=0.73 min; [M+H]⁺=631.5.

### Step 8: N-(3-(6-(6-(3-(4-(2-(4-Chloro-3-(2,4-dioxotetrahydropyrimidin-1(2H)-yl)phenoxy)acetyl)piperazin-1-yl)propoxy)pyridin-3-yl)-7H-pyrrolo[2,3-d]pyrimidin-4-yl)-5-fluoro-2-methylphenyl)-3-hydroxy-3-neopentylazetidine-1-carboxamide

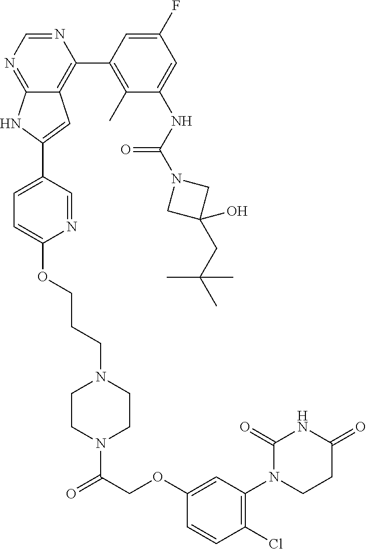 [SMILES: Cc1c(NC(=O)N2CC(O)(CC(C)(C)C)C2)cc(F)cc1-c1ncnc2[nH]c(-c3ccc(OCCCN4CCN(C(=O)COc5ccc(Cl)c(N6CCC(=O)NC6=O)c5)CC4)nc3)cc12]

To a mixture of 2-(4-chloro-3-(2,4-dioxotetrahydropyrimidin-1(2H)-yl)phenoxy)acetic acid (20.27 mg, 0.061 mmol), TEA (0.020, 0.141 mmol) and HATU (30.4 mg, 0.080 mmol) in DMF (0.5 mL). The mixture was stirred at RT for 10 min and poured into a solution of N-(5-fluoro-2-methyl-3-(6-(6-(3-(piperazin-1-yl)propoxy)pyridin-3-yl)-7H-pyrrolo[2,3-d]pyrimidin-4-yl)phenyl)-3-hydroxy-3-neopentylazetidine-1-carboxamide (39 mg, 0.047 mmol) in DMF (0.5 mL). The RM was stirred at RT for 20 min and poured into a mixture of EtOAc and H₂O. The aqueous layer was back-extracted with EtOAc/THF. Combined organic layers were washed with H₂O and with brine, dried over Na₂SO₄ and evaporated. The RM was purified by prep-TLC (Merck: PLC Silica gel 60 F₂₅₄, 0.5 mm, mobile phase: DCM:MeOH, 9:1), yielding the title compound (13 mg). Method LCMS5: Rt=3.85 min; [M+H]⁺=911.6. ¹H NMR (600 MHz, DMSO-d₆) δ 12.80 (d, J=2.2 Hz, 1H), 10.49 (s, 1H), 8.85 (s, 1H), 8.79 (d, J=2.5 Hz, 1H), 8.29 (m, 1H), 7.92 (s, 1H), 7.49-7.44 (m, 2H), 7.13 (d, J=3.0 Hz, 1H), 7.07 (m, 1H), 6.99-6.87 (m, 2H), 6.81 (d, J=2.0 Hz, 1H), 5.52 (s, 1H), 4.87 (s, 2H), 4.36 (t, J=6.6 Hz, 2H), 3.96 (m, 2H), 3.84 (m, 2H), 3.65 (m, 2H), 3.46 (m, 4H), 2.73 (m, 2H), 2.50-2.31 (m, 6H), 2.09 (s, 3H), 1.96-1.88 (m, 2H), 1.67 (s, 2H), 0.99 (s, 9H).

### Compound 14: N-(3-(6-(4-(((6-(3-(2,4-Dioxotetrahydropyrimidin-1(2H)-yl)phenoxy)hexyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

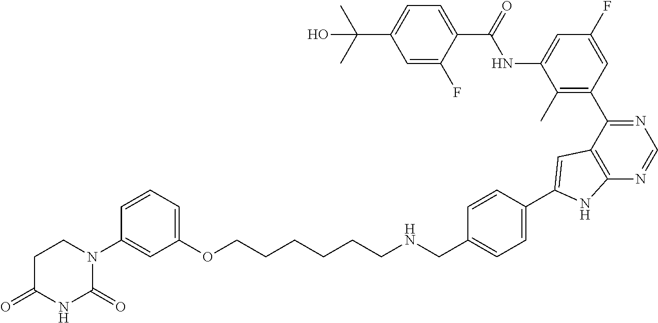 [SMILES: Cc1c(NC(=O)c2ccc(C(C)(C)O)cc2F)cc(F)cc1-c1ncnc2[nH]c(-c3ccc(CNCCCCCCOc4cccc(N5CCC(=O)NC5=O)c4)cc3)cc12]

To a mixture of 1-(3-((6-aminohexyl)oxy)phenyl)dihydropyrimidine-2,4(1H,3H)-dione (ILB-37, 85 mg, 0.244 mmol) and 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 154 mg, 0.292 mmol) was added at RT under argon THF (0.5 mL), MeOH (0.5 mL) and TEA (0.068 mL, 0.487 mmol). After stirring the mixture for 5 min at RT, a solution of zinc chloride (0.5 M) in THF (0.731 mL, 0.366 mmol) was added dropwise and stirring was continued at RT. After stirring for 5.5 h at RT, NaBH₃CN (16.85 mg, 0.268 mmol) was added in one portion and stirring was continued at RT overnight. Solvent was partially evaporated and then adsorbed on Isolute® and purified by reverse phase chromatography on a Redisep® Rf Gold 50 g HP C18 column eluting with ACN in an aq. solution of AcOH (0.5%) (from 2 to 100%) to afford, after filtration of the fraction 10 containing the pure target compound through PL-HCO₃ MP SPE cartridges and freeze drying, the title compound as a solid (41 mg). The fraction 11 also containing the expected compound (but not pure) was freeze dried yielding 27 mg of a slightly yellow solid which was then purified further by SFC Method XU on a Princeton PPU column (250×30 mm, 100 Å, 5 μm) eluting with methanol from 20% to 52% to afford the title compound (15 mg). Method LCMS1: Rt=0.86 min; [M+H]⁺=816.5. ¹H NMR (400 MHz, DMSO-d6) δ 12.71 (s, 1H), 10.33 (s, 1H), 9.93 (s, 1H), 8.84 (s, 1H), 7.92 (d, J=7.9 Hz, 2H), 7.73 (t, J=7.9 Hz, 1H), 7.66 (d, J=9.9 Hz, 1H), 7.45-7.34 (m, 4H), 7.28-7.18 (m, 2H), 6.92-6.72 (m, 4H), 5.29 (s, 1H), 3.94 (t, J=6.3 Hz, 2H), 3.78-3.66 (m, 4H), 2.66 (t, J=6.6 Hz, 3H), 2.16 (s, 3H), 1.69 (q, J=7.0 Hz, 2H), 1.50-1.30 (m, 14H).

### Compound 15: N-(3-(6-(4-((2-(((2-(3-(2,4-Dioxotetrahydropyrimidin-1(2H)-yl)phenoxy)ethyl)(methyl)amino)methyl)morpholino)methyl)phenyl)-7H-pyrrolo[2,3-d]pyrimidin-4-yl)-5-fluoro-2-methylphenyl)-2-fluoro-4-(2-hydroxypropan-2-yl)benzamide

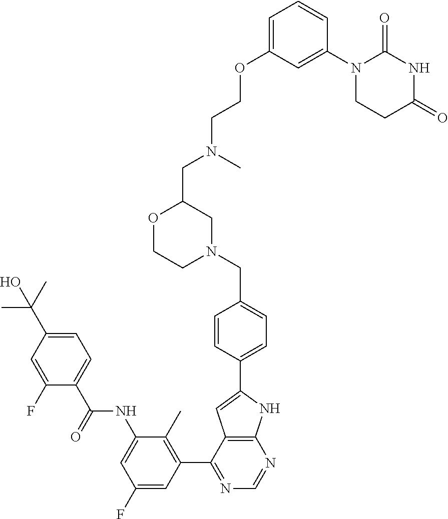 [SMILES: Cc1c(NC(=O)c2ccc(C(C)(C)O)cc2F)cc(F)cc1-c1ncnc2[nH]c(-c3ccc(CN4CCOC(CN(C)CCOc5cccc(N6CCC(=O)NC6=O)c5)C4)cc3)cc12]

### Step 1: tert-Butyl ((4-(4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)morpholin-2-yl)methyl)carbamate

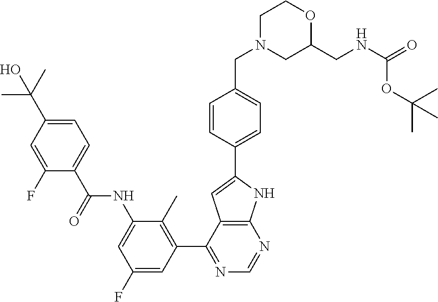 [SMILES: Cc1c(NC(=O)c2ccc(C(C)(C)O)cc2F)cc(F)cc1-c1ncnc2[nH]c(-c3ccc(CN4CCOC(CNC(=O)OC(C)(C)C)C4)cc3)cc12]

To a mixture of tert-butyl (morpholin-2-ylmethyl)carbamate (CAS No. [173341-02-1], 140 mg, 0.641 mmol) and 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 300 mg, 0.558 mmol) was added MeOH (5 mL) at RT under argon. After stirring the mixture for 5 min at RT, a solution of zinc chloride (0.5 M) in THF (1.3 mL, 0.650 mmol) was added and stirring was continued at RT. After stirring 6 h at RT, NaBH₃CN (40 mg, 0.637 mmol) was added in one portion and stirring was continued at RT overnight. Solvent was evaporated and the residue was purified by flash chromatography eluting with MeOH in DCM (from 0 to 18%) to afford the title compound as a yellow solid (406 mg). Method LCMS1: Rt=0.90 min; [M+H]⁺=727.4.

### Step 2: N-(3-(6-(4-((2-(Aminomethyl)morpholino)methyl)phenyl)-7H-pyrrolo[2,3-d]pyrimidin-4-yl)-5-fluoro-2-methylphenyl)-2-fluoro-4-(2-hydroxypropan-2-yl)benzamide

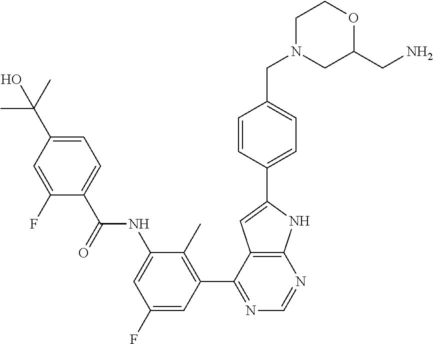 [SMILES: Cc1c(NC(=O)c2ccc(C(C)(C)O)cc2F)cc(F)cc1-c1ncnc2[nH]c(-c3ccc(CN4CCOC(CN)C4)cc3)cc12]

To tert-butyl ((4-(4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)morpholin-2-yl)methyl)carbamate (406 mg, 0.547 mmol) was added HCl (4.0 M) in dioxane (2.0 mL, 8.00 mmol). The resulting solution was stirred at RT for 2 h, then evaporated to dryness and further dried under HV overnight to afford the title compound as an HCl salt (409 mg). Method LCMS1: Rt=0.70 min; [M+H]⁺=627.4.

### Step 3: N-(3-(6-(4-((2-(((2-(3-(2,4-Dioxotetrahydropyrimidin-1(2H)-yl)phenoxy)ethyl)(methyl)amino)methyl)morpholino)methyl)phenyl)-7H-pyrrolo[2,3-d]pyrimidin-4-yl)-5-fluoro-2-methylphenyl)-2-fluoro-4-(2-hydroxypropan-2-yl)benzamide

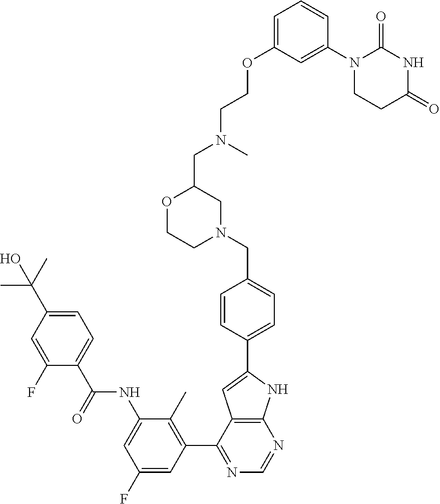 [SMILES: Cc1c(NC(=O)c2ccc(C(C)(C)O)cc2F)cc(F)cc1-c1ncnc2[nH]c(-c3ccc(CN4CCOC(CN(C)CCOc5cccc(N6CCC(=O)NC6=O)c5)C4)cc3)cc12]

N-(3-(6-(4-((2-(Aminomethyl)morpholino)methyl)phenyl)-7H-pyrrolo[2,3-d]pyrimidin-4-yl)-5-fluoro-2-methylphenyl)-2-fluoro-4-(2-hydroxypropan-2-yl)benzamide (116 mg, 0.156 mmol), TEA (0.050 mL, 0.359 mmol) and 2-(3-(2,4-dioxotetrahydropyrimidin-1(2H)-yl)phenoxy)acetaldehyde (ILB-18, 38.7 mg, 0.156 mmol) were dissolved in MeOH (1.5 mL) at RT. Zinc chloride (0.5 M) in THF (0.350 mL, 0.175 mmol) was added and the RM was stirred overnight at RT. Then NaBH₃CN (12 mg, 0.191 mmol) was added and the RM was stirred overnight at RT. More 2-(3-(2,4-dioxotetrahydropyrimidin-1(2H)-yl)phenoxy)acetaldehyde (ILB-13, 38.7 mg, 0.156 mmol) in THF (0.5 mL) was then added and the RM was stirred for 3 days at RT. The crude was purified by reverse phase chromatography on a Redisep® C18 column eluting with ACN in an aq. solution of TFA (0.1%) (from 2 to 90%) to afford, after filtration of the fractions containing the target compound through PL-HCO₃ MP SPE cartridges and freeze drying, 19 mg of the title compound (Purity 65%). It was then purified further by SFC using Method XU on a Torus 2PIC column (250×30 mm, 130 Å, 5 μm) eluting with methanol from 22% to 48% to afford the title compound as a white powder (3.4 mg). Method LCMS1: Rt=0.84 min; [M+H]⁺=873.4. ¹H NMR (400 MHz, DMSO-d6) δ 12.73 (s, 1H), 10.31 (s, 1H), 9.92 (s, 1H), 8.83 (s, 1H), 7.92 (d, J=8.0 Hz, 2H), 7.72 (t, J=7.8 Hz, 1H), 7.64 (d, J=9.9 Hz, 1H), 7.44-7.34 (m, 4H), 7.24-7.18 (m, 2H), 6.91-6.78 (m, 3H), 6.75 (d, J=8.4 Hz, 1H), 5.28 (s, 1H), 3.97 (t, J=6.3 Hz, 2H), 3.78-3.68 (m, 3H), 3.55 (m, 3H), 3.47 (m, 3H), 2.82-2.58 (m, 6H), 2.42 (m, 2H), 2.24 (s, 3H), 2.16 (s, 3H), 1.44 (s, 6H).

### Compound 16: N-(3-(6-(4-(((6-(3-(2,4-Dioxotetrahydropyrimidin-1(2H)-yl)phenoxy)hexyl)(methyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

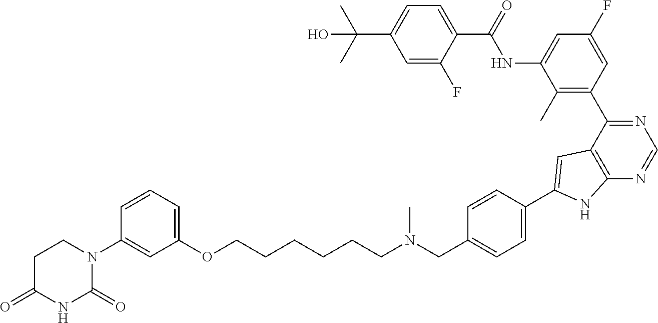 [SMILES: Cc1c(NC(=O)c2ccc(C(C)(C)O)cc2F)cc(F)cc1-c1ncnc2[nH]c(-c3ccc(CN(C)CCCCCCOc4cccc(N5CCC(=O)NC5=O)c4)cc3)cc12]

### Step 1: N-(3-(6-(4-(((6-(3-(2,4-Dioxotetrahydropyrimidin-1(2H)-yl)phenoxy)hexyl)(methyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

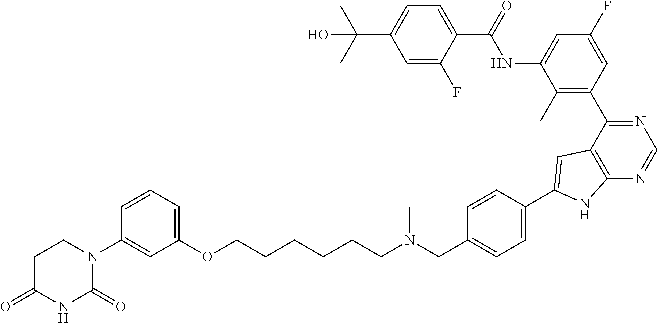 [SMILES: Cc1c(NC(=O)c2ccc(C(C)(C)O)cc2F)cc(F)cc1-c1ncnc2[nH]c(-c3ccc(CN(C)CCCCCCOc4cccc(N5CCC(=O)NC5=O)c4)cc3)cc12]

To a mixture of 1-(3-((6-(methylamino)hexyl)oxy)phenyl)dihydropyrimidine-2,4(1H,3H)-dione (ILB-54, 85 mg, 0.239 mmol) and 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 151 mg, 0.287 mmol) was added at RT under argon THF (0.5 mL), MeOH (0.5 mL) and TEA (0.067 mL, 0.478 mmol). After stirring the mixture for 5 min at RT, a solution of zinc chloride (0.5 M) in THF (0.717 mL, 0.358 mmol) was added dropwise and stirring was continued at RT. After stirring for 5.5 h at RT, NaBH₃CN (16.51 mg, 0.263 mmol) was added in one portion and stirring was continued at RT overnight. Solvent was partially evaporated and then adsorbed on Isolute® and purified by reverse phase chromatography on a Redisep® Rf Gold 50 g HP C18 column eluting with ACN in an aq. solution of AcOH (0.5%) (from 2 to 100% ACN) to afford, after freeze drying, the title compound as a acetic acid salt (123 mg). Method LCMS1: Rt=0.85 min; [M+H]⁺=830.5. ¹H NMR (400 MHz, DMSO-d6) δ 12.74 (s, 1H), 10.32 (s, 1H), 9.92 (s, 1H), 8.84 (s, 1H), 7.93 (d, J=7.8 Hz, 2H), 7.72 (t, J=7.9 Hz, 1H), 7.65 (d, J=10.1 Hz, 1H), 7.47-7.33 (m, 4H), 7.27-7.18 (m, 2H), 6.92-6.80 (m, 3H), 6.77 (dd, J=8.3, 2.2 Hz, 1H), 5.28 (s, 1H), 3.93 (t, J=6.4 Hz, 2H), 3.74 (t, J=6.6 Hz, 2H), 3.48 (s, 2H), 2.66 (t, J=6.6 Hz, 2H), 2.33 (m, 2H), 2.23-2.02 (m, 6H), 1.77-1.62 (m, 2H), 1.57-1.27 (m, 13H).

### Compound 17: 5-(2,4-Dioxotetrahydropyrimidin-1(2H)-yl)-N-(5-((4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)(methyl)amino)pentyl)-6-methylnicotinamide

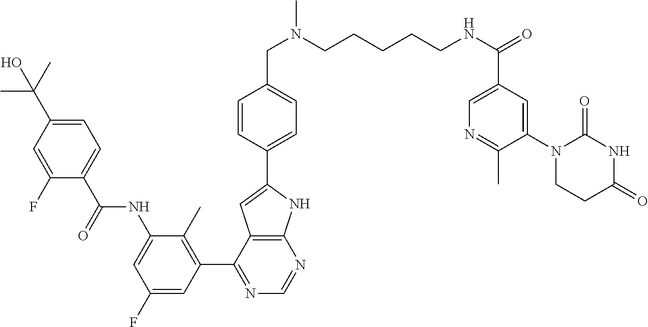 [SMILES: Cc1ncc(C(=O)NCCCCCN(C)Cc2ccc(-c3cc4c(-c5cc(F)cc(NC(=O)c6ccc(C(C)(C)O)cc6F)c5C)ncnc4[nH]3)cc2)cc1N1CCC(=O)NC1=O]

### Step 1: tert-Butyl (5-((4-(4-(5-Fluoro-3-(2-fluoro-4-(2-hydroxypropan-2-yl)benzamido)-2-methylphenyl)-7H-pyrrolo[2,3-d]pyrimidin-6-yl)benzyl)(methyl)amino)pentyl)carbamate

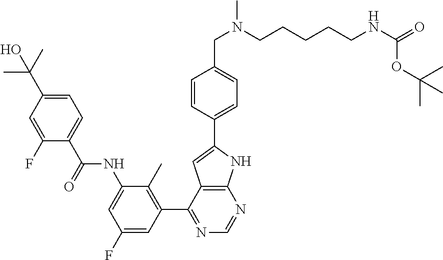 [SMILES: Cc1c(NC(=O)c2ccc(C(C)(C)O)cc2F)cc(F)cc1-c1ncnc2[nH]c(-c3ccc(CN(C)CCCCCNC(=O)OC(C)(C)C)cc3)cc12]

To a mixture of 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 300 mg, 0.570 mmol) and 5-(methylamino)-N-Boc-pentanamine (CAS [1311458-36-2], 140 mg, 0.627 mmol) in MeOH (12 mL) was added ZnCl₂ 0.5 M in THF (1.253 mL, 0.627 mmol). The resulting yellow mixture was flushed with N₂ and stirred at RT for 3 h. Then, NaBH₃CN (41.5 mg, 0.627 mmol) was added, and it was stirred at RT for 18 h. More 5-(methylamino)-N-Boc-pentanamine (25 mg, 0.112 mmol) in MeOH (1 mL), followed by more ZnCl₂ 0.5 M in THF (228 μL, 0.114 mmol) were added and the RM was stirred at RT. After 6 h, more NaBH₃CN (37 mg, 0.559 mmol) was added and the RM was stirred at RT for overnight. Then, more NaBH₃CN (19 mg, 0.287 mmol) was added, and it was stirred at RT for 6 h. Then, more 5-(methylamino)-N-Boc-pentanamine (64 mg, 0.287 mmol), followed by more ZnCl₂ 0.5 M in THF (570 μL, 0.285 mmol) were added and the RM was stirred at RT for overnight. The RM was concentrated until dryness and purified by flash chromatography on silica gel eluting with 10-70% (DCM/MeOH 80/20) in DCM to afford the title compound as a yellow solid (366 mg). Method LCMS1: Rt=0.92 min; [M+H]⁺=727.5.

### Step 2: N-(3-(6-(4-(((5-Aminopentyl)(methyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

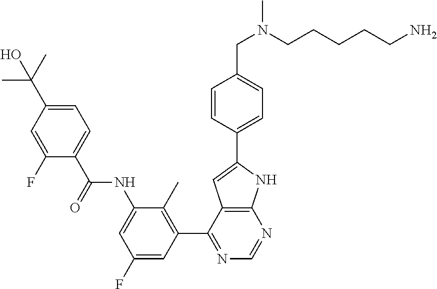 [SMILES: Cc1c(NC(=O)c2ccc(C(C)(C)O)cc2F)cc(F)cc1-c1ncnc2[nH]c(-c3ccc(CN(C)CCCCCN)cc3)cc12]

To a yellow mixture of tert-butyl (5-((4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)(methyl)amino)pentyl)carbamate (366 mg, 0.493 mmol) in dioxane (10 mL) was added HCl 4 M in dioxane (1.851 mL, 7.40 mmol). The resulting yellow mixture was then stirred at RT for 2.5 h, the RM was concentrated until dryness and dried under HV to afford the title compound as a yellow solid HCl salt (431 mg). Method LCMS1: Rt=0.66 min; [M+H]⁺=627.4.

### Step 3: 5-(2,4-Dioxotetrahydropyrimidin-1(2H)-yl)-N-(5-((4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)(methyl)amino)pentyl)-6-methylnicotinamide

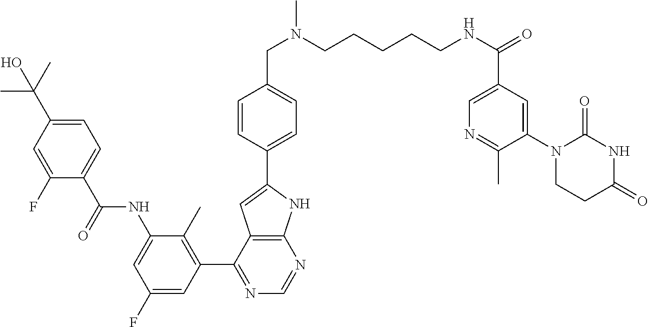 [SMILES: Cc1ncc(C(=O)NCCCCCN(C)Cc2ccc(-c3cc4c(-c5cc(F)cc(NC(=O)c6ccc(C(C)(C)O)cc6F)c5C)ncnc4[nH]3)cc2)cc1N1CCC(=O)NC1=O]

To a yellow solution of N-(3-(6-(4-(((5-aminopentyl)(methyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide (36 mg, 0.049 mmol), 5-(2,4-dioxotetrahydropyrimidin-1(2H)-yl)-6-methylnicotinic acid (ILB-30, 19.54 mg, 0.054 mmol) and HBTU (20.82 mg, 0.054 mmol) in dry DMF (1.2 mL) flushed with N₂ was added DIPEA (68 μL, 0.391 mmol). The resulting RM was stirred at RT for 1 h. The RM was stored in the freezer for overnight, then diluted with ACN, adsorbed on Isolute®, concentrated until dryness and dried under HV pump. It was purified by reverse phase chromatography on a Redisep® C18 column eluting with ACN in an aq. solution of TFA (0.1%) (from 10% to 100%) to afford, after filtration of the fractions containing the target compound through PL-HCO₃ MP SPE cartridges and freeze drying, a non-pure off-white solid. The solid was then purified by SFC Method XU (column: Princeton PPU 250×30 mm, 100 Å, 5 μM) eluting with 30-45% CO₂ in MeOH to afford the title compound as a white solid (5 mg). Method LCMS1: Rt=0.75 min; [M+H]⁺=858.4. ¹H NMR (400 MHz, DMSO-d6) δ 1.32-1.40 (m, 2H) 1.47 (s, 6H) 1.49-1.63 (m, 4H) 1.97-2.35 (m, 6H) 2.43 (s, 3H) 2.45-2.49 (m, 2H) 2.71-2.75 (m, 1H) 2.79-2.84 (m, 1H) 3.22-3.28 (m, 2H) 3.51 (m, J=10.00 Hz, 2H) 3.58-3.64 (m, 1H) 3.80-3.89 (m, 1H) 5.32 (s, 1H) 6.86 (br s, 1H) 7.26 (dd, J=8.74, 2.75 Hz, 1H) 7.32-7.49 (m, 4H) 7.67 (br d, J=10.15 Hz, 1H) 7.72-7.77 (m, 1H) 7.86-8.07 (m, 2H) 8.10 (d, J=1.83 Hz, 1H) 8.64 (t, J=1.00 Hz, 1H) 8.82-8.89 (m, 2H) 9.96 (s, 1H) 10.52 (s, 1H) 12.78 (br s, 1H).

### Compound 18: N-(3-(6-(4-(((5-(3-(2,4-dioxotetrahydropyrimidin-1(2H)-yl)-4-methylphenylsulfonamido)pentyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

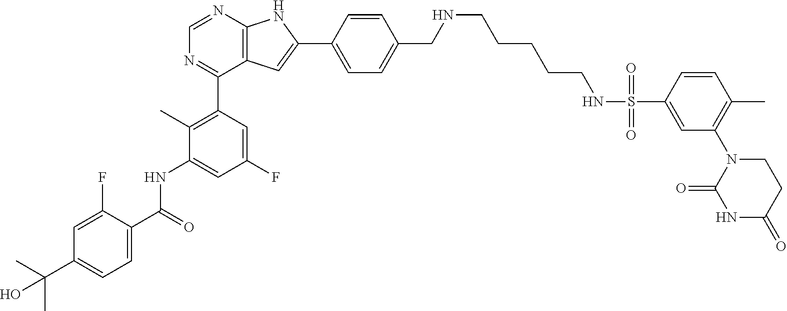 [SMILES: Cc1ccc(S(=O)(=O)NCCCCCNCc2ccc(-c3cc4c(-c5cc(F)cc(NC(=O)c6ccc(C(C)(C)O)cc6F)c5C)ncnc4[nH]3)cc2)cc1N1CCC(=O)NC1=O]

N-(5-aminopentyl)-3-(2,4-dioxotetrahydropyrimidin-1(2H)-yl)-4-methylbenzenesulfonamide TFA salt (ILB-53, 228 mg, 0.392 mmol), TEA (0.150 mL, 1.076 mmol) and 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 220 mg, 0.418 mmol) were dissolved in MeOH (3 mL). A solution of ZnCl₂ 0.5M in THF (1 mL, 0.500 mmol) was added and the RM was stirred at RT overnight. NaBH₃CN (30 mg, 0.477 mmol) was added and the RM was stirred at RT overnight. The RM was concentrated to dryness. The crude material was purified by reverse phase chromatography on a Redisep® C18 column eluting with ACN in an aqueous solution of TFA (0.10%) (from 2% to 100%). Fractions containing pure target compound were filtered through PL-HCO₃ MP SPE cartridges and freeze dried to afford crude material. The material was purified further by SFC Method XU on a Princeton PPU column (250×30 mm, 100 Å, 5 μm) eluting with MeOH from 35% to 52%, yielding the title compound (36 mg). Method LCMS1: Rt=0.79 min; [M+H]⁺=879.5.

### Compound 19: N-(3-(6-(4-(((6-(4-(3-(2,4-Dioxotetrahydropyrimidin-1(2H)-yl)phenyl)-1H-1,2,3-triazol-1-yl)hexyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

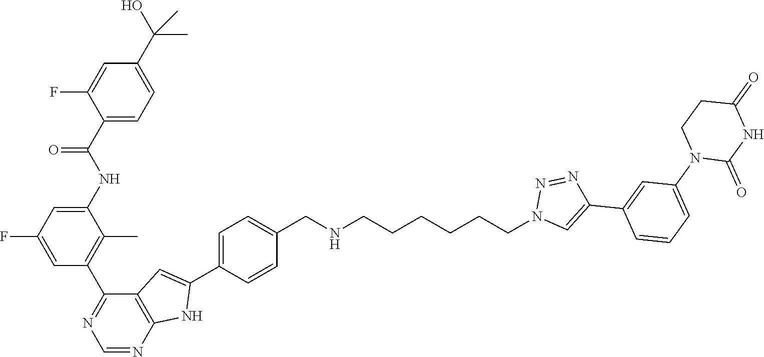 [SMILES: Cc1c(NC(=O)c2ccc(C(C)(C)O)cc2F)cc(F)cc1-c1ncnc2[nH]c(-c3ccc(CNCCCCCCn4cc(-c5cccc(N6CCC(=O)NC6=O)c5)nn4)cc3)cc12]

### Step 1: tert-Butyl (6-(4-(3-(2,4-dioxotetrahydropyrimidin-1(2H)-yl)phenyl)-1H-1,2,3-triazol-1-yl)hexyl)carbamate

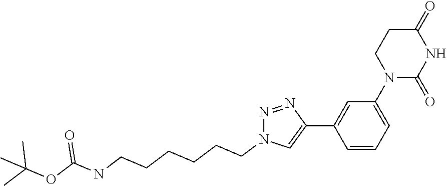

To a suspension of 1-(3-ethynylphenyl)dihydropyrimidine-2,4(1H,3H)-dione (ILB-33, 0.19 g, 0.579 mmol) and tert-butyl (6-azidohexyl)carbamate (CAS [129392-87-6], 0.154 g, 0.637 mmol) in THF (4 mL) and water (2 mL) was added sodium L-ascorbate (0.3M, 0.965 mL, 0.289 mmol) followed by Copper(II) sulfate pentahydrate (1M, 0.058 mL, 0.058 mmol). The reaction mixture was stirred at RT over 3 days. The reaction mixture was diluted with EtOAc and the organic phase was washed with aqueous NH₄OH solution and brine. The organic phase was dried over Na₂SO₄, filtered, and concentrated to dryness to afford the title compound as an oil (0.18 g). Method LCMS1: Rt=0.89 min; [M+H]⁺=457.3.

### Step 2: 1-(3-(1-(6-Aminohexyl)-1H-1,2,3-triazol-4-yl)phenyl)dihydropyrimidine-2,4(1H,3H)-dione

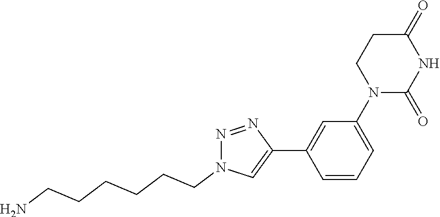 [SMILES: NCCCCCCn1cc(-c2cccc(N3CCC(=O)NC3=O)c2)nn1]

To a solution of tert-butyl (6-(4-(3-(2,4-dioxotetrahydropyrimidin-1(2H)-yl)phenyl)-1H-1,2,3-triazol-1-yl)hexyl)carbamate (0.18 g, 0.359 mmol) in DCM (3 mL) was added TFA (0.829 mL, 10.76 mmol). The reaction mixture was stirred at RT for 45 minutes. The reaction mixture was concentrated to dryness. The crude mixture purified by reverse phase preparative HPLC to afford the title compound as a TFA salt (0.04 g). Method LCMS1: Rt=0.46 min; [M+H]⁺=357.3.

### Step 3: N-(3-(6-(4-(((6-(4-(3-(2,4-dioxotetrahydropyrimidin-1(2H)-yl)phenyl)-1H-1,2,3-triazol-1-yl)hexyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

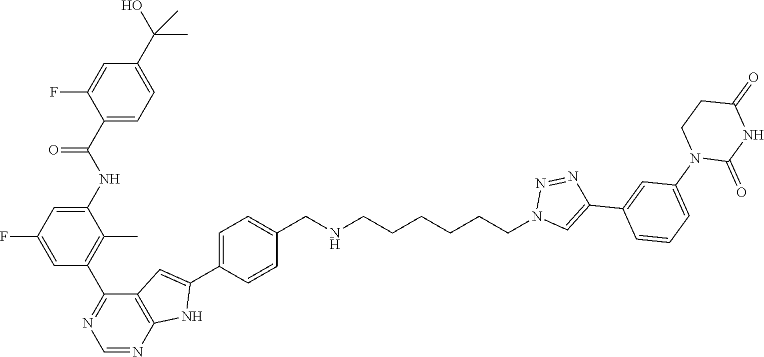 [SMILES: Cc1c(NC(=O)c2ccc(C(C)(C)O)cc2F)cc(F)cc1-c1ncnc2[nH]c(-c3ccc(CNCCCCCCn4cc(-c5cccc(N6CCC(=O)NC6=O)c5)nn4)cc3)cc12]

To a mixture of 1-(3-(1-(6-aminohexyl)-1H-1,2,3-triazol-4-yl)phenyl)dihydropyrimidine-2,4(1H,3H)-dione (0.041 g, 0.088 mmol), 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 0.06 g, 0.114 mmol) in MeOH (2 mL) and THF (1 mL, Ratio: 1.000) was added AcOH (0.015 mL, 0.263 mmol). The reaction mixture was stirred at room temperature for 7 h. NaBH₃CN (0.017 g, 0.263 mmol) was added and the reaction mixture was stirred at RT overnight. The reaction mixture was concentrated to dryness. The residue dissolved in MeOH/TFA and purified by reverse phase preparative HPLC (Method XN) to afford the title compound as a solid TFA salt. Method LCMS1: Rt=0.82 min; [M+H]⁺=867.6. ¹H NMR (600 MHz, DMSO-d₆) δ 12.88 (s, 1H), 10.44 (s, 1H), 9.99 (d, J=2.4 Hz, 1H), 8.90 (s, 1H), 8.70 (s, 2H), 8.63 (s, 1H), 8.08 (d, J=8.2 Hz, 2H), 7.82 (t, J=1.9 Hz, 1H), 7.72 (dt, J=24.5, 7.1 Hz, 3H), 7.58 (d, J=8.2 Hz, 2H), 7.53-7.36 (m, 2H), 7.34-7.22 (m, 1H), 6.97 (d, J=2.1 Hz, 1H), 5.33 (br s, 1H), 4.42 (t, J=6.9 Hz, 2H), 3.85 (t, J=6.7 Hz, 2H), 2.94 (d, J=14.3 Hz, 2H), 2.74 (t, J=6.6 Hz, 2H), 2.54-2.49 (m, 2H), 2.18 (s, 3H), 1.88 (t, J=7.4 Hz, 2H), 1.61 (s, 2H), 1.46 (s, 6H), 1.40-1.20 (m, 4H).

### Compound 20: N-(3-(6-(4-((4-(3-(4-(2,4-Dioxotetrahydropyrimidin-1(2H)-yl)phenyl)propyl)-1-oxa-4,9-diazaspiro[5.5]undecan-9-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

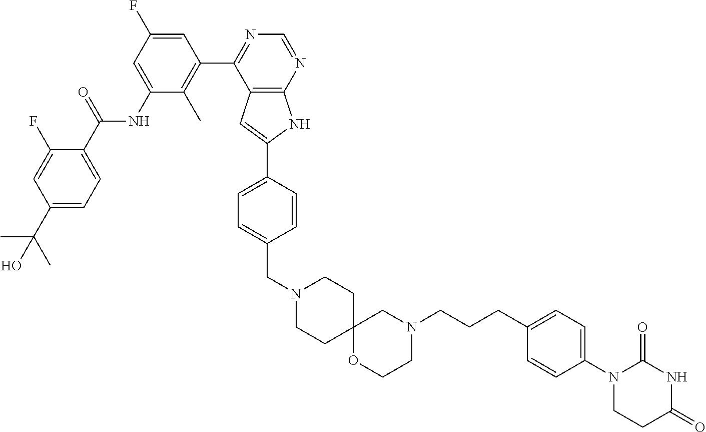 [SMILES: Cc1c(NC(=O)c2ccc(C(C)(C)O)cc2F)cc(F)cc1-c1ncnc2[nH]c(-c3ccc(CN4CCC5(CC4)CN(CCCc4ccc(N6CCC(=O)NC6=O)cc4)CCO5)cc3)cc12]

To a mixture of 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 135 mg, 0.26 mmol), 1-(4-(3-(1-oxa-4,9-diazaspiro[5.5]undecan-4-yl)propyl)phenyl)dihydropyrimidine-2,4(1H,3H)-dione (ILB-47, 90 mg, 0.23 mmol) in DMSO (3 mL) was added a solution of ZnCl₂ in THF (1 M, 0.35 mL, 0.35 mmol). The RM was stirred at 25° C. for 1 h then NaBH₃CN (147 mg, 2.33 mmol) and MeOH (1 mL) were added. The mixture was stirred at 40° C. for 6 h. The mixture was concentrated in vacuo. To the residue was added water (10 mL). A light yellow solid precipitated. The solid was filtered, dissolved in DCM/MeOH (1:1) and silica gel (100-200 mesh) was added. The mixture was concentrated in vacuo and purified by chromatography on a 12 g silica Biotage® column eluting with a methanolic ammonia solution (1N) in DCM (5-10%), 20 mL/min, to afford a crude product. The product was further purified by prep-TLC (silica, ammonia in MeOH (0.7 N)/DCM 1:8) to afford a white solid (40 mg). The crude product was purified by flash chromatography on a C18 column eluting with MeOH (10-80%) in an aq. solution of NH₄HCO₃ (10 mM) to afford the title compound as a white solid (31 mg). Method G: Rt=1.89 min; [M+H]⁺=897.4. ¹H NMR (500 MHz, DMSO-d6) δ 12.76 (s, 1H), 10.33 (s, 1H), 9.94 (s, 1H), 8.85 (s, 1H), 7.94 (d, J=8.0 Hz, 2H), 7.73 (t, J=7.9 Hz, 1H), 7.66 (d, J=9.5 Hz, 1H), 7.46-7.33 (m, 4H), 7.26-7.16 (m, 5H), 6.83 (s, 1H), 5.30 (s, 1H), 3.75 (t, J=6.7 Hz, 2H), 3.66-3.54 (m, 2H), 3.49 (s, 2H), 2.69 (t, J=6.7 Hz, 2H), 2.58 (t, J=7.6 Hz, 2H), 2.46-2.38 (m, 2H), 2.35-2.24 (m, 4H), 2.24-2.10 (m, 7H), 1.90-1.76 (m, 2H), 1.74-1.66 (m, 2H), 1.58-1.48 (m, 2H), 1.45 (s, 6H).

### Compound 21: 4-(dimethylamino)-3-((7-(3-(4-(4-(4-(4-(2,4-dioxotetrahydropyrimidin-1(2H)-yl)phenyl)but-3-yn-1-yl)piperazine-1-carbonyl)piperazin-1-yl)propoxy)quinazolin-4-yl)amino)-N-methylbenzenesulfonamide

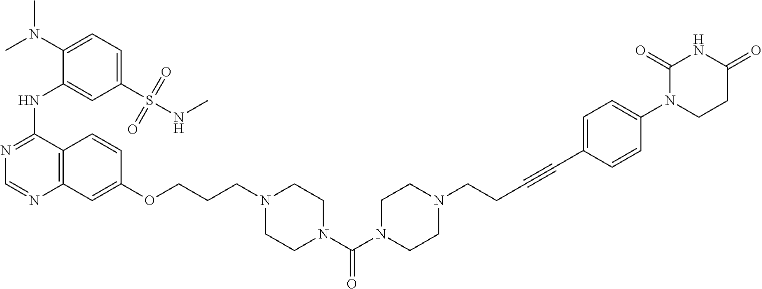 [SMILES: CNS(=O)(=O)c1ccc(N(C)C)c(Nc2ncnc3cc(OCCCN4CCN(C(=O)N5CCN(CCC#Cc6ccc(N7CCC(=O)NC7=O)cc6)CC5)CC4)ccc23)c1]

### Step 1: 4-(dimethylamino)-3-((7-(3-iodopropoxy)quinazolin-4-yl)amino)-N-methylbenzenesulfonamide

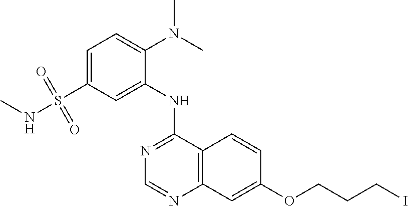 [SMILES: CNS(=O)(=O)c1ccc(N(C)C)c(Nc2ncnc3cc(OCCCI)ccc23)c1]

3-((7-(3-chloropropoxy)quinazolin-4-yl)amino)-4-(dimethylamino)-N-methylbenzenesulfonamide (intermediate CC, 1.00 g, 2.222 mmol) and NaI (0.666 g, 4.44 mmol) were suspended in acetone (20 mL) and refluxed for 72 hours. Upon heating, the reactants dissolve completely. The reaction mixture was cooled to RT and concentrated. The resulting solid was suspended in acetone (5 mL) and filtered, collecting the solid to afford the title compound as a yellow solid (1.20 g). Method XV: Rt=1.04 min; [M+H]⁺=542.9.

### Step 2: 4-(dimethylamino)-3-((7-(3-(4-(4-(4-(4-(2,4-dioxotetrahydropyrimidin-1(2H)-yl)phenyl)but-3-yn-1-yl)piperazine-1-carbonyl)piperazin-1-yl)propoxy)quinazolin-4-yl)amino)-N-methylbenzenesulfonamide

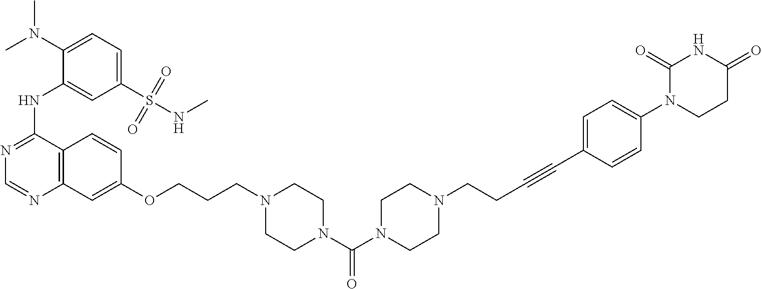 [SMILES: CNS(=O)(=O)c1ccc(N(C)C)c(Nc2ncnc3cc(OCCCN4CCN(C(=O)N5CCN(CCC#Cc6ccc(N7CCC(=O)NC7=O)cc6)CC5)CC4)ccc23)c1]

To a suspension of 1-(4-(4-(4-(piperazine-1-carbonyl)piperazin-1-yl)but-1-yn-1-yl)phenyl)dihydropyrimidine-2,4(1H,3H)-dione (ILB-41, 5 mg, 0.011 mmol) and 4-(dimethylamino)-3-((7-(3-iodopropoxy)quinazolin-4-yl)amino)-N-methylbenzenesulfonamide, (6.79 mg, 0.013 mmol) in DMA (114 μL) was added DIPEA (11.95 μL, 0.068 mmol). The resulting solution was stirred at 70° C. for 18 h. The reaction mixture was then cooled to RT, diluted with ACN and DMSO and purified via preparative HPLC (XBridge 30×50 mm 25-50% MeCN/H2O (5 mM NH₄OH)) to afford the title compound as a white powder (2.0 mg) Method XR: Rt=2.00 min; [M+H]⁺=853.5. ¹H NMR (400 MHz, DMSO-d6) δ 10.41 (s, 1H), 9.42 (s, 1H), 8.43 (s, 1H), 8.36 (d, J=8.9 Hz, 1H), 7.91 (d, J=2.4 Hz, 1H), 7.54 (d, J=6.7 Hz, 1H), 7.39 (d, J=8.4 Hz, 2H), 7.36-7.14 (m, 6H), 4.19 (s, 2H), 3.79 (t, J=6.5 Hz, 2H), 3.15 (s, 6H), 2.76 (s, 6H), 2.70 (s, 1H), 2.59 (s, 4H), 2.42 (d, J=5.1 Hz, 4H), 2.39 (s, 4H), 2.11-2.03 (m, 4H), 1.95 (s, 2H), 1.24 (s, 4H).

### Compound 22: 4-(dimethylamino)-3-((7-(3-(4-(3-(4-(2,4-dioxotetrahydropyrimidin-1(2H)-yl)phenyl)prop-2-yn-1-yl)-1-oxa-4,9-diazaspiro[5.5]undecan-9-yl)propoxy)quinazolin-4-yl)amino)-N-methylbenzenesulfonamide

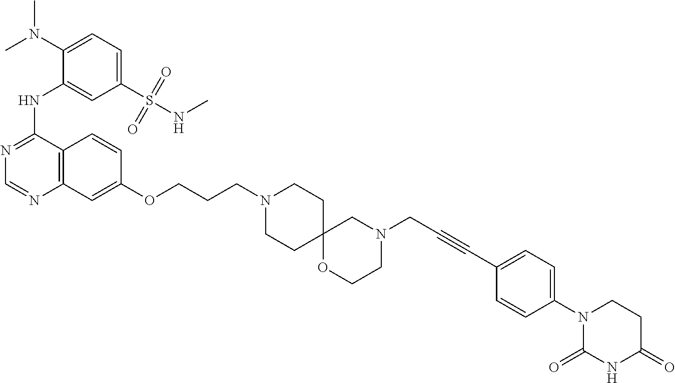 [SMILES: CNS(=O)(=O)c1ccc(N(C)C)c(Nc2ncnc3cc(OCCCN4CCC5(CC4)CN(CC#Cc4ccc(N6CCC(=O)NC6=O)cc4)CCO5)ccc23)c1]

To a suspension of 3-((7-(3-chloropropoxy)quinazolin-4-yl)amino)-4-(dimethylamino)-N-methylbenzenesulfonamide (intermediate CC, 34.4 mg, 0.076 mmol) and 1-(4-(3-(1-oxa-4,9-diazaspiro[5.5]undecan-4-yl)prop-1-yn-1-yl)phenyl)dihydropyrimidine-2,4(1H,3H)-dione (ILB-40, 32 mg, 0.076 mmol) in DMA (764 μL) was added potassium iodide (25.4 mg, 0.153 mmol) and DIPEA (50.6 μL, 0.290 mmol). The resulting solution was stirred at 80° C. for 72 h. The RM was diluted with ACN and DMSO and purified via preparative HPLC (XBridge 30×50 mm 10-30% MeCN/H2O (0.1% formic acid) and further via preparative HPLC (XBridge 30×50 mm 25-50% MeCN/H2O (5 mM NH₄OH) to afford the title compound as a white powder (15 mg). Method XR: Rt=2.19 min; [M+H]⁺=796.4. ¹H NMR (400 MHz, DMSO-d6) δ 10.42 (s, 1H), 9.42 (s, 1H), 8.43 (s, 1H), 8.35 (d, J=9.3 Hz, 1H), 7.92 (d, J=2.3 Hz, 1H), 7.53 (dd, J=8.4, 2.4 Hz, 1H), 7.50-7.42 (m, 2H), 7.38-7.31 (m, 2H), 7.27 (q, J=5.1 Hz, 1H), 7.25-7.14 (m, 3H), 4.18 (t, J=6.3 Hz, 2H), 3.80 (t, J=6.7 Hz, 2H), 3.65 (t, J=4.7 Hz, 2H), 3.49 (s, 2H), 3.30-3.27 (m, 3H), 2.76 (s, 6H), 2.70 (t, J=6.7 Hz, 2H), 2.42 (d, J=5.2 Hz, 6H), 2.37 (s, 2H), 2.09 (s, 2H), 2.01-1.89 (m, 2H), 1.89-1.79 (m, 2H), 1.57 (d, J=11.8 Hz, 2H).

### Compound 23: N-(3-(6-(4-((4-(4-(4-(4-(2,4-Dioxotetrahydropyrimidin-1(2H)-yl)phenyl)but-3-yn-1-yl)piperazine-1-carbonyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

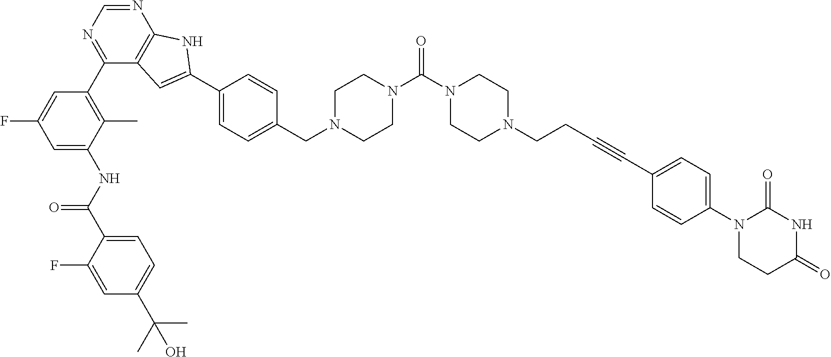 [SMILES: Cc1c(NC(=O)c2ccc(C(C)(C)O)cc2F)cc(F)cc1-c1ncnc2[nH]c(-c3ccc(CN4CCN(C(=O)N5CCN(CCC#Cc6ccc(N7CCC(=O)NC7=O)cc6)CC5)CC4)cc3)cc12]

A solution of 1-(4-(4-(4-(piperazine-1-carbonyl)piperazin-1-yl)but-1-yn-1-yl)phenyl)dihydropyrimidine-2,4(1H,3H)-dione (ILB-41, 420 mg, 0.63 mmol) and DIPEA (215 mg, 1.66 mmol) in MeOH (8 mL) and DMSO (3 mL) was stirred at 12° C. for 5 min. A solution of 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 216 mg, 0.41 mmol) in DMSO (5 mL) and ZnCl₂ (0.5 mL, 1 N in THF) was added at 12° C. The mixture was stirred at 22° C. for 2 h then NaBH₃CN (40 mg, 0.636 mmol) was added. The RM was stirred at 22° C. for 2 h then NaBH₃CN (22 mg 0.35 mmol) was added. The RM was stirred at 22° C. for 18 h. The RM (combined with the RM of a trial reaction) was concentrated under vacuum to give a DMSO solution which was purified by reverse phase chromatography eluting with ACN in an aq. solution of NH₄HCO₃ (10 mM) to give a crude product. Further purifications by reverse phase column chromatography eluting with ACN in an aq. solution of TFA (0.01%) and prep-HPLC using method PB (ACN in an aq. solution of NH₄HCO₃ (10 mM)) afforded the title product as a white solid (36 mg). Method H: Rt=1.893 min; [M+H]⁺=949. ¹H NMR (500 MHz, DMSO-d₆) δ 12.79 (s, 1H), 10.43 (s, 1H), 9.97 (s, 1H), 8.86 (s, 1H), 7.96 (d, J=8.2 Hz, 2H), 7.73 (t, J=8.0 Hz, 1H), 7.66 (d, J=8.3 Hz, 1H), 7.45-7.36 (m, 6H), 7.30 (d, J=8.5 Hz, 2H), 7.24 (dd, J=8.8, 2.5 Hz, 1H), 6.85 (s, 1H), 5.31 (s, 1H), 3.79 (t, J=6.6 Hz, 2H), 3.52 (s, 2H), 3.14 (s, 8H), 2.69 (t, J=6.6 Hz, 2H), 2.58 (s, 4H), 2.45-2.33 (m, 8H), 2.18 (s, 3H), 1.45 (s, 6H).

### Compound 24: N-(3-(6-(4-((4-((1-(4-(2,4-Dioxotetrahydropyrimidin-1(2H)-yl)-3-methoxybenzoyl)piperidin-4-yl)oxy)piperidin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

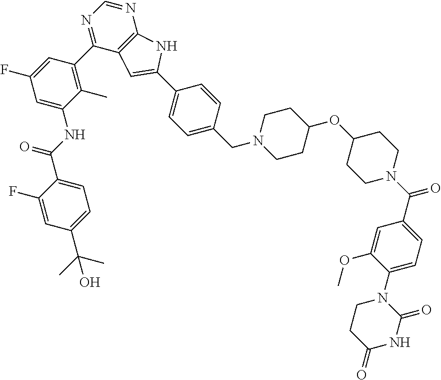 [SMILES: COc1cc(C(=O)N2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O]

### Step 1: tert-Butyl 4-((1-(4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)piperidin-4-yl)oxy)piperidine-1-carboxylate

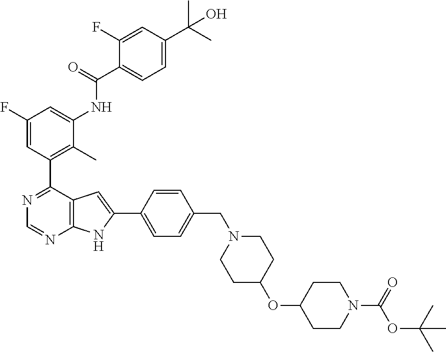 [SMILES: Cc1c(NC(=O)c2ccc(C(C)(C)O)cc2F)cc(F)cc1-c1ncnc2[nH]c(-c3ccc(CN4CCC(OC5CCN(C(=O)OC(C)(C)C)CC5)CC4)cc3)cc12]

tert-Butyl 4-(piperidin-4-yloxy)piperidine-1-carboxylate (CAS No. [845305-83-1], 106.5 mg, 0.374 mmol) and 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 190 mg, 0.361 mmol) were dissolved in MeOH (3 mL). A solution of ZnCl₂ 0.5M in THF (0.750 mL, 0.375 mmol) was added and the resulting solution was stirred at RT overnight. NaBH₃CN (24 mg, 0.382 mmol) was added and the RM was stirred at RT overnight. The RM was concentrated to dryness and the crude material was purified by silica gel chromatography eluting with MeOH in DCM (from 0% to 50%) to afford the title compound (285 mg). Method LCMS1: Rt=0.98 min; [M+H]⁺=795.5.

### Step 2: 2-Fluoro-N-(5-fluoro-2-methyl-3-(6-(4-((4-(piperidin-4-yloxy)piperidin-1-yl)methyl)phenyl)-7H-pyrrolo[2,3-d]pyrimidin-4-yl)phenyl)-4-(2-hydroxypropan-2-yl)benzamide

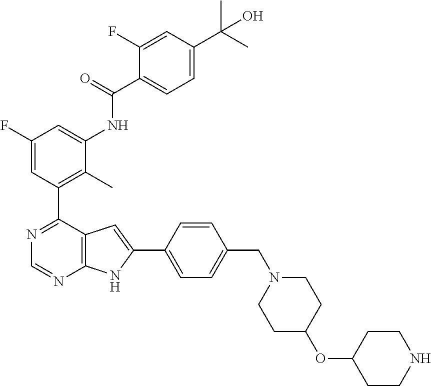 [SMILES: Cc1c(NC(=O)c2ccc(C(C)(C)O)cc2F)cc(F)cc1-c1ncnc2[nH]c(-c3ccc(CN4CCC(OC5CCNCC5)CC4)cc3)cc12]

tert-Butyl 4-((1-(4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)piperidin-4-yl)oxy)piperidine-1-carboxylate (269 mg, 0.332 mmol) was treated with HCl 4M in dioxane (1.5 mL, 6.00 mmol). MeOH (2 mL) was added and the RM was stirred at RT for 90 min. The RM was concentrated to dryness to afford the HCl salt of the title compound as a yellow solid (304 mg). Method LCMS1: Rt=0.66 min; [M+H]⁺=695.5.

### Step 3: N-(3-(6-(4-((4-((1-(4-(2,4-Dioxotetrahydropyrimidin-1(2H)-yl)-3-methoxybenzoyl)piperidin-4-yl)oxy)piperidin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

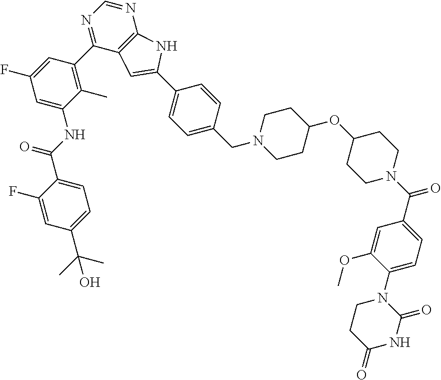 [SMILES: COc1cc(C(=O)N2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O]

4-(2,4-Dioxotetrahydropyrimidin-1(2H)-yl)-3-methoxybenzoic acid (ILB-81, 32 mg, 0.105 mmol) was dissolved in DMF (0.5 mL) to which NMM (0.025 mL, 0.227 mmol) was added followed by HATU (44 mg, 0.116 mmol). A solution of 2-fluoro-N-(5-fluoro-2-methyl-3-(6-(4-((4-(piperidin-4-yloxy)piperidin-1-yl)methyl)phenyl)-7H-pyrrolo[2,3-d]pyrimidin-4-yl)phenyl)-4-(2-hydroxypropan-2-yl)benzamide HCl salt (90 mg, 0.100 mmol) and NMM (0.025 mL, 0.227 mmol) in DMF (0.5 mL) was added dropwise. The RM was stirred at RT for 90 min. The crude RM was purified by reverse phase chromatography on a RediSep® C18 column eluting with ACN in an aqueous solution of TFA (0.1%) (from 5% to 100%). Fractions containing pure target compound were filtered through PL-HCO₃ MP SPE cartridges and freeze dried to afford the title compound (62 mg). Method LCMS5: Rt=3.72 min; [M+H]⁺=941.4. ¹H NMR (400 MHz, DMSO-d₆) δ 12.73 (s, 1H), 10.32 (s, 1H), 9.92 (s, 1H), 8.84 (s, 1H), 7.92 (d, J=7.9 Hz, 2H), 7.72 (m, 1H), 7.64 (m, 1H), 7.44-7.34 (m, 4H), 7.28 (d, J=7.9 Hz, 1H), 7.22 (d, J=8.7 Hz, 1H), 7.07 (s, 1H), 6.95 (d, J=7.7 Hz, 1H), 6.81 (s, 1H), 5.28 (s, 1H), 3.80 (s, 3H), 3.72-3.63 (m, 1H), 3.58 (t, J=6.6 Hz, 2H), 3.53-3.39 (m, 3H), 2.66 (m, 6H), 2.16 (s, 3H), 2.08 (m, 4H), 1.90-1.68 (m, 4H), 1.51-1.33 (m, 10H).

### Compound 25: N-(3-(6-(4-((4-(4-(4-(2,4-dioxotetrahydropyrimidin-1(2H)-yl)-3-methoxybenzamido)butyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

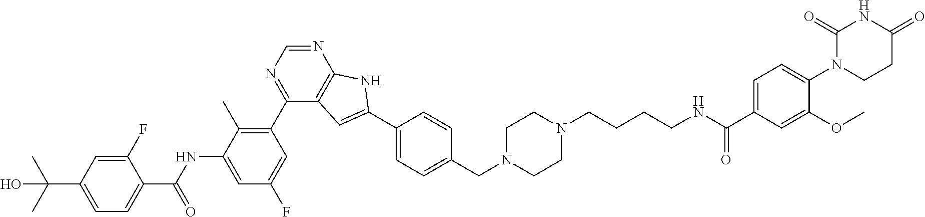 [SMILES: COc1cc(C(=O)NCCCCN2CCN(Cc3ccc(-c4cc5c(-c6cc(F)cc(NC(=O)c7ccc(C(C)(C)O)cc7F)c6C)ncnc5[nH]4)cc3)CC2)ccc1N1CCC(=O)NC1=O]

At RT, in a 10 mL round-bottomed flask, 4-(2,4-dioxotetrahydropyrimidin-1(2H)-yl)-3-methoxy-N-(4-(piperazin-1-yl)butyl)benzamide (intermediate FF, 103 mg, 0.205 mmol), TEA (0.050 mL, 0.359 mmol) and 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 110 mg, 0.205 mmol) were dissolved in MeOH (2 mL). ZnCl₂ 0.5M in THF (0.5 mL, 0.250 mmol) was added and the RM was stirred overnight at RT. Then NaBH₃CN (15 mg, 0.239 mmol) was added and RM was stirred overnight at RT. The reaction was evaporated. The crude product was loaded on a Redisep® C18 column and eluted from (water+0.1% TFA)/ACN 98:2 to 1:9. The desired fractions were collected and filtered over a PL-HCO3 MP SPE cartridge and lyophilized. Further purification by SFC (250×30 Reprospher PEI 100 A 5 um, 33 to 50% in 10 min), Method XU to afford the title compound (88 mg) as a white powder. Method LCMS6: Rt=0.78 min; [M+H]⁺=915. ¹H NMR (400 MHz, DMSO-d₆) δ 12.75 (s, 1H), 10.33 (s, 1H), 9.93 (m, 1H), 8.84 (m, 1H), 8.48 (m, 1H), 7.93 (m, 2H), 7.48 (m, 9H), 6.82 (s, 1H), 5.30 (s, 1H), 3.84 (m, 3H), 3.58 (m, 2H), 3.26 (m, 5H), 2.67 (m, 2H), 2.47-2.13 (m, 13H), 1.45 (m, 10H).

### Compound 26: N-(3-(6-(4-((4-(2-(2-(4-(2,4-Dioxotetrahydropyrimidin-1(2H)-yl)phenoxy)acetamido)ethyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

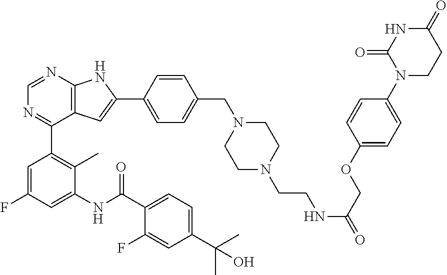 [SMILES: Cc1c(NC(=O)c2ccc(C(C)(C)O)cc2F)cc(F)cc1-c1ncnc2[nH]c(-c3ccc(CN4CCN(CCNC(=O)COc5ccc(N6CCC(=O)NC6=O)cc5)CC4)cc3)cc12]

### Step 1: tert-Butyl (2-(4-(4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)piperazin-1-yl)ethyl)carbamate

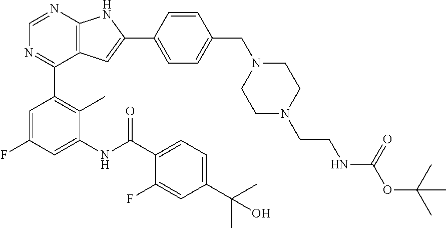 [SMILES: Cc1c(NC(=O)c2ccc(C(C)(C)O)cc2F)cc(F)cc1-c1ncnc2[nH]c(-c3ccc(CN4CCN(CCNC(=O)OC(C)(C)C)CC4)cc3)cc12]

1-2-N-Boc-(2-aminoethyl)piperazine (103 mg, 0.450 mmol), TEA (0.100 mL, 0.717 mmol) and 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 220 mg, 0.409 mmol) were dissolved in MeOH (4 mL). A solution of ZnCl₂ 0.5M in THF (1 mL, 0.500 mmol) was added and the resulting RM was stirred at RT overnight. NaBH₃CN (30 mg, 0.477 mmol) was added and the RM was stirred at RT overnight. The RM was concentrated to dryness. The residue was purified by chromatography on silica gel eluting with MeOH in DCM (from 0% to 50%) yielding the title compound (363 mg). Method LCMS1: Rt=0.89 min; [M+H]⁺=740.6.

### Step 2: N-(3-(6-(4-((4-(2-Aminoethyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

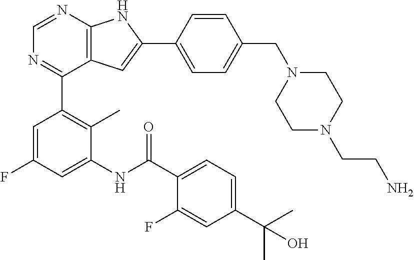 [SMILES: Cc1c(NC(=O)c2ccc(C(C)(C)O)cc2F)cc(F)cc1-c1ncnc2[nH]c(-c3ccc(CN4CCN(CCN)CC4)cc3)cc12]

To tert-butyl (2-(4-(4-(4-(5-fluoro-3-(2-fluoro-4-(2-hydroxypropan-2-yl)benzamido)-2-methylphenyl)-7H-pyrrolo[2,3-d]pyrimidin-6-yl)benzyl)piperazin-1-yl)ethyl)carbamate (353 mg, 0.382 mmol) was added a solution of HCl 4M in dioxane (2 mL, 8.00 mmol) and MeOH (2 mL). The RM was stirred at RT for 2 h and concentrated to dryness to afford the HCl salt of the title compound as a yellow solid (358 mg). Method LCMS1: Rt=0.65 min; [M+H]⁺=640.4.

### Step 3: N-(3-(6-(4-((4-(2-(2-(4-(2,4-Dioxotetrahydropyrimidin-1(2H)-yl)phenoxy)acetamido)ethyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

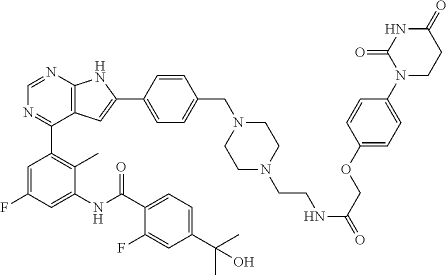 [SMILES: Cc1c(NC(=O)c2ccc(C(C)(C)O)cc2F)cc(F)cc1-c1ncnc2[nH]c(-c3ccc(CN4CCN(CCNC(=O)COc5ccc(N6CCC(=O)NC6=O)cc5)CC4)cc3)cc12]

To a solution of 2-(4-(2,4-dioxotetrahydropyrimidin-1(2H)-yl)phenoxy)acetic acid (ILB-14, 33 mg, 0.124 mmol) in DMF (0.5 mL) was added NMM (0.025 mL, 0.227 mmol), then HATU (47 mg, 0.124 mmol). A solution of N-(3-(6-(4-((4-(2-aminoethyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide HCl salt (100 mg, 0.112 mmol) and NMM (0.025 mL, 0.227 mmol) in DMF (0.5 mL) was added dropwise into the mixture. The RM was stirred at RT for 90 min. The crude RM was purified by reverse phase chromatography on a RediSep® C18 column eluting with ACN in an aqueous solution of TFA (0.1%) (from 2% to 100%). Fractions containing pure target compound were filtered through PL-HCO₃ MP SPE cartridges and freeze dried to afford the title compound (70 mg). Method LCMS5: Rt=3.68 min; [M+H]⁺=886.5. ¹H NMR (400 MHz, DMSO-d₆) δ 12.79 (s, 1H), 10.33 (s, 1H), 9.97 (s, 1H), 8.87 (s, 1H), 8.01-7.86 (m, 3H), 7.75 (m, 1H), 7.67 (m, 1H), 7.49-7.34 (m, 4H), 7.27-7.18 (m, 3H), 6.98 (d, J=8.5 Hz, 2H), 6.86 (s, 1H), 5.32 (s, 1H), 4.49 (s, 2H), 3.73 (t, J=6.7 Hz, 2H), 3.60-3.42 (m, 2H), 3.31-2.81 (m, 8H), 2.70 (m, 2H), 2.46-2.25 (m, 4H), 2.19 (s, 3H), 1.47 (s, 6H).

### Compound 27: N-(3-(6-(1-(5-(4-(4-(2,4-Dioxotetrahydropyrimidin-1(2H)-yl)phenoxy)piperidin-1-yl)pentanoyl)-1,2,3,6-tetrahydropyridin-4-yl)-7H-pyrrolo[2,3-d]pyrimidin-4-yl)-5-fluoro-2-methylphenyl)-2-fluoro-4-(2-hydroxypropan-2-yl)benzamide

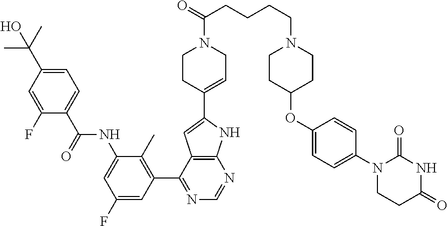 [SMILES: Cc1c(NC(=O)c2ccc(C(C)(C)O)cc2F)cc(F)cc1-c1ncnc2[nH]c(C3=CCN(C(=O)CCCCN4CCC(Oc5ccc(N6CCC(=O)NC6=O)cc5)CC4)CC3)cc12]

### Step 1: 4-chloro-6-iodo-7-tosyl-7H-pyrrolo[2,3-d]pyrimidine

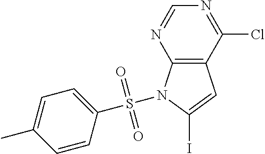 [SMILES: Cc1ccc(S(=O)(=O)n2c(I)cc3c(Cl)ncnc32)cc1]

At RT, 4-chloro-6-iodo-7H-pyrrolo[2,3-d]pyrimidine (CAS [876343-10-1], 11 g, 37.4 mmol) was suspended in DMF (40 mL) to give a white suspension. At 0° C., NaH 60% in mineral oil (1.795 g, 44.9 mmol) was added portionwise, resulting in a brown solution. After 15 min, p-toluenesulfonyl chloride (7.2 g, 37.4 mmol) was added portionwise, and the resulting red-brown RM was stirred for 4 h at RT. The RM was poured onto ice and water, then stirred overnight. The suspension was filtered, the solid was washed several times with a mixture of Et₂O/H₂O/ACN to afford the title compound as a beige solid (14.56 g). Method LCMS1: Rt=1.23 min; [M+H]⁺=434.0.

### Step 2: tert-Butyl 4-(4-chloro-7-tosyl-7H-pyrrolo[2,3-d]pyrimidin-6-yl)-5,6-dihydropyridine-1(2H)-carboxylate

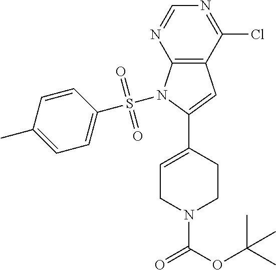 [SMILES: Cc1ccc(S(=O)(=O)n2c(C3=CCN(C(=O)OC(C)(C)C)CC3)cc3c(Cl)ncnc32)cc1]

To a solution of 4-chloro-6-iodo-7-tosyl-7H-pyrrolo[2,3-d]pyrimidine (9.5 g, 21.47 mmol) and 3,6-dihydro-2H-pyridine-1-N-Boc-4-boronic acid pinacol ester (CAS No. [286961-14-6], 8 g, 25.4 mmol) in iPrOH (120 mL) was added aq. 2M Na₂CO₃ (43 mL, 86 mmol). The mixture was degassed with argon, then PdCl₂(PPh₃)₂ (1.5 g, 2.116 mmol) was added and the RM was heated at 75° C. for 2 h. The RM was cooled down to RT, filtered over Celite® filter aid and washed with EtOAc. The resulting filtrate was diluted with water and extracted. The organic layer was washed with brine, dried over MgSO₄, and evaporated. The crude product was purified by flash chromatography on silica gel eluting with 0-45% EtOAc in CHX to afford an orange residue. The residue was triturated in Et₂O and filtered to afford the title compound as a beige powder (6.832 g). Method LCMS1: Rt=1.38 min; [M+H]⁺=489.2.

### Step 3: tert-Butyl 4-(4-(5-fluoro-3-(2-fluoro-4-(2-hydroxypropan-2-yl)benzamido)-2-methylphenyl)-7-tosyl-7H-pyrrolo[2,3-d]pyrimidin-6-yl)-5,6-dihydropyridine-1(2H)-carboxylate

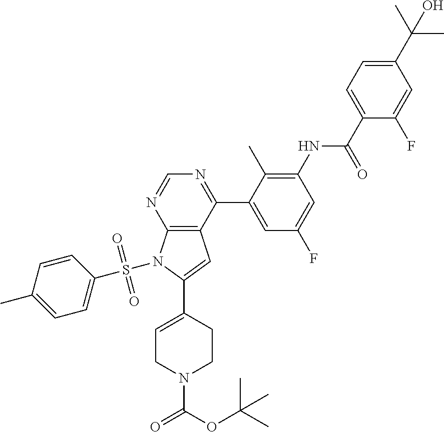 [SMILES: Cc1ccc(S(=O)(=O)n2c(C3=CCN(C(=O)OC(C)(C)C)CC3)cc3c(-c4cc(F)cc(NC(=O)c5ccc(C(C)(C)O)cc5F)c4C)ncnc32)cc1]

To a solution of tert-butyl 4-(4-chloro-7-tosyl-7H-pyrrolo[2,3-d]pyrimidin-6-yl)-5,6-dihydropyridine-1(2H)-carboxylate (7.915 g, 15.145 mmol) and 2-fluoro-N-(5-fluoro-2-methyl-3-(4,4,5,5-tetramethyl-1,3,2-dioxaborolan-2-yl)phenyl)-4-(2-hydroxypropan-2-yl)benzamide (intermediate 2 in PCT/IB2019/052346 7.998 g, 18.174 mmol) in DME (150 mL) was added aq. 1M Na₂CO₃ (45.4 mL, 45.4 mmol). The mixture was degassed with argon, PdCl₂(PPh₃)₂ (1.074 g, 1.5145 mmol) was added and the RM was heated at 100° C. for 1 h. The RM was cooled down to RT, filtered over Celite® filter aid, and washed with EtOAc. The filtrate was diluted with water and extracted. The organic layer was washed with brine, dried over MgSO₄, and evaporated. The crude product was purified by flash chromatography on silica gel eluting with 0-65% EtOAc in CHX to afford the title compound as an orange foam (13.52 g). Method LCMS1: Rt=1.36 min; [M+H]⁺=758.4.

### Step 4: 2-fluoro-N-(5-fluoro-2-methyl-3-(6-(1,2,3,6-tetrahydropyridin-4-yl)-7-tosyl-7H-pyrrolo[2,3-d]pyrimidin-4-yl)phenyl)-4-(2-hydroxypropan-2-yl)benzamide

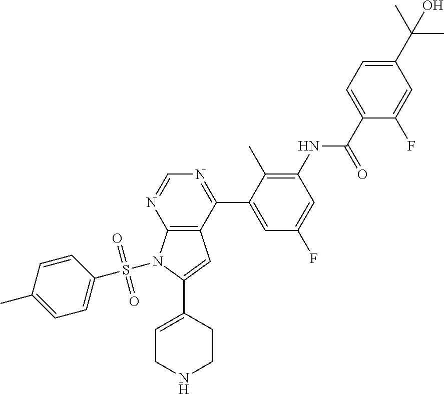 [SMILES: Cc1ccc(S(=O)(=O)n2c(C3=CCNCC3)cc3c(-c4cc(F)cc(NC(=O)c5ccc(C(C)(C)O)cc5F)c4C)ncnc32)cc1]

To a solution of tert-butyl 4-(4-(5-fluoro-3-(2-fluoro-4-(2-hydroxypropan-2-yl)benzamido)-2-methylphenyl)-7-tosyl-7H-pyrrolo[2,3-d]pyrimidin-6-yl)-5,6-dihydropyridine-1(2H)-carboxylate (13.52 g, 14.99 mmol) in dioxane (50 mL) was added HCl 4 M in dioxane (25 mL, 100 mmol). The orange solution was stirred overnight at RT, then the RM was evaporated to dryness. The resulting residue was diluted in EtOH, a few mL of cold MTBE were added and after trituration, the mixture was filtered. The crude product was purified by reverse phase chromatography on a Redisep® C18 column eluting with ACN in an aq. solution of TFA (0.1%) (from 10 to 100%). The fractions were evaporated, then partitioned between sat. aq. NaHCO₃ solution and DCM. After extraction, the solution was evaporated and dried under HV to afford the title compound as a white solid (7.8 g). Method LCMS1: Rt=0.88 min; [M+H]⁺=658.3.

### Step 5: 2-fluoro-N-(5-fluoro-2-methyl-3-(6-(1,2,3,6-tetrahydropyridin-4-yl)-7H-pyrrolo[2,3-d]pyrimidin-4-yl)phenyl)-4-(2-hydroxypropan-2-yl)benzamide

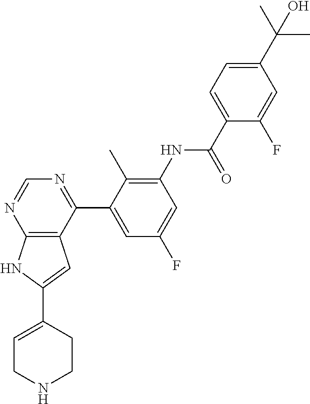 [SMILES: Cc1c(NC(=O)c2ccc(C(C)(C)O)cc2F)cc(F)cc1-c1ncnc2[nH]c(C3=CCNCC3)cc12]

To an orange solution of 2-fluoro-N-(5-fluoro-2-methyl-3-(6-(1,2,3,6-tetrahydropyridin-4-yl)-7-tosyl-7H-pyrrolo[2,3-d]pyrimidin-4-yl)phenyl)-4-(2-hydroxypropan-2-yl)benzamide (1.29 g, 1.961 mmol) in THF (19.5 mL) was added aq. 32% NaOH (363 μL, 3.92 mmol) and the RM was stirred at RT. After 3 nights at RT, the RM was concentrated until dryness to afford an orange resin. The resin was diluted with ACN and water, then TFA (302 μL, 3.92 mmol) was carefully added. The resulting solution was adsorbed on Isolute®, concentrated until dryness, dried under HV and purified by reverse phase chromatography on a Redisep® C18 column eluting with ACN in an aq. solution of TFA (0.1%) to afford, after freeze drying, the title compound as a yellow solid TFA salt (830 mg). Method LCMS1: Rt=0.67 min; [M+H]⁺=504.3.

### Step 6: tert-Butyl 5-(4-(4-(2,4-dioxotetrahydropyrimidin-1(2H)-yl)phenoxy)piperidin-1-yl)pentanoate

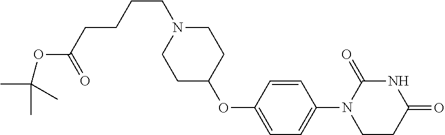 [SMILES: CC(C)(C)OC(=O)CCCCN1CCC(Oc2ccc(N3CCC(=O)NC3=O)cc2)CC1]

To a colorless solution of 1-(4-(piperidin-4-yloxy)phenyl)dihydropyrimidine-2,4(1H,3H)-dione (ILB-36, 123 mg, 0.305 mmol), tert-butyl 5-oxopentanoate (CAS [192123-41-4], 60.8 mg, 0.335 mmol) and TEA (42 μL, 0.305 mmol) in MeOH (6 mL) was added ZnCl₂ 0.5 M in THF (671 μL, 0.335 mmol). The resulting mixture was flushed with N₂ and stirred at RT. After 3 h at RT, NaBH₃CN (21.1 mg, 0.335 mmol) was added, and the RM was stirred at RT for 18 h. Then more tert-butyl 5-oxopentanoate (17 mg, 0.094 mmol) in MeOH (0.2 mL), followed by more ZnCl₂ 0.5 M in THF (183 μL, 0.091 mmol) were added, the resulting RM was stirred at RT for 2 h, before more NaBH₃CN (14 mg, 0.222 mmol) was added. The resulting RM was then stirred at RT for overnight, diluted with ACN and concentrated until dryness to give a colorless resin. The crude product was diluted with a mixture of DCM/MeOH/ACN, adsorbed on Isolute®, concentrated until dryness and purified by flash chromatography on silica gel eluting with 5-80% (DCM/iPrOH 80/20) in DCM to afford the title compound as a colorless resin (118 mg). Method LCMS1: Rt=0.68 min; [M+H]⁺=446.4.

### Step 7: 5-(4-(4-(2,4-dioxotetrahydropyrimidin-1(2H)-yl)phenoxy)piperidin-1-yl)pentanoic acid

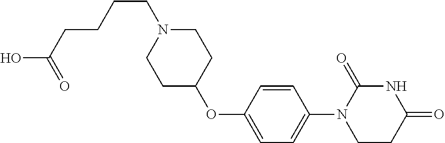 [SMILES: O=C(O)CCCCN1CCC(Oc2ccc(N3CCC(=O)NC3=O)cc2)CC1]

To a colorless solution of tert-butyl 5-(4-(4-(2,4-dioxotetrahydropyrimidin-1(2H)-yl)phenoxy)piperidin-1-yl)pentanoate (117 mg, 0.197 mmol) in DCM (2.8 mL) was added TFA (455 μL, 5.91 mmol). The resulting solution was stirred at RT for 1 h. The RM was diluted with DCM, concentrated until dryness, then co-evaporated with DCM (1×), and dried under HV pump to afford a colorless resin. The resin was freeze dried to afford an off-white solid. The solid was dissolved in ACN, adsorbed on Isolute®, concentrated until dryness and purified by reverse phase chromatography on a Redisep® C18 column eluting with ACN in an aq. solution of TFA (0.1%) (from 2 to 100%) to afford the title compound as a white solid TFA salt (62 mg). Method LCMS1: Rt=0.42 min; [M+H]⁺=390.3.

### Step 8: N-(3-(6-(1-(5-(4-(4-(2,4-dioxotetrahydropyrimidin-1(2H)-yl)phenoxy)piperidin-1-yl)pentanoyl)-1,2,3,6-tetrahydropyridin-4-yl)-7H-pyrrolo[2,3-d]pyrimidin-4-yl)-5-fluoro-2-methylphenyl)-2-fluoro-4-(2-hydroxypropan-2-yl)benzamide

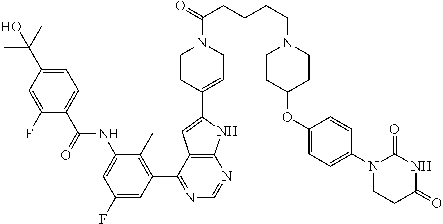 [SMILES: Cc1c(NC(=O)c2ccc(C(C)(C)O)cc2F)cc(F)cc1-c1ncnc2[nH]c(C3=CCN(C(=O)CCCCN4CCC(Oc5ccc(N6CCC(=O)NC6=O)cc5)CC4)CC3)cc12]

To a yellow solution of 2-fluoro-N-(5-fluoro-2-methyl-3-(6-(1,2,3,6-tetrahydropyridin-4-yl)-7H-pyrrolo[2,3-d]pyrimidin-4-yl)phenyl)-4-(2-hydroxypropan-2-yl)benzamide (45 mg, 0.062 mmol), 5-(4-(4-(2,4-dioxotetrahydropyrimidin-1(2H)-yl)phenoxy)piperidin-1-yl)pentanoic acid (34.1 mg, 0.068 mmol) and HBTU (26.2 mg, 0.068 mmol) in dry DMF (1.2 mL) flushed with N₂ was added DIPEA (75 μL, 0.431 mmol). The resulting RM was stirred at RT for 1 h, diluted with ACN, adsorbed on Isolute®, concentrated until dryness, then dried under HV and purified by reverse phase chromatography on a Redisep® C18 column eluting with ACN in an aq. solution of TFA (0.1%) (from 10 to 100%) to afford, after filtration of the fractions containing the pure target compound through PL-HCO₃ MP SPE cartridges and freeze drying, the title compound as a white solid (38 mg). Method LCMS1: Rt=0.77 min; [M+H]⁺=875.6. Method LCMS5: Rt=3.64 min; [M+H]+=875.6. ¹H NMR (400 MHz, DMSO-d₆) δ 1.44 (s, 8H) 1.48-1.62 (m, 4H) 1.89 (br d, J=10.64 Hz, 2H) 2.10-2.18 (m, 5H) 2.27 (br t, J=6.72 Hz, 2H) 2.33-2.41 (m, 2H) 2.45 (br s, 1H) 2.55 (br s, 1H) 2.62-2.69 (m, 4H) 3.60-3.71 (m, 4H) 4.15 (br s, 1H) 4.22 (br s, 1H) 4.30 (br s, 1H) 5.27 (s, 1H) 6.33 (d, J=3.91 Hz, 1H) 6.59 (br s, 1H) 6.90 (br dd, J=8.74, 4.22 Hz, 2H) 7.17 (br d, J=7.82 Hz, 3H) 7.37-7.43 (m, 2H) 7.61 (br d, J=10.15 Hz, 1H) 7.71 (t, J=7.89 Hz, 1H) 8.81 (s, 1H) 9.91 (br s, 1H) 10.26 (s, 1H) 12.43 (br s, 1H).

### Compound 28: N-(3-(6-(4-((4-(2-(4-(4-(2,4-dioxotetrahydropyrimidin-1(2H)-yl)phenoxy)piperidin-1-yl)ethyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

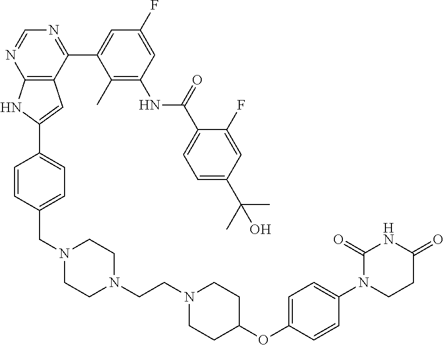 [SMILES: Cc1c(NC(=O)c2ccc(C(C)(C)O)cc2F)cc(F)cc1-c1ncnc2[nH]c(-c3ccc(CN4CCN(CCN5CCC(Oc6ccc(N7CCC(=O)NC7=O)cc6)CC5)CC4)cc3)cc12]

### Step 1: tert-Butyl 4-(2-oxoethyl)piperazine-1-carboxylate

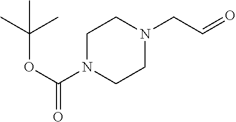 [SMILES: CC(C)(C)OC(=O)N1CCN(CC=O)CC1]

To a stirring solution of oxalyl chloride (380 μL, 4.25 mmol) in anhydrous DCM (10 mL) cooled down to −78° C. was added DMSO (538 μL, 7.58 mmol). The RM was stirred for 30 min, then 1-Boc-4-(2-hydroxyethyl)piperazine (CAS No. [77279-24-4]) (500 mg, 2.106 mmol) in DCM (10 mL) was added. The RM was stirred at the same temperature for 30 min, followed by the addition of TEA (2.4 mL, 17.22 mmol). The RM was then stirred for 1.5 h while allowing to reach RT, then quenched by sat. aq. NaHCO₃ solution and extracted with DCM (3×). The combined organic layers were dried over Na₂SO₄ and the solvent was removed under reduced pressure. The crude product was purified by flash chromatography on silica gel eluting with 0-30% EtOAc/MeOH 4/1) in EtOAc to afford the title compound as a pale yellow residue (371 mg). ¹H NMR (400 MHz, DMSO-d₆) δ 9.57 (d, J=1.5 Hz, 1H), 3.31 (dd, J=10.0, 5.0 Hz, 4H), 3.18 (d, J=1.5 Hz, 2H), 2.38 (t, J=5.1 Hz, 4H), 1.38 (s, 9H).

### Step 2: tert-Butyl 4-(2-(4-(4-(2,4-dioxotetrahydropyrimidin-1(2H)-yl)phenoxy)piperidin-1-yl)ethyl)piperazine-1-carboxylate

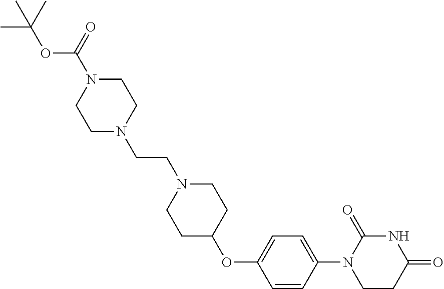 [SMILES: CC(C)(C)OC(=O)N1CCN(CCN2CCC(Oc3ccc(N4CCC(=O)NC4=O)cc3)CC2)CC1]

At RT, tert-butyl 4-(2-oxoethyl)piperazine-1-carboxylate (73 mg, 0.304 mmol), TEA (100 μL, 0.717 mmol) and 1-(4-(piperidin-4-yloxy)phenyl)dihydropyrimidine-2,4(1H,3H)-dione (ILB-36, 110 mg, 0.254 mmol) were dissolved in MeOH (2 mL). Then, ZnCl₂ 0.7 M in THF (400 μL, 0.280 mmol) was added and the RM was stirred overnight at RT under argon. Then, NaBH₃CN (19 mg, 0.302 mmol) was added and the RM was stirred overnight at RT. More tert-butyl 4-(2-oxoethyl)piperazine-1-carboxylate (73 mg, 0.304 mmol) was added and the RM was stirred for 3 days at RT under argon, then evaporated. The crude product was purified by reverse phase chromatography on a Redisep® C18 column eluting with ACN in an aq. solution of TFA (0.1%) (from 2 to 90%) to afford, after freeze drying, the title as a white powder TFA salt (130 mg). Method LCMS1: Rt=0.66 min; [M+H]⁺502.3.

### Step 3: 1-(4-((1-(2-(piperazin-1-yl)ethyl)piperidin-4-yl)oxy)phenyl)dihydropyrimidine-2,4(1H,3H)-dione

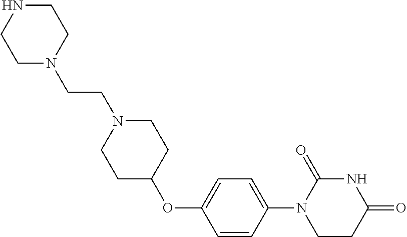 [SMILES: O=C1CCN(c2ccc(OC3CCN(CCN4CCNCC4)CC3)cc2)C(=O)N1]

A solution of tert-butyl 4-(2-(4-(4-(2,4-dioxotetrahydropyrimidin-1(2H)-yl)phenoxy)piperidin-1-yl)ethyl)piperazine-1-carboxylate (130 mg, 0.192 mmol) and TFA (250 μL, 3.24 mmol) in DCM (2 mL) was stirred for 2 h at RT. The RM was concentrated to dryness, the crude product was purified by reverse phase chromatography on a Redisep® C18 column eluting with ACN in an aq. solution of TFA (0.1%) (from 2 to 100%) to afford, after freeze drying, the title compound as a white solid TFA salt (41 mg). Method LCMS2: Rt=0.65 min; [M+H]⁺=402.3.

### Step 4: N-(3-(6-(4-((4-(2-(4-(4-(2,4-dioxotetrahydropyrimidin-1(2H)-yl)phenoxy)piperidin-1-yl)ethyl)piperazin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

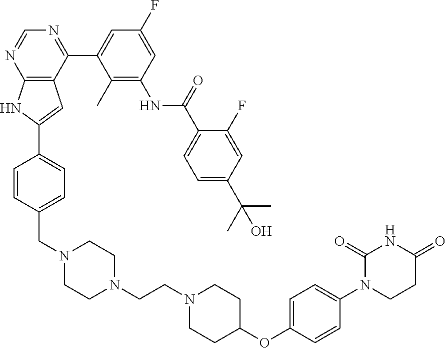 [SMILES: Cc1c(NC(=O)c2ccc(C(C)(C)O)cc2F)cc(F)cc1-c1ncnc2[nH]c(-c3ccc(CN4CCN(CCN5CCC(Oc6ccc(N7CCC(=O)NC7=O)cc6)CC5)CC4)cc3)cc12]

At RT, 1-(4-((1-(2-(piperazin-1-yl)ethyl)piperidin-4-yl)oxy)phenyl)dihydropyrimidine-2,4(1H,3H)-dione (41 mg, 0.040 mmol), TEA (50 μL, 0.359 mmol) and 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 29 mg, 0.055 mmol) were dissolved in MeOH (500 μL). Then, ZnCl₂ 0.7 M in THF (100 μL, 0.070 mmol) was added and the RM was stirred overnight at RT under argon. Then NaBH₃CN (5 mg, 0.080 mmol) was added and the RM was stirred overnight at RT, then evaporated. The crude product was purified by reverse phase chromatography on a Redisep® C18 column eluting with ACN in an aq. solution of NH₄HCO₃ (0.1%) (from 2 to 90%) to afford, after partial evaporation, a cloudy white suspension. The suspension was cooled down to 0° C., filtered to afford a non-pure material, that was then purified by SFC using Method XU (column: Princeton PPU 250×30 mm, 100 Å, 5 μM) eluting with 35-50% CO₂ in MeOH to afford the title compound (10.3 mg). Method LCMS1: Rt=0.70 min; [M+H]⁺=912.6. Method LCMS5: Rt=3.14 min; [M+H]⁺=912.6.

### Compound 29: N-(3-(6-(4-(((2-(4-(4-(2,4-dioxotetrahydropyrimidin-1(2H)-yl)phenoxy)piperidin-1-yl)ethyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

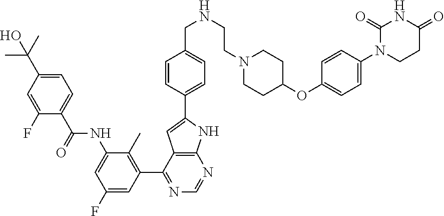 [SMILES: Cc1c(NC(=O)c2ccc(C(C)(C)O)cc2F)cc(F)cc1-c1ncnc2[nH]c(-c3ccc(CNCCN4CCC(Oc5ccc(N6CCC(=O)NC6=O)cc5)CC4)cc3)cc12]

To a yellow-green mixture of 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 60 mg, 0.114 mmol), 1-(4-((1-(2-aminoethyl)piperidin-4-yl)oxy)phenyl)dihydropyrimidine-2,4(1H,3H)-dione (ILB-68, 78 mg, 0.125 mmol) and TEA (35 μL, 0.251 mmol) in dry MeOH (2.3 mL) flushed with N₂ was added ZnCl₂ 0.5 M in THF (251 μL, 0.125 mmol). The resulting RM was flushed with N₂ and stirred at RT for 4 h. Then, NaBH₃CN (11.3 mg, 0.171 mmol) was added and it was stirred at RT for 18 h. The RM was diluted with ACN, adsorbed on Isolute®, concentrated until dryness, dried under HV pump and purified by reverse phase chromatography on a Redisep® C18 column eluting with ACN in an aq. solution of TFA (0.10%) to afford, after filtration of the fractions containing the pure target compound through PL-HCO₃ MP SPE cartridges and freeze drying, the title compound as an off-white solid (76 mg). Method LCMS1): Rt=0.75 min; [M+H]⁺=843.5. Method LCMS5): Rt=3.33 min; [M+H]⁺=843.4. 1H NMR (400 MHz, DMSO-d₆): 1.45 (s, 6H) 1.60 (q, J=9.66 Hz, 2H) 1.87-1.96 (m, 2H) 2.15-2.23 (m, 5H) 2.40-2.44 (m, 2H) 2.56-2.61 (m, 2H) 2.64-2.70 (m, 4H) 3.65-3.77 (m, 4H) 4.28-4.41 (m, 1H) 5.29 (s, 1H) 6.83 (s, 1H) 6.94 (d, J=8.93 Hz, 2H) 7.17-7.26 (m, 3H) 7.39-7.45 (m, 4H) 7.66 (br d, J=9.90 Hz, 1H) 7.70-7.76 (m, 1H) 7.94 (d, J=8.19 Hz, 2H) 8.84 (s, 1H) 9.93 (s, 1H) 10.28 (s, 1H) 12.74 (br s, 1H).

### Compound 30: N-(3-(6-(4-((4-((1-(2-(4-(2,4-dioxotetrahydropyrimidin-1(2H)-yl)-3-methylphenoxy)ethyl)piperidin-4-yl)oxy)piperidin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

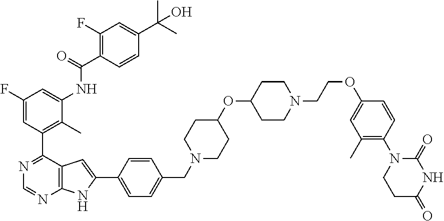 [SMILES: Cc1cc(OCCN2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O]

### Step 1: 2-(4-(2,4-dioxotetrahydropyrimidin-1(2H)-yl)-3-methylphenoxy)acetaldehyde, 2-(4-(2,4-dioxotetrahydropyrimidin-1(2H)-yl)-3-methylphenoxy)-1-hydroxyethanesulfonic acid

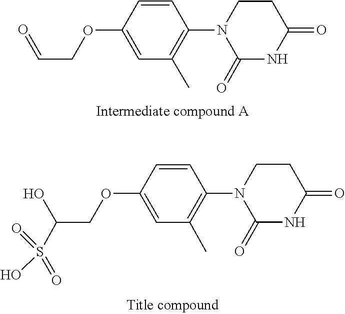 [SMILES: Cc1cc(OCC=O)ccc1N1CCC(=O)NC1=O ; Cc1cc(OCC(O)S(=O)(=O)O)ccc1N1CCC(=O)NC1=O]

Formation of Intermediate compound A: To a mixture of 1-(4-(2,2-diethoxyethoxy)-2-methylphenyl)dihydropyrimidine-2,4(1H,3H)-dione (0.49 g, 1.457 mmol) in THF (3.6 mL) was added HCl 2N (3.64 mL, 7.28 mmol). The RM was heated at 80° C. for 3 h. The yellow solution was immersed in an ice-bath. No precipitation after 0.5 h. Therefore the THF was evaporated off. The remaining aq. layer was freeze dried overnight. The crude material was evaporated and absorbed on silica gel and purified by flash chromatography on a silica flash column 12 g eluting with DCM/MeOH to afford the intermediate compound A (0.52 g).

Formation of title compound: Intermediate compound A was dissolved in EtOH (2 mL, Ratio: 12.50)/Water (0.16 mL, Ratio: 1.0) then treated with sodium metabisulfite (0.388 g, 2.039 mmol)—theoretical 0.7 eq/1 eq aldehyde—and heated at 80° C. for 1.5 h. The suspension was cooled to RT, diluted with 2 mL EtOH then the precipitate was filtered off, washed with EtOH and dried overnight at 50° C. in vacuo to afford the title compound (51.6 mg). Method LCMS1: Rt=0.33 min; [M−H]⁺=343.1.

### Step 2: N-(3-(6-(4-((4-((1-(2-(4-(2,4-dioxotetrahydropyrimidin-1(2H)-yl)-3-methylphenoxy)ethyl)piperidin-4-yl)oxy)piperidin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

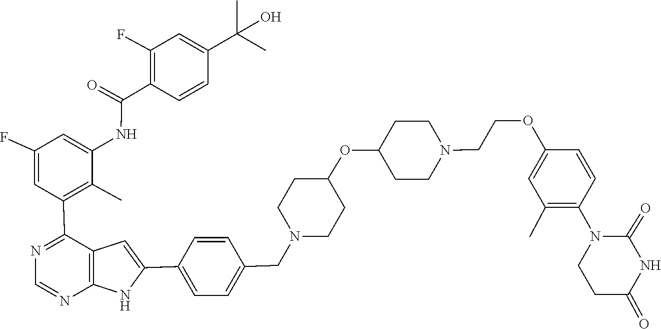 [SMILES: Cc1cc(OCCN2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O]

A solution of 2-fluoro-N-(5-fluoro-2-methyl-3-(6-(4-((4-(piperidin-4-yloxy)piperidin-1-yl)methyl)phenyl)-7H-pyrrolo[2,3-d]pyrimidin-4-yl)phenyl)-4-(2-hydroxypropan-2-yl)benzamide HCl salt (compound 8 step 2 in PCT/IB2019/052346 30 mg, 0.043 mmol) in MeOH (Volume: 1.5 mL) was treated with NaOAc (17.71 mg, 0.216 mmol) and stirred at room temperature for 15 minutes. Afterwards 2-(4-(2,4-dioxotetrahydropyrimidin-1(2H)-yl)-3-methylphenoxy)-1-hydroxyethanesulfonic acid (20.62 mg, 0.056 mmol) and picoline borane complex (5.43 mg, 0.043 mmol) was added. The RM was stirred overnight at RT. The slurry RM was diluted with MeOH then filtered through a disposal syringe filter. The filtrate was ready to inject in the prep. RP-HPLC with method XN. The crude compound was purified by prep. HPLC Method XN (reversed phase) on a Reprosil 100 C18 column eluting with ACN/aq. solution of TFA 0.1%. F54 was worked up (addition of NaHCO₃, extraction with AcOEt). The residue was dissolved in water/some drops of ACN and t-BuOH and freeze dried overnight to afford the title compound (3 mg) as a white solid. Method LCMS5: Rt=3.12 min; [M+H]⁺=941.3. ¹H NMR (600 MHz, DMSO-d₆) δ 12.77 (s, 1H), 10.29 (s, 1H), 9.96 (s, 1H), 8.85 (s, 1H), 7.94 (d, J=7.8 Hz, 2H), 7.73 (t, J=7.8 Hz, 1H), 7.66 (d, J=8.4 Hz, 1H), 7.45-7.36 (m, 4H), 7.24 (m, 1H), 7.14 (d, J=8.6 Hz, 1H), 6.88-6.75 (m, 3H), 5.31 (s, 1H), 4.03 (s, 2H), 3.69 (m, 1H), 3.51-3.43 (m, 4H), 3.38 (s, 2H), 2.87-2.70 (m, 4H), 2.70-2.62 (m, 6H), 2.15 (m, 9H), 1.77 (s, 3H), 1.45 (m, 10H).

### Compound 31: N-(3-(6-(4-((9-(2-(3-chloro-4-(2,4-dioxotetrahydropyrimidin-1(2H)-yl)phenoxy)ethyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

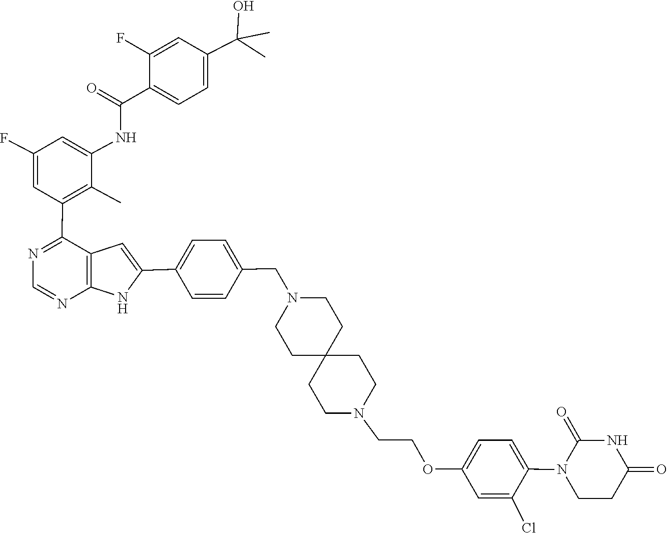 [SMILES: Cc1c(NC(=O)c2ccc(C(C)(C)O)cc2F)cc(F)cc1-c1ncnc2[nH]c(-c3ccc(CN4CCC5(CCN(CCOc6ccc(N7CCC(=O)NC7=O)c(Cl)c6)CC5)CC4)cc3)cc12]

### Step 1: 2-(3-chloro-4-(2,4-dioxotetrahydropyrimidin-1(2H)-yl)phenoxy)acetaldehyde, 2-(3-chloro-4-(2,4-dioxotetrahydropyrimidin-1(2H)-yl)phenoxy)-1-hydroxyethanesulfonic acid

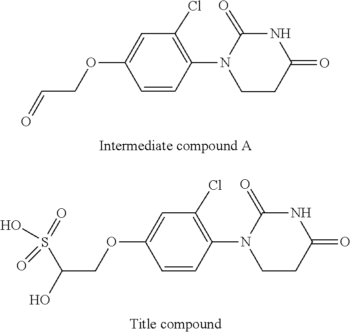 [SMILES: O=CCOc1ccc(N2CCC(=O)NC2=O)c(Cl)c1 ; O=C1CCN(c2ccc(OCC(O)S(=O)(=O)O)cc2Cl)C(=O)N1]

### Formation of Intermediate Compound A:

A mixture of 1-(2-chloro-4-(2,2-diethoxyethoxy)phenyl)dihydropyrimidine-2,4(1H,3H)-dione (0.49 g, 1.373 mmol) and HCl 2M aq. (0.034 mL, 0.069 mmol) in ACN (5 mL, Ratio: 31.3) was stirred at RT. After stirring overnight at RT, was added again HCl 2 M aq. (0.103 mL, 0.206 mmol). After stirring at RT, the RM was diluted with DCM and washed with water. The organic layer was separated, dried over sodium sulfate, filtered, and concentrated in vacuo to obtain 0.34 g of the crude Intermediate compound A as a brown resin. The crude material was purified by flash chromatography on a silica flash column 4 g eluting with DCM/MeOH to afford the Intermediate compound A (0.24 g).

### Formation of Title Compound:

Intermediate Compound A was dissolved in EtOH (2 mL, Ratio: 12.5)/Water (0.16 mL, Ratio: 1.0) then treated with sodium metabisulfite (0.091 g, 0.481 mmol)—theoretical 0.7 eq/1 eq aldehyde—and heated at 80° C. for 1.5 h. The suspension was cooled to RT, diluted with 2 mL EtOH, then the precipitate was filtered off, washed with EtOH, and dried overnight at 50° C. in vacuo to obtain the title compound as the sodium salt (265 mg). Method LCMS1: Rt=0.35 min; [M−H]⁺=363.0.

### Step 2: N-(3-(6-(4-((9-(2-(3-chloro-4-(2,4-dioxotetrahydropyrimidin-1(2H)-yl)phenoxy)ethyl)-3,9-diazaspiro[5.5]undecan-3-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

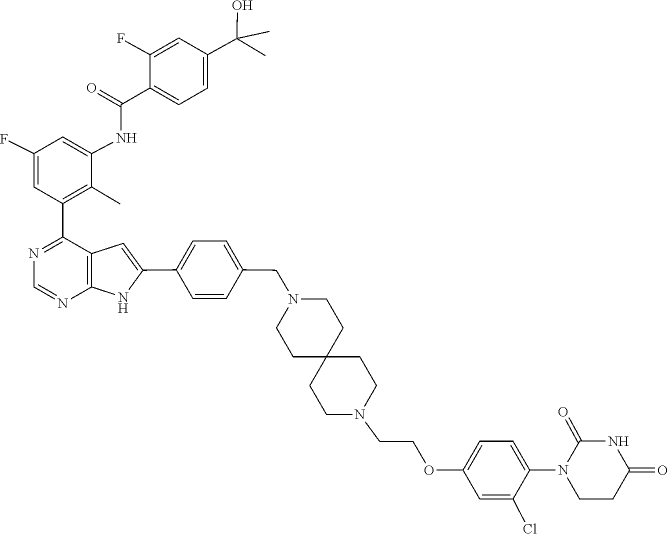 [SMILES: Cc1c(NC(=O)c2ccc(C(C)(C)O)cc2F)cc(F)cc1-c1ncnc2[nH]c(-c3ccc(CN4CCC5(CCN(CCOc6ccc(N7CCC(=O)NC7=O)c(Cl)c6)CC5)CC4)cc3)cc12]

A solution of N-(3-(6-(4-(3,9-diazaspiro[5.5]undecan-3-ylmethyl)phenyl)-7H-pyrrolo[2,3-d]pyrimidin-4-yl)-5-fluoro-2-methylphenyl)-2-fluoro-4-(2-hydroxypropan-2-yl)benzamide (intermediate 6 in PCT/IB2019/052346 60 mg, 0.056 mmol) in MeOH (1.5 mL) was treated with NaOAc (22.96 mg, 0.280 mmol) and stirred at RT for 15 minutes. Afterwards 2-(3-chloro-4-(2,4-dioxotetrahydropyrimidin-1(2H)-yl)phenoxy)-1-hydroxyethanesulfonic acid (28.2 mg, 0.073 mmol) and picoline borane complex (7.04 mg, 0.056 mmol) was added. The RM was stirred overnight at RT. The slurry RM was diluted with MeOH then filtered through a disposal syringe filter. The filtrate was ready to inject in the prep. RP-HPLC. The crude compound was purified by prep. HPLC (reversed phase) on a Reprosil® 100 C18 column eluting with ACN/aq. solution of TFA 0.1% using the method XN. Title compound was worked up (addition of NaHCO3, extraction with AcOEt). The residue was dissolved in water and some drops of ACN and t-BuOH and freeze dried overnight to afford the title compound (16.9 mg). Method LCMS5: Rt=2.97 min; [M+H]⁺=932.2. ¹H NMR (400 MHz, DMSO-d₆) δ 12.76 (s, 1H), 10.41 (s, 1H), 9.94 (d, J=2.5 Hz, 1H), 8.85 (s, 1H), 7.93 (d, J=7.9 Hz, 2H), 7.83-7.59 (m, 2H), 7.51-7.32 (m, 5H), 7.31-7.10 (m, 2H), 6.97 (dd, J=8.8, 2.8 Hz, 1H), 6.82 (s, 1H), 5.30 (s, 1H), 4.18 (s, 1H), 4.10 (m, 2H), 3.69-3.60 (m, 1H), 3.59-3.44 (m, 3H), 2.72 (t, J=6.0 Hz, 2H), 2.43 (m, 6H), 2.37-2.26 (m, 5H), 2.17 (s, 3H), 1.43 (m, J=14.2 Hz, 14H).

### Compound 32: (3R,4S)—N-(3-(6-(4-((4-(2-(4-(2,4-Dioxotetrahydropyrimidin-1(2H)-yl)phenoxy)ethyl)piperazin-1-yl)methyl)phenyl)-7H-pyrrolo[2,3-d]pyrimidin-4-yl)-5-fluoro-2-methylphenyl)-3-hydroxy-4-isobutylpyrrolidine-1-carboxamide

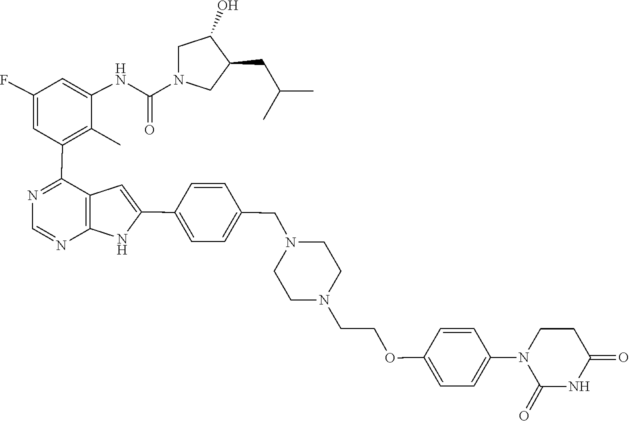 [SMILES: Cc1c(NC(=O)N2C[C@H](CC(C)C)[C@@H](O)C2)cc(F)cc1-c1ncnc2[nH]c(-c3ccc(CN4CCN(CCOc5ccc(N6CCC(=O)NC6=O)cc5)CC4)cc3)cc12]

### Step 1: (3R,4S)—N-(5-Fluoro-3-(6-(4-formylphenyl)-7H-pyrrolo[2,3-d]pyrimidin-4-yl)-2-methylphenyl)-3-hydroxy-4-isobutylpyrrolidine-1-carboxamide

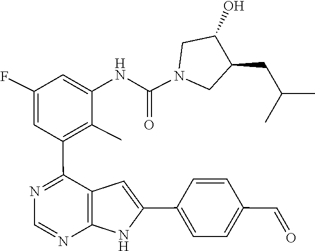 [SMILES: Cc1c(NC(=O)N2C[C@H](CC(C)C)[C@@H](O)C2)cc(F)cc1-c1ncnc2[nH]c(-c3ccc(C=O)cc3)cc12]

In a round-bottomed flask was added 4-(4-chloro-7H-pyrrolo[2,3-d]pyrimidin-6-yl)benzaldehyde (intermediate 3a in PCT/IB2019/052392, 150 mg, 0.582 mmol), (3R,4S)—N-(5-fluoro-2-methyl-3-(4,4,5,5-tetramethyl-1,3,2-dioxaborolan-2-yl)phenyl)-3-hydroxy-4-isobutylpyrrolidine-1-carboxamide (intermediate 7 in PCT/IB2019/052392, 269 mg, 0.640 mmol), and K₂CO₃ (201 mg, 1.455 mmol), PdCl2(dppf) (42.6 mg, 0.058 mmol) was added. The reaction mixture was diluted with dioxane (3 mL, Ratio: 1.0) and with H₂O (3 mL, Ratio: 1.0). The RM was heated at 100° C. for 1.5 h. The RM was absorbed on silica and purified by flash chromatography on a 12 g column eluting DCM/MeOH to afford the title compound (90 mg). Method LCMS1: Rt=0.95 min; [M+H]⁺=516.2.

### Step 2: (3R,4S)—N-(3-(6-(4-((4-(2-(4-(2,4-dioxotetrahydropyrimidin-1(2H)-yl)phenoxy)ethyl)piperazin-1-yl)methyl)phenyl)-7H-pyrrolo[2,3-d]pyrimidin-4-yl)-5-fluoro-2-methylphenyl)-3-hydroxy-4-isobutylpyrrolidine-1-carboxamide

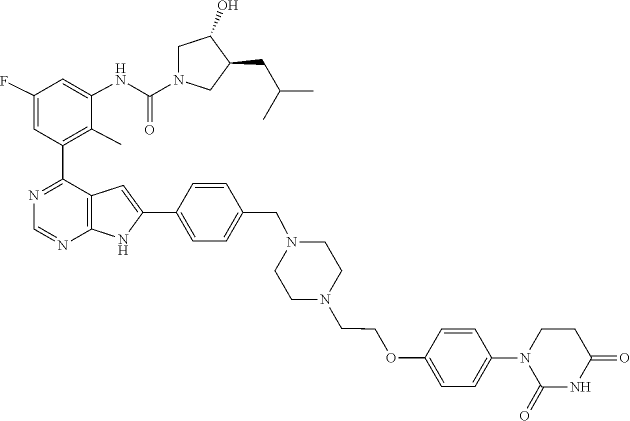 [SMILES: Cc1c(NC(=O)N2C[C@H](CC(C)C)[C@@H](O)C2)cc(F)cc1-c1ncnc2[nH]c(-c3ccc(CN4CCN(CCOc5ccc(N6CCC(=O)NC6=O)cc5)CC4)cc3)cc12]

In a round-bottomed flask was added (3R,4S)—N-(5-fluoro-3-(6-(4-formylphenyl)-7H-pyrrolo[2,3-d]pyrimidin-4-yl)-2-methylphenyl)-3-hydroxy-4-isobutylpyrrolidine-1-carboxamide (103 mg, 0.200 mmol), 1-(4-(2-(piperazin-1-yl)ethoxy)phenyl)dihydropyrimidine-2,4(1H,3H)-dione (ILB-35, 131 mg, 0.240 mmol), and TEA (0.056 mL, 0.400 mmol) in MeOH (3 mL) to give a colorless solution. ZnCl₂ in solution in THF (0.420 mL, 0.210 mmol) was added. The RM was stirred 3 h under argon. After this time, NaBH₃CN (25.1 mg, 0.400 mmol) was added and the RM was stirred at RT overnight. The solution was diluted with CH₂Cl₂ and washed with Water and brine. The organic layer (suspension) was evaporated und purified by reverse phase. The crude compound was absorbed on Isolute and purified by reverse phase chromatography on a Redisep® C18 column eluting with ACN/aq. solution of TFA (0.1%). The most pure fractions were further purified by reverse phase prep HPLC using method XS (XBridge Prep C18 OBD 5 μm 100×30 mm+0.1% TFA 13-43% ACN in 15 min at 30 mL/min). Bond Elut SCX 2 mg/12 mL from Agilent was used to remove the TFA. The column was washed 2 times with 10 mL of MeOH and then the title compound was recovered from the cartridge by washing with ammonia in MeOH 7 N. The filtrate was evaporated to dryness to afford the title compound (33.6 mg). Method LCMS1: Rt=0.84 min; [M−H]⁺=816.5. Method LCMS5: Rt=3.49 min; [M−H]⁺=816.5. ¹H NMR (400 MHz, DMSO-d6) S 12.73 (s, 1H), 10.29 (s, 1H), 8.83 (s, 1H), 7.93 (d, J=7.8 Hz, 2H), 7.67 (s, 1H), 7.57-7.30 (m, 3H), 7.21 (d, J=8.8 Hz, 2H), 7.06 (dd, J=8.9, 2.7 Hz, 1H), 6.94 (d, J=8.8 Hz, 2H), 6.77 (s, 1H), 5.12 (d, J=4.5 Hz, 1H), 4.06 (s, 2H), 3.94-3.82 (m, 1H), 3.75-3.59 (m, 5H), 3.50 (s, 2H), 3.30 (m, 1H), 3.19 (m, 1H), 3.14-3.01 (m, 1H), 2.68 (t, J=6.6 Hz, 5H), 2.37 (m, 4H), 2.09 (m, 4H), 1.62 (m, 1H), 1.35 (m, 1H), 1.12 (m, 1H), 0.90 (m, 7H).

### Compound 33: N-(3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)-2-fluoro-4-(2-hydroxypropan-2-yl)benzamide

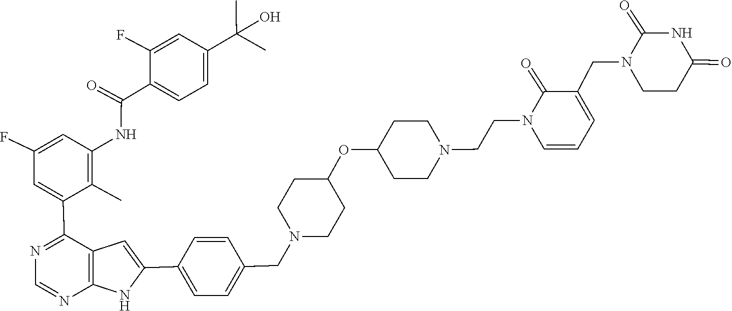 [SMILES: Cc1c(NC(=O)c2ccc(C(C)(C)O)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

To a solution of 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 56 mg, 0.13 mmol) and 1-((2-oxo-1-(2-(4-(piperidin-4-yloxy)piperidin-1-yl)ethyl)-1,2-dihydropyridin-3-yl)methyl)dihydropyrimidine-2,4(1H,3H)-dione (ILB-6, 89 mg, 0.169 mmol) in 1 mL DMSO was added ZnCl₂ (0.2 6 mL 1M solution in THF, 0.26 mmol). The reaction mixture was stirred at 10° C. for 2 h. To the mixture was added NaBH₃CN (26 mg, 0.416 mmol) and the mixture was stirred at RT for 18 h. 2 mL MeOH was added, the mixture was filtered and purified by preparative HPLC (Xbridge C18, 21.2×250 mm, 10 μm), eluting with 0.01M ammonium hydrogen carbonate buffer in H₂O/ACN yielding the target compound as light yellow solid (11 mg). Method LCMS XG: Rt=1.99 min; (M+H)+=942. ¹H NMR (500 MHz, DMSO-d6) δ 12.77 (br s, 1H), 10.16 (s, 1H), 9.95 (d, J=2.0 Hz, 1H), 8.85 (s, 1H), 7.94 (d, J=8.5 Hz, 2H), 7.75-7.72 (m, 1H), 7.67-7.65 (m, 1H), 7.58-7.56 (m, 1H), 7.44-7.37 (m, 4H), 7.27-7.23 (m, 2H), 6.83 (s, 1H), 6.21-6.18 (m, 1H), 5.31 (s, 1H), 4.26 (s, 2H), 3.99-3.97 (m, 2H), 3.47 (s, 2H), 3.42-3.37 (m, 4H), 2.73-2.64 (m, 4H), 2.58-2.55 (m, 2H), 2.54-2.50 (m, 2H), 2.18 (s, 3H), 2.12-2.05 (m, 4H), 1.74-1.71 (m, 4H), 1.45 (s, 6H), 1.42-1.32 (m, 4H).

### Compound 34: N-(3-(6-(4-(((4-(2-(3-((2,4-dioxotetrahydropyrimidin-1(2H)-yl)methyl)-2-oxopyridin-1(2H)-yl)ethoxy)butyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

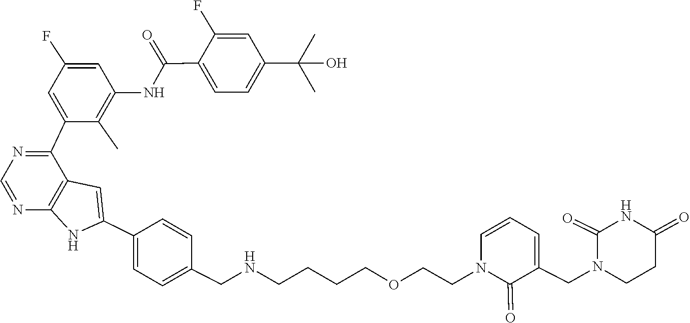 [SMILES: Cc1c(NC(=O)c2ccc(C(C)(C)O)cc2F)cc(F)cc1-c1ncnc2[nH]c(-c3ccc(CNCCCCOCCn4cccc(CN5CCC(=O)NC5=O)c4=O)cc3)cc12]

### Step 1: N-(3-(6-(4-(aminomethyl)phenyl)-7H-pyrrolo[2,3-d]pyrimidin-4-yl)-5-fluoro-2-methylphenyl)-2-fluoro-4-(2-hydroxypropan-2-yl)benzamide

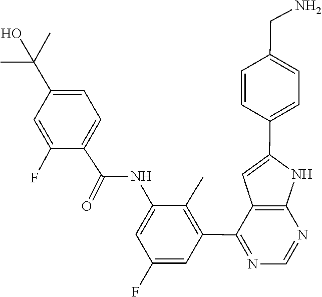 [SMILES: Cc1c(NC(=O)c2ccc(C(C)(C)O)cc2F)cc(F)cc1-c1ncnc2[nH]c(-c3ccc(CN)cc3)cc12]

A yellow mixture of 2-fluoro-N-(5-fluoro-3-(6-(4-formylphenyl)-7H-pyrrolo[2,3-d]pyrimidin-4-yl)-2-methylphenyl)-4-(2-hydroxypropan-2-yl)benzamide (intermediate 3 in PCT/IB2019/052346 200 mg, 0.380 mmol), NH₃ 7 N in MeOH (5 mL, 35 mmol) and Raney-Nickel (Ra—Ni (EtOH) Degussa B113W) (100 mg) was shaken at RT under 3.5 bar of H₂ in a glass autoclave/Hastelloy®. After 16.5 h at RT, the RM was diluted with more NH₃ 7 N in MeOH (25 mL, 175 mmol). More Raney-Nickel (200 mg) was added and the RM was shaken under 3.5 bar of H₂ at RT for 2 additional nights. The RM was filtered through a pad of Celite® filter aid, rinsed with ACN and MeOH, to afford a grey-green solid. The solid was diluted in ACN/MeOH, adsorbed on Isolute®, concentrated until dryness, dried under HV pump and purified by reverse phase chromatography on a Redisep® C18 column eluting with ACN in an aq. solution of TFA (0.1%) (from 10 to 100%) to afford, after freeze drying, the title compound as a yellow solid TFA salt (147 mg). Method LCMS1: Rt=0.72 min; [M+H]⁺=528.3.

### Step 2: N-(3-(6-(4-(((4-(2-(3-((2,4-dioxotetrahydropyrimidin-1(2H)-yl)methyl)-2-oxopyridin-1(2H)-yl)ethoxy)butyl)amino)methyl)phenyl)-7H-pyrrolo[2,3-d]pyrimidin-4-yl)-5-fluoro-2-methylphenyl)-2-fluoro-4-(2-hydroxypropan-2-yl)benzamide

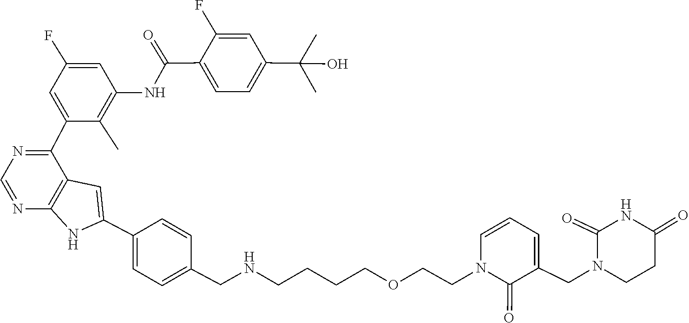 [SMILES: Cc1c(NC(=O)c2ccc(C(C)(C)O)cc2F)cc(F)cc1-c1ncnc2[nH]c(-c3ccc(CNCCCCOCCn4cccc(CN5CCC(=O)NC5=O)c4=O)cc3)cc12]

To a mixture of N-(3-(6-(4-(aminomethyl)phenyl)-7H-pyrrolo[2,3-d]pyrimidin-4-yl)-5-fluoro-2-methylphenyl)-2-fluoro-4-(2-hydroxypropan-2-yl)benzamide trifluoroacetate salt (110 mg, 0.219 mmol), K₂CO₃ (43 mg, 0.3135 mmol) and 4-(2-(3-((2,4-dioxotetrahydropyrimidin-1(2H)-yl)methyl)-2-oxopyridin-1(2H)-yl)ethoxy)butanal (ILB-5, 70 mg, 0.149 mmol) in DMSO (5 mL) was added ZnCl₂ (0.223 mL, 0.223 mmol). The mixture was stirred at RT for 30 min, then NaBH₃CN (56 mg, 0.892 mmol) was slowly added. The reaction mixture was stirred at room temperature for 4 h. The mixture filtered and directly purified by reverse-phase chromatography Method PB to obtain the title compound as a white solid. (15 mg). LC-MS Method H: Rt=1.93 min, (M+H)⁺=848.3. ¹H NMR (500 MHz, DMSO-d₆) δ 12.74 (s, 1H), 10.16 (s, 1H), 9.96 (s, 1H), 8.85 (s, 1H), 7.93 (d, J=8.2 Hz, 2H), 7.73 (t, J=7.9 Hz, 1H), 7.66 (d, J=9.3 Hz, 1H), 7.54 (d, J=5.0 Hz, 1H), 7.42 (dd, J=13.8, 7.4 Hz, 4H), 7.31-7.20 (m, 2H), 6.83 (s, 1H), 6.19 (t, J=6.8 Hz, 1H), 5.31 (s, 1H), 4.26 (s, 2H), 4.05 (t, J=5.3 Hz, 2H), 3.69 (s, 2H), 3.59 (t, J=5.4 Hz, 2H), 3.40 (t, J=6.8 Hz, 2H), 3.36-3.30 (m, 3H), 2.56 (t, J=6.8 Hz, 2H), 2.44 (t, J=6.9 Hz, 2H), 2.18 (s, 3H), 1.56-1.34 (m, 10H).

### Compound 35: (3R,4S)—N-(3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)-3-hydroxy-4-isobutylpyrrolidine-1-carboxamide

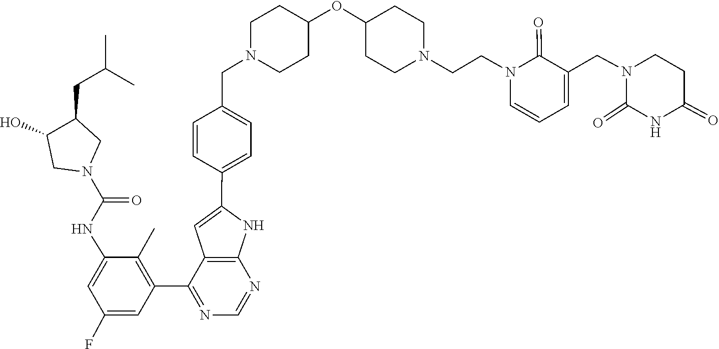 [SMILES: Cc1c(NC(=O)N2C[C@H](CC(C)C)[C@@H](O)C2)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

### Step 1: (3R,4S)—N-(5-fluoro-3-(6-(4-formylphenyl)-7H-pyrrolo[2,3-d]pyrimidin-4-yl)-2-methylphenyl)-3-hydroxy-4-isobutylpyrrolidine-1-carboxamide

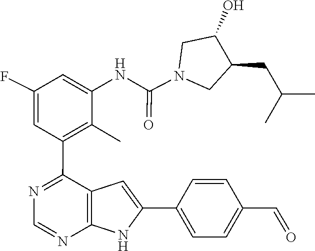 [SMILES: Cc1c(NC(=O)N2C[C@H](CC(C)C)[C@@H](O)C2)cc(F)cc1-c1ncnc2[nH]c(-c3ccc(C=O)cc3)cc12]

In a round-bottomed flask was added 4-(4-chloro-7H-pyrrolo[2,3-d]pyrimidin-6-yl)benzaldehyde (intermediate 3a in PCT/IB2019/052392, 150 mg, 0.582 mmol), (3R,4S)—N-(5-fluoro-2-methyl-3-(4,4,5,5-tetramethyl-1,3,2-dioxaborolan-2-yl)phenyl)-3-hydroxy-4-isobutylpyrrolidine-1-carboxamide (intermediate 7 in PCT/IB2019/052392, 269 mg, 0.640 mmol), and K₂CO₃ (201 mg, 1.455 mmol), PdCl₂(dppf) (42.6 mg, 0.058 mmol) was added. The reaction mixture was diluted with dioxane (3 mL, Ratio: 1.0) and water (3 mL, Ratio: 1.0). The RM was heated at 100° C. for 1.5 h. The RM was absorbed on silica and purified by flash chromatography on a 12 g column eluting DCM/MeOH to afford the title compound (90 mg). Method LCMS1: Rt=0.95 min; [M+H]⁺=516.2.

### Step 2: tert-Butyl 4-((1-(4-(4-(5-fluoro-3-((3R,4S)-3-hydroxy-4-isobutylpyrrolidine-1-carboxamido)-2-methylphenyl)-7H-pyrrolo[2,3-d]pyrimidin-6-yl)benzyl)piperidin-4-yl)oxy)piperidine-1-carboxylate

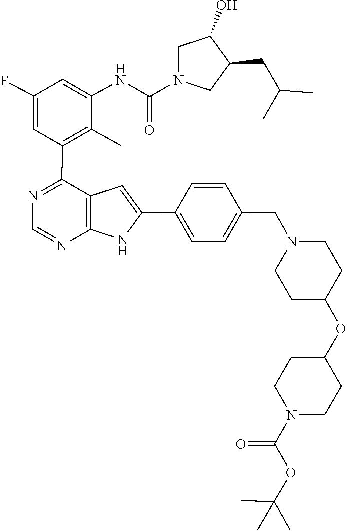 [SMILES: Cc1c(NC(=O)N2C[C@H](CC(C)C)[C@@H](O)C2)cc(F)cc1-c1ncnc2[nH]c(-c3ccc(CN4CCC(OC5CCN(C(=O)OC(C)(C)C)CC5)CC4)cc3)cc12]

(3R,4S)—N-(5-fluoro-3-(6-(4-formylphenyl)-7H-pyrrolo[2,3-d]pyrimidin-4-yl)-2-methylphenyl)-3-hydroxy-4-isobutylpyrrolidine-1-carboxamide (624 mg, 1.2 mmol) in DMSO/MeOH (10 mL) was added tert-butyl 4-(piperidin-4-yloxy)piperidine-1-carboxylate (CAS No. [845305-83-1], 344 mg, 1.2 mmol) and ZnCl₂ 1M in THF (1.8 mL, 1.8 mmol) and the mixture was stirred at RT for 2 h. NaBH₃CN (453 mg, 7.2 mmol) was added and the mixture was stirred at RT overnight. 10 mL of water was added to the mixture and stirred for 30 min and filtered to give crude product as the title compound. Method G: Rt=2.29 min; [M+H]⁺=784.

### Step 3: (3R,4S)—N-(5-fluoro-2-methyl-3-(6-(4-((4-(piperidin-4-yloxy)piperidin-1-yl)methyl)phenyl)-7H-pyrrolo[2,3-d]pyrimidin-4-yl)phenyl)-3-hydroxy-4-isobutylpyrrolidine-1-carboxamide

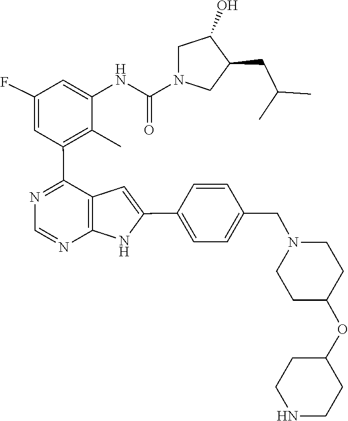 [SMILES: Cc1c(NC(=O)N2C[C@H](CC(C)C)[C@@H](O)C2)cc(F)cc1-c1ncnc2[nH]c(-c3ccc(CN4CCC(OC5CCNCC5)CC4)cc3)cc12]

To a solution of tert-butyl 4-((1-(4-(4-(5-fluoro-3-((3R,4S)-3-hydroxy-4-isobutylpyrrolidine-1-carboxamido)-2-methylphenyl)-7H-pyrrolo[2,3-d]pyrimidin-6-yl)benzyl)piperidin-4-yl)oxy)piperidine-1-carboxylate (1 g, 1.2 mmol) in THF (20 mL) and DCM (20 mL) was added HCl/dioxane (10 mL) at RT and the mixture was stirred at RT for 2 h. The RM was filtered and washed by DCM to afford the title compound (0.52 g). Method F: Rt=1.32 min; [M+H]⁺=684.

### Step 4: (3R,4S)—N-(3-(6-(4-((4-((1-(2-(3-((2,4-dioxotetrahydropyrimidin-1(2H)-yl)methyl)-2-oxopyridin-1(2H)-yl)ethyl)piperidin-4-yl)oxy)piperidin-1-yl)methyl)phenyl)-7H-pyrrolo[2,3-d]pyrimidin-4-yl)-5-fluoro-2-methylphenyl)-3-hydroxy-4-isobutylpyrrolidine-1-carboxamide

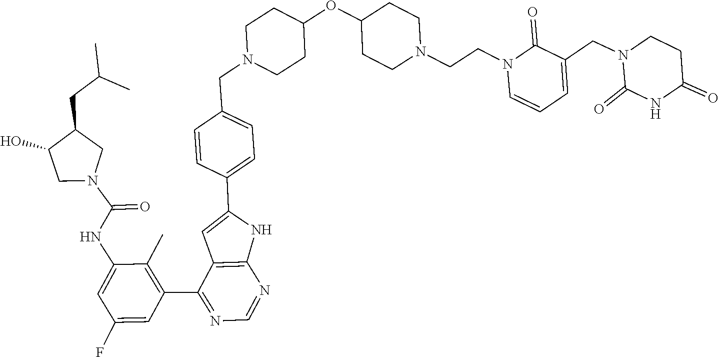 [SMILES: Cc1c(NC(=O)N2C[C@H](CC(C)C)[C@@H](O)C2)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

To the mixture of (3R,4S)—N-(5-fluoro-2-methyl-3-(6-(4-((4-(piperidin-4-yloxy)piperidin-1-yl)methyl)phenyl)-7H-pyrrolo[2,3-d]pyrimidin-4-yl)phenyl)-3-hydroxy-4-isobutylpyrrolidine-1-carboxamide (327 mg, 0.36 mmol) in DMSO (4 mL) was added K₂CO₃ (57 mg, 0.41 mmol), the solution was stirred 10 min at RT, and then 2-(3-((2,4-dioxotetrahydropyrimidin-1(2H)-yl)methyl)-2-oxopyridin-1(2H)-yl)acetaldehyde (91 mg, 0.35 mmol) and ZnCl₂ (0.9 mL, 1 M in THF) were added, after 0.5 h, NaBH₃CN (177 mg, 2.77 mmol) was added. The mixture was stirred at RT for 16 h. The mixture filtered to remove the solid and the filtrate was collected. The crude residue was purified by Prep-HPLC using method PB eluting with ACN and an aq. solution of NH₄HCO₃ aq. 10 mM to afford, after freeze drying, the title compound (60 mg). Method G: Rt=1.73 min; [M+H]⁺=932. ¹H NMR (500 MHz, DMSO-d₆) δ 12.72 (s, 1H), 10.14 (s, 1H), 8.83 (s, 1H), 7.92 (d, J=8.2 Hz, 2H), 7.67 (s, 1H), 7.59-9.54 (m, 1H), 7.50 (dd, J=10.8, 2.8 Hz, 1H), 7.37 (d, J=8.2 Hz, 2H), 7.27 (d, J=6.6 Hz, 1H), 7.06 (dd, J=8.9, 2.8 Hz, 1H), 6.76 (s, 1H), 6.19 (t, J=6.8 Hz, 1H), 5.11 (d, J=4.5 Hz, 1H), 4.26 (s, 2H), 3.98 (t, J=6.3 Hz, 2H), 3.91-3.84 (m, 1H), 3.69-3.61 (m, 2H), 3.47 (s, 2H), 3.41 (t, J=6.8 Hz, 4H), 3.22-3.15 (m, 1H), 3.12-3.04 (m, 1H), 2.72-2.62 (m, 4H), 2.59-2.51 (m, 4H), 2.16-2.00 (m, 8H), 1.79-1.69 (m, 4H), 1.67-1.56 (m, 1H), 1.46-1.30 (m, 5H), 1.18-1.07 (m, 1H), 0.90 (dd, J=10.7, 6.6 Hz, 6H).

### Example 7

### Biological Assays

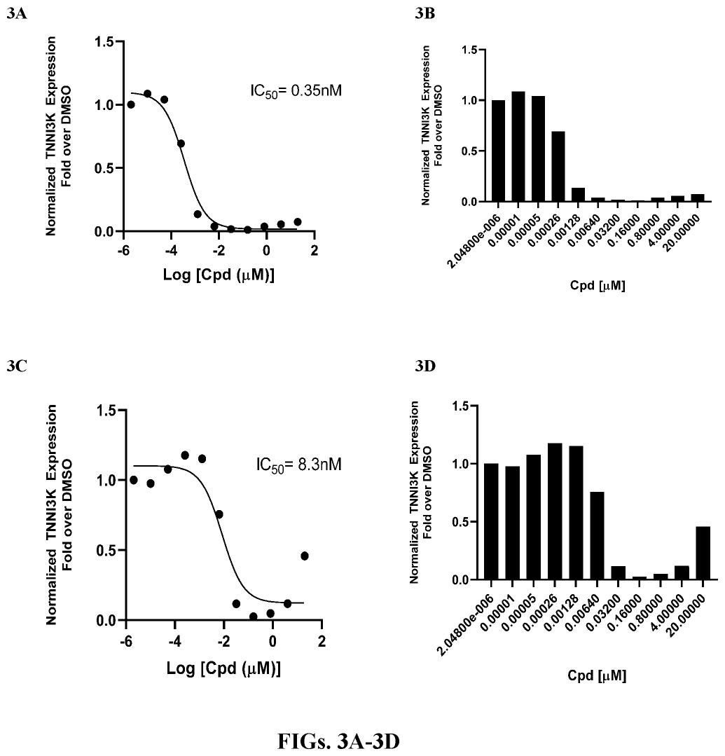

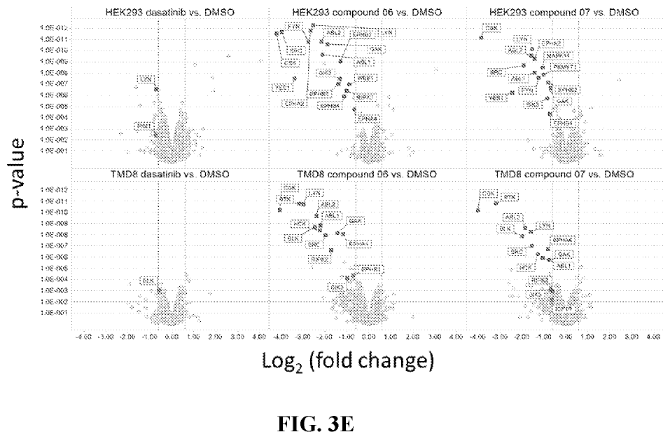

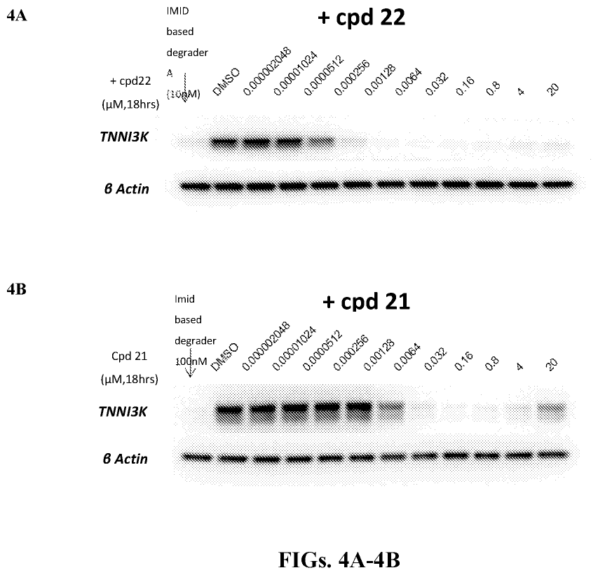

Compounds were tested in the following biochemical and cellular assays. The data obtained is shown in Tables 4, 5, and 6 and FIGS. 3A-3E and 4A-4B. The compounds disclosed herein were tested in the following biochemical assay to demonstrate CRBN interaction. The data obtained is shown in Table 4 and 5, and AC₅₀ refers to the concentration at which 50% of the reference probe Compound HH is displaced.

### CRBN Assay Format 1:

BodipyFL conjugated lenalidomide compound HH was used as a fluorescent probe. Enzymatic reactions were conducted in ‘assay buffer’, comprising 50 mM Tris/HCl at pH 7.4, 100 mM NaCl, 0.1% (w/v) Pluronic F-127 and 1 mM TCEP. Protein and substrate were diluted in assay buffer.

All protein and probe containing solutions were handled in ‘Maxymum Recovery’ tubes (Axygen Scientific Inc., Union City, USA). Compound, protein and the substrate solutions were transferred to 384-well plates (Black Microtiter 384 Plate, round well; Cat. No. 95040020 Thermo Electron Oy, Finland) by means of a CyBi-Well 96-channel pipettor (CyBio AG, Jena, Germany). For the plate measurements a PHERAstar reader (BMG Labtech, Offenburg, Germany) was used. The instrument was equipped with a specific optics module containing filters and dichoic mirrors for measuring the fluorescence polarization-type assay. With this module, the fluorescence of the Bodipy FL-based probe was excited at 485 nm and the emissions of the product were measured at 520 nm. The fluorescence in each well was excited by 10 flashes per measurement.

For the determination of AC₅₀ values, the assays were performed at room temperature in 384-well plates with a total assay volume of 10.1 μL per well. The test compound was dissolved in 90% (v/v) DMSO/water. For the assays, 100 nL of the 90% (v/v) DMSO/water solution or compound solution were added per well, followed by the addition of 5 μL protein solution (protein in 1× assay buffer).

The final assay concentration of the protein was nominally 100 nM. After 45 minutes of pre-incubation at room temperature, the competition for binding was started by the addition of 5 μL probe solution (probe dissolved in assay buffer). The final concentration of the probes in the assays was 5 nM. After the addition of the probe solution, the final DMSO concentration in the assay was 0.9% (v/v). The effect of the probe on the pre-established compound-protein complex equilibrium was determined after 45 minutes (t=45 min). The AC₅₀ value was calculated from the plot of percentage of protein saturation versus the test compound concentration by a logistics fit according to: y=A²+(A¹−A²)/(1+(x/AC₅₀)^{p}), where y is the %-saturation value at the test compound concentration, x. A¹ is the lowest saturation value, i.e., 0%, and A² the maximum saturation value, i.e. 100%. The exponent, p, is the Hill coefficient.

### CRBN Assay Format 2a:

BodipyFL conjugated lenalidomide compound HH was used as a fluorescent probe. Enzymatic reactions were conducted in ‘assay buffer’, comprising 50 mM Tris/HCl at pH 7.4, 100 mM NaCl, 0.1% (w/v) Pluronic F-127, 1 mM TCEP, and 2 mM EDTA in water. Protein and substrate were diluted in assay buffer.

Protein and substrate solutions were transferred to 1536-well plates (Black solid bottom 1536 microplate, HiBase; Cat. No. 789176-A Greiner Bio-One) by means of a GNF Systems WDII washer. Compounds were transferred using an Echo Liquid Handler (Echo 555, Labcyte). For plate measurements, an Envision reader (Product number 2104-0010, Perkin Elmer) was used. The instrument was equipped with filters and a dichoic mirror for measuring fluorescence polarization-type assays (Product numbers 2100-4070, 2100-5040, 2100-5140, and 2100-5150). The fluorescence of the Bodipy FL-based probe was excited at 480 nm and the emissions of the product were measured at 535 nm. The fluorescence in each well was excited by 30 flashes per measurement.

For the determination of AC₅₀ values, assays were performed at room temperature in 1536-well plates with a total assay volume of 6.03 μL per well. The test compounds were dissolved and diluted in 100% DMSO. For the assays, 30 nL of DMSO or compound solution were added per well, followed by the addition of 3 μL protein solution (80 nM protein in 1× assay buffer).

The final assay concentration of the protein was nominally 40 nM. After 45 minutes of pre-incubation at room temperature, the competition for binding was started by the addition of 3 μL probe solution (10 nM probe dissolved in assay buffer). The final concentration of the probe in the assays was 5 nM. After the addition of the probe solution, the final DMSO concentration in the assay was 0.5% (v/v). The effect of the probe on the pre-established compound-protein complex equilibrium was determined after 45 minutes (t=45 min). The AC₅₀ value was calculated from the plot of percentage of protein saturation versus the test compound concentration by a logistics fit according to: y=A²+(A¹−A²)/(1+(x/AC₅₀)^{p}), where y is the %-saturation value at the test compound concentration, x. A¹ is the lowest saturation value, i.e., 0%, and A² the maximum saturation value, i.e. 100%. The exponent, p, is the Hill coefficient.

### CRBN Assay Format 2b:

Assay conditions are similar to Format 2a with the exception of
a. The assay run in quadruplicate
b. PHERAstar reader (BMG Labtech, Offenburg, Germany) was used instead of an Envision reader
c. For the determination of AC₅₀ values, assays were performed at room temperature in 1536-well plates with a total assay volume of 8.03 μL per well. The test compounds were dissolved and diluted in 100% DMSO. For the assays, 30 nL of DMSO or compound solution were added per well, followed by the addition of 3 μL protein solution (80 nM protein in 1× assay buffer).

TABLE 4
CRBN Displacement Assay Results

| | CRBN AC₅₀ | CRBN AC₅₀ | CRBN Amax* | CRBN AC₅₀ |
|---|---|---|---|---|
| | [μM] | [μM] | (% at 50 μM) | [μM] |
| Compound | Format 1 | Format 2a | Format 2a | Format 2b |
| ILB-1 | 11.03 | 4.79 | | |
| ILB-3 | 14.10 | 4.17 | | |
| ILB-6 | 19.75 | 8.36 | | |
| ILB-7 | 61.24 | >50 | 40 | |
| ILB-8 | >100 | >50 | 21 | |
| ILB-9 | | >50 | 1 | |
| ILB-10 | | >50 | 0 | |
| ILB-13 | | 2.42 | | |
| ILB-14 | 13.02 | 5.79 | | |
| ILB-15 | 7.37 | 2.61 | | |
| ILB-16 | 11.71 | 7.34 | | |
| ILB-17 | | 3.41 | | |
| ILB-18 | 11.23 | 3.79 | | |
| ILB-19 | 2.51 | 0.80 | | |
| ILB-20 | 5.37 | 2.01 | | |
| ILB-22 | | 0.69 | | |
| ILB-24 | 2.85 | 0.68 | | |
| ILB-25 | 1.98 | 0.48 | | |
| ILB-26 | 2.92 | 1.26 | | |
| ILB-27 | 3.03 | 0.74 | | |
| ILB-28 | 3.41 | 1.30 | | |
| ILB-29 | 0.95 | | | |
| ILB-31 | 9.64 | 5.10 | | |
| ILB-33 | 6.52 | 2.68 | | |
| ILB-34 | 3.65 | 1.91 | | |
| ILB-38 | 8.44 | 6.02 | | |
| ILB-39 | | 3.15 | | |
| ILB-40 | 12.32 | 3.45 | | |
| ILB-43 | 2.99 | 2.93 | | |
| ILB-44 | | 5.43 | | |
| ILB-45 | 13.47 | | | |
| ILB-49 | | 3.36 | | |
| ILB-53 | 14.94 | 2.16 | | |
| ILB-55 | 14.41 | 4.04 | | |
| ILB-56 | 1.26 | 0.23 | | |
| ILB-59 | 1.36 | 0.34 | | |
| ILB-60 | 2.80 | | | |
| ILB-61 | | 3.44 | | |
| ILB-65 | | 26.15 | | |
| ILB-66 | | >50 | 46 | |
| ILB-68 | | 4.70 | | |
| ILB-70 | | >50 | 43 | |
| ILB-71 | | 6.01 | | 8.00 |
| ILB-72 | | 29.37 | | 18.02 |
| ILB-73 | | 15.05 | | 11.89 |
| ILB-74 | | 6.28 | | 4.61 |
| ILB-75 | | >50 | 40 | 28.69 |
| ILB-76 | | 8.09 | | 15.32 |
| ILB-77 | | 0.50 | | 0.30 |
| ILB-78 | | 5.72 | | 5.07 |
| ILB-79 | | 7.15 | | 6.78 |
| ILB-80 | | 9.41 | | 8.34 |
| ILB-81 | 12.33 | 2.38 | | |
| ILB-82 | | 29.77 | | |
| ILB-83 | | 45.61 | | |
| ILB-84 | | 33.33 | | |
| ILB-85 | | >50 | 47 | |
| ILB-86 | | 34.80 | | |
| ILB-87 | | 5.34 | | |
| ILB-88 | | >50 | 37 | |
| ILB-89 | | 16.71 | | |
| ILB-90 | | >50 | 40 | |
| ILB-91 | | 5.22 | | |
| ILB-92 | | 1.85 | | |
| ILB-93 | | 10.91 | | |
| ILB-94 | | 4.95 | | |

*For compounds tested in format 2a, with AC₅₀ values determined to be >50 μM, the values for the greatest effect (Amax) expressed as % displacement of the reference probe at the highest concentration tested (% at 50 μM) are provided in column 4.

### BTK-GFP, CSK, ABL2, EPHA4 and YES1 Protein Abundance Flow Cytometry Assay in HEK293A:

Degradation of BTK, CSK, ABL2, EPHA4 and YES1 was measured in HEK293A cells (Invitrogen R70507) expressing either BTK-GFP and RFP, CSK-GFP and mCherry, GFP-ABL2 and mCherry, EPHA4-GFP and mCherry, or YES1-GFP and mCherry from a stably integrated bicistronic BTK-GFP-iresRFP, CSK-GFP-CHYSEL-mCherry, GFP-ABL2-CHYSEL-mCherry, EPHA4-GFP-CHYSEL-mCherry, or YES1-GFP-CHYSEL-mCherry construct, respectively. Reduction of the GFP signal measured by flow cytometry served as readout for BTK, CSK, ABL2, EPHA4 and YES1 degradation after degrader treatment.

(A) Cloning of the pLenti6-BTK-GFP-Ires-RFP Sensor Vectors

The bicistronic BTK-GFP-iresRFP construct is based on a pLenti6-DEST vector backbone where GFP was introduced into the unique Xho I site downstream of the destination cassette (DEST) and RFP was cloned behind an internal ribosomal entry site (Ires).

In detail, the sensor construct was engineered by replacing NanoLuciferase (NLuc) by GFP and FireFly luciferase (FF) by RFP from pLenti6-DEST-NLuc-Ires-FF.

The pLenti6-DEST-NLuc-Ires-FF sensor construct was cloned by replacing eGFP from pLenti6-DEST-Ires-eGFP with a synthesized stuffer element (encoding Ires-FF with FF flanked by two Nhe I restriction sites) using blunt end cloning replacing Ires-eGFP between the two Pml I. To enable C-terminal tagging with NanoLuciferase (NLuc), NLuc was amplified from pNL1.1 (Promega #N1001) using linker primers with Xho1 sites for ligating into linearized pLenti6-DEST-Ires-FF using Xho1 digest resulting in the construct pLenti6-DEST-NLuc-Ires-FF.

The pLenti6-DEST-NLuc-Ires-FF served as base vector for cloning pLenti6-DEST-GFP-Ires-RFP using Gibson assembly to replace FF with RFP and NLuc with GFP. In a first round FF was replaced by RFP by amplifying RFP from a template using the following Gibson assembly linker primers to clone into pLenti6-DEST-NLuc-Ires-FF digested with Nhe1 and gel-purified to remove the FF fragment.

Gibson-Nhe1 RFPfw:
SEQ ID NO: 1
CGATGAATTCGCCACCgctagcATGGTGAGCAAGGGCGAGGAGC
Gibson-Nhe1 RFP-Stoprev:
SEQ ID NO: 2
CTCATTACTAACCGGctagcTTACTTGTACAGCTCGTCCATGC

The resulting pLenti6-DEST-NLuc-Ires-RFP vector served as the template to replace NLuc with GFP by amplifying GFP from a template using following Gibson assembly linker primers to clone into pLenti6-DEST-NLuc-Ires-RFP digested with Xho1 and gel-purified to remove the NLuc fragment.

Gibson-Xho1 GFPfw
SEQ ID NO: 3
CCAGCACAGTGGCGGCCGCTCGAGcATGGTGAGCAAGGGCGAGGAGCTGT
TCACC
Gibson-Xho1 GFP-Stoprev
SEQ ID NO: 4
CCGCGGGCCCTCTAGACTCGAGTTACTTGTACAGCTCGTCCATGCCGAGA
GT

All Gibson assembly reactions were performed with Gibson assembly Master Mix (New England Biolabs NEB E2611L) according to manufacturer's manual, resulting in the destination vector pLenti6-DEST-GFP-Ires-RFP to allow Gateway cloning.

To enable gateway cloning and C-terminal GFP tagging of BTK, the BTK open reading frame (ORF) was first shuttled from a pcDNA-DEST40-BTK vector (Invitrogen library ID INV_20090504v1) into pDONR221 (Invitrogen 12536-017) vector using a gateway BP reaction according to the manufacturer's manual (Invitrogen 11789-013) resulting in the novel construct pENTR221-BTK. For C-terminal tagging the STOP codon was mutated to a leucine performing a mutagenesis reaction with the following primers using the QuikChange Lightning mutagenesis kit (Agilent Technologies #210518) according to the manufacturer's manual, resulting in pENTR221-BTK (STOP-Leu).

pENTR221-BTK Quikchange STOP-Leu fw
SEQ ID NO: 5
gtcatggatgaagaatccTTGaacccagctttcttgtac
pENTR221-BTK Quikchange STOP-Leu REVC
SEQ ID NO: 6
gtacaagaaagctgggttCAAggattcttcatccatgac

To get the final pLenti6-BTK-GFP-Ires-RFP sensor construct, a Gateway LR reaction was performed between pLenti6-DEST-GFP-Ires-RFP and pENTR221-BTK (STOP-Leu) using the LR Clonase kit (Invitrogen 11791-019) according to the manufacturer's manual. All vectors described above have been sequenced for verification.

(B) Cloning of the pLenti6-CSK-GFP-CHYSEL-mCherry, mCherry-CHYSEL-GFP-ABL2, BTK(C481S)-GFP-CHYSEL-mCherry, -EPHA4-GFP-CHYSEL-mCherry and -YES1-GFP-CHYSEL-mCherry Sensor Vectors

The bicistronic GFP-CHYSEL-mCherry constructs are based on a pLenti6-DEST vector backbone where two cassettes were introduced: Xho1-EGFPCHYSEL-mCherry-Xho1 or Spe1-mCherryCHYSEL-EGFP-Spe1 to generate either pL6-CMV-DEST-GFP-CHYSEL-mCherry or pL6-CMV-mCherry-CHYSEL-GFP-DEST destination vectors for LR cloning, respectively. Both cassettes were synthesized by an external vendor (GeneART) and cloned into linearized pLenti6-DEST vector using Gibson assembly (GA) according to manufacturer's manual (New England Biolabs E5510).

| Xho1-EGFPCHY-mCherry | |
|---|---|
| (SEQ ID NO: 7) | |
| 1 | GATATCCAGC ACAGTGGCGG CCGCTCGAGC ATGGTGAGCA AGGGCGAGGA |
| | GCTGTTCACC |
| 61 | GGGGTGGTGC CCATCCTGGT CGAGCTGGAC GGCGACGTAA ACGGCCACAA |
| | GTTCAGCGTG |
| 121 | TCCGGCGAGG GCGAGGGCGA TGCCACCTAC GGCAAGCTGA CCCTGAAGTT |
| | CATCTGGACC |
| 181 | ACCGGCAAGC TGCCCGTGCC CTGGCCCACC CTCGTGACCA CCCTGACCTA |
| | CGGCGTGCAG |
| 241 | TGCTTCAGCC GCTACCCCGA CCACATGAAG GAGCACGAGT TCTTCAAGTC |
| | CGCCATGCCC |
| 301 | GAAGGCTACG TCCAGGAGGG CACCATCTTC TTCAAGGAGG ACGGCAACTA |
| | CAAGACCCGC |
| 361 | GCCGAGGTGA AGTTCGAGGG CGACACCCTG GTGAACCGCA TCGAGCTGAA |
| | GGGCATCGAC |
| 421 | TTCAAGGAGG ACGGCAACAT CCTGGGGCAC AAGCTGGAGT ACAACTACAA |
| | CAGCCACAAC |
| 481 | GTCTATATCA TGGCCGACAA GCAGAAGAAC GGCATCAAGG TGAACTTCAA |
| | GATCCGCCAC |
| 541 | AACATCGAGG ACGGCAGCGT GCAGCTCGCC GACCACTACC AGCAGAACAC |
| | CCCCATCGGC |
| 601 | GACGGCCCCG TGCTGCTGCC CGACAACCAC TACCTGAGCA CCCAGTCCGC |
| | CCTGAGCAAA |
| 661 | GACCCCAACG AGAAGCGCGA TCACATGGTC CTGCTGGAGT TCGTGACCGC |
| | CGCCGGGATC |
| 721 | ACTCTCGGCA TGGACGAGCT GTACAAGGGA AGCGGAGCGA CGAATTTTAG |
| | TCTACTGAAA |
| 781 | CAAGCGGGAG ACGTGGAGGA AAACCCTGGA CCTATGGTGA GCAAGGGCGA |
| | GGAGGATAAC |
| 841 | ATGGCCATCA TCAAGGAGTT CATGCGCTTC AAGGTGCACA TGGAGGGCTC |
| | CGTGAACGGC |
| 901 | CACGAGTTCG AGATCGAGGG CGAGGGCGAG GGCCGCCCCT ACGAGGGCAC |
| | CCAGACCGCC |
| 961 | AAGCTGAAGG TGACCAAGGG TGGCCCCCTG CCCTTCGCCT GGGACATCCT |
| | GTCCCCTCAG |
| 1021 | TTCATGTACG GCTCCAAGGC CTACGTGAAG CACCCCGCCG ACATCCCCGA |
| | CTACTTGAAG |
| 1081 | CTGTCCTTCC CCGAGGGCTT CAAGTGGGAG CGCGTGATGA ACTTCGAGGA |
| | CGGCGGCGTG |
| 1141 | GTGACCGTGA CCCAGGACTC CTCCCTGCAG GACGGCGAGT TCATCTACAA |
| | GGTGAAGCTG |
| 1201 | CGCGGCACCA ACTTCCCCTC CGACGGCCCC GTAATGCAGA AGAAGACCAT |
| | GGGCTGGGAG |
| 1261 | GCCTCCTCCG AGCGGATGTA CCCCGAGGAC GGCGCCCTGA AGGGCGAGAT |
| | CAAGGAGAGG |
| 1321 | CTGAAGCTGA AGGACGGCGG CCACTACGAC GCTGAGGTCA AGACCACCTA |
| | CAAGGCCAAG |
| 1381 | AAGCCCGTGC AGCTGCCCGG CGCCTACAAC GTCAACATCA AGTTGGACAT |
| | CACCTCCCAC |
| 1441 | AAGGAGGACT ACACCATCGT GGAACAGTAC GAACGCGCCG AGGGCCGCCA |
| | CTCCACCGGC |
| 1501 | GGCATGGACG AGCTGTACAA GTAGCTCGAG TCTAGAGGGC CCGCGGTTAA C |
| Spe1-mCherryCHY-EGFP ORIGIN | |
| (SEQ ID NO: 8) | |
| 1 | AGTACTCTAG AGGATCCACT AGTCGCCACC ATGGTGAGCA AGGGCGAGGA |
| | GGATAACATG |
| 61 | GCCATCATCA AGGAGTTCAT GCGCTTCAAG GTGCACATGG AGGGCTCCGT |
| | GAACGGCCAC |
| 121 | GAGTTCGAGA TCGAGGGCGA GGGCGAGGGC CGCCCCTACG AGGGCACCCA |
| | GACCGCCAAG |
| 181 | CTGAAGGTGA CCAAGGGTGG CCCCCTGCCC TTCGCCTGGG ACATCCTGTC |
| | CCCTCAGTTC |
| 241 | ATGTACGGCT CCAAGGCCTA CGTGAAGCAC CCCGCCGACA TCCCCGACTA |
| | CTTGAAGGTG |
| 301 | TCCTTCCCCG AGGGCTTCAA GTGGGAGCGC GTGATGAACT TCGAGGAGGG |
| | CGGCGTGGTG |
| 361 | ACCGTGACCC AGGACTCCTC CCTGCAGGAC GGCGAGTTCA TCTACAAGGT |
| | GAAGCTGCGC |
| 421 | GGCACCAACT TCCCCTCCGA CGGCCCCGTA ATGCAGAAGA AGACCATGGG |
| | CTGGGAGGCC |
| 481 | TCCTCCGAGC GGATGTACCC CGAGGACGGC GCCCTGAAGG GCGAGATCAA |
| | GCAGAGGCTG |
| 541 | AAGCTGAAGG ACGGCGGCCA CTACGACGCT GAGGTCAAGA CCACCTACAA |
| | GGCCAAGAAG |
| 601 | CCCGTGCAGC TGCCCGGCGC CTACAACGTC AACATCAAGT TGGACATCAC |
| | CTCCCACAAC |
| 661 | GAGGACTACA CCATCGTGGA ACAGTACGAA CGCGCCGAGG GCCGCCACTC |
| | CACCGGCGGC |
| 721 | ATGGACGAGC TGTACAAGGG AAGCGGAGCG ACGAATTTTA GTCTACTGAA |
| | ACAAGCGGGA |
| 781 | GACGTGGAGG AAAACCCTGG ACCTATGGTG AGCAAGGGCG AGGAGCTGTT |
| | CACCGGGGTG |
| 841 | GTGCCCATCC TGGTCGAGCT GGACGGCGAC GTAAACGGCC ACAAGTTCAG |
| | CGTGTCCGGC |
| 901 | GAGGGCGAGG GCGATGCCAC CTACGGCAAG CTGACCCTGA AGTTCATCTG |
| | CACCACCGGC |
| 961 | AAGCTGCCCG TGCCCTGGCC CACCCTCGTG ACCACCCTGA CCTACGGCGT |
| | GCAGTGCTTC |
| 1021 | AGCCGCTACC CCGACCACAT GAAGCAGCAC GACTTCTTCA AGTCCGCCAT |
| | GCCCGAAGGC |
| 1081 | TACGTCCAGG AGCGCACCAT CTTCTTCAAG GAGGACGGCA ACTACAAGAC |
| | CCGCGCCGAG |
| 1141 | GTGAAGTTCG AGGGCGACAC CCTGGTGAAC CGCATCGAGC TGAAGGGCAT |
| | CGACTTCAAG |
| 1201 | GAGGACGGCA ACATCCTGGG GCACAAGCTG GAGTACAACT ACAACAGCCA |
| | CAACGTCTAT |
| 1261 | ATCATGGCCG ACAAGCAGAA GAACGGCATC AAGGTGAACT TCAAGATCCG |
| | CCACAACATC |
| 1321 | GAGGACGGCA GCGTGCAGCT CGCCGACCAC TACCAGCAGA ACACCCCCAT |
| | CGGCGACGGC |
| 1381 | CCCGTGCTGC TGCCCGACAA CCACTACCTG AGCACCCAGT CCGCCCTGAG |
| | CAAAGACCCC |
| 1441 | AACGAGAAGC GCGATCACAT GGTCCTGCTG GAGTTCGTGA CCGCCGCCGG |
| | GATCACTCTC |
| 1501 | GGCATGGACG AGCTGTACAA GGCACTAGTC CAGTGTGGTG GAATTCTGCA |
| | GATATC |

Linearisation of pLenti6-DEST with Xho1 and GA with Xho1-EGFPCHYSEL-mCherry-Xho1 fragment resulted in gateway compatible pLenti6-DEST-EGFPCHYSEL-mCherry vector, linearisation of pLenti6-DEST with Spe1 and GA with Spe1-mCherryCHYSEL-EGFP-Spe1 fragment resulted in gateway compatible pLenti6-mCherryCHYSEL-EGFP-DEST vector.

pLenti6-mCherryCHYSEL-EGFP-ABL2 was generated by gateway LR cloning between pENTR221-ABL2 and pLenti6-mCherryCHYSEL-EGFP-DEST vectors. To clone the pLenti6-BTK(C481S)-GFP-CHYSEL-mCherry, pLenti6-CSK-GFP-CHYSEL-mCherry, -EPHA4-GFP-CHYSEL-mCherry and -YES1-GFP-CHYSEL-mCherry sensor vectors, the STOP codon had first to be mutated to a Leucine using a Quikchange reaction on existing pENTR221-CSK, -EPHA4 and -YES vectors using following primers resulting in novel vectors pENTR221-CSK(STOP-Leu), pENTR221-EPHA4(STOP-Leu) and pENTR221-YES(STOP-Leu).

pENTR221_CSK_Quikchange_STOP-LEU_fw
SEQ ID NO: 9
caagaaagctgggttcaacaggtgcagctcgtg
pENTR221_CSK_Quikchange_STOP-LEU_rev
SEQ ID NO: 10
cacgagctgcacctgttgaacccagctttcttg
pENTR221_EPHA4_QuikchangeSTOP-LEU_fw
SEQ ID NO: 11
gtacaagaaagctgggttcaagacgggaaccattctg
pENTR221_EPHA4_QuikchangeSTOP-LEU_rev
SEQ ID NO: 12
cagaatggttcccgtcttgaacccagctttcttgtac
pENTR221_YES_Quikchange_STOP-LEU_fw
SEQ ID NO: 13
gaaagctgggttcaataaattttctcctggctggtac
pENTR221_YES_Quikchange_STOP-LEU_rev
SEQ ID NO: 14
gtaccagccaggagaaaatttattgaacccagctttc

LR gateway cloning between pLenti6-DEST-GFP-CHYSEL-mCherry with pENTR221-CSK(STOP-Leu) or pENTR221-EPHA4(STOP-Leu), pENTR221-BTK(C481S)(STOP-Leu) or pENTR221-YES(STOP-Leu) vectors resulted in pLenti6-CSK-GFP-CHYSEL-mCherry, -EPHA4-GFP-CHYSEL-mCherry and -YES1-GFP-CHYSEL-mCherry sensor vectors, respectively. All vectors described were sequenced for verification.

### (C) Engineering of Stably Expressing 293A BTK-GFP-Ires-RFP, CSK-GFP-CHYSEL-mCherry, GFP-ABL2-CHYSEL-mCherry, EPHA4-GFP-CHYSEL-mCherry, or YES1-GFP-CHYSEL-mCherry Construct Sensor Cells

293A BTK-GFP-Ires-RFP, CSK-GFP-CHYSEL-mCherry, GFP-ABL2-CHYSEL-mCherry, EPHA4-GFP-CHYSEL-mCherry, or YES1-GFP-CHYSEL-mCherry sensor cells were generated by lentiviral vector transduction using the pLenti6-BTK-GFP-Ires-RFP, pLenti6-mCherryCHYSEL-EGFP-ABL2, pLenti6-CSK-GFP-CHYSEL-mCherry, pLenti6-EPHA4-GFP-CHYSEL-mCherry or pLenti6-YES1-GFP-CHYSEL-mCherry sensor construct described before. Lentiviral particles were produced in HEK293FT cells (Invitrogen R70007) by co-transfection of 500 ng pLenti6-BTK-GFP-Ires-RFP or pLenti6-IKZF3-GFP-Ires-RFP, 500 ng delta8.71 and 200 ng pVSVG diluted in 100 μL OptiMEM serum free medium (Invitrogen #11058-021) that was mixed after 5 min preincubation with 3 μL of Lipofectamine2000 (Invitrogen #11668-019) in 97 μL OptiMEM serum free medium. The mix was incubated for another 20 min at RT and then added on 1 mL of a freshly prepared suspension of HEK293FT cells in a well of a 6-well plate (concentration 1.2×10⁶ cells/mL). 1 day after transfection, the medium was replaced with 1.5 mL of complete growth medium (DMEM high Glucose+10% FCS+1% L-Glutamine+1% NEAA+1% NaPyr.). 48 h post transfection supernatant containing viral transducing particles was collected and frozen at −80° C.

2 days before transduction with viral particles 1×105 HEK293A cells (Invitrogen R70507) were seeded in 2 mL growth medium in a well of a 6-well plate. Infection was performed with 90 μL of collected supernatant containing viral transducing particles in 1 mL medium including 8 g/mL polybrene. 24 h post infection, stably transfected cells were selected with blasticidin at a concentration of 8 μg/mL referred to as stable HEK293A sensor cells.

### (D) Quantitative BTK-GFP, CSK-GFP, GFP-ABL2, EPHA4-GFP and YES1-GFP Abundance Measurements in Stable HEK293A Sensor Cells

Stable HEK293A sensor cells were maintained in complete growth medium (DMEM high Glucose+10% FCS+1% L-Glutamine+1% NEAA+1% NaPyr.) with passaging performed twice per week. On Day 0, stable HEK293A sensor cells were seeded at 10,000 cells/well in a 96-well microtiter plate in 260 μL complete medium. On Day 1, cells were treated in duplicate with 10-point 1:3 dilution series of compound using the HP D300 Digital Dispenser (Tecan). DMSO concentrations were normalized across the plate to 0.1%. On Day 2, after 24 h of incubation at 37° C., treatment media was discarded, cells rinsed with 100 μL/well PBS and then detached using 40 μL trypsin/well for 5 min. Trypsin was neutralized with 100 μL/well PBS+20% FCS). Flow cytometry was performed on the samples using the BD FACS CANTO II (Becton Dickinson). Cell identification was then performed using forward (FSC) vs. side scatter (SSC) plots. Single cell discrimination is performed using SSC-Width (SSC-W) vs. SSC-Height (SSC-H) plots. Median GFP values for 5,000 single cells are used to determine BTK levels. Median GFP values from HEK293 Å-iresRFP are used as a background signal and thus defining 0% BTK signal. Median GFP values from DMSO treated HEK293 Å-BTK-GFP-iresRFP are used to define 100% BTK signal for subsequent DC₅₀ curves (concentration at 50% BTK degradation). GFP and RFP are read in the channels called FITC and PE, respectively.

Concentration response curves plotting relative reduction of the GFP signal (measured by flow cytometry) versus 10 compound concentrations (starting concentration 10 μM, 3 fold dilution steps) of the compounds allowed generation of DC₅₀ values. Protein abundance measured for the second generation vectors having a chysel was done in close analogy to the above described method for measuring BTK abundance.

HEK293 cells Overexpressing hTNNI3K WT (Stable Clonal Line)

HEK293-hTNNI3K stable cells were seeded at 400,000 cells/well in 6-well plate and incubated at 37° C. and 5% CO₂ overnight in DMEM containing 10% FBS. The following day growth media was replaced with low-serum media containing DMEM with 0.5% FBS and incubated at 37° C. and 5% CO₂ overnight. Following overnight serum starvation, cells were treated with compounds at 20 μM starting dose with 1:5 serial dilution prepared in low-serum media. Media was removed and compounds were added to 6-well plate at 2 mL per well and cells were incubated at 37° C. and 5% CO₂ for 18 hours. Following compound treatment cells were washed with cold DPBS containing protease/phosphatase inhibitor cocktail (Thermo #1861284) and lysed in RIPA buffer on ice (Pierce #89900, also containing protease/phosphatase inhibitors). Following sonication and frequent vortexing at 4° C. for approximately 1 hour, samples were centrifuged at 14,000 rpm for 20 minutes. Supernatants were collected for protein samples. Protein concentration was determined using BioRad DC Protein Assay (FIGS. 3A and 3C).

Samples for western blotting were prepared in loading buffer (Thermo #NP0007) and 8 μg protein was loaded per lane in 4-12% Novex Bis-Tris gel and run at 60V for approximately 1 hour. Gels were transferred to PVDF membrane using semi-dry transfer device iBlot 2 from Thermo Scientific. Blots were blocked using Superblock T20 blocking solution for 2 hours at room temperature (Thermo #37536). Blots were then incubated with primary antibodies overnight with gentle shaking at 4° C. (TNNI3K #ab136954 and Beta-Actin CST #3700) at 1:1000 and 1:2000 dilution respectively. Following overnight primary antibody incubation, blots were washed 3 times on rocker for 5 minutes each with 1×TBS-T at room temperature. Blots were then incubated with secondary HRP-anti-rabbit at 1:2000 dilution (CST #7074) for 2 hours at room temperature. Blots were then washed with 1×TBS-T 3 times on rocker for 5 minutes each. Protein bands were detected using BioRad Chemi-Doc station and Supersignal West Dura substrate (Thermo #34075). Protein bands were quantified using ImageJ software. TNNI3K bands were normalized to beta-actin loading control. Data are graphed by fold change of cpd over DMSO control.

Table 5 columns are defined as follows: DC₅₀ refers to the concentration at which 50% maximal degradation was observed; deg Aₘₐₓ is the extent of degradation and the value refers to the % protein remaining at the concentration at which maximum degradation is seen. Compounds depicted in the table show BTK degradation with a broad range of degradation, ranking from partial degradation, e.g. >50% degradation (Aₘₐₓ<50) to more than 95% degradation (Aₘₐₓ<5). Compound 01 is a negative control in this table. Despite Compound 01 interacting with CRBN and BTK, no significant BTK degradation was observed, which is in line with the outcome of the in silico method described above, where ternary complex would not be enabled by this linker.

TABLE 5
BTK Degradation Assay Results

| | CRBN AC₅₀ | | BTK |
|---|---|---|---|
| | [μM] | BTK DC₅₀ | degradation |
| Compound | Format1 | [μM] | Aₘₐₓ |
| 01 | 1.593 | >3.3333 | 98 |
| 02 | 0.587 | 0.0021 | 4.5 |
| 03 | 0.257 | 0.0248 | 41.9 |
| 06 | 0.162 | 0.0047 | 2.1 |
| 07 | 0.496 | 0.0093 | 0.9 |
| 08 | 2.102 | >3.3333 | 55.1 |
| 09 | 1.403 | 0.0032 | 3.9 |
| 10 | 0.466 | 0.0033 | 3.1 |
| 11 | 0.599 | 0.0022 | 2.3 |
| 12 | 7.161 | 0.0107 | 2.3 |
| 13 | 0.575 | 0.0037 | 4.6 |
| 14 | 0.504 | 0.0259 | 25.9 |
| 15 | 4.107 | 0.0217 | 28.8 |
| 16 | 0.916 | 0.0226 | 15.9 |
| 17 | 3.552 | 0.1007 | 16.6 |
| 18 | 3.093 | 0.3073 | 7.6 |
| 19 | 0.104 | 0.2285 | 48.2 |
| 20 | >100 | 0.0277 | 37.1 |
| 23 | >50.5 | 0.0028 | 9.6 |
| 24 | 5.189 | 0.024 | 55.7 |
| 25 | 4.473 | >3.3333 | 51.1 |
| 26 | 3.92 | 0.0112 | 10.6 |
| 27 | 1.94 | 0.022 | 21.7 |
| 28 | nd | 0.0092 | 40 |
| 29 | 2.391 | 0.0402 | 40.5 |
| 30 | nd | 0.0098 | 12.2 |
| 31 | nd | 0.0557 | 32.8 |
| 32 | nd | 0.0014 | 16.7 |
| 33 | 0.915 | 0.0021 | 6.1 |
| 34 | 0.736 | 0.642 | 40.3 |
| 35 | 0.742 | 0.0021 | 6.6 |

### Discussion of Compounds 06 and 07 Degradation

Table 6 columns are defined as follows: DC₅₀ refers to the concentration at which 5000 maximal degradation was observed; deg Aₘₐₓ is the extent of degradation and the value refers to the % protein remaining at the concentration at which maximum degradation is seen. Compound 06 and Compound 07 show degradation of 4 target proteins CSK, ABL2, EPHA4 and YES 1 with a range of DC₅₀ values as depicted in the table.

TABLE 6

| | CSK | | ABL2 | | EPHA4 | | YES1 | |
|---|---|---|---|---|---|---|---|---|
| | DC₅₀ | | DC₅₀ | | DC₅₀ | | DC₅₀ | |
| Compound | [nM] | Aₘₐₓ | [nM] | Aₘₐₓ | [nM] | Aₘₐₓ | [nM] | Aₘₐₓ |
| 06 | 4 | 4 | 2 | 22 | 4 | 9 | 9 | 4 |
| 07 | 23 | 1 | 63 | 27 | 83 | 9 | 124 | 11 |

### Proteomics Experiment

FIG. 3E shows the volcano plots depicting the identification of degrader-dependent CRBN substrate candidates. HEK293 and TMD8 cells were treated for 6 hours with DMSO (3 replicates), 1 μM dasatinib (2 replicates), 1 μM compound 06 (3 replicates) and 1 μM compound 07 (3 replicates), respectively. TMT11plex-labeled peptides were generated with the PreOmics iST-NHS kit according to the manufacturer's protocol (PreOmics, Germany). The complexity of the samples was reduced by high pH fraction as described in Yang F et al. High-pH reversed-phase chromatography with fraction concatenation for 2D proteomic analysis. Expert Rev Proteomics. 2012, 9(2):129-134, and the resulting 72 fractions were pooled into 24 fractions. The 24 fractions were analyzed with a 25 cm×75 μm ID, 1.6 μm C18 Aurora Series emitter column (IonOpticks, Australia) on an EASY-nLC 1200 system coupled to an Orbitrap™ Fusion Lumos mass spectrometer (Thermo Fisher Scientific, USA). Data was acquired with a synchronous precursor selection method as described in McAlister G C et al. MultiNotch MS3 enables accurate, sensitive, and multiplexed detection of differential expression across cancer cell line proteomes. Anal Chem. 2014, 86(14):7150-7158. The Proteome Discoverer™ 2.1 software and the SEQUEST algorithm was used for protein identification and relative quantification. Log₂ fold changes of protein abundances and p-values were calculated in Python and R with the limma package (Ritchie M E et al. Limma powers differential expression analyses for RNA-sequencing and microarray studies. Nucleic Acids Res. 2015, 43(7):e47). The data show that bifunctional compounds of formula (I) can recruit and degrade multiple different targets. For instance, compounds 06 and 07 containing a promiscuous kinase binding motif, are able to degrade several of the targets to which they bind. By contrast, the dasatinib control molecule is an inhibitor that does not bear an E3-ligase binding motif and so does not appreciably degrade the targets to which it binds.

### Discussion of TNNI3K Degradation

FIGS. 4A-4B shows the amount of target protein (TNNI3K) that can be degraded by comparing initial levels of target protein TNNI3K before Compound 21 and Compound 22 treatment, respectively, in a concentration dependent matter. Both compounds showed full degradation. Starting at 6 nM and 32 nM no residual TNNI3K was detected. An alternative way to determine degradation is depicted in FIGS. 3A-3D.

Here the amount of total normalized TNNI3K expression levels were monitored upon compound treatment in a dose dependent manner. IC₅₀ refers to the concentration at which 50% reduction in protein expression was observed. A wide range of IC50s was observed. For example the experimentally observed IC50s ranked from 8.3 nM to 350 nM for Compound 22 and Compound 21, respectively.

Having thus described several aspects of several embodiments, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A bifunctional compound of Formula (I): 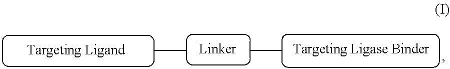
or a pharmaceutically acceptable salt, hydrate, solvate, prodrug, stereoisomer, or tautomer thereof, wherein:
the Targeting Ligand is a group that is capable of binding to a Target Protein;
the Linker is a group that covalently links the Targeting Ligand to the Targeting Ligase Binder; and
the Targeting Ligase Binder is a group that is capable of binding to a ligase (e.g., Cereblon E3 Ubiquitin ligase).

2. The bifunctional compound of claim 1, wherein the Targeting Ligase Binder has a Formula (TLB-I): 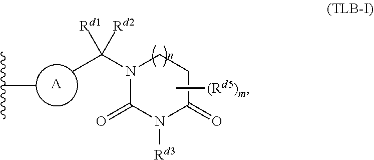
or a pharmaceutically acceptable salt, hydrate, solvate, prodrug, stereoisomer, or tautomer thereof, wherein: [symbol]
denotes the point of attachment to the Linker in Formula (I);
Ring A is a 6-membered aryl, or 5- or 6-membered heteroaryl, each of which is substituted with 0-4 occurrences of R^{d4};
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
or two R^{d5} together with the carbon atoms to which they are attached form a cycloalkyl;
or two R^{d5} attached to the same carbon atom form a C₃₋₄ spirocycloalkyl;
R^{p} is H or C₁₋₆ alkyl;
m is 1 or 2; and
n is 1 or 2.

3. The bifunctional compound of any one of the preceding claims, or a pharmaceutically acceptable salt, hydrate, solvate, prodrug, stereoisomer, or tautomer thereof, wherein n is 1.

4. The bifunctional compound of any one of the preceding claims, or a pharmaceutically acceptable salt, hydrate, solvate, prodrug, stereoisomer, or tautomer thereof, wherein R^{d3} is H.

5. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

6. The bifunctional compound of any one of the preceding claims, or a pharmaceutically acceptable salt, hydrate, solvate, prodrug, stereoisomer, or tautomer thereof, wherein ring A is selected from the group consisting of phenyl, pyridyl, pyridonyl, pyrazinyl, thiophenyl, pyrazolyl, imidazolyl, and pyrrolyl.

7. The bifunctional compound of any one of the preceding claims, or a pharmaceutically acceptable salt, hydrate, solvate, prodrug, stereoisomer, or tautomer thereof, wherein ring A is a 5-membered heteroaryl.

8. The bifunctional compound of any one of the preceding claims, or a pharmaceutically acceptable salt, hydrate, solvate, prodrug, stereoisomer, or tautomer thereof, wherein ring A is a 5-membered nitrogen-containing heteroaryl.

9. The bifunctional compound of any one of the preceding claims, or a pharmaceutically acceptable salt, hydrate, solvate, prodrug, stereoisomer, or tautomer thereof, wherein ring A is a 6-membered heteroaryl.

10. The bifunctional compound of any one of the preceding claims, or a pharmaceutically acceptable salt, hydrate, solvate, prodrug, stereoisomer, or tautomer thereof, wherein ring A is a 6-membered nitrogen-containing heteroaryl.

11. The bifunctional compound of any one of the preceding claims, or a pharmaceutically acceptable salt, hydrate, solvate, prodrug, stereoisomer, or tautomer thereof, wherein ring A is pyridyl or pyridonyl.

12. The bifunctional compound of any one of the preceding claims, or a pharmaceutically acceptable salt, hydrate, solvate, prodrug, stereoisomer, or tautomer thereof, wherein R^{d4} is hydroxyl or C₁₋₆ alkoxyl.

13. The bifunctional of any one of the preceding claims, wherein the Targeting Ligase Binder has a Formula (TLB-II): 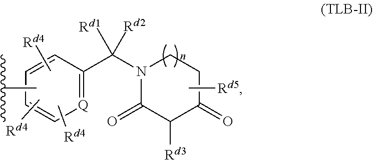
or a pharmaceutically acceptable salt, hydrate, solvate, prodrug, stereoisomer, or tautomer thereof, wherein: [symbol]
denotes the point of attachment to the Linker in Formula (I);
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

14. The bifunctional compound of any one of the preceding claims, or a pharmaceutically acceptable salt, hydrate, solvate, prodrug, stereoisomer, or tautomer thereof, wherein n is 1.

15. The bifunctional compound of any one of the preceding claims, or a pharmaceutically acceptable salt, hydrate, solvate, prodrug, stereoisomer, or tautomer thereof, wherein R^{d3} is H.

16. The bifunctional compound of any one of the preceding claims, or a pharmaceutically acceptable salt, hydrate, solvate, prodrug, stereoisomer, or tautomer thereof, wherein R^{d3} is
—CH₂OP(O)(OR^{p})₂.

17. The bifunctional compound of any one of the preceding claims, or a pharmaceutically acceptable salt, hydrate, solvate, prodrug, stereoisomer, or tautomer thereof, wherein R^{d4} is hydroxyl or C₁₋₆ alkoxyl.

18. The bifunctional compound of any one of the preceding claims, or a pharmaceutically acceptable salt, hydrate, solvate, prodrug, stereoisomer, or tautomer thereof, wherein the Targeting Ligase Binder has a Formula (TLB-III): 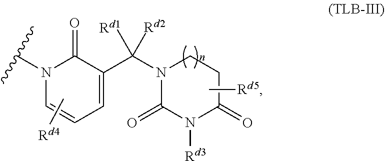
or a pharmaceutically acceptable salt, hydrate, solvate, prodrug, stereoisomer, or tautomer thereof, wherein: [symbol]
denotes the point of attachment to the Linker in Formula (I);
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

19. The bifunctional compound of any one of the preceding claims, or a pharmaceutically acceptable salt, hydrate, solvate, prodrug, stereoisomer, or tautomer thereof, wherein n is 1.

20. The bifunctional compound of any one of the preceding claims, or a pharmaceutically acceptable salt, hydrate, solvate, prodrug, stereoisomer, or tautomer thereof, wherein R^{d3} is H.

21. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

22. The bifunctional compound of any one of the preceding claims, or a pharmaceutically acceptable salt, hydrate, solvate, prodrug, stereoisomer, or tautomer thereof, wherein R^{d1} is H.

23. The bifunctional compound of any one of the preceding claims, or a pharmaceutically acceptable salt, hydrate, solvate, prodrug, stereoisomer, or tautomer thereof, wherein R^{d2} is H.

24. The bifunctional compound of any one of the preceding claims, or a pharmaceutically acceptable salt, hydrate, solvate, prodrug, stereoisomer, or tautomer thereof, wherein R^{d1} and R^{d2} are both H.

25. The bifunctional compound of any one of the preceding claims, wherein the Targeting Ligase Binder has a Formula (TLB-IV): 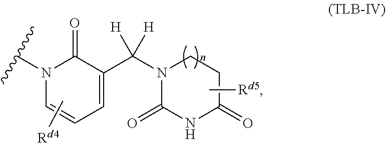
or a pharmaceutically acceptable salt, hydrate, solvate, prodrug, stereoisomer, or tautomer thereof, wherein: [symbol]
denotes the point of attachment to the Linker in Formula (I);
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl; and
n is 1 or 2.

26. The bifunctional compound of any one of the preceding claims, or a pharmaceutically acceptable salt, hydrate, solvate, prodrug, stereoisomer, or tautomer thereof, wherein n is 1.

27. The bifunctional compound of any one of the preceding claims, or a pharmaceutically acceptable salt, hydrate, solvate, prodrug, stereoisomer, or tautomer thereof, wherein R^{d3} is H.

28. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

29. The bifunctional compound of any one of the preceding claims, or a pharmaceutically acceptable salt, hydrate, solvate, prodrug, stereoisomer, or tautomer thereof, wherein R^{d4} is H or C₁₋₃ alkyl.

30. The bifunctional compound of any one of the preceding claims, or a pharmaceutically acceptable salt, hydrate, solvate, prodrug, stereoisomer, or tautomer thereof, wherein R^{d4} is H.

31. The bifunctional compound of any one of the preceding claims, or a pharmaceutically acceptable salt, hydrate, solvate, prodrug, stereoisomer, or tautomer thereof, wherein R^{d5} is H or C₁₋₃ alkyl.

32. The bifunctional compound of any one of the preceding claims, or a pharmaceutically acceptable salt, hydrate, solvate, prodrug, stereoisomer, or tautomer thereof, wherein R^{d5} is H.

33. The bifunctional compound of any one of the preceding claims, or a pharmaceutically acceptable salt, hydrate, solvate, prodrug, stereoisomer, or tautomer thereof, wherein the Targeting Ligase Binder has a Formula (TLB-V): 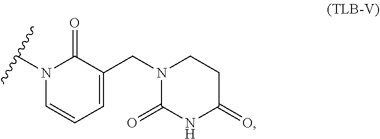 [SMILES: *n1cccc(CN2CCC(=O)NC2=O)c1=O]
or a pharmaceutically acceptable salt, hydrate, solvate, prodrug, stereoisomer, or tautomer thereof.

34. The bifunctional compound of any one of the preceding claims, or a pharmaceutically acceptable salt, hydrate, solvate, prodrug, stereoisomer, or tautomer thereof, wherein the Targeting Ligase Binder has a Formula (TLB-VI): 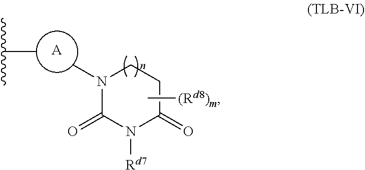
or a pharmaceutically acceptable salt, hydrate, solvate, prodrug, stereoisomer, or tautomer thereof, wherein: [symbol]
denotes the point of attachment to the Linker in Formula (I);
Ring A is a 6-membered aryl or 6-membered heteroaryl, each of which is independently substituted with 0-4 occurrences of R^{d6};
each R^{d6} is independently selected from the group consisting of H, hydroxyl, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{p} is H or C₁₋₆ alkyl;
each R^{d8} is independently selected from the group consisting of H, C₁₋₆ alkyl, halogen, C₁₋₆ haloalkyl, and C₁₋₆ heteroalkyl;
or two R^{d8} together with the carbon atoms to which they are attached form a cycloalkyl;
or two R^{d8} attached to the same carbon atom form a C₃₋₄ spirocycloalkyl;
m is 1 or 2; and
n is 1 or 2.

35. The bifunctional compound of any one of the preceding claims, or a pharmaceutically acceptable salt, hydrate, solvate, prodrug, stereoisomer, or tautomer thereof, wherein ring A is selected from the group consisting of phenyl, pyridyl, pyridonyl, pyrazinyl, thiophenyl, pyrazolyl, imidazolyl, and pyrrolyl.

36. The bifunctional compound of any one of the preceding claims, or a pharmaceutically acceptable salt, hydrate, solvate, prodrug, stereoisomer, or tautomer thereof, wherein ring A is a nitrogen-containing 6-membered heteroaryl.

37. The bifunctional compound of any one of the preceding claims, or a pharmaceutically acceptable salt, hydrate, solvate, prodrug, stereoisomer, or tautomer thereof, wherein ring A is pyridyl.

38. The bifunctional compound of any one of the preceding claims, or a pharmaceutically acceptable salt, hydrate, solvate, prodrug, stereoisomer, or tautomer thereof, wherein n is 1.

39. The bifunctional compound of any one of the preceding claims, or a pharmaceutically acceptable salt, hydrate, solvate, prodrug, stereoisomer, or tautomer thereof, wherein n is 2.

40. The bifunctional compound of any one of the preceding claims, or a pharmaceutically acceptable salt, hydrate, solvate, prodrug, stereoisomer, or tautomer thereof, wherein R^{d7} is —CH₂OP(O)(OR^{P})₂.

41. The bifunctional compound of any one of the preceding claims, or a pharmaceutically acceptable salt, hydrate, solvate, prodrug, stereoisomer, or tautomer thereof, wherein R^{d7} is H.

42. The bifunctional compound of any one of the preceding claims, or a pharmaceutically acceptable salt, hydrate, solvate, prodrug, stereoisomer, or tautomer thereof, wherein R^{d8} is H.

43. The bifunctional compound of any one of the preceding claims, or a pharmaceutically acceptable salt, hydrate, solvate, prodrug, stereoisomer, or tautomer thereof, wherein R^{d7} and R^{d8} are both H.

44. The bifunctional compound of any one of the preceding claims, or a pharmaceutically acceptable salt, hydrate, solvate, prodrug, stereoisomer, or tautomer thereof, wherein R^{d6} is H.

45. The bifunctional compound of any one of the preceding claims, or a pharmaceutically acceptable salt, hydrate, solvate, prodrug, stereoisomer, or tautomer thereof, wherein R^{d6} is selected from the group consisting of H, halogen, C₁₋₆ alkyl, and C₁₋₆ alkoxyl.

46. The bifunctional compound of any one of the preceding claims, or a pharmaceutically acceptable salt, hydrate, solvate, prodrug, stereoisomer, or tautomer thereof, wherein R^{d6} is selected from the group consisting of H, halogen, C₁₋₆ alkyl, and C₁₋₆ alkoxyl; and R^{d7}, and R^{d8} are each H.

47. The bifunctional compound of any one of the preceding claims, wherein the Targeting Ligase Binder has a Formula (TLB-VII): 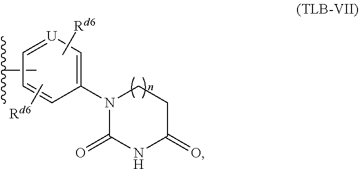
or a pharmaceutically acceptable salt, hydrate, solvate, prodrug, stereoisomer, or tautomer thereof, wherein: [symbol]
denotes the point of attachment to the Linker in Formula (I);
U is —CR^{d6} or N;
each R^{d6} is independently selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

48. The bifunctional compound of any one of the preceding claims, or a pharmaceutically acceptable salt, hydrate, solvate, prodrug, stereoisomer, or tautomer thereof, wherein n is 1.

49. The bifunctional compound of any one of the preceding claims, or a pharmaceutically acceptable salt, hydrate, solvate, prodrug, stereoisomer, or tautomer thereof, wherein n is 2.

50. The bifunctional compound of any one of the preceding claims, or a pharmaceutically acceptable salt, hydrate, solvate, prodrug, stereoisomer, or tautomer thereof, wherein each R^{d6} is independently selected from the group consisting of H, halogen, C₁₋₃ alkyl, and C₁₋₃ alkoxy.

51. The bifunctional compound of any one of the preceding claims, or a pharmaceutically acceptable salt, hydrate, solvate, prodrug, stereoisomer, or tautomer thereof, wherein each R^{d6} is H.

52. The bifunctional compound of any one of the preceding claims, or a pharmaceutically acceptable salt, hydrate, solvate, prodrug, stereoisomer, or tautomer thereof, wherein one of R^{d6} is H.

53. The bifunctional compound of any one of the preceding claims, or a pharmaceutically acceptable salt, hydrate, solvate, prodrug, stereoisomer, or tautomer thereof, wherein one of R^{d6} is not H.

54. The bifunctional compound of any one of the preceding claims, wherein the Targeting Ligase Binder has a Formula (TLB-VIII): 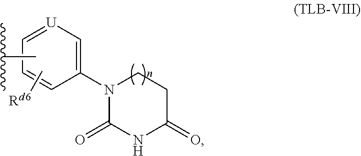
or a pharmaceutically acceptable salt, hydrate, solvate, prodrug, stereoisomer, or tautomer thereof, wherein: [symbol]
denotes the point of attachment to the Linker in Formula (I);
U is —CR^{d6} or N;
R^{d6} is selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

55. The bifunctional compound of any one of the preceding claims, or a pharmaceutically acceptable salt, hydrate, solvate, prodrug, stereoisomer, or tautomer thereof, wherein the Targeting Ligase Binder has a Formula (TLB-IX): 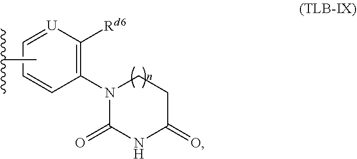
or a pharmaceutically acceptable salt, hydrate, solvate, prodrug, stereoisomer, or tautomer thereof, wherein: [symbol]
denotes the point of attachment to the Linker in Formula (I);
U is independently —CR^{d6} or N;
R^{d6} is selected from the group consisting of H, hydroxyl, C₁₋₆ alkyl, halogen, C₁₋₆ alkoxyl, C₁₋₆ haloalkyl, and C₁₋₆ heteroalkyl; and
n is 1 or 2.

56. The bifunctional compound of any one of the preceding claims, or a pharmaceutically acceptable salt, hydrate, solvate, prodrug, stereoisomer, or tautomer thereof, wherein n is 1.

57. The bifunctional compound of any one of the preceding claims, or a pharmaceutically acceptable salt, hydrate, solvate, prodrug, stereoisomer, or tautomer thereof, wherein n is 2.

58. The bifunctional compound of any one of the preceding claims, or a pharmaceutically acceptable salt, hydrate, solvate, prodrug, stereoisomer, or tautomer thereof, wherein U is N.

59. The bifunctional compound of any one of the preceding claims, or a pharmaceutically acceptable salt, hydrate, solvate, prodrug, stereoisomer, or tautomer thereof, wherein U is —CR^{d6}.

60. The bifunctional compound of any one of the preceding claims, or a pharmaceutically acceptable salt, hydrate, solvate, prodrug, stereoisomer, or tautomer thereof, wherein each R^{d6} is independently selected from the group consisting of H, methyl, halogen, methoxy, and methoxymethyl.

61. The bifunctional compound of any one of the preceding claims, or a pharmaceutically acceptable salt, hydrate, solvate, prodrug, stereoisomer, or tautomer thereof, wherein R^{d6} is H.

62. The bifunctional compound of any one of the preceding claims, or a pharmaceutically acceptable salt, hydrate, solvate, prodrug, stereoisomer, or tautomer thereof, wherein R^{d6} is methyl.

63. The bifunctional compound of any one of the preceding claims, or a pharmaceutically acceptable salt, hydrate, solvate, prodrug, stereoisomer, or tautomer thereof, wherein R^{d6} is halogen.

64. The bifunctional compound of any one of the preceding claims, or a pharmaceutically acceptable salt, hydrate, solvate, prodrug, stereoisomer, or tautomer thereof, wherein R^{d6} is methoxy.

65. The bifunctional compound of any one of the preceding claims, wherein the Linker has Formula (L-I): 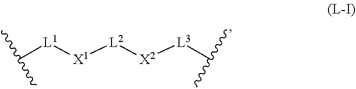
or a pharmaceutically acceptable salt, hydrate, solvate, prodrug, stereoisomer, or tautomer thereof, wherein:
L¹ is selected from the group consisting of a bond, O, NR′, C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand in Formula (I);
X¹ and X² are each independently selected from the group consisting of a bond, carbocyclyl, heterocyclyl, and heteroaryl;
L² is selected from the group consisting of a bond, O, NR′, C(O), C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from a bond, C₁₋₆ alkylene, C₂₋₆ alkenylene, C₂₋₆ alkynylene, C₁₋₆ heteroalkylene, C(O), S(O)₂, O, NR′, *C(O)—C₁₋₉ alkylene, and *C(O)—C₁₋₉ heteroalkylene, wherein * denotes the point of attachment of L³ to X² in (L-I),
wherein no more than 2 of L¹, X¹, X², L², and L³ can simultaneously be a bond; and
R′ is hydrogen or C₁₋₆ alkyl.

66. The bifunctional compound of any one of the preceding claims, or a pharmaceutically acceptable salt, hydrate, solvate, prodrug, stereoisomer, or tautomer thereof, wherein L³ is selected from the group consisting of a bond, —O—, —C(O)—, —S(O)₂—, C₁₋₆ alkylene, C₂₋₆ alkynylene, and C₁₋₆ heteroalkylene.

67. The bifunctional compound of any one of the preceding claims, or a pharmaceutically acceptable salt, hydrate, solvate, prodrug, stereoisomer, or tautomer thereof, wherein one of X¹ and X² is not a bond.

68. The bifunctional compound of any one of the preceding claims, or a pharmaceutically acceptable salt, hydrate, solvate, prodrug, stereoisomer, or tautomer thereof, wherein one of X¹ and X² is a bond, and the other is a carbocyclyl or heterocyclyl.

69. The bifunctional compound of any one of the preceding claims, or a pharmaceutically acceptable salt, hydrate, solvate, prodrug, stereoisomer, or tautomer thereof, wherein one of X¹ and X² is a bond, and the other is a heterocyclyl.

70. The bifunctional compound of any one of the preceding claims, or a pharmaceutically acceptable salt, hydrate, solvate, prodrug, stereoisomer, or tautomer thereof, wherein X¹ and X² are each independently selected from piperidinyl and piperazinyl.

71. The bifunctional compound of any one of the preceding claims, or a pharmaceutically acceptable salt, hydrate, solvate, prodrug, stereoisomer, or tautomer thereof, wherein X¹ and X² are both piperidinyl.

72. The bifunctional compound of any one of the preceding claims, or a pharmaceutically acceptable salt, hydrate, solvate, prodrug, stereoisomer, or tautomer thereof, wherein —X¹-L²-X²— is:

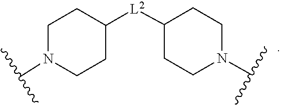

73. The bifunctional compound of any one of the preceding claims, wherein the Linker is a compound having the following formula: 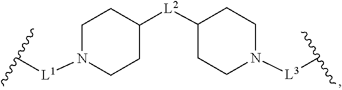
or a pharmaceutically acceptable salt, hydrate, solvate, prodrug, stereoisomer, or tautomer thereof.

74. The bifunctional compound of any one of the preceding claims, or a pharmaceutically acceptable salt, hydrate, solvate, prodrug, stereoisomer, or tautomer thereof, wherein —X¹-L²-X²— forms a spiroheterocyclyl having the structure, 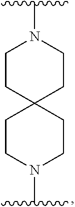 [SMILES: *N1CCC2(CC1)CCN(*)CC2]
substituted with 0-4 occurrences of R^{a}, wherein each R^{a} is independently selected from C₁₋₆ alkyl, C₁₋₆ alkoxyl, and C₁₋₆ hydroxyalkyl.

75. The bifunctional compound of any one of the preceding claims, or a pharmaceutically acceptable salt, hydrate, solvate, prodrug, stereoisomer, or tautomer thereof, wherein —X¹-L²-X²— forms a spiroheterocyclyl having the structure, 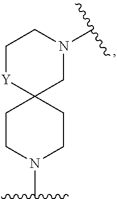
substituted with 0-4 occurrences of R^{b}, wherein Y is selected from CH₂, oxygen, and nitrogen; and each R^{b} is independently selected from C₁₋₆ alkyl, C₁₋₆ alkoxyl, and C₁₋₆ hydroxyalkyl.

76. The bifunctional compound of any one of the preceding claims, or a pharmaceutically acceptable salt, hydrate, solvate, prodrug, stereoisomer, or tautomer thereof, wherein X¹ and X² are each a bond.

77. The bifunctional compound of any one of the preceding claims, or a pharmaceutically acceptable salt, hydrate, solvate, prodrug, stereoisomer, or tautomer thereof, wherein L³ is independently selected from the group consisting of —C(O)—, C₂₋₆ alkynylene, or C₁₋₆ heteroalkylene; and L¹ is —C(O)—, C₁₋₈ alkylene, C₁₋₈ heteroalkylene, and *C₁₋₆ alkylene-C(O).

78. The bifunctional compound of any one of the preceding claims, or a pharmaceutically acceptable salt, hydrate, solvate, prodrug, stereoisomer, or tautomer thereof, wherein L³ is selected from the group consisting of —C(O)—, —O—C₁₋₆ alkylene, C₂₋₆ alkynylene, and C₁₋₆ heteroalkylene; and L¹ is C₁₋₈ alkylene or C₁₋₈ heteroalkylene.

79. The bifunctional compound of any one of the preceding claims, or a pharmaceutically acceptable salt, hydrate, solvate, prodrug, stereoisomer, or tautomer thereof, wherein L³ is —C(O)— or C₁₋₆ heteroalkylene; and L¹ is C₁₋₈ alkylene or C₁₋₈ heteroalkylene.

80. The bifunctional compound of any one of the preceding claims, or a pharmaceutically acceptable salt, hydrate, solvate, prodrug, stereoisomer, or tautomer thereof, wherein L³ is a bond or —O—; and L¹ is —C(O)— or C₁₋₈ heteroalkylene.

81. The bifunctional compound of any one of the preceding claims, or a pharmaceutically acceptable salt, hydrate, solvate, prodrug, stereoisomer, or tautomer thereof, wherein L³ is selected from the group consisting of —O—, —C(O)—, —S(O)₂—, and C₁₋₆ heteroalkylene; and L¹ is C₁₋₈ alkylene or C₁₋₈ heteroalkylene.

82. The bifunctional compound of any one of the preceding claims, or a pharmaceutically acceptable salt, hydrate, solvate, prodrug, stereoisomer, or tautomer thereof, wherein L² is —C(O)—, —NR′—, or C₁₋₆ alkylene.

83. The bifunctional compound of any one of the preceding claims, or a pharmaceutically acceptable salt, hydrate, solvate, prodrug, stereoisomer, or tautomer thereof, wherein L² is —C(O)—, —O—, or C₁₋₆ alkylene.

84. The bifunctional compound of any one of the preceding claims, or a pharmaceutically acceptable salt, hydrate, solvate, prodrug, stereoisomer, or tautomer thereof, wherein L² is C₁₋₆ alkylene.

85. The bifunctional compound of any one of the preceding claims, or a pharmaceutically acceptable salt, hydrate, solvate, prodrug, stereoisomer, or tautomer thereof, wherein L² is selected from the group consisting of —C(O)—, C₁₋₆ alkylene, C₁₋₆ heteroalkylene, and *C(O)NR′—C₁₋₆ alkylene.

86. The bifunctional compound of any one of the preceding claims, wherein the Targeting Ligase Binder-Linker has Formula (TLB-L-I): 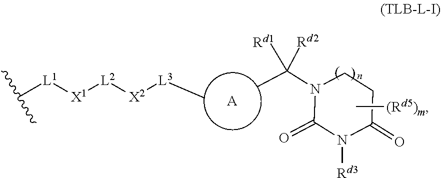
or a pharmaceutically acceptable salt, hydrate, solvate, prodrug, stereoisomer, or tautomer thereof, wherein: [symbol]
denotes the point of attachment to the Targeting Ligand in Formula (I);
L¹ is selected from the group consisting of a bond, —O—, —NR′—, —C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O), —S(O)₂—, —O—, *C(O)—C₁₋₉ alkylene, and *C(O)—C₁₋₉ heteroalkylene, wherein * denotes the point of attachment of L³ to X² in Formula (TLB-L-I);
wherein no more than 2 of L¹, X¹, X², L², and L³ can simultaneously be a bond;
Ring A is a 6-membered aryl, or 5- or 6-membered heteroaryl, each of which is substituted with 0-4 occurrences of R^{d4};
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
or two R^{d5} together with the carbon atoms to which they are attached form a cycloalkyl;
or two R^{d5} attached to the same carbon atom form a C₃₋₄ spirocycloalkyl;
R^{p} is H or C₁₋₆ alkyl;
m is 1 or 2; and
n is 1 or 2.

87. The bifunctional compound of any one of the preceding claims, or a pharmaceutically acceptable salt, hydrate, solvate, prodrug, stereoisomer, or tautomer thereof, wherein ring A is selected from the group consisting of phenyl, pyridyl, pyridonyl, pyrazinyl, thiophenyl, pyrazolyl, imidazolyl, and pyrrolyl.

88. The bifunctional compound of any one of the preceding claims, or a pharmaceutically acceptable salt, hydrate, solvate, prodrug, stereoisomer, or tautomer thereof, wherein ring A is a 5-membered heteroaryl.

89. The bifunctional compound of any one of the preceding claims, or a pharmaceutically acceptable salt, hydrate, solvate, prodrug, stereoisomer, or tautomer thereof, wherein ring A is a 5-membered nitrogen-containing heteroaryl.

90. The bifunctional compound of any one of the preceding claims, or a pharmaceutically acceptable salt, hydrate, solvate, prodrug, stereoisomer, or tautomer thereof, wherein ring A is a 6-membered heteroaryl.

91. The bifunctional compound of any one of the preceding claims, or a pharmaceutically acceptable salt, hydrate, solvate, prodrug, stereoisomer, or tautomer thereof, wherein ring A is a 6-membered nitrogen-containing heteroaryl.

92. The bifunctional compound of any one of the preceding claims, or a pharmaceutically acceptable salt, hydrate, solvate, prodrug, stereoisomer, or tautomer thereof, wherein ring A is pyridyl.

93. The bifunctional compound of any one of the preceding claims, or a pharmaceutically acceptable salt, hydrate, solvate, prodrug, stereoisomer, or tautomer thereof, wherein n is 1.

94. The bifunctional compound of any one of the preceding claims, or a pharmaceutically acceptable salt, hydrate, solvate, prodrug, stereoisomer, or tautomer thereof, wherein R^{d3} is H.

95. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

96. The bifunctional compound of any one of the preceding claims, wherein the Targeting Ligase Binder-Linker has Formula (TLB-L-II): 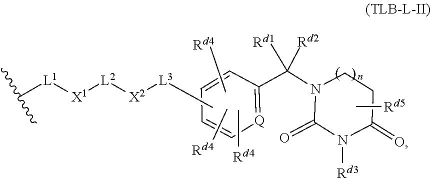
or a pharmaceutically acceptable salt, hydrate, solvate, prodrug, stereoisomer, or tautomer thereof, wherein: [symbol]
denotes the point of attachment to the Targeting Ligand in Formula (I);
L¹ is selected from the group consisting of a bond, —O—, —NR′—, —C(O), C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O), —S(O)₂—, —O—, *C(O)—C₁₋₉ alkylene, and *C(O)—C₁₋₉ heteroalkylene, wherein * denotes the point of attachment of L³ to X² in Formula (TLB-L-II);
wherein no more than 2 of L¹, X¹, X², L², and L³ can simultaneously be a bond;
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

97. The bifunctional compound of any one of the preceding claims, or a pharmaceutically acceptable salt, hydrate, solvate, prodrug, stereoisomer, or tautomer thereof, wherein n is 1.

98. The bifunctional compound of any one of the preceding claims, or a pharmaceutically acceptable salt, hydrate, solvate, prodrug, stereoisomer, or tautomer thereof, wherein R^{d3} is H.

99. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

100. The bifunctional compound of any one of the preceding claims, wherein the Targeting Ligase Binder-Linker has Formula (TLB-L-III): 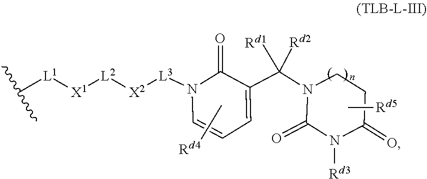
or a pharmaceutically acceptable salt, hydrate, solvate, prodrug, stereoisomer, or tautomer thereof, wherein: [symbol]
denotes the point of attachment to the Targeting Ligand in Formula (I);
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (TLB-L-III);
wherein no more than 2 of L¹, X¹, X², L², and L³ can simultaneously be a bond;
R′ is hydrogen or C₁₋₆ alkyl;
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

101. The bifunctional compound of any one of the preceding claims, or a pharmaceutically acceptable salt, hydrate, solvate, prodrug, stereoisomer, or tautomer thereof, wherein n is 1.

102. The bifunctional compound of any one of the preceding claims, or a pharmaceutically acceptable salt, hydrate, solvate, prodrug, stereoisomer, or tautomer thereof, wherein R^{d3} is H.

103. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

104. The bifunctional compound of any one of the preceding claims, wherein the Targeting Ligase Binder-Linker has Formula (TLB-L-IV): 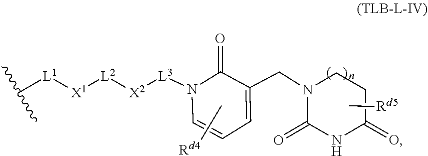
or a pharmaceutically acceptable salt, hydrate, solvate, prodrug, stereoisomer, or tautomer thereof, wherein: [symbol]
denotes the point of attachment to the Targeting Ligand in Formula (I);
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (TLB-L-IV);
wherein no more than 2 of L¹, X¹, X², L², and L³ can simultaneously be a bond;
R′ is hydrogen or C₁₋₆ alkyl;
R^{d4} is selected from the group consisting of H, C₁₋₆ alkyl, halogen, C₁₋₆ haloalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
n is 1 or 2.

105. The bifunctional compound of any one of the preceding claims, or a pharmaceutically acceptable salt, hydrate, solvate, prodrug, stereoisomer, or tautomer thereof, wherein n is 1.

106. The bifunctional compound of any one of the preceding claims, or a pharmaceutically acceptable salt, hydrate, solvate, prodrug, stereoisomer, or tautomer thereof, wherein n is 2.

107. The bifunctional compound of any one of the preceding claims, wherein the Targeting Ligase Binder-Linker has Formula (TLB-L-V): 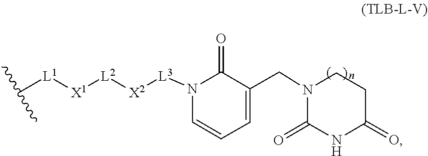
or a pharmaceutically acceptable salt, hydrate, solvate, prodrug, stereoisomer, or tautomer thereof, wherein: [symbol]
denotes the point of attachment to the Targeting Ligand in Formula (I);
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (TLB-L-V);
wherein no more than 2 of L¹, X¹, X², L², and L³ can simultaneously be a bond;
R′ is hydrogen or C₁₋₆ alkyl; and
n is 1 or 2.

108. The bifunctional compound of any one of the preceding claims, or a pharmaceutically acceptable salt, hydrate, solvate, prodrug, stereoisomer, or tautomer thereof, wherein n is 1.

109. The bifunctional compound of any one of the preceding claims, or a pharmaceutically acceptable salt, hydrate, solvate, prodrug, stereoisomer, or tautomer thereof, wherein n is 2.

110. The bifunctional compound of any one of the preceding claims, or a pharmaceutically acceptable salt, hydrate, solvate, prodrug, stereoisomer, or tautomer thereof, wherein L³ is selected from the group consisting of —O—, —C(O)—, —S(O)₂—, C₁₋₆ alkylene, C₂₋₆ alkynylene, and C₁₋₆ heteroalkylene.

111. The bifunctional compound of any one of the preceding claims, or a pharmaceutically acceptable salt, hydrate, solvate, prodrug, stereoisomer, or tautomer thereof, wherein one of X¹ and X² is not a bond.

112. The bifunctional compound of any one of the preceding claims, or a pharmaceutically acceptable salt, hydrate, solvate, prodrug, stereoisomer, or tautomer thereof, wherein one of X¹ and X² is a bond, and the other is a carbocyclyl or heterocyclyl.

113. The bifunctional compound of any one of the preceding claims, or a pharmaceutically acceptable salt, hydrate, solvate, prodrug, stereoisomer, or tautomer thereof, wherein one of X¹ and X² is a bond, and the other is a heterocyclyl.

114. The bifunctional compound of any one of the preceding claims, wherein the Targeting Ligase Binder-Linker, or a pharmaceutically acceptable salt, hydrate, solvate, prodrug, stereoisomer, or tautomer thereof, has a Formula selected from:

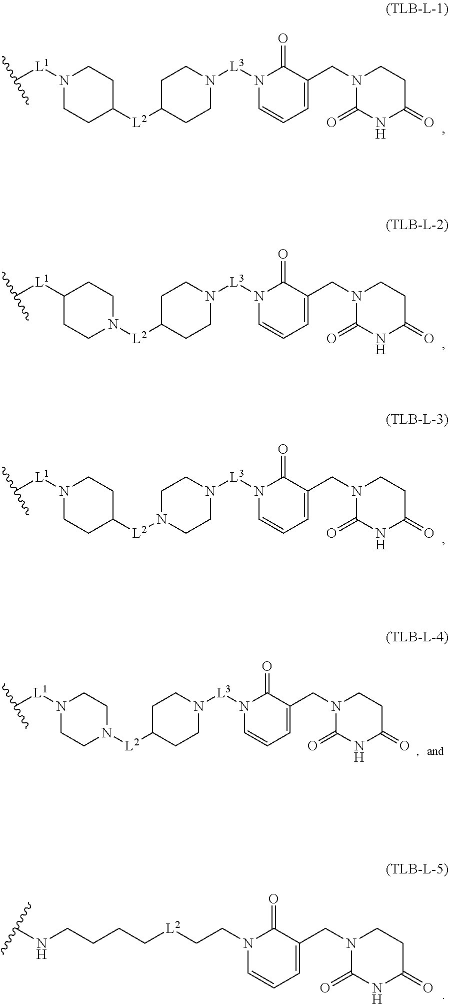

115. The bifunctional compound of any one of the preceding claims, wherein the compound has the Formula (BF-I): 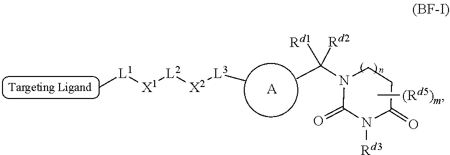
or a pharmaceutically acceptable salt, hydrate, solvate, prodrug, stereoisomer, or tautomer thereof, wherein:
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene;
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (BF-I);
wherein no more than 2 of L¹, X¹, X², L², and L³ can simultaneously be a bond;
Ring A is a 6-membered aryl, or 5- or 6-membered heteroaryl, each of which is substituted with 0-4 occurrences of R^{d4};
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, hydroxyl, C₁₋₆ alkyl, halogen, C₁₋₆ haloalkyl, C₁₋₆ alkoxyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
or two R^{d5} together with the carbon atoms to which they are attached form a cycloalkyl;
or two R^{d5} attached to the same carbon atom form a C₃₋₄ spirocycloalkyl;
R^{p} is H or C₁₋₆ alkyl;
m is 1 or 2; and
n is 1 or 2, wherein the Targeting Ligand is a group capable of binding to a Target Protein.

116. The bifunctional compound of any one of the preceding claims, or a pharmaceutically acceptable salt, hydrate, solvate, prodrug, stereoisomer, or tautomer thereof, wherein ring A is selected from the group consisting of phenyl, pyridyl, pyridonyl, pyrazinyl, thiophenyl, pyrazolyl, imidazolyl, and pyrrolyl.

117. The bifunctional compound of any one of the preceding claims, or a pharmaceutically acceptable salt, hydrate, solvate, prodrug, stereoisomer, or tautomer thereof, wherein ring A is a 5-membered heteroaryl.

118. The bifunctional compound of any one of the preceding claims, or a pharmaceutically acceptable salt, hydrate, solvate, prodrug, stereoisomer, or tautomer thereof, wherein ring A is a 5-membered nitrogen-containing heteroaryl.

119. The bifunctional compound of any one of the preceding claims, or a pharmaceutically acceptable salt, hydrate, solvate, prodrug, stereoisomer, or tautomer thereof, wherein ring A is a 6-membered heteroaryl.

120. The bifunctional compound of any one of the preceding claims, or a pharmaceutically acceptable salt, hydrate, solvate, prodrug, stereoisomer, or tautomer thereof, wherein ring A is a 6-membered nitrogen-containing heteroaryl.

121. The bifunctional compound of any one of the preceding claims, or a pharmaceutically acceptable salt, hydrate, solvate, prodrug, stereoisomer, or tautomer thereof, wherein ring A is pyridyl.

122. The bifunctional compound of any one of the preceding claims, or a pharmaceutically acceptable salt, hydrate, solvate, prodrug, stereoisomer, or tautomer thereof, wherein n is 1.

123. The bifunctional compound of any one of the preceding claims, or a pharmaceutically acceptable salt, hydrate, solvate, prodrug, stereoisomer, or tautomer thereof, wherein n is 2.

124. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

125. The bifunctional compound of any one of the preceding claims, or a pharmaceutically acceptable salt, hydrate, solvate, prodrug, stereoisomer, or tautomer thereof, wherein R^{d3} is H.

126. The bifunctional compound of any one of the preceding claims, wherein the compound has the Formula (BF-II): 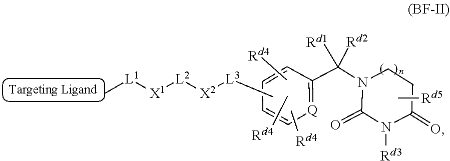
or a pharmaceutically acceptable salt, hydrate, solvate, prodrug, stereoisomer, or tautomer thereof, wherein:
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (BF-II);
wherein no more than 2 of L¹, X¹, X², L², and L³ can simultaneously be a bond;
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2, wherein the Targeting Ligand is a group capable of binding to a Target Protein.

127. The bifunctional compound of any one of the preceding claims, or a pharmaceutically acceptable salt, hydrate, solvate, prodrug, stereoisomer, or tautomer thereof, wherein n is 1.

128. The bifunctional compound of any one of the preceding claims, or a pharmaceutically acceptable salt, hydrate, solvate, prodrug, stereoisomer, or tautomer thereof, wherein n is 2.

129. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

130. The bifunctional compound of any one of the preceding claims, or a pharmaceutically acceptable salt, hydrate, solvate, prodrug, stereoisomer, or tautomer thereof, wherein R^{d3} is H.

131. The bifunctional compound of any one of the preceding claims, wherein the compound has the Formula (BF-III): 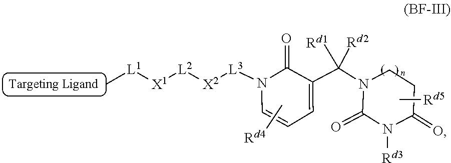
or a pharmaceutically acceptable salt, hydrate, solvate, prodrug, stereoisomer, or tautomer thereof, wherein:
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (BF-III);
wherein no more than 2 of L¹, X¹, X², L², and L³ can simultaneously be a bond;
R′ is hydrogen or C₁₋₆ alkyl;
R¹ and R² are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2, wherein the Targeting Ligand is a group capable of binding to a Target Protein.

132. The bifunctional compound of any one of the preceding claims, or a pharmaceutically acceptable salt, hydrate, solvate, prodrug, stereoisomer, or tautomer thereof, wherein n is 1.

133. The bifunctional compound of any one of the preceding claims, or a pharmaceutically acceptable salt, hydrate, solvate, prodrug, stereoisomer, or tautomer thereof, wherein n is 2.

134. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

135. The bifunctional compound of any one of the preceding claims, or a pharmaceutically acceptable salt, hydrate, solvate, prodrug, stereoisomer, or tautomer thereof, wherein R^{d3} is H.

136. The bifunctional compound of any one of the preceding claims, or a pharmaceutically acceptable salt, hydrate, solvate, prodrug, stereoisomer, or tautomer thereof, wherein —X¹-L²-X²— is:

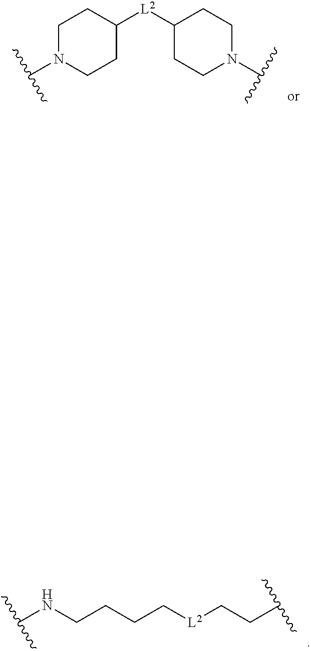

137. The bifunctional compound of any one of the preceding claims, or a pharmaceutically acceptable salt, hydrate, solvate, prodrug, stereoisomer, or tautomer thereof, wherein L¹ is —O— or C₁₋₆ alkylene.

138. The bifunctional compound of any one of the preceding claims, or a pharmaceutically acceptable salt, hydrate, solvate, prodrug, stereoisomer, or tautomer thereof, wherein R^{d1} and R^{d2} are both methyl.

139. The bifunctional compound of any one of the preceding claims, or a pharmaceutically acceptable salt, hydrate, solvate, prodrug, stereoisomer, or tautomer thereof, wherein R^{d1} and R^{d2} are both H.

140. The bifunctional compound of any one of the preceding claims, or a pharmaceutically acceptable salt, hydrate, solvate, prodrug, stereoisomer, or tautomer thereof, wherein R^{d4} is H or C₁₋₃ alkyl.

141. The bifunctional compound of any one of the preceding claims, or a pharmaceutically acceptable salt, hydrate, solvate, prodrug, stereoisomer, or tautomer thereof, wherein R⁵ is H or C₁₋₃ alkyl.

142. The bifunctional compound of any one of the preceding claims, wherein the Targeting Ligase Binder-Linker has Formula (TLB-L-VI): 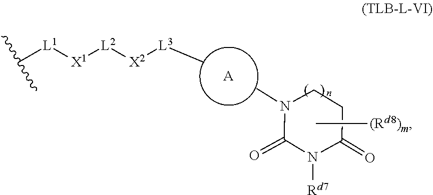
or a pharmaceutically acceptable salt, hydrate, solvate, prodrug, stereoisomer, or tautomer thereof, wherein: [symbol]
denotes the point of attachment to the Targeting Ligand in Formula (I);
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (TLB-L-VI);
wherein no more than 2 of L¹, X¹, X², L², and L³ can simultaneously be a bond;
R′ is hydrogen or C₁₋₆ alkyl;
Ring A is a 6-membered aryl or 6-membered heteroaryl, each of which is independently substituted with 0-4 occurrences of R^{d6};
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{p} is H or C₁₋₆ alkyl;
each R^{d8} is independently selected from the group consisting of H, C₁₋₆ alkyl, halogen, C₁₋₆ haloalkyl, and C₁₋₆ heteroalkyl;
or two R^{d8} together with the carbon atoms to which they are attached form a cycloalkyl;
or two R^{d8} attached to the same carbon atom form a C₃₋₄ spirocycloalkyl;
m is 1 or 2; and
n is 1 or 2.

143. The bifunctional compound of any one of the preceding claims, or a pharmaceutically acceptable salt, hydrate, solvate, prodrug, stereoisomer, or tautomer thereof, wherein n is 1.

144. The bifunctional compound of any one of the preceding claims, or a pharmaceutically acceptable salt, hydrate, solvate, prodrug, stereoisomer, or tautomer thereof, wherein n is 2.

145. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

146. The bifunctional compound of any one of the preceding claims, or a pharmaceutically acceptable salt, hydrate, solvate, prodrug, stereoisomer, or tautomer thereof, wherein R^{d3} is H.

147. The bifunctional compound of any one of the preceding claims, wherein the Targeting Ligase Binder-Linker has Formula (TLB-L-VII): 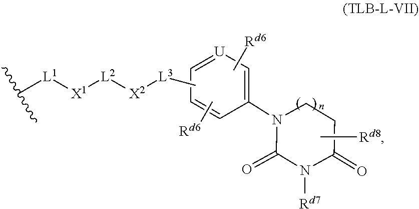
or a pharmaceutically acceptable salt, hydrate, solvate, prodrug, stereoisomer, or tautomer thereof, wherein: [symbol]
denotes the point of attachment to the Targeting Ligand in Formula (I);
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (TLB-L-VII);
wherein no more than 2 of L¹, X¹, X², L², and L³ can simultaneously be a bond;
R′ is hydrogen or C₁₋₆ alkyl;
U is —CR^{d6} or N;
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d8} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

148. The bifunctional compound of any one of the preceding claims, or a pharmaceutically acceptable salt, hydrate, solvate, prodrug, stereoisomer, or tautomer thereof, wherein n is 1.

149. The bifunctional compound of any one of the preceding claims, or a pharmaceutically acceptable salt, hydrate, solvate, prodrug, stereoisomer, or tautomer thereof, wherein n is 2.

150. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

151. The bifunctional compound of any one of the preceding claims, or a pharmaceutically acceptable salt, hydrate, solvate, prodrug, stereoisomer, or tautomer thereof, wherein R^{d3} is H.

152. The bifunctional compound of any one of the preceding claims, or a pharmaceutically acceptable salt, hydrate, solvate, prodrug, stereoisomer, or tautomer thereof, wherein L³ is selected from the group consisting of a bond, —O—, —C(O)—, —S(O)₂—, C₁₋₆ alkylene, C₂₋₆ alkynylene, and C₁₋₆ heteroalkylene.

153. The bifunctional compound of any one of the preceding claims, or a pharmaceutically acceptable salt, hydrate, solvate, prodrug, stereoisomer, or tautomer thereof, wherein one of X¹ and X² is not a bond.

154. The bifunctional compound of any one of the preceding claims, or a pharmaceutically acceptable salt, hydrate, solvate, prodrug, stereoisomer, or tautomer thereof, wherein one of X¹ and X² is a bond, and the other is a carbocyclyl or heterocyclyl.

155. The bifunctional compound of any one of the preceding claims, or a pharmaceutically acceptable salt, hydrate, solvate, prodrug, stereoisomer, or tautomer thereof, wherein one of X¹ and X² is a bond, and the other is a heterocyclyl.

156. The bifunctional compound of any one of the preceding claims, wherein the Targeting Ligase Binder-Linker has Formula (TLB-L-VIII or TLB-L-IX): 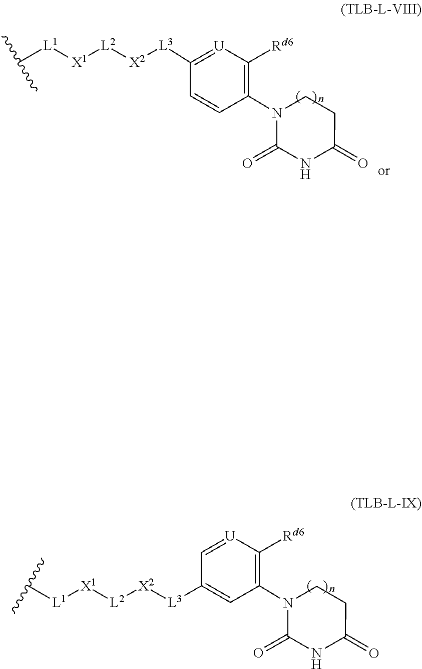
or a pharmaceutically acceptable salt, hydrate, solvate, prodrug, stereoisomer, or tautomer thereof, wherein the point of attachment to the Targeting Ligand is through L¹.

157. The bifunctional compound of any one of the preceding claims, or a pharmaceutically acceptable salt, hydrate, solvate, prodrug, stereoisomer, or tautomer thereof, wherein n is 1.

158. The bifunctional compound of any one of the preceding claims, or a pharmaceutically acceptable salt, hydrate, solvate, prodrug, stereoisomer, or tautomer thereof, wherein n is 2.

159. The bifunctional compound of any one of the preceding claims, wherein the Targeting Ligase Binder-Linker, or a pharmaceutically acceptable salt, hydrate, solvate, prodrug, stereoisomer, or tautomer thereof, has a Formula selected from:

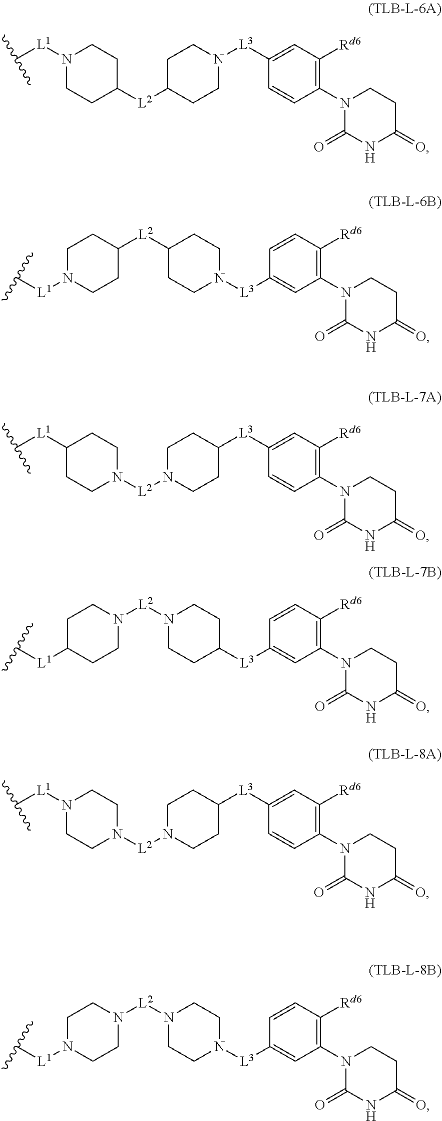

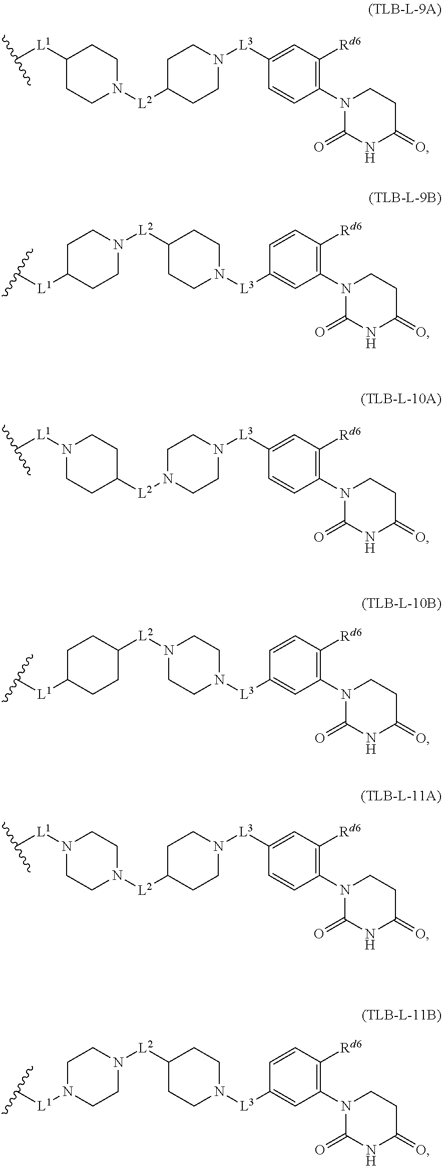

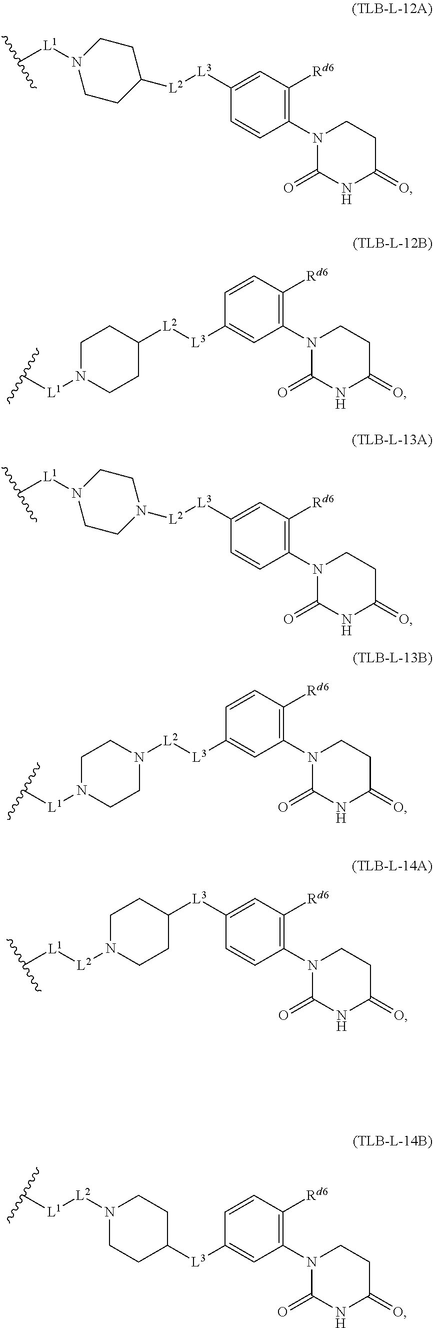

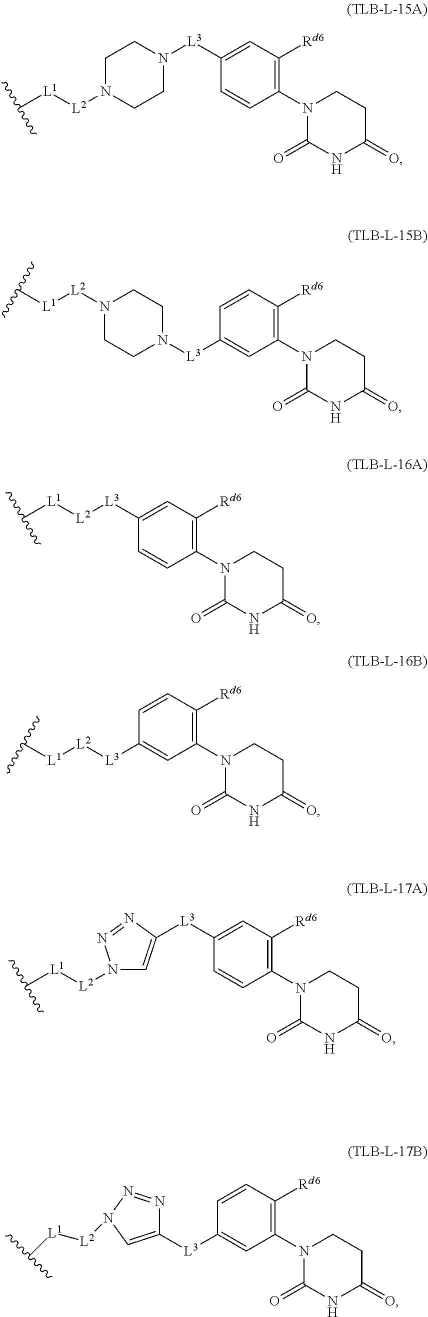

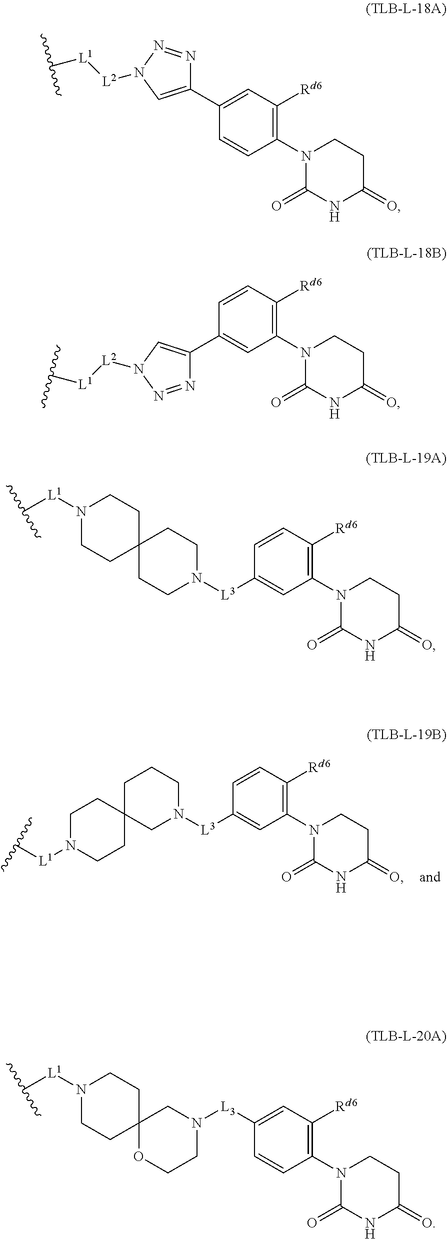

160. The bifunctional compound of any one of the preceding claims, wherein the compound has the Formula (BF-IV): 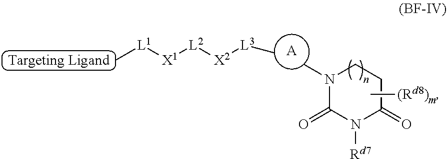
or a pharmaceutically acceptable salt, hydrate, solvate, prodrug, stereoisomer, or tautomer thereof, wherein:
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (BF-IV);
wherein no more than 2 of L¹, X¹, X², L², and L³ can simultaneously be a bond;
R′ is hydrogen or C₁₋₆ alkyl;
Ring A is a 6-membered aryl or 6-membered heteroaryl, each of which is independently substituted with 0-4 occurrences of R^{d6};
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{p} is H or C₁₋₆ alkyl;
each R^{d8} is independently selected from the group consisting of H, C₁₋₆ alkyl, halogen, C₁₋₆ haloalkyl, and C₁₋₆ heteroalkyl;
or two R^{d8} together with the carbon atoms to which they are attached form a cycloalkyl;
or two R^{d8} attached to the same carbon atom form a C₃₋₄ spirocycloalkyl;
m is 1 or 2; and
n is 1 or 2, wherein the Targeting Ligand is a group capable of binding to a Target Protein.

161. The bifunctional compound of any one of the preceding claims, wherein the compound has the Formula (BF-V-A) or (BF-V-B): 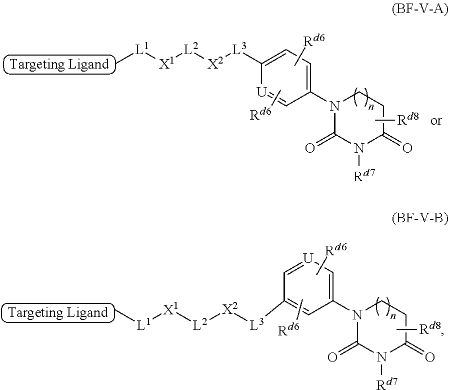
or a pharmaceutically acceptable salt, hydrate, solvate, prodrug, stereoisomer, or tautomer thereof, wherein:
L¹ is selected from the group consisting of a bond, —O—, —NR′, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′, —C(O)—, C₁₋₆ alkylene, C₁₋₆ heteroalkylene, and
*C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (BF-V-A or BF-V-B);
wherein no more than 2 of L¹, X¹, X², L², and L³ can simultaneously be a bond;
R′ is hydrogen or C₁₋₆ alkyl;
U is —CR^{d6} or N;
each R^{d6} is independently selected from the group consisting of H, oxo, C₁₋₆ alkyl, halogen, C₁₋₆ alkoxyl, C₁₋₆ alkoxyalkyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d8} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2, wherein the Targeting Ligand is a group capable of binding to a Target Protein.

162. The bifunctional compound of any one of the preceding claims, or a pharmaceutically acceptable salt, hydrate, solvate, prodrug, stereoisomer, or tautomer thereof, wherein n is 1.

163. The bifunctional compound of any one of the preceding claims, or a pharmaceutically acceptable salt, hydrate, solvate, prodrug, stereoisomer, or tautomer thereof, wherein n is 2.

164. The bifunctional compound of any one of the preceding claims, or a pharmaceutically acceptable salt, hydrate, solvate, prodrug, stereoisomer, or tautomer thereof, wherein R^{d7} is —CH₂OP(O)(OR^{p})₂.

165. The bifunctional compound of any one of the preceding claims, or a pharmaceutically acceptable salt, hydrate, solvate, prodrug, stereoisomer, or tautomer thereof, wherein R^{d7} is H.

166. The bifunctional compound of any one of the preceding claims, or a pharmaceutically acceptable salt, hydrate, solvate, prodrug, stereoisomer, or tautomer thereof, wherein U is —CR^{d6}.

167. The bifunctional compound of any one of the preceding claims, or a pharmaceutically acceptable salt, hydrate, solvate, prodrug, stereoisomer, or tautomer thereof, wherein R^{d8} is H.

168. The bifunctional compound of any one of the preceding claims, or a pharmaceutically acceptable salt, hydrate, solvate, prodrug, stereoisomer, or tautomer thereof, wherein R^{d7} and R^{d8} are each independently H.

169. The bifunctional compound of any one of the preceding claims, or a pharmaceutically acceptable salt, hydrate, solvate, prodrug, stereoisomer, or tautomer thereof, wherein R^{d6} is H.

170. The bifunctional compound of any one of the preceding claims, or a pharmaceutically acceptable salt, hydrate, solvate, prodrug, stereoisomer, or tautomer thereof, wherein R^{d6} is selected from the group consisting of H, halogen, C₁₋₆ alkyl, and C₁₋₆ alkoxyl.

171. The bifunctional compound of any one of the preceding claims, or a pharmaceutically acceptable salt, hydrate, solvate, prodrug, stereoisomer, or tautomer thereof, wherein R^{d6} is selected from the group consisting of H, halogen, C₁₋₆ alkyl, and C₁₋₆ alkoxyl; and R^{d7}, and R^{d8} are each H.

172. The bifunctional compound of any one of the preceding claims, or a pharmaceutically acceptable salt, hydrate, solvate, prodrug, stereoisomer, or tautomer thereof, wherein L¹-X¹-L²-X²-L³ is selected from the group consisting of:

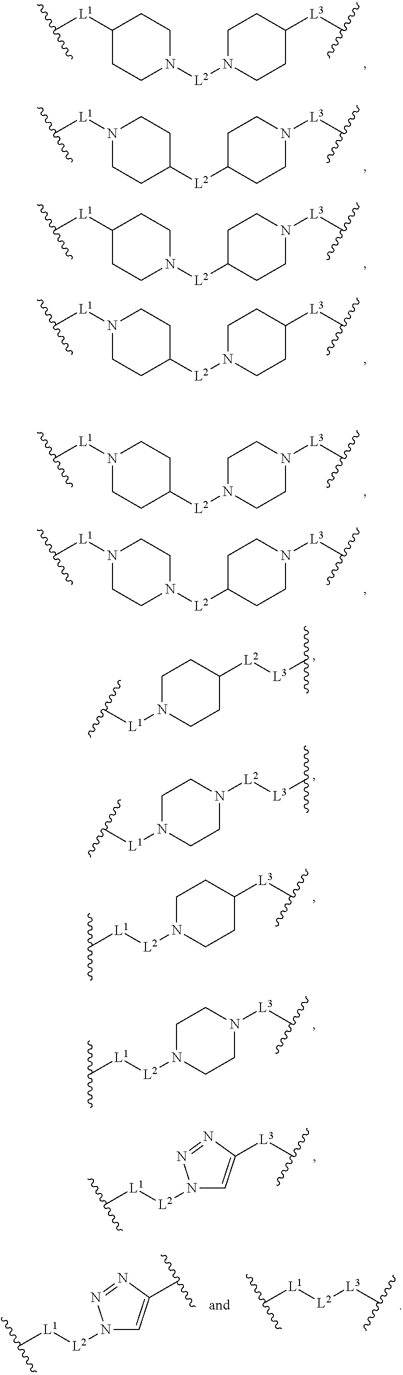

173. The bifunctional compound of any one of the preceding claims, or a pharmaceutically acceptable salt, hydrate, solvate, prodrug, stereoisomer, or tautomer thereof, wherein L³ is selected from the group consisting of a bond, —O—, —C(O)—, —S(O)₂—, C₁₋₆ alkylene, C₂₋₆ alkynylene, and C₁₋₆ heteroalkylene.

174. The bifunctional compound of any one of the preceding claims, or a pharmaceutically acceptable salt, hydrate, solvate, prodrug, stereoisomer, or tautomer thereof, wherein the Target Protein is selected from the group listed in Table 1.

175. The bifunctional compound of any one of the preceding claims, or a pharmaceutically acceptable salt, hydrate, solvate, prodrug, stereoisomer, or tautomer thereof, wherein the Target Protein is selected from the group listed in Table 2.

176. The bifunctional compound of any one of the preceding claims, wherein the Targeting is a BRD9 targeting ligand of Formula (BRD9-I): 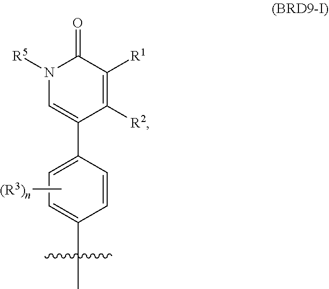
or a pharmaceutically acceptable salt, hydrate, solvate, prodrug, stereoisomer, or tautomer thereof, wherein:
R¹ and R² are independently selected from the group consisting of hydrogen and C₁₋₆ alkyl; or R¹ and R² together with the atoms to which they are attached form an aryl or heteroaryl;
R³ are each independently selected from the group consisting of C₁₋₆ alkyl, C₁₋₆ alkoxyl, and halogen;
R⁵ is selected from the group consisting of hydrogen and C₁₋₃ alkyl;
n is 0, 1, or 2.

177. The bifunctional compound of any one of the preceding claims, wherein the Targeting Ligand is a BTK targeting ligand of Formula (BTK-I): 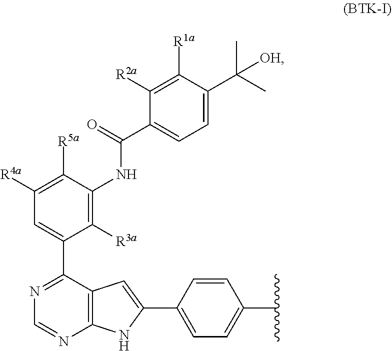
or a pharmaceutically acceptable salt, hydrate, solvate, prodrug, stereoisomer, or tautomer thereof, wherein:
R^{1a} is H or halo;
R^{2a} is halo;
R^{3a} is C₁₋₆ alkyl;
R^{4a} is halo; and
R^{5a} is H or halo.

178. A pharmaceutical composition comprising a compound of any one of the preceding claims, or a pharmaceutically acceptable salt, hydrate, solvate, prodrug, stereoisomer, or tautomer thereof, and a pharmaceutically acceptable carrier.

179. A pharmaceutical combination comprising a compound of any one of the preceding claims, or a pharmaceutically acceptable salt, hydrate, solvate, prodrug, stereoisomer, or tautomer thereof, and one or more additional therapeutic agent(s).

180. A method for inducing degradation of a Target Protein in a subject in need thereof, the method comprising administering to the subject a therapeutically effective amount of a compound of any one of the preceding claims, or a pharmaceutically acceptable salt, hydrate, solvate, prodrug, stereoisomer, or tautomer thereof.

181. A method of inhibiting, reducing, or eliminating the activity of a Target Protein, the method comprising administering to the subject a compound of any one of the preceding claims, or a pharmaceutically acceptable salt, hydrate, solvate, prodrug, stereoisomer, or tautomer thereof.

182. The method of any one of the preceding claims, wherein inhibiting, reducing, or eliminating the activity of a Target Protein comprises recruiting a ligase (e.g., Cereblon E3 Ubiquitin ligase) with the Targeting Ligase Binder, e.g., a Targeting Ligase Binder described herein, of the bifunctional compound, e.g., a bifunctional compound described herein, forming a ternary complex of the Target Protein, bifunctional compound, and the ligase, to thereby inhibit, reduce or eliminate the activity of the Target Protein.

183. The method of any one of the preceding claims, wherein the Target Protein is selected from the group listed in Table 1 or Table 2.

184. A method of treating a Target Protein-mediated disorder, disease, or condition in a patient comprising administering to the patient the compound of any one of the preceding claims, or a pharmaceutically acceptable salt, hydrate, solvate, prodrug, stereoisomer, or tautomer thereof.

185. The method of any one of the preceding claims, wherein the disorder is selected from a respiratory disorder, a proliferative disorder, an autoimmune disorder, an autoinflammatory disorder, an inflammatory disorder, a neurological disorder, and an infectious disease or disorder.

186. The method of any one of the preceding claims, wherein the disorder is a proliferative disorder.

187. The method of any one of the preceding claims, wherein the proliferative disorder is cancer.

188. A method of treating cancer in a subject in need thereof, the method comprising administering to the subject a therapeutically effective amount of a compound of any one of the preceding claims, or a pharmaceutically acceptable salt thereof.

189. A compound of Formula (ILB-I): 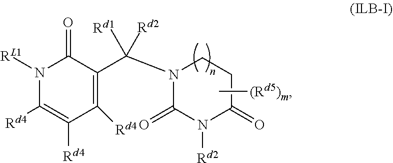
or a pharmaceutically acceptable salt, hydrate, solvate, prodrug, stereoisomer, or tautomer thereof, wherein:
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, C₁₋₆ alkyl, halogen, C₁₋₆ haloalkyl, C₁₋₆ alkoxyl; C₁₋₆ alkoxyalkyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
or two R^{d5} together with the carbon atoms to which they are attached form a cycloalkyl;
or two R^{d5} attached to the same carbon atom form a C₃₋₄ spirocycloalkyl;
R^{L1} is selected from the group consisting of C₁₋₆ alkyl, C₂₋₆ alkenyl, C₂₋₆ alkynyl, C₂₋₆ hydroxyalkyl, —(CH₂)₂₋₆NHR^{c}, C₃₋₆ heteroalkyl, C₂₋₆ haloalkyl, —(CH₂)₁₋₃C(O)OH, —(CH₂)₁₋₃C(O)H, —(CH₂)₁₋₃O(CH₂)₁₋₃C(O)H, —(CH₂)₀₋₃C₃₋₇ carbocyclyl, —(CH₂)₀₋₃ heterocyclyl, C₆ aryl, and heteroaryl, wherein the carbocyclyl, heterocyclyl, aryl, and heteroaryl is substituted with 0-2 occurrences of —O-heterocyclyl, —O-carbocyclyl, —C(O)— heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, or C₁₋₆ heteroalkyl;
R^{p} is H or C₁₋₆ alkyl;
m is 1 or 2; and
n is 1 or 2.

190. The bifunctional compound of any one of the preceding claims, or a pharmaceutically acceptable salt, hydrate, solvate, prodrug, stereoisomer, or tautomer thereof, wherein n is 1.

191. The bifunctional compound of any one of the preceding claims, or a pharmaceutically acceptable salt, hydrate, solvate, prodrug, stereoisomer, or tautomer thereof, wherein n is 2.

192. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

193. The bifunctional compound of any one of the preceding claims, or a pharmaceutically acceptable salt, hydrate, solvate, prodrug, stereoisomer, or tautomer thereof, wherein R^{d3} is H.

194. The compound of any one of the preceding claims, or a pharmaceutically acceptable salt, hydrate, solvate, prodrug, stereoisomer, or tautomer thereof, selected from:

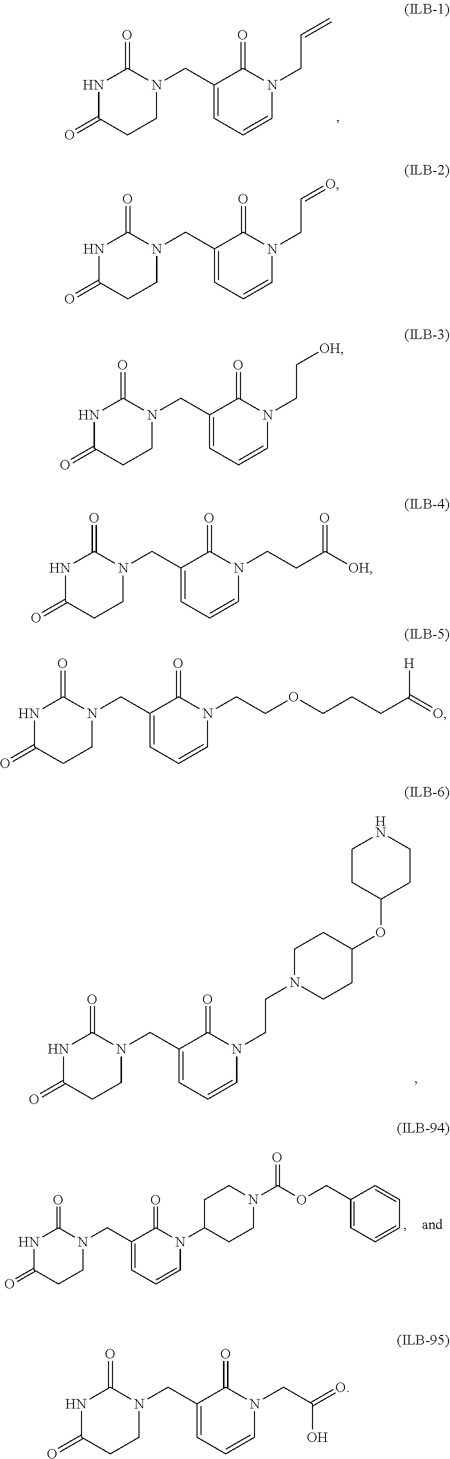

195. A compound of Formula (ILB-II): 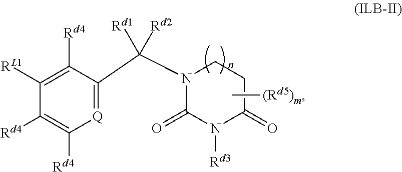
or a pharmaceutically acceptable salt, hydrate, solvate, prodrug, stereoisomer, or tautomer thereof, wherein:
Q is N or CR^{d4};
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂(O)(CH₂)₂Si(CH₃)₃, —CH₂OC(O)R^{p}, —CH₂OP(O)OHOR^{p}, —CH₂OP(O)(R^{p})₂, and —CH₂OP(O)(OR^{p})₂;
each R^{d4} is independently selected from the group consisting of H, oxo, C₁₋₆ alkyl, halogen, C₁₋₆ haloalkyl, C₁₋₆ alkoxyl; C₁₋₆ alkoxyalkyl, and C₁₋₆ heteroalkyl;
each R^{d5} is independently selected from the group consisting of H, C₁₋₆ alkyl, halogen, C₁₋₆ haloalkyl, C₁₋₆ heteroalkyl, and C₃₋₆ cycloalkyl;
or two R^{d5} together with the carbon atoms to which they are attached form a cycloalkyl;
or two R^{d5} attached to the same carbon atom form a C₃₋₄ spirocycloalkyl;
R^{L1} is selected from the group consisting of C₂₋₆ alkenyl, C₂₋₆ alkynyl, C₂₋₆ hydroxyalkyl, —(CH₂)₂₋₆NHR^{c}, C₃₋₆ heteroalkyl, C₂₋₆ haloalkyl, —(CH₂)₀₋₃C(O)OH, —(CH₂)₀₋₃C(O)H, —(CH₂)₀₋₃C₃₋₇ carbocyclyl, —(CH₂)₀₋₃ heterocyclyl, C₆ aryl, and heteroaryl, wherein the carbocyclyl, heterocyclyl, aryl, and heteroaryl is substituted with 0-2 occurrences of —O-heterocyclyl, —O-carbocyclyl, —C(O)-heterocyclyl, —C(O)— carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, or C₁₋₆ heteroalkyl;
R^{p} is H or C₁₋₆ alkyl;
m is 1 or 2; and
n is 1 or 2.

196. The bifunctional compound of any one of the preceding claims, or a pharmaceutically acceptable salt, hydrate, solvate, prodrug, stereoisomer, or tautomer thereof, wherein n is 1.

197. The bifunctional compound of any one of the preceding claims, or a pharmaceutically acceptable salt, hydrate, solvate, prodrug, stereoisomer, or tautomer thereof, wherein n is 2.

198. The bifunctional compound of any one of the preceding claims, or a pharmaceutically acceptable salt, hydrate, solvate, prodrug, stereoisomer, or tautomer thereof, wherein R^{d3} is —CH₂OP(O)(OR^{p})₂.

199. The bifunctional compound of any one of the preceding claims, or a pharmaceutically acceptable salt, hydrate, solvate, prodrug, stereoisomer, or tautomer thereof, wherein R^{d3} is H.

200. The compound of any one of the preceding claims, or a pharmaceutically acceptable salt, hydrate, solvate, prodrug, stereoisomer, or tautomer thereof, selected from:

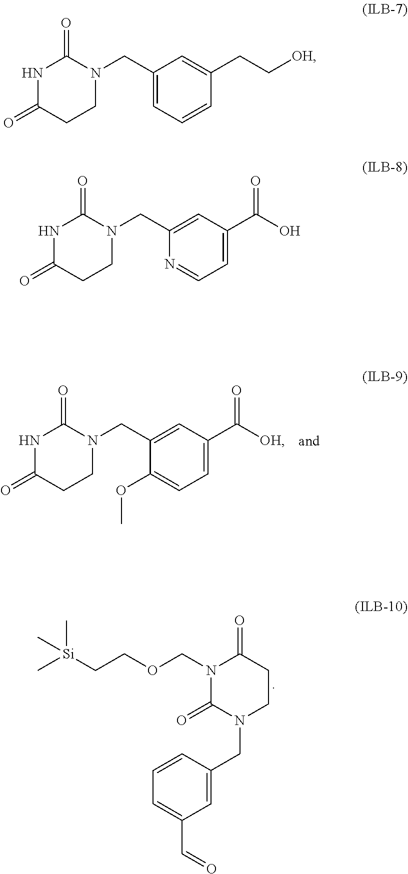 [SMILES: O=C1CCN(Cc2cccc(CCO)c2)C(=O)N1 ; O=C1CCN(Cc2cc(C(=O)O)ccn2)C(=O)N1 ; COc1ccc(C(=O)O)cc1CN1CCC(=O)NC1=O ; C[Si](C)(C)CCOCN1C(=O)CCN(Cc2cccc(C=O)c2)C1=O]

201. A compound of Formula (ILB-III): 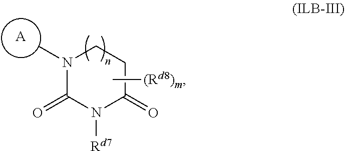
or a pharmaceutically acceptable salt, hydrate, solvate, prodrug, stereoisomer, or tautomer thereof, wherein:
Ring A is selected from the group consisting of: 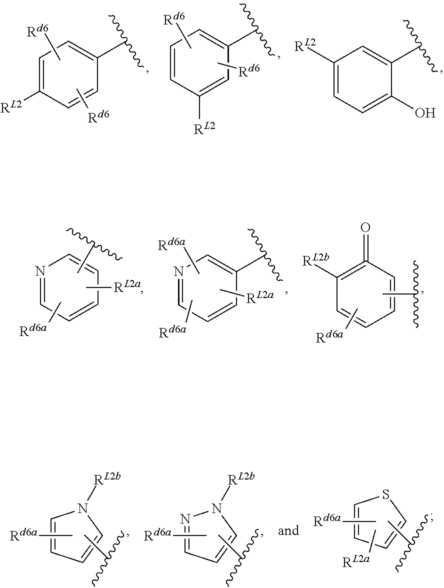 [symbol]
denotes the point of attachment to the base molecule of (ILB-III);
each R^{d6} is independently selected from the group consisting of H, oxo, polyethylene glycol (PEG), halogen, C₁₋₃ alkyl, C₁₋₃ alkoxyl, C₁₋₆ haloalkyl, C₁₋₆ heteroalkyl, and —OC₁₋₇ heteroalkyl;
each R^{d6a} is independently selected from the group consisting of H, hydroxyl, oxo, polyethylene glycol (PEG), halogen, C₁₋₃ alkyl, C₁₋₃ alkoxyl, C₁₋₆ haloalkyl, C₁₋₆ heteroalkyl, and —OC₁₋₇ heteroalkyl;
R^{d7} is H, —CH₂OC(O)R^{p}, —CH₂OP(O)OHOR^{p}, —CH₂OP(O)(R^{p})₂, and —CH₂OP(O)(OR^{p})₂;
each R^{d8} is independently selected from the group consisting of H, C₁₋₆ alkyl, C₁₋₆ haloalkyl, and C₁₋₆ heteroalkyl;
or two R^{d8} together with the carbon atoms to which they are attached form a cycloalkyl;
or two R^{d8} attached to the same carbon atom form a C₃₋₄ spirocycloalkyl;
R^{L2} is selected from the group consisting of hydroxyl, C₂₋₆ alkenyl, C₂₋₆ alkynyl, C₂₋₆ hydroxyalkyl, —(CH₂)₂₋₆NHR^{c}, —(CH₂)₂₋₆NR^{c}R^{d}, —O—(CH₂)₂₋₆NHR^{c}, C₄₋₈ heteroalkyl, C₂₋₆ haloalkyl, —SO₂—NH—(CH₂)₂₋₆NHR^{c}, —(CH₂)₀₋₃C(O)OH, —(CH₂)₀₋₃C(O)OR^{c}, —O—C₂₋₆ alkenyl, —O—(CH₂)₀₋₃C(O)H, —(CH₂)₀₋₃C(O)H, —O—(CH₂)₁₋₃C(O)OH, —(CH₂)₀₋₃ heterocyclyl, —C(O)—(CH₂)₀₋₃ heterocyclyl, —O—(CH₂)₀₋₃ heterocyclyl, —O—(CH₂)₀₋₃C(O)-heterocyclyl, —C₂₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, —(CH₂)₂₋₆N(R^{c})₂, heterocyclyl, heteroaryl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl, wherein the heterocyclyl and heteroaryl is substituted with 0-2 occurrences of halogen;
R^{L2a} is selected from the group consisting of H, hydroxyl, C₂₋₆ alkenyl, C₂₋₆ alkynyl, C₂₋₆ hydroxyalkyl, —(CH₂)₂₋₆NHR^{c}, —(CH₂)₂₋₆NR^{c}R^{d}, —O—(CH₂)₂₋₆NHR^{c}, C₁₋₈ heteroalkyl, C₁₋₆ haloalkyl, —SO₂—NH—(CH₂)₂₋₆NHR^{c}, —(CH₂)₀₋₃C(O)OH, —(CH₂)₀₋₃C(O)OR^{c}, —O—C₂₋₆ alkenyl, —O—(CH₂)₀₋₃C(O)H, —(CH₂)₀₋₃C(O)H, —O—(CH₂)₁₋₃C(O)OH, —(CH₂)₀₋₃ heterocyclyl, —C(O)—(CH₂)₀₋₃ heterocyclyl, —O—(CH₂)₀₋₃ heterocyclyl, —O—(CH₂)₀₋₃C(O)-heterocyclyl, —C₂₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, —(CH₂)₂₋₆N(R^{c})₂, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl, wherein the heterocyclyl is substituted with 0-2 occurrences of halogen;
R^{L2b} is selected from the group consisting of H, polyethylene glycol (PEG), C₁₋₃ alkyl, C₃₋₆ cycloalkyl, C₃₋₆ alkenyl, C₃₋₆ alkynyl, C₂₋₆ hydroxyalkyl, —(CH₂)₂₋₆NHR^{c}, —(CH₂)₂₋₆NR^{c}R^{d}, C₂₋₈ heteroalkyl, C₂₋₆ haloalkyl, —(CH₂)₁₋₃C(O)OH, —(CH₂)₁₋₃C(O)H, —(CH₂)₀₋₃ heterocyclyl, —C(O)—(CH₂)₀₋₃ heterocyclyl, —C₃₋₆ alkynyl-heterocyclyl, and heteroaryl, wherein the alkynyl, heterocyclyl, heteroalkyl, carbocyclyl, and heteroaryl is substituted with 0-2 occurrences of halogen, hydroxyl, —(CH₂)₀₋₃C(O)H, —(CH₂)₂₋₆NHR^{c}, heterocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, C₁₋₆ heteroalkyl, and —C(O)OC₁₋₆ alkyl;
R^{d} is H or C₁₋₄ alkyl; or R^{c} and R^{d} together with the nitrogen atom to which they are attached form a heterocyclyl substituted with 0-2 occurrences of —O-heterocyclyl,
R^{p} is H or C₁₋₆ alkyl;
m is 1 or 2; and
n is 1 or 2.

202. The bifunctional compound of any one of the preceding claims, or a pharmaceutically acceptable salt, hydrate, solvate, prodrug, stereoisomer, or tautomer thereof, wherein ring A is selected from the group consisting of:

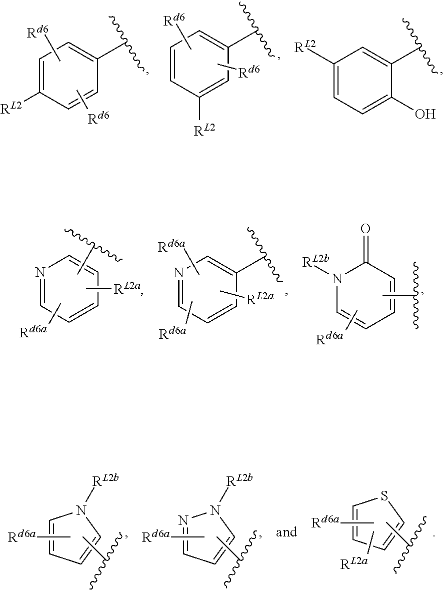

203. The bifunctional compound of any one of the preceding claims, or a pharmaceutically acceptable salt, hydrate, solvate, prodrug, stereoisomer, or tautomer thereof, wherein n is 1.

204. The bifunctional compound of any one of the preceding claims, or a pharmaceutically acceptable salt, hydrate, solvate, prodrug, stereoisomer, or tautomer thereof, wherein n is 2.

205. The bifunctional compound of any one of the preceding claims, or a pharmaceutically acceptable salt, hydrate, solvate, prodrug, stereoisomer, or tautomer thereof, wherein R^{d7} is —CH₂OP(O)(OR^{p})₂.

206. The bifunctional compound of any one of the preceding claims, or a pharmaceutically acceptable salt, hydrate, solvate, prodrug, stereoisomer, or tautomer thereof, wherein R^{d7} is H.

207. The compound of any one of the preceding claims, or a pharmaceutically acceptable salt, hydrate, solvate, prodrug, stereoisomer, or tautomer thereof, selected from:

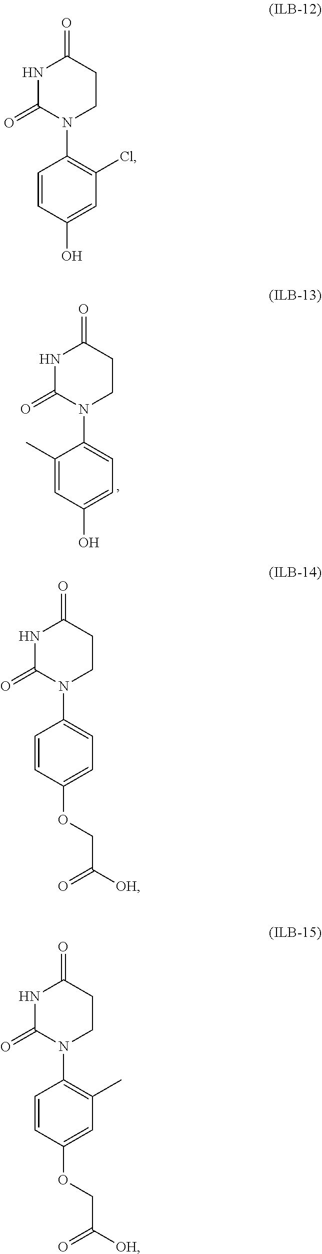 [SMILES: O=C1CCN(c2ccc(O)cc2Cl)C(=O)N1 ; Cc1cc(O)ccc1N1CCC(=O)NC1=O ; O=C(O)COc1ccc(N2CCC(=O)NC2=O)cc1 ; Cc1cc(OCC(=O)O)ccc1N1CCC(=O)NC1=O]

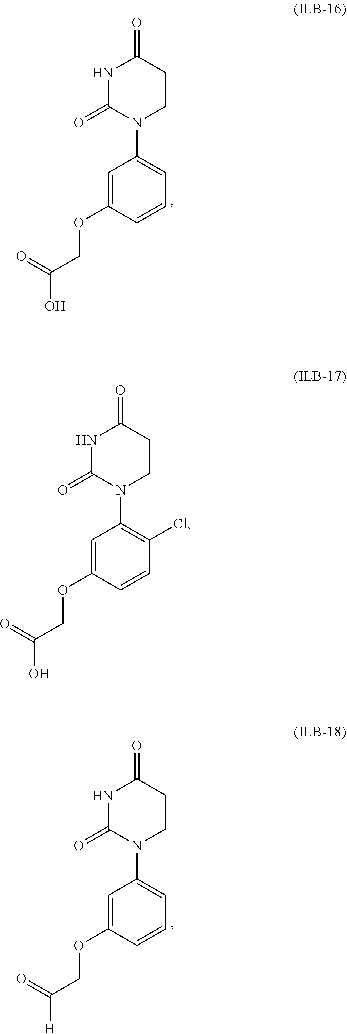 [SMILES: O=C(O)COc1cccc(N2CCC(=O)NC2=O)c1 ; O=C(O)COc1ccc(Cl)c(N2CCC(=O)NC2=O)c1 ; O=CCOc1cccc(N2CCC(=O)NC2=O)c1]

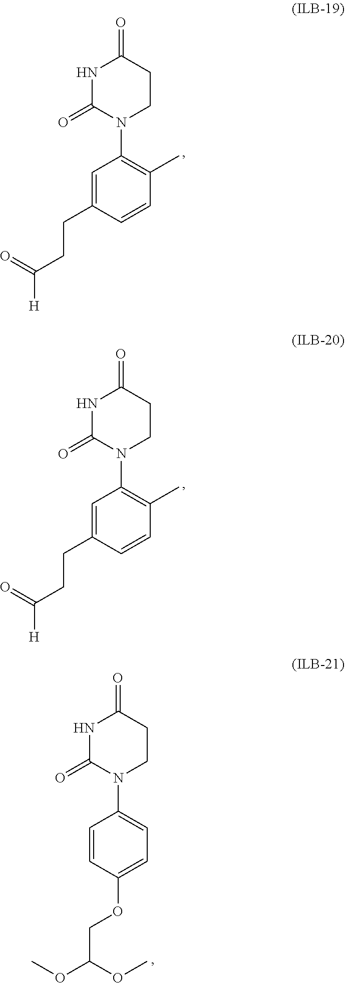

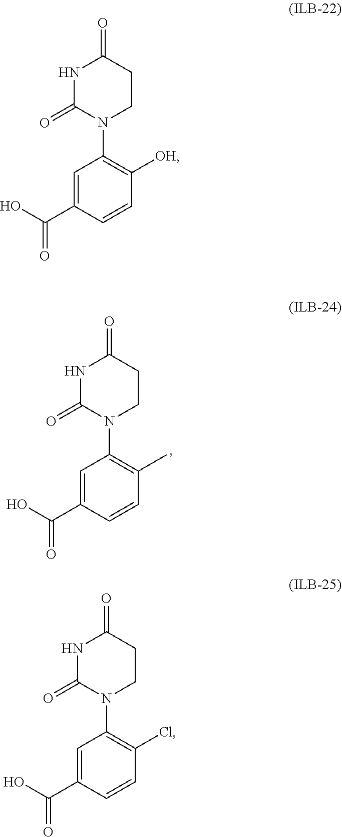 [SMILES: O=C1CCN(c2cc(C(=O)O)ccc2O)C(=O)N1 ; Cc1ccc(C(=O)O)cc1N1CCC(=O)NC1=O ; O=C1CCN(c2cc(C(=O)O)ccc2Cl)C(=O)N1]

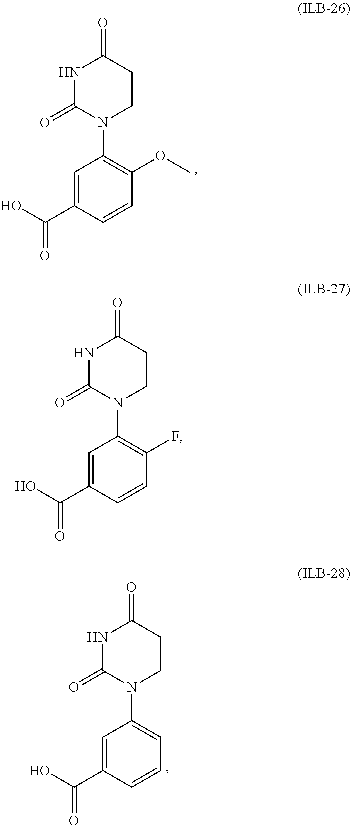 [SMILES: COc1ccc(C(=O)O)cc1N1CCC(=O)NC1=O ; O=C1CCN(c2cc(C(=O)O)ccc2F)C(=O)N1 ; O=C1CCN(c2cccc(C(=O)O)c2)C(=O)N1]

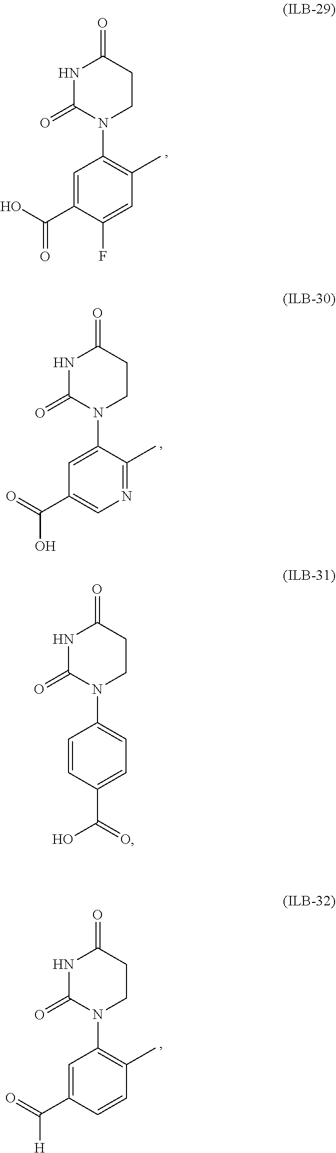 [SMILES: Cc1cc(F)c(C(=O)O)cc1N1CCC(=O)NC1=O ; Cc1ncc(C(=O)O)cc1N1CCC(=O)NC1=O ; O=C1CCN(c2ccc(C(=O)O)cc2)C(=O)N1 ; Cc1ccc(C=O)cc1N1CCC(=O)NC1=O]

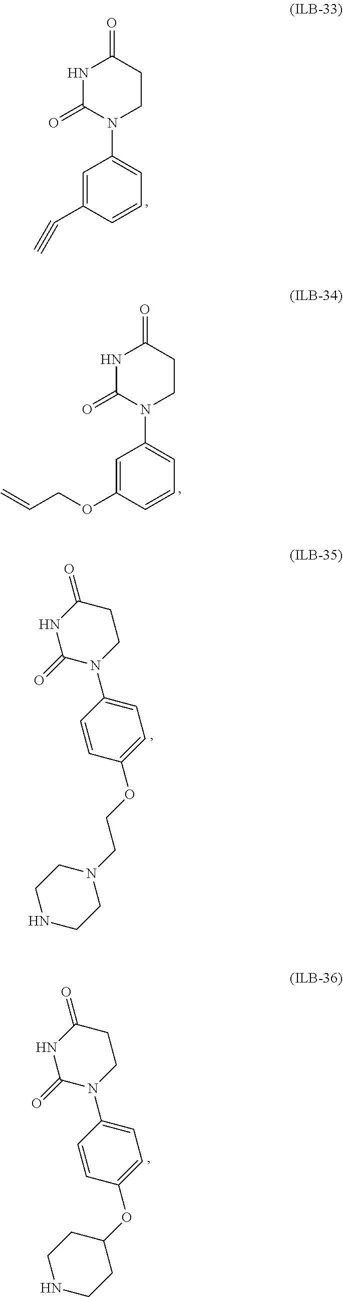 [SMILES: C#Cc1cccc(N2CCC(=O)NC2=O)c1 ; C=CCOc1cccc(N2CCC(=O)NC2=O)c1 ; O=C1CCN(c2ccc(OCCN3CCNCC3)cc2)C(=O)N1 ; O=C1CCN(c2ccc(OC3CCNCC3)cc2)C(=O)N1]

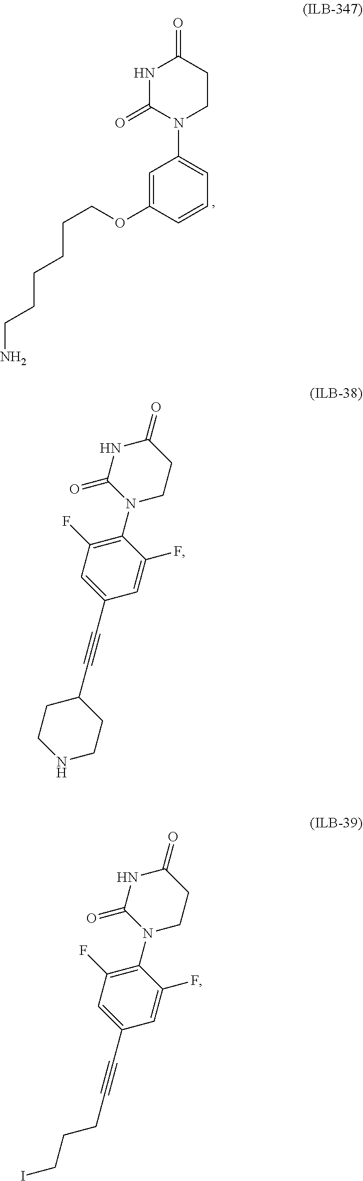 [SMILES: NCCCCCCOc1cccc(N2CCC(=O)NC2=O)c1 ; O=C1CCN(c2c(F)cc(C#CC3CCNCC3)cc2F)C(=O)N1 ; O=C1CCN(c2c(F)cc(C#CCCCI)cc2F)C(=O)N1]

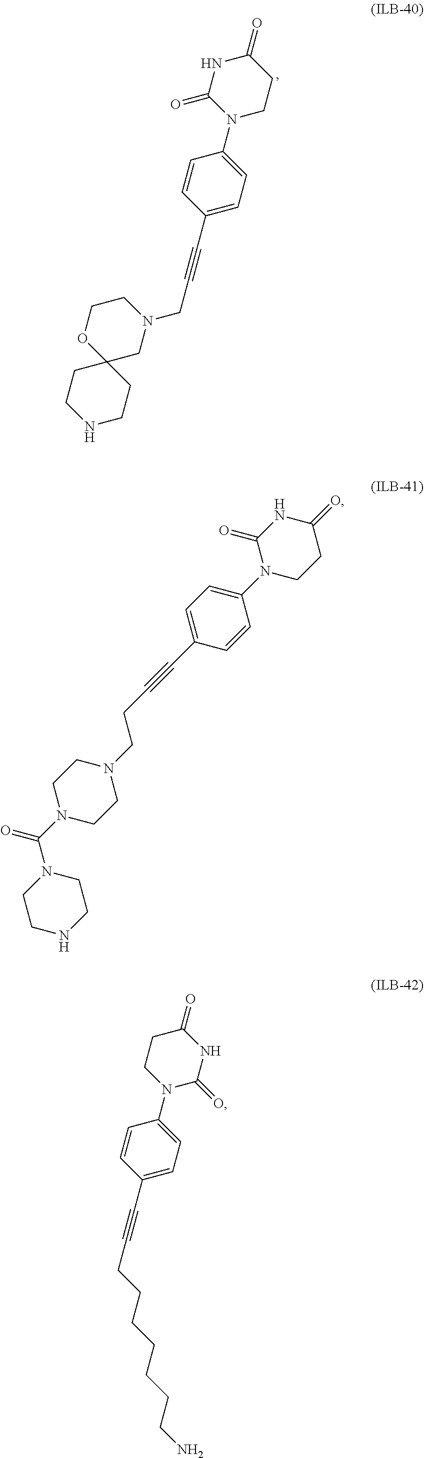 [SMILES: O=C1CCN(c2ccc(C#CCN3CCOC4(CCNCC4)C3)cc2)C(=O)N1 ; O=C1CCN(c2ccc(C#CCCN3CCN(C(=O)N4CCNCC4)CC3)cc2)C(=O)N1 ; NCCCCCCCC#Cc1ccc(N2CCC(=O)NC2=O)cc1]

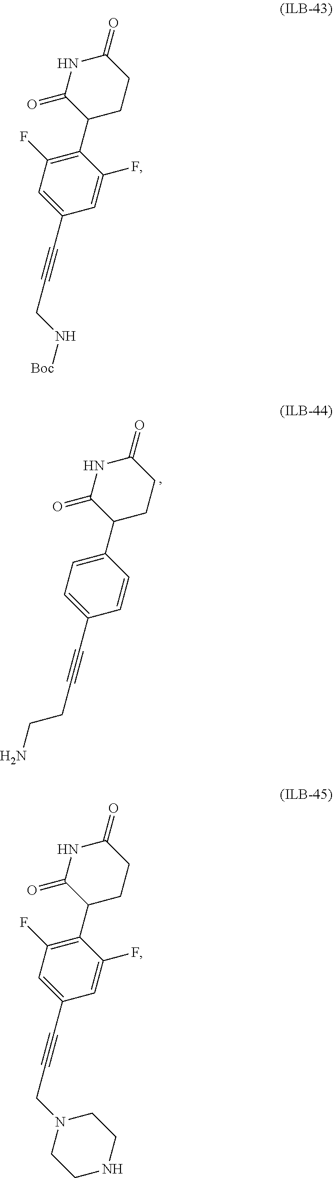 [SMILES: CC(C)(C)OC(=O)NCC#Cc1cc(F)c(C2CCC(=O)NC2=O)c(F)c1 ; NCCC#Cc1ccc(C2CCC(=O)NC2=O)cc1 ; O=C1CCC(c2c(F)cc(C#CCN3CCNCC3)cc2F)C(=O)N1]

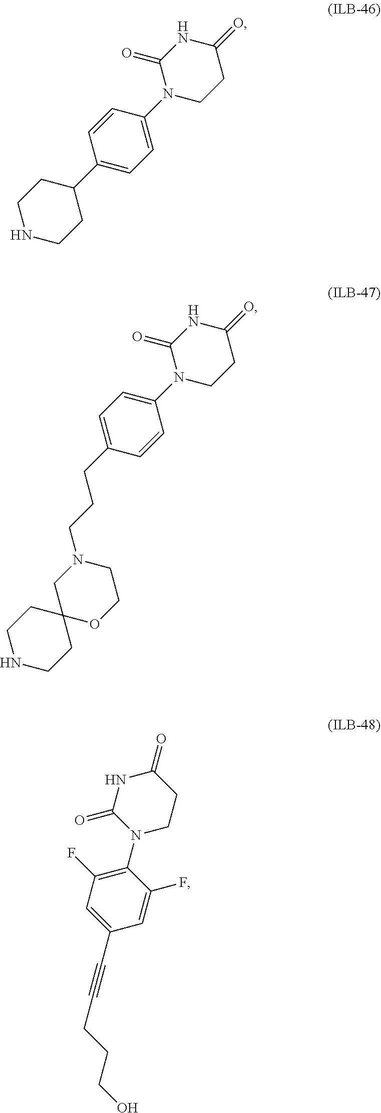 [SMILES: O=C1CCN(c2ccc(C3CCNCC3)cc2)C(=O)N1 ; O=C1CCN(c2ccc(CCCN3CCOC4(CCNCC4)C3)cc2)C(=O)N1 ; O=C1CCN(c2c(F)cc(C#CCCCO)cc2F)C(=O)N1]

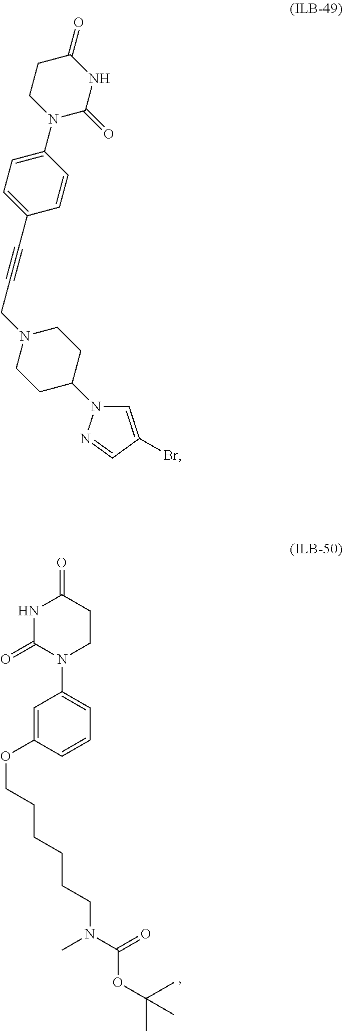 [SMILES: O=C1CCN(c2ccc(C#CCN3CCC(n4cc(Br)cn4)CC3)cc2)C(=O)N1 ; CN(CCCCCCOc1cccc(N2CCC(=O)NC2=O)c1)C(=O)OC(C)(C)C]

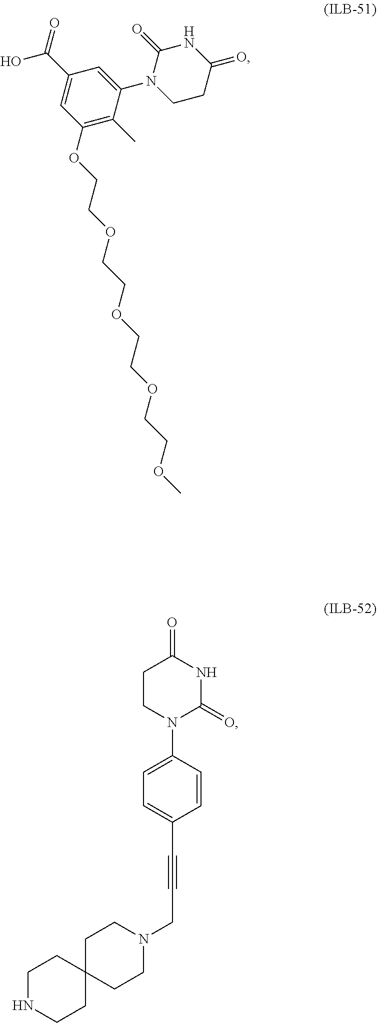 [SMILES: COCCOCCOCCOCCOc1cc(C(=O)O)cc(N2CCC(=O)NC2=O)c1C ; O=C1CCN(c2ccc(C#CCN3CCC4(CCNCC4)CC3)cc2)C(=O)N1]

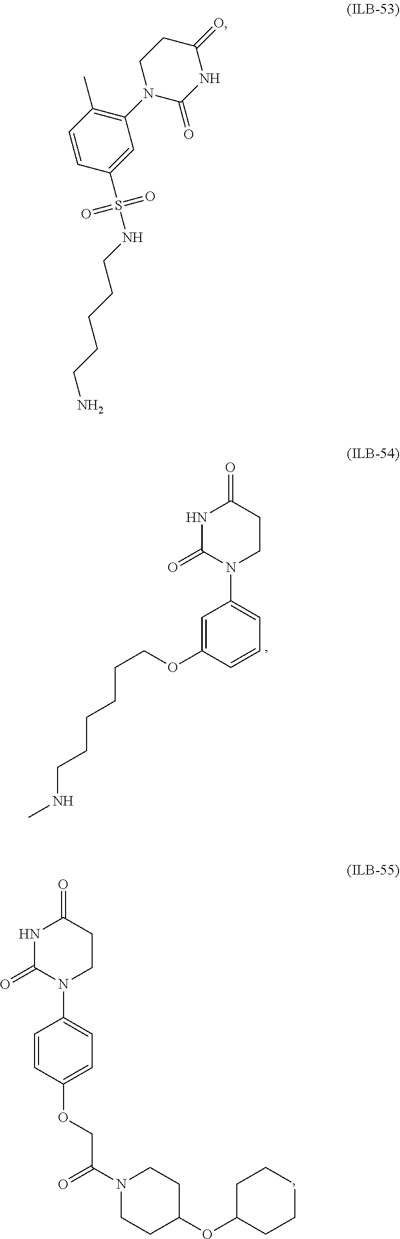

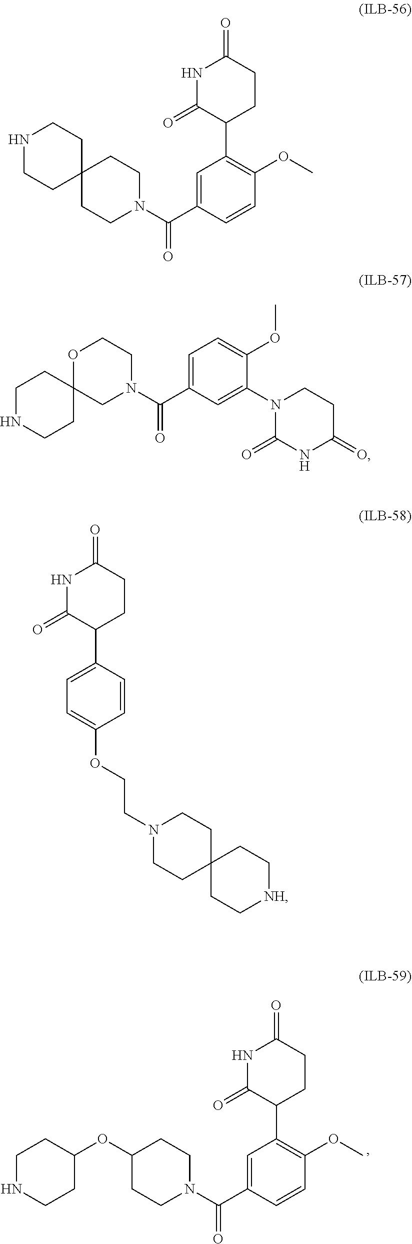 [SMILES: COc1ccc(C(=O)N2CCC3(CCNCC3)CC2)cc1C1CCC(=O)NC1=O ; COc1ccc(C(=O)N2CCOC3(CCNCC3)C2)cc1N1CCC(=O)NC1=O ; O=C1CCC(c2ccc(OCCN3CCC4(CCNCC4)CC3)cc2)C(=O)N1 ; COc1ccc(C(=O)N2CCC(OC3CCNCC3)CC2)cc1C1CCC(=O)NC1=O]

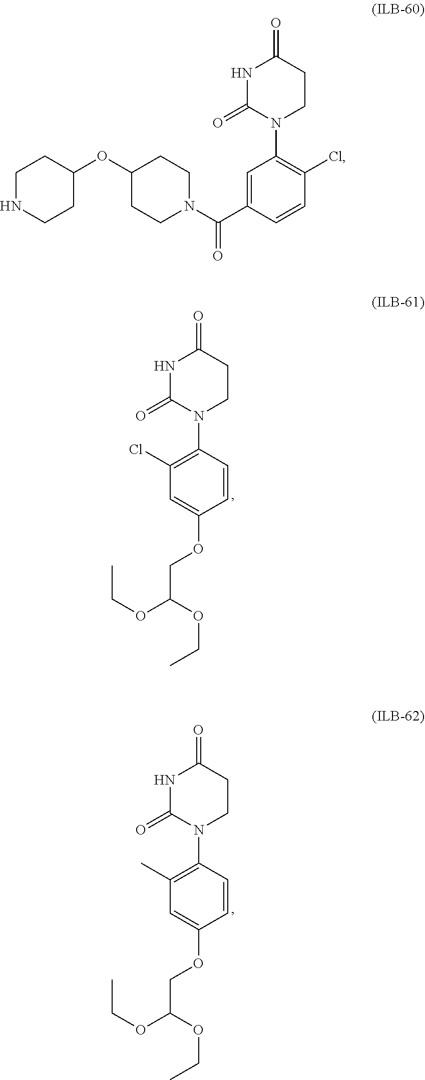 [SMILES: O=C1CCN(c2cc(C(=O)N3CCC(OC4CCNCC4)CC3)ccc2Cl)C(=O)N1 ; CCOC(COc1ccc(N2CCC(=O)NC2=O)c(Cl)c1)OCC ; CCOC(COc1ccc(N2CCC(=O)NC2=O)c(C)c1)OCC]

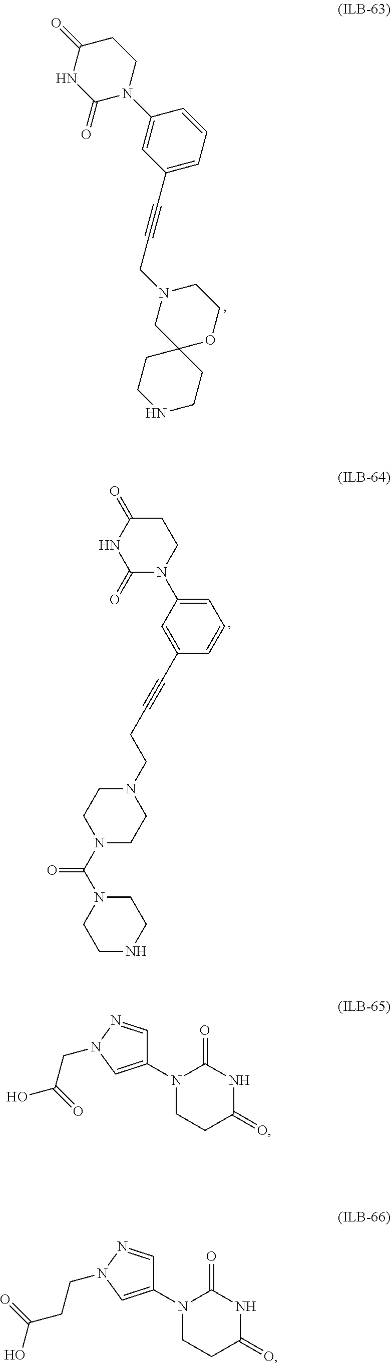 [SMILES: O=C1CCN(c2cccc(C#CCN3CCOC4(CCNCC4)C3)c2)C(=O)N1 ; O=C1CCN(c2cccc(C#CCCN3CCN(C(=O)N4CCNCC4)CC3)c2)C(=O)N1 ; O=C(O)Cn1cc(N2CCC(=O)NC2=O)cn1 ; O=C(O)CCn1cc(N2CCC(=O)NC2=O)cn1]

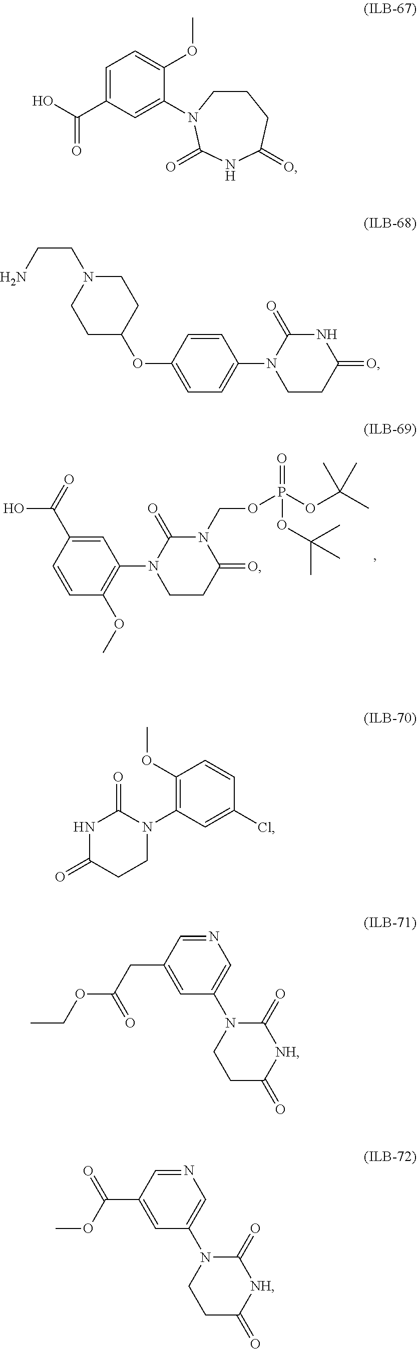

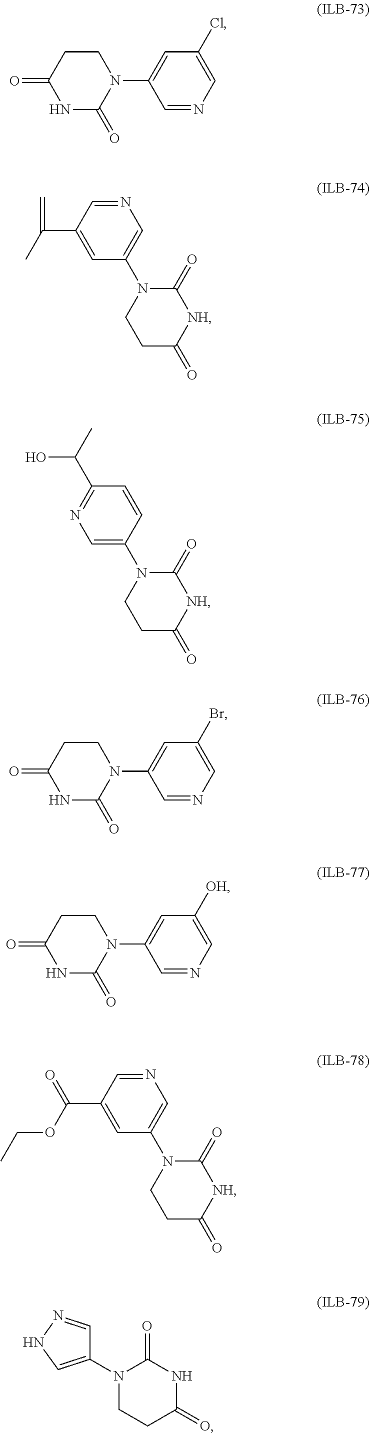

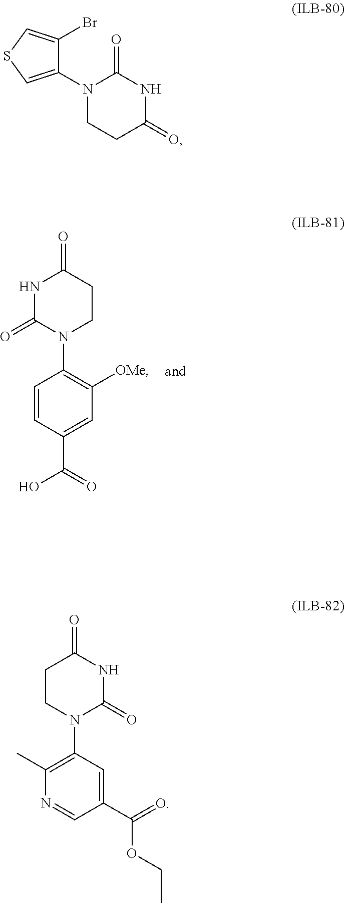 [SMILES: O=C1CCN(c2cscc2Br)C(=O)N1 ; COc1cc(C(=O)O)ccc1N1CCC(=O)NC1=O ; CCOC(=O)c1cnc(C)c(N2CCC(=O)NC2=O)c1]

208. A compound of Formula (ILB-IV): 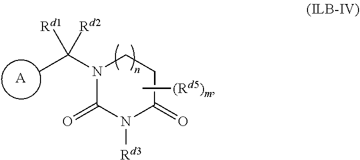
or a pharmaceutically acceptable salt, hydrate, solvate, prodrug, stereoisomer, or tautomer thereof, wherein:
Ring A is selected from the group consisting of 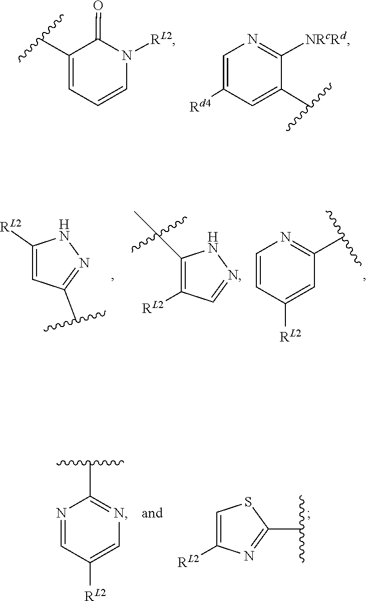 [symbol]
denotes the point of attachment to the base molecule of (ILB-IV);
R^{d1} and R^{d2} are each independently selected from the group consisting of H, C₁₋₆ alkyl, C₁₋₆ haloalkyl, C₁₋₆ heteroalkyl, and C₃₋₆ cycloalkyl;
R^{d3} is H, —CH₂OC(O)R^{P}, —CH₂OP(O)OHOR^{P}, —CH₂OP(O)(R^{P})₂, and —CH₂OP(O)(OR^{p})₂;
R^{d4} is selected from the group consisting of H, hydroxyl, oxo, polyethylene glycol (PEG), halogen, C₁₋₃ alkyl, C₃₋₆ cycloalkyl, C₁₋₃ alkoxyl, C₁₋₆ haloalkyl, C₁₋₆ heteroalkyl, and —OC₁₋₇ heteroalkyl;
each R^{d5} is independently selected from the group consisting of H, C₁₋₆ alkyl, C₁₋₆ haloalkyl, and C₁₋₆ heteroalkyl;
or two R^{d8} together with the carbon atoms to which they are attached form a cycloalkyl;
or two R^{d8} attached to the same carbon atom form a C₃₋₄ spirocycloalkyl;
R^{L2} is selected from the group consisting of hydroxyl, halogen, C₂₋₆ alkyl, C₁₋₃ alkoxyl, C₂₋₆ alkenyl, C₂₋₆ alkynyl, C₂₋₆ hydroxyalkyl, —(CH₂)₂₋₆NHR^{c}, —(CH₂)₀₋₆NR^{c}R^{d}, —O—(CH₂)₂₋₆NHR^{c}, C₃₋₈ heteroalkyl, C₁₋₆ haloalkyl, —SO₂—NH—(CH₂)₂₋₆NHR^{c}, —(CH₂)₀₋₃C(O)OH, —O—(CH₂)₁₋₃C(O)H, —(CH₂)₁₋₃C(O)H, —O—(CH₂)₁₋₃C(O)OH, —(CH₂)₀₋₃C₃₋₇ carbocyclyl, —(CH₂)₀₋₃ heterocyclyl, —C(O)—(CH₂)₀₋₃ heterocyclyl, —O—(CH₂)₀₋₃ heterocyclyl, —C₂₋₆ alkynyl-heterocyclyl, —C₂₋₆ alkynyl-heterocyclyl-heteraryl, C₆ aryl, and heteroaryl, wherein the alkynyl, alkoxyl, heterocyclyl, heteroalkyl, carbocyclyl, aryl, and heteroaryl is substituted with 0-2 occurrences of halogen, hydroxyl, —(CH₂)₀₋₃C(O)H, —C(O)O-benzyl, —(CH₂)₂₋₆NHR^{c}, heterocyclyl, —O-heterocyclyl, —O-carbocyclyl, —C(O)-heterocyclyl, —C(O)-carbocyclyl, C₁₋₆ alkyl, C₁₋₆ alkoxyl, C₁₋₆ hydroxyalkyl, C₁₋₆ heteroalkyl, and C₁₋₆ haloalkyl;
R^{c} is H, C₁₋₄ alkyl, C₁₋₆ heteroalkyl, and —C(O)OC₁₋₆ alkyl;
R^{d} is H or C₁₋₄ alkyl; or R^{c} and R^{d} together with the nitrogen atom to which they are attached form a heterocyclyl substituted with 0-2 occurrences of —O-heterocyclyl,
R^{p} is H or C₁₋₆ alkyl;
m is 1 or 2; and
n is 1 or 2.

209. The compound of any one of the preceding claims, or a pharmaceutically acceptable salt, hydrate, solvate, prodrug, stereoisomer, or tautomer thereof,

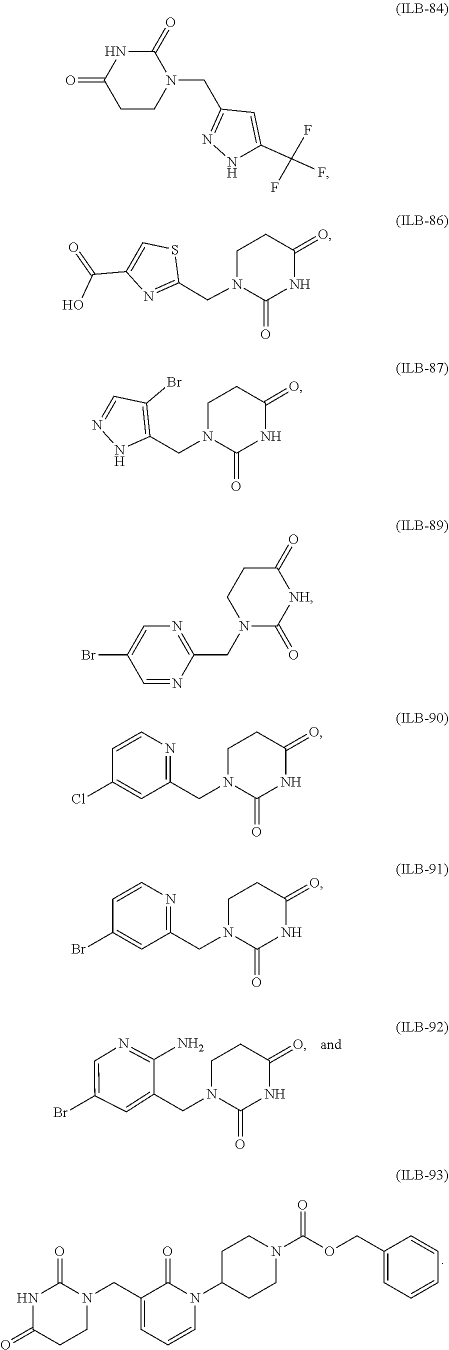

210. A bifunctional compound of Formula (II): 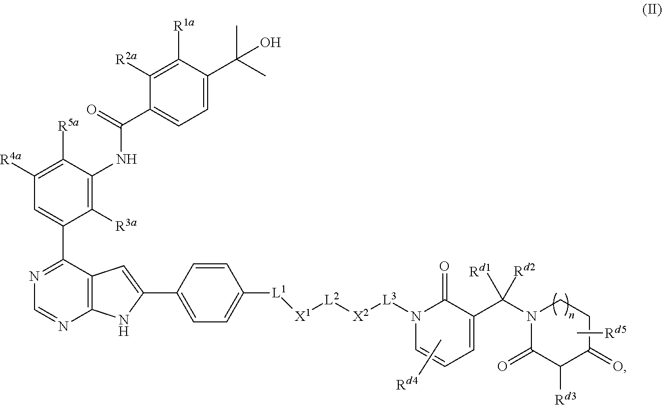
or a pharmaceutically acceptable salt, hydrate, solvate, prodrug, stereoisomer, or tautomer thereof, wherein:
R^{1a} is H or halo;
R^{2a} is halo;
R^{3a} is C₁₋₆ alkyl;
R^{4a} is halo;
R^{5a} is H or halo;
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (BF-III);
wherein no more than 2 of L¹, X¹, X², L², and L³ can simultaneously be a bond;
R′ is hydrogen or C₁₋₆ alkyl;
R^{d1} and R^{d2} are each independently selected from the group consisting of H, C₁₋₆ alkyl, C₁₋₆ haloalkyl, and C₁₋₆ heteroalkyl;
R^{d3} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d4} is selected from the group consisting of H, C₁₋₆ alkyl, halogen, C₁₋₆ haloalkyl, C₁₋₆ alkoxyl, C₁₋₆ alkoxyalkyl, and C₁₋₆ heteroalkyl;
R^{d5} is selected from the group consisting of H, C₁₋₆ alkyl, halogen, C₁₋₆ haloalkyl, and C₁₋₆ heteroalkyl; and
R^{p} is H or C₁₋₆ alkyl.

211. A bifunctional compound of Formula (IIA): 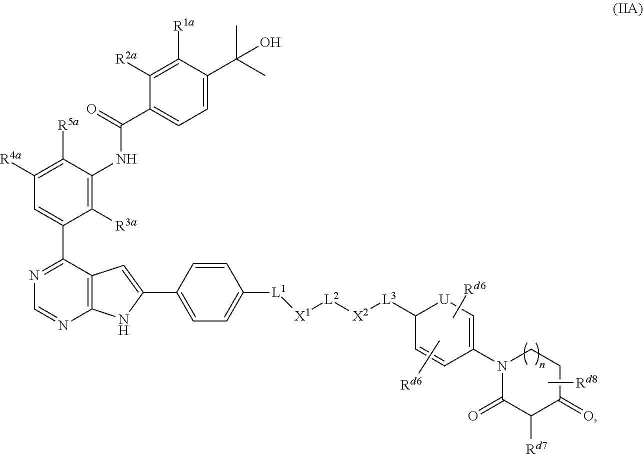
or a pharmaceutically acceptable salt, hydrate, solvate, prodrug, stereoisomer, or tautomer thereof, wherein:
R^{1a} is H or halo;
R^{2a} is halo;
R^{3a} is C₁₋₆ alkyl;
R^{4a} is halo;
R^{5a} is H or halo;
L¹ is selected from the group consisting of a bond, —O—, —NR′—, —C(O)—, C₁₋₉ alkylene, C₁₋₉ heteroalkylene, *C(O)—C₁₋₆ alkylene, *C(O)—C₁₋₆ heteroalkylene, *C₁₋₆ alkylene-C(O), and *C₁₋₆ heteroalkylene-C(O), wherein * denotes the point of attachment of L¹ to the Targeting Ligand;
X¹ and X² are each independently selected from the group consisting of a bond, carbocyclyl, heterocyclyl, and heteroaryl;
L² is selected from the group consisting of a bond, —O—, —NR′—, —C(O)—, C₁₋₆ alkylene, C₁₋₆ heteroalkylene, and *C(O)NR′—C₁₋₆ alkylene, wherein * denotes the point of attachment of L² to X²; or
X¹-L²-X² form a spiroheterocyclyl;
L³ is selected from the group consisting of a bond, C₁₋₆ alkylene, C₂₋₆ alkenylene, C₂₋₆ alkynylene, C₁₋₆ heteroalkylene, —C(O)—, —S(O)₂—, —O—, *C(O)—C₁₋₉ alkylene, and *C(O)—C₁₋₉ heteroalkylene, wherein * denotes the point of attachment of L³ to X² in Formula (BF-III);
wherein no more than 2 of L¹, X¹, X², L², and L³ can simultaneously be a bond;
R′ is hydrogen or C₁₋₆ alkyl;
U is —CR^{d6} or N;
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
U is —CR^{d6} or N;
each R^{d6} is independently selected from the group consisting of H, oxo, C₁₋₆ alkyl, halogen, C₁₋₆ alkoxyl, C₁₋₆ haloalkyl, and C₁₋₆ heteroalkyl;
R^{d7} is selected from the group consisting of H, —CH₂OC(O)R^{p}, —CH₂OP(O)OHOR^{p}, —CH₂OP(O)(R^{p})₂, and —CH₂OP(O)(OR^{p})₂;
R^{d8} is selected from the group consisting of H, C₁₋₆ alkyl, halogen, C₁₋₆ haloalkyl, and C₁₋₆ heteroalkyl;
R^{p} is H or C₁₋₆ alkyl; and
n is 1 or 2.

212. The bifunctional compound of any one of the preceding claims, or a pharmaceutically acceptable salt, hydrate, solvate, prodrug, stereoisomer, or tautomer thereof, wherein R^{2a} is fluoro.

213. The bifunctional compound of any one of the preceding claims, or a pharmaceutically acceptable salt, hydrate, solvate, prodrug, stereoisomer, or tautomer thereof, wherein R^{3a} is C₁₋₃ alkyl.

214. The bifunctional compound of any one of the preceding claims, or a pharmaceutically acceptable salt, hydrate, solvate, prodrug, stereoisomer, or tautomer thereof, wherein R^{3a} is methyl.

215. The bifunctional compound of any one of the preceding claims, or a pharmaceutically acceptable salt, hydrate, solvate, prodrug, stereoisomer, or tautomer thereof, wherein R^{4a} is fluoro.

216. The bifunctional compound of any one of the preceding claims, or a pharmaceutically acceptable salt, hydrate, solvate, prodrug, stereoisomer, or tautomer thereof, wherein L¹ is C₁₋₉ alkylene.

217. The bifunctional compound of any one of the preceding claims, or a pharmaceutically acceptable salt, hydrate, solvate, prodrug, stereoisomer, or tautomer thereof, wherein —X¹-L²-X²— is:

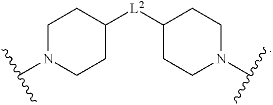

218. The bifunctional compound of any one of the preceding claims, or a pharmaceutically acceptable salt, hydrate, solvate, prodrug, stereoisomer, or tautomer thereof, wherein L² is —C(O)—, —O—, or C₁₋₆ alkylene.

219. The bifunctional compound of any one of the preceding claims, or a pharmaceutically acceptable salt, hydrate, solvate, prodrug, stereoisomer, or tautomer thereof, wherein L³ is selected from the group consisting of a bond, —O—, —C(O)—, —S(O)₂—, C₁₋₆ alkylene, C₂₋₆ alkynylene, and C₁₋₆ heteroalkylene.

220. The bifunctional compound of any one of the preceding claims, or a pharmaceutically acceptable salt, hydrate, solvate, prodrug, stereoisomer, or tautomer thereof, wherein R^{d4} is H.

221. The bifunctional compound of any one of the preceding claims, or a pharmaceutically acceptable salt, hydrate, solvate, prodrug, stereoisomer, or tautomer thereof, wherein R^{d1} is H.

222. The bifunctional compound of any one of the preceding claims, or a pharmaceutically acceptable salt, hydrate, solvate, prodrug, stereoisomer, or tautomer thereof, wherein R^{d2} is H.

223. The bifunctional compound of any one of the preceding claims, or a pharmaceutically acceptable salt, hydrate, solvate, prodrug, stereoisomer, or tautomer thereof, wherein R^{d1} and R^{d2} are both H.

224. The bifunctional compound of any one of the preceding claims, or a pharmaceutically acceptable salt, hydrate, solvate, prodrug, stereoisomer, or tautomer thereof, wherein n is 1.

225. The bifunctional compound of any one of the preceding claims, or a pharmaceutically acceptable salt, hydrate, solvate, prodrug, stereoisomer, or tautomer thereof, wherein R^{d3} is H.

226. The bifunctional compound of any one of the preceding claims, or a pharmaceutically acceptable salt, hydrate, solvate, prodrug, stereoisomer, or tautomer thereof, wherein R^{d5} is H or C₁₋₃ alkyl.

227. The bifunctional compound of any one of the preceding claims, or a pharmaceutically acceptable salt, hydrate, solvate, prodrug, stereoisomer, or tautomer thereof, wherein R^{d5} is H.

228. A bifunctional compound, pharmaceutically acceptable salt, hydrate, solvate, prodrug, stereoisomer, or tautomer thereof, selected from the group consisting of:

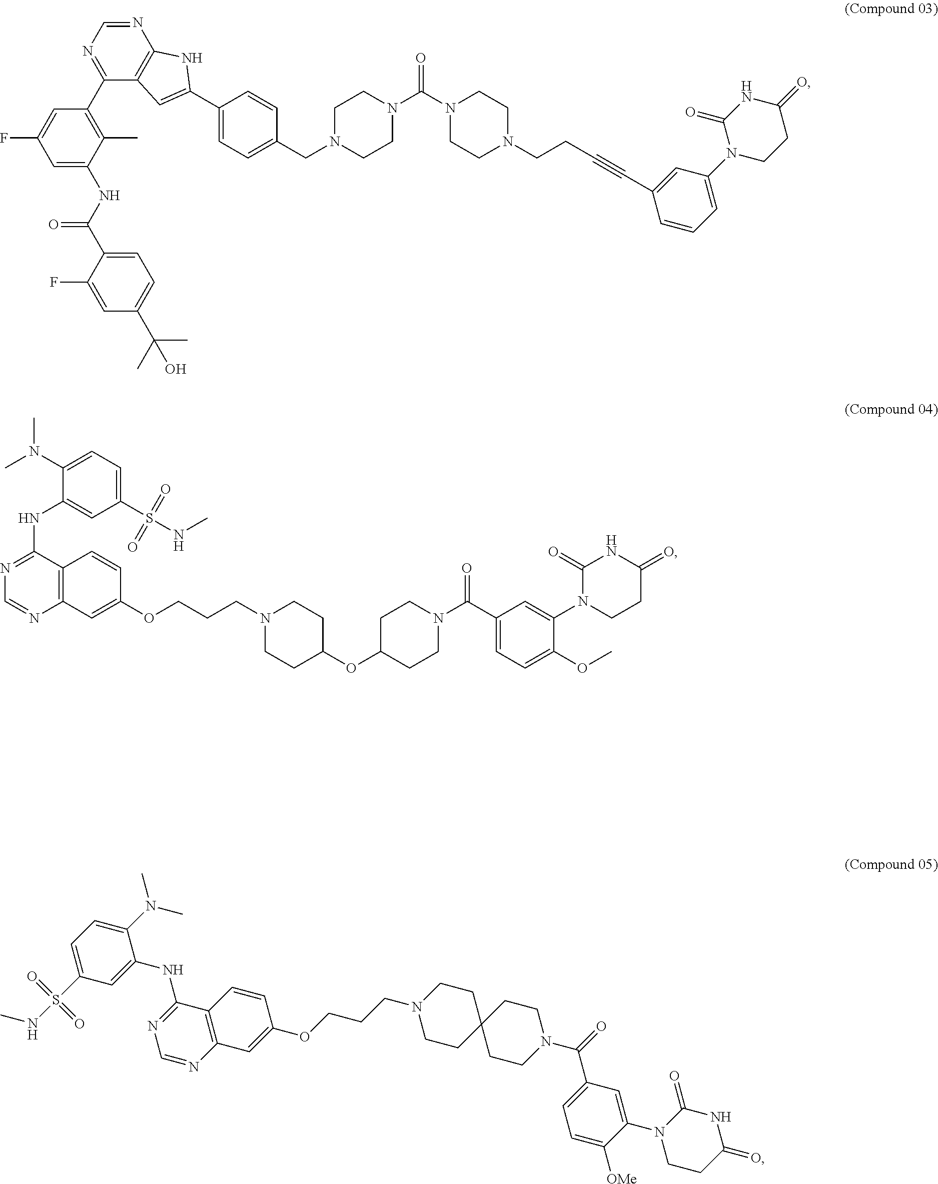 [SMILES: Cc1c(NC(=O)c2ccc(C(C)(C)O)cc2F)cc(F)cc1-c1ncnc2[nH]c(-c3ccc(CN4CCN(C(=O)N5CCN(CCC#Cc6cccc(N7CCC(=O)NC7=O)c6)CC5)CC4)cc3)cc12 ; CNS(=O)(=O)c1ccc(N(C)C)c(Nc2ncnc3cc(OCCCN4CCC(OC5CCN(C(=O)c6ccc(OC)c(N7CCC(=O)NC7=O)c6)CC5)CC4)ccc23)c1 ; CNS(=O)(=O)c1ccc(N(C)C)c(Nc2ncnc3cc(OCCCN4CCC5(CC4)CCN(C(=O)c4ccc(OC)c(N6CCC(=O)NC6=O)c4)CC5)ccc23)c1]

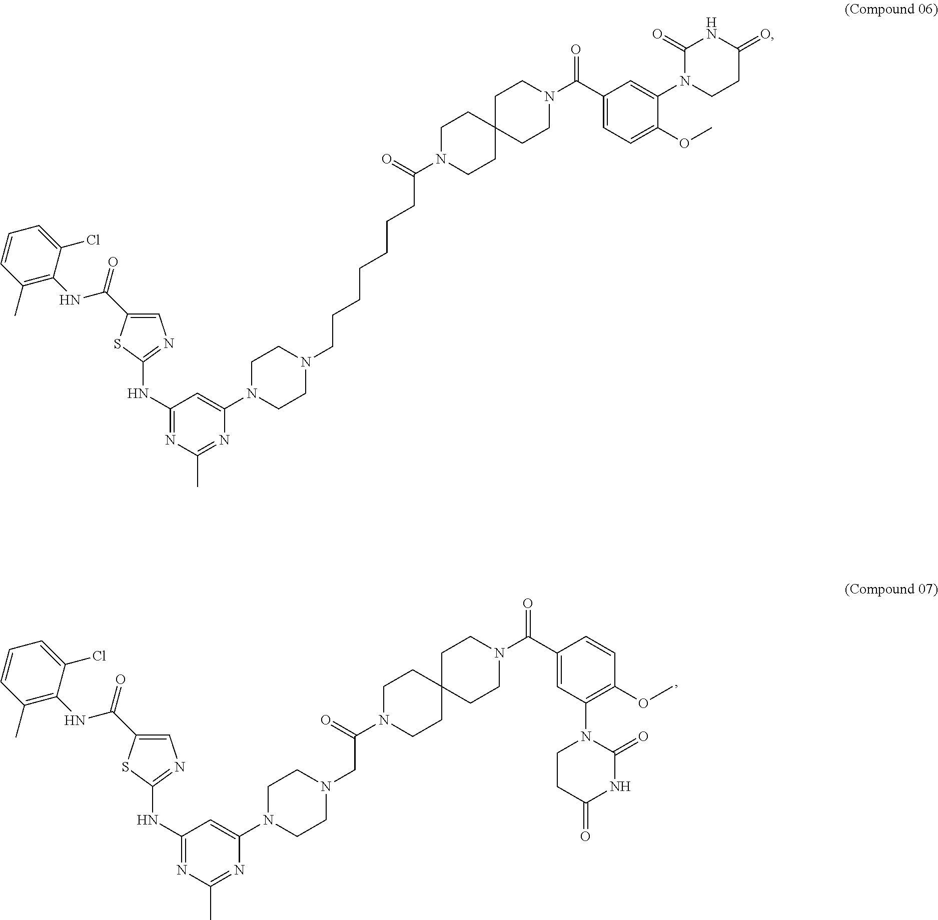 [SMILES: COc1ccc(C(=O)N2CCC3(CCN(C(=O)CCCCCCCN4CCN(c5cc(Nc6ncc(C(=O)Nc7c(C)cccc7Cl)s6)nc(C)n5)CC4)CC3)CC2)cc1N1CCC(=O)NC1=O ; COc1ccc(C(=O)N2CCC3(CCN(C(=O)CN4CCN(c5cc(Nc6ncc(C(=O)Nc7c(C)cccc7Cl)s6)nc(C)n5)CC4)CC3)CC2)cc1N1CCC(=O)NC1=O]

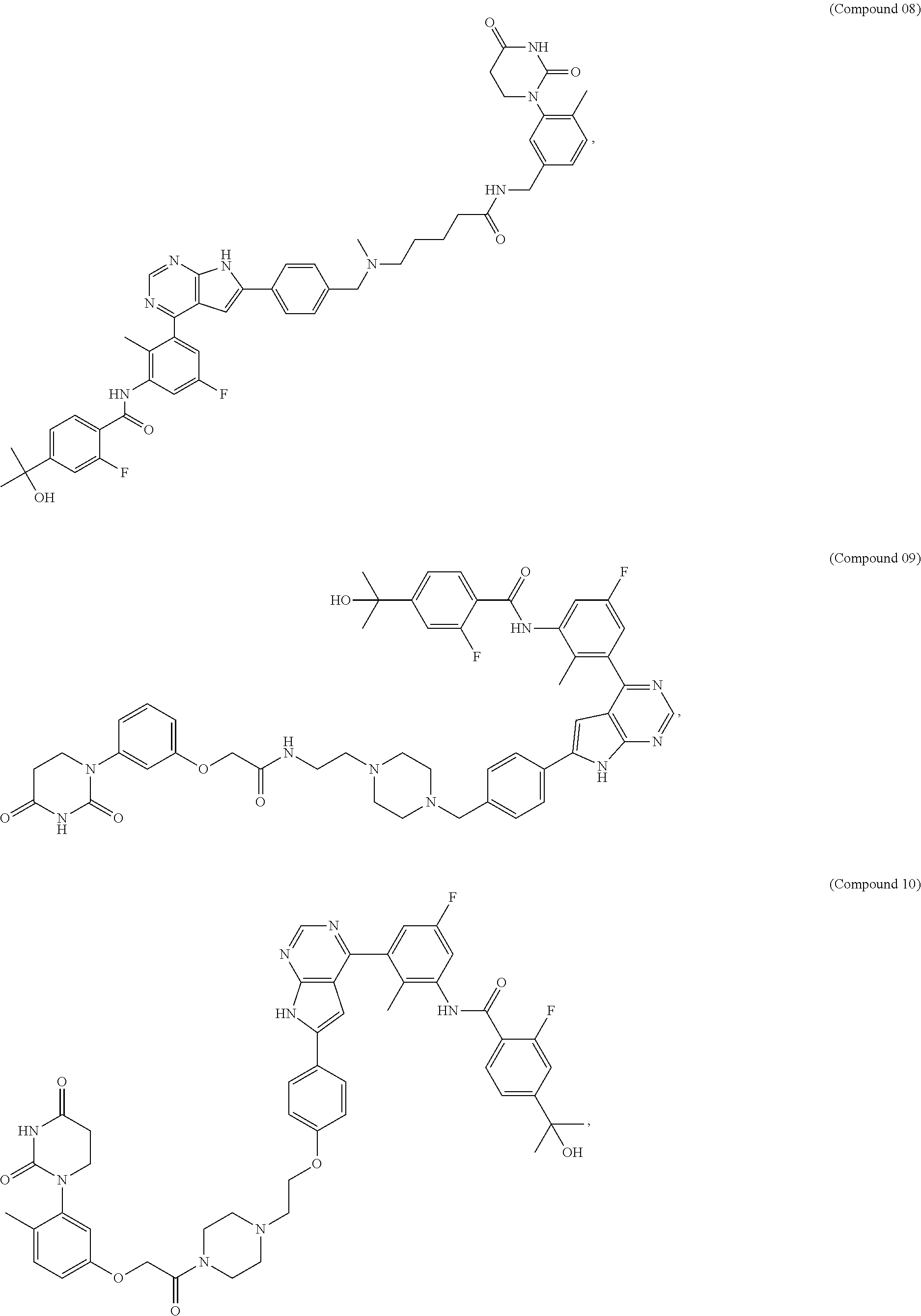

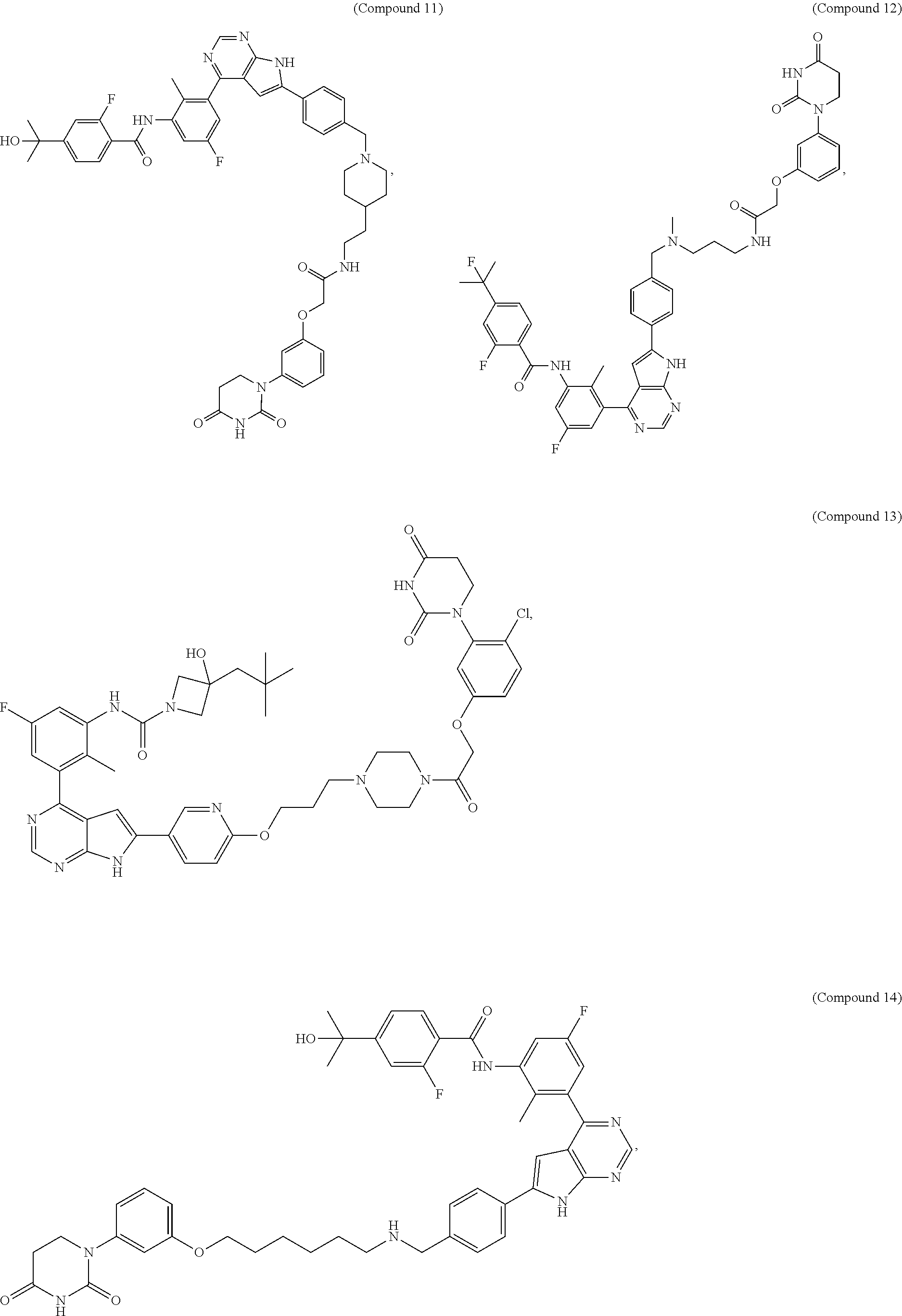 [SMILES: Cc1c(NC(=O)c2ccc(C(C)(C)O)cc2F)cc(F)cc1-c1ncnc2[nH]c(-c3ccc(CN4CCC(CCNC(=O)COc5cccc(N6CCC(=O)NC6=O)c5)CC4)cc3)cc12 ; Cc1c(NC(=O)c2ccc(C(C)(C)F)cc2F)cc(F)cc1-c1ncnc2[nH]c(-c3ccc(CN(C)CCCNC(=O)COc4cccc(N5CCC(=O)NC5=O)c4)cc3)cc12 ; Cc1c(NC(=O)N2CC(O)(CC(C)(C)C)C2)cc(F)cc1-c1ncnc2[nH]c(-c3ccc(OCCCN4CCN(C(=O)COc5ccc(Cl)c(N6CCC(=O)NC6=O)c5)CC4)nc3)cc12 ; Cc1c(NC(=O)c2ccc(C(C)(C)O)cc2F)cc(F)cc1-c1ncnc2[nH]c(-c3ccc(CNCCCCCCOc4cccc(N5CCC(=O)NC5=O)c4)cc3)cc12]

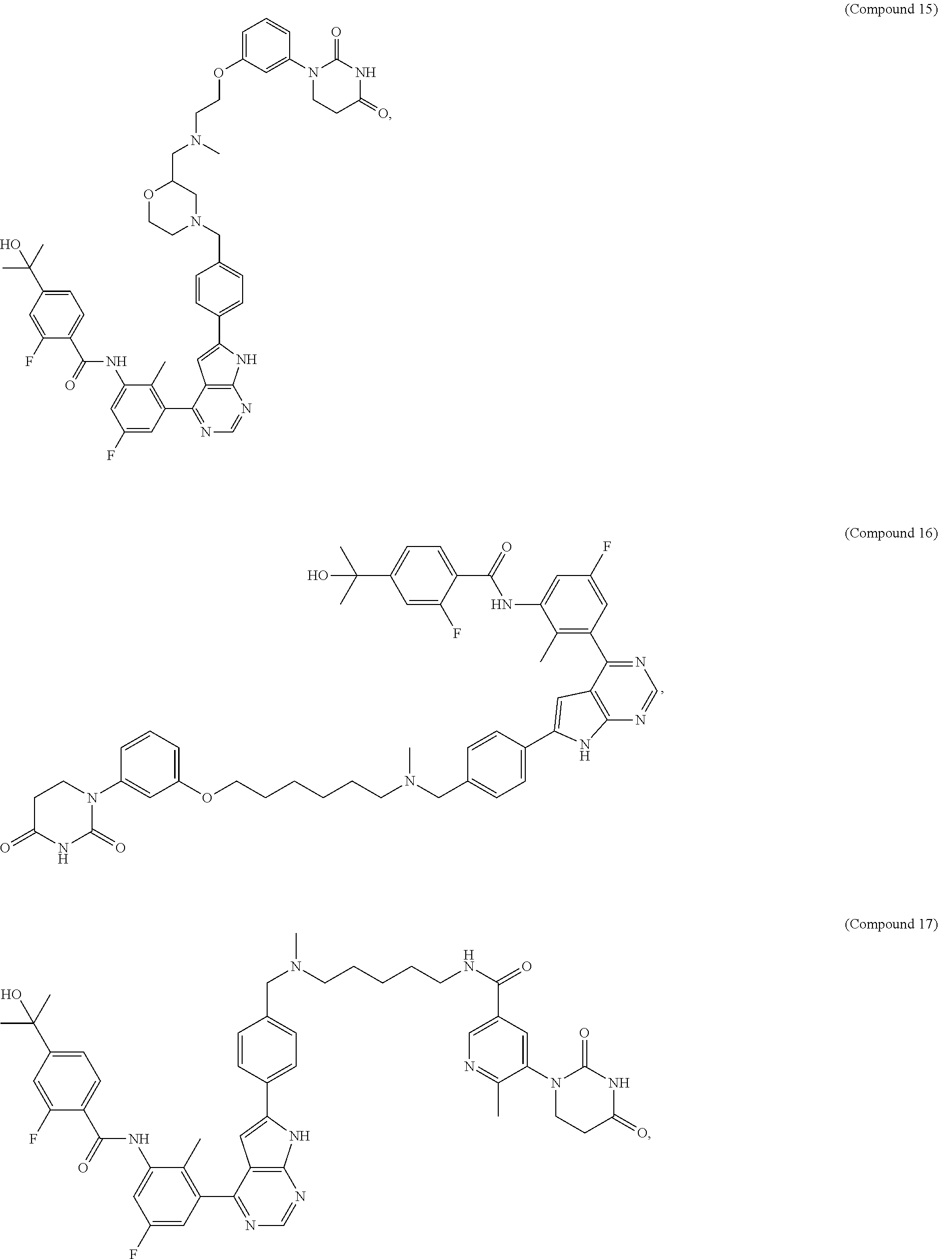 [SMILES: Cc1c(NC(=O)c2ccc(C(C)(C)O)cc2F)cc(F)cc1-c1ncnc2[nH]c(-c3ccc(CN4CCOC(CN(C)CCOc5cccc(N6CCC(=O)NC6=O)c5)C4)cc3)cc12 ; Cc1c(NC(=O)c2ccc(C(C)(C)O)cc2F)cc(F)cc1-c1ncnc2[nH]c(-c3ccc(CN(C)CCCCCCOc4cccc(N5CCC(=O)NC5=O)c4)cc3)cc12 ; Cc1ncc(C(=O)NCCCCCN(C)Cc2ccc(-c3cc4c(-c5cc(F)cc(NC(=O)c6ccc(C(C)(C)O)cc6F)c5C)ncnc4[nH]3)cc2)cc1N1CCC(=O)NC1=O]

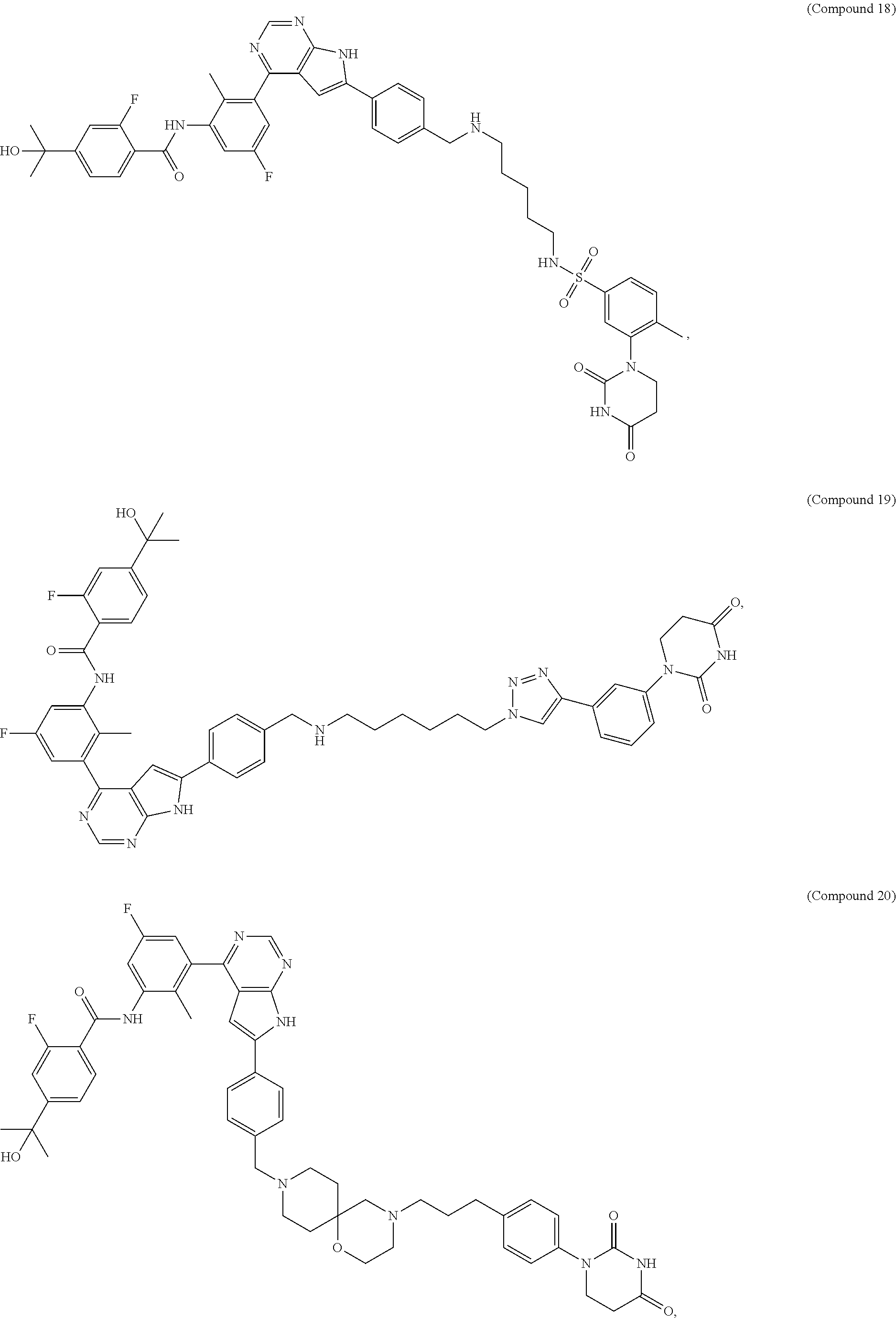

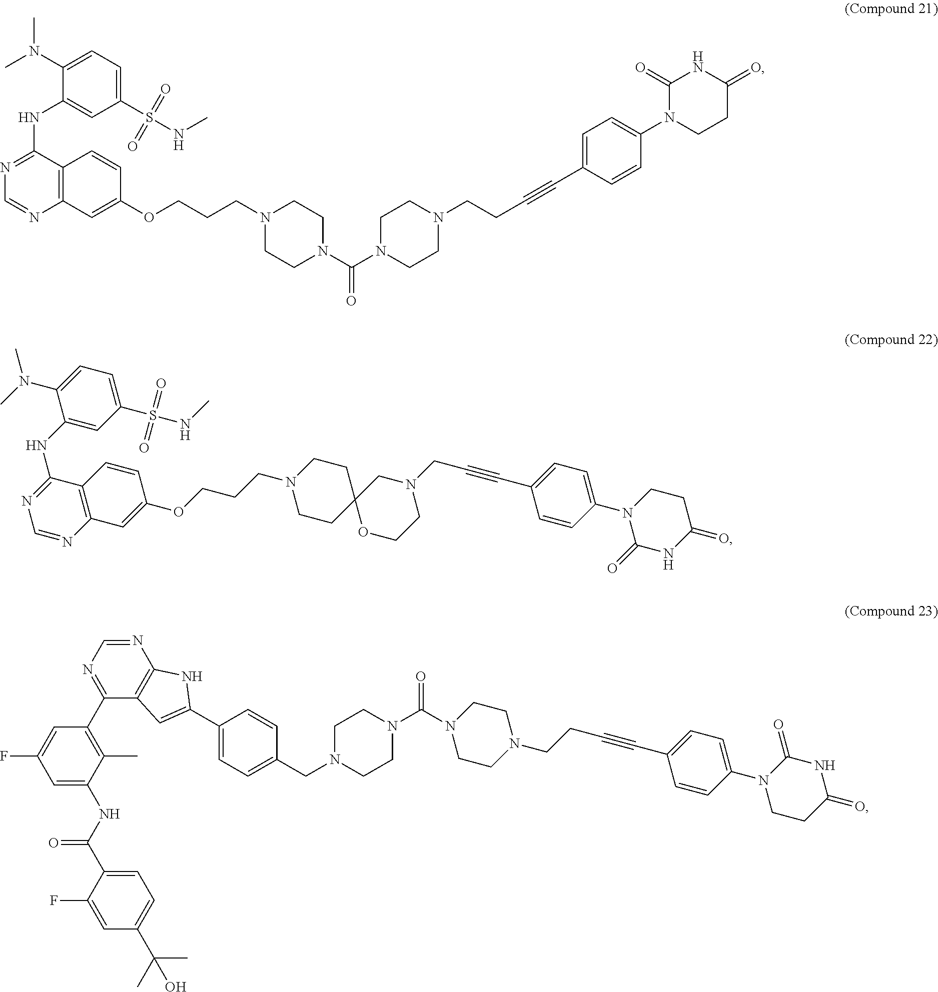 [SMILES: CNS(=O)(=O)c1ccc(N(C)C)c(Nc2ncnc3cc(OCCCN4CCN(C(=O)N5CCN(CCC#Cc6ccc(N7CCC(=O)NC7=O)cc6)CC5)CC4)ccc23)c1 ; CNS(=O)(=O)c1ccc(N(C)C)c(Nc2ncnc3cc(OCCCN4CCC5(CC4)CN(CC#Cc4ccc(N6CCC(=O)NC6=O)cc4)CCO5)ccc23)c1 ; Cc1c(NC(=O)c2ccc(C(C)(C)O)cc2F)cc(F)cc1-c1ncnc2[nH]c(-c3ccc(CN4CCN(C(=O)N5CCN(CCC#Cc6ccc(N7CCC(=O)NC7=O)cc6)CC5)CC4)cc3)cc12]

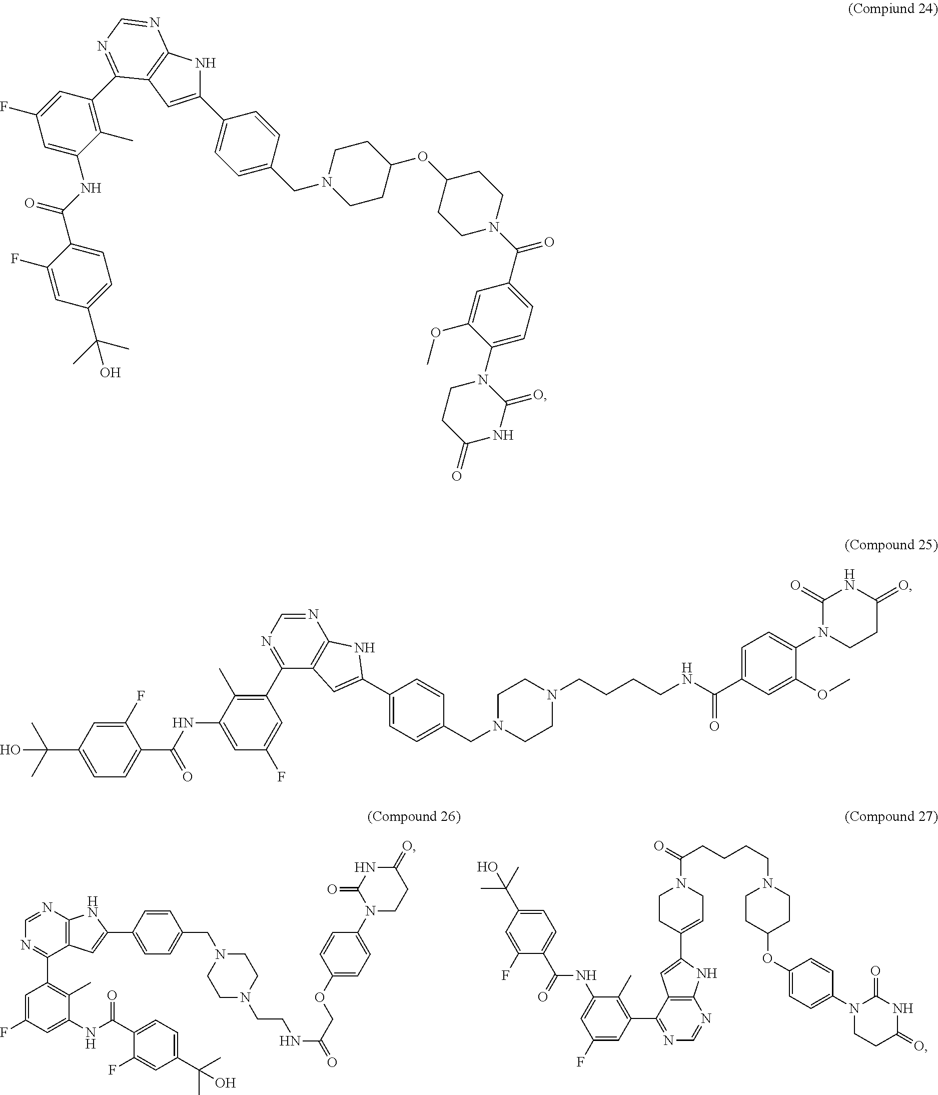 [SMILES: COc1cc(C(=O)N2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O ; COc1cc(C(=O)NCCCCN2CCN(Cc3ccc(-c4cc5c(-c6cc(F)cc(NC(=O)c7ccc(C(C)(C)O)cc7F)c6C)ncnc5[nH]4)cc3)CC2)ccc1N1CCC(=O)NC1=O ; Cc1c(NC(=O)c2ccc(C(C)(C)O)cc2F)cc(F)cc1-c1ncnc2[nH]c(-c3ccc(CN4CCN(CCNC(=O)COc5ccc(N6CCC(=O)NC6=O)cc5)CC4)cc3)cc12 ; Cc1c(NC(=O)c2ccc(C(C)(C)O)cc2F)cc(F)cc1-c1ncnc2[nH]c(C3=CCN(C(=O)CCCCN4CCC(Oc5ccc(N6CCC(=O)NC6=O)cc5)CC4)CC3)cc12]

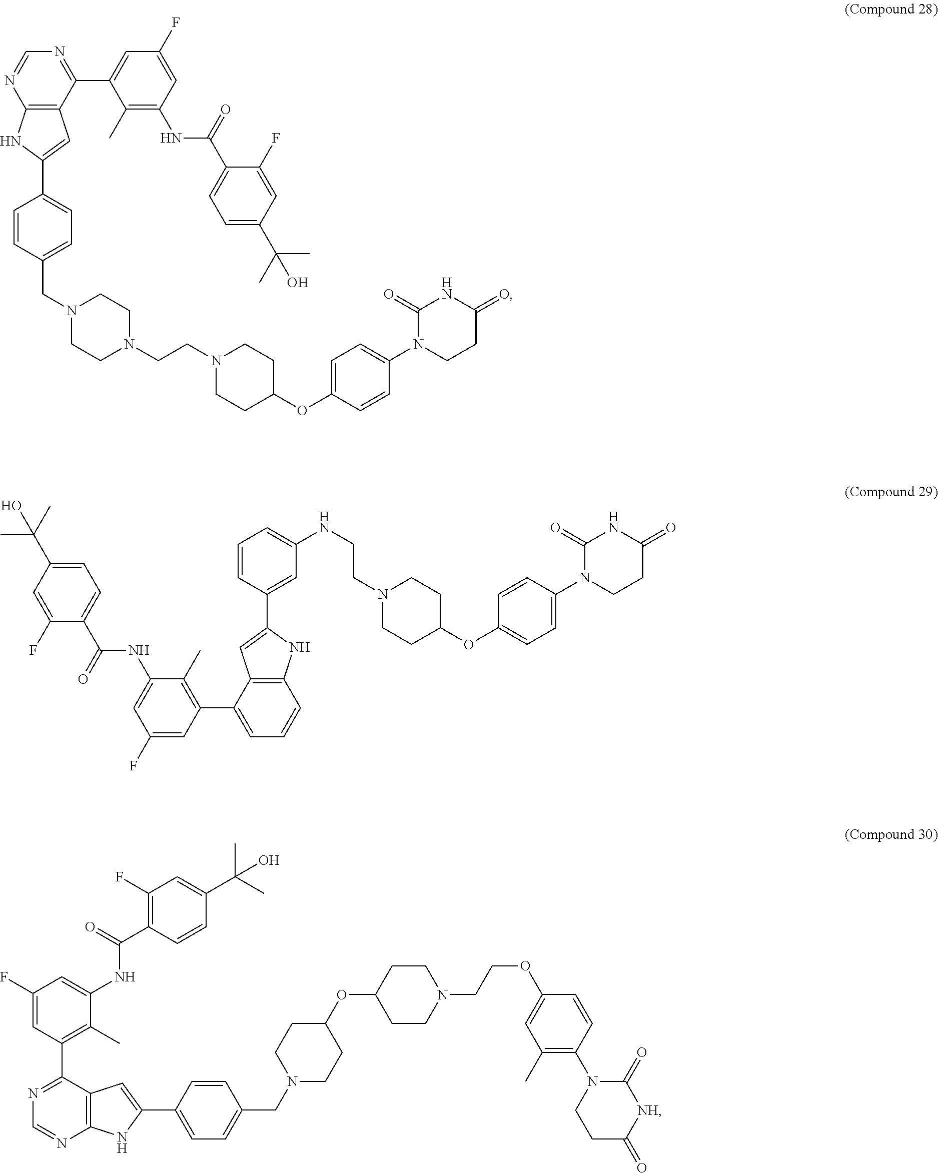 [SMILES: Cc1c(NC(=O)c2ccc(C(C)(C)O)cc2F)cc(F)cc1-c1ncnc2[nH]c(-c3ccc(CN4CCN(CCN5CCC(Oc6ccc(N7CCC(=O)NC7=O)cc6)CC5)CC4)cc3)cc12 ; Cc1c(NC(=O)c2ccc(C(C)(C)O)cc2F)cc(F)cc1-c1cccc2[nH]c(-c3cccc(NCCN4CCC(Oc5ccc(N6CCC(=O)NC6=O)cc5)CC4)c3)cc12 ; Cc1cc(OCCN2CCC(OC3CCN(Cc4ccc(-c5cc6c(-c7cc(F)cc(NC(=O)c8ccc(C(C)(C)O)cc8F)c7C)ncnc6[nH]5)cc4)CC3)CC2)ccc1N1CCC(=O)NC1=O]

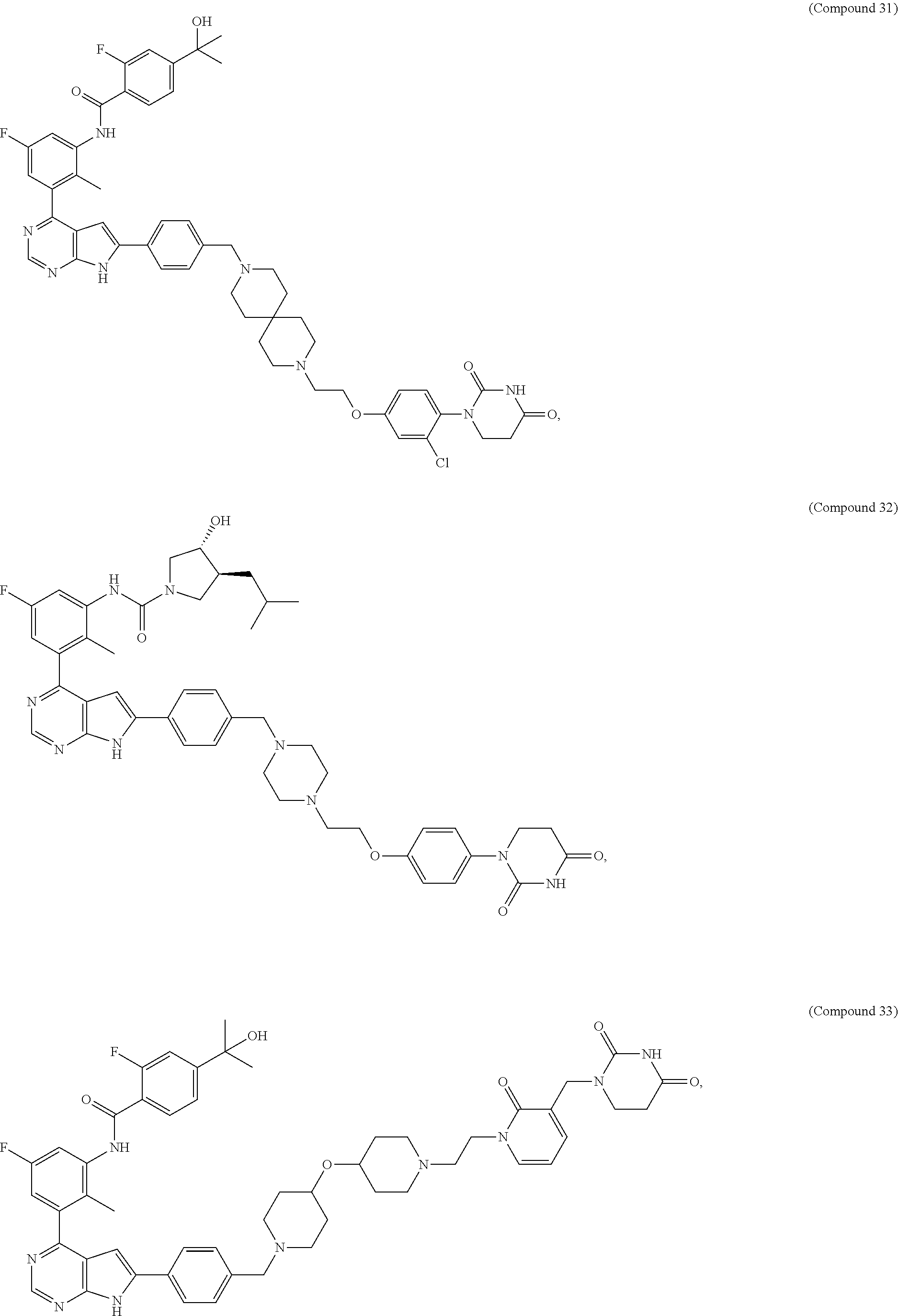 [SMILES: Cc1c(NC(=O)c2ccc(C(C)(C)O)cc2F)cc(F)cc1-c1ncnc2[nH]c(-c3ccc(CN4CCC5(CCN(CCOc6ccc(N7CCC(=O)NC7=O)c(Cl)c6)CC5)CC4)cc3)cc12 ; Cc1c(NC(=O)N2C[C@H](CC(C)C)[C@@H](O)C2)cc(F)cc1-c1ncnc2[nH]c(-c3ccc(CN4CCN(CCOc5ccc(N6CCC(=O)NC6=O)cc5)CC4)cc3)cc12 ; Cc1c(NC(=O)c2ccc(C(C)(C)O)cc2F)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

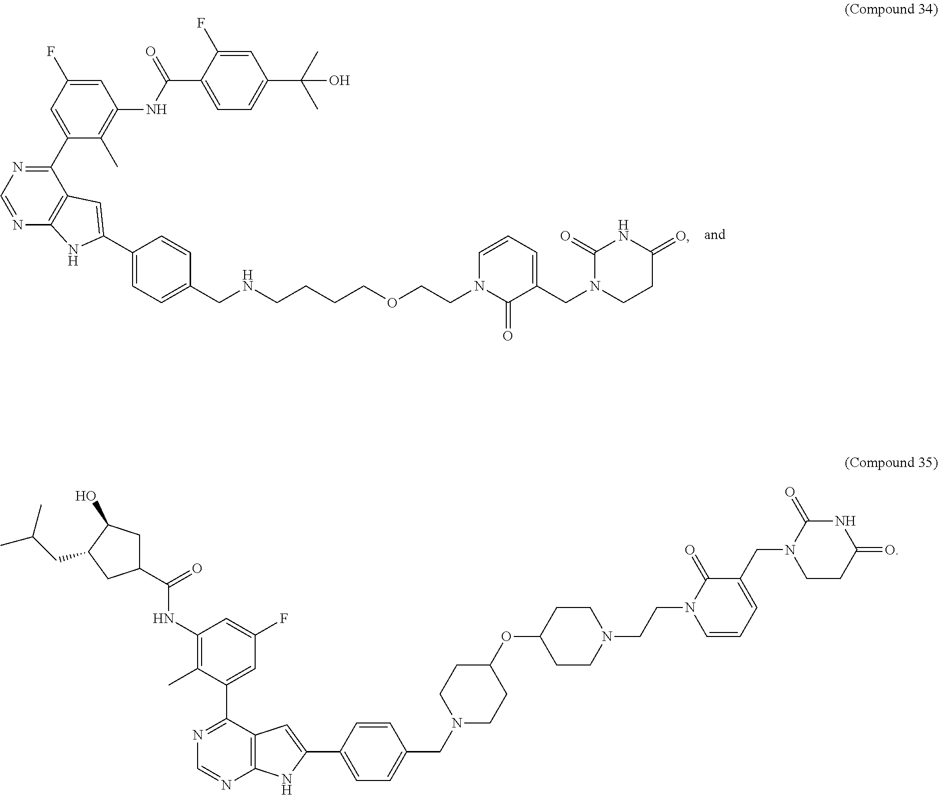 [SMILES: Cc1c(NC(=O)c2ccc(C(C)(C)O)cc2F)cc(F)cc1-c1ncnc2[nH]c(-c3ccc(CNCCCCOCCn4cccc(CN5CCC(=O)NC5=O)c4=O)cc3)cc12 ; Cc1c(NC(=O)C2C[C@H](CC(C)C)[C@@H](O)C2)cc(F)cc1-c1ncnc2[nH]c(-c3ccc(CN4CCC(OC5CCN(CCn6cccc(CN7CCC(=O)NC7=O)c6=O)CC5)CC4)cc3)cc12]

229. A pharmaceutical composition comprising a compound of any one of the preceding claims, or a pharmaceutically acceptable salt, hydrate, solvate, prodrug, stereoisomer, or tautomer thereof, and a pharmaceutically acceptable carrier.

230. A pharmaceutical combination comprising a compound of any one of the preceding claims, or a pharmaceutically acceptable salt, hydrate, solvate, prodrug, stereoisomer, or tautomer thereof, and a therapeutic agent.

231. A method of treating or preventing a respiratory disorder, a proliferative disorder, an autoimmune disorder, an autoinflammatory disorder, an inflammatory disorder, a neurological disorder, and an infectious disease or disorder in a subject in need thereof, the method comprising administering to the subject a therapeutically effective amount of a compound of any one of the preceding claims, or a pharmaceutically acceptable salt thereof.

232. The method of any one of the preceding claims, wherein the disorder is a proliferative disorder.

233. The method of any one of the preceding claims, wherein the proliferative disorder is cancer.

234. Use of a compound of any one of the preceding claims, or a pharmaceutically acceptable salt, hydrate, solvate, prodrug, stereoisomer, or tautomer thereof in the preparation of a medicament for treating a respiratory disorder, a proliferative disorder, an autoimmune disorder, an autoinflammatory disorder, an inflammatory disorder, a neurological disorder, and an infectious disease or disorder in a subject in need thereof.

235. Use of a compound of any one of the preceding claims, or a pharmaceutically acceptable salt, hydrate, solvate, prodrug, stereoisomer, or tautomer thereof for treating cancer.
